(19) United States
(12) Patent Application Publication
(10) Pub. No.: US 2024/0167056 A1
(43) Pub. Date: May 23, 2024

(54) CELL SPECIFIC GENE THERAPY DELIVERY COMPOSITIONS AND METHODS FOR TREATING HEARING LOSS

(71) Applicant: Akouos, Inc., Boston, MA (US)

(72) Inventors: Emmanuel John SIMONS, Boston, MA (US); Robert NG, Boston, MA (US); Danielle R. LENZ, Boston, MA (US); Hao CHIANG, Boston, MA (US)

(21) Appl. No.: 18/314,671

(22) Filed: May 9, 2023

Related U.S. Application Data
(60) Provisional application No. 63/339,937, filed on May 9, 2022.

(51) Int. Cl. C12N 15/86; A61K 48/00; C07K 14/47

(52) U.S. Cl. CPC C12N 15/86; A61K 48/0058; A61K 48/0075; C07K 14/47; C12N 2750/14143; C12N 2830/008; C12N 2830/50

(57) ABSTRACT

The present disclosure provides constructs comprising a coding sequence operably linked to a promoter, wherein the coding sequence encodes a polypeptide (e.g., a therapeutic polypeptide). Exemplary constructs include AAV constructs. Also provided are methods of using disclosed constructs for the treatment of hearing loss and/or deafness.

### CROSS-REFERENCE TO RELATED APPLICATIONS

This application claims priority to U.S. Provisional Patent Application Ser. No. 63/339,937, filed May 9, 2022; the entire contents of which are herein incorporated by reference.

### REFERENCE TO SEQUENCE LISTING SUBMITTED ELECTRONICALLY

The content of the electronically submitted sequence listing in XML format (Name: 4833_0160001_SequenceListing_ST26; Size: 268,520 bytes; and Date of Creation: May 9, 2023) filed with the application is incorporated herein by reference in its entirety.

### BACKGROUND

Hearing loss can be conductive (arising from the ear canal or middle ear), sensorineural (arising from the inner ear or auditory nerve), or mixed. Most forms of nonsyndromic deafness are associated with permanent hearing loss caused by damage to structures in the inner ear (sensorineural deafness), although some forms may involve changes in the middle ear (conductive hearing loss). The great majority of human sensorineural hearing loss is caused by abnormalities in the hair cells of the organ of Corti in the cochlea (poor hair cell function). The hair cells may be abnormal at birth, or may be damaged during the lifetime of an individual (e.g., as a result of noise trauma or infection).

### SUMMARY

Certain aspects of the disclosure are directed to polynucleotide comprising a sequence having at least 85%, at least 90%, at least 95%, at least 96%, at least 97%, at least 98%, at least 99%, or 100% identity to any one of SEQ ID NOs: 16, 28, 40, 57, or 90-99. In some aspects, the polynucleotide is a promoter.

Certain aspects of the disclosure are directed to a construct (e.g., an expression construct) comprising the polynucleotide. In some aspects, the construct further comprises a nucleic acid sequence encoding a polypeptide. In some aspects, the polynucleotide is operably linked to the nucleic acid sequence encoding the polypeptide. In some aspects, the polynucleotide promotes expression of the nucleic acid in an inner ear support cell.

Certain aspects of the disclosure are directed to a construct comprising a polynucleotide encoding a therapeutic polypeptide operably linked to a promoter which expresses the polynucleotide in an inner ear support cell. In some aspects, the polynucleotide encodes a therapeutic polypeptide or a reporter polypeptide. In some aspects, the promoter selectively expresses the polynucleotide in an inner ear support cell.

Certain aspects of the disclosure are directed to a construct comprising a polynucleotide encoding a polypeptide operably linked to a promoter which expresses the polynucleotide in an inner ear support cell, wherein the promoter is heterologous to the polynucleotide.

In some aspects, the construct is an expression construct comprising a polynucleotide encoding a polypeptide operably linked to an inner ear supporting cell selective promoter and a minimal promoter that is homologous to the polypeptide, wherein the polynucleotide is expressed in an inner ear support cell.

Certain aspects of the disclosure are directed to a construct comprising a polynucleotide encoding a polypeptide operably linked to a promoter, wherein the promoter comprises a nucleic acid sequence having at least 80%, at least 85%, at least 90%, at least 95%, at least 96%, at least 97%, at least 98%, at least 99%, or 100% identity to any one of SEQ ID NOs: 16, 28, 40, 57, 90-99. In some aspects, the promoter is heterologous to the polynucleotide.

Certain aspects of the disclosure are directed to a construct comprising a polynucleotide encoding a polypeptide, an inner ear supporting cell selective promoter and a minimal GJB2 promoter, wherein the polynucleotide is operably linked to the inner ear supporting cell selective promoter and the minimal GJB2 promoter such that the polynucleotide is expressed in an inner ear support cell, wherein the inner ear supporting cell selective promoter is heterologous to the polynucleotide.

Certain aspects of the disclosure are directed to a construct comprising a polynucleotide encoding a polypeptide, an inner ear supporting cell selective promoter and a minimal GJB2 promoter, whererin the polynucleotide is operably linked to the inner ear supporting cell selective promoter and the minimal GJB2 promoter, wherein the inner ear supporting cell selective promoter comprises a nucleic acid sequence having at least 80%, at least 85%, at least 90%, at least 95%, at least 96%, at least 97%, at least 98%, at least 99%, or 100% identity to any one of SEQ ID NOs: 16, 28, 40, 57, 90-99. In some aspects, the inner ear supporting cell selective promoter is heterologous to the polynucleotide. In some aspects, the minimal GJB2 promoter comprises a nucleic acid sequence at least 80%, at least 85%, at least 90%, at least 95%, at least 96%, at least 97%, at least 98%, at least 99%, or 100% identity to SEQ ID NO: 86.

Certain aspects of the disclosure are directed to a construct comprising: (i) a 5′ inverted terminal repeat (ITR), (ii) a polynucleotide encoding a polypeptide operably linked to a promoter which expresses the polynucleotide in an inner ear support cell, and (iii) a 3′ ITR, wherein the promoter is heterologous to the polynucleotide.

Certain aspects of the disclosure are directed to a construct comprising: (i) a 5′ inverted terminal repeat (ITR), (ii) a polynucleotide encoding a polypeptide operably linked to a promoter, and (iii) a 3′ ITR, wherein the promoter comprises a nucleic acid sequence having at least 85%, at least 90%, at least 95%, at least 96%, at least 97%, at least 98%, at least 99%, or 100% identity to any one of SEQ ID NOs: 16, 28, 40, 57, or 90-99. In some aspects, the construct further comprises a minimal GJB2 promoter. In some aspects, the minimal GJB2 promoter comprises a nucleic acid sequence at least 80%, at least 85%, at least 90%, at least 95%, at least 96%, at least 97%, at least 98%, at least 99%, or 100% identity to SEQ ID NO: 86.

In some aspects, the promoter is selected from one or more a GJB6 promoter, a GDF6 promoter, a IGFBP2 promoter, a RBP7 promoter, a PARM1 promoter, a GFAP promoter, a BACE2 promoter, a DBI2 promoter, a FABP3 promoter, a KLHL14 promoter, a MMP15 promoter, a SPARC promoter, a TSPAN8 promoter, a VIM promoter, derivatives thereof, or fragments thereof.

In some aspects, the promoter is a GJB2 promoter or a minimal GJB2 promoter.

In some aspects, the construct comprises two or more promoters. In some aspects, the first promoter is selected from a GJB6 promoter, a GDF6 promoter, a IGFBP2 promoter, a RBP7 promoter, a PARM1 promoter, a GFAP promoter, a BACE2 promoter, a DBI2 promoter, a FABP3 promoter, a KLHL14 promoter, a MMP15 promoter, a SPARC promoter, a TSPAN8 promoter, a VIM promoter, or any combination thereof. In some aspects, the second promoter is selected from a GJB2 promoter or a minimal GJB2 promoter.

Certain aspects of the disclosure are directed to a construct comprising: (i) a 5′ inverted terminal repeat (ITR), (ii) a polynucleotide encoding a polypeptide operably linked to an inner ear supporting cell selective promoter and a minimal GJB2 promoter, wherein the polynucleotide is expressed in an inner ear support cell, and (iii) a 3′ ITR, wherein the inner ear supporting cell selective promoter is heterologous to the polynucleotide.

Certain aspects of the disclosure are directed to a construct comprising: (i) a 5′ inverted terminal repeat (ITR), (ii) a polynucleotide encoding a polypeptide operably linked to a inner ear supporting cell selective promoter and a minimal GJB2 promoter, and (iii) a 3′ ITR, wherein the inner ear supporting cell selective promoter comprises a nucleic acid sequence having at least 85%, at least 90%, at least 95%, at least 96%, at least 97%, at least 98%, at least 99%, or 100% identity to any one of SEQ ID NOs: 16, 28, 40, 57, or 90-99.

In some aspects, inner ear supporting cells include, but are not limited to, inner phalangeal cells/border cells (IPhC), inner pillar cells (IPC), outer pillar cells (OPC), Deiters' cells rows 1 and 2 (DC1/2), Deiters' cells row 3 (DC3), Hensen's cells (Hec), Claudius cells/outer sulcus cells (CC/OSC), interdental cells (Idc), inner sulcus cells (ISC), Kölliker's organ cells (KO), greater ridge epithelial ridge cells (GER) (including lateral greater epithelial ridge cells (LGER)), and OC90+ cells (OC90), fibroblasts, and other cells of the lateral wall.

In some aspects, the promoter comprises a nucleic acid sequence having at least 80%, at least 85%, at least 90%, at least 95%, at least 96%, at least 97%, at least 98%, at least 99%, or 100% identity to any one of SEQ ID NOs: 16, 28, 40, 57, 90-99.

In some aspects, the construct comprises a miRNA regulatory target site (miRTS) for a microRNA expressed in an inner ear cell. In some aspects, the microRNA is expressed in an inner ear hair cell. In some aspects, the microRNA is one or more of miR-194, miR-140, miR-18a, miR-99a, miR-30b, miR-15a, miR182, miR-183, or any combination thereof.

Certain aspects of the disclosure are directed to a construct comprising a polynucleotide encoding a polypeptide operably linked to a promoter, wherein the construct comprises a miRNA regulatory target site (miRTS) for a microRNA expressed in an inner ear cell.

In some aspects, the polynucleotide encodes a therapeutic polypeptide or a reporter polypeptide.

In some aspects, the microRNA is expressed in one or more of inner ear hair cells, spiral ganglion cells, lateral supporting cells, basilar membrane cells, medial supporting cells, spiral limbus cells, or inner sulcus cells.

In some aspects, the microRNA is expressed in inner ear hair cells.

In some aspects, the microRNA is one or more of miR-194, miR-140, miR-18a, miR-99a, miR-30b, miR-15a, miR182, or miR-183.

In some aspects, the construct comprises a 5′ and a 3′ inverted terminal repeat (ITR). In some aspects, the construct comprises a 5′ untranslated region (UTR). In some aspects, the construct comprises a 3′ untranslated region (UTR).

Certain aspects of the disclosure are directed to vectors, viral particles (e.g., AAV), ex vivo cells, and compositions comprising the constructs disclosed herein.

Certain aspects of the disclosure are directed to an adeno-associated virus (AAV) particle comprising a construct disclosed herein.

Certainaspects are directed to an adeno-assocciated virus (AAV) particle comprising a construct comprising: (i) a 5′ inverted terminal repeat (ITR), (ii) a polynucleotide encoding a polypeptide operably linked to a promoter which expresses the polynucleotide in an inner ear support cell, and (iii) a 3′ ITR, wherein the promoter is heterologous to the polynucleotide. In some aspects, the promoter comprises a nucleic acid sequence at least 85%, at least 90%, at least 95%, at least 96%, at least 97%, at least 98%, at least 99%, or 100% identical to any one of SEQ ID NOs: 16, 28, 40, 57, or 90-99. In some aspects, the construct further comprises a minimal GJB2 promoter. In some aspects, the minimal GJB2 promoter comprises a nucleic acid sequence at least 80%, at least 85%, at least 90%, at least 95%, at least 96%, at least 97%, at least 98%, at least 99%, or 100% identical to SEQ ID NO: 86.

Certainaspects are directed to an adeno-assocciated virus (AAV) particle comprising a construct comprising: (i) a 5′ inverted terminal repeat (ITR), (ii) a 5′ untranslated region (UTR), (iii) a polynucleotide encoding a polypeptide operably linked to a promoter which expresses the polynucleotide in an inner ear support cell, (iv) a 3′ UTR, and (v) a 3′ ITR, wherein the promoter is heterologous to the polynucleotide.

Certain aspects of the disclosure are directed to an adeno-assocciated virus (AAV) particle comprising a construct comprising: (i) a 5′ inverted terminal repeat (ITR), (ii) a 5′ untranslated region (UTR), (iii) a polynucleotide encoding a polypeptide operably linked to an inner ear supporting cell selective promoter and a minimal GJB2 promoter, wherein the polynucleotide is expressed in an inner ear support cell, (iv) a 3′ UTR, and (v) a 3′ ITR, wherein the inner ear supporting cell selective promoter is heterologous to the polynucleotide

Certain aspects of the disclosure are directed to an adeno-associated virus (AAV) particle comprising a construct comprising: (i) a 5′ inverted terminal repeat (ITR), (ii) a 5′ untranslated region (UTR), (iii) a polynucleotide encoding a polypeptide operably linked to a promoter, (iv) a 3′ UTR, and (v) a 3′ ITR, wherein the promoter comprises a nucleic acid sequence having at least 85%, at least 90%, at least 95%, at least 96%, at least 97%, at least 98%, at least 99%, or 100% identity to any one of SEQ ID NOs: 16, 28, 40, 57, or 90-99.

Certainadeno-associated virus (AAV) particle comprising a construct comprising: (i) a 5′ inverted terminal repeat (ITR), (ii) a 5′ untranslated region (UTR), (iii) a polynucleotide encoding a polypeptide operably linked to a promoter, (iv) a miRNA regulatory target site (miRTS) for a microRNA expressed in an inner ear cell, (v) a 3′ UTR, and (vi) a 3′ ITR.

Certain aspects of the disclosure are directed to an adeno-associated virus (AAV) particle comprising a construct comprising: (i) a 5′ inverted terminal repeat (ITR), (ii) a 5′ untranslated region (UTR), (iii) a polynucleotide encoding a polypeptide operably linked to an inner ear supporting cell selective promoter and minimal GJB2 promoter, (iv) a 3′ UTR, and (v) a 3′ ITR, wherein the inner ear supporting cell selective promoter comprises a nucleic acid sequence having at least 85%, at least 90%, at least 95%, at least 96%, at least 97%, at least 98%, at least 99%, or 100% identity to any one of SEQ ID NOs: 16, 28, 40, 57, or 90-99.

Certainadeno-associated virus (AAV) particle comprising a construct comprising: (i) a 5′ inverted terminal repeat (ITR), (ii) a 5′ untranslated region (UTR), (iii) a polynucleotide encoding a polypeptide operably linked to an inner ear supporting cell selective promoter and a minimal GJB2 promoter, (iv) a miRNA regulatory target site (miRTS) for a microRNA expressed in an inner ear cell, (v) a 3′ UTR, and (vi) a 3′ ITR.

In some aspects, the inner ear supporting cell selective promoter is selected from one or more a GJB6 promoter, a GDF6 promoter, a IGFBP2 promoter, a RBP7 promoter, a PARM1 promoter, a GFAP promoter, a BACE2 promoter, a DBI2 promoter, a FABP3 promoter, a KLHL14 promoter, a MMP15 promoter, a SPARC promoter, a TSPAN8 promoter, a VIM promoter, derivatives thereof, or fragments thereof.

In some aspects, the minimal GJB2 promoter comprises a nucleic acid sequence with at least 85%, at least 90%, at least 95%, at least 96%, at least 97%, at least 98% at least 99%, or 100% identity to SEQ ID NO: 86.

Certain aspects of the disclosure are directed to methods of using the constructs, vectors, viral particles (e.g., AAV), ex vivo cells, and compositions disclosed herein for expressing a polypeptide in an inner ear cell (e.g., a supporting cells).

Certain aspects of the disclosure are directed to methods of using the constructs, vectors, viral particles (e.g., AAV), ex vivo cells, and compositions disclosed herein for increasing expression of a polypeptide (e.g., a therapeutic polypeptide) in an inner ear cell (e.g., a supporting cells). In some aspects, the increasing expression is relative to the corresponding endogenous polypeptide expression in the inner ear cell (e.g., a supporting cells).

Certain aspects of the disclosure are directed to methods of using the constructs, vectors, viral particles (e.g., AAV), ex vivo cells, and compositions disclosed herein for decreasing expression of a polypeptide (e.g., a therapeutic polypeptide) in non-inner ear supporting cells (e.g., inner ear hair cells). In some aspects, the decreasing expression is relative to the corresponding endogenous polypeptide expression in the non-inner ear cell supporting cells (e.g., inner ear hair cells).

Certain aspects of the disclosure are directed to methods of using the constructs, vectors, viral particles (e.g., AAV), ex vivo cells, and compositions disclosed herein for reducing toxicity associated with expression of a polypeptide, (e.g., a therapeutic polypeptide) in an inner ear cell.

Certain aspects of the disclosure are directed to methods of using the constructs, vectors, viral particles (e.g., AAV), ex vivo cells, and compositions disclosed herein for treating hearing loss in a subject suffering from or at risk of hearing loss.

Certain aspects of the disclosure are directed to a kit comprising the expression or viral vector construct, the vector, the AAV particle, the composition, or the ex vivo cell disclosed herein.

In some aspects, the construct, vector, AAV particle, composition or ex vivo cell disclosed herein is pre-loaded in a device. In some aspects, the device is a microcatheter.

In some aspects, the microcatheter is shaped such that it can enter the middle ear cavity via the external auditory canal and contact the end of the microcatheter with the RWM. In some aspects, a distal end of the microcatheter is comprised of at least one microneedle with diameter of between 10 and 1,000 microns. In some aspects, the device is a device described in any one of FIGS. 5-8. In some aspects, the device comprises a needle comprising a bent portion and an angled tip.

In some aspects, the polynucleotide, the construct, the expression construct, the viral vector construct, the viral vector, the AAV particle, the ex vivo cell, the method, the use or the kit disclosed herein comprises a polynucleotide or a promoter which is a cell selective promoter. In some aspects, the cell selective promoter is a promoter that is predominately active in one or more supporting cells of the inner ear.

### BRIEF DESCRIPTION OF THE DRAWINGS

FIG. 1A depicts a simplified endogenous AAV genome. FIG. 1B depicts a simplified recombinant AAV (rAAV) construct capable of expressing a therapeutic polypeptide (e.g., a GJB2 gene).

FIGS. 2A-2H depict alternative exemplary rAAV constructs comprising a therapeutic polypeptide FIG. 2A depicts an exemplary rAAV construct comprising a 5′ ITR, a CAG promoter, a nucleic acid encoding a therapeutic polypeptide (a hGJB2 gene), a bGH polyA, and a 3′ ITR. FIG. 2B depicts an exemplary rAAV construct comprising a 5′ ITR, a CAG promoter, a nucleic acid encoding a therapeutic polypeptide (a hGJB2 gene), a 3′ UTR, a bGH polyA, and a 3′ ITR. FIG. 2C depicts an exemplary rAAV construct comprising a 5′ ITR, a CAG promoter, a 5′ UTR, a nucleic acid encoding a therapeutic polypeptide (a hGJB2 gene), a FLAG tag, a 3′ UTR, a bGH polyA, and a 3′ ITR. FIG. 2D depicts an exemplary rAAV construct comprising a 5′ ITR, a smCBA promoter, a 5′ UTR, a nucleic acid encoding a therapeutic polypeptide (a hGJB2 gene), a FLAG tag, a 3′ UTR, a bGH polyA, and a 3′ ITR. FIG. 2E depicts an exemplary rAAV construct comprising a 5′ ITR, a promoter comprising a CMV promoter and a hGJB2 promoter, a 5′ UTR, a nucleic acid encoding a hGJB2 gene, a FLAG tag, a 3′ UTR, a bGH polyA, and a 3′ ITR. FIG. 2F depicts an exemplary rAAV construct comprising a 5′ ITR, a CAG promoter, a 5′ UTR, a hGJB2 promoter, a FLAG tag, a microRNA regulatory target site, a 3′ UTR, a bGH polyA, and a 3′ ITR. FIG. 2G depicts an exemplary rAAV construct comprising a 5′ ITR, a promoter comprising an inner ear supporting cell selective promoter and a hGJB2 minimal promoter, a nucleic acid encoding a therapeutic polypeptide (a hGJB2 gene), a FLAG tag, a 5′ UTR, a bGH polyA, and a 3′ ITR. FIG. 2H depicts an exemplary rAAV construct comprising a 5′ ITR, a CAG promoter, a nucleic acid encoding a therapeutic polypeptide (a hGJB2 gene), a FLAG tag, a T2A element, a nucleic acid encoding eGFP, a bGH polyA, and a 3′ ITR.

FIGS. 3A-Q depict in vitro or ex vivo expression of a transgene in HEK293FT cells transfected or transduced with a contruct with a microRNA targeting site (miRTS) in the presence or absence of a microRNA recognizing that site. FIG. 3A is a schematic that represents construct comprising a gene of interest and a miRTS. FIG. 3B is a Venn diagram representing selection of miRTS based on expression of microRNAs expressed in the different inner ear cell types. FIG. 3C is a graph showing the GFP expression in cells transfected with miRNA-expressing plasmid (pITR.CAG.mScarlet.miRNA) and a plasmid comprising a gene-of-interest and microRNA target site (pITR.CAG.GOI.miRTS). FIG. 3D is a graph showing GFP expression as measured by flow cytometry in HEK293FT cells transduced with an AAVAnc80 vector comprising GFP and a microRNA target site (AAVAncO-CAG.GOI.miRTS) and transfected with a plasmid expressing miRNA targeting the miRTS (pITR.CAG.mScarlet.miRNA). FIG. 3E is a graph showing a gene of interest expression measured by RT-qPCR in cells transduced with an AAVAnc80 expressing the gene of interest with a microRNA targeting site (AAVAnc80-CAG.GOI.miRTS) following transfection with either of two amounts of plasmid expressing a plasmid encoding a miRNA targeting the miRTS (pITR.CAG.mScarlet.miRNA). FIG. 3F is a western blot of protein showing expression of the gene of interest in cells transduced with an AAVAnc80 comprising the gene of interest and a microRNA targeting site (AAVAnc80-CAG.GOI.miRTS) following transfection with either of two amounts of plasmid expressing a miRNA targeting the miRTS (pITR.CAG.mScarlet.miRNA). FIG. 3G is a graph showing the quantification of proteins levels determined from the western blot in FIG. 3F. FIG. 3H is a heat map of gene expression due to in vitro transduction of the gene-of-interest with the microRNA targeting site compared to transduction with the gene-of-interest alone. FIG. 3I is a volcano plot displaying differential gene expression between the samples. FIG. 3J shows expression of a gene of interest in an untreated cochlear explant (left panel) and after transduction with an AAV encoding a FLAG-tagged gene of interest without a microRNA target site (right panel). Immunostaining of the FLAG tag is shown in green. Immunostaining of MYO7A was used to label hair cells in red. White arrowheads indicate hair cells expressing the Connexin 26-FLAG. FIG. 3K shows a cochlear explant transduced with AAVAnc80-CAG-GOI.miRTS1 comprising a FLAG-tagged gene of interest and a microRNA targeting site for a microRNA expressed in hair cells. FIG. 3L shows a cochlear explant transduced with AAVAnc80-CAG-GOI.miRTS1 comprising a FLAG-tagged gene of interest and a microRNA targeting site for a microRNA expressed in hair cells. FIG. 3M shows a cochlear explant transduced with AAVAnc80-CAG-GOI.miRTS2 comprising FLAG-tagged gene of interest and a microRNA targeting site recognized by a microRNA expressed in hair cells. FIG. 3N shows a cochlear explant transduced with AAVAnc80-CAG-GOI.miRTS3 comprising FLAG-tagged gene of interest and a microRNA targeting site recognized by a microRNA expressed in hair cells. FIG. 3O shows a cochlear explant transduced with AAVAnc80-CAG-GOI.miRTS4 comprising a FLAG-tagged gene of interest and a microRNA targeting for a microRNA expressed in hair cells. FIGS. 3P and 3Q depict in vitro expression of GJB2 protein in HEK293FT cells transfected with CAG.5UTR.hGJB2.FLAG.miRTS.3UTR (SEQ ID NO: 87), CAG.5UTR.hGJB2.FLAG.3UTR (SEQ ID NO: 82), or CAG.5UTR.hGJB2.FLAG.GFP constructs. CAG.5UTR.hGJB2.FLAG.miRTS.3UTR comprises miRNA targeting sites (miRTS) for miR-182 and miR-183 in the 3UTR to permit exogenous hGJB2 knockdown in the presence of regulatory miR-182 and/or miR-183. To confirm miRNA regulation of constructs, HEK293FT cells were transfected with hGJB2 comprising plasmids and optionally co-transfected with (+) or without (−) plasmids expressing miR-182 and miR-183. 72 h post transfection the cells were harvested for protein and RNA analysis. FIG. 3P depicts exemplary GJB2 protein levels analyzed using western blot. FIG. 3Q depicts exemplary GJB2 mRNA levels analyzed using qPCR.

FIGS. 4A-4C depicts FLAG protein expression in mouse cochlear explants transduced at P2 with exemplary rAAVAnc80 particles comprising constructs driven by CAG, CMVe-GJB2p, or smCBA promoter/enhancer sequences as noted, explants were fixed after 72 h, immunostaining for FLAG is noted in green, immunostaining for hair cell marker Myo7a is noted in red, and nuclear marker DAPI is noted in blue. FIG. 4A, panel (A) depicts exemplary explants transduced with AAVAnc80-CAG.5UTR.hGJB2.3F.3UTR (SEQ ID NO: 82) at 5.8E9 vg/explant. FIG. 4B, panel (B)) depicts exemplary explants transduced with AAVAnc80-smCBA.5UTR.hGJB2.3F.3UTR (SEQ ID NO: 83) at 1.4E10 vg/explant. FIG. 4C, panel (C) depicts exemplary explants transduced with AAVAnc80-CMVeGFAPp.5UTR.hGJB2.3F.3UTR (SEQ ID NO: 84) at 1.8E10 vg/explant.

FIG. 5 illustrates a perspective of a device for delivering fluid to an inner ear, according to aspects of the present disclosure.

FIG. 6 illustrates a sideview of a bent needle sub-assembly, according to aspects of the present disclosure.

FIG. 7 illustrates a perspective view of a device for delivering fluid to an inner ear, according to aspects of the present disclosure.

FIG. 8 illustrates a perspective view of a bent needle sub-assembly coupled to the distal end of a device, according to aspects of the present disclosure.

FIGS. 9A-9O depicts in vivo expression of Connexin 26 in wild-type mice. Wild type mice (p20) were administered rAAVAnc80 particles comprising CAG.hGJB2.FLAG.GFP (schematic provided in FIG. 2H) to the cochlea (FIG. 9A). Expression of Connexin 26 in the supporting cells and inner hair cells was detected 10 days after administration. Immunostaining of actin filaments and hair cell stereocilia bundles by phalloidin is noted in blue, GFP is noted in green, FLAG is noted in purple, and endogenous Connexin 26 is noted in red. SC—supporting cells; IHC—inner hair cells; OHC—outer hair cells. Juvenile mice were administered rAAVAnc80 particles comprising AAVAnc80-CMVeGFAPp.mGJB2p.5UTR.hGJB2.FLAG.3UTR (FIG. 9B), AAVAnc80-GDF6p.mGJB2p.5UTR.hGJB2.FLAG.3UTR (FIGS. 9C and 9I) (schematic provided in FIG. 2G), AAVAnc80-IGFBP2p. mGJB2p.5UTR.hGJB2.FLAG.3UTR (FIG. 9D) (schematic provided in FIG. 2G), AAVAnc80-PARM1p.mGJB2p.5UTR.hGJB2.FLAG.3UTR (FIGS. 9E and 9J) (schematic provided in FIG. 2G), AAVAnc80-GFAPp.mGJB2p.hGJB2 (FIG. 9F), AAVAnc80-MMP15p.mGJB2p.hGJB2 (FIGS. 9G and 9L), AAVAnc80-VIMp.mGJB2p-hGJB2 (FIGS. 9H and 9K) to the cochlea. (VIM is also referred to as VIM1 in FIG. 9K.) Expression of Connexin 26 was detected two weeks after administration. Immunostaining of actin filaments and hair cell stereocilia bundles by phalloidin is noted in blue, FLAG is noted in green, and endogenous Connexin 26 or Myo7a is noted in red. FIG. 9M depicts in vivo expression of Connexin 26 in wild-type mice administered AAVAnc80 particles comprising AAVAnc80.CMVe.GFAP.mGJB2p.hGJB2.FLAG. Endogenous Connexin 26 is shown in white, flag-tagged Connexin 26 is shown in green, and hair cells are shown by phalloidin staining in blue. FIGS. 9N-9O) depicts in vivo expression of Connexin 26 in wild-type mice administered AAVAnc80 particles comprising AAVAnc80.CMVe.GDF6.mGJB2p.hGJB2.FLAG or AAVAnc80.CMVe.PARM1.mGJB2p.hGJB2.FLAG. Flag-tagged Connexin 26 is shown in green, phalloidin staining in blue, and Myo7a marking hair cells is shown in red.

FIGS. 10A-10C depicts in vitro expression of GJB2 mRNA and detection of Connexin 26 protein from constructs including supporting cell selective promoters. FIG. 10A shows Connexin 26-FLAG protein levels (“GJB2-FLAG”) in HEK293FT cells transduced with exemplary rAAVAnc80 particles comprising constructs driven by GJB6, IGFBP2, RPB7, PARM1, or GDF6 promoters in combination with a minimal GJB2 promoter. GAPDH is shown as a loading control. FIG. 10B shows GJB2 mRNA levels in HEK293FT cells transduced with rAAVAnc80 particles comprising constructs driven by GFAP and a minimal GJB2 promoter, CMV enhancer/GFAP, GJB2 enhancer/GJB2, CMV enhancer/GJB2, or CAG promoters. FIG. 10C shows Connexin 26-FLAG protein levels (GJB2-FLAG) in HEK293FT cells transfected with plasmids comprising constructs driven by FABP3, KLHL14, DBI2, TSPAN8, MMP15, SPARC, or VIM promoters in combination with a minimal GJB2 promoter. FLAG was used to distinguish protein levels between endogenous and transduced Connexin 26 expression. GAPDH is shown as a loading control.

FIG. 11 shows GJB2 mRNA levels in mouse cochlear explants transduced with rAAVAnc80 particles comprising constructs driven by a CAG promoter, a CMV enhancer/GFAP promoter, or a GFAP and a minimal GJB2 promoter. GJB2 mRNA levels were determined by qPCR.

### Definitions

The scope of the present disclosure is defined by the claims appended hereto and is not limited by certain aspects described herein. Those skilled in the art, reading the present specification, will be aware of various modifications that may be equivalent to such described aspects, or otherwise within the scope of the claims. In general, terms used herein are in accordance with their understood meaning in the art, unless clearly indicated otherwise. Explicit definitions of certain terms are provided below; meanings of these and other terms in particular instances throughout this specification will be clear to those skilled in the art from context.

Use of ordinal terms such as “first,” “second,” “third,” etc., in the claims to modify a claim element does not by itself connote any priority, precedence, or order of one claim element over another or the temporal order in which acts of a method are performed, but are used merely as labels to distinguish one claim element having a certain name from another element having a same name (but for use of the ordinal term) to distinguish the claim elements.

The articles “a” and “an,” as used herein, should be understood to include the plural referents unless clearly indicated to the contrary. Claims or descriptions that include “or” between one or more members of a group are considered satisfied if one, more than one, or all of the group members are present in, employed in, or otherwise relevant to a given product or process unless indicated to the contrary or otherwise evident from the context. In some aspects, exactly one member of a group is present in, employed in, or otherwise relevant to a given product or process. In some aspects, more than one, or all group members are present in, employed in, or otherwise relevant to a given product or process. It is to be understood that the present disclosure encompasses all variations, combinations, and permutations in which one or more limitations, elements, clauses, descriptive terms, etc., from one or more of the listed claims is introduced into another claim dependent on the same base claim (or, as relevant, any other claim) unless otherwise indicated or unless it would be evident to one of ordinary skill in the art that a contradiction or inconsistency would arise. Where elements are presented as lists (e.g., in Markush group or similar format), it is to be understood that each subgroup of the elements is also disclosed, and any element(s) can be removed from the group. It should be understood that, in general, where aspects or aspects are referred to as “comprising” particular elements, features, etc., certain aspects or aspects “consist,” or “consist essentially of,” such elements, features, etc. For purposes of simplicity, those aspects have not in every case been specifically set forth in so many words herein. It should also be understood that any embodiment or aspect can be explicitly excluded from the claims, regardless of whether the specific exclusion is recited in the specification.

Throughout the specification, whenever a polynucleotide or polypeptide is represented by a sequence of letters (e.g., A, C, G, and T, which denote adenosine, cytidine, guanosine, and thymidine, respectively in the case of a polynucleotide), such polynucleotides or polypeptides are presented in 5′ to 3′ or N-terminus to C-terminus order, from left to right.

Administration: As used herein, the term “administration” typically refers to administration of a construct or composition to a subject or system to achieve delivery of an agent to a subject or system. In some aspects, an agent is, or is included in, a composition; in some aspects, an agent is generated through metabolism of a composition or one or more components thereof. Those of ordinary skill in the art will be aware of a variety of routes that may, in appropriate circumstances, be utilized for administration to a subject, for example a human. For example, in some aspects, administration may be systematic or local. In some aspects, a systematic administration can be intravenous. In some aspects, administration can be local. Local administration can involve delivery to cochlear perilymph via, e.g., injection through a round-window membrane or into scala-tympani, a scala-media injection through endolymph, perilymph and/or endolymph following canalostomy. In some aspects, administration may involve only a single dose. In some aspects, administration may involve application of a fixed number of doses. In some aspects, administration may involve dosing that is intermittent (e.g., a plurality of doses separated in time) and/or periodic (e.g., individual doses separated by a common period of time) dosing. In some aspects, administration may involve continuous dosing (e.g., perfusion) for at least a selected period of time.

Allele: As used herein, the term “allele” refers to one of two or more existing genetic variants of a specific polymorphic genomic locus.

Amelioration: As used herein, the term “amelioration” refers to prevention, reduction or palliation of a state, or improvement of a state of a subject. Amelioration may include, but does not require, complete recovery or complete prevention of a disease, disorder or condition.

Amino acid: In its broadest sense, as used herein, the term “amino acid” refers to any compound and/or substance that can be incorporated into a polypeptide chain, e.g., through formation of one or more peptide bonds. In some aspects, an amino acid has a general structure, e.g., H₂N—C(H)(R)—COOH. In some aspects, an amino acid is a naturally-occurring amino acid. In some aspects, an amino acid is a non-natural amino acid; in some aspects, an amino acid is a D-amino acid; in some aspects, an amino acid is an L-amino acid. “Standard amino acid” refers to any of the twenty standard L-amino acids commonly found in naturally occurring peptides. “Nonstandard amino acid” refers to any amino acid, other than standard amino acids, regardless of whether it is prepared synthetically or obtained from a natural source. In some aspects, an amino acid, including a carboxy- and/or amino-terminal amino acid in a polypeptide, can contain a structural modification as compared with general structure as shown above. For example, in some aspects, an amino acid may be modified by methylation, amidation, acetylation, pegylation, glycosylation, phosphorylation, and/or substitution (e.g., of an amino group, a carboxylic acid group, one or more protons, and/or a hydroxyl group) as compared with a general structure. In some aspects, such modification may, for example, alter circulating half-life of a polypeptide containing a modified amino acid as compared with one containing an otherwise identical unmodified amino acid. In some aspects, such modification does not significantly alter a relevant activity of a polypeptide containing a modified amino acid, as compared with one containing an otherwise identical unmodified amino acid.

Approximately or About: As used herein, the terms “approximately” or “about” may be applied to one or more values of interest, including a value that is similar to a stated reference value. In some aspects, the term “approximately” or “about” refers to a range of values that fall within ±10% (greater than or less than) of a stated reference value unless otherwise stated or otherwise evident from context (except where such number would exceed 100% of a possible value). For example, in some aspects, the term “approximately” or “about” may encompass a range of values that within 10%, 9%, 8%, 7%, 6%, 5%, 4%, 3%, 2%, 1%, or less of a reference value.

Associated: As used herein, the term “associated” describes two events or entities as “associated” with one another, if the presence, level and/or form of one is correlated with that of the other. For example, a particular entity (e.g., polypeptide, genetic signature, metabolite, microbe, etc.) is considered to be associated with a particular disease, disorder, or condition, if its presence, level and/or form correlates with incidence of and/or susceptibility to the disease, disorder, or condition (e.g., across a relevant population). In some aspects, two or more entities are physically “associated” with one another if they interact, directly or indirectly, so that they are and/or remain in physical proximity with one another. In some aspects, two or more entities that are physically associated with one another are covalently linked to one another; in some aspects, two or more entities that are physically associated with one another are not covalently linked to one another but are non-covalently associated, for example by means of hydrogen bonds, van der Waals interaction, hydrophobic interactions, magnetism, and combinations thereof.

Biologically active: As used herein, the term “biologically active” refers to an observable biological effect or result achieved by an agent or entity of interest. For example, in some aspects, a specific binding interaction is a biological activity. In some aspects, modulation (e.g., induction, enhancement, or inhibition) of a biological pathway or event is a biological activity. In some aspects, presence or extent of a biological activity is assessed through detection of a direct or indirect product produced by a biological pathway or event of interest.

Cell Selective Promoter: As used herein, the term “cell selective promoter” refers to a promoter that is predominately active in certain cell types (e.g., transcription of a specific gene occurs only within cells expressing transcription regulatory and/or control proteins that bind to the tissue-specific promoter). In some aspects, an inner ear supporting cell selective promoter is a promoter that is predominately active in one or more supporting cells of the inner ear.

Characteristic portion: As used herein, the term “characteristic portion,” in the broadest sense, refers to a portion of a substance whose presence (or absence) correlates with presence (or absence) of a particular feature, attribute, or activity of the substance. In some aspects, a characteristic portion of a substance is a portion that is found in a given substance and in related substances that share a particular feature, attribute or activity, but not in those that do not share the particular feature, attribute or activity. In some aspects, a characteristic portion shares at least one functional characteristic with the intact substance. For example, in some aspects, a “characteristic portion” of a protein or polypeptide is one that contains a continuous stretch of amino acids, or a collection of continuous stretches of amino acids, that together are characteristic of a protein or polypeptide. In some aspects, each such continuous stretch generally contains at least 2, 5, 10, 15, 20, 50, or more amino acids. In general, a characteristic portion of a substance (e.g., of a protein, antibody, etc.) is one that, in addition to a sequence and/or structural identity specified above, shares at least one functional characteristic with the relevant intact substance. In some aspects, a characteristic portion may be biologically active.

Characteristic sequence: As used herein, the term “characteristic sequence” is a sequence that is found in all members of a family of polypeptides or nucleic acids, and therefore can be used by those of ordinary skill in the art to define members of the family.

Characteristic sequence element: As used herein, the phrase “characteristic sequence element” refers to a sequence element found in a polymer (e.g., in a polypeptide or nucleic acid) that represents a characteristic portion of that polymer. In some aspects, presence of a characteristic sequence element correlates with presence or level of a particular activity or property of a polymer. In some aspects, presence (or absence) of a characteristic sequence element defines a particular polymer as a member (or not a member) of a particular family or group of such polymers. A characteristic sequence element typically comprises at least two monomers (e.g., amino acids or nucleotides). In some aspects, a characteristic sequence element includes at least 2, 3, 4, 5, 6, 7, 8, 9, 10, 11, 12, 13, 14, 15, 20, 25, 30, 35, 40, 45, 50, or more monomers (e.g., contiguously linked monomers). In some aspects, a characteristic sequence element includes at least first and second stretches of contiguous monomers spaced apart by one or more spacer regions whose length may or may not vary across polymers that share a sequence element.

Combination therapy: As used herein, the term “combination therapy” refers to those situations in which a subject is simultaneously exposed to two or more therapeutic regimens (e.g., two or more therapeutic agents). In some aspects, two or more agents may be administered simultaneously. In some aspects, two or more agents may be administered sequentially. In some aspects, two or more agents may be administered in overlapping dosing regimens.

Comparable: As used herein, the term “comparable” refers to two or more agents, entities, situations, sets of conditions, subjects, populations, etc., that may not be identical to one another but that are sufficiently similar to permit comparison therebetween so that one skilled in the art will appreciate that conclusions may reasonably be drawn based on differences or similarities observed. In some aspects, comparable sets of agents, entities, situations, sets of conditions, subjects, populations, etc. are characterized by a plurality of substantially identical features and one or a small number of varied features. Those of ordinary skill in the art will understand, in context, what degree of identity is required in any given circumstance for two or more such agents, entities, situations, sets of conditions, subjects, populations, etc. to be considered comparable. For example, those of ordinary skill in the art will appreciate that sets of agents, entities, situations, sets of conditions, subjects, populations, etc. are comparable to one another when characterized by a sufficient number and type of substantially identical features to warrant a reasonable conclusion that differences in results obtained or phenomena observed under or with different sets of circumstances, stimuli, agents, entities, situations, sets of conditions, subjects, populations, etc. are caused by or indicative of the variation in those features that are varied.

Construct: As used herein, the term “construct” refers to a composition including a polynucleotide capable of carrying at least one heterologous polynucleotide. In some aspects, a construct can be a plasmid, a transposon, a cosmid, an artificial chromosome (e.g., a human artificial chromosome (HAC), a yeast artificial chromosome (YAC), a bacterial artificial chromosome (BAC), or a P1-derived artificial chromosome (PAC)) or a viral vector, capsid, viral particle and any Gateway® plasmids. A construct can, e.g., include sufficient cis-acting elements for expression; other elements for expression can be supplied by the host primate cell or in an in vitro expression system. A construct may include any genetic element (e.g., a plasmid, a transposon, a cosmid, an artificial chromosome, or a viral vector, capsid, viral particle etc.) that is capable of replicating when associated with proper control elements. Thus, in some aspects, “construct” may include a cloning and/or expression construct and/or a viral construct (e.g., an adeno-associated virus (AAV) construct, an adenovirus construct, a lentivirus construct, or a retrovirus construct).

Conservative: As used herein, the term “conservative” refers to instances describing a conservative amino acid substitution, including a substitution of an amino acid residue by another amino acid residue having a side chain R group with similar chemical properties (e.g., charge or hydrophobicity). In general, a conservative amino acid substitution will not substantially change functional properties of interest of a protein, for example, ability of a receptor to bind to a ligand. Examples of groups of amino acids that have side chains with similar chemical properties include: aliphatic side chains such as glycine (Gly, G), alanine (Ala, A), valine (Val, V), leucine (Leu, L), and isoleucine (Ile, I); aliphatic-hydroxyl side chains such as serine (Ser, S) and threonine (Thr, T); amide-containing side chains such as asparagine (Asn, N) and glutamine (Gln, Q); aromatic side chains such as phenylalanine (Phe, F), tyrosine (Tyr, Y), and tryptophan (Trp, W); basic side chains such as lysine (Lys, K), arginine (Arg, R), and histidine (His, H); acidic side chains such as aspartic acid (Asp, D) and glutamic acid (Glu, E); and sulfur-containing side chains such as cysteine (Cys, C) and methionine (Met, M). Conservative amino acids substitution groups include, for example, valine/leucine/isoleucine (Val/Leu/Ile, V/L/I), phenylalanine/tyrosine (Phe/Tyr, F/Y), lysine/arginine (Lys/Arg, K/R), alanine/valine (Ala/Val, A/V), glutamate/aspartate (Glu/Asp, E/D), and asparagine/glutamine (Asn/Gln, N/Q). In some aspects, a conservative amino acid substitution can be a substitution of any native residue in a protein with alanine, as used in, for example, alanine scanning mutagenesis. In some aspects, a conservative substitution is made that has a positive value in the PAM250 log-likelihood matrix disclosed in Gonnet et al., 1992, Science 256:1443-1445, which is incorporated herein by reference in its entirety. In some aspects, a substitution is a moderately conservative substitution wherein the substitution has a nonnegative value in the PAM250 log-likelihood matrix. One skilled in the art would appreciate that a change (e.g., substitution, addition, deletion, etc.) of amino acids that are not conserved between the same protein from different species is less likely to have an effect on the function of a protein and therefore, these amino acids should be selected for mutation. Amino acids that are conserved between the same protein from different species should not be changed (e.g., deleted, added, substituted, etc.), as these mutations are more likely to result in a change in function of a protein. Exemplary conservative amino acid substitutions are shown in Table 1.

TABLE 1
Conservative Amino Acid Substitutions
CONSERVATIVE AMINO ACID SUBSTITUTIONS

| For | | |
|---|---|---|
| Amino | | |
| Acid | Code | Replace With |
| Alanine | A | D-ala, Gly, Aib, β-Ala, Acp, L-Cys, D-Cys |
| Arginine | R | D-Arg, Lys, D-Lys, homo-Arg, D-homo-Arg, Met, |
| | | Ile, D-Met, D-Ile, Orn, D-Orn |
| Asparagine | N | D-Asn, Asp, D-Asp, Glu, D-Glu, Gln, D-Gln |
| Aspartic | D | D-Asp, D-Asn, Asn, Glu, D-Glu, Gln, D-Gln |
| Acid | | |
| Cysteine | C | D-Cys, S-Me-Cys, Met, D-Met, Thr, D-Thr |
| Glutamine | Q | D-Gln, Asn, D-Asn, Glu, D-Glu, Asp, D-Asp |
| Glutamic | E | D-Glu, D-Asp, Asp, Asn, D-Asn, Gln, D-Gln |
| Acid | | |
| Glycine | G | Ala, D-Ala, Pro, D-Pro, Aib, β-Ala, Acp |
| Isoleucine | I | D-Ile, Val, D-Val, AdaA, AdaG, Leu, D-Leu, Met, |
| | | D-Met |
| Leucine | L | D-Leu, Val, D-Val, AdaA, AdaG, Leu, D-Leu, Met, |
| | | D-Met |
| Lysine | K | D-Lys, Arg, D-Arg, homo-Arg, D-homo-Arg, Met, |
| | | D-Met, Ile, D-Ile, Orn, D-Orn |
| Methionine | M | D-Met, S-Me-Cys, Ile, D-Ile, Leu, D-Leu, Val, D- |
| | | Val |
| Phenyl- | F | D-Phe, Tyr, D-Thr, L-Dopa, His, D-His, Trp, D- |
| alanine | | Trp, Trans-3,4 or 5-phenylproline, AdaA, AdaG, |
| | | cis-3,4 or 5-phenylproline, Bpa, D-Bpa |
| Proline | P | D-Pro, L-I-thioazolidine-4-carboxylic acid, D-or-L- |
| | | 1-oxazolidine-4-carboxylic acid (Kauer, U.S. Pat. |
| | | No. 4,511,390) |
| Serine | S | D-Ser, Thr, D-Thr, allo-Thr, Met, D-Met, Met (O), |
| | | D-Met (O), L-Cys, D-Cys |
| Threonine | T | D-Thr, Ser, D-Ser, allo-Thr, Met, D-Met, Met (O), |
| | | D-Met (O), Val, D-Val |
| Tyrosine | Y | D-Tyr, Phe, D-Phe, L-Dopa, His, D-His |
| Valine | V | D-Val, Leu, D-Leu, Ile, D-Ile, Met, D-Met, AdaA, |
| | | AdaG |

Control: As used herein, the term “control” refers to the art-understood meaning of a “control” being a standard against which results are compared. Typically, controls are used to augment integrity in experiments by isolating variables in order to make a conclusion about such variables. In some aspects, a control is a reaction or assay that is performed simultaneously with a test reaction or assay to provide a comparator. For example, in one experiment, a “test” (i.e., a variable being tested) is applied. In a second experiment, a “control,” the variable being tested is not applied. In some aspects, a control is a historical control (e.g., of a test or assay performed previously, or an amount or result that is previously known). In some aspects, a control is or comprises a printed or otherwise saved record. In some aspects, a control is a positive control. In some aspects, a control is a negative control.

Determining, measuring, evaluating, assessing, assaying and analyzing: As used herein, the terms “determining,” “measuring,” “evaluating,” “assessing,” “assaying,” and “analyzing” may be used interchangeably to refer to any form of measurement, and include determining if an element is present or not. These terms include both quantitative and/or qualitative determinations. Assaying may be relative or absolute. For example, in some aspects, “Assaying for the presence of” can be determining an amount of something present and/or determining whether or not it is present or absent.

Endogenous: As used herein in reference to a substances or process refers to a naturally occuring substances or processes that originates from within a system such as an organism, tissue, or cell.

Engineered: In general, as used herein, the term “engineered” refers to an aspect of having been manipulated by the hand of man. For example, a cell or organism is considered to be “engineered” if it has been manipulated so that its genetic information is altered (e.g., new genetic material not previously present has been introduced, for example by transformation, mating, somatic hybridization, transfection, transduction, or other mechanism, or previously present genetic material is altered or removed, for example by substitution or deletion mutation, or by mating protocols). As is common practice and is understood by those in the art, progeny of an engineered polynucleotide or cell are typically still referred to as “engineered” even though the actual manipulation was performed on a prior entity.

Excipient: As used herein, the term “excipient” refers to an inactive (e.g., non-therapeutic) agent that may be included in a pharmaceutical composition, for example to provide or contribute to a desired consistency or stabilizing effect. In some aspects, suitable pharmaceutical excipients may include, for example, starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like.

Expression: As used herein, the term “expression” of a nucleic acid sequence refers to generation of any gene product (e.g., transcript, e.g., mRNA, e.g., polypeptide, etc.) from a nucleic acid sequence. In some aspects, a gene product can be a transcript. In some aspects, a gene product can be a polypeptide. In some aspects, expression of a nucleic acid sequence involves one or more of the following: (1) production of an RNA template from a DNA sequence (e.g., by transcription); (2) processing of an RNA transcript (e.g., by splicing, editing, 5′ cap formation, and/or 3′ end formation); (3) translation of an RNA into a polypeptide or protein; and/or (4) post-translational modification of a polypeptide or protein.

Flanked: As used herein, the term “flanked” refers to a position relative to ends of a reference item. More specifically, in referring to reference nucleic acid sequence(s), “flanked” refers to having a sequences upstream and downstream of the reference nucleic acid sequence(s). In some aspects, a flanked referenced nucleic acid sequence has a first sequence or series of nucleotide residues positioned adjacent to the 5′ end of the referenced nucleic acid and a second sequence or series of nucleotide residues positioned adjacent to the 3′ end of the referenced nucleic acid. In some aspects, the upstream and/or downstream flanking sequences are immediately adjacent to the referenced nucleic acid sequence. In some aspects, there are intervening nucleic acids between the upstream and/or downstream flanking sequences and the referenced nucleic acid sequence.

Functional: As used herein, the term “functional” describes something that exists in a form in which it exhibits a property and/or activity by which it is characterized. For example, in some aspects, a “functional” biological molecule is a biological molecule in a form in which it exhibits a property and/or activity by which it is characterized. In some such aspects, a functional biological molecule is characterized relative to another biological molecule which is non-functional in that the “non-functional” version does not exhibit the same or equivalent property and/or activity as the “functional” molecule. A biological molecule may have one function, two functions (i.e., bifunctional) or many functions (i.e., multifunctional).

Gene: As used herein, the term “gene” refers to a DNA sequence in a chromosome that codes for a gene product (e.g., an RNA product, e.g., a polypeptide product). In some aspects, a gene includes coding sequence (i.e., sequence that encodes a particular product). In some aspects, a gene includes non-coding sequence. In some particular aspects, a gene may include both coding (e.g., exonic) and non-coding (e.g., intronic) sequence. In some aspects, a gene may include one or more regulatory sequences (e.g., promoters, enhancers, etc.) and/or intron sequences that, for example, may control or impact one or more aspects of gene expression (e.g., cell-type-specific expression, inducible expression, etc.). As used herein, the term “gene” generally refers to a portion of a nucleic acid that encodes a polypeptide or fragment thereof; the term may optionally encompass regulatory sequences, as will be clear from context to those of ordinary skill in the art. This definition is not intended to exclude application of the term “gene” to non-protein-coding expression units but rather to clarify that, in most cases, the term as used in this document refers to a polypeptide-coding nucleic acid. In some aspects, a gene may encode a polypeptide, but that polypeptide may not be functional, e.g., a gene variant may encode a polypeptide that does not function in the same way, or at all, relative to the wild-type gene. In some aspects, a gene may encode a transcript which, in some aspects, may be toxic beyond a threshold level. In some aspects, a gene may encode a polypeptide, but that polypeptide may not be functional and/or may be toxic beyond a threshold level.

Hearing loss: As used herein, the term “hearing loss” may be used to a partial or total inability of a living organism to hear. In some aspects, hearing loss may be acquired. In some aspects, hearing loss may be hereditary. In some aspects, hearing loss may be genetic. In some aspects, hearing loss may be as a result of disease or trauma (e.g., physical trauma, treatment with one or more agents resulting in hearing loss, etc.). In some aspects, hearing loss may be due to one or more known genetic causes and/or syndromes. In some aspects, hearing loss may be of unknown etiology. In some aspects, hearing loss may or may not be mitigated by use of hearing aids or other treatments.

Heterologous: As used herein, the term “heterologous” the relationship between two or more nucleic acid or protein sequences that are derived from different sources. In some aspects, the promoter operably linked to the nucleic acid encoding the therapeutic protein may be derived from a different gene other than the gene encoding the therapeutic protein.

Identity: As used herein, the term “identity” refers to overall relatedness between polymeric molecules, e.g., between nucleic acid molecules (e.g., DNA molecules and/or RNA molecules) and/or between polypeptide molecules. In some aspects, polymeric molecules are considered to be “substantially identical” to one another if their sequences are at least 25%, 30%, 35%, 40%, 45%, 50%, 55%, 60%, 65%, 70%, 75%, 80%, 85%, 90%, 95%, or 99% identical. Calculation of percent identity of two nucleic acid or polypeptide sequences, for example, can be performed by aligning two sequences for optimal comparison purposes (e.g., gaps can be introduced in one or both of a first and a second sequences for optimal alignment and non-identical sequences can be disregarded for comparison purposes). In some aspects, a length of a sequence aligned for comparison purposes is at least 30%, at least 40%, at least 50%, at least 60%, at least 70%, at least 80%, at least 90%, at least 95%, or substantially 100% of length of a reference sequence; nucleotides at corresponding positions are then compared. When a position in the first sequence is occupied by the same residue (e.g., nucleotide or amino acid) as a corresponding position in the second sequence, then the two molecules (i.e., first and second) are identical at that position. Percent identity between two sequences is a function of the number of identical positions shared by the two sequences being compared, taking into account the number of gaps, and the length of each gap, which needs to be introduced for optimal alignment of the two sequences. Comparison of sequences and determination of percent identity between two sequences can be accomplished using a mathematical algorithm. For example, percent identity between two nucleotide sequences can be determined using the algorithm of Meyers and Miller (CABIOS, 1989, 4: 11-17, which is herein incorporated by reference in its entirety), which has been incorporated into the ALIGN program (version 2.0). In some aspects, nucleic acid sequence comparisons made with the ALIGN program use a PAM120 weight residue table, a gap length penalty of 12 and a gap penalty of 4.

Improve, increase, enhance, inhibit or reduce: As used herein, the terms “improve,” “increase,” “enhance,” “inhibit,” “reduce,” or grammatical equivalents thereof, indicate values that are relative to a baseline or other reference measurement. In some aspects, a value is statistically significantly difference that a baseline or other reference measurement. In some aspects, an appropriate reference measurement may be or comprise a measurement in a particular system (e.g., in a single individual) under otherwise comparable conditions absent presence of (e.g., prior to and/or after) a particular agent or treatment, or in presence of an appropriate comparable reference agent. In some aspects, an appropriate reference measurement may be or comprise a measurement in comparable system known or expected to respond in a particular way, in presence of the relevant agent or treatment. In some aspects, an appropriate reference is a negative reference; in some aspects, an appropriate reference is a positive reference.

Knockdown: As used herein, the term “knockdown” refers to a decrease in expression of one or more gene products. In some aspects, an inhibitory nucleic acid achieve knockdown. In some aspects, a genome editing system described herein achieves knockdown.

Knockout: As used herein, the term “knockout” refers to ablation of expression of one or more gene products. In some aspects, a genome editing system described herein achieve knockout.

Minimal Promoter: As used herein, the term “minimal promoter”, unless indicated otherwise, refers to a promoter that includes less than the full naturally occurring promoter sequence, which is still capable of directing transcription of a coding sequence (e.g., a heterogenous or homogenous coding sequence).

In some aspects, the minimal promoter can comprise one or more regions (including all regions) of the fully naturally occurring promoter that can direct transcription of a coding sequence.

In some aspects, the minimal promoter can comprise a portion or portions of the region(s) of the fully naturally occurring promoter that can direct transcription of a coding sequence.

microRNA: As used herein, the term “microRNA” or “miRNA” refers to a class of biomolecules involved in control of gene expression. A mature miRNA is typically an 18-25 nucleotide non-coding RNA that regulates expression of an mRNA including sequences complementary to the miRNA. These small RNA molecules are known to control gene expression by regulating the stability and/or translation of mRNAs. For example, miRNAs bind to the 3′ UTR of target mRNAs and suppress translation. MiRNAs may also bind to target mRNAs and mediate gene silencing through the RNAi pathway. MiRNAs may also regulate gene expression by causing chromatin condensation.

In some aspects, a microRNA is between about 10 nucleotides to about 30 nucleotides in length (e.g., about 10 nucleotides to about 28 nucleotides, about 10 nucleotides to about 26 nucleotides, about 10 nucleotides to about 24 nucleotides, about 10 nucleotides to about 22 nucleotides, about 10 nucleotides to about 20 nucleotides, about 10 nucleotides to about 18 nucleotides, about 10 nucleotides to about 16 nucleotides, about 10 nucleotides to about 14 nucleotides, about 10 nucleotides to about 12 nucleotides, about 12 nucleotides to about 30 nucleotides, about 12 nucleotides to about 28 nucleotides, about 12 nucleotides to about 26 nucleotides, about 12 nucleotides to about 24 nucleotides, about 12 nucleotides to about 22 nucleotides, about 12 nucleotides to about 20 nucleotides, about 12 nucleotides to about 18 nucleotides, about 12 nucleotides to about 16 nucleotides, about 12 nucleotides to about 14 nucleotides, about 16 nucleotides to about 30 nucleotides, about 16 nucleotides to about 28 nucleotides, about 16 nucleotides to about 26 nucleotides, about 16 nucleotides to about 24 nucleotides, about 16 nucleotides to about 22 nucleotides, about 16 nucleotides to about 20 nucleotides, about 16 nucleotides to about 18 nucleotides, about 18 nucleotides to about 30 nucleotides, about 18 nucleotides to about 28 nucleotides, about 18 nucleotides to about 26 nucleotides, about 18 nucleotides to about 24 nucleotides, about 18 nucleotides to about 22 nucleotides, about 18 nucleotides to about 20 nucleotides, about 20 nucleotides to about 30 nucleotides, about 20 nucleotides to about 28 nucleotides, about 20 nucleotides to about 26 nucleotides, about 20 nucleotides to about 24 nucleotides, about 20 nucleotides to about 22 nucleotides, about 22 nucleotides to about 30 nucleotides, about 22 nucleotides to about 28 nucleotides, about 22 nucleotides to about 26 nucleotides, about 22 nucleotides to about 24 nucleotides, about 24 nucleotides to about 30 nucleotides, about 24 nucleotides to about 28 nucleotides, about 24 nucleotides to about 26 nucleotides, about 26 nucleotides to about 30 nucleotides, about 26 nucleotides to about 28 nucleotides, about 28 nucleotides to about 30 nucleotides, or 11, 12, 13, 14, 15, 16, 17, 18, 19, 20, 21, 22, 23, 24, 25, 26, 27, 28, 29 or 30 nucleotides).

microRNA regulatory target site: As used herein, the term “microRNA regulatory target site” or “miRTS” refers to a sequence that directly interacts with a miRNA on the mRNA transcript. Often, the miRTS is present in the 3′ untranslated region (UTR) of the mRNA, but it may also be present in the coding sequence, or in the 5′ UTR. miRTS are not necessarily perfect complements to miRNAs, usually having only a few bases of complementarity to the miRNA, and often containing one or more mismatches. The miRTS may be any sequence capable of being bound by a miRNA sufficiently that the translation of a gene to which the miRTS is operably linked is repressed by a miRNA silencing mechanism such as the RISC. In some aspects, inclusion of a miRTS into a nucleic acid construct comprising a polynucleotide (e.g., a therapeutic polynucleotide) can result in degradation of the therapeutic polynucleotide after transcription.

Nucleic acid: As used herein, the term “nucleic acid”, in its broadest sense, refers to any compound and/or substance that is or can be incorporated into an oligonucleotide chain. In some aspects, a nucleic acid is a compound and/or substance that is or can be incorporated into an oligonucleotide chain via a phosphodiester linkage. As will be clear from context, in some aspects, “nucleic acid” refers to an individual nucleic acid residue (e.g., a nucleotide and/or nucleoside); in some aspects, “nucleic acid” refers to an oligonucleotide chain comprising individual nucleic acid residues. In some aspects, a “nucleic acid” is or comprises RNA; in some aspects, a “nucleic acid” is or comprises DNA. In some aspects, a nucleic acid is, comprises, or consists of one or more natural nucleic acid residues. In some aspects, a nucleic acid is, comprises, or consists of one or more nucleic acid analogs. In some aspects, a nucleic acid analog differs from a nucleic acid in that it does not utilize a phosphodiester backbone. Alternatively or additionally, in some aspects, a nucleic acid has one or more phosphorothioate and/or 5′-N-phosphoramidite linkages rather than phosphodiester bonds. In some aspects, a nucleic acid is, comprises, or consists of one or more natural nucleosides (e.g., adenosine, thymidine, guanosine, cytidine, uridine, deoxyadenosine, deoxythymidine, deoxy guanosine, and deoxycytidine). In some aspects, a nucleic acid is, comprises, or consists of one or more nucleoside analogs (e.g., 2-aminoadenosine, 2-thiothymidine, inosine, pyrrolo-pyrimidine, 3-methyl adenosine, 5-methylcytidine, C-5 propynyl-cytidine, C-5 propynyl-uridine, 2-aminoadenosine, C5-bromouridine, C5-fluorouridine, C5-iodouridine, C5-propynyl-uridine, C5-propynyl-cytidine, C5-methylcytidine, 2-aminoadenosine, 7-deazaadenosine, 7-deazaguanosine, 8-oxoadenosine, 8-oxoguanosine, 0(6)-methylguanine, 2-thiocytidine, methylated bases, intercalated bases, and combinations thereof). In some aspects, a nucleic acid comprises one or more modified sugars (e.g., 2′-fluororibose, ribose, 2′-deoxyribose, arabinose, and hexose) as compared with those in natural nucleic acids. In some aspects, a nucleic acid has a nucleotide sequence that encodes a functional gene product such as an RNA or protein. In some aspects, a nucleic acid includes one or more introns. In some aspects, nucleic acids are prepared by one or more of isolation from a natural source, enzymatic synthesis by polymerization based on a complementary template (in vivo or in vitro), reproduction in a recombinant cell or system, and chemical synthesis. In some aspects, a nucleic acid is at least 3, 4, 5, 6, 7, 8, 9, 10, 15, 20, 25, 30, 35, 40, 45, 50, 55, 60, 65, 70, 75, 80, 85, 90, 95, 100, 1 10, 120, 130, 140, 150, 160, 170, 180, 190, 20, 225, 250, 275, 300, 325, 350, 375, 400, 425, 450, 475, 500, 600, 700, 800, 900, 1000, 1500, 2000, 2500, 3000, 3500, 4000, 4500, 5000 or more residues long. In some aspects, a nucleic acid is partly or wholly single stranded; in some aspects, a nucleic acid is partly or wholly double stranded. In some aspects, a nucleic acid has a nucleotide sequence comprising at least one element that encodes, or is complementary to a sequence that encodes, a polypeptide. In some aspects, a nucleic acid has enzymatic activity.

Operably linked: As used herein, refers to a juxtaposition wherein the components described are in a relationship permitting them to function in their intended manner. A control element “operably linked” to a functional element is associated in such a way that expression and/or activity of the functional element is achieved under conditions compatible with the control element. In some aspects, “operably linked” control elements are contiguous (e.g., covalently linked) with coding elements of interest; in some aspects, control elements act in trans to or otherwise at a from the functional element of interest. In some aspects, “operably linked” refers to functional linkage between a regulatory sequence and a heterologous nucleic acid sequence resulting in expression of the latter. For example, a first nucleic acid sequence is operably linked with a second nucleic acid sequence when the first nucleic acid sequence is placed in a functional relationship with the second nucleic acid sequence. In some aspects, for example, a functional linkage may include transcriptional control. For instance, a promoter is operably linked to a coding sequence if the promoter affects the transcription or expression of the coding sequence. Operably linked DNA sequences can be contiguous with each other and, e.g., where necessary to join two protein coding regions, are in the same reading frame.

Pharmaceutical composition: As used herein, the term “pharmaceutical composition” refers to a composition in which an active agent is formulated together with one or more pharmaceutically acceptable carriers. In some aspects, an active agent is present in unit dose amount appropriate for administration in a therapeutic regimen that shows a statistically significant probability of achieving a predetermined therapeutic effect when administered to a relevant population. In some aspects, a pharmaceutical composition may be specially formulated for administration in solid or liquid form, including those adapted for, e.g., administration, for example, an injectable formulation that is, e.g., an aqueous or non-aqueous solution or suspension or a liquid drop designed to be administered into an ear canal. In some aspects, a pharmaceutical composition may be formulated for administration via injection either in a particular organ or compartment, e.g., directly into an ear, or systemic, e.g., intravenously. In some aspects, a formulation may be or comprise drenches (aqueous or non-aqueous solutions or suspensions), tablets, boluses, powders, granules, pastes, capsules, powders, etc. In some aspects, an active agent may be or comprise an isolated, purified, or pure compound.

Pharmaceutically acceptable: As used herein, the term “pharmaceutically acceptable” which, for example, may be used in reference to a carrier, diluent, or excipient used to formulate a pharmaceutical composition as disclosed herein, means that a carrier, diluent, or excipient is compatible with other ingredients of a composition and not deleterious to a recipient thereof.

Pharmaceutically acceptable carrier: As used herein, the term “pharmaceutically acceptable carrier” means a pharmaceutically-acceptable material, composition or vehicle, such as a liquid or solid filler, diluent, excipient, or solvent encapsulating material, involved in carrying or transporting a subject compound from one organ, or portion of a body, to another organ, or portion of a body. Each carrier must be is “acceptable” in the sense of being compatible with other ingredients of a formulation and not injurious to a patient. Some examples of materials which can serve as pharmaceutically-acceptable carriers include: sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; alginic acid; pyrogen-free water; isotonic saline; Ringer's solution; ethyl alcohol; pH buffered solutions; polyesters, polycarbonates and/or polyanhydrides; and other non-toxic compatible substances employed in pharmaceutical formulations.

Polyadenylation: As used herein, “polyadenylation” refers to the covalent linkage of a polyadenylyl moiety, or its modified variant, to a messenger RNA molecule. In eukaryotic organisms, most messenger RNA (mRNA) molecules are polyadenylated at the 3′ end. In some aspects, a 3′ poly(A) tail is a long sequence of adenine nucleotides (e.g., 50, 60, 70, 100, 200, 500, 1000, 2000, 3000, 4000, or 5000) added to the pre-mRNA through the action of an enzyme, polyadenylate polymerase. In higher eukaryotes, a poly(A) tail can be added onto transcripts that contain a specific sequence, the polyadenylation signal or “poly(A) sequence.” A poly(A) tail and proteins bound to it aid in protecting mRNA from degradation by exonucleases. Polyadenylation can be affect transcription termination, export of the mRNA from the nucleus, and translation. Typically, polyadenylation occurs in the nucleus immediately after transcription of DNA into RNA, but additionally can also occur later in the cytoplasm. After transcription has been terminated, the mRNA chain can be cleaved through the action of an endonuclease complex associated with RNA polymerase. The cleavage site can be characterized by the presence of the base sequence AAUAAA near the cleavage site. After mRNA has been cleaved, adenosine residues can be added to the free 3′ end at the cleavage site. As used herein, a “poly(A) sequence” is a sequence that triggers the endonuclease cleavage of an mRNA and the additional of a series of adenosines to the 3′ end of the cleaved mRNA.

Polypeptide: As used herein, the term “polypeptide” refers to any polymeric chain of residues (e.g., amino acids) that are typically linked by peptide bonds. In some aspects, a polypeptide has an amino acid sequence that occurs in nature. In some aspects, a polypeptide has an amino acid sequence that does not occur in nature. In some aspects, a polypeptide has an amino acid sequence that is engineered in that it is designed and/or produced through action of the hand of man. In some aspects, a polypeptide may comprise or consist of natural amino acids, non-natural amino acids, or both. In some aspects, a polypeptide may include one or more pendant groups or other modifications, e.g., modifying or attached to one or more amino acid side chains, at a polypeptide's N-terminus, at a polypeptide's C-terminus, or any combination thereof. In some aspects, such pendant groups or modifications may be acetylation, amidation, lipidation, methylation, pegylation, etc., including combinations thereof. In some aspects, polypeptides may contain L-amino acids, D-amino acids, or both and may contain any of a variety of amino acid modifications or analogs known in the art. In some aspects, useful modifications may be or include, e.g., terminal acetylation, amidation, methylation, etc. In some aspects, a protein may comprise natural amino acids, non-natural amino acids, synthetic amino acids, and combinations thereof. The term “peptide” is generally used to refer to a polypeptide having a length of less than about 100 amino acids, less than about 50 amino acids, less than 20 amino acids, or less than 10 amino acids. In some aspects, a polypeptide can be a therapeutic polypeptide. In some aspects, a polypeptide can be a supporting cell polypeptide. In some aspects, a polypeptide can be a reporter polypeptide.

Polynucleotide: As used herein, the term “polynucleotide” refers to any polymeric chain of nucleic acids. In some aspects, a polynucleotide is or comprises RNA; in some aspects, a polynucleotide is or comprises DNA. In some aspects, a polynucleotide is, comprises, or consists of one or more natural nucleic acid residues. In some aspects, a polynucleotide is, comprises, or consists of one or more nucleic acid analogs. In some aspects, a polynucleotide analog differs from a nucleic acid in that it does not utilize a phosphodiester backbone. Alternatively or additionally, in some aspects, a polynucleotide has one or more phosphorothioate and/or 5′-N-phosphoramidite linkages rather than phosphodiester bonds. In some aspects, a polynucleotide is, comprises, or consists of one or more natural nucleosides (e.g., adenosine, thymidine, guanosine, cytidine, uridine, deoxyadenosine, deoxythymidine, deoxy guanosine, and deoxycytidine). In some aspects, a polynucleotide is, comprises, or consists of one or more nucleoside analogs (e.g., 2-aminoadenosine, 2-thiothymidine, inosine, pyrrolo-pyrimidine, 3-methyl adenosine, 5-methylcytidine, C-5 propynyl-cytidine, C-5 propynyl-uridine, 2-aminoadenosine, C5-bromouridine, C5-fluorouridine, C5-iodouridine, C5-propynyl-uridine, C5-propynyl-cytidine, C5-methylcytidine, 2-aminoadenosine, 7-deazaadenosine, 7-deazaguanosine, 8-oxoadenosine, 8-oxoguanosine, 0(6)-methylguanine, 2-thiocytidine, methylated bases, intercalated bases, and combinations thereof). In some aspects, a polynucleotide comprises one or more modified sugars (e.g., 2′-fluororibose, ribose, 2′-deoxyribose, arabinose, and hexose) as compared with those in natural nucleic acids. In some aspects, a polynucleotide has a nucleotide sequence that encodes a functional gene product such as an RNA or protein. In some aspects, a polynucleotide includes one or more introns. In some aspects, a polynucleotide is prepared by one or more of isolation from a natural source, enzymatic synthesis by polymerization based on a complementary template (in vivo or in vitro), reproduction in a recombinant cell or system, and chemical synthesis. In some aspects, a polynucleotide is at least 3, 4, 5, 6, 7, 8, 9, 10, 15, 20, 25, 30, 35, 40, 45, 50, 55, 60, 65, 70, 75, 80, 85, 90, 95, 100, 1 10, 120, 130, 140, 150, 160, 170, 180, 190, 20, 225, 250, 275, 300, 325, 350, 375, 400, 425, 450, 475, 500, 600, 700, 800, 900, 1000, 1500, 2000, 2500, 3000, 3500, 4000, 4500, 5000 or more residues long. In some aspects, a polynucleotide is partly or wholly single stranded; in some aspects, a polynucleotide is partly or wholly double stranded. In some aspects, a polynucleotide has a nucleotide sequence comprising at least one element that encodes, or is the complement of a sequence that encodes, a polypeptide. In some aspects, a polynucleotide has enzymatic activity.

Promoter: As used herein, the term “promoter” refers to a nucleic acid sequence that functions to control the transcription of one or more coding sequences (e.g., a gene or transgene, e.g., encoding a polypeptide (e.g., a therapeutic polypeptide)), located upstream with respect to the direction of transcription of the transcription initiation site of the coding sequence. In some aspects, the promoter is structurally identified by the presence of a binding site for DNA-dependent RNA polymerase, transcription initiation sites or other DNA sequence (e.g., a transcription factor binding site, a repressor and/or activator protein binding site, or other sequences of nucleotides that act directly or indirectly to regulate the amount of transcription from the promoter). In some aspects, the promoter can comprise a naturally occurring promoter sequence, a functional fragment thereof, or a mutant of the naturally occurring promoter sequence or a functional fragment thereof.

Protein: As used herein, the term “protein” refers to a polypeptide (i.e., a string of at least two amino acids linked to one another by peptide bonds). Proteins may include moieties other than amino acids (e.g., may be glycoproteins, proteoglycans, etc.) and/or may be otherwise processed or modified. Those of ordinary skill in the art will appreciate that a “protein” can be a complete polypeptide chain as produced by a cell (with or without a signal sequence), or can be a characteristic portion thereof. Those of ordinary skill will appreciate that a protein can sometimes include more than one polypeptide chain, for example linked by one or more disulfide bonds or associated by other means.

Recombinant: As used herein, the term “recombinant” is intended to refer to polypeptides that are designed, engineered, prepared, expressed, created, manufactured, and/or or isolated by recombinant means, such as polypeptides expressed using a recombinant expression construct transfected into a host cell; polypeptides isolated from a recombinant, combinatorial human polypeptide library; polypeptides isolated from an animal (e.g., a mouse, rabbit, sheep, fish, etc.) that is transgenic for or otherwise has been manipulated to express a gene or genes, or gene components that encode and/or direct expression of the polypeptide or one or more component(s), portion(s), element(s), or domain(s) thereof; and/or polypeptides prepared, expressed, created or isolated by any other means that involves splicing or ligating selected nucleic acid sequence elements to one another, chemically synthesizing selected sequence elements, and/or otherwise generating a nucleic acid that encodes and/or directs expression of a polypeptide or one or more component(s), portion(s), element(s), or domain(s) thereof. In some aspects, one or more of such selected sequence elements is found in nature. In some aspects, one or more of such selected sequence elements is designed in silico. In some aspects, one or more such selected sequence elements results from mutagenesis (e.g., in vivo or in vitro) of a known sequence element, e.g., from a natural or synthetic source such as, for example, in the germline of a source organism of interest (e.g., of a human, a mouse, etc).

Reference: As used herein, the term “reference” describes a standard or control relative to which a comparison is performed. For example, in some aspects, an agent, animal, individual, population, sample, sequence or value of interest is compared with a reference or control agent, animal, individual, population, sample, sequence or value. In some aspects, a reference or control is tested and/or determined substantially simultaneously with the testing or determination of interest. In some aspects, a reference or control is a historical reference or control, optionally embodied in a tangible medium. Typically, as would be understood by those skilled in the art, a reference or control is determined or characterized under comparable conditions or circumstances to those under assessment. Those skilled in the art will appreciate when sufficient similarities are present to justify reliance on and/or comparison to a particular possible reference or control. In some aspects, a reference is a negative control reference; in some aspects, a reference is a positive control reference. In some aspects, the reference can be a compound, a protein, a polypeptide, or a polynucleotide disclosed in the present disclosure.

Regulatory Element: As used herein, the term “regulatory element” or “regulatory sequence” refers to non-coding regions of DNA that regulate, in some way, expression of one or more particular genes. In some aspects, such genes are apposed or “in the neighborhood” of a given regulatory element. In some aspects, such genes are located quite far from a given regulatory element. In some aspects, a regulatory element impairs or enhances transcription of one or more genes. In some aspects, a regulatory element may be located in cis to a gene being regulated. In some aspects, a regulatory element may be located in trans to a gene being regulated. For example, in some aspects, a regulatory sequence refers to a nucleic acid sequence which is regulates expression of a gene product operably linked to a regulatory sequence. In some such aspects, this sequence may be an enhancer sequence and other regulatory elements which regulate expression of a gene product.

Sample: As used herein, the term “sample” typically refers to an aliquot of material obtained or derived from a source of interest. In some aspects, a source of interest is a biological or environmental source. In some aspects, a source of interest may be or comprise a cell or an organism, such as a microbe (e.g., virus), a plant, or an animal (e.g., a human). In some aspects, a source of interest is or comprises biological tissue or fluid. In some aspects, a biological tissue or fluid may be or comprise amniotic fluid, aqueous humor, ascites, bile, bone marrow, blood, breast milk, cerebrospinal fluid, cerumen, chyle, chime, ejaculate, endolymph, exudate, feces, gastric acid, gastric juice, lymph, mucus, pericardial fluid, perilymph, peritoneal fluid, pleural fluid, pus, rheum, saliva, sebum, semen, serum, smegma, sputum, synovial fluid, sweat, tears, urine, vaginal secretions, vitreous humour, vomit, and/or combinations or component(s) thereof. In some aspects, a biological fluid may be or comprise an intracellular fluid, an extracellular fluid, an intravascular fluid (blood plasma), an interstitial fluid, a lymphatic fluid, and/or a transcellular fluid. In some aspects, a biological fluid may be or comprise a plant exudate. In some aspects, a biological tissue or sample may be obtained, for example, by aspirate, biopsy (e.g., fine needle or tissue biopsy), swab (e.g., oral, nasal, skin, or vaginal swab), scraping, surgery, washing or lavage (e.g., bronchioalveolar, ductal, nasal, ocular, oral, uterine, vaginal, or other washing or lavage). In some aspects, a biological sample is or comprises cells obtained from an individual. In some aspects, a sample is a “primary sample” obtained directly from a source of interest by any appropriate means. In some aspects, as will be clear from context, the term “sample” refers to a preparation that is obtained by processing (e.g., by removing one or more components of and/or by adding one or more agents to) a primary sample. For example, filtering using a semi-permeable membrane. Such a “processed sample” may comprise, for example nucleic acids or proteins extracted from a sample or obtained by subjecting a primary sample to one or more techniques such as amplification or reverse transcription of nucleic acid, isolation and/or purification of certain components, etc.

Selective expression: As used herein, the term “selective expression” or “selectively expresses” refers to expression of a gene or polypeptide of interest predominately in certain specific cell types (e.g., inner ear cells, e.g., inner ear supporting cells).

Subject: As used herein, the term “subject” refers to an organism, typically a mammal (e.g., a human, in some aspects including prenatal human forms). In some aspects, a subject is suffering from a relevant disease, disorder or condition. In some aspects, a subject is susceptible to a disease, disorder, or condition. In some aspects, a subject displays one or more symptoms or characteristics of a disease, disorder or condition. In some aspects, a subject does not display any symptom or characteristic of a disease, disorder, or condition. In some aspects, a subject is someone with one or more features characteristic of susceptibility to or risk of a disease, disorder, or condition. In some aspects, a subject is a patient. In some aspects, a subject is an individual to whom diagnosis and/or therapy is and/or has been administered.

Substantially: As used herein, the term “substantially” refers to a qualitative condition of exhibiting total or near-total extent or degree of a characteristic or property of interest. One of ordinary skill in the art will understand that biological and chemical phenomena rarely, if ever, go to completion and/or proceed to completeness or achieve or avoid an absolute result. The term “substantially” is therefore used herein to capture a potential lack of completeness inherent in many biological and chemical phenomena.

Supporting cell: As used herein, the term “support cell,” “supporting cell,” “inner ear support cell,” or “inner ear supporting cell” refers to cells of the inner ear that maintain the structure of the inner ear and maintain the environment of the sensory epithelium of the inner ear. In some aspects, inner ear supporting cells include, but are not limited to, inner phalangeal cells/border cells (IPhC), inner pillar cells (IPC), outer pillar cells (OPC), Deiters' cells rows 1 and 2 (DC1/2), Deiters' cells row 3 (DC3), Hensen's cells (Hec), Claudius cells/outer sulcus cells (CC/OSC), interdental cells (Idc), inner sulcus cells (ISC), Kölliker's organ cells (KO), greater ridge epithelial ridge cells (GER) (including lateral greater epithelial ridge cells (LGER)), and OC90+ cells (OC90), fibroblasts, and other cells of the lateral wall.

Supporting cell polypeptide: As used herein, the term “supporting cell polypeptide” or “support cell polypeptide” refers to a polypeptide that is endogenously expressed in a supporting cell of the inner ear.

Reporter polypeptide: As used herein, the term “reporter polypeptide” refers to a polypeptide that confers onto an organism or cell, a detectable or selectable phenotype. The detectable phenotype can be colorimetric, fluorescent or luminescent, for example. Reporter polypeptides can include enzymes mediating luminescence reactions (luxA, luxB, luxAB, luc, mc, nluc), enzymes mediating colorimetric reactions (lacZ, HRP), fluorescent proteins (GFP, eGFP, YFP, RFP, CFP, BFP, mCherry, near-infrared fluorescent proteins), affinity peptides (His-tag, 3X-FLAG), and selectable markers (ampC, tet(M), CAT, erm). The reporter polypeptide can be used as a marker for successful uptake of a nucleic acid molecule or exogenous sequence (plasmid) into a cell. The reporter polypeptide can also be used to indicate the presence of a target gene, target nucleic acid molecule, target polypeptide, target intracellular molecule, or a cell, as described herein.

Therapeutic polypeptide: As used herein, the term “therapeutic polypeptide” refers to a polypeptide possessing biological activity that can be used for the prevention and/or treatment of disease (e.g., hearing loss). Examples of therapeutic polypeptides include those capable of preventing, inhibiting, stabilizing or reversing an inherited or noninherited genetic defect in metabolism, immune regulation, hormonal regulation, enzymatic or membrane associated structural function. For example, therapeutic protein can replace an absent or defective cellular protein or enzyme, or supplement production of a defective or low expressed cellular protein or enzyme

Treatment: As used herein, the term “treatment” (also “treat” or “treating”) refers to any administration of a therapy that partially or completely alleviates, ameliorates, eliminates, reverses, relieves, inhibits, delays onset of, reduces severity of, and/or reduces incidence of one or more symptoms, features, and/or causes of a particular disease, disorder, and/or condition. In some aspects, such treatment may be of a subject who does not exhibit signs of the relevant disease, disorder and/or condition and/or of a subject who exhibits only early signs of the disease, disorder, and/or condition. Alternatively, or additionally, such treatment may be of a subject who exhibits one or more established signs of the relevant disease, disorder and/or condition. In some aspects, treatment may be of a subject who has been diagnosed as suffering from the relevant disease, disorder, and/or condition. In some aspects, treatment may be of a subject known to have one or more susceptibility factors that are statistically correlated with increased risk of development of a given disease, disorder, and/or condition.

Variant: As used herein, the term “variant” refers to a version of something, e.g., a gene sequence, that is different, in some way, from another version. To determine if something is a variant, a reference version is typically chosen and a variant is different relative to that reference version. In some aspects, a variant can have the same or a different (e.g., increased or decreased) level of activity or functionality than a wild type sequence. For example, in some aspects, a variant can have improved functionality as compared to a wild-type sequence if it is, e.g., codon-optimized to resist degradation, e.g., by an inhibitory nucleic acid, e.g., miRNA. Such a variant is referred to herein as a gain-of-function variant. In some aspects, a variant has a reduction or elimination in activity or functionality or a change in activity that results in a negative outcome (e.g., increased electrical activity resulting in chronic depolarization that leads to cell death). Such a variant is referred to herein as a loss-of-function variant. In some aspects, a gain-of-function variant is a codon-optimized sequence which encodes a transcript or polypeptide that may have improved properties (e.g., less susceptibility to degradation, e.g., less susceptibility to miRNA mediated degradation) than its corresponding wild type (e.g., non-codon optimized) version. In some aspects, a loss-of-function variant has one or more changes that result in a transcript or polypeptide that is defective in some way (e.g., decreased function, non-functioning) relative to the wild type transcript and/or polypeptide.

### DETAILED DESCRIPTION

In certain aspects, the present disclosure is directed to promoters for selective transgene expression, e.g., preferential expression in inner ear supporting cells.

In some aspects, the present disclosure is directed to constructs comprising a polynucleotide encoding a therapeutic polypeptide and compositions comprising the same which are designed for selective transgene expression, e.g., preferential expression in inner ear supporting cells and/or reduced expression in other inner ear cells such as hair cells.

In some aspects, the present disclosure is also directed to constructs comprising a polynucleotide encoding a polypeptide and compositions comprising the same which are designed for selective transgene expression, e.g., preferential expression in inner ear supporting cells and/or reduced expression in other inner ear cells such as hair cells.

In some aspects, the present disclosure is directed to constructs comprising a polynucleotide encoding a therapeutic polypeptide and compositions comprising the same which are designed for transgene expression in inner ear supporting cells, e.g., preferential expression in inner ear supporting cells and/or reduced expression in other inner ear cells such as hair cells. In some aspects, the preferential expression and/or reduced expression is relative to the corresponding endogenous expression.

In some aspects, the present disclosure is directed to AAV particles comprising the promoters or constructs disclosed herein.

In some aspects, the present disclosure is directed to methods of using the promoters, constructs, and AAV particles disclosed herein for treating hearing loss.

### Hearing Loss

Generally, an ear can be described as including: an outer ear, middle ear, inner ear, hearing (acoustic) nerve, and auditory system (which processes sound as it travels from the ear to the brain). In addition to detecting sound, ears also help to maintain balance. Thus, in some aspects, disorders of the inner ear can cause hearing loss, tinnitus, vertigo, imbalance, or combinations thereof.

Hearing loss can be the result of genetic factors, environmental factors, or a combination of genetic and environmental factors. About half of all people who have tinnitus—phantom noises in their auditory system (ringing, buzzing, chirping, humming, or beating)—also have an over-sensitivity to/reduced tolerance for certain sound frequency and volume ranges, known as hyperacusis (also spelled hyperacousis). A variety of nonsyndromic and syndromic-related hearing losses will be known to those of skill in the art (e.g., DFNB1 and DFNA3; and Bart-Pumphrey syndrome, hystrix-like ichthyosis with deafness (HID), palmoplantar keratoderma with deafness, keratitis-ichthyosis-deafness (KID) syndrome and Vohwinkel syndrome, respectively). Environmental causes of hearing impairment or loss may include, e.g., certain medications, specific infections before or after birth, and/or exposure to loud noise over an extended period. In some aspects, hearing loss can result from noise, ototoxic agents, presbycusis, disease, infection or cancers that affect specific parts of the ear. In some aspects, ischemic damage can cause hearing loss via pathophysiological mechanisms. In some aspects, intrinsic abnormalities, like congenital mutations to genes that play an important role in cochlear anatomy or physiology, or genetic or anatomical changes in supporting and/or hair cells can be responsible for or contribute to hearing loss.

Hearing loss and/or deafness is one of the most common human sensory deficits, and can occur for many reasons. In some aspects, a subject may be born with hearing loss or without hearing, while others may lose hearing slowly over time. Approximately 36 million American adults report some degree of hearing loss, and one in three people older than 60 and half of those older than 85 experience hearing loss. Approximately 1.5 in 1,000 children are born with profound hearing loss, and another two to three per 1,000 children are born with partial hearing loss (Smith et al., 2005, Lancet 365:879-890, which is incorporated in its entirety herein by reference). More than half of these cases are attributed to a genetic basis (Di Domenico, et al., 2011, J. Cell. Physiol. 226:2494-2499, which is incorporated in its entirety herein by reference).

Treatments for hearing loss currently consist of hearing amplification for mild to severe losses and cochlear implantation for severe to profound losses (Kral and O'Donoghue, 2010, N. Engl. J. Med. 363:1438-1450, which is incorporated in its entirety herein by reference). Recent research in this arena has focused on cochlear hair cell regeneration, applicable to the most common forms of hearing loss, including presbycusis, noise damage, infection, and ototoxicity. There remains a need for effective treatments, such as gene therapy, which can repair and/or mitigate a source of a hearing problem (see e.g., WO 2018/039375, WO 2019/165292, and PCT filing application US2019/060328, each of which is incorporated in its entirety herein by reference).

In some aspects, nonsyndromic hearing loss and/or deafness is not associated with other signs and symptoms. In some aspects, syndromic hearing loss and/or deafness occurs in conjunction with abnormalities in other parts of the body. Approximately 70 percent to 80 percent of genetic hearing loss and/or deafness cases are nonsyndromic; remaining cases are often caused by specific genetic syndromes. Nonsyndromic deafness and/or hearing loss can have different patterns of inheritance, and can occur at any age. Types of nonsyndromic deafness and/or hearing loss are generally named according to their inheritance patterns. For example, autosomal dominant forms are designated DFNA, autosomal recessive forms are DFNB, and X-linked forms are DFN. Each type is also numbered in the order in which it was first described. For example, DFNA1 was the first described autosomal dominant type of nonsyndromic deafness. Between 75 percent and 80 percent of genetically causative hearing loss and/or deafness cases are inherited in an autosomal recessive pattern, which means both copies of the gene in each cell have mutations. Usually, each parent of an individual with autosomal recessive hearing loss and/or deafness is a carrier of one copy of the mutated gene, but is not affected by this form of hearing loss. Another 20 percent to 25 percent of nonsyndromic hearing loss and/or deafness cases are autosomal dominant, which means one copy of the altered gene in each cell is sufficient to result in deafness and/or hearing loss. People with autosomal dominant deafness and/or hearing loss most often inherit an altered copy of the gene from a parent who is deaf and/or has hearing loss. Between 1 to 2 percent of cases of deafness and/or hearing loss show an X-linked pattern of inheritance, which means the mutated gene responsible for the condition is located on the X chromosome (one of the two sex chromosomes). Males with X-linked nonsyndromic hearing loss and/or deafness tend to develop more severe hearing loss earlier in life than females who inherit a copy of the same gene mutation. A characteristic of X-linked inheritance is that fathers cannot pass X-linked traits to their sons. Mitochondrial nonsyndromic deafness, which results from changes to mitochondrial DNA, occurs in less than one percent of cases in the United States. The altered mitochondrial DNA is passed from a mother to all of her sons and daughters. This type of deafness is not inherited from fathers. The causes of syndromic and nonsyndromic deafness and/or hearing loss are complex. Researchers have identified more than 30 genes that, when altered, are associated with syndromic and/or nonsyndromic deafness and/or hearing loss; however, some of these genes have not been fully characterized. Different mutations in the same gene can be associated with different types of deafness and/or hearing loss, and some genes are associated with both syndromic and nonsyndromic deafness and/or hearing loss.

In some aspects, deafness and/or hearing loss can be conductive (arising from the ear canal or middle ear), sensorineural (arising from the inner ear or auditory nerve), or mixed. In some aspects, nonsyndromic deafness and/or hearing loss is associated with permanent hearing loss caused by damage to structures in the inner ear (sensorineural deafness). In some aspects, sensorineural hearing loss can be due to poor hair cell function. In some aspects, sensorineural hearing impairments involve the eighth cranial nerve (the vestibulocochlear nerve) or the auditory portions of the brain. In some such aspects, only the auditory centers of the brain are affected. In such a situation, cortical deafness may occur, where sounds may be heard at normal thresholds, but quality of sound perceived is so poor that speech cannot be understood. Hearing loss that results from changes in the middle ear is called conductive hearing loss. Some forms of nonsyndromic deafness and/or hearing loss involve changes in both the inner ear and the middle ear, called mixed hearing loss. Hearing loss and/or deafness that is present before a child learns to speak can be classified as prelingual or congenital. Hearing loss and/or deafness that occurs after the development of speech can be classified as postlingual. Most autosomal recessive loci related to syndromic or nonsyndromic hearing loss cause prelingual severe-to-profound hearing loss.

As is known to those of skill in the art, hair cells are sensory receptors for both auditory and vestibular systems of vertebrate ears. Hair cells detect movement in the environment and, in mammals, hair cells are located within the cochlea of the ear, in the organ of Corti. Mammalian ears are known to have two types of hair cells—inner hair cells and outer hair cells. Outer hair cells can amplify low level sound frequencies, either through mechanical movement of hair cell bundles or electrically-driven movement of hair cell soma. Inner hair cells transform vibrations in cochlear fluid into electrical signals that the auditory nerve transmits to the brain. In some aspects, hair cells may be abnormal at birth, or damaged during the lifetime of an individual. In some aspects, outer hair cells may be able to regenerate. In some aspects, inner hair cells are not capable of regeneration after illness or injury. In some aspects, sensorineural hearing loss is due to abnormalities in hair cells.

As is known to those of skill in the art, hair cells do not occur in isolation, and their function is supported by a wide variety of cells which can collectively be referred to as supporting cells. Supporting cells may fulfill numerous functions, and include a number of cell types, including but not limited to inner phalangeal cells/border cells (IPhC), inner pillar cells (IPC), outer pillar cells (OPC), Deiters' cells rows 1 and 2 (DC1/2), Deiters' cells row 3 (DC3), Hensen's cells (Hec), Claudius cells/outer sulcus cells (CC/OSC), interdental cells (Idc), inner sulcus cells (ISC), Kölliker's organ cells (KO), greater ridge epithelial ridge cells (GER) (including lateral greater epithelial ridge cells (LGER)), and OC90+ cells (OC90), fibroblasts, and other cells of the lateral wall.

In some aspects, sensorineural hearing loss is due to abnormalities in supporting cells. In some aspects, supporting cells may be abnormal at birth, or damaged during the lifetime of an individual. In some aspects, supporting cells may be able to regenerate. In some aspects, certain supporting cells may not be capable of regeneration.

### Polypeptides

Certain aspects of the disclosure are directed to polynucleotides encoding a polypeptide (e.g., a Connexin 26 polypeptide). The polynucleotide can encode a polypeptide that is capable of being expressed in a cell (e.g., an inner ear cell). The polynucleotide can encode a full length polypeptide or a functional fragment thereof.

Exemplary polypeptides encoded by the polynucleotide include, but are not limited to, transmembrane proteins, enzymes, growth factors, cytokines, receptors, receptor ligands, hormones, membrane proteins, membrane-associated proteins, antigens, and antibodies.

Exemplary polynucleotides encoding polypeptides include, but are not limited to, ATPase Plasma Membrane Ca2+ Transporting 2 (ATP2B2), Cholinergic Receptor Nicotinic Alpha 9 Subunit (CHRNA9), Cadherin 23 (CDH23), Coiled-coil Glutamate Rich Protein 2 (CCER2), Clarin 1 (CLRN1), Clarin 2 (CLRN2), cochlin (COCH or DFNA9), Dystrotelin (DYTN), Epidermal Growth Factor Receptor Pathway Substrate 8 (EPS8), EPS8 Like 2 (EPS8L2), Espin (ESPN), Espin Like (ESPNL), Gap junction protein beta 2 (GJB2), Gap junction protein beta 6 (GJB6), Gap junction protein beta 3(GJB3), gasdermin E protein (GSDME or DFNA5), Insulinoma-associated 1 (INSM1), Ikaros family zinc finger 2 (IKZF2), LIM Homeobox Protein 3 (LHX3), Myosin 7A (MYO7A), Myosin 11 (MYO3A), Norrin cystine knot growth factor (NDP), Protocadherin 15 (PCDH15), Protein Tyrosine Phosphatase, Receptor Type Q (PTPRQ), Stereocilin (STRC), Protein Network Component Harmonin (USH1C), Usherin (USH2A), and Spectrin repeat containing nuclear envelope family member 4 (SYNE4).

In some aspects, the polypeptide is a therapeutic polypeptide. In some aspects, the polypeptide is a supporting cell polypeptide. In some aspects, the polypeptide is a reporter polypeptide.

### Supporting Cell Polypeptides

Certain aspects of the disclosure are directed to polynucleotides encoding a supporting cell polypeptide. The polynucleotide can encode a polypeptide that is capable of being expressed in a cell (e.g., an inner ear cell). In some aspects, the supporting cell polypeptide is a poypeptide that is endogenously expressed in supporting cells of the inner ear. In some aspects, the inner ear supporting cells are selected from one or more of inner phalangeal cells/border cells (IPhC), inner pillar cells (IPC), outer pillar cells (OPC), Deiters' cells rows 1 and 2 (DC1/2), Deiters' cells row 3 (DC3), Hensen's cells (Hec), Claudius cells/outer sulcus cells (CC/OSC), interdental cells (Idc), inner sulcus cells (ISC), Kölliker's organ cells (KO), greater ridge epithelial ridge cells (GER) (including lateral greater epithelial ridge cells (LGER)), and OC90+ cells (OC90), fibroblasts, and other cells of the lateral wall. The polynucleotide can encode a full length polypeptide or a functional fragment thereof.

Exemplary supporting cell polypeptides encoded by the polynucleotide include, but are not limited to, transmembrane proteins, enzymes, growth factors, cytokines, receptors, receptor ligands, hormones, membrane proteins, membrane-associated proteins, antigens, and antibodies.

Exemplary supporting cell polynucleotides encoding polypeptides include, but are not limited to, ATPase Plasma Membrane Ca2+ Transporting 2 (ATP2B2), Cholinergic Receptor Nicotinic Alpha 9 Subunit (CHRNA9), Cadherin 23 (CDH23), Coiled-coil Glutamate Rich Protein 2 (CCER2), Clarin 1 (CLRN1), Clarin 2 (CLRN2), cochlin (COCH or DFNA9), Dystrotelin (DYTN), Epidermal Growth Factor Receptor Pathway Substrate 8 (EPS8), EPS8 Like 2 (EPS8L2), Espin (ESPN), Espin Like (ESPNL), Gap junction protein beta 2 (GJB2), Gap junction protein beta 6 (GJB6), Gap junction protein beta 3(GJB3), gasdermin E protein (GSDME or DFNA5), Insulinoma-associated 1 (INSM1), Ikaros family zinc finger 2 (IKZF2), LIM Homeobox Protein 3 (LHX3), Myosin 7A (MYO7A), Myosin 11 (MYO3A), Norrin cystine knot growth factor (NDP), Protocadherin 15 (PCDH15), Protein Tyrosine Phosphatase, Receptor Type Q (PTPRQ), Stereocilin (STRC), Protein Network Component Harmonin (USH1C), Usherin (USH2A), and Spectrin repeat containing nuclear envelope family member 4 (SYNE4).

### Therapeutic Polypeptides

Certain aspects of the disclosure are directed to polynucleotides encoding a polypeptide (e.g., a therapeutic polypeptide). The polynucleotide can encode a polypeptide that is capable of being expressed in a cell (e.g., an inner ear cell). The polynucleotide can encode a full length polypeptide or a functional fragment thereof.

Exemplary polypeptides encoded by the polynucleotide include, but are not limited to, transmembrane proteins, enzymes, growth factors, cytokines, receptors, receptor ligands, hormones, membrane proteins, membrane-associated proteins, antigens, and antibodies.

Exemplary polynucleotides encoding therapeutic polypeptides include, but are not limited to, ATPase Plasma Membrane Ca2+ Transporting 2 (ATP2B2), Cholinergic Receptor Nicotinic Alpha 9 Subunit (CHRNA9), Cadherin 23 (CDH23), Coiled-coil Glutamate Rich Protein 2 (CCER2), Clarin 1 (CLRN1), Clarin 2 (CLRN2), cochlin (COCH or DFNA9), Dystrotelin (DYTN), Epidermal Growth Factor Receptor Pathway Substrate 8 (EPS8), EPS8 Like 2 (EPS8L2), Espin (ESPN), Espin Like (ESPNL), Gap junction protein beta 2 (GJB2), Gap junction protein beta 6 (GJB6), Gap junction protein beta 3(GJB3), gasdermin E protein (GSDME or DFNA5), Insulinoma-associated 1 (INSM1), Ikaros family zinc finger 2 (IKZF2), LIM Homeobox Protein 3 (LHX3), Myosin 7A (MYO7A), Myosin 11 (MYO3A), Norrin cystine knot growth factor (NDP), Protocadherin 15 (PCDH15), Protein Tyrosine Phosphatase, Receptor Type Q (PTPRQ), Stereocilin (STRC), Protein Network Component Harmonin (USH1C), Usherin (USH2A), and Spectrin repeat containing nuclear envelope family member 4 (SYNE4).

### Constructs

Among other things, the present disclosure provides that some polynucleotides as described herein are polynucleotide constructs. Polynucleotide constructs according to the present disclosure include all those known in the art, including cosmids, plasmids (e.g., naked or contained in liposomes) and viral constructs (e.g., lentiviral, retroviral, adenoviral, and adeno-associated viral constructs) that incorporate a polynucleotide comprising a nucleic acid sequence (e.g., GJB2 gene) or characteristic portion thereof encoding a polypeptide (e.g., Connexin 26). Those of skill in the art will be capable of selecting suitable constructs, as well as cells, for making any of the polynucleotides described herein. In some aspects, a construct is a plasmid (i.e., a circular DNA molecule that can autonomously replicate inside a cell). In some aspects, a construct can be a cosmid (e.g., pWE or sCos series). In some aspects, the construct is a mammalian or a viral vector.

In some aspects, a construct is a viral construct. In some aspects, a viral construct is a lentivirus, retrovirus, adenovirus, or adeno-associated virus construct. In some aspects, a construct is an adeno-associated virus (AAV) construct (see, e.g., Asokan et al., Mol. Ther. 20: 699-7080, 2012, which is incorporated in its entirety herein by reference). In some aspects, the construct is a viral vector. In some aspects, the construct is a lentivirus, retrovirus, adenovirus, or adeno-associated virus vector. In some aspects, the construct is an AAV vector. In some aspects, a viral construct is an adenovirus construct. In some aspects, a viral construct may also be based on or derived from an alphavirus. Alphaviruses include Sindbis (and VEEV) virus, Aura virus, Babanki virus, Barmah Forest virus, Bebaru virus, Cabassou virus, Chikungunya virus, Eastern equine encephalitis virus, Everglades virus, Fort Morgan virus, Getah virus, Highlands J virus, Kyzylagach virus, Mayaro virus, Me Tri virus, Middelburg virus, Mosso das Pedras virus, Mucambo virus, Ndumu virus, O'nyong-nyong virus, Pixuna virus, Rio Negro virus, Ross River virus, Salmon pancreas disease virus, Semliki Forest virus, Southern elephant seal virus, Tonate virus, Trocara virus, Una virus, Venezuelan equine encephalitis virus, Western equine encephalitis virus, and Whataroa virus. Generally, the genome of such viruses encode nonstructural (e.g., replicon) and structural proteins (e.g., capsid and envelope) that can be translated in the cytoplasm of the host cell. Ross River virus, Sindbis virus, Semliki Forest virus (SFV), and Venezuelan equine encephalitis virus (VEEV) have all been used to develop viral constructs for coding sequence delivery. Pseudotyped viruses may be formed by combining alphaviral envelope glycoproteins and retroviral capsids. Examples of alphaviral constructs can be found in U.S. Publication Nos. 20150050243, 20090305344, and 20060177819; constructs and methods of their making are incorporated herein by reference to each of the publications in its entirety.

Constructs provided herein can be of different sizes. In some aspects, a construct is a plasmid and can include a total length of up to about 1 kb, up to about 2 kb, up to about 3 kb, up to about 4 kb, up to about 5 kb, up to about 6 kb, up to about 7 kb, up to about 8 kb, up to about 9 kb, up to about 10 kb, up to about 11 kb, up to about 12 kb, up to about 13 kb, up to about 14 kb, or up to about 15 kb. In some aspects, a construct is a plasmid and can have a total length in a range of about 1 kb to about 2 kb, about 1 kb to about 3 kb, about 1 kb to about 4 kb, about 1 kb to about 5 kb, about 1 kb to about 6 kb, about 1 kb to about 7 kb, about 1 kb to about 8 kb, about 1 kb to about 9 kb, about 1 kb to about 10 kb, about 1 kb to about 11 kb, about 1 kb to about 12 kb, about 1 kb to about 13 kb, about 1 kb to about 14 kb, or about 1 kb to about 15 kb.

In some aspects, a construct is a viral construct and can have a total number of nucleotides of up to 10 kb. In some aspects, a viral construct can have a total number of nucleotides in the range of about 1 kb to about 2 kb, 1 kb to about 3 kb, about 1 kb to about 4 kb, about 1 kb to about 5 kb, about 1 kb to about 6 kb, about 1 kb to about 7 kb, about 1 kb to about 8 kb, about 1 kb to about 9 kb, about 1 kb to about 10 kb, about 2 kb to about 3 kb, about 2 kb to about 4 kb, about 2 kb to about 5 kb, about 2 kb to about 6 kb, about 2 kb to about 7 kb, about 2 kb to about 8 kb, about 2 kb to about 9 kb, about 2 kb to about 10 kb, about 3 kb to about 4 kb, about 3 kb to about 5 kb, about 3 kb to about 6 kb, about 3 kb to about 7 kb, about 3 kb to about 8 kb, about 3 kb to about 9 kb, about 3 kb to about 10 kb, about 4 kb to about 5 kb, about 4 kb to about 6 kb, about 4 kb to about 7 kb, about 4 kb to about 8 kb, about 4 kb to about 9 kb, about 4 kb to about 10 kb, about 5 kb to about 6 kb, about 5 kb to about 7 kb, about 5 kb to about 8 kb, about 5 kb to about 9 kb, about 5 kb to about 10 kb, about 6 kb to about 7 kb, about 6 kb to about 8 kb, about 6 kb to about 9 kb, about 6 kb to about 10 kb, about 7 kb to about 8 kb, about 7 kb to about 9 kb, about 7 kb to about 10 kb, about 8 kb to about 9 kb, about 8 kb to about 10 kb, or about 9 kb to about 10 kb.

In some aspects, a construct is a lentivirus construct and can have a total number of nucleotides of up to 8 kb. In some examples, a lentivirus construct can have a total number of nucleotides of about 1 kb to about 2 kb, about 1 kb to about 3 kb, about 1 kb to about 4 kb, about 1 kb to about 5 kb, about 1 kb to about 6 kb, about 1 kb to about 7 kb, about 1 kb to about 8 kb, about 2 kb to about 3 kb, about 2 kb to about 4 kb, about 2 kb to about 5 kb, about 2 kb to about 6 kb, about 2 kb to about 7 kb, about 2 kb to about 8 kb, about 3 kb to about 4 kb, about 3 kb to about 5 kb, about 3 kb to about 6 kb, about 3 kb to about 7 kb, about 3 kb to about 8 kb, about 4 kb to about 5 kb, about 4 kb to about 6 kb, about 4 kb to about 7 kb, about 4 kb to about 8 kb, about 5 kb to about 6 kb, about 5 kb to about 7 kb, about 5 kb to about 8 kb, about 6 kb to about 8 kb, about 6 kb to about 7 kb, or about 7 kb to about 8 kb.

In some aspects, a construct is an adeno-associated virus construct and can have a total number of nucleotides of up to 8 kb. In some aspects, an adeno-associated virus construct can have a total number of nucleotides in the range of about 1 kb to about 2 kb, about 1 kb to about 3 kb, about 1 kb to about 4 kb, about 1 kb to about 5 kb, about 1 kb to about 6 kb, about 1 kb to about 7 kb, about 1 kb to about 8 kb, about 2 kb to about 3 kb, about 2 kb to about 4 kb, about 2 kb to about 5 kb, about 2 kb to about 6 kb, about 2 kb to about 7 kb, about 2 kb to about 8 kb, about 3 kb to about 4 kb, about 3 kb to about 5 kb, about 3 kb to about 6 kb, about 3 kb to about 7 kb, about 3 kb to about 8 kb, about 4 kb to about 5 kb, about 4 kb to about 6 kb, about 4 kb to about 7 kb, about 4 kb to about 8 kb, about 5 kb to about 6 kb, about 5 kb to about 7 kb, about 5 kb to about 8 kb, about 6 kb to about 7 kb, about 6 kb to about 8 kb, or about 7 kb to about 8 kb.

In some aspects, a construct is an adenovirus construct and can have a total number of nucleotides of up to 8 kb. In some aspects, an adenovirus construct can have a total number of nucleotides in the range of about 1 kb to about 2 kb, about 1 kb to about 3 kb, about 1 kb to about 4 kb, about 1 kb to about 5 kb, about 1 kb to about 6 kb, about 1 kb to about 7 kb, about 1 kb to about 8 kb, about 2 kb to about 3 kb, about 2 kb to about 4 kb, about 2 kb to about 5 kb, about 2 kb to about 6 kb, about 2 kb to about 7 kb, about 2 kb to about 8 kb, about 3 kb to about 4 kb, about 3 kb to about 5 kb, about 3 kb to about 6 kb, about 3 kb to about 7 kb, about 3 kb to about 8 kb, about 4 kb to about 5 kb, about 4 kb to about 6 kb, about 4 kb to about 7 kb, about 4 kb to about 8 kb, about 5 kb to about 6 kb, about 5 kb to about 7 kb, about 5 kb to about 8 kb, about 6 kb to about 7 kb, about 6 kb to about 8 kb, or about 7 kb to about 8 kb.

Any of the constructs described herein can further include a control sequence, e.g., a control sequence selected from the group of a transcription initiation sequence, a transcription termination sequence, a promoter sequence, an enhancer sequence, an RNA splicing sequence, a polyadenylation (poly(A)) sequence, a Kozak consensus sequence, and/or additional untranslated regions which may house pre- or post-transcriptional regulatory and/or control elements. In some aspects, a promoter can be a native promoter, a constitutive promoter, an inducible promoter, and/or a tissue-specific promoter. Non-limiting examples of control sequences are described herein.

In some aspects, the construct comprises a polynucleotide encoding a therapeutic polypeptide operably linked to a promoter which selectively expresses the polynucleotide in an inner ear support cell. In some aspects, the construct comprise a 5′ ITR, a promoter which selectively expresses the polynucleotide in an inner ear support cell, a 5′ UTR, a polynucleotide encoding a therapeutic polypeptide, a 3′ UTR, a polyA, and a 3′ ITR. In some aspects, the construct comprise a 5′ ITR, a promoter which selectively expresses the polynucleotide in an inner ear support cell, a 5′ UTR, a polynucleotide encoding a therapeutic polypeptide, a tag, a 3′ UTR, a polyA, and a 3′ ITR. In some aspects, the construct comprise a 5′ ITR, a promoter which selectively expresses the polynucleotide in an inner ear support cell, a 5′ UTR, a polynucleotide encoding a therapeutic polypeptide, a tag, a 3′ UTR, a microRNA regulatory target site, a polyA, and a 3′ ITR.

In some aspects, the construct comprises a polynucleotide encoding a polypeptide operably linked to a promoter which selectively expresses the polynucleotide in an inner ear support cell. In some aspects, the construct comprise a 5′ ITR, a promoter which selectively expresses the polynucleotide in an inner ear support cell, a 5′ UTR, a polynucleotide encoding a polypeptide, a 3′ UTR, a polyA, and a 3′ ITR. In some aspects, the construct comprise a 5′ ITR, a promoter which selectively expresses the polynucleotide in an inner ear support cell, a 5′ UTR, a polynucleotide encoding a polypeptide, a tag, a 3′ UTR, a polyA, and a 3′ ITR. In some aspects, the construct comprise a 5′ ITR, a promoter which selectively expresses the polynucleotide in an inner ear support cell, a 5′ UTR, a polynucleotide encoding a polypeptide, a tag, a 3′ UTR, a microRNA regulatory target site, a polyA, and a 3′ ITR.

In some aspects, the construct comprises a polynucleotide encoding a therapeutic polypeptide operably linked to a promoter, wherein the construct comprises a miRNA regulatory target site for a microRNA expressed in an inner ear cell. In some aspects, the construct comprises a 5′ ITR, a promoter which selectively expresses the polynucleotide in an inner ear support cell, a 5′ UTR, a polynucleotide encoding a therapeutic polypeptide, a 3′ UTR, a microRNA regulatory target site, a polyA, and a 3′ ITR. In some aspects, the construct comprises a 5′ ITR, a promoter which selectively expresses the polynucleotide in an inner ear support cell, a 5′ UTR, a polynucleotide encoding a therapeutic polypeptide, a tag, a 3′ UTR, a microRNA regulatory target site, a polyA, and a 3′ ITR. In some aspects, the construct comprises a 5′ ITR, a constitutive promoter, a 5′ UTR, a polynucleotide encoding a therapeutic polypeptide, a 3′ UTR, a microRNA regulatory target site, a polyA, and a 3′ ITR. In some aspects, the construct comprises a 5′ ITR, a constitutive promoter, a 5′ UTR, a polynucleotide encoding a therapeutic polypeptide, a tag, a 3′ UTR, a microRNA regulatory target site, a polyA, and a 3′ ITR.

In some aspects, the construct comprises a polynucleotide encoding a polypeptide operably linked to a promoter, wherein the construct comprises a miRNA regulatory target site for a microRNA expressed in an inner ear cell (e.g., a hair cell). In some aspects, the construct comprises a 5′ ITR, a promoter which selectively expresses the polynucleotide in an inner ear support cell, a 5′ UTR, a polynucleotide encoding a polypeptide, a 3′ UTR, a microRNA regulatory target site, a polyA, and a 3′ ITR. In some aspects, the construct comprises a 5′ ITR, a promoter which selectively expresses the polynucleotide in an inner ear support cell, a 5′ UTR, a polynucleotide encoding a polypeptide, a tag, a 3′ UTR, a microRNA regulatory target site, a polyA, and a 3′ ITR. In some aspects, the construct comprises a 5′ ITR, a constitutive promoter, a 5′ UTR, a polynucleotide encoding a polypeptide, a 3′ UTR, a microRNA regulatory target site, a polyA, and a 3′ ITR. In some aspects, the construct comprises a 5′ ITR, a constitutive promoter, a 5′ UTR, a polynucleotide encoding a polypeptide, a tag, a 3′ UTR, a microRNA regulatory target site, a polyA, and a 3′ ITR.

In some aspects, the construct comprises (i) a 5′ inverted terminal repeat (ITR), (ii) a polynucleotide encoding a polypeptide operably linked to a promoter which expresses the polynucleotide in an inner ear support cell, and (iii) a 3′ ITR, wherein the promoter is heterologous to the polynucleotide.

In some aspects, the construct comprises (i) a 5′ inverted terminal repeat (ITR), (ii) a polynucleotide encoding a polypeptide operably linked to a promoter, and (iii) a 3′ ITR, wherein the promoter comprises a nucleic acid sequence having at least 85%, at least 90%, at least 95%, at least 96%, at least 97%, at least 98%, at least 99%, or 100% identity to any one of SEQ ID NOs: 16, 28, 40, 57, or 90-99. In some aspects, the construct further comprises a minimal GJB2 promoter. In some aspects, the minimal GJB2 promoter comprises a nucleic acid sequence at least 80%, at least 85%, at least 90%, at least 95%, at least 96%, at least 97%, at least 98%, at least 99%, or 100% identity to SEQ ID NO: 86.

In some aspects, the construct comprises (i) the 5′ inverted terminal repeat (ITR), (ii) a 5′ untranslated region (UTR), (iii) the polynucleotide encoding a polypeptide operably linked to a promoter which expresses the polynucleotide in an inner ear support cell, and (iv) the 3′ ITR.

In some aspects, the construct comprises (i) the 5′ inverted terminal repeat (ITR), (ii) a 5′ untranslated region (UTR), (iii) the polynucleotide encoding a polypeptide operably linked to a promoter, and (iv) the 3′ ITR.

In some aspects, the construct comprises (i) the 5′ inverted terminal repeat (ITR), (ii) the polynucleotide encoding a polypeptide operably linked to a promoter which expresses the polynucleotide in an inner ear support cell, (iii) a 3′ untranslated region (UTR), and (iv) the 3′ ITR.

In some aspects, the construct comprises (i) the 5′ inverted terminal repeat (ITR), (ii) the polynucleotide encoding a polypeptide operably linked to a promoter, (iii) a 3′ untranslated region (UTR), and (iv) the 3′ ITR.

In some aspects, the construct comprises (i) the 5′ inverted terminal repeat (ITR), (ii) the polynucleotide encoding a polypeptide operably linked to a promoter which expresses the polynucleotide in an inner ear support cell, (iii) a miRNA regulatory target site (miRTS) for a microRNA expressed in an inner ear cell, (iv) a 3′ untranslated region (UTR), and (v) the 3′ ITR.

In some aspects, the construct comprises (i) the 5′ inverted terminal repeat (ITR), (ii) the polynucleotide encoding a polypeptide operably linked to a promoter, (iii) a miRNA regulatory target site (miRTS) for a microRNA expressed in an inner ear cell, (iv) a 3′ untranslated region (UTR), and (v) the 3′ ITR.

In some aspects, the construct comprises (i) the 5′ inverted terminal repeat (ITR), (ii) a 5′ untranslated region (UTR), (iii) the polynucleotide encoding a polypeptide operably linked to a promoter which expresses the polynucleotide in an inner ear support cell, (iv) a 3′ UTR, and (v) the 3′ ITR.

In some aspects, the construct comprises (i) the 5′ inverted terminal repeat (ITR), (ii) a 5′ untranslated region (UTR), (iii) the polynucleotide encoding a polypeptide operably linked to a promoter, (iv) a 3′ UTR, and (v) the 3′ ITR.

In some aspects, the construct comprises (i) the 5′ inverted terminal repeat (ITR), (ii) a 5′ untranslated region (UTR), (iii) the polynucleotide encoding a polypeptide operably linked to a promoter which expresses the polynucleotide in an inner ear support cell, (iii) a miRNA regulatory target site (miRTS) for a microRNA expressed in an inner ear cell, (v) a 3′ UTR, and (vi) the 3′ ITR.

In some aspects, the construct comprises (i) the 5′ inverted terminal repeat (ITR), (ii) a 5′ untranslated region (UTR), (iii) the polynucleotide encoding a polypeptide operably linked to a promoter, (iv) a miRNA regulatory target site (miRTS) for a microRNA expressed in an inner ear cell, (v) a 3′ UTR, and (vi) the 3′ ITR.

In some aspects, the construct comprises a polynucleotide encoding a polypeptide operably linked to a promoter, wherein the construct comprises a miRNA regulatory target site (miRTS) for a microRNA expressed in an inner ear cell.

In some aspects, the construct comprises (i) a 5′ inverted terminal repeat (ITR), (ii) the polynucleotide encoding a polypeptide operably linked to a promoter, (iii) the miRNA regulatory target site (miRTS) for a microRNA expressed in an inner ear cell, and (iv) a 3′ ITR.

In some aspects, the construct comprises (i) a 5′ inverted terminal repeat (ITR), (ii) a 5′ untranslated region (UTR), (iii) the polynucleotide encoding a polypeptide operably linked to a promoter, (iv) the miRNA regulatory target site (miRTS) for a microRNA expressed in an inner ear cell, and (v) a 3′ ITR.

In some aspects, the construct comprises (i) a 5′ inverted terminal repeat (ITR), (ii) the polynucleotide encoding a polypeptide operably linked to a promoter, (iii) the miRNA regulatory target site (miRTS) for a microRNA expressed in an inner ear cell, (iv) a 3′ untranslated region (UTR) and (v) a 3′ ITR.

In some aspects, the construct comprises (i) a 5′ inverted terminal repeat (ITR), (ii) a 5′ untranslated region (UTR), (iii) the polynucleotide encoding a polypeptide operably linked to a promoter, (iv) the miRNA regulatory target site (miRTS) for a microRNA expressed in an inner ear cell, (v) a 3′ UTR, and (vi) a 3′ ITR.

In some aspects, the construct comprises (i) a 5′ inverted terminal repeat (ITR), (ii) a polynucleotide encoding a polypeptide operably linked to an inner ear supporting cell selective promoter and a minimal GJB2 promoter which expresses the polynucleotide in an inner ear support cell, and (iii) a 3′ ITR, wherein the inner ear supporting cell selective promoter is heterologous to the polynucleotide.

In some aspects, the construct comprises (i) a 5′ inverted terminal repeat (ITR), (ii) a polynucleotide encoding a polypeptide operably linked to an inner ear supporting cell selective promoter and a minimal GJB2 promoter, and (iii) a 3′ ITR, wherein the inner ear supporting cell selective promoter comprises a nucleic acid sequence having at least 85%, at least 90%, at least 95%, at least 96%, at least 97%, at least 98%, at least 99%, or 100% identity to any one of SEQ ID NOs: 16, 28, 40, 57, or 90-99.

In some aspects, the construct comprises (i) the 5′ inverted terminal repeat (ITR), (ii) a 5′ untranslated region (UTR), (iii) the polynucleotide encoding a polypeptide operably linked to an inner ear supporting cell selective promoter and a minimal GJB2 promoter which expresses the polynucleotide in an inner ear support cell, and (iv) the 3′ ITR.

In some aspects, the construct comprises (i) the 5′ inverted terminal repeat (ITR), (ii) a 5′ untranslated region (UTR), (iii) the polynucleotide encoding a polypeptide operably linked to an inner ear supporting cell selective promoter and a minimal GJB2 promoter, and (iv) the 3′ ITR.

In some aspects, the construct comprises (i) the 5′ inverted terminal repeat (ITR), (ii) the polynucleotide encoding a polypeptide operably linked to an inner ear supporting cell selective promoter and a minimal GJB2 promoter which expresses the polynucleotide in an inner ear support cell, (iii) a 3′ untranslated region (UTR), and (iv) the 3′ ITR.

In some aspects, the construct comprises (i) the 5′ inverted terminal repeat (ITR), (ii) the polynucleotide encoding a polypeptide operably linked to an inner ear supporting cell selective promoter and a minimal GJB2 promoter, (iii) a 3′ untranslated region (UTR), and (iv) the 3′ ITR.

In some aspects, the construct comprises (i) the 5′ inverted terminal repeat (ITR), (ii) the polynucleotide encoding a polypeptide operably linked to an inner ear supporting cell selective promoter and a minimal GJB2 promoter which expresses the polynucleotide in an inner ear support cell, (iii) a miRNA regulatory target site (miRTS) for a microRNA expressed in an inner ear cell, (iv) a 3′ untranslated region (UTR), and (v) the 3′ ITR.

In some aspects, the construct comprises (i) the 5′ inverted terminal repeat (ITR), (ii) the polynucleotide encoding a polypeptide operably linked to an inner ear supporting cell selective promoter and a minimal GJB2 promoter, (iii) a miRNA regulatory target site (miRTS) for a microRNA expressed in an inner ear cell, (iv) a 3′ untranslated region (UTR), and (v) the 3′ ITR.

In some aspects, the construct comprises (i) the 5′ inverted terminal repeat (ITR), (ii) a 5′ untranslated region (UTR), (iii) the polynucleotide encoding a polypeptide operably linked to an inner ear supporting cell selective promoter and a minimal GJB2 promoter which expresses the polynucleotide in an inner ear support cell, (iv) a 3′ UTR, and (v) the 3′ ITR.

In some aspects, the construct comprises (i) the 5′ inverted terminal repeat (ITR), (ii) a 5′ untranslated region (UTR), (iii) the polynucleotide encoding a polypeptide operably linked to an inner ear supporting cell selective promoter and a minimal GJB2 promoter, (iv) a 3′ UTR, and (v) the 3′ ITR.

In some aspects, the construct comprises (i) the 5′ inverted terminal repeat (ITR), (ii) a 5′ untranslated region (UTR), (iii) the polynucleotide encoding a polypeptide operably linked to an inner ear supporting cell selective promoter and a minimal GJB2 promoter which expresses the polynucleotide in an inner ear support cell, (iii) a miRNA regulatory target site (miRTS) for a microRNA expressed in an inner ear cell, (v) a 3′ UTR, and (vi) the 3′ ITR.

In some aspects, the construct comprises (i) the 5′ inverted terminal repeat (ITR), (ii) a 5′ untranslated region (UTR), (iii) the polynucleotide encoding a polypeptide operably linked to an inner ear supporting cell selective promoter and a minimal GJB2 promoter, (iv) a miRNA regulatory target site (miRTS) for a microRNA expressed in an inner ear cell, (v) a 3′ UTR, and (vi) the 3′ ITR.

In some aspects, the construct comprises a polynucleotide encoding a polypeptide operably linked to an inner ear supporting cell selective promoter and a minimal GJB2 promoter, wherein the construct comprises a miRNA regulatory target site (miRTS) for a microRNA expressed in an inner ear cell.

In some aspects, the construct comprises (i) a 5′ inverted terminal repeat (ITR), (ii) the polynucleotide encoding a polypeptide operably linked to an inner ear supporting cell selective promoter and a minimal GJB2 promoter, (iii) the miRNA regulatory target site (miRTS) for a microRNA expressed in an inner ear cell, and (iv) a 3′ ITR.

In some aspects, the construct comprises (i) a 5′ inverted terminal repeat (ITR), (ii) a 5′ untranslated region (UTR), (iii) the polynucleotide encoding a polypeptide operably linked to an inner ear supporting cell selective promoter and a minimal GJB2 promoter, (iv) the miRNA regulatory target site (miRTS) for a microRNA expressed in an inner ear cell, and (v) a 3′ ITR.

In some aspects, the construct comprises (i) a 5′ inverted terminal repeat (ITR), (ii) the polynucleotide encoding a polypeptide operably linked to an inner ear supporting cell selective promoter and a minimal GJB2 promoter, (iii) the miRNA regulatory target site (miRTS) for a microRNA expressed in an inner ear cell, (iv) a 3′ untranslated region (UTR) and (v) a 3′ ITR.

In some aspects, the construct comprises (i) a 5′ inverted terminal repeat (ITR), (ii) a 5′ untranslated region (UTR), (iii) the polynucleotide encoding a polypeptide operably linked to an inner ear supporting cell selective promoter and a minimal GJB2 promoter, (iv) the miRNA regulatory target site (miRTS) for a microRNA expressed in an inner ear cell, (v) a 3′ UTR, and (vi) a 3′ ITR.

### AAV Particles

Among other things, the present disclosure provides AAV particles that comprise a construct encoding a therapeutic polypeptide, and a capsid described herein. Among other things, the present disclosure provides AAV particles that comprise a construct comprising a nucleic acid sequence (e.g., a gene) encoding a polypeptide, and a capsid described herein. In some aspects, AAV particles can be described as having a serotype, which is a description of the construct strain and the capsid strain. In some aspects, the AAV particle has an AAV1, AAV2, AAV3 (e.g., AAV3B), AAV4, AAV5, AAV6, AAV7, AAV8, AAV9, AAV10, AAV11, AAV2-tYF, AAV2-P2V2, AAV2-P2V3, AAV2-MeStYFTV, AAV2-MeB, AAV2-P2V6, AAV2-DGEDF, or an AAV Anc80 serotype. In some aspects, the AAV particle has an AAVAnc80 serotype (including, for example, an AAVAnc80L65). In some aspects an AAV particle may be described as AAV2, wherein the particle has an AAV2 capsid and a construct that comprises characteristic AAV2 Inverted Terminal Repeats (ITRs). In some aspects, an AAV particle may be described as a pseudotype, wherein the capsid and construct are derived from different AAV strains, for example, AAV2/9 would refer to an AAV particle that comprises a construct utilizing the AAV2 ITRs and an AAV9 capsid.

### AAV Construct

The present disclosure provides constructs that comprise a nucleic acid sequence (e.g., a gene) encoding a polypeptide or characteristic portion thereof. In some aspects described herein, a construct comprising a nucleic acid sequence (e.g., a gene) encoding a polypeptide or characteristic portion thereof can be included in an AAV particle.

The present disclosure provides polynucleotide constructs that comprise a nucleic acid sequence (e.g., a gene) encoding a therapeutic polypeptide or characteristic portion thereof). In some aspects described herein, a polynucleotide comprising a nucleic acid sequence (e.g., a gene) encoding a therapeutic polypeptide or characteristic portion thereof can be included in an AAV particle.

In some aspects, a polynucleotide construct comprises one or more components derived from or modified from naturally occurring AAV genomic construct. In some aspects, a sequence derived from an AAV construct is an AAV1 construct, an AAV2 construct, an AAV3 construct, an AAV4 construct, an AAV5 construct, an AAV6 construct, an AAV7 construct, an AAV8 construct, an AAV9 construct, an AAV2.7m8 construct, an AAV8BP2 construct, an AAV293 construct, an AAV2-tYF construct, an AAV2-P2V2 construct, an AAV2-P2V3 construct, an AAV2-MeStYFTV construct, an AAV2-MeB construct, an AAV2-P2V6 construct, an AAV2-DGEDF construct, or AAV Anc80 construct. In some aspects, the construct is derived from an AAV Anc80 construct (including, for example, an AAVAnc80L65). Additional exemplary AAV constructs that can be used herein are known in the art. See, e.g., Kanaan et al., Mol. Ther. Nucleic Acids 8:184-197, 2017; Li et al., Mol. Ther. 16(7): 1252-1260, 2008; Adachi et al., Nat. Commun. 5: 3075, 2014; Isgrig et al., Nat. Commun. 10(1): 427, 2019; and Gao et al., J. Virol. 78(12): 6381-6388, 2004; each of which is incorporated in its entirety herein by reference.

In some aspects, provided constructs comprise coding sequence, e.g., a nucleic acid encoding polypeptide (e.g., a therapeutic polypeptide), one or more regulatory and/or control sequences, and optionally 5′ and 3′ AAV derived inverted terminal repeats (ITRs). In some aspects wherein a 5′ and 3′ AAV derived ITR is utilized, the polynucleotide construct may be referred to as a recombinant AAV (rAAV) construct. In some aspects, provided rAAV constructs are packaged into an AAV capsid to form an AAV particle. In some aspects, an AAV capsid is an Anc80 capsid (e.g., an Anc80L65 capsid).

In some aspects, AAV derived sequences (which are comprised in a polynucleotide construct) typically include the cis-acting 5′ and 3′ ITR sequences (see, e.g., B. J. Carter, in “Handbook of Parvoviruses,” ed., P. Tijsser, CRC Press, pp. 155 168, 1990, which is incorporated herein by reference in its entirety). Typical AAV2-derived ITR sequences are about 145 nucleotides in length. In some aspects, at least 75% of a typical ITR sequence (e.g., at least 80%, at least 85%, at least 90%, or at least 95%) is incorporated into a construct provided herein. The ability to modify these ITR sequences is within the skill of the art. (See, e.g., texts such as Sambrook et al., “Molecular Cloning. A Laboratory Manual”, 2d ed., Cold Spring Harbor Laboratory, New York, 1989; and K. Fisher et al., J Virol. 70:520 532, 1996, each of which is incorporated in its entirety by reference). In some aspects, any of the coding sequences and/or constructs described herein are flanked by 5′ and 3′ AAV ITR sequences. The AAV ITR sequences may be obtained from any known AAV, including presently identified AAV types.

In some aspects, polynucleotide constructs described in accordance with this disclosure and in a pattern known to the art (see, e.g., Asokan et al., Mol. Ther. 20: 699-7080, 2012, which is incorporated herein by reference in its entirety) are typically comprised of, a coding sequence or a portion thereof, at least one and/or control sequence, and optionally 5′ and 3′ AAV inverted terminal repeats (ITRs). In some aspects, provided constructs can be packaged into a capsid to create an AAV particle. An AAV particle may be delivered to a selected target cell. In some aspects, provided constructs comprise an additional optional coding sequence that is a nucleic acid sequence (e.g., inhibitory nucleic acid sequence), heterologous to the construct sequences, which encodes a polypeptide, protein, functional RNA molecule (e.g., miRNA, miRNA inhibitor) or other gene product, of interest. In some aspects, a nucleic acid coding sequence is operatively linked to and/or control components in a manner that permits coding sequence transcription, translation, and/or expression in a cell of a target tissue.

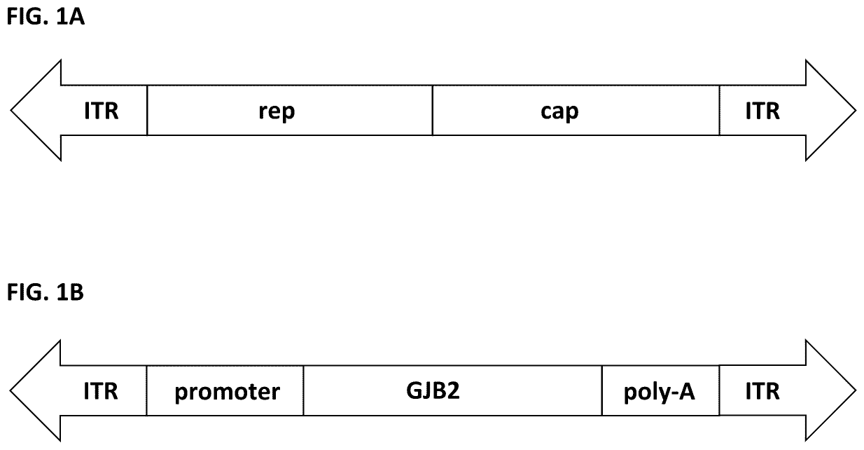

As shown in FIG. 1A, an unmodified AAV endogenous genome includes two open reading frames, “cap” and “rep,” which are flanked by ITRs. As shown in FIG. 1B, exemplary rAAV constructs similarly include ITRs flanking a coding region, e.g., a coding sequence (e.g., a polynucleotide encoding a polypeptide (e.g., a therapeutic polypeptide)). In some aspects, a rAAV construct also comprises conventional control elements that are operably linked to the coding sequence in a manner that permits its transcription, translation and/or expression in a cell transfected with the plasmid construct or infected with the virus produced by the disclosure. In some aspects, a rAAV construct optionally comprises a promoter (shown in FIG. 1, panel (B)), an enhancer, an untranslated region (e.g., a 5′ UTR, 3′ UTR), a Kozak sequence, an internal ribosomal entry site (IRES), splicing sites (e.g., an acceptor site, a donor site), a polyadenylation site (shown in FIG. 1, panel (B)), or any combination thereof. In some aspects, an rAAV construct comprises a promoter, a 5′ UTR, and a polyadenylation site. In some aspects, an rAAV construct comprises a promoter, a 5′ UTR, a 3′ UTR, and a polyadenylation site. Such additional elements are described further herein.

In some aspects, a construct is an rAAV construct. In some aspects, an rAAV construct can include at least 500 bp, at least 1 kb, at least 1.5 kb, at least 2 kb, at least 2.5 kb, at least 3 kb, at least 3.5 kb, at least 4 kb, or at least 4.5 kb. In some aspects, an AAV construct can include at most 7.5 kb, at most 7 kb, at most 6.5 kb, at most 6 kb, at most 5.5 kb, at most 5 kb, at most 4.5 kb, at most 4 kb, at most 3.5 kb, at most 3 kb, or at most 2.5 kb. In some aspects, an AAV construct can include about 1 kb to about 2 kb, about 1 kb to about 3 kb, about 1 kb to about 4 kb, about 1 kb to about 5 kb, about 2 kb to about 3 kb, about 2 kb to about 4 kb, about 2 kb to about 5 kb, about 3 kb to about 4 kb, about 3 kb to about 5 kb, or about 4 kb to about 5 kb.

Any of the constructs described herein can further include regulatory and/or control sequences, e.g., a control sequence selected from the group of a transcription initiation sequence, a transcription termination sequence, a promoter sequence, an enhancer sequence, an RNA splicing sequence, a polyadenylation (poly(A)) sequence, a Kozak consensus sequence, and/or any combination thereof. In some aspects, a promoter can be a native promoter, a constitutive promoter, an inducible promoter, and/or a tissue-specific promoter. Non-limiting examples of control sequences are described herein.

In some aspects, an adeno-assocciated virus (AAV) particle (e.g., an Anc80 particle) comprises a construct comprising: (i) a 5′ inverted terminal repeat (ITR), (ii) a polynucleotide encoding a polypeptide operably linked to a promoter which expresses the polynucleotide in an inner ear support cell, and (iii) a 3′ ITR, wherein the promoter is heterologous to the polynucleotide. In some aspects, the promoter comprises a nucleic acid sequence having at least 85%, at least 90%, at least 95%, at least 96%, at least 97%, at least 98%, at least 99%, or 100% identity to any one of SEQ ID NOs: 16, 28, 40, 57, or 90-99. In some aspects, the construct further comprises a minimal GJB2 promoter. In some aspects, the minimal GJB2 promoter comprises a nucleic acid sequence at least 80%, at least 85%, at least 90%, at least 95%, at least 96%, at least 97%, at least 98%, at least 99%, or 100% identity to SEQ ID NO: 86.

In some aspects, an adeno-assocciated virus (AAV) particle particle (e.g., an Anc80 particle) comprises a construct comprising: (i) a 5′ inverted terminal repeat (ITR), (ii) a 5′ untranslated region (UTR), (iii) a polynucleotide encoding a polypeptide operably linked to a promoter which expresses the polynucleotide in an inner ear support cell, (iv) a 3′ UTR, and (v) a 3′ ITR, wherein the promoter is heterologous to the polynucleotide.

In some aspects, an adeno-associated virus (AAV) particle particle (e.g., an Anc80 particle) comprises a construct comprising: (i) a 5′ inverted terminal repeat (ITR), (ii) a 5′ untranslated region (UTR), (iii) a polynucleotide encoding a polypeptide operably linked to a promoter, (iv) a 3′ UTR, and (v) a 3′ ITR, wherein the promoter comprises a nucleic acid sequence having at least 85%, at least 90%, at least 95%, at least 96%, at least 97%, at least 98%, at least 99%, or 100% identity to any one of SEQ ID NOs: 16, 28, 40, 57, or 90-99.

In some aspects, an adeno-associated virus (AAV) particle (e.g., an Anc80 particle) comprises a construct comprising: (i) a 5′ inverted terminal repeat (ITR), (ii) a 5′ untranslated region (UTR), (iii) a polynucleotide encoding a polypeptide operably linked to a promoter, (iv) a miRNA regulatory target site (miRTS) for a microRNA expressed in an inner ear cell, (v) a 3′ UTR, and (vi) a 3′ ITR.

In some aspects, an adeno-assocciated virus (AAV) particle (e.g., an Anc80 particle) comprises a construct comprising: (i) a 5′ inverted terminal repeat (ITR), (ii) a 5′ untranslated region (UTR), (iii) a polynucleotide encoding a polypeptide operably linked to an inner ear supporting cell selective promoter and a minimal GJB2 promoter which expresses the polynucleotide in an inner ear support cell, (iv) a 3′ UTR, and (v) a 3′ ITR, wherein the inner ear supporting cell selective promoter is heterologous to the polynucleotide.

In some aspects, an adeno-associated virus (AAV) particle (e.g., an Anc80 particle) comprises a construct comprising: (i) a 5′ inverted terminal repeat (ITR), (ii) a 5′ untranslated region (UTR), (iii) a polynucleotide encoding a polypeptide operably linked to an inner ear supporting cell selective promoter and a minimal GJB2 promoter, (iv) a 3′ UTR, and (v) a 3′ ITR, wherein the inner ear supporting cell selective promoter comprises a nucleic acid sequence having at least 85%, at least 90%, at least 95%, at least 96%, at least 97%, at least 98%, at least 99%, or 100% identity to any one of SEQ ID NOs: 16, 28, 40, 57, or 90-99.

In some aspects, an adeno-associated virus (AAV) particle (e.g., an Anc80 particle) comprises a construct comprising: (i) a 5′ inverted terminal repeat (ITR), (ii) a 5′ untranslated region (UTR), (iii) a polynucleotide encoding a polypeptide operably linked to an inner ear supporting cell selective promoter and a minimal GJB2 promoter, (iv) a miRNA regulatory target site (miRTS) for a microRNA expressed in an inner ear cell, (v) a 3′ UTR, and (vi) a 3′ ITR.

### Exemplary Construct Components

### Inverted Terminal Repeat Sequences (ITRs)

AAV derived sequences of a construct typically comprises the cis-acting 5′ and 3′ ITRs (See, e.g., B. J. Carter, in “Handbook of Parvoviruses”, ed., P. Tijsser, CRC Press, pp. 155 168 (1990), which is incorporated in its entirety herein by reference). Generally, ITRs are able to form a hairpin. The ability to form a hairpin can contribute to an ITRs ability to self-prime, allowing primase-independent synthesis of a second DNA strand. ITRs also play a role in integration of AAV construct (e.g., a coding sequence, e.g., a polynucleotide encoding a polypeptide (e.g., a therapeutic polypeptide) into a genome of a subject's cell. ITRs can also aid in efficient encapsidation of an AAV construct in an AAV particle.

An rAAV particle (e.g., an AAV2/Anc80 particle) of the present disclosure can comprise a rAAV construct comprising a coding sequence (e.g., a polynucleotide encoding a polypeptide (e.g., a therapeutic polypeptide)) and associated elements flanked by a 5′ and a 3′ AAV ITR sequences. In some aspects, an ITR is or comprises about 145 nucleic acids. In some aspects, an ITR is or comprises about 119 nucleic acids. In some aspects, an ITR is or comprises about 130 nucleic acids. In some aspects, all or substantially all of a sequence encoding an ITR is used. An AAV ITR sequence may be obtained from any known AAV, including presently identified mammalian AAV types. In some aspects an ITR is an AAV2 ITR.

An example of a construct molecule employed in the present disclosure is a “cis-acting” construct containing a transgene, in which the selected transgene sequence and associated regulatory elements are flanked by 5′ or “left” and 3′ or “right” AAV ITR sequences. 5′ and left designations refer to a position of an ITR sequence relative to an entire construct, read left to right, in a sense direction. For example, in some aspects, a 5′ or left ITR is an ITR that is closest to a promoter (as opposed to a polyadenylation sequence) for a given construct, when a construct is depicted in a sense orientation, linearly. Concurrently, 3′ and right designations refer to a position of an ITR sequence relative to an entire construct, read left to right, in a sense direction. For example, in some aspects, a 3′ or right ITR is an ITR that is closest to a polyadenylation sequence (as opposed to a promoter sequence) for a given construct, when a construct is depicted in a sense orientation, linearly. ITRs as provided herein are depicted in 5′ to 3′ order in accordance with a sense strand. Accordingly, one of skill in the art will appreciate that a 5′ or “left” orientation ITR can also be depicted as a 3′ or “right” ITR when converting from sense to antisense direction. Further, it is well within the ability of one of skill in the art to transform a given sense ITR sequence (e.g., a 5′/left AAV ITR) into an antisense sequence (e.g., 3′/right ITR sequence). One of ordinary skill in the art would understand how to modify a given ITR sequence for use as either a 5′/left or 3′/right ITR, or an antisense version thereof.

For example, in some aspects an ITR (e.g., a 5′ ITR) can have a sequence according to SEQ ID NO: 8. In some aspects, an ITR (e.g., a 3′ ITR) can have a sequence according to SEQ ID NO: 9. In some aspects, an ITR includes one or more modifications, e.g., truncations, deletions, substitutions or insertions, as is known in the art. In some aspects, an ITR comprises fewer than 145 nucleotides, e.g., 119, 127, 130, 134 or 141 nucleotides. For example, in some aspects, an ITR comprises 110, 111, 112, 113, 114, 115, 116, 117, 118, 119, 120, 121, 122, 123,124, 125, 126, 127, 128, 129, 130, 131, 132, 133, 134, 135, 136, 137, 138, 139, 140, 141, 142, 143 144, or 145 nucleotides. In some aspects, the ITR comprises about 119 nucleotides. In some aspects, the ITR comprises about 130 nucleotides. In some aspects an ITR (e.g., a 5′ ITR) can have a sequence according to SEQ ID NO: 52. In some aspects, an ITR (e.g., a 3′ ITR) can have a sequence according to SEQ ID NO: 53.

A non-limiting example of 5′ AAV ITR sequences includes SEQ ID NO: 8 or 52. A non-limiting example of 3′ AAV ITR sequences includes SEQ ID NO: 9 or 53. In some aspects, the 5′ and a 3′ AAV ITRs (e.g., SEQ ID NOs: 8 and 9, or SEQ ID NOs: 52 and 53) flank a portion of a coding sequence, e.g., all or a portion of a polynucleotide encoding a polypeptide (e.g., a therapeutic polypeptide). The ability to modify these ITR sequences is within the skill of the art. (See, e.g., texts such as Sambrook et al. “Molecular Cloning. A Laboratory Manual”, 2d ed., Cold Spring Harbor Laboratory, New York (1989); and K. Fisher et al., J Virol., 70:520 532 (1996), each of which is incorporated in its entirety herein by reference). In some aspects, a 5′ ITR sequence is at least at least 85%, at least 90%, at least 95%, at least 96%, at least 97%, at least 98% or at least 99%, or 100% identical to a 5′ ITR sequence represented by SEQ ID NO: 8. In some aspects, a 3′ ITR sequence is at least at least 85%, at least 90%, at least 95%, at least 96%, at least 97%, at least 98% at least 99%, or 100% identical to a 3′ ITR sequence represented by SEQ ID NO: 9. In some aspects, a 5′ ITR sequence is at least at least 85%, at least 90%, at least 95%, at least 96%, at least 97%, at least 98% at least 99%, or 100% identical to a 5′ ITR sequence represented by SEQ ID NO: 52. In some aspects, a 3′ ITR sequence is at least 85%, at least 90%, at least 95%, at least 96%, at least 97%, at least 98% at least 99%, or 100% identical to a 3′ ITR sequence represented by SEQ ID NO: 53.

In some aspects, a 3′ ITR sequence is at least at least 85%, at least 90%, at least 95%, at least 96%, at least 97%, at least 98% at least 99%, or 100% identical to a 3′ ITR sequence represented by SEQ ID NO: 116. In some aspects, a 3′ ITR sequence is at least 85%, at least 90%, at least 95%, at least 96%, at least 97%, at least 98% at least 99%, or 100% identical to a 3′ ITR sequence represented by SEQ ID NO: 116.

Exemplary 5′ AAV ITR
(SEQ ID NO: 8)
TTGGCCACTCCCTCTCTGCGCGCTCGCTCGCTCACTGAGGCCGCC
CGGGCAAAGCCCGGGCGTCGGGCGACCTTTGGTCGCCCGGCCTCA
GTGAGCGAGCGAGCGCGCAGAGAGGGAGTGGCCAACTCCATCACT
AGGGGTTCCT
Exemplary 3′ AAV ITR
(SEQ ID NO: 9)
AGGAACCCCTAGTGATGGAGTTGGCCACTCCCTCTCTGCGCGCTC
GCTCGCTCACTGAGGCCGGGCGACCAAAGGTCGCCCGACGCCCGG
GCTTTGCCCGGGCGGCCTCAGTGAGCGAGCGAGCGCGCAGAGAGG
GAGTGGCCAA
Exemplary 5′ AAV ITR
(SEQ ID NO: 52)
CTGCGCGCTCGCTCGCTCACTGAGGCCGCCCGGGCGTCGGGCGAC
CTTTGGTCGCCCGGCCTCAGTGAGCGAGCGAGCGCGCAGAGAGGG
AGTGGCCAACTCCATCACTAGGGGTTCCT
Exemplary 3′ AAV ITR
(SEQ ID NO: 53)
AGGAACCCCTAGTGATGGAGTTGGCCACTCCCTCTCTGCGCGCTC
GCTCGCTCACTGAGGCCGGGCGACCAAAGGTCGCCCGACGCCCGG
GCTTTGCCCGGGCGGCCTCAGTGAGCGAGCGAGCGCGCAG
Exemplary 3′ ITR
(SEQ ID NO: 116)
AGGAACCCCTAGTGATGGAGTTGGCCACTCCCTCTCTGCGCGCTC
GCTCGCTCACTGAGGCCGCCCGGGCAAAGCCCGGGCGTCGGGCGA
CCTTTGGTCGCCCGGCCTCAGTGAGCGAGCGAGCGCGCAGAGAGG
GAGTGGCCAA

### Promoters

In some aspects, the disclosure is directed to constructs comprising a cell selective promoter which can be used to regulate (e.g., increase) expression of a polynucleotide encoding a therapeutic polypeptide in a cell (e.g., an inner ear cell, e.g., a supporting cell). In some aspects, the constructs provide reduced toxicity associated with expression of the therapeutic polypeptide in some cells (e.g., an inner ear cell, e.g., a hair cell).

In some aspects, the disclosure is directed to constructs comprising a cell selective promoter which can be used to regulate (e.g., increase) expression of a polynucleotide encoding a polypeptide in a cell (e.g., an inner ear cell, e.g., a supporting cell). In some aspects, the constructs provide reduced toxicity associated with expression of the polypeptide in some cells (e.g., an inner ear cell, e.g., a hair cell).

In some aspects, a construct (e.g., an rAAV construct) comprises a promoter. The term “promoter” refers to a DNA sequence recognized by enzymes/proteins that can promote and/or initiate transcription of an operably linked gene (e.g., a polynucleotide encoding a polypeptide (e.g., a therapeutic polypeptide)). For example, a promoter typically refers to, e.g., a nucleotide sequence to which an RNA polymerase and/or any associated factor binds and from which it can initiate transcription. Thus, in some aspects, a construct (e.g., an rAAV construct) comprises a polynucleotide operably linked to one of the non-limiting example promoters described herein.

In some aspects, a promoter is an inducible promoter, a constitutive promoter, a mammalian cell promoter, a viral promoter, a chimeric promoter, an engineered promoter, a tissue-specific promoter, a cell-selective promoter or any other type of promoter known in the art. In some aspects, a promoter is a RNA polymerase II promoter, such as a mammalian RNA polymerase II promoter. In some aspects, a promoter is a RNA polymerase III promoter, including, but not limited to, a HI promoter, a human U6 promoter, a mouse U6 promoter, or a swine U6 promoter. A promoter will generally be one that is able to promote transcription in an inner ear cell. In some aspects, a promoter is a cochlea-selective promoter or a cochlea-oriented promoter. In some aspects, a promoter is a hair cell selective promoter, or a supporting cell selective promoter. In some aspects, a promoter is an inner ear supporting cell selective promoter.

The term “constitutive” promoter refers to a nucleotide sequence that, when operably linked with a nucleic acid encoding a protein (e.g., a polypeptide (e.g., a therapeutic polypeptide)), causes RNA to be transcribed from the nucleic acid in a cell under most or all physiological conditions.

Examples of constitutive promoters include, without limitation, the retroviral Rous sarcoma virus (RSV) LTR promoter, the cytomegalovirus (CMV) promoter (see, e.g., Boshart et al., Cell 41:521-530, 1985, which is incorporated in its entirety herein by reference), the SV40 promoter, the dihydrofolate reductase promoter, the beta-actin promoter, the phosphoglycerol kinase (PGK) promoter, and the EF1-alpha promoter (Invitrogen). In some aspects, the promoter is a constitutive promoter. In some aspects, the constitutive promoter is a CAG promoter, a CBA promoter, a CMV promoter, a CMV/CBA enhancer/promoter, or a CB7 promoter. In some aspects, the a CMV/CBA enhancer/promoter comprises a nucleic acid with at least 85%, at least 90%, at least 95%, at least 96%, at least 97%, at least 98% at least 99%, or 100% identity to SEQ ID NOs: 12 or 13. In some aspects, the CMV/CBA enhancer/promoter comprises a nucleic acid of SEQ ID NO: 12. In some aspects, the CMV/CBA enhancer/promoter comprises a nucleic acid of SEQ ID NO: 13. In some aspects, the CBA promoter comprises a nucleic acid with at least 85%, at least 90%, at least 95%, at least 96%, at least 97%, at least 98% at least 99%, or 100% identity to SEQ ID NOs: 10 or 11. In some aspects, the CBA promoter comprises a nucleic acid of SEQ ID NO: 10. In some aspects, the CBA promoter comprises a nucleic acid of SEQ ID NO: 11.

In some aspects, the CAG promoter comprises a nucleic acid with at least 85%, at least 90%, at least 95%, at least 96%, at least 97%, at least 98% at least 99%, or 100% identity to SEQ ID NOs: 14 or 15. In some aspects, the CAG promoter comprises a nucleic acid of SEQ ID NO: 14. In some aspects, the CAG promoter comprises a nucleic acid of SEQ ID NO: 15.

In some aspects, regulatory and/or control sequences impart cell selective gene expression capabilities. In some cases, cell selective regulatory and/or control sequences bind cell selective transcription factors that induce transcription in a cell selective manner.

In some aspects, a cell selective promoter is an ear cell selective promoter. In some aspects, a cell selective promoter is an inner ear cell selective promoter. In some aspects, a promoter is a characteristic fragment of a cell selective promoter. In some aspects, the promoter is an inner ear supporting cell selective promoter.

In some aspects, the inner ear supporting cells are selected from one or more of inner phalangeal cells/border cells (IPhC), inner pillar cells (IPC), outer pillar cells (OPC), Deiters' cells rows 1 and 2 (DC1/2), Deiters' cells row 3 (DC3), Hensen's cells (Hec), Claudius cells/outer sulcus cells (CC/OSC), interdental cells (Idc), inner sulcus cells (ISC), KöHiker's organ cells (KO), greater ridge epithelial ridge cells (GER) (including lateral greater epithelial ridge cells (LGER)), and OC90+ cells (OC90), fibroblasts, and other cells of the lateral wall.

In some aspects, inner ear support cell selective promoters are selected from one or more of GJB2, GJB6, IGFBP2, RBP7, GDF6, PARM1, GFAP, BACE2, DBI2, FABP3, KLHL14, MMP15, SPARC, TSPAN8, VIM, derivatives thereof, or fragments thereof. In some aspects, derivatives thereof can include a modified parent sequence (e.g., a naturally occuring promoter sequence), one or more portions of a parent sequence, fragments of a parent sequence, and the like. In some aspects, the promoter is an inner ear medial support cell selective promoter. In some aspects, inner ear medial support cells are selected from one or more of lateral greater epithelial ridge cells and inner sulcus cells. In some aspects, inner ear medial support cell selective promoters are selected from one or more of GJB6, IGFBP2, GDF6, PARM1, derivatives thereof, or fragments thereof. In some aspects, the promoter is an inner ear sensory epithelial support cell selective promoter. In some aspects, sensory epithelial support cells are selected from one or more of inner pillar cells, outer pillar cells, dieter cells, and inner phalangeal cells. In some aspects, inner ear sensory epithelial support cell selective promoters are selected from one or more of GJB6, IGFBP2, RBP7, GDF6, PARM1, FABP3, BACE2 derivatives thereof, or fragments thereof. In some aspects, the promoter is an inner phalangeal cell selective promoter. In some aspects, the inner phalangeal cell selective promoters are selected from one or more of IGFBP2, GDF6, FABP3, BACE2, derivatives thereof, or fragments thereof. In some aspects, the promoter is an interdental cell selective promoter. In some aspects, the interdental cell promoter is IGFBP2, derivative thereof, or fragment thereof.

In some aspects, the inner ear supporting cell selective promoter is a GJB2 promoter. In some aspects, the GJB2 enhancer comprises a nucleic acid sequence with at least 85%, at least 90%, at least 95%, at least 96%, at least 97%, at least 98% at least 99%, or 100% identity to SEQ ID NO: 65. In some aspects, the GJB2 enhancer comprises the nucleic acid sequence of SEQ ID NO: 65. In some aspects, the GJB2 minimal promoter comprises a nucleic acid sequence with at least 85%, at least 90%, at least 95%, at least 96%, at least 97%, at least 98% at least 99%, or 100% identity to SEQ ID NO: 86. In some aspects, the GJB2 minimal promoter comprises the nucleic acid sequence of SEQ ID NO: 86.

In some aspects, the promoter is derived from a GJB2 promoter and has a length of 1000-1050 nucleotides. In some aspects, the inner ear supporting cell selective promoter is a GJB6 promoter. In some aspects, the GJB6 promoter comprises a nucleic acid sequence with at least 85%, at least 90%, at least 95%, at least 96%, at least 97%, at least 98% at least 99%, or 100% identity to SEQ ID NO: 16. In some aspects, the GJB6 promoter comprises the nucleic acid sequence of SEQ ID NO: 16. In some aspects, the promoter is derived from a GJB6 promoter and has a length of 700-750 nucleotides.

In some aspects, the inner ear supporting cell selective promoter is an IGFBP2 promoter. In some aspects, the IGFBP2 promoter comprises a nucleic acid sequence with at least 85%, at least 90%, at least 95%, at least 96%, at least 97%, at least 98% at least 99%, or 100% identity to SEQ ID NO: 57. In some aspects, the IGFBP2 promoter comprises the nucleic acid sequence of SEQ ID NO: 57. In some aspects, the promoter is derived from an IGFBP2 promoter and has a length of 1500-1550 nucleotides.

In some aspects, the inner ear supporting cell selective promoter is a RBP7 promoter. In some aspects, the RBP7 promoter comprises a nucleic acid sequence with at least 85%, at least 90%, at least 95%, at least 96%, at least 97%, at least 98% at least 99%, or 100% identity to SEQ ID NO: 28. In some aspects, the RBP7 promoter comprises the nucleic acid sequence of SEQ ID NO: 28. In some aspects, the promoter is derived from a RBP7 promoter and has a length of 1050-1100 nucleotides.

In some aspects, the inner ear supporting cell selective promoter is a GDF6 promoter. In some aspects, the GDF6 promoter comprises a nucleic acid sequence with at least 85%, at least 90%, at least 95%, at least 96%, at least 97%, at least 98% at least 99%, or 100% identity to SEQ ID NO: 90. In some aspects, the GDF6 promoter comprises the nucleic acid sequence of SEQ ID NO: 90. In some aspects, the promoter is derived from a GDF6 promoter and has a length of 1150-1200 nucleotides.

In some aspects, the inner ear supporting cell selective promoter is a PARM1 promoter. In some aspects, the PARM1 promoter comprises a nucleic acid sequence with at least 85%, at least 90%, at least 95%, at least 96%, at least 97%, at least 98% at least 99%, or 100% identity to SEQ ID NO: 40. In some aspects, the PARM1 promoter comprises the nucleic acid sequence of SEQ ID NO: 40. In some aspects, the promoter is derived from a PARM1 promoter and has a length of 1300-1350 nucleotides.

In some aspects, the construct comprises two or more promoters. In some aspects, the first promoter is selected from a GJB6 promoter, a GDF6 promoter, a IGFBP2 promoter, a RBP7 promoter, a PARM1 promoter, a GFAP promoter, a BACE2 promoter, a DBI2 promoter, a FABP3 promoter, a KLHL14 promoter, a MMP15 promoter, a SPARC promoter, a TSPAN8 promoter, a VIM promoter, and any combination thereof. In some aspects, the second promoter is selected from a GJB2 promoter or a minimal GJB2 promoter.

In some aspects, the inner ear supporting cell selective promoter comprises a GJB6 and a hGJB2 minimal promoter. In some aspects, the GJB6 promoter comprises a nucleic acid sequence with at least 85%, at least 90%, at least 95%, at least 96%, at least 97%, at least 98% at least 99%, or 100% identity to SEQ ID NO: 16 and the hGJB2 minimal promoter comprises a nucleic acid sequence with at least 85%, at least 90%, at least 95%, at least 96%, at least 97%, at least 98% at least 99%, or 100% to SEQ ID NO: 86. In some aspects, the GJB6 has the nucleic acid sequence of SEQ ID NO: 16 and the hGJB2 minimal promoter has the nucleic acid sequence of SEQ ID NO: 86.

In some aspects, the inner ear supporting cell selective promoter comprises a IGFBP2 promoter and a hGJB2 minimal promoter. In some aspects, the IGFBP2 promoter comprises a nucleic acid sequence with at least 85%, at least 90%, at least 95%, at least 96%, at least 97%, at least 98% at least 99%, or 100% identity to SEQ ID NO: 57 and the hGJB2 minimal promoter comprises a nucleic acid sequence with at least 85%, at least 90%, at least 95%, at least 96%, at least 97%, at least 98% at least 99%, or 100% to SEQ ID NO: 86. In some aspects, the IGFBP2 has the nucleic acid sequence of SEQ ID NO: 57 and the hGJB2 minimal promoter has the nucleic acid sequence of SEQ ID NO: 86.

In some aspects, the inner ear supporting cell selective promoter comprises a RBP7 promoter and a hGJB2 minimal promoter. In some aspects, the RBP7 promoter comprises a nucleic acid sequence with at least 85%, at least 90%, at least 95%, at least 96%, at least 97%, at least 98% at least 99%, or 100% identity to SEQ ID NO: 28 and the hGJB2 minimal promoter comprises a nucleic acid sequence with at least 85%, at least 90%, at least 95%, at least 96%, at least 97%, at least 98% at least 99%, or 100% to SEQ ID NO: 86. In some aspects, the RBP7 has the nucleic acid sequence of SEQ ID NO: 28 and the hGJB2 minimal promoter has the nucleic acid sequence of SEQ ID NO: 86.

In some aspects, the inner ear supporting cell selective promoter comprises a GJB6 promoter and a hGJB2 minimal promoter. In some aspects, the GJB6 promoter comprises a nucleic acid sequence with at least 85%, at least 90%, at least 95%, at least 96%, at least 97%, at least 98% at least 99%, or 100% identity to SEQ ID NO: 16 and the hGJB2 minimal promoter comprises a nucleic acid sequence with at least 85%, at least 90%, at least 95%, at least 96%, at least 97%, at least 98% at least 99%, or 100% to SEQ ID NO: 86. In some aspects, the GJB6 has the nucleic acid sequence of SEQ ID NO: 16 and the hGJB2 minimal promoter has the nucleic acid sequence of SEQ ID NO: 86.

In some aspects, the inner ear supporting cell selective promoter comprises a PARM1 promoter and a hGJB2 minimal promoter. In some aspects, the PARM1 promoter comprises a nucleic acid sequence with at least 85%, at least 90%, at least 95%, at least 96%, at least 97%, at least 98% at least 99%, or 100% identity to SEQ ID NO: 40 and the hGJB2 minimal promoter comprises a nucleic acid sequence with at least 85%, at least 90%, at least 95%, at least 96%, at least 97%, at least 98% at least 99%, or 100% to SEQ ID NO: 86. In some aspects, the PARM1 has the nucleic acid sequence of SEQ ID NO: 40 and the hGJB2 minimal promoter has the nucleic acid sequence of SEQ ID NO: 86.

In some aspects, the inner ear supporting cell selective promoter is a BACE2 promoter. In some aspects, the BACE2 promoter comprises a nucleic acid sequence with at least 85%, at least 90%, at least 95%, at least 96%, at least 97%, at least 98% at least 99%, or 100% identity to SEQ ID NO: 92. In some aspects, the BACE2 promoter comprises the nucleic acid sequence of SEQ ID NO: 92. In some aspects, the promoter is derived from a BACE2 promoter and has a length of 1400-1450 nucleotides.

In some aspects, the inner ear supporting cell selective promoter is a DBI2 promoter. In some aspects, the DBI2 promoter comprises a nucleic acid sequence with at least 85%, at least 90%, at least 95%, at least 96%, at least 97%, at least 98% at least 99%, or 100% identity to SEQ ID NO: 93. In some aspects, the DBI2 promoter comprises the nucleic acid sequence of SEQ ID NO: 93. In some aspects, the promoter is derived from a DBI2 promoter and has a length of 1450-1500 nucleotides.

In some aspects, the inner ear supporting cell selective promoter is a FABP3 promoter. In some aspects, the FABP3 promoter comprises a nucleic acid sequence with at least 85%, at least 90%, at least 95%, at least 96%, at least 97%, at least 98% at least 99%, or 100% identity to SEQ ID NO: 94. In some aspects, the FABP3 promoter comprises the nucleic acid sequence of SEQ ID NO: 94. In some aspects, the promoter is derived from a FABP3 promoter and has a length of 1750-1800 nucleotides.

In some aspects, the inner ear supporting cell selective promoter is a KLHL14 promoter. In some aspects, the KLHL14 promoter comprises a nucleic acid sequence with at least 85%, at least 90%, at least 95%, at least 96%, at least 97%, at least 98% at least 99%, or 100% identity to SEQ ID NO: 95. In some aspects, the KLHL14 promoter comprises the nucleic acid sequence of SEQ ID NO: 95. In some aspects, the promoter is derived from a KLHL14 promoter and has a length of 1250-1300 nucleotides.

In some aspects, the inner ear supporting cell selective promoter is a MMP15 promoter. In some aspects, the MMP15 promoter comprises a nucleic acid sequence with at least 85%, at least 90%, at least 95%, at least 96%, at least 97%, at least 98% at least 99%, or 100% identity to SEQ ID NO: 96. In some aspects, the MMP15 promoter comprises the nucleic acid sequence of SEQ ID NO: 96. In some aspects, the promoter is derived from a MMP15 promoter and has a length of 1000-1050 nucleotides.

In some aspects, the inner ear supporting cell selective promoter is a SPARC promoter. In some aspects, the SPARC promoter comprises a nucleic acid sequence with at least 85%, at least 90%, at least 95%, at least 96%, at least 97%, at least 98% at least 99%, or 100% identity to SEQ ID NO: 97. In some aspects, the SPARC promoter comprises the nucleic acid sequence of SEQ ID NO: 97. In some aspects, the promoter is derived from a SPARC promoter and has a length of 1000-1050 nucleotides.

In some aspects, the inner ear supporting cell selective promoter is a TSPAN8 promoter. In some aspects, the TSPAN8 promoter comprises a nucleic acid sequence with at least 85%, at least 90%, at least 95%, at least 96%, at least 97%, at least 98% at least 99%, or 100% identity to SEQ ID NO: 98. In some aspects, the TSPAN8 promoter comprises the nucleic acid sequence of SEQ ID NO: 98. In some aspects, the promoter is derived from a TSPAN8 promoter and has a length of 1200-1250 nucleotides.

In some aspects, the inner ear supporting cell selective promoter is a GFAP promoter. In some aspects, the GFAP promoter comprises a nucleic acid sequence with at least 85%, at least 90%, at least 95%, at least 96%, at least 97%, at least 98% at least 99%, or 100% identity to SEQ ID NO: 91. In some aspects, the GFAP promoter comprises the nucleic acid sequence of SEQ ID NO: 91. In some aspects, the promoter is derived from a GFAP promoter and has a length of 650-700 nucleotides.

In some aspects, the inner ear supporting cell selective promoter is a VIM promoter. In some aspects, the VIM promoter comprises a nucleic acid sequence with at least 85%, at least 90%, at least 95%, at least 96%, at least 97%, at least 98% at least 99%, or 100% identity to SEQ ID NO: 99. In some aspects, the VIM promoter comprises the nucleic acid sequence of SEQ ID NO: 99. In some aspects, the promoter is derived from a VIM promoter and has a length of 1050-1100 nucleotides.

In some aspects, the inner ear supporting cell selective promoter comprises a BACE2 promoter and a hGJB2 minimal promoter. In some aspects, the BACE2 promoter comprises a nucleic acid sequence with at least 85%, at least 90%, at least 95%, at least 96%, at least 97%, at least 98% at least 99%, or 100% identity to SEQ ID NO: 92 and the hGJB2 minimal promoter comprises a nucleic acid sequence with at least 85%, at least 90%, at least 95%, at least 96%, at least 97%, at least 98% at least 99%, or 100% to SEQ ID NO: 86. In some aspects, the BACE2 promoter comprises the nucleic acid sequence of SEQ ID NO: 92 and the hGJB2 minimal promoter has the nucleic acid sequence of SEQ ID NO: 86.

In some aspects, the inner ear supporting cell selective promoter comprises a DBI2 promoter and a hGJB2 minimal promoter. In some aspects, the DBI2 promoter comprises a nucleic acid sequence with at least 85%, at least 90%, at least 95%, at least 96%, at least 97%, at least 98% at least 99%, or 100% identity to SEQ ID NO: 93 and the hGJB2 minimal promoter comprises a nucleic acid sequence with at least 85%, at least 90%, at least 95%, at least 96%, at least 97%, at least 98% at least 99%, or 100% to SEQ ID NO: 86. In some aspects, the DBI2 promoter comprises the nucleic acid sequence of SEQ ID NO: 93 and the hGJB2 minimal promoter has the nucleic acid sequence of SEQ ID NO: 86.

In some aspects, the inner ear supporting cell selective promoter comprises a FABP3 promoter and a hGJB2 minimal promoter. In some aspects, the FABP3 promoter comprises a nucleic acid sequence with at least 85%, at least 90%, at least 95%, at least 96%, at least 97%, at least 98% at least 99%, or 100% identity to SEQ ID NO: 94 and the hGJB2 minimal promoter comprises a nucleic acid sequence with at least 85%, at least 90%, at least 95%, at least 96%, at least 97%, at least 98% at least 99%, or 100% to SEQ ID NO: 86. In some aspects, the FABP3 promoter comprises the nucleic acid sequence of SEQ ID NO: 94 and the hGJB2 minimal promoter has the nucleic acid sequence of SEQ ID NO: 86.

In some aspects, the inner ear supporting cell selective promoter comprises a KLHL14 promoter and a hGJB2 minimal promoter. In some aspects, the KLHL14 promoter comprises a nucleic acid sequence with at least 85%, at least 90%, at least 95%, at least 96%, at least 97%, at least 98% at least 99%, or 100% identity to SEQ ID NO: 95 and the hGJB2 minimal promoter comprises a nucleic acid sequence with at least 85%, at least 90%, at least 95%, at least 96%, at least 97%, at least 98% at least 99%, or 100% to SEQ ID NO: 86. In some aspects, the KLHL14 promoter comprises the nucleic acid sequence of SEQ ID NO: 95 and the hGJB2 minimal promoter has the nucleic acid sequence of SEQ ID NO: 86.

In some aspects, the inner ear supporting cell selective promoter comprises a MMP15 promoter and a hGJB2 minimal promoter. In some aspects, the MMP15 promoter comprises a nucleic acid sequence with at least 85%, at least 90%, at least 95%, at least 96%, at least 97%, at least 98% at least 99%, or 100% identity to SEQ ID NO: 96 and the hGJB2 minimal promoter comprises a nucleic acid sequence with at least 85%, at least 90%, at least 95%, at least 96%, at least 97%, at least 98% at least 99%, or 100% to SEQ ID NO: 86. In some aspects, the MMP15 promoter comprises the nucleic acid sequence of SEQ ID NO: 96 and the hGJB2 minimal promoter has the nucleic acid sequence of SEQ ID NO: 86.

In some aspects, the inner ear supporting cell selective promoter comprises a SPARC promoter and a hGJB2 minimal promoter. In some aspects, the SPARC promoter comprises a nucleic acid sequence with at least 85%, at least 90%, at least 95%, at least 96%, at least 97%, at least 98% at least 99%, or 100% identity to SEQ ID NO: 97 and the hGJB2 minimal promoter comprises a nucleic acid sequence with at least 85%, at least 90%, at least 95%, at least 96%, at least 97%, at least 98% at least 99%, or 100% to SEQ ID NO: 86. In some aspects, the SPARC promoter comprises the nucleic acid sequence of SEQ ID NO: 97 and the hGJB2 minimal promoter has the nucleic acid sequence of SEQ ID NO: 86.

In some aspects, the inner ear supporting cell selective promoter comprises a TSPAN8 promoter and a hGJB2 minimal promoter. In some aspects, the TSPAN8 promoter comprises a nucleic acid sequence with at least 85%, at least 90%, at least 95%, at least 96%, at least 97%, at least 98% at least 99%, or 100% identity to SEQ ID NO: 98 and the hGJB2 minimal promoter comprises a nucleic acid sequence with at least 85%, at least 90%, at least 95%, at least 96%, at least 97%, at least 98% at least 99%, or 100% to SEQ ID NO: 86. In some aspects, the TSPAN8 promoter comprises the nucleic acid sequence of SEQ ID NO: 98 and the hGJB2 minimal promoter has the nucleic acid sequence of SEQ ID NO: 86.

In some aspects, the inner ear supporting cell selective promoter comprises a VIM promoter and a hGJB2 minimal promoter. In some aspects, the VIM promoter comprises a nucleic acid sequence with at least 85%, at least 90%, at least 95%, at least 96%, at least 97%, at least 98% at least 99%, or 100% identity to SEQ ID NO: 99 and the hGJB2 minimal promoter comprises a nucleic acid sequence with at least 85%, at least 90%, at least 95%, at least 96%, at least 97%, at least 98% at least 99%, or 100% to SEQ ID NO: 86. In some aspects, the VIM promoter comprises the nucleic acid sequence of SEQ ID NO: 99 and the hGJB2 minimal promoter has the nucleic acid sequence of SEQ ID NO: 86.

Exemplary CBA promoter
(SEQ ID NO: 10)
GTCGAGGTGAGCCCCACGTTCTGCTTCACTCTCCCCATCTCCCCC
CCCTCCCCACCCCCAATTTTGTATTTATTTATTTTTTAATTATTT
TGTGCAGCGATGGGGGGGGGGGGGGGGGGGGCGCGCGCCAGGCGG
GGCGGGGCGGGGCGAGGGGCGGGGCGGGGCGAGGCGGAGAGGTGC
GGCGGCAGCCAATCAGAGCGGCGCGCTCCGAAAGTTTCCTTTTAT
GGCGAGGCGGCGGCGGCGGCGGCCCTATAAAAAGCGAAGCGCGCG
GCGGGCG
Exemplary CBA promoter
(SEQ ID NO: 11)
GTCGAGGTGAGCCCCACGTTCTGCTTCACTCTCCCCATCTCCCCC
CCCTCCCCACCCCCAATTTTGTATTTATTTATTTTTTAATTATTT
TGTGCAGCGATGGGGGGGGGGGGGGGGGGGGGGCGCGCGCCAGGC
GGGGCGGGGGGGGGCGAGGGGGGGGGGGGGGCGAGGCGGAGAGGT
GCGGCGGCAGCCAATCAGAGCGGCGCGCTCCGAAAGTTTCCTTTT
ATGGCGAGGCGGCGGCGGCGGCGGCCCTATAAAAAGCGAAGCGCG
CGGCGGGCG
Exemplary CMV/CBA enhancer/promoter
(SEQ ID NO: 12)
GACATTGATTATTGACTAGTTATTAATAGTAATCAATTACGGGGT
CATTAGTTCATAGCCCATATATGGAGTTCCGCGTTACATAACTTA
CGGTAAATGGCCCGCCTGGCTGACCGCCCAACGACCCCCGCCCAT
TGACGTCAATAATGACGTATGTTCCCATAGTAACGCCAATAGGGA
CTTTCCATTGACGTCAATGGGTGGACTATTTACGGTAAACTGCCC
ACTTGGCAGTACATCAAGTGTATCATATGCCAAGTACGCCCCCTA
TTGACGTCAATGACGGTAAATGGCCCGCCTGGCATTATGCCCAGT
ACATGACCTTATGGGACTTTCCTACTTGGCAGTACATCTACGTAT
TAGTCATCGCTATTACCATGGGTCGAGGTGAGCCCCACGTTCTGC
TTCACTCTCCCCATCTCCCCCCCCTCCCCACCCCCAATTTTGTAT
TTATTTATTTTTTAATTATTTTGTGCAGCGATGGGGGGGGGGGGG
GGGGGGGCGCGCGCCAGGCGGGGCGGGGCGGGGCGAGGGGCGGGG
CGGGGCGAGGCGGAGAGGTGCGGCGGCAGCCAATCAGAGCGGCGC
GCTCCGAAAGTTTCCTTTTATGGCGAGGCGGCGGCGGCGGCGGCC
CTATAAAAAGCGAAGCGCGCGGCGGGCG
Exemplary CMV/CBA enhancer/promoter
(SEQ ID NO: 13)
GACATTGATTATTGACTAGTTATTAATAGTAATCAATTACGGGGT
CATTAGTTCATAGCCCATATATGGAGTTCCGCGTTACATAACTTA
CGGTAAATGGCCCGCCTGGCTGACCGCCCAACGACCCCCGCCCAT
TGACGTCAATAATGACGTATGTTCCCATAGTAACGCCAATAGGGA
CTTTCCATTGACGTCAATGGGTGGACTATTTACGGTAAACTGCCC
ACTTGGCAGTACATCAAGTGTATCATATGCCAAGTACGCCCCCTA
TTGACGTCAATGACGGTAAATGGCCCGCCTGGCATTATGCCCAGT
ACATGACCTTATGGGACTTTCCTACTTGGCAGTACATCTACGTAT
TAGTCATCGCTATTACCATGGGTCGAGGTGAGCCCCACGTTCTGC
TTCACTCTCCCCATCTCCCCCCCCTCCCCACCCCCAATTTTGTAT
TTATTTATTTTTTAATTATTTTGTGCAGCGATGGGGGCGGGGGGG
GGGGGGGGGCGCGCGCCAGGCGGGGGGGGGGGGGGCGAGGGGGGG
GGCGGGGCGAGGCGGAGAGGTGCGGCGGCAGCCAATCAGAGCGGC
GCGCTCCGAAAGTTTCCTTTTATGGCGAGGCGGCGGCGGCGGCGG
CCCTATAAAAAGCGAAGCGCGCGGCGGGCG
Exemplary CAG enhancer/promoter
(SEQ ID NO: 14)
GACATTGATTATTGACTAGTTATTAATAGTAATCAATTACGGGGT
CATTAGTTCATAGCCCATATATGGAGTTCCGCGTTACATAACTTA
CGGTAAATGGCCCGCCTGGCTGACCGCCCAACGACCCCCGCCCAT
TGACGTCAATAATGACGTATGTTCCCATAGTAACGCCAATAGGGA
CTTTCCATTGACGTCAATGGGTGGACTATTTACGGTAAACTGCCC
ACTTGGCAGTACATCAAGTGTATCATATGCCAAGTACGCCCCCTA
TTGACGTCAATGACGGTAAATGGCCCGCCTGGCATTATGCCCAGT
ACATGACCTTATGGGACTTTCCTACTTGGCAGTACATCTACGTAT
TAGTCATCGCTATTACCATGGGTCGAGGTGAGCCCCACGTTCTGC
TTCACTCTCCCCATCTCCCCCCCCTCCCCACCCCCAATTTTGTAT
TTATTTATTTTTTAATTATTTTGTGCAGCGATGGGGGCGGGGGGG
GGGGGGGCGCGCGCCAGGCGGGGCGGGGCGGGGCGAGGGGGGGGG
CGGGGCGAGGCGGAGAGGTGCGGCGGCAGCCAATCAGAGCGGCGC
GCTCCGAAAGTTTCCTTTTATGGCGAGGCGGCGGCGGCGGCGGCC
CTATAAAAAGCGAAGCGCGCGGCGGGCGGGAGTCGCTGCGTTGCC
TTCGCCCCGTGCCCCGCTCCGCGCCGCCTCGCGCCGCCCGCCCCG
GCTCTGACTGACCGCGTTACTCCCACAGGTGAGCGGGCGGGACGG
CCCTTCTCCTCCGGGCTGTAATTAGCGCTTGGTTTAATGACGGCT
CGTTTCTTTTCTGTGGCTGCGTGAAAGCCTTAAAGGGCTCCGGGA
GGGCCCTTTGTGCGGGGGGGAGCGGCTCGGGGGGTGCGTGCGTGT
GTGTGTGCGTGGGGAGCGCCGCGTGCGGCCCGCGCTGCCCGGCGG
CTGTGAGCGCTGCGGGCGCGGCGCGGGGCTTTGTGCGCTCCGCGT
GTGCGCGAGGGGAGCGCGGCCGGGGGCGGTGCCCCGCGGTGCGGG
GGGGCTGCGAGGGGAACAAAGGCTGCGTGCGGGGTGTGTGCGTGG
GGGGGTGAGCAGGGGGTGTGGGCGCGGCGGTCGGGCTGTAACCCC
CCCCTGCACCCCCCTCCCCGAGTTGCTGAGCACGGCCCGGCTTCG
GGTGCGGGGCTCCGTGCGGGGCGTGGCGCGGGGCTCGCCGTGCCG
GGGGGGGGGTGGCGGCAGGTGGGGGTGCCGGGGGGGGGGGGGCCG
CCTCGGGCCGGGGAGGGCTCGGGGGAGGGGCGCGGCGGCCCCCGG
AGCGCCGGCGGCTGTCGAGGCGCGGCGAGCCGCAGCCATTGCCTT
TTATGGTAATCGTGCGAGAGGGCGCAGGGACTTCCTTTGTCCCAA
ATCTGTGCGGAGCCGAAATCTGGGAGGCGCCGCCGCACCCCCTCT
AGCGGGCGCGGGGCGAAGCGGTGCGGCGCCGGCAGGAAGGAAATG
GGGGGGGAGGGCCTTCGTGCGTCGCCGCGCCGCCGTCCCCTTCTC
CCTCTCCAGCCTCGGGGCTGTCCGCGGGGGGACGGCTGCCTTCGG
GGGGGACGGGGCAGGGCGGGGTTCGGCTTCTGGCGTGTGACCGGC
GGCTCTAGAGCCTCTGCTAACCATGTTCATGCCTTCTTCTTTTTC
CTACAG
Exemplary CAG enhancer/promoter
(SEQ ID NO: 15)
GACATTGATTATTGACTAGTTATTAATAGTAATCAATTACGGGGT
CATTAGTTCATAGCCCATATATGGAGTTCCGCGTTACATAACTTA
CGGTAAATGGCCCGCCTGGCTGACCGCCCAACGACCCCCGCCCAT
TGACGTCAATAATGACGTATGTTCCCATAGTAACGCCAATAGGGA
CTTTCCATTGACGTCAATGGGTGGACTATTTACGGTAAACTGCCC
ACTTGGCAGTACATCAAGTGTATCATATGCCAAGTACGCCCCCTA
TTGACGTCAATGACGGTAAATGGCCCGCCTGGCATTATGCCCAGT
ACATGACCTTATGGGACTTTCCTACTTGGCAGTACATCTACGTAT
TAGTCATCGCTATTACCATGGGTCGAGGTGAGCCCCACGTTCTGC
TTCACTCTCCCCATCTCCCCCCCCTCCCCACCCCCAATTTTGTAT
TTATTTATTTTTTAATTATTTTGTGCAGCGATGGGGGGGGGGGGG
GGGGGGGGGGGCGCGCCAGGCGGGGCGGGGGGGGGCGAGGGGGGG
GGCGGGGCGAGGCGGAGAGGTGCGGCGGCAGCCAATCAGAGCGGC
GCGCTCCGAAAGTTTCCTTTTATGGCGAGGCGGCGGCGGCGGCGG
CCCTATAAAAAGCGAAGCGCGCGGCGGGCGGGAGTCGCTGCGTTG
CCTTCGCCCCGTGCCCCGCTCCGCGCCGCCTCGCGCCGCCCGCCC
CGGCTCTGACTGACCGCGTTACTCCCACAGGTGAGCGGGCGGGAC
GGCCCTTCTCCTCCGGGCTGTAATTAGCGCTTGGTTTAATGACGG
CTCGTTTCTTTTCTGTGGCTGCGTGAAAGCCTTAAAGGGCTCCGG
GAGGGCCCTTTGTGCGGGGGGGAGCGGCTCGGGGGGTGCGTGCGT
GTGTGTGTGCGTGGGGAGCGCCGCGTGCGGCCCGCGCTGCCCGGC
GGCTGTGAGCGCTGCGGGCGCGGCGCGGGGCTTTGTGCGCTCCGC
GTGTGCGCGAGGGGAGCGCGGCCGGGGGCGGTGCCCCGCGGTGCG
GGGGGGCTGCGAGGGGAACAAAGGCTGCGTGCGGGGTGTGTGCGT
GGGGGGGTGAGCAGGGGGTGTGGGCGCGGCGGTCGGGCTGTAACC
CCCCCCTGCACCCCCCTCCCCGAGTTGCTGAGCACGGCCCGGCTT
CGGGTGCGGGGCTCCGTGCGGGGCGTGGCGCGGGGCTCGCCGTGC
CGGGCGGGGGGTGGCGGCAGGTGGGGGTGCCGGGGGGGGGGGGGC
CGCCTCGGGCCGGGGAGGGCTCGGGGGAGGGGCGCGGCGGCCCCC
GGAGCGCCGGCGGCTGTCGAGGCGCGGCGAGCCGCAGCCATTGCC
TTTTATGGTAATCGTGCGAGAGGGCGCAGGGACTTCCTTTGTCCC
AAATCTGTGCGGAGCCGAAATCTGGGAGGCGCCGCCGCACCCCCT
CTAGCGGGCGCGGGGCGAAGCGGTGCGGCGCCGGCAGGAAGGAAA
TGGGGGGGGAGGGCCTTCGTGCGTCGCCGCGCCGCCGTCCCCTTC
TCCCTCTCCAGCCTCGGGGCTGTCCGCGGGGGGACGGCTGCCTTC
GGGGGGGACGGGGCAGGGCGGGGTTCGGCTTCTGGCGTGTGACCG
GCGGCTCTAGAGCCTCTGCTAACCATGTTCATGCCTTCTTCTTTT
TCCTACAG

In some aspects, the promoter is a GJB2 minimal promoter as set forth in SEQ ID NO: 86. In some aspects, a promoter is at least 85%, at least 90%, at least 95%, at least 96%, at least 97%, at least 98%, at least 99%, or 100% identical to SEQ ID NO: 86.

Exemplary Human GJB2 minimal promoter
(SEQ ID NO: 86)
AAGCTCTGAGGACCCAGAGGCCGGGCGCGCTCCGCCCGCGGCGCC
GCCCCCTCCGTAACTTTCCCAGTCTCCGAGGGAAGAGGCGGGGTG
TGGGGTGCGGTTAAAAGGCGCCACGGCGGGAGACAGGT

In certain aspects, the promoter is a GDF6 promoter as set forth in SEQ ID NO: 90. In some aspects, an promoter sequence is at least 85%, at least 90%, at least 95%, at least 96%, at least 97%, at least 98%, at least 99%, or 100% identical to a promoter sequence represented by SEQ ID NO: 90. In some aspects, the promoter is a GDF6 promoter sequence comprising the sequence of SEQ ID NO: 90.

Exemplary Human GDF6 promoter
(SEQ ID NO: 90)
CCACAGGTAACTCCGTCGGCGTCCACAGGGGGGCAGGAGATACCA
TACTGCACAGTTGTACGTCTTCCATCTGTTTGGTGTAGAAAAATC
TAACCACTACAAGAATGCCACGGGCACTGTGGCAGACAGAAGCAG
CGCTACGCCGCATCGCCTTTCAGCGTGCAGGCCCAGGAATGAGCG
AGGCAGTGGGCGGGGAAGACAGGCACGGGGAATCTGGGGACAGAT
AAAGGAAACTCGTGATGGGGCGAGGCTGGGCTGAAGAGAAACAGA
TTGGGGTAGAGCTGCAAAGGGAGGGGTCCACTGGAAGGCGAGGGG
GGAGGCCGGGAAGAGAGAGGGTGGGAAGGCAGTGTGAGATGGGAG
GGCAGTGTGAGAAGAAAAGCAGGCTGGGGAAGAGGGATTGGAATG
CAGAAGGAACTTGGGGAAGGAGGAAGTCCTGCAGGCGGGAGGGAA
AGAAGAGAGGGGGAGCAGCTAAAGTCTGCGTCAGAAGAGGTTGGG
GACTGCGAGAGGAGAGGCTGGGGCCTGCAGGGGAGCGCAGCAGCT
TTTAGCATCGATCCAAACTCTAAAGACTCGTGGCCTTTGCCTGAC
CTCGAGGGTCGGGAATAGACGCCTGTCTTTGTGGAGAGCGATACC
CAACCGAGAAAATGGGGCTGTTCCGAGCTGGGCCCTGCGCCTGGC
CCAGGGCGAGGCTTCTCTGGCTCCGGGCTGGCCCCTGAGGGGCAG
CACGCAGCCTGCAGCAGAGGCGCCTGCTCCAAGCTGTCTCTTGGG
GGCGCCGCCGCCGCTTCCCTCCTCCGGGGCCGCTCGCTCCCAGGA
AAGTGGAGGCGGCTGGCGAGGACCGAGAGCCGGGGCCGCGCTGCG
GAGGGACCACACCTCCGGGAGTTCGAGGGGGACCCTGGCGCGGCG
GGCCAGCCTTTCGGGCCGGCAGCGCCCGCCTTCCCCCGGTCAGCG
CTTGCGGCCCGCGCCGCGCGCACCGCCCGGCAACCCCGCGCGCGT
CCCGCGGGGGCGCTGCGTCTTCCTGCCACACCGGCGCACCGCGGC
CCCTCTCCCCCACACCTCCGGCCCGCACCACCCGGCTCTCCTCCC
ACCCTCCCCACCCCTCCTCTGCCCTCCCTCCCCATTCCTCCCCTC
CCGGCGAGGGGGGGGAGGGGGCGTGGCGGGGCCGGGGTTTGTGTG
GCTGGGACCCGGCTCCTC

In certain aspects, the promoter is a human IGFBP2 promoter as set forth in SEQ ID NO: 57. In some aspects, an promoter sequence is at least 85%, at least 90%, at least 95%, at least 96%, at least 97%, at least 98%, at least 99%, or 100% identical to a promoter sequence represented by SEQ ID NO: 57. In some aspects, the promoter is a human IGFBP2 promoter sequence comprised within SEQ ID NO: 57.

Exemplary Human IGFBP2 promoter
(SEQ ID NO: 57)
AAGAAACTTGCCCGAGTTTACACAGCTAGTAAATGGTTGCATTAG
TCAGGACAGCTAGCCTATATTACAATAACAACCCTCTCAAATCCT
AATGGCTTAAAACAACAGAGGTTTAATTTATACTCATTAGCTGTT
CAAGGCAGGAGGCTCTATTCTCTAATCCATACAGTCACTCAGGAT
CCAGGCTGGTGGAGACCCTGCCATATTGTAGCCTCACCATTTAAA
ACATGAAGAAGATAGAAAGTGAGGAGTCATGTAGGTTTTGTTCCG
TTGCCTCAGGCTAGGAGTGACAGGTCACTTCATCTCACTCACAGC
TCACTGCCCACAACTAGTCACTTGTGACTGTGCGAGTTAAGCTTC
TGTGTGTGAAGGAAGGAAAAGAGAATGGGATAAAGGTGAACATCA
GCAGGCTCTACCACAGTAGTTTGAACCAAGACTTGAGCCTAGGTC
ATGTGGCTTCAGAATCTTTGCTCTTAATCACACTAAACAGCCTCT
GTAAGTCATCTTTCCTTCATCCAGTGCCTAAGAACATGCAGTCCA
ATGCCCTCATCCTTCAGAAGAACTTGAGTGAACTCAGAGAAATTG
AGTAGAGTGCCACAGCATGCCCAAGGCCACACACCCTGAGGTTGG
CAGTAGGTCCTGAGTTAGAGTTGTCATTTCTTGGCTCCCCTGGTA
GTAGTGGAAAGGTAAGGTTTTGACATACTAGTTGGATGACCACGG
GCAGGTCACTTAAATTGTCTAAGCATCGTTTGACCCTTGTAAGAA
TTAAATGAAATAGCACCTGTAAAAGTGTCTGCACGGACTTACTGC
TGTTAGTTTTGTTCCTTTCTTCCTGTTGTCACTGCACTTCCCTGC
CTGTTACCCAGGCCATGCAGACCAGCCAGGCCTTCGACTTACAGT
GCGGATAAGATTCCAAATCTCCACGGCTGGTTTCCATGCTTTCTT
CCAGGCTTCTGAGGACCCTGTGCTCTGGTTTCTTCTATTTCTTTT
CTATTACTTTTCTGTTACTCTTGAGCACACTTGCTGGAAGCAATA
TGCATCCAGTTCTCCCTCTCTTGCCTCATTACACTTTGCAGAACA
ACTCCAATCCCTTCCAACCAAGTAGTCCCTTTGAATTTCTTGTCA
CCCAAGGAATCTCTCTGACAGGGGTCTTTGTTAGGGTCACACCCC
AGGAGATGGTTGATTATGGCTGAGTCCAGCCTGGAATGATGGGGG
TTGGGGGCAGCTTGGGTAGATGACTCAGTAAATCAAACAGAACAA
TGAAAGGAGGTCATGCTTGTCCATCTGCATTATTGAAGACAGCCA
TAAATGGCCTTACCCCAGAGCGGGTCTGTCACACCTGGAGAGCTG
ATCTGACCTCTCCAAGACCCCTGCAACTGAGTGTTCTGGGATCTG
TCCTGCAACAAGTGCCTCGAGATTTGTAGGTGGGGGCCCAGAGGG
AGGGGGTCTGCAGACGAAGGGGGCAGGTTTTGCGGGGCACTTAGG
GTTCTCATAGGTTGTAGTCACGAGCTCC

In certain aspects, the promoter is a human RBP7 promoter as set forth in SEQ ID NO: 28. In some aspects, an promoter sequence is at least 85%, at least 90%, at least 95%, at least 96%, at least 97%, at least 98% at least 99%, or 100% identical to a promoter sequence represented by SEQ ID NO: 28. In some aspects, the promoter is a human RBP7 promoter sequence comprised within SEQ ID NO: 28.

Exemplary Human RBP7 promoter
(SEQ ID NO: 28)
CCCATGGCTCTGTTAAAATCAAAGAAACATCTTTTCCAACAGCCC
TTTCAAACTCCTCATCGCATCTCACTGGCTGATTCAGTCATTTAA
ACCTGCTTCTCCCTAAAGCTGATCACTGGCTAAGCTAATAGGGTT
TCCGGGATTGGTTTAGCCTGATACTAATCCAGGTCTACCTTCAGG
AGCCAGACCAAACTGCCTATTGGCATTGCATTCTTGCAGTAGGGA
GGGGAGGTATGGATGGTGTGGAGTCCACCACAAGGTCCATGCCAG
TCTTTGCTGAACCAGCATCAGACTCCATCAAGCAACAGATGAGAG
GTTCCATGATAAAGTGGCCCTCAGCAATCCCCATCCATTGCTGTC
TAGGAAGAACAGTGCTTGTACACAGGTTTAGGACCTCAGTCTTGG
CTGTAATCTTCTGGTTTACTTTGCCAGCACCAAACAGAAGGAAAG
AAAGGGCTCAAATTTGACCAAATAAATTATGCTTCTCCTTCCAGA
GATAACCTTGAGTCCTGTCTAGGAAGATATTAGAATTGTAAAGAA
AAAAAAAATTACTCCTTATCCTATGGCAAGTGGAGTCTATGTCTA
CTTCAGCTGAAATTAAATCCTGTCCATAATAGATGACCCTTGCTC
AAGCTGGCCAGAAGCCATACCAACCAGCACGAAGGTTAAAACTAT
TATTAGTTTTTTCTGTGATTTTCATTTTCAGGCCAAGTTTTAGAA
CAATAAGATTTTAAGAATAGGAAGTAAGTAAGATTTCTGCATATC
CTGTTCTCTTAGTCAGCTGAATTTTTTTTTTTTTTTTTTTAGTCC
TAACTCAGCCTCCCAAAGTGCTGGGATTACAGGCGTGAGCCACCG
CACCAAGCCTGGAATCTATGTCTTACAGTTATGAGAATCAACAGC
TAGCTCATTATGGGCAAGGTGATGTCACTCTGGCTTCTCAATGAA
AATGGCATTTCTCCCTTGGAAAAGGTCATAGCCAGTCAGTCAGTC
AGTCACGGGAGCGCAGCGGCTTCTAGGGGTGAGTGGGACCCACGC
GGCCCCACCTGCTCCTCCCGCGCGCGGCCCCACCCCCCTGCCCCG
CCCCGCCTGGTTTATAG

In certain aspects, the promoter is a human GJB6 promoter as set forth in SEQ ID NO: 16. In some aspects, an promoter sequence is at least 85%, at least 90%, at least 95%, at least 96%, at least 97%, at least 98% at least 99%, or 100% identical to promoter sequence represented by SEQ ID NO: 16. In some aspects, the promoter is a human GJB6 promoter sequence comprised within SEQ ID NO: 16.

Exemplary Human GJB6 promoter
(SEQ ID NO: 16)
AAATAGCTTCCAACGTTTCCACCCCACCAGCCCTTGCACCACTCC
CTGTACTGGCCCTGAGCTTTCTAGTCTTGACTGAAAAGCGGGGAG
GCAATGTGGTCTCTCCTGGTGCACTGTCCCGAGGAAGGCCTGCTC
CGCTTCCCCGGAGGAGTCTTCAAAGGATGGAGGTAATTAATAAAA
ACAACCCCTGTACCTCCTCTAAGTGGTCATTAATTAATAAAGAAC
CTCCAGGCTCCTATAGGAGAGGTCTGTGCACCCCGCGGGCTATGA
GAAGGCTGGATCACCCAGAAAGACTGAGGATGTGTCCTGGCAAAA
ACACAGCCTGCCCCTCACACTGCTCCCCACGGGTGCACTAGGGAG
GAAGAGTTCCCTCGAGGGCCTGAGCAGGCGCCCCACACCTGCACC
CGTGCAGAGGGGGCTGGGCCCGCCCTCTGCGCTCCCGAGGGAGAG
CCCTACCCCCTGCATCCCCGGTACCCCGTTCCCTCCAAGGGCCGG
AAAGAGGGCCCCGCGCACTGTGCACTTCTTAGGGGTCCCCCACCC
TGCGCCCCCGCCACGGGAAAAAGGTCCCCGCTCTGCGCATCCGGC
CCCGGAGGGACAGCCCCGGTCCTGCACTCCTTGCTCCTCAGGGGG
ACGGTCCGCGCCCAGCGGCTAGTGCGCCCCGGGTAGGTGGGGGGG
GGGGGCTCGTCGAGTGACAGCGCTCGCCTCCCGCAGCCCGCCCGA
GCCGCGTCAGGGCAG

In certain aspects, the promoter is a human PARM1 promoter as set forth in SEQ ID NO: 40. In some aspects, a promoter sequence is at least 85%, at least 90%, at least 95%, at least 96%, at least 97%, at least 98% at least 99%, or 100% identical to promoter sequence represented by SEQ ID NO: 40. In some aspects, the promoter is a human PARM1 promoter sequence comprised within SEQ ID NO: 40.

Exemplary Human PARMI promoter
(SEQ ID NO: 40)
TGTACAGGAGATAGTCAGGGAATTAGTAATTTTCAAAGAGGTGAC
TTTGAATTCAAACTTAAATATCATCTTCAGCTGAAACAAAGAAGG
GGTGCAGTTATGAGGAAGTGACCAGGTAAAGCATGGCAAACAAAG
GTAAAGTTTGTTATGCGTATTTAAGTCAGAGCCCTCTCCATTGAT
AAGAGTTTCCAGTAATTTAGTGCCATCCTTTTCTTGCTATAGAGT
TCTCGTCTCTATCTGAGCACGCAAAAATAACATGCTTTCTTGCTT
TCTTGAAGTTGGGCATGGCCATTGACTTGCCTTAGCCCATATTTT
TCTGTGAAGTGGTCTTCAAAAACCTATATTTCTGCCATAGAGTCA
CTTACTTAACCTGCCCTATTTAAAGGGGCTAATGCCTGATAGAAT
GTCGCTGCATAACTCCATCTGTGTGTGGTCCCTGCATCCATGACA
ACCAAAACCCAGATGCAGAAATTGTTCCTAATCACATAGATTACC
CTAGAAACCGGAAGGGCCTTGAAGTCAAAAGCATTCAGAGAACAT
GCTGAACAAATTGAATTTGCAGTTTATCTGGCCAGGGAGGATGGA
GAGGGGATGGGCACTTGGTCTGAGTATTTTTTGTTTCTCATTCCA
ACAGAAATTACTAGATTTACCAAAAAATCTACAAGTGGTAGTGTG
ATAGAGTCAGGCAGAGGAATTGACCATAGATAAGGTGCTCAGGAC
TCCTAGAGTCAGCTTCTGGTATGTGAGAAAGAAGTGAGAACAGAG
CCCATGGCATATGAAGAAGATATTACAGAAAAAAGAAAGCTGCCT
TCCACGCAAATCATTTCTTTACAAAGGCTTGTTAACTCCTGCAGT
GCCAAGAAGCTGAATGCAGCGGCAGACATCCTGGTTCGGGCCCCA
GGAAGCTCAGCCGGGTTTAATGTGGATGAGGGTTTAATGATGTAC
ACGCAGAAGTGTTTTGACAAATGAAGAAGGTCCTCATTCTTGGAA
CATGTGCCGGTTCTCCGAGGGAACTCCTAAAAGGCTGTAAGCTCA
TGTAGGAAAAGCTGAGCTAGATTCCTAAGGGCAGAGATGTGCTCA
CATTTCTTTGCATCCCTAGTTCCCAGCACAGTGCAAGGCGCTGCA
AACATTTGCTGAACCCAGGGTCTCGTGTCTTGACTGTCCAGCAGA
GGCCGCTCTGGGCCGGGGCTCTCGGGACCTGAGGGCTGAGAGAAG
GAAGGCCAGGGGGTGGCCCAGTCATCGCCGCGGGGCCCGGGTGGG
AGGGGTTTGGCAGCGGCAGGCGCGGCGGCGGCGGCGGAGGCGGAG
GCGGCCCCGGG

In some aspects, the inner ear supporting cell selective promoter is a BACE2 promoter. In some aspects, the BACE2 promoter comprises a nucleic acid sequence with at least 85%, at least 90%, at least 95%, at least 96%, at least 97%, at least 98% at least 99%, or 100% identity to SEQ ID NO: 92. In some aspects, the BACE2 promoter comprises the nucleic acid sequence of SEQ ID NO: 92.

Exemplary BACE2 promoter
(SEQ ID NO: 92)
TGTGCTGCGAGGGCTTCATCTCCTAAGCACTAAATGCTAAATTCC
CCCTCCCACGCCCATCGCCACTGTCCTCACGGATCCTCGCAGCAG
CTTCCCAATCGGTCTCCCTGTCTCCAGCCTCACCACCCCCAACTA
AGACCATTCATGAAAACAGAGACAACCAAGGAGACAGTCACCCAA
TGCTGTCCCTTCAGCTTGCATTATTTTCTGACAAGACAGCTCTGC
CATCCATGGAAGCCTGTGTTTGAAGATCTCTGACATAAAGGTCCC
TTGCAGAGCTAGACGTGATTCTAAAATTGGGAACACAGGAATAAA
AATCAAATCTTGAGTAGAAGTAGCTGAAAATTGCAGTGATTCGGG
GAAGCTTGGCTTCTAACTCCCCACTGTTTGAAGATGGGCTTGTTT
GTTTTTTAAAACAGCCAACATAATTCAGCTGGAGGAGGTACAAAG
AATTTTCTATTCCTTGTTTCTGTAGAAATCGATGGACTTTAGCTT
GTCTAATTGTCCCCCCTGCCTTTAGTATCTAAAATAAAATAACCC
TCGTTGCTTGCATTACTCAACGCATTTCTGCGTCTTGGCGTCTAT
GGCTAAACGAGTATTAATTAGACAGTCCGCAGAGAGCTGGCTGGG
GATAGAAGGGGAGGTGGGGGAGAAGGGCAGGGATCACAGCAGGGT
GGACTCGTGGCCCTGATTTGGGATCCTGACAGCAACTTACTAGGT
GGCCTGAGGGCTGGGTGCCAGGGGAGGCAGCGGGTTCCAGTAGCA
TCTGACCTGCATTAGGGACAGGGGCGCGGCGGAGGGGGCGAAGGG
GGCGGGGGTGGGGGGAAGGTGGCTGGGGTGAAGCCCAGCTTCGCA
GCTAGCTGTGGGCAACAGAGGGAGTAAGGGGGGGCAATGAGGCTG
GGGCCAGGCGCCAGCAGCAGCCACGCCCCCCACCTCCCCCGATTT
TTAGGGAAAATTCTCCAAAGCTCTCGCATCCTCCTCTGCCTCCTT
CCACCCTCCACCCTCCCAGCCTCCACTGAGACCTCTTTAAAACCA
CCCAGGGGCCGCCGGGGGATGAGGCCGGGGAACGGGCTGGACTGA
GGGCGGGGGCTCGGGGGCAGCGGACGGGAAACGCCTCGAAAGCAG
CCAGACCCGGCGACTGAAATGAGGCGGAGGAGCTTGGCGAGGGGA
GGCGCAGGCTCGGAAAGGCGCGCGAGGCTCCAGGCTCCTTCCCGA
TCCACCGCTCTCCTCGCTGACCTCCGAGTCACCCCCGGAAGCTCC
CGCCACTGCCGGGCGAATAGACCCCCGCGGACCCCCAAGCGCGCG
GGGCCGGGGCCCTAGTTCAGGCCCTCGCTGCCCCTTTAAGGGTTC
TCGAAACTTTCCCCCCGGTATCAGATGAGCCTCGTCACATCCGTT
GGCCGTGGC

In some aspects, the inner ear supporting cell selective promoter is a DBI2 promoter. In some aspects, the DBI2 promoter comprises a nucleic acid sequence with at least 85%, at least 90%, at least 95%, at least 96%, at least 97%, at least 98% at least 99%, or 100% identity to SEQ ID NO: 93. In some aspects, the DBI2 promoter comprises the nucleic acid sequence of SEQ ID NO: 93.

Exemplary DBI2 promoter
(SEQ ID NO: 93)
GAAGAAACCTGCATTTCTTACACTTCAGTGTACTTTCCCCATATT
TAACTCCAAGATTTTTGTTAATTTGTTTGGTTTTCCTTTCTCAAA
CAAAATTATGCTCAGACTGAAAACCCTAGATTTGTTCCCTATTGC
ATCTTCATTTCTTCCCAAACATTCCATAAAACGTGACCTACATTA
AGTTAGCAAGTTAAGTCTGAAAGCGTCTACCTTCCCTGGGGAGGG
GGAAGGTGTAGGCAGGGCAGAGATTTGTAGTCCAGCCCTCTTGCC
ACAAATTATGAATTAGAGAGGAATGACTTTGCTTTTTTAATGATC
TCCAGAGAATTTTCCATCATTTCCCTCTCTTCACCCAGCTCCTTT
GCAACCACTGCCAGAGAAGTCTTCCTTTAGCTTCTTAAACATCGA
TCCTAAAACACTTCCAGACACCTGTGCTGCTCCTTTCAGTTCCCA
TGGAGATTAGGCTGTGTAACAATCTCGCAAAGACGTTCCCCTCCG
TCTCCTCATCCTCTTTTCAAACCCTTTTACGATTTCCCATCTCAC
TCAGCATGACAGTCAAAGTCCCTGTGATGGCCAACTTCTGCATCA
CCTAGCCAGTCTGCCACCGCCAAAACTCTCCAGCCTCATCTTACA
CTTGTTCTCTGCTTGGAATCTTCCCTCCCCTCCTTGAGGAACTTT
CTCAAATGTCACCTTCCCTCAATACTCCCCCTCCTCCATTTAAAA
CTATAAACTTCCAACTCTCTAAGCCCCTAAAGTACTCTATATTTA
ACTTATTGTATAAACTACTGTCCCTACTTGTAAGTTCCAAGATTG
CAGGGATTCACCCGCTTTGTTCACTGCTGTCTGCCAAGGTCTAGA
ACAGTGCAAGTTACCCAACAGGAGTTCAATAAACAGCCATTCATT
TAACAAATATTTGCTGAGCACTTCGTCCCGTCCAAGTTTGTTAAA
TCAAGACAAATAAGACACCGTCCCTGCCTTTAACGCACCAGATGG
AGAAATGCACCACAGACATAAATGTGCAATACAGGCCTGACACTA
CGGCCACAAGCAAGTCAAAGAACGTGCCAAAAGTTCAGAGGAAGA
AGCCTCGGCTTCGCCTTTCGGGAGACCAGTCCAGCTTTCCACCAT
CACGCTGCTCATCAGGGACCATCTCCGGGGGTCTCCTCTAGACCC
CAAGGGAGGAGCGGGTCCCGCCCGCCATTCCCAGGTCTCAGAGTT
TACTTGTCCAGAGATGCAACTTCCGGCCTCTTCAGGCCGGGCAAG
ATTTAAGGAAAGAAAAGAAACATAAGGACCTCCGTTCTTCGGTCT
CCGTCCCCTCCCCTTCCCCCGCGTGCCCCACCTGTTCCCGGCGTC
CCCTTCGGCTACTCCCGGCGTTTGCGCAAGCGGTCCCACGTGGGC
TCGGGCGGGGCTAGCGCCGCGGCGGGGGCTGGGCACGCCCCTAGC
GCATAGCTGGCTTCTGATTGGCTTTCC

In some aspects, the inner ear supporting cell selective promoter is a FABP3 promoter. In some aspects, the FABP3 promoter comprises a nucleic acid sequence with at least 85%, at least 90%, at least 95%, at least 96%, at least 97%, at least 98% at least 99%, or 100% identity to SEQ ID NO: 94. In some aspects, the FABP3 promoter comprises the nucleic acid sequence of SEQ ID NO: 94.

Exemplary FABP3 promoter
(SEQ ID NO: 94)
TACCATTCTGCCTTTCACCTGATGTTGCTATCCTCCTCCCTCTTG
TTTCCTTCCACCCATCCTTTCCCTCCCACATTACTCTCTTATCCC
ACCCTATTTTACAACCAGTAGCCTAGGGAAAAGAGCATAGCTCAA
ATGAGGAAGAAGGCAGGACAGGCAGTCATGGCTTAGCTGGACTGA
GCTGCAGTGCTTCTCCTTCTGGGGAAGGGGGTGCACTGTCATCTG
CTACTGACACATCCCTCCAAGGCACTCAGCCCTGCAGGGAGCAAC
CTGATTCTATGACTGACATCTAATCTTCACATTCACCTTGCAGGA
AGGCAAGAAGTGATCCCAGCCTCCAGATGGAAAGATCAAGGCCCA
GAGAAGGTCAGTGGTGGTTGGAGGCCTGAGGTCACACAGCAGCCA
AGTCTGGAGTCACTAGTCAAGGTGACCTTGACTAGCCACCCCACC
TCCCCTTCCCTGCCCCACCATGGCCCTGGGAGATCTGTTGTCCTG
TGAGGGAAAGGGGCTCCAGGCTGGGCTGCATCTGAAGCCCCTAGA
TCCAGAGACTTCATTTCTTAGGCTATCTATAAAATCCACCTTCCT
TTCTTTTCCCAGGACCCCCATACCCTGCTCCCAGCATCGTCTGCC
TCAGCTAAGCCATGGGGATTGAGAGACCAGGCCTGGTGCCCAGAT
AAACTGACCCTGGGTGAGGGGACAGGGGCCCAGAATGGGCAGGTA
GAGACTGAATACTGAAGAAGAATCCTCTGGAGTCTGTTAGCAGAA
GCAGATGGGCCTTGCCTGACTATTGGCAGGCGGACCTGGTGGTCA
GACCTCAGTGATCCTCAGGGACCAGTGAATATTTCAGGCTGGGGC
TGAGCATCACCTGCTCCCTTGGCCCCACTTATAGGGCAAAGGGGA
GTCTACCAGCCTACTCACTGATGACAAACTGGAAAAGTTTGTCCT
GTCTCTGCTCTGGCCCCACCTCGCCCTCTCCCCTACTTGGAAGTT
CCTTTCCTGAACCACTGACTGCCAAAGCTTGAGGGATTAAATAAA
TCATCTGGCCCAACCTCCTACCATAGAGTTGGGAACACTGAAGAA
AAGAGACTGGCCCAAGGTCACAGAGAAGGCAGGGTGAACACTGTC
ACAGGGAGAGCCAGTGTAGAATAATGGTTAAGCCACGCAAGCTCT
AGAACCACTCTATCTGAGTGCAAATCCTGGCTGTCATCTGGTACT
TGCTTCCTGGAACACATCTGGCCTCAGACTCCTGAGGCCAAGACA
CACTCCCTGCCCTAAGACTTGCTGGTTCTATGGCAGGCAGAGGCA
GAAAGAGCCCCACCATCATTCCCAGCAAATGGGAAAAGTTCCCAG
TTGCAGATATTAGGGGTGGGATGGGGCGGGGGTAGTCAGCAACCA
TAGACTTAGACCCTGAAGAGGCAAAAAAGGAGGGCCATGTTCTTG
GGTCAGCAGAGCTTCTACTCAGCTTCTTCAGCCTCTAGCTCTTTC
CTGGTGCTAGTAGCACATTCTCTAGTGGAGGCATCCAGATGGCAG
GGAGGGTCCAGGAAACAGCTGAACATGCTGAGCAGGCCTCCCTTG
TCCCCGCTCCCCATGGCCCCATGGATCATCCGGTGCTGCAGCTCA
TCTCATTGGCTGGCTTCTGGTTACTCATCTCTCCTCTTCTCCATC
TTCCCAGCCTGTGGTTGCCGTGGAAACATAGAACAGTGACCTCAC
CATAGGATGAGGGCTGGGGAGATGCTGTTCTTGGCAGGCGCT

In some aspects, the inner ear supporting cell selective promoter is a KLHL14 promoter. In some aspects, the KLHL14 promoter comprises a nucleic acid sequence with at least 85%, at least 90%, at least 95%, at least 96%, at least 97%, at least 98% at least 99%, or 100% identity to SEQ ID NO: 95. In some aspects, the KLHL14 promoter comprises the nucleic acid sequence of SEQ ID NO: 95.

Exemplary KLHL14 promoter
(SEQ ID NO: 95)
GAAACAGCAGCCATTGATGTAGCTCAGGGTTCTGTGGATCTGTCA
TTTGGAGCATGTTGGTTCTCCTGTCTCAGCTGGGCTCATTCATGC
ATCTGAGTTCAGCTATTGGGCAATCTGGGGAATGTTTTGTCCATG
TGATGTGTCATCTTCTACCAGGCTAGCCTGGGCTTCATCACATGG
TATCTGGCAGGGCTCTAAGAGGGAGAGTTGAAACACACAAGGCCT
CTTGAAGCTTAGACTCAGAATTGGCACAAGGTCGCTTCTGGCACA
TTCCATTGGTCAAAGCAAGTTACAAGGCCAGCTCACATTCAAGGA
TTAGGTAAGTCGATTCCACTCTTGATGAGAAGTCTGAAGGATTTG
GAACAGTGTCCACCATGCAGTAATAAACTCAATAAGTAGTAGCCA
TTATTATTCTGTTAGAGGTTGCCAGGAAAAGTTTTATAGTGGAAA
GAAATCTGAGTTTACTCTTGAGAGGTAAGTGGAATTTCTATTTGT
AGAGAATGAAGGCCTCTCAAAAAGACACAGCCTAACAATAGGTGC
TGCAGTTTAACAGTGGAGCGTGTCCAGAACAGGCTGCCCTTTTAG
GCAAGGGCTAGTGTCTTTCAGGACAGACCCAAACCCCAAATACCA
AAACAGAATAAAGTAGTGTCTTAGCATACTTTGAGATCAGACTGT
TTCTGCATTTCACAGTGCTGGGGGTGGGGGGGAGGTGTGGGGGGA
AGGGAAAAGCAGCATACCAATGTAGTGAAATCTGGAAACAACAGC
CAAAAAAAGTTTGCATATTGCACAGAGCACTTGAAGATCATAAAT
CTATGCATGAGAAAGATGTAGTGGAAATTTTGGGGGGGATTAGAG
TTTATTTTTGTCATCTCTGTGAGACAGCTACTCATTCATCCAGAT
CACAGCTAAGAAAAAAGCTGGTCACAGAAATTAGCAGTTTCAGCT
CAGCAGCGAAGTCGCCAGCCTGTGAAGGCAGAGAGAAATTGACTA
ATTAGCAATGCGCACTAAAACTTGACGGTTCTTTATAGAGAGAGA
GAAGAGAGAGGGAGAGAGAGGGAGAGGGAGGGAGGGGGGGCTCGC
TTTTTCCCCTTCTTTCTTCCAAAGATGTTTGAAATCGCAGTCATT
TACGCTCGACAATTTTTACAATAGCCTTGAGCCATAATTTTGCGA
GTCTCTCCAGCATCCATCCCCCTGTATGGTCTCTCTCTACTGGCC
AAGCACGACCGTTTCTCTCCCCAACCGTGGATTTCCTATT

In some aspects, the inner ear supporting cell selective promoter is a MMP15 promoter. In some aspects, the MMP15 promoter comprises a nucleic acid sequence with at least 85%, at least 90%, at least 95%, at least 96%, at least 97%, at least 98% at least 99%, or 100% identity to SEQ ID NO: 96. In some aspects, the MMP15 promoter comprises the nucleic acid sequence of SEQ ID NO: 96.

Exemplary MMP15 promoter
(SEQ ID NO: 96)
CCTTCCTCCTCCAGGGCCCTCTGCAGACCAGGCTGAGATGGAGGA
ACCTGCTAAAATCGATGGAGATGCTTCTAGCCTCCCAGTAGGAGG
CCCCAGCCATGCCTTCAACCTGGCAGGAGGTGTAGCCACTCCTCA
TCCTTGGGTTGCAGGTTGGGTGCTGCTGTTGTGGTCCTTCCCAGA
AACTGCCAGTAGAGGGCAGCCTGGGCATCCTAATGCTTACTCTGG
TTGTTACACAAAGAAAATATTGGGGTCACTGGCGAGCCCACCCAC
ACTCACCAGAATCTCCACTGTAGTCCCCCTAACAAACAGCCCTTC
ACTTCCTCTCCCACTTCAGCAATTTGTATTTTGATGCCATTGGCC
TCAGATCAGAGTGTTTTAAATCATCACGCCCTGGCTTATCCCTGG
TCGAGCCAGGACACGGGGTGCTTCAGTGGGTCTGTCACCCTCTCT
CCTTGAAGCATGTTGCTTTTATTTATTTACTTTTACTCTCACCCT
GCTCCTGTACCAGCAGGGGCCACTTCAAAGCCAAGGTACAGGGTG
ATAACTTGTGGTCCAGCATCAGTTTTCTCCACTTCTTTCTCCCAC
TCACCCCCAGCAAGGTGCCTGGGGAGACTTGAGCAGATGTTTCAT
TTTGGCCTGGCCAGTGGCTGAAAGCCAGGCCTCCAATGCACTGTG
ACCTCTGGCTTCCCCAGCAGCTTTCCCAGAGAGGCAGAGGGAGTC
TTCATTCTTCCCAGGCGGGGAGACCACGCCTTCCCTGCCTCCTCC
CTCCGCGGGGGGTCGCGTTGGAGGTCACCCCCGCCCCCTAGGCGC
TGGGTTGGGAGTGACGCGGGGTGGGCTGGAGAGGTTTCCTGCCGT
CTGGGAAGCGTAAACGGACCGCCCACCTGTCGGGCCTCGGCCGCC
CGCACCTGCTTGTGAGAAGCCTGCGGCTGGGGCACCGCCCCCGGT
CCCCGCCCGGGTCCGCGCATTGGGAGCACACTGGCCCTTTAAGAG
CGCGGCGGCCGCGGCGCGCGGG

In some aspects, the inner ear supporting cell selective promoter is a SPARC promoter. In some aspects, the SPARC promoter comprises a nucleic acid sequence with at least 85%, at least 90%, at least 95%, at least 96%, at least 97%, at least 98% at least 99%, or 100% identity to SEQ ID NO: 97. In some aspects, the SPARC promoter comprises the nucleic acid sequence of SEQ ID NO: 97.

Exemplary SPARC promoter
(SEQ ID NO: 97)
CAGGCTACCTCTCAGGCTGACTGAGTCATGCAGCATAGGCTGCCA
CGTCTCTGGGCTGGCGGGGCCGTCATTATTCCTGGCCTCACTGCA
GCTAAATTGAAGAAACGTTTGGTTTGTGGGCCACGTCAAGGAATG
TGTAAGAGCTGCCACGTTGTCGGGTCTGGGTTATTGGGCTTTTCC
CCTCCTTCAGAGAAGATTTCCAGGCGTGTGGGTGGGGTTTCAGAA
GAAAATTGATGCCTGCGTGTGAGTGTTCCCTGGACCTGGACCAGC
AGCGGCAATATTACAGACCCGGGGGTTGGGGCAGACTGAGCCAAT
CTCTGCACCGTCAAAGTTATGGATACAGAGCCCTGGAAAAAGGCT
GAAGGATAAGATAGCTGACATTTATGAAGTGCTTCATTCATGTAG
CAGTGGGCCAAATGCGTACTTTACACTTGAGGAAGCTGAGGCTGG
AGGTTGATAACATGCCTCAAGTCTTCTAGAGTTAAATAACTTTGA
CCCAGGACCCAAGCCCAGAGTTCTGACTCAAAAACTAGGCCTCCT
AAACATCCTCTTATATGAGGTTAAATTTCATCTTCCTCTGTTTGG
CCTTGGCCTGGTTGGTGGATGCTCTGCTTCGGGGACCCAGGGCCA
GATGACAATGGGTTCTTTGTGCCCTTCAGACAATGGGAAGGGCTG
CCTGGGGAAAGATACAGTAACAAGGCAACAGGCTGAGTCAGCCTC
CAATGTGCTTGAACCTTCTTAGCTTGGCAGCCTTGACATTCAGCC
AGCCACACAAAGGGTATATCAAGGATGATACCACTAGTAGCAGCT
TGTCTTGTCTGTACCTCTGAACAAGAAAGAGGCTGTTCTGGGTCA
TCCCTCCAGGCCTGTCCAGCCCTGGCACTCTGTGAGTCGGTTTAG
GCAGCAGCCCCGGAACAGATGAGGCAGGCAGGGTTGGGACGTTTG
GTCAGGACAGCCCACCAGGAGGAAAGAAATGAAAGACAGAGACAG
CTTTGGCTATGGGAGAAGGAGGAGGCCGGGGGAAGGAGGAGACAG
GAGGAGGAGGGACCACGGGGTGGAGGGGAGATAGACCCAGCCCA

In some aspects, the inner ear supporting cell selective promoter is a TSPAN8 promoter. In some aspects, the TSPAN8 promoter comprises a nucleic acid sequence with at least 85%, at least 90%, at least 95%, at least 96%, at least 97%, at least 98% at least 99%, or 100% identity to SEQ ID NO: 98. In some aspects, the TSPAN8 promoter comprises the nucleic acid sequence of SEQ ID NO: 98.

Exemplary TSPAN8 promoter
(SEQ ID NO: 98)
CCAAGGACTCTTTTTTCTAAACTTCCCTTCATCTTCTAGTTTGAC
GCCCTTGGTGGGAAAAGTGTCTGAGATAAGGAAAAGGCATCCTTT
CAGTTCTCTGATACTATCTTGAAGCGAGGGATGGAGAAAGGCAAA
GAGAGACACAGGAGAAGCGTATCCCCTGGGAACAGGTGTCTAGTG
GAGTCCAGTAACTCACAGTCTCTCAGTTCCGTCAGCACTGTCCCT
TGGGTCGCAAATTTCTTCCATTAGCCCTTCCACCAGCTGTATTTC
AAATGGGGCTGGACAATAATTGTGGCCAGTGGCCTTGTGTTGTTT
GTACTTGCGGACTAGTAGTTCTCACCTGTCTTTCTCTGACTCCTA
TTAGCCACTGGGATTTCAGCAGCTGGTTCAGCCAATTCTACTCAA
TTCAACATTAAGTTGCAGTGGGCTAGAACTCATGGGCCGATTTAA
CAAGTGAAATTCTACCAAGATACATCAAAAATAGCAACAGGACTA
GATACTCAGCTCATTTTGTTTTATTTGTAATATACCAGTTGTGGC
TTTAGTGCCAGTCTGATTCATCTCTCTACTACAAAATGAGGCTCT
ATAAAGGAAAATATTGCAACTGGAGTGAGGAATTTGAATCTTATA
GGAAGGAATTTGTCTTCTCATGAAGACTTCAGTTTACCAGAAGTA
TCTATTGAGGAAGTGTTTACAAGAAAATGTGCCATTTAGCTTTAT
TCTAAATTTGCATAATAACTGAACCAAACAAAAAAATATAGATAG
ATAGATTGTTCTATCTATAGATAGATAGGGAACATTGGCAGTAGG
TGGCAGTAAGTTCCCCTGAGCACATGGAGGACACAGTTAAATGCA
TTTGAGGTATGTGGGAAATGGTTTAAAGCAGAATTTTATGCCAAC
TTTTAGTAACGGAAGCCTAACAAATGTTTGTTCTTTCAAGTGAGA
GAAGCAAGCAATCTGGAACTATTCATAAGCTTATTTTCTGTATCC
TTAAACATATTTTATAATGAATGTATGATTTAAATAGTAAGTTAA
GTGTCTGGGGGTACTGCACACCTCCCTTGCATACAGTCAAACTTC
TTCAGGGTGATGGGGAAGAGGAGTTATAGGCTGCCAAGCAAAATT
GCCAAACTGGTCTCAGAAATTCACTGCATTGGAGAGCGCGGGATC
CTTGCAACACTGACTTTAGCAGTTAAACTAGAGTGGTTGGGGATG
AGTATTCT

In some aspects, the inner ear supporting cell selective promoter is a VIM promoter. In some aspects, the VIM promoter comprises a nucleic acid sequence with at least 85%, at least 90%, at least 95%, at least 96%, at least 97%, at least 98% at least 99%, or 100% identity to SEQ ID NO: 99. In some aspects, the VIM promoter comprises the nucleic acid sequence of SEQ ID NO: 99.

Exemplary VIM promoter
(SEQ ID NO: 99)
ATTCACAATGCATTCCCTCTGCCCACCACATTAATTATCAACTCC
TTTTCCTGGCATTTACTCATCCAACGCATGGCCCCACGTTAACTT
TCAGTTCCCTTTCTCCCCTACAAATACTCCATAATCCAGCAACCC
TGGGATCCCTGAGATGATGAAGAGGACCAGTGCCCATTCCAGGAG
ACATCACCGCAGCCCTGAGGAATCGGCTATGGGCACCAGCAGGGC
ACAGTGCCACACCTCGCCAATGCCTTGTCCTCCTTTTCCATAGTG
AGTCAGTCAGCAAGCGTGTAGAAGTGAGTTCCACACTCTCTTCCT
CCCATAGGGAGATCACTTTTCTCATTCTAAGGGTTCCAGGCACAC
TCACAATGGTGGCATTTGCTGAGCAGTGGCTTGAATAAAGGGCTC
TCAGAAAGCAAGATGTAACTCAGAGCATAGGCTAGCCCCAGGAAT
GCTCTTGGGGAATGACCTGCAGCCTCCCAGTGAAAGAGAGAATAA
AAGAAAGCCCCAGCAGGCGAGCTGGGCAGTAGAGAGTCCTGTAAT
TCCACCTTGGCAAGCACCATTTGCAAGAACGAACTGGGATAAGGT
AAACAAAATATTGCCTAAAAGAGGCTTGTCCAAAGAAGTCAGAAT
ACGCTCTTCATTTACCTCTAAATTTCAGTACACCATAAATCTAAA
TACTCAAAAAAACCTGTGCCTTTTCAATCAAGGTCAATTGCACGA
ATTCTTTTGGAAAACAGGACCTATGGCATTTCCCAGACAAATCAC
CGTGAACCCTGTACTGTGCATTGCTGTCCTAAAATCCAAAGATTC
TGTCATTTGTGTTACATAATTGCCTTTCATTTGAACTCATTAATC
AAATTGGGGTTTTTAAGCAACACCTAATTAATTCTTTAACTGGCT
CATCCACTGATCACTGAGTTCTATTTTGAAACTACGGACGTCGAG
TTTCCTCTTTCACCCAGAATTTTCAGATCTTGTTTAAAAAGTTGG
GTGTGGTTTCATGGGGGGAGGGGGAAGAGCGAGAGGAGACCAGAG
GGACGGGGGCGGGGACTCTGCAAGAAAAACCTTCCCGGTGCAATC
GTGATCT

In some aspects, the inner ear supporting cell selective promoter is a GFAP promoter. In some aspects, the GFAP promoter comprises a nucleic acid sequence with at least 85%, at least 90%, at least 95%, at least 96%, at least 97%, at least 98% at least 99%, or 100% identity to SEQ ID NO: 91. In some aspects, the GFAP promoter comprises the nucleic acid sequence of SEQ ID NO: 91.

Exemplary GFAP promoter
(SEQ ID NO: 91)
GAACATATCCTGGTGTGGAGTAGGGGACGCTGCTCTGACAGAGGC
TCGGGGGCCTGAGCTGGCTCTGTGAGCTGGGGAGGAGGCAGACAG
CCAGGCCTTGTCTGCAAGCAGACCTGGCAGCATTGGGCTGGCCGC
CCCCCAGGGCCTCCTCTTCATGCCCAGTGAATGACTCACCTTGGC
ACAGACACAATGTTCGGGGTGGGCACAGTGCCTGCTTCCCGCCGC
ACCCCAGCCCCCCTCAAATGCCTTCCGAGAAGCCCATTGAGCAGG
GGGCTTGCATTGCACCCCAGCCTGACAGCCTGGCATCTTGGGATA
AAAGCAGCACAGCCCCCTAGGGGCTGCCCTTGCTGTGTGGCGCCA
CCGGCGGTGGAGAACAAGGCTCTATTCAGCCTGTGCCCAGGAAAG
GGGATCAGGGGATGCCCAGGCATGGACAGTGGGTGGCAGGGGGGG
AGAGGAGGGCTGTCTGCTTCCCAGAAGTCCAAGGACACAAATGGG
TGAGGGGAGCTCTCCCCATAGCTGGGCTGCGGCCCAACCCCACCC
CCTCAGGCTATGCCAGGGGGTGTTGCCAGGGGCACCCGGGCATCG
CCAGTCTAGCCCACTCCTTCATAAAGCCCTCGCATCCCAGGAGCG
AGCAGAGCCAGAGCAGGTTGGAGAGGAGACGCATCACCTCCGCTG
CTCGC

TABLE 2
Exemplary Promoters

| Promoter | SEQ ID NO |
|---|---|
| PARM1 derivative | 40 |
| GJB6 derivative | 16 |
| RBP7 derivative | 28 |
| IGFBP2 derivative | 57 |
| GDF6 derivative | 90 |
| GFAP derivative | 91 |
| BACE2 derivative | 92 |
| DBI2 derivative | 93 |
| FABP3 derivative | 94 |
| KLHL14 derivative | 95 |
| MMP15 derivative | 96 |
| SPARC derivative | 97 |
| TSPAN8 derivative | 98 |
| VIM derivative | 99 |
| GJB2 minimal promoter | 86 |
| 1. CAG enhancer/promoter | 15 |
| 2. CAG enhancer/promoter | 14 |
| 1. CMV/CBA enhancer/promoter | 13 |
| 2. CMV/CBA enhancer/promoter | 12 |
| 1. CBA promoter | 11 |
| 2. CBA promoter | 10 |

### Enhancers

In some instances, a construct can include an enhancer sequence. The term “enhancer” refers to a nucleotide sequence that can increase the level of transcription of a nucleic acid encoding a protein of interest (e.g., a polypeptide (e.g., a therapeutic polypeptide)). Enhancer sequences (generally 50-1500 bp in length) generally increase the level of transcription by providing additional binding sites for transcription-associated proteins (e.g., transcription factors). In some aspects, an enhancer sequence is found within an intronic sequence. Unlike promoter sequences, enhancer sequences can act at much larger distance away from the transcription start site (e.g., as compared to a promoter). Non-limiting examples of enhancers include a RSV enhancer, a CMV enhancer, and/or a SV40 enhancer. In some aspects, a construct comprises a CMV enhancer exemplified by SEQ ID NO: 18. In some aspects, a construct comprises a CMV enhancer exemplified by SEQ ID NO: 63. In some aspects, a construct comprises a chimeric intron enhancer exemplified by SEQ ID NO: 64. In some aspects, an enhancer sequence is at least 85%, at least 90%, at least 95%, at least 96%, at least 97%, at least 98% at least 99%, or 100% identical to the enhancer sequence represented by SEQ ID NO: 18. In some aspects, an enhancer sequence is at least 85%, at least 90%, at least 95%, at least 96%, at least 97%, at least 98% at least 99%, or 100% identical to the enhancer sequence represented by SEQ ID NO: 63. In some aspects, an enhancer sequence is at least 85%, at least 90%, at least 95%, at least 96%, at least 97%, at least 98% at least 99%, or 100% identical to the enhancer sequence represented by SEQ ID NO: 64. In some aspects, an enhancer sequence is at least 85%, at least 90%, at least 95%, at least 96%, at least 97%, at least 98% at least 99%, or 100% identical to the enhancer sequence represented by SEQ ID NO: 65. In some aspects, an SV-40 derived enhancer is the SV-40 T intron sequence, which is exemplified by SEQ ID NO: 19. In some aspects, an enhancer sequence is at least 85%, at least 90%, at least 95%, at least 96%, at least 97%, at least 98% at least 99%, or 100% identical to the enhancer sequence represented by SEQ ID NO: 19.

In some instances, the construct does not include an enhancer sequence.

Exemplary CMV enhancer
(SEQ ID NO: 18)
GACATTGATTATTGACTAGTTATTAATAGTAATCAATTACGGGGT
CATTAGTTCATAGCCCATATATGGAGTTCCGCGTTACATAACTTA
CGGTAAATGGCCCGCCTGGCTGACCGCCCAACGACCCCCGCCCAT
TGACGTCAATAATGACGTATGTTCCCATAGTAACGCCAATAGGGA
CTTTCCATTGACGTCAATGGGTGGACTATTTACGGTAAACTGCCC
ACTTGGCAGTACATCAAGTGTATCATATGCCAAGTACGCCCCCTA
TTGACGTCAATGACGGTAAATGGCCCGCCTGGCATTATGCCCAGT
ACATGACCTTATGGGACTTTCCTACTTGGCAGTACATCTACGTAT
TAGTCATCGCTATTACCATGG
Exemplary CMV enhancer
(SEQ ID NO: 63)
GACATTGATTATTGACTAGTTATTAATAGTAATCAATTACGGGGT
CATTAGTTCATAGCCCATATATGGAGTTCCGCGTTACATAACTTA
CGGTAAATGGCCCGCCTGGCTGACCGCCCAACGACCCCCGCCCAT
TGACGTCAATAATGACGTATGTTCCCATAGTAACGCCAATAGGGA
CTTTCCATTGACGTCAATGGGTGGACTATTTACGGTAAACTGCCC
ACTTGGCAGTACATCAAGTGTATCATATGCCAAGTACGCCCCCTA
TTGACGTCAATGACGGTAAATGGCCCGCCTGGCATTATGCCCAGT
ACATGACCTTATGGGACTTTCCTACTTGGCAGTACATCTACGTAT
TAGTCATCGCTATTACCATGGT
Exemplary SV-40 synthetic intron
(SEQ ID NO: 19)
GGAGTCGCTGCGTTGCCTTCGCCCCGTGCCCCGCTCCGCGCCGCC
TCGCGCCGCCCGCCCCGGCTCTGACTGACCGCGTTACTCCCACAG
GTGAGCGGGCGGGACGGCCCTTCTCCTCCGGGCTGTAATTAGCGC
TTGGTTTAATGACGGCTCGTTTCTTTTCTGTGGCTGCGTGAAAGC
CTTAAAGGGCTCCGGGAGGGCCCTTTGTGCGGGGGGGAGCGGCTC
GGGGGGTGCGTGCGTGTGTGTGTGCGTGGGGAGCGCCGCGTGCGG
CCCGCGCTGCCCGGCGGCTGTGAGCGCTGCGGGCGCGGCGCGGGG
CTTTGTGCGCTCCGCGTGTGCGCGAGGGGAGCGCGGCCGGGGGCG
GTGCCCCGCGGTGCGGGGGGGCTGCGAGGGGAACAAAGGCTGCGT
GCGGGGTGTGTGCGTGGGGGGGTGAGCAGGGGGTGTGGGCGCGGC
GGTCGGGCTGTAACCCCCCCCTGCACCCCCCTCCCCGAGTTGCTG
AGCACGGCCCGGCTTCGGGTGCGGGGCTCCGTGCGGGGCGTGGCG
CGGGGCTCGCCGTGCCGGGGGGGGGGTGGCGGCAGGTGGGGGTGC
CGGGCGGGGCGGGGCCGCCTCGGGCCGGGGAGGGCTCGGGGGAGG
GGCGCGGCGGCCCCCGGAGCGCCGGCGGCTGTCGAGGCGCGGCGA
GCCGCAGCCATTGCCTTTTATGGTAATCGTGCGAGAGGGCGCAGG
GACTTCCTTTGTCCCAAATCTGTGCGGAGCCGAAATCTGGGAGGC
GCCGCCGCACCCCCTCTAGCGGGCGCGGGGCGAAGCGGTGCGGCG
CCGGCAGGAAGGAAATGGGCGGGGAGGGCCTTCGTGCGTCGCCGC
GCCGCCGTCCCCTTCTCCCTCTCCAGCCTCGGGGCTGTCCGCGGG
GGGACGGCTGCCTTCGGGGGGGACGGGGCAGGGCGGGGTTCGGCT
TCTGGCGTGTGACCGGCGGCTCTAGAGCCTCTGCTAACCATGTTC
ATGCCTTCTTCTTTTTCCTACAG
Exemplary chimeric intron
(SEQ ID NO: 64)
GGAGTCGCTGCGTTGCCTTCGCCCCGTGCCCCGCTCCGCGCCGCC
TCGCGCCGCCCGCCCCGGCTCTGACTGACCGCGTTACTCCCACAG
GTGAGCGGGCGGGACGGCCCTTCTCCTCCGGGCTGTAATTAGCGC
TTGGTTTAATGACGGCTTGTTTCTTTTCTGTGGCTGCGTGAAAGC
CTTGAGGGGCTCCGGGAGCTAGAGCCTCTGCTAACCATGTTCATG
CCTTCTTCTTTTTCCTACAG

### Flanking Untranslated Regions, 5′ UTRs and 3′ UTRs

In some aspects, any of the constructs described herein can include an untranslated region (UTR), such as a 5′ UTR or a 3′ UTR. UTRs of a gene are transcribed but not translated. A 5′ UTR starts at the transcription start site and continues to the start codon but does not include the start codon. A 3′ UTR starts immediately following the stop codon and continues until the transcriptional termination signal. The regulatory and/or control features of a UTR can be incorporated into any of the constructs, compositions, kits, or methods as described herein to enhance or otherwise modulate the expression of a polypeptide (e.g., a therapeutic polypeptide).

Natural 5′ UTRs include a sequence that plays a role in translation initiation. in some aspects, a 5′ UTR can comprise sequences, like Kozak sequences, which are commonly known to be involved in the process by which the ribosome initiates translation of many genes. Kozak sequences have the consensus sequence CCR(A/G)CCAUGG, where R is a purine (A or G) three bases upstream of the start codon (AUG), and the start codon is followed by another “G”. The 5′ UTRs have also been known to form secondary structures that are involved in elongation factor binding.

In some aspects, a 5′ UTR is included in any of the constructs described herein. Non-limiting examples of 5′ UTRs, including those from the following genes: albumin, serum amyloid A, Apolipoprotein AB/E, transferrin, alpha fetoprotein, erythropoietin, and Factor VIII, can be used to enhance expression of a nucleic acid molecule, such as an mRNA.

In some aspects, a 5′ UTR from an mRNA that is transcribed by a cell in the cochlea can be included in any of the constructs, compositions, kits, and methods described herein. In some aspects, a 5′ UTR is derived from the endogenous GJB2 gene loci and may include all or part of the endogenous sequence exemplified by SEQ ID NO: 20, SEQ ID NO: 21, or SEQ ID NO: 66. In some aspects, a 5′ UTR sequence is at least 85%, at least 90%, at least 95%, at least 96%, at least 97%, at least 98% at least 99%, or 100% identical to the 5′ UTR sequence represented by SEQ ID NO: 20, SEQ ID NO: 21, or SEQ ID NO: 66.

3′ UTRs are found immediately 3′ to the stop codon of the gene of interest. In some aspects, a 3′ UTR from an mRNA that is transcribed by a cell in the cochlea can be included in any of the constructs, compositions, kits, and methods described herein. In some aspects, a 3′ UTR is derived from the endogenous GJB2 gene loci and may include all or part of the endogenous sequence exemplified by SEQ ID NO: 22. In some aspects, a 3′ UTR sequence is at least 85%, at least 90%, at least 95%, at least 96%, at least 97%, at least 98% at least 99%, or 100% identical to the 3′ UTR sequence represented by SEQ ID NO: 22. In some aspects, a 3′ UTR is derived from the endogenous GJB2 gene loci and may include all or part of the endogenous sequence exemplified by SEQ ID NO: 67, or SEQ ID NO: 68. In some aspects, a 3′ UTR sequence is at least 85%, at least 90%, at least 95%, at least 96%, at least 97%, at least 98% at least 99%, or 100% identical to the 3′ UTR sequence represented by SEQ ID NO: 67, or SEQ ID NO: 68.

3′ UTRs are known to have stretches of adenosines and uridines (in the RNA form) or thymidines (in the DNA form) embedded in them. These AU-rich signatures are particularly prevalent in genes with high rates of turnover. Based on their sequence features and functional properties, the AU-rich elements (AREs) can be separated into three classes (Chen et al., Mol. Cell. Biol. 15:5777-5788, 1995; Chen et al., Mol. Cell Biol. 15:2010-2018, 1995, each of which is incorporated herein by reference in its entirety): Class I AREs contain several dispersed copies of an AUUUA motif within U-rich regions. For example, c-Myc and MyoD mRNAs contain class I AREs. Class II AREs possess two or more overlapping UUAUUUA(U/A) (U/A) nonamers. GM-CSF and TNF-alpha mRNAs are examples that contain class II AREs. Class III AREs are less well defined. These U-rich regions do not contain an AUUUA motif, two well-studied examples of this class are c-Jun and myogenin mRNAs.

Most proteins binding to the AREs are known to destabilize the messenger, whereas members of the ELAV family, most notably HuR, have been documented to increase the stability of mRNA. HuR binds to AREs of all the three classes. Engineering the HuR specific binding sites into the 3′ UTR of nucleic acid molecules will lead to HuR binding and thus, stabilization of the message in vivo.

In some aspects, the introduction, removal, or modification of 3′ UTR AREs can be used to modulate the stability of an mRNA encoding a polypeptide (e.g., a therapeutic polypeptide). In other aspects, AREs can be removed or mutated to increase the intracellular stability and thus increase translation and production of a polypeptide (e.g., a therapeutic polypeptide).

In other aspects, non-ARE sequences may be incorporated into the 5′ or 3′ UTRs. In some aspects, introns or portions of intron sequences may be incorporated into the flanking regions of the polynucleotides in any of the constructs, compositions, kits, and methods provided herein. Incorporation of intronic sequences may increase protein production as well as mRNA levels.

Exemplary 5′ UTR Sequence
(SEQ ID NO: 20)
GTTGCGGCCCCGCAGCGCCCGCGCGCTCCTCTCCCCGACTCGGAG
CCCCTCGGCGGCGCCCGGCCCAGGACCCGCCTAGGAGCGCAGGAG
CCCCAGCGCAGAGACCCCAACGCCGAGACCCCCGCCCCGGCCCCG
CCGCGCTTCCTCCCGACGCAGAGCAAACCGCCCAGAGTAGAAG
Exemplary 5′ UTR Sequence
(SEQ ID NO: 21)
TTTAGGACCCTTGTTCGCGAAGAGGTGGTGTGCGGCTGAGACCCG
CGTCCTCAGGACGGTTCCATCAGTGCCTCGATCCTGCCCCACTGG
AGGAGGAAGGCAGCCCGAACAGCGCTCACCTAACTAACAGCTGCT
GAGAGCTGGGTTCCGTGGCCATGCACCTGGGACTGCCTTGAGAAG
CGTGAGCAAACCGCCCAGAGTAGAAG
Exemplary 5′ UTR Sequence
(SEQ ID NO: 66)
GTTGCGGCCCCGCAGCGCCCGCGCGCTCCTCTCCCCGACTCGGAG
CCCCTCGGCGGCGCCCGGCCCAGGACCCGCCTAGGAGCGCAGGAG
CCCCAGCGCAGAGACCCCAACGCCGAGACCCCCGCCCCGGCCCCG
CCGCGCTTCCTCCCGACGCAGTTTAGGACCCTTGTTCGCGAAGAG
GTGGTGTGCGGCTGAGACCCGCGTCCTCAGGACGGTTCCATCAGT
GCCTCGATCCTGCCCCACTGGAGGAGGAAGGCAGCCCGAACAGCG
CTCACCTAACTAACAGCTGCTGAGAGCTGGGTTCCGTGGCCATGC
ACCTGGGACTGCCTTGAGAAGCGTGAGCAAACCGCCCAGAGTAGA
AG
Exemplary 3′ UTR Sequence
(SEQ ID NO: 22)
CGCATTGCCCAGTTGTTAGATTAAGAAATAGACAGCATGAGAGGG
ATGAGGCAACCCGTGCTCAGCTGTCAAGGCTCAGTCGCTAGCATT
TCCCAACACAAAGATTCTGACCTTAAATGCAACCATTTGAAACCC
CTGTAGGCCTCAGGTGAAACTCCAGATGCCACAATGGAGCCTCTG
CTCCCCTAAAGCCTCAAAACAAAGGCCTAATTCTATGCCTGTCTT
AATTTTCTTTCACTTAAGTTAGTTCCACTGAGACCCCAGGCTGTT
AGGGGTTATTGGTGTAAGGTACTTTCATATTTTAAACAGAGGATA
TCGGCATTTGTTTCTTTCTCTGAGGACAAGAGAAAAAAGCCAGGT
TCCACAGAGGACACAGAGAAGGTTTGGGTGTCCTCCTGGGGTTCT
TTTTGCCAACTTTCCCCACGTTAAAGGTGAACATTGGTTCTTTCA
TTTGCTTTGGAAGTTTTAATCTCTAACAGTGGACAAAGTTACCAG
TGCCTTAAACTCTGTTACACTTTTTGGAAGTGAAAACTTTGTAGT
ATGATAGGTTATTTTGATGTAAAGATGTTCTGGATACCATTATAT
GTTCCCCCTGTTTCAGAGGCTCAGATTGTAATATGTAAATGGTAT
GTCATTCGCTACTATGATTTAATTTGAAATATGGTCTTTTGGTTA
TGAATACTTTGCAGCACAGCTGAGAGGCTGTCTGTTGTATTCATT
GTGGTCATAGCACCTAACAACATTGTAGCCTCAATCGAGTGAGAC
AGACTAGAAGTTCCTAGTGATGGCTTATGATAGCAAATGGCCTCA
TGTCAAATATTTAGATGTAATTTTGTGTAAGAAATACAGACTGGA
TGTACCACCAACTACTACCTGTAATGACAGGCCTGTCCAACACAT
CTCCCTTTTCCATGACTGTGGTAGCCAGCATCGGAAAGAACGCTG
ATTTAAAGAGGTCGCTTGGGAATTTTATTGACACAGTACCATTTA
ATGGGGAGGACAAAATGGGGCAGGGGAGGGAGAAGTTTCTGTCGT
TAAAAACAGATTTGGAAAGACTGGACTCTAAAGTCTGTTGATTAA
AGATGAGCTTTGTCTACTTCAAAAGTTTGTTTGCTTACCCCTTCA
GCCTCCAATTTTTTAAGTGAAAATATAGCTAATAACATGTGAAAA
GAATAGAAGCTAAGGTTTAGATAAATATTGAGCAGATCTATAGGA
AGATTGAACCTGAATATTGCCATTATGCTTGACATGGTTTCCAAA
AAATGGTACTCCACATATTTCAGTGAGGGTAAGTATTTTCCTGTT
GTCAAGAATAGCATTGTAAAAGCATTTTGTAATAATAAAGAATAG
CTTTAATGATATGCTTGTAACTAAAATAATTTTGTAATGTATCAA
ATACATTTAAAACATTAAAATATAATCTCTATAATAA
Exemplary 3′ UTR Sequence
(SEQ ID NO: 67)
GAAATAGACAGCATGAGAGGGATGAGGCAACCCGTGCTCAGCTGT
CAAGGCTCAGTCGCTAGCATTTCCCAACACAAAGATTCTGACCTT
AAATGCAACCATTTGAAACCCCTGTAGGCCTCAGGTGAAACTCCA
GATGCCACAATGGAGCTCTGCTCCCCTAAAGCCTCAAAACAAAGG
CCTAATTCTATGCCTGTCTTAATTTTCTTTCACTTAAGTTAGTTC
CACTGAGACCCCAGGCTGTTAGGGGTTATTGGTGTAAGGTACTTT
CATATTTTAAACAGAGGATATCGGCATTTGTTTCTTTCTCTGAGG
ACAAGAGAAAAAAGCCAGGTTCCACAGAGGACACAGAGAAGGTTT
GGGTGTCCTCCTGGGGTTCTTTTTGCCAACTTTCCCCACGTTAAA
GGTGAACATTGGTTCTTTCATTTGCTTTGGAAGTTTTAATCTCTA
ACAGTGGACAAAGTTACCAGTGCCTTAAACTCTGTTACACTTTTT
GGAAGTGAAAACTTTGTAGTATGATAGGTTATTTTGATGTAAAGA
TGTTCTGGATACCATTATATGTTCCCCCTGTTTCAGAGGCTCAGA
TTGTAATATGTAAATGGTATGTCATTCGCTACTATGATTTAATTT
GAAATATGGTCTTTTGGTTATGAATACTTTGCAGCACAGCTGAGA
GGCTGTCTGTTGTATTCATTGTGGTCATAGCACCTAACAACATTG
TAGCCTCAATCGAGTGAGACAGACTAGAAGTTCCTAGTGATGGCT
TATGATAGCAAATGGCCTCATGTCAAATATTTAGATGTAATTTTG
TGTAAGAAATACAGACTGGATGTACCACCAACTACTACCTGTAAT
GACAGGCCTGTCCAACACATCTCCCTTTTCCATGACTGTGGTAGC
CAGCATCGGAAAGAACGCTGATTTAAAGAGGTCGCTTGGGAATTT
TATTGACACAGTACCATTTAATGGGGAGGACAAAATGGGGCAGGG
GAGGGAGAAGTTTCTGTCGTTAAAAACAGATTTGGAAAGACTGGA
CTCTAAAGTCTGTTGATTAAAGATGAGCTTTGTCTACTTCAAAAG
TTTGTTTGCTTACCCCTTCAGCCTCCAATTTTTTAAGTGAAAATA
TAGCTAATAACATGTGAAAAGAATAGAAGCTAAGGTTTAGATAAA
TATTGAGCAGATCTATAGGAAGATTGAACCTGAATATTGCCATTA
TGCTTGACATGGTTTCCAAAAAATGGTACTCCACATATTTCAGTG
AGGGTAAGTATTTTCCTGTTGTCAAGAATAGCATTGTAAAAGCAT
TTTGTAATAATAAAGAATAGCTTTAATGATATGCTTGTAACTAAA
ATAATTTTGTAATGTATCAAATACATTTAAAACATTAAAATATAA
TCTCTATAATAA
Exemplary 3′ UTR Sequence
(SEQ ID NO: 68)
CGCATTGCCCAGTTGTTAGATTAAGAAATAGACAGCATGAGAGGG
ATGAGGCAACCCGTGCTCAGCTGTCAAGGCTCAGTCGCTAGCATT
TCCCAACACAAAGATTCTGACCTTAAATGCAACCATTTGAAACCC
CTGTAGGCCTCAGGTGAAACTCCAGATGCCACAATGGAGCCTCTG
CTCCCCTAAAGCCTCAAAACAAAGGCCTAATTCTATGCCTGTCTT
AATTTTCTTTCACTTAAGTTAGTTCCACTGAGACCCCAGGCTGTT
AGGGGTTATTGGTGTAAGGTACTTTCATATTTTAAACAGAGGATA
TCGGCATTTGTTTCTTTCTCTGAGGACAAGAGAAAAAAGCCAGGT
TCCACAGAGGACACAGAGAAGGTTTGGGTGTCCTCCTGGGGTTCT
TTTTGCCAACTTTCCCCACGTTAAAGGTGAACATTGGTTCTTTCA
TTTGCTTTGGAAGTTTTAATCTCTAACAGTGGACAAAGTTACCAG
TGCCTTAAACTCTGTTACACTTTTTGGAAGTGAAAACTTTGTAGT
ATGATAGGTTATTTTGATGTAAAGATGTTCTGGATACCATTATAT
GTTCCCCCTGTTTCAGAGGCTCAGATTGTAATATGTAAATGGTAT
GTCATTCGCTACTATGATTTAATTTGAAATATGGTCTTTTGGTTA
TGAATACTTTGCAGCACAGCTGAGAGGCTGTCTGTTGTATTCATT
GTGGTCATAGCACCTAACAACATTGTAGCCTCAATCGAGTGAGAC
AGACTAGAAGTTCCTAGTGATGGCTTATGATAGCAAATGGCCTCA
TGTCAAATATTTAGATGTAATTTTGTGTAAGAAATACAGACTGGA
TGTACCACCAACTACTACCTGTAATGACAGGCCTGTCCAACACAT
CTCCCTTTTCCATGACTGTGGTAGCCAGCATCGGAAAGAACGCTG
ATTTAAAGAGGTCGCTTGGGAATTTTATTGACACAGTACCATTTA
ATGGGGAGGACAAAATGGGGCAGGGGAGGGAGAAGTTTCTGTCGT
TAAAAACAGATTTGGAAAGACTGGACTCTAAAGTCTGTTGATTAA
AGATGAGCTTTGTCTACTTCAAAAGTTTGTTTGCTTACCCCTTCA
GCCTCCAATTTTTTAAGTGAAAATATAGCTAATAACATGTGAAAA
GAATAGAAGCTAAGGTTTAGATAAATATTGAGCAGATCTATAGGA
AGATTGAACCTGAATATTGCCATTATGCTTGAC
Exemplary 3′ UTR Sequence
(SEQ ID NO: 69)
GAGCTCAGTGTGAGTTCTACCATTGCCAAACTCGAGCAGTGAATT
CTACCAGTGCCATAGGATCCAGTGTGAGTTCTACCATTGCCAAAG
GTACCCAGTGAATTCTACCAGTGCCATAGTTAACCGCATTGCCCA
GTTGTTAGATTAAGAAATAGACAGCATGAGAGGGATGAGGCAACC
CGTGCTCAGCTGTCAAGGCTCAGTCGCTAGCATTTCCCAACACAA
AGATTCTGACCTTAAATGCAACCATTTGAAACCCCTGTAGGCCTC
AGGTGAAACTCCAGATGCCACAATGGAGCCTCTGCTCCCCTAAAG
CCTCAAAACAAAGGCCTAATTCTATGCCTGTCTTAATTTTCTTTC
ACTTAAGTTAGTTCCACTGAGACCCCAGGCTGTTAGGGGTTATTG
GTGTAAGGTACTTTCATATTTTAAACAGAGGATATCGGCATTTGT
TTCTTTCTCTGAGGACAAGAGAAAAAAGCCAGGTTCCACAGAGGA
CACAGAGAAGGTTTGGGTGTCCTCCTGGGGTTCTTTTTGCCAACT
TTCCCCACGTTAAAGGTGAACATTGGTTCTTTCATTTGCTTTGGA
AGTTTTAATCTCTAACAGTGGACAAAGTTACCAGTGCCTTAAACT
CTGTTACACTTTTTGGAAGTGAAAACTTTGTAGTATGATAGGTTA
TTTTGATGTAAAGATGTTCTGGATACCATTATATGTTCCCCCTGT
TTCAGAGGCTCAGATTGTAATATGTAAATGGTATGTCATTCGCTA
CTATGATTTAATTTGAAATATGGTCTTTTGGTTATGAATACTTTG
CAGCACAGCTGAGAGGCTGTCTGTTGTATTCATTGTGGTCATAGC
ACCTAACAACATTGTAGCCTCAATCGAGTGAGACAGACTAGAAGT
TCCTAGTGATGGCTTATGATAGCAAATGGCCTCATGTCAAATATT
TAGATGTAATTTTGTGTAAGAAATACAGACTGGATGTACCACCAA
CTACTACCTGTAATGACAGGCCTGTCCAACACATCTCCCTTTTCC
ATGACTGTGGTAGCCAGCATCGGAAAGAACGCTGATTTAAAGAGG
TCGCTTGGGAATTTTATTGACACAGTACCATTTAATGGGGAGGAC
AAAATGGGGCAGGGGAGGGAGAAGTTTCTGTCGTTAAAAACAGAT
TTGGAAAGACTGGACTCTAAAGTCTGTTGATTAAAGATGAGCTTT
GTCTACTTCAAAAGTTTGTTTGCTTACCCCTTCAGCCTCCAATTT
TTTAAGTGAAAATATAGCTAATAACATGTGAAAAGAATAGAAGCT
AAGGTTTAGATAAATATTGAGCAGATCTATAGGAAGATTGAACCT
GAATATTGCCATTATGCTTGAC

microRNA Regulatory Target Sites (miRTS)

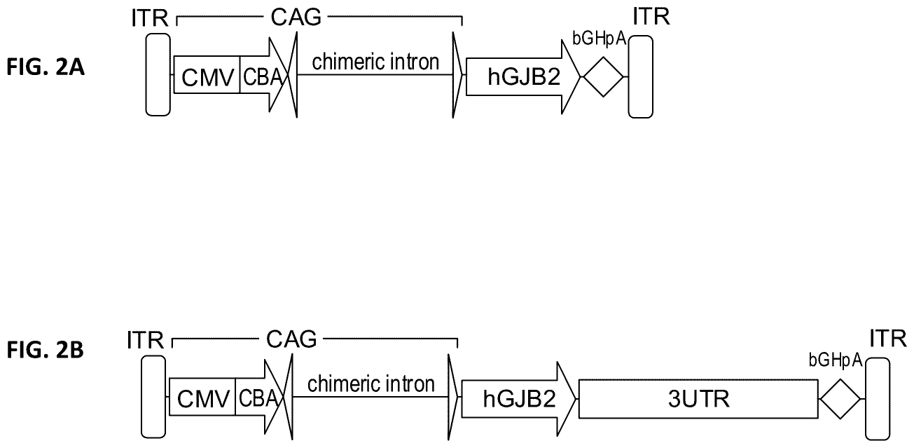

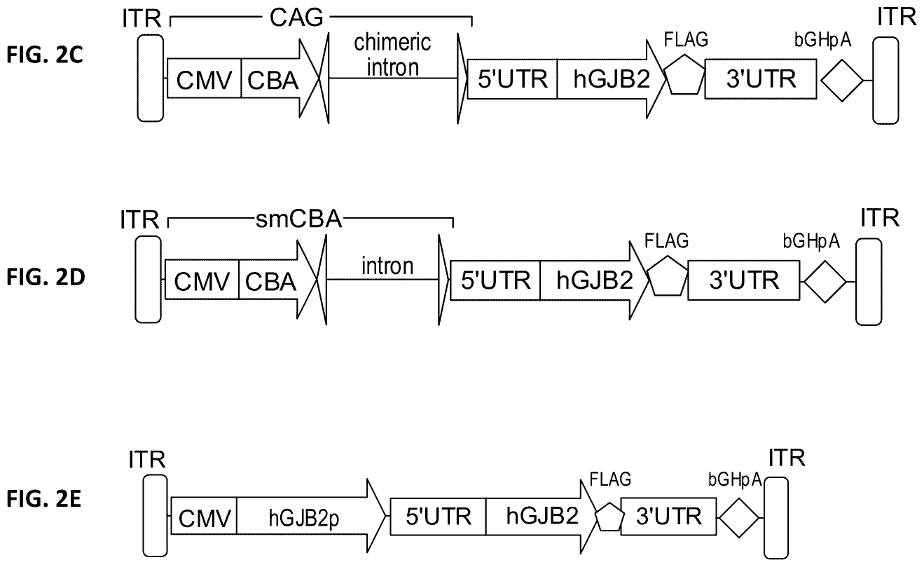

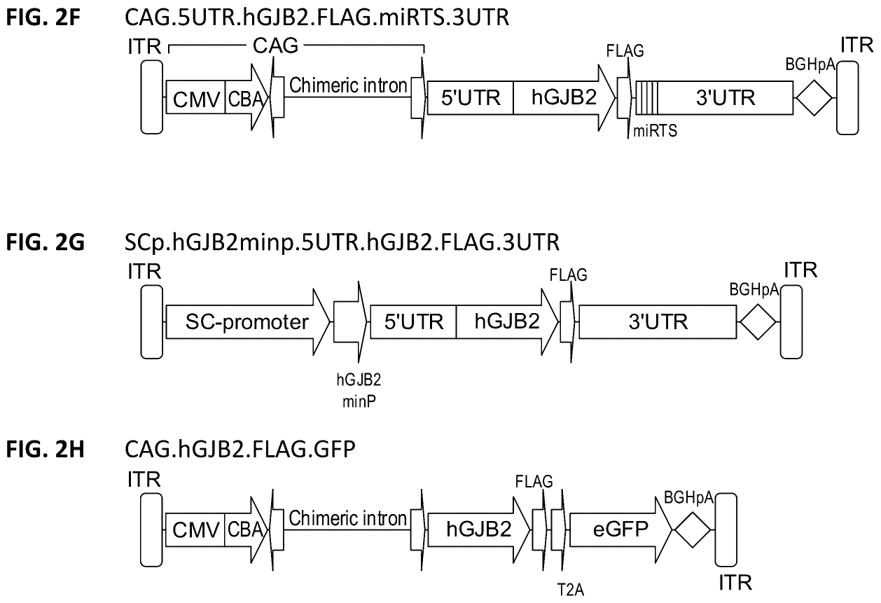

In some aspects, the disclosure is directed to constructs comprising microRNA regulatory target site (miRTS) which can be used to regulate (e.g., reduce) expression of a polynucleotide encoding a therapeutic polypeptide in a cell (e.g., an inner ear cell, e.g., a hair cell). In some aspects, the constructs provide reduced toxicity associated with expression of the therapeutic polypeptide in some cells (e.g., an inner ear cell, e.g., a hair cell). In some aspects, the construct comprising a polynucleotide encoding a therapeutic polypeptide comprises a microRNA regulatory target site (miRTS). An exemplary polynucleotide construct comprising a miRTS is provided in FIG. 2F. In some aspects, a UTR may comprise miRTS. In some aspects, a 3′ UTR may comprise a miRTS. In some aspects, a 5′ UTR may comprise a miRTS.

In some aspects, expression of the therapeutic polypeptide is reduced, suppressed, inhibited, or eliminated in cells that express the microRNA. In some aspects, the therapeutic polypeptide is predominately expressed in cells that do not express the microRNA. In some aspects, toxicity associated with the expression of the therapeutic polypeptide is reduced, suppressed, inhibited, or eliminated in cells that express the microRNA.

In some aspects, the disclosure is directed to constructs comprising microRNA regulatory target site (miRTS) which can be used to regulate (e.g., reduce) expression of a polynucleotide encoding a polypeptide in a cell (e.g., an inner ear cell, e.g., a hair cell). In some aspects, the constructs provide reduced toxicity associated with expression of the polypeptide in some cells (e.g., an inner ear cell, e.g., a hair cell). In some aspects, the construct comprising a polynucleotide encoding a polypeptide comprises a microRNA regulatory target site (miRTS). An exemplary polynucleotide construct comprising a miRTS is provided in FIG. 2F. In some aspects, a UTR may comprise miRTS. In some aspects, a 3′ UTR may comprise a miRTS. In some aspects, a 5′ UTR may comprise a miRTS.

In some aspects, expression of the polypeptide is reduced, suppressed, inhibited, or eliminated in cells that express the microRNA. In some aspects, the polypeptide is predominately expressed in cells that do not express the microRNA. In some aspects, toxicity associated with the expression of the polypeptide is reduced, suppressed, inhibited, or eliminated in cells that express the microRNA.

In some aspects, the miRTS is specific microRNAs expressed in inner ear hair cells, spiral ganglion cells, lateral supporting cells, basilar membrane cells, medial supporting cells, spiral limbus cells, or any combination thereof. In some aspects, the miRTS is specific microRNAs expressed in inner ear hair cells. In some aspects, the miRTS is specific microRNAs expressed in spiral ganglion cells. In some aspects, the miRTS is specific microRNAs expressed in lateral supporting cells. In some aspects, the miRTS is specific microRNAs expressed in basilar membrane cells. In some aspects, the miRTS is specific microRNAs expressed in medial supporting cells. In some aspects, the miRTS is specific microRNAs expressed in spiral limbus cells.

In some aspects, the miRTS may be a human miRNA-182, miRNA-183, miRNA-194, miRNA-140, miRNA-18a, miRNA-99a, miRNA-30b, miRNA-15a target sequence. In some aspects, a miRTS may be a human miRNA-182 target sequence. In some aspects, a UTR may include all or part of the miRNA-182 target sequence. In some aspects, a UTR may contain more than one miRNA-182 target sequence. In some aspects, more than one miRNA-182 target sequences may be dispersed at multiple locations in a UTR. In some aspects, the 3′ UTR may include all or part of the miRNA-182 target sequence. In some aspects, the 3′ UTR may contain more than one miRNA-182 target sequence. In some aspects, more than one miRNA-182 target sequences may be dispersed at multiple locations in the 3′ UTR. In some aspects, the miRNA-182 target sequence comprises the nucleic acid sequence with at least 85%, at least 90%, at least 95%, at least 96%, at least 97%, at least 98%, at least 99%, or 100% identity to SEQ ID NO: 78. In some aspects, the miRNA-182 target sequence comprises the nucleic acid sequence of SEQ ID NO: 78.

In some aspects, a miRTS may be a human miRNA-183 target sequence. In some aspects, a UTR may include all or part of the miRNA-183 target sequence. In some aspects, a UTR may contain more than one miRNA-183 target sequence. In some aspects, more than one miRNA-183 target sequences may be dispersed at multiple locations in a UTR. In some aspects, the 3′ UTR may include all or part of the miRNA-183 target sequence. In some aspects, the 3′ UTR may contain more than one miRNA-183 target sequence. In some aspects, more than one miRNA-183 target sequences may be dispersed at multiple locations in the 3′ UTR. In some aspects, the miRNA-183 target sequence comprises the nucleic acid sequence with at least 85%, at least 90%, at least 95%, at least 96%, at least 97%, at least 98%, at least 99%, or 100% identity to SEQ ID NO: 79. In some aspects, the miRNA-183 target sequence comprises the nucleic acid sequence of SEQ ID NO: 79.

In some aspects, a miRTS may be a human miRNA-194 target sequence. In some aspects, a UTR may include all or part of the miRNA-194 target sequence. In some aspects, a UTR may contain more than one miRNA-194 target sequence. In some aspects, more than one miRNA-194 target sequences may be dispersed at multiple locations in a UTR. In some aspects, the 3′ UTR may include all or part of the miRNA-194 target sequence. In some aspects, the 3′ UTR may contain more than one miRNA-194 target sequence. In some aspects, more than one miRNA-194 target sequences may be dispersed at multiple locations in the 3′ UTR. In some aspects, the miRNA-194 target sequence comprises the nucleic acid sequence with at least 85%, at least 90%, at least 95%, at least 96%, at least 97%, at least 98%, at least 99%, or 100% identity to SEQ ID NO: 1. In some aspects, the miRNA-194 target sequence comprises the nucleic acid sequence of SEQ ID NO: 1.

In some aspects, a miRTS may be a human miRNA-140 target sequence. In some aspects, a UTR may include all or part of the miRNA-140 target sequence. In some aspects, a UTR may contain more than one miRNA-140 target sequence. In some aspects, more than one miRNA-140 target sequences may be dispersed at multiple locations in a UTR. In some aspects, the 3′ UTR may include all or part of the miRNA-140 target sequence. In some aspects, the 3′ UTR may contain more than one miRNA-140 target sequence. In some aspects, more than one miRNA-140 target sequences may be dispersed at multiple locations in the 3′ UTR. In some aspects, the miRNA-140 target sequence comprises the nucleic acid sequence with at least 85%, at least 90%, at least 95%, at least 96%, at least 97%, at least 98%, at least 99%, or 100% identity to SEQ ID NO: 2. In some aspects, the miRNA-140 target sequence comprises the nucleic acid sequence of SEQ ID NO: 2.

In some aspects, a miRTS may be a human miRNA-18a target sequence. In some aspects, a UTR may include all or part of the miRNA-18a target sequence. In some aspects, a UTR may contain more than one miRNA-18a target sequence. In some aspects, more than one miRNA-18a target sequences may be dispersed at multiple locations in a UTR. In some aspects, the 3′ UTR may include all or part of the miRNA-18a target sequence. In some aspects, the 3′ UTR may contain more than one miRNA-18a target sequence. In some aspects, more than one miRNA-18a target sequences may be dispersed at multiple locations in the 3′ UTR. In some aspects, the miRNA-18a target sequence comprises the nucleic acid sequence with at least 85%, at least 90%, at least 95%, at least 96%, at least 97%, at least 98%, at least 99%, or 100% identity to SEQ ID NO: 3. In some aspects, the miRNA-18a target sequence comprises the nucleic acid sequence of SEQ ID NO: 3.

In some aspects, a miRTS may be a human miRNA-99a target sequence. In some aspects, a UTR may include all or part of the miRNA-99a target sequence. In some aspects, a UTR may contain more than one miRNA-99a target sequence. In some aspects, more than one miRNA-99a target sequences may be dispersed at multiple locations in a UTR. In some aspects, the 3′ UTR may include all or part of the miRNA-99a target sequence. In some aspects, the 3′ UTR may contain more than one miRNA-99a target sequence. In some aspects, more than one miRNA-99a target sequences may be dispersed at multiple locations in the 3′ UTR. In some aspects, the miRNA-99a target sequence comprises the nucleic acid sequence with at least 85%, at least 90%, at least 95%, at least 96%, at least 97%, at least 98%, at least 99%, or 100% identity to SEQ ID NO: 4. In some aspects, the miRNA-99a target sequence comprises the nucleic acid sequence of SEQ ID NO: 4.

In some aspects, a miRTS may be a human miRNA-30b target sequence. In some aspects, a UTR may include all or part of the miRNA-30b target sequence. In some aspects, a UTR may contain more than one miRNA-30b target sequence. In some aspects, more than one miRNA-30b target sequences may be dispersed at multiple locations in a UTR. In some aspects, the 3′ UTR may include all or part of the miRNA-30b target sequence. In some aspects, the 3′ UTR may contain more than one miRNA-30b target sequence. In some aspects, more than one miRNA-30b target sequences may be dispersed at multiple locations in the 3′ UTR. In some aspects, the miRNA-30b target sequence comprises the nucleic acid sequence with at least 85%, at least 90%, at least 95%, at least 96%, at least 97%, at least 98%, at least 99%, or 100% identity to SEQ ID NO: 5. In some aspects, the miRNA-30b target sequence comprises the nucleic acid sequence of SEQ ID NO: 5.

In some aspects, a miRTS may be a human miRNA-15a target sequence. In some aspects, a UTR may include all or part of the miRNA-15a target sequence. In some aspects, a UTR may contain more than one miRNA-15a target sequence. In some aspects, more than one miRNA-15a target sequences may be dispersed at multiple locations in a UTR. In some aspects, the 3′ UTR may include all or part of the miRNA-15a target sequence. In some aspects, the 3′ UTR may contain more than one miRNA-15a target sequence. In some aspects, more than one miRNA-15a target sequences may be dispersed at multiple locations in the 3′ UTR. In some aspects, the miRNA-15a target sequence comprises the nucleic acid sequence with at least 85%, at least 90%, at least 95%, at least 96%, at least 97%, at least 98%, at least 99%, or 100% identity to SEQ ID NO: 6. In some aspects, the miRNA-15a target sequence comprises the nucleic acid sequence of SEQ ID NO: 6.

In some aspects, the miRTS may be a target sequence for a miRNA that is expressed in specific cells of the inner ear. In some aspects, the miRTS may be a target sequence for a miRNA that is expressed in inner ear hair cells, spiral ganglion cells, lateral supporting cells, basilar membrane cells, medial supporting cells, spiral limbus cells, or any combination thereof.

In some aspects, the miRTS may be a target sequence for a miRNA that is expressed in inner ear hair cells. In some aspects, the miRNA that is expressed in inner ear hair cells reduces, suppresses, inhibits, or eliminates expression of the polypeptide (e.g., a therapeutic polypeptide) in the inner ear hair cells. In some aspects, miRNAs that are expressed in inner ear hair cells are miR-194, miR-140, miR-18a, miR-99a, miR-30b, miR-15a, miR182, or miR-183. In some aspects, the miRNA that is expressed in inner ear hair cells is miR-194. In some aspects, the miRNA-194 target sequence comprises the nucleic acid sequence with at least 85%, at least 90%, at least 95%, at least 96%, at least 97%, at least 98%, at least 99%, or 100% identity to SEQ ID NO: 1. In some aspects, the miRNA-194 target sequence comprises the nucleic acid sequence of SEQ ID NO: 1. In some aspects, the miRNA that is expressed in inner ear hair cells is miR-140. In some aspects, the miRNA-140 target sequence comprises the nucleic acid sequence with at least 85%, at least 90%, at least 95%, at least 96%, at least 97%, at least 98%, at least 99%, or 100% identity to SEQ ID NO: 2. In some aspects, the miRNA-140 target sequence comprises the nucleic acid sequence of SEQ ID NO: 2. In some aspects, the miRNA that is expressed in inner ear hair cells is miR-18a. In some aspects, the miRNA-18a target sequence comprises the nucleic acid sequence with at least 85%, at least 90%, at least 95%, at least 96%, at least 97%, at least 98%, at least 99%, or 100% identity to SEQ ID NO: 3. In some aspects, the miRNA-18a target sequence comprises the nucleic acid sequence of SEQ ID NO: 3. In some aspects, the miRNA that is expressed in inner ear hair cells is miR-99a. In some aspects, the miRNA-99a target sequence comprises the nucleic acid sequence with at least 85%, at least 90%, at least 95%, at least 96%, at least 97%, at least 98%, at least 99%, or 100% identity to SEQ ID NO: 4. In some aspects, the miRNA-99a target sequence comprises the nucleic acid sequence of SEQ ID NO: 4. In some aspects, the miRNA that is expressed in inner ear hair cells is miR-30b. In some aspects, the miRNA-30b target sequence comprises the nucleic acid sequence with at least 85%, at least 90%, at least 95%, at least 96%, at least 97%, at least 98%, at least 99%, or 100% identity to SEQ ID NO: 5. In some aspects, the miRNA-30b target sequence comprises the nucleic acid sequence of SEQ ID NO: 5. In some aspects, the miRNA that is expressed in inner ear hair cells is miR-15a. In some aspects, the miRNA-15a target sequence comprises the nucleic acid sequence with at least 85%, at least 90%, at least 95%, at least 96%, at least 97%, at least 98%, at least 99%, or 100% identity to SEQ ID NO: 6. In some aspects, the miRNA-15a target sequence comprises the nucleic acid sequence of SEQ ID NO: 6. In some aspects, the miRNA that is expressed in inner ear hair cells is miR-182. In some aspects, the miRNA-182 target sequence comprises the nucleic acid sequence with at least 85%, at least 90%, at least 95%, at least 96%, at least 97%, at least 98%, at least 99%, or 100% identity to SEQ ID NO: 78. In some aspects, the miRNA-182 target sequence comprises the nucleic acid sequence of SEQ ID NO: 78. In some aspects, the miRNA that is expressed in inner ear hair cells is miR-183. In some aspects, the miRNA-183 target sequence comprises the nucleic acid sequence with at least 85%, at least 90%, at least 95%, at least 96%, at least 97%, at least 98%, at least 99%, or 100% identity to SEQ ID NO: 79. In some aspects, the miRNA-183 target sequence comprises the nucleic acid sequence of SEQ ID NO: 79.

In some aspects, the miRTS may be a target sequence for a miRNA that is expressed in spiral ganglion cells. In some aspects, the miRNA that is expressed in spiral ganglion cells reduces, suppresses, inhibits, or eliminates expression of the polypeptide (e.g., a therapeutic polypeptide) in the spiral ganglion cells. In some aspects, miRNAs that are expressed in the spiral ganglion cells are miR-194, miR-18a, miR-99a, miR-30b, miR-15a, miR182, or miR-183. In some aspects, the miRNA that is expressed in ear hair cells is miR-194. In some aspects, the miRNA-194 target sequence comprises the nucleic acid sequence with at least 85%, at least 90%, at least 95%, at least 96%, at least 97%, at least 98%, at least 99%, or 100% identity to SEQ ID NO: 1. In some aspects, the miRNA-194 target sequence comprises the nucleic acid sequence of SEQ ID NO: 1. In some aspects, the miRNA that is expressed in ear hair cells is miR-18a. In some aspects, the miRNA-18a target sequence comprises the nucleic acid sequence with at least 85%, at least 90%, at least 95%, at least 96%, at least 97%, at least 98%, at least 99%, or 100% identity to SEQ ID NO: 3. In some aspects, the miRNA-18a target sequence comprises the nucleic acid sequence of SEQ ID NO: 3. In some aspects, the miRNA that is expressed in ear hair cells is miR-99a. In some aspects, the miRNA-99a target sequence comprises the nucleic acid sequence with at least 85%, at least 90%, at least 95%, at least 96%, at least 97%, at least 98%, at least 99%, or 100% identity to SEQ ID NO: 4. In some aspects, the miRNA-99a target sequence comprises the nucleic acid sequence of SEQ ID NO: 4. In some aspects, the miRNA that is expressed in ear hair cells is miR-30b. In some aspects, the miRNA-30b target sequence comprises the nucleic acid sequence with at least 85%, at least 90%, at least 95%, at least 96%, at least 97%, at least 98%, at least 99%, or 100% identity to SEQ ID NO: 5. In some aspects, the miRNA-30b target sequence comprises the nucleic acid sequence of SEQ ID NO: 5. In some aspects, the miRNA that is expressed in ear hair cells is miR-15a. In some aspects, the miRNA-15a target sequence comprises the nucleic acid sequence with at least 85%, at least 90%, at least 95%, at least 96%, at least 97%, at least 98%, at least 99%, or 100% identity to SEQ ID NO: 6. In some aspects, the miRNA-15a target sequence comprises the nucleic acid sequence of SEQ ID NO: 6. In some aspects, the miRNA that is expressed in ear hair cells is miR-182. In some aspects, the miRNA-182 target sequence comprises the nucleic acid sequence with at least 85%, at least 90%, at least 95%, at least 96%, at least 97%, at least 98%, at least 99%, or 100% identity to SEQ ID NO: 78. In some aspects, the miRNA-182 target sequence comprises the nucleic acid sequence of SEQ ID NO: 78. In some aspects, the miRNA that is expressed in ear hair cells is miR-183. In some aspects, the miRNA-183 target sequence comprises the nucleic acid sequence with at least 85%, at least 90%, at least 95%, at least 96%, at least 97%, at least 98%, at least 99%, or 100% identity to SEQ ID NO: 79. In some aspects, the miRNA-183 target sequence comprises the nucleic acid sequence of SEQ ID NO: 79.

In some aspects, the miRTS may be a target sequence for a miRNA that is expressed in basilar membrane cells. In some aspects, the miRNA that is expressed in basilar membrane cells reduces, suppresses, inhibits, or eliminates expression of the polypeptide (e.g., a therapeutic polypeptide) in the basilar membrane cells. In some aspects, miRNAs that are expressed in basilar membrane cells are miR-99a, miR-30b, and miR-15a. In some aspects, the miRNA that is expressed in ear hair cells is miR-99a. In some aspects, the miRNA-99a target sequence comprises the nucleic acid sequence with at least 85%, at least 90%, at least 95%, at least 96%, at least 97%, at least 98%, at least 99%, or 100% identity to SEQ ID NO: 4. In some aspects, the miRNA-99a target sequence comprises the nucleic acid sequence of SEQ ID NO: 4. In some aspects, the miRNA that is expressed in ear hair cells is miR-30b. In some aspects, the miRNA-30b target sequence comprises the nucleic acid sequence with at least 85%, at least 90%, at least 95%, at least 96%, at least 97%, at least 98%, at least 99%, or 100% identity to SEQ ID NO: 5. In some aspects, the miRNA-30b target sequence comprises the nucleic acid sequence of SEQ ID NO: 5. In some aspects, the miRNA that is expressed in ear hair cells is miR-15a. In some aspects, the miRNA-15a target sequence comprises the nucleic acid sequence with at least 85%, at least 90%, at least 95%, at least 96%, at least 97%, at least 98%, at least 99%, or 100% identity to SEQ ID NO: 6. In some aspects, the miRNA-15a target sequence comprises the nucleic acid sequence of SEQ ID NO: 6.

In some aspects, the miRTS may be a target sequence for a miRNA that is expressed in lateral supporting cells. In some aspects, the miRNA that is expressed in lateral supporting cells reduces, suppresses, inhibits, or eliminates expression of the polypeptide (e.g., a therapeutic polypeptide) in the lateral supporting cells. In some aspects, miRNAs that are expressed in lateral supporting cells are miR-99a, miR-30b, and miR-15a. In some aspects, the miRNA that is expressed in ear hair cells is miR-99a. In some aspects, the miRNA-99a target sequence comprises the nucleic acid sequence with at least 85%, at least 90%, at least 95%, at least 96%, at least 97%, at least 98%, at least 99%, or 100% identity to SEQ ID NO: 4. In some aspects, the miRNA-99a target sequence comprises the nucleic acid sequence of SEQ ID NO: 4. In some aspects, the miRNA that is expressed in ear hair cells is miR-30b. In some aspects, the miRNA-30b target sequence comprises the nucleic acid sequence with at least 85%, at least 90%, at least 95%, at least 96%, at least 97%, at least 98%, at least 99%, or 100% identity to SEQ ID NO: 5. In some aspects, the miRNA-30b target sequence comprises the nucleic acid sequence of SEQ ID NO: 5. In some aspects, the miRNA that is expressed in ear hair cells is miR-15a. In some aspects, the miRNA-15a target sequence comprises the nucleic acid sequence with at least 85%, at least 90%, at least 95%, at least 96%, at least 97%, at least 98%, at least 99%, or 100% identity to SEQ ID NO: 6. In some aspects, the miRNA-15a target sequence comprises the nucleic acid sequence of SEQ ID NO: 6.

In some aspects, the miRTS may be a target sequence for a miRNA that is expressed in medial supporting cells. In some aspects, the miRNA that is expressed in medial supporting cells reduces, suppresses, inhibits, or eliminates expression of the polypeptide (e.g., a therapeutic polypeptide) in the medial supporting cells. In some aspects, miRNAs that are expressed in medial supporting cells are miR182 and miR-183. In some aspects, the miRNA that is expressed in ear hair cells is miR-182. In some aspects, the miRNA-182 target sequence comprises the nucleic acid sequence with at least 85%, at least 90%, at least 95%, at least 96%, at least 97%, at least 98%, at least 99%, or 100% identity to SEQ ID NO: 78. In some aspects, the miRNA-182 target sequence comprises the nucleic acid sequence of SEQ ID NO: 78. In some aspects, the miRNA that is expressed in ear hair cells is miR-183. In some aspects, the miRNA-183 target sequence comprises the nucleic acid sequence with at least 85%, at least 90%, at least 95%, at least 96%, at least 97%, at least 98%, at least 99%, or 100% identity to SEQ ID NO: 79. In some aspects, the miRNA-183 target sequence comprises the nucleic acid sequence of SEQ ID NO: 79.

In some aspects, the miRTS may be a target sequence for a miRNA that is expressed in spiral limbus cells. In some aspects, the miRNA that is expressed in spiral limbus cells reduces, suppresses, inhibits, or eliminates expression of the polypeptide (e.g., a therapeutic polypeptide) in the spiral limbus cells. In some aspects, miRNAs that are expressed in spiral limbus cells are miR182 and miR-183. In some aspects, the miRNA that is expressed in ear hair cells is miR-182. In some aspects, the miRNA-182 target sequence comprises the nucleic acid sequence with at least 85%, at least 90%, at least 95%, at least 96%, at least 97%, at least 98%, at least 99%, or 100% identity to SEQ ID NO: 78. In some aspects, the miRNA-182 target sequence comprises the nucleic acid sequence of SEQ ID NO: 78. In some aspects, the miRNA that is expressed in ear hair cells is miR-183. In some aspects, the miRNA-183 target sequence comprises the nucleic acid sequence with at least 85%, at least 90%, at least 95%, at least 96%, at least 97%, at least 98%, at least 99%, or 100% identity to SEQ ID NO: 79. In some aspects, the miRNA-183 target sequence comprises the nucleic acid sequence of SEQ ID NO: 79.

In some aspects, a non-endogenous regulatory region included in a UTR may comprise multiple miRNA regulatory target sites (miRTS). In some aspects, a UTR may comprise at least one miRNA-182 target site and at least one miRNA-183 target site. In some aspects, a non-endogenous regulatory region included in a UTR is a destabilizing domain, and is exemplified by SEQ ID NO: 80. In some aspects, a UTR may include a sequence that is at least 85%, at least 90%, at least 95%, at least 96%, at least 97%, at least 98% at least 99%, or 100% identical to a non-endogenous regulatory region exemplified by SEQ ID NO: 80.

miRNA-182 target Sequence
(SEQ ID NO: 78)
AGTGTGAGTTCTACCATTGCCAAA
miRNA-183 target Sequence
(SEQ ID NO: 79)
AGTGAATTCTACCAGTGCCATA
miRNA-194 target Sequence
(SEQ ID NO: 1)
TCCACATGGAGTTGCTGTTACA
miRNA-140 target Sequence
(SEQ ID NO: 2)
CCGTGGTTCTACCCTGTGGTA
miRNA-18a target Sequence
(SEQ ID NO: 3)
CTATCTGCACTAGATGCACCTTA
miRNA-99a target Sequence
(SEQ ID NO: 4)
CACAAGATCGGATCTACGGGTT
miRNA-30b target Sequence
(SEQ ID NO: 5)
CTGAGTGTAGGATGTTTACA
miRNA-15a target Sequence
(SEQ ID NO: 6)
CACAAACCATTATGTGCTGCTA
Exemplary mRNA destabilizing domain Sequence
(SEQ ID NO: 80)
GAGCTCAGTGTGAGTTCTACCATTGCCAAACTCGAGCAGTGAATT
CTACCAGTGCCATAGGATCCAGTGTGAGTTCTACCATTGCCAAAG
GTACCCAGTGAATTCTACCAGTGCCATAGTTAAC

TABLE 3
Exemplary microRNA regulatory target sites

| microRNA regulatory target sites | SEQ ID NO |
|---|---|
| miRNA-182 target sequence | 78 |
| miRNA-183 target sequence | 79 |
| miRNA-194 target sequence | 1 |
| miRNA-140 target sequence | 2 |
| miRNA-18a target sequence | 3 |
| miRNA-99a target sequence | 4 |
| miRNA-30b target sequence | 5 |
| miRNA-15a target sequence | 6 |

### Internal Ribosome Entry Sites (IRES)

In some aspects, a construct encoding a polypeptide (e.g., a therapeutic polypeptide) can include an internal ribosome entry site (IRES). An IRES forms a complex secondary structure that allows translation initiation to occur from any position with an mRNA immediately downstream from where the IRES is located (see, e.g., Pelletier and Sonenberg, Mol. Cell. Biol. 8(3):1103-1112, 1988).

There are several IRES sequences known to those in skilled in the art, including those from, e.g., foot and mouth disease virus (FMDV), encephalomyocarditis virus (EMCV), human rhinovirus (HRV), cricket paralysis virus, human immunodeficiency virus (HIV), hepatitis A virus (HAV), hepatitis C virus (HCV), and poliovirus (PV). See e.g., Alberts, Molecular Biology of the Cell, Garland Science, 2002; and Hellen et al., Genes Dev. 15(13):1593-612, 2001, each of which is incorporated in its entirety herein by reference.

In some aspects, the IRES sequence that is incorporated into a construct that encodes a polypeptide (e.g., a therapeutic polypeptide) is the foot and mouth disease virus (FMDV) 2A sequence. The Foot and Mouth Disease Virus 2A sequence is a small peptide (approximately 18 amino acids in length) that has been shown to mediate the cleavage of polyproteins (Ryan, M D et al., EMBO 4:928-933, 1994; Mattion et al., J Virology 70:8124-8127, 1996; Furler et al., Gene Therapy 8:864-873, 2001; and Halpin et al., Plant Journal 4:453-459, 1999, each of which is incorporated in its entirety herein by reference). The cleavage activity of the 2A sequence has previously been demonstrated in artificial systems including plasmids and gene therapy constructs (AAV and retroviruses) (Ryan et al., EMBO 4:928-933, 1994; Mattion et al., J Virology 70:8124-8127, 1996; Furler et al., Gene Therapy 8:864-873, 2001; and Halpin et al., Plant Journal 4:453-459, 1999; de Felipe et al., Gene Therapy 6:198-208, 1999; de Felipe et al., Human Gene Therapy I I: 1921-1931, 2000; and Klump et al., Gene Therapy 8:811-817, 2001, each of which is incorporated in its entirety herein by reference).

### Splice Sites

In some aspects, any of the constructs provided herein can include splice donor and/or splice acceptor sequences, which are functional during RNA processing occurring during transcription. In some aspects, splice sites are involved in trans-splicing.

Exemplary splice donor intron
(SEQ ID NO: SEQ ID NO: 23)
GTAAGTATCAAGGTTACAAGACAGGTTTAAGGAGACCAATAGAAA
CTGGGCTTGTCGAGACAGAGAAGACTCTTGCGTTTCT
Exemplary splice acceptor intron
(SEQ ID NO: SEQ ID NO: 24)
GATAGGCACCTATTGGTCTTACTGACATCCACTTTGCCTTTCTCT
CCACAG

### Polyadenylation Sequences

In some aspects, a construct provided herein can include a polyadenylation (poly(A)) signal sequence. Most nascent eukaryotic mRNAs possess a poly(A) tail at their 3′ end, which is added during a complex process that includes cleavage of the primary transcript and a coupled polyadenylation reaction driven by the poly(A) signal sequence (see, e.g., Proudfoot et al., Cell 108:501-512, 2002, which is incorporated herein by reference in its entirety). A poly(A) tail confers mRNA stability and transferability (Molecular Biology of the Cell, Third Edition by B. Alberts et al., Garland Publishing, 1994, which is incorporated herein by reference in its entirety). In some aspects, a poly(A) signal sequence is positioned 3′ to the coding sequence.

As used herein, “polyadenylation” refers to the covalent linkage of a polyadenylyl moiety, or its modified variant, to a messenger RNA molecule. In eukaryotic organisms, most messenger RNA (mRNA) molecules are polyadenylated at the 3′ end. A 3′ poly(A) tail is a long sequence of adenine nucleotides (e.g., 50, 60, 70, 100, 200, 500, 1000, 2000, 3000, 4000, or 5000) added to the pre-mRNA through the action of an enzyme, polyadenylate polymerase. In some aspects, a poly(A) tail is added onto transcripts that contain a specific sequence, e.g., a polyadenylation (or poly(A)) signal. A poly(A) tail and associated proteins aid in protecting mRNA from degradation by exonucleases. Polyadenylation also plays a role in transcription termination, export of the mRNA from the nucleus, and translation. Polyadenylation typically occurs in the nucleus immediately after transcription of DNA into RNA, but also can occur later in the cytoplasm. After transcription has been terminated, an mRNA chain is cleaved through the action of an endonuclease complex associated with RNA polymerase. A cleavage site is usually characterized by the presence of the base sequence AAUAAA near the cleavage site. After the mRNA has been cleaved, adenosine residues are added to the free 3′ end at the cleavage site.

As used herein, a “poly(A) signal sequence” or “polyadenylation signal sequence” is a sequence that triggers the endonuclease cleavage of an mRNA and the addition of a series of adenosines to the 3′ end of the cleaved mRNA.

There are several poly(A) signal sequences that can be used, including those derived from bovine growth hormone (bGH) (Woychik et al., Proc. Natl. Acad Sci. US.A. 81(13):3944-3948, 1984; U.S. Pat. No. 5,122,458, each of which is incorporated herein by reference in its entirety), mouse-β-globin, mouse-α-globin (Orkin et al., EMBO J 4(2):453-456, 1985; Thein et al., Blood71(2):313-319, 1988, each of which is incorporated herein by reference in its entirety), human collagen, polyoma virus (Batt et al., Mol. Cell Biol. 15(9):4783-4790, 1995, which is incorporated herein by reference in its entirety), the Herpes simplex virus thymidine kinase gene (HSV TK), IgG heavy-chain gene polyadenylation signal (US 2006/0040354, which is incorporated herein by reference in its entirety), human growth hormone (hGH) (Szymanski et al., Mol. Therapy 15(7):1340-1347, 2007, which is incorporated herein by reference in its entirety), the group comprising a SV40 poly(A) site, such as the SV40 late and early poly(A) site (Schek et al., Mol. Cell Biol. 12(12):5386-5393, 1992, which is incorporated herein by reference in its entirety).

The poly(A) signal sequence can be AATAAA. The AATAAA sequence may be substituted with other hexanucleotide sequences with homology to AATAAA and that are capable of signaling polyadenylation, including ATTAAA, AGTAAA, CATAAA, TATAAA, GATAAA, ACTAAA, AATATA, AAGAAA, AATAAT, AAAAAA, AATGAA, AATCAA, AACAAA, AATCAA, AATAAC, AATAGA, AATTAA, or AATAAG (see, e.g., WO 06/12414, which is incorporated herein by reference in its entirety).

In some aspects, a poly(A) signal sequence can be a synthetic polyadenylation site (see, e.g., the pCl-neo expression construct of Promega that is based on Levitt el al., Genes Dev. 3(7):1019-1025, 1989, which is incorporated herein by reference in its entirety). In some aspects, a poly(A) signal sequence is the polyadenylation signal of soluble neuropilin-1 (sNRP) (AAATAAAATACGAAATG; SEQ ID NO: 89) (see, e.g., WO 05/073384, which is incorporated herein by reference in its entirety). In some aspects, a poly(A) signal sequence comprises or consists of the SV40 poly(A) site. In some aspects, a poly(A) signal comprises or consists of SEQ ID NO: 25. In some aspects, a poly(A) signal sequence comprises or consists of bGHpA. In some aspects, a poly(A) signal comprises or consists of SEQ ID NO: 26. Additional examples of poly(A) signal sequences are known in the art. In some aspects, a poly(A) sequence is at least 85%, at least 90%, at least 95%, at least 96%, at least 97%, at least 98% at least 99%, or 100% identical to the poly(A) sequence represented by SEQ ID NO: 25.

Exemplary bGH poly(A) signal Sequence
(SEQ ID NO: 25)
CTGTGCCTTCTAGTTGCCAGCCATCTGTTGTTTGCCCCTCCCCCG
TGCCTTCCTTGACCCTGGAAGGTGCCACTCCCACTGTCCTTTCCT
AATAAAATGAGGAAATTGCATCGCATTGTCTGAGTAGGTGTCATT
CTATTCTGGGGGGTGGGGTGGGGCAGGACAGCAAGGGGGAGGATT
GGGAAGACAATAGCAGGCATGCTGGGGATGCGGTGGGCTCTATGG
Exemplary SV40 poly(A) signal Sequence
(SEQ ID NO: 26)
AACTTGTTTATTGCAGCTTATAATGGTTACAAATAAAGCAATAGC
ATCACAAATTTCACAAATAAAGCATTTTTTTCACTGCATTCTAGT
TGTGGTTTGTCCAAACTCATCAATGTATCTTA

### Additional Sequences

In some aspects, constructs of the present disclosure may include one or more filler sequences. In some aspects, filler sequences may function as regulatory elements, altering construct expression. In some such aspects, filler sequences may not be fully removed prior to manufacturing for administration to a subject. In some aspects, filler sequences may have functional roles including as linker sequences, as regulatory regions, or as stabilizing regions. As will be appreciated by those skilled in the art, filler sequences may vary significantly in primary sequence while retaining their desired function. In some aspects, constructs may contain any combination of filler sequences, exemplary filler sequences which may function as regulatory sequences are represented by SEQ ID NO: 117, or 118.

In some aspects, constructs of the present disclosure may comprise a T2A element or sequence. In some aspects, constructs of the present disclosure may include one or more cloning sites. In some such aspects, cloning sites may not be fully removed prior to manufacturing for administration to a subject. In some aspects, cloning sites may have functional roles including as linker sequences, portions of a Kozak site, or as sites encoding a stop codon. As will be appreciated by those skilled in the art, cloning sites may vary significantly in primary sequence while retaining their desired function. In some aspects, constructs may contain any combination of cloning sites, exemplary cloning sites are represented by SEQ ID NO: 29, 30, 31, 32, 33, 34, 35, 36, 37, or 85. In some aspects, constructs may contain additional cloning sites less than five nucleotides in length.

Exemplary Regulatory sequence C3
(SEQ ID NO: 117)
CTTCTTCTGGAGTCTTTTCTGGAATAATTCTGGGAGTGGGCTCAG
CCTGCGGGAGAGTAACATTTTTATAACTTGATAGATGTAGCTGAG
ATGCCTCCCAGAGGGGAGACCCGCCTCTCCTCCGGCAGCTGTGCA
CGTAGGCTTGTTCCCAGCAGCCTGGCCAGGGTGGTCCACCTGGTG
TTTCTCATCTTCTTTCCCCGGAGCGCTGACTCCTGCGCGTCCTCT
TGGAAGACTCTTGACAGGACGGGTGTTTTATGGGTGTGATTCAGT
GTCCTCTTGCATCAGTTCAATGTGGTGGTGTTCAATCAACCCTTG
TAGCGTTAGCAAAATTTGCTCAAGTCATTCCGCAGGAATGTCTGT
GTCTTGCTTCCAAGAAAGCTTGTAAGTGCCGGCAACAGGCCAAGC
AGCTCACAAACCTGACCACAAGCCTGTGAGTAATTGTGGGGCAGC
ACTTAGCAGTCTTTTATTTTCGACTTATTAAAGTCTCATCTTGGC
CTCACCTTCTCCCTGGAAGGTGGCGTGGGTGGGAACCACTGGGTC
AGATCTTTTTCACCCTTGCCGTGGAGCCAGTTTCCTGTTGCATGT
GGGGGAAGCAACATGTGGTGAAGAGTATAGAAAACGAAAACATGT
GGGTACAGTATGTATAAGTGGAGGGAACAAACTCATAATTCCAAC
TAGTTTCTCATGAGAGACTCATGAATCATTGTGGTAGTTCTCAAT
ATAAACTTAATCTAGGCCGGATGTGGTGGCTCACACCTGTAATCT
CAGCACTCTGGGTGGATCACTTGAGGTCAGGAGTTTGAGACCAGT
CTGACCAACATGGAGAAACCCCATCGCTACTAAAAATACAAAATT
ATCCAGATGTGGTGGCTCACACCTGTAATCCCAGCACTTTGGGAG
GCTGAGGCGGGTGGATCACTTGAGGTCAGGAGTTTGAGACCAGCC
TGACCAACATGGAGAAACTGTGTCTCTACTAAAAATACAAAATTA
GCTGGGCGTGGTGACGCATGCCTGTAATCCCAGCTATTTGGAGGC
CGAAGCAGG
Exemplary Regulatory sequence D7
(SEQ ID NO: 118)
CTTCTTCTGGAGTCTTTTCTGGAATAATTCTGGGAGTGGGCTCAG
CCTGCGGGAGAGTAACATTTTTATAACTTGATAGATGTAGCTGAG
ATGCCTCCCAGAGGGGAGACCCGCCTCTCCTCCGGCAGCTGTGCA
CGTAGGCTTGTTCCCAGCAGCCTGGCCAGGGTGGTCCACCTGGTG
TTTCTCATCTTCTTTCCCCGGAGCGCTGACTCCTGCGCGTCCTCT
TGGAAGACTCTTGACAGGACGGGTGTTTTATGGGTGTGATTCAGT
GTCCTCTTGCATCAGTTCAATGTGGTGGTGTTCAATCAACCCTTG
TAGCGTTAGCAAAATTTGCTCAAGTCATTCCGCAGGAATGTCTGT
GTCTTGCTTCCAAGAAAGCTTGTAAGTGCCGGCAACAGGCCAAGC
AGCTCACAAACCTGACCACAAGCCTGTGAGTAATTGTGGGGCAGC
ACTTAGCAGTCTTTTATTTTCGACTTATTAAAGTCTCATCTTGGC
CTCACCTTCTCCCTGGAAGGTGGCGTGGGTGGGAACCACTGGGTC
AGATCTTTTTCACCCTTGCCGTGGAGCCAGTTTCCTGTTGCATGT
GGGGGAAGCAACATGTGGTGAAGAGTATAGAAAACGAAAACATGT
GGGTACAGTATGTATAAGTGGAGGGAACAAACTCATAATTCCAAC
TAGTTTCTCATGAGAGACTCATGAATCATTGTGGTAGTTCTCAAT
ATAAACTTAATCTAGGCCGGATGTGGTGGCTCACACCTGTAATCT
CAGCACTCTGGGTGGATCACTTGAGGTCAGGAGTTTGAGACCAGT
CTGACCAACATGGAGAAACCCCATCGCTACTAAAAATACAAAATT
ATCCAGATGTGGTGGCTCACACCTGTAATCCCAGCACTTTGGGAG
GCTGAGGCGGGTGGATCACTTGAGGTCAGGAGTTTGAGACCAGCC
TGACCAACATGGAGAAACTGTGTCTCTACTAAAAATACAAAATTA
GCTGGGCGTGGTGACGCATGCCTGTAATCCCAGCTATTTGGAGGC
CGAAGCAGG
Exemplary cloning site A
(SEQ ID NO: 29)
TTGTCGACGCGGCCGCACGCGT
Exemplary cloning site B
(SEQ ID NO: 30)
CTCCTGGGCAACGTGCTGGTTATTGTGACCGGTGCCACC
Exemplary cloning site C
(SEQ ID NO: 31)
TAAGAGCTCGCTGATCAGCCTCGA
Exemplary cloning site D
(SEQ ID NO: 32)
AAGCTTGAATTCAGCTGACGTGCCTCGGACCGCCTAGG
Exemplary cloning site E
(SEQ ID NO: 33)
TAAGAGCTC
Exemplary cloning site F
(SEQ ID NO: 34)
GCTGATCAGCCTCGA
Exemplary cloning site G
(SEQ ID NO: 35)
GGCATTCCGGTACTGTTGGTAAAGCCACCAGCAAACCGC
CCAGAGTAGAAGACCGGTGGCCACC
Exemplary cloning site H
(SEQ ID NO: 36)
AAGCTTGAATTC
Exemplary cloning site I
(SEQ ID NO: 37)
AGCTGACGTGCCTCGGACCGCCTAGG
Exemplary cloning site J
(SEQ ID NO: 70)
GCGGCCGCACGCGT
Exemplary cloning site K
(SEQ ID NO: 71)
GCGGCCGCACGCGTGGT
Exemplary cloning site L
(SEQ ID NO: 72)
CTCCTGGGCAACGTGCTGGTTATTGTGACCGGT
Exemplary cloning site M
(SEQ ID NO: 73)
CGCTAGCCACC
Exemplary cloning site N
(SEQ ID NO: 74)
ACCGGTCGCTAGCCACC
Exemplary cloning site O
(SEQ ID NO: 75)
GAGCTCGCTGATCAGCCTCGA
Exemplary cloning site P
(SEQ ID NO: 76)
AAGCTTGAATTCAGCTGACGTGCCTCGGACCGCT
Exemplary cloning site Q
(SEQ ID NO: 85)
CTCACCGGT
Exemplary linker Sequence
(SEQ ID NO: 77)
GGATCCCGGGCT

### Reporter Sequences, Elements, or Reporter Polypeptides

In some aspects, constructs provided herein can optionally include a sequence encoding a reporter polypeptide and/or protein (“a reporter sequence”). Non-limiting examples of reporter sequences include DNA sequences encoding: a beta-lactamase, a beta-galactosidase (LacZ), an alkaline phosphatase, a thymidine kinase, a green fluorescent protein (GFP), a red fluorescent protein, an mCherry fluorescent protein, a yellow fluorescent protein, a chloramphenicol acetyltransferase (CAT), FLAG, and a luciferase. Additional examples of reporter sequences are known in the art. Non-limiting examples of reporter polypeptides include a beta-lactamase, a beta-galactosidase (LacZ), an alkaline phosphatase, a thymidine kinase, a green fluorescent protein (GFP), a red fluorescent protein, an mCherry fluorescent protein, a yellow fluorescent protein, a chloramphenicol acetyltransferase (CAT), FLAG, and a luciferase. When associated with control elements which drive their expression, the reporter sequence can provide signals detectable by conventional means, including enzymatic, radiographic, colorimetric, fluorescence, or other spectrographic assays; fluorescent activating cell sorting (FACS) assays; immunological assays (e.g., enzyme linked immunosorbent assay (ELISA), radioimmunoassay (MA), and immunohistochemistry).

In some aspects, a reporter sequence is the LacZ gene, and the presence of a construct carrying the LacZ gene in a mammalian cell (e.g., a cochlear hair cell) is detected by assays for beta-galactosidase activity. When the reporter polypeptide is a fluorescent protein (e.g., green fluorescent protein) or luciferase, the presence of a construct carrying the fluorescent protein or luciferase in a mammalian cell (e.g., a cochlear hair cell) may be measured by fluorescent techniques (e.g., fluorescent microscopy or FACS) or light production in a luminometer (e.g., a spectrophotometer or an IVIS imaging instrument). In some aspects, a reporter sequence can be used to verify the tissue-specific targeting capabilities and tissue-specific promoter regulatory and/or control activity of any of the constructs described herein. In some aspects, a reporter polypeptide can be used to verify the tissue-specific targeting capabilities and tissue-specific promoter regulatory and/or control activity of any of the constructs described herein.

In some aspects, a reporter sequence is a FLAG tag (e.g., a 3xFLAG tag), and the presence of a construct carrying the FLAG tag in a mammalian cell (e.g., an inner ear cell, e.g., a cochlear hair or supporting cell) is detected by protein binding or detection assays (e.g., Western blots, immunohistochemistry, radioimmunoassay (MA), mass spectrometry). An exemplary 3xFLAG tag sequence is provided as SEQ ID NO: 42.

Exemplary 3xFLAG tag Sequence
(SEQ ID NO: 42)
GGATCCCGGGCTGACTACAAAGACCATGACGGTGATTATAAAGATC
ATGACATCGACTACAAGGATGACGATGACAAG
Exemplary 3xFLAG tag sequence with stop codon
(SEQ ID NO: 81)
GACTACAAAGACCATGACGGTGATTATAAAGATCATGACATCGACTA
CAAGGATGACGATGACAAGTAA
Exemplary barcode tag
(SEQ ID NO: 62)
GTGTCACC
Exemplary barcode tag
(SEQ ID NO: 55)
CACAACCT
Exemplary barcode tag
(SEQ ID NO: 27)
CGTGTGTT
Exemplary barcode tag
(SEQ ID NO: 41)
TCGTGGGT
Exemplary barcode tag
(SEQ ID NO: 39)
GCAAACTG
Exemplary barcode tag
(SEQ ID NO: 108)
CCTACGCT
Exemplary barcode tag
(SEQ ID NO: 109)
GCCAAAGC
Exemplary barcode tag
(SEQ ID NO: 110)
CCATCCAC
Exemplary barcode tag
(SEQ ID NO: 111)
CCCGTTCT
Exemplary barcode tag
(SEQ ID NO: 112)
TTCACTGG
Exemplary barcode tag
(SEQ ID NO: 113)
ATACTCTC
Exemplary barcode tag
(SEQ ID NO: 114)
GGCACTTC
Exemplary barcode tag
(SEQ ID NO: 115)
TTTCAGGT

### AAV Capsids

The present disclosure provides one or more polynucleotide constructs packaged into an AAV capsid. In some aspects, an AAV capsid is from or derived from an AAV capsid of an AAV2, 3, 4, 5, 6, 7, 8, 9, 10, rh8, rh10, rh39, rh43, AAV2-tYF, AAV2-P2V2, AAV2-P2V3, AAV2-MeStYFTV, AAV2-MeB, AAV2-P2V6, AAV2-DGEDF, or Anc80 serotype, or one or more hybrids thereof. In some aspects, an AAV capsid is from an AAV ancestral serotype. In some aspects, an AAV capsid is an ancestral (Anc) AAV capsid. An Anc capsid is created from a construct sequence that is constructed using evolutionary probabilities and evolutionary modeling to determine a probable ancestral sequence. Thus, an Anc capsid/construct sequence is not known to have existed in nature. For example, in some aspects, an AAV capsid is an Anc80 capsid (e.g., an Anc80L65 capsid). In some aspects, an AAV capsid is created using a template nucleotide coding sequence comprising SEQ ID NO: 43. In some aspects, the capsid comprises a polypeptide represented by SEQ ID NO: 44. In some aspects, the capsid comprises a polypeptide with at least 85%, at least 90%, at least 95%, at least 96%, at least 97%, at least 98% at least 99%, or 100% identical to the polypeptide represented by SEQ ID NO: 44.

As provided herein, any combination of AAV capsids and AAV constructs (e.g., comprising AAV ITRs) may be used in recombinant AAV (rAAV) particles of the present disclosure. For example, wild-type or variant AAV2 ITRs and Anc80 capsid (e.g., an Anc80L65 capsid), wild-type or variant AAV2 ITRs and AAV6 capsid, etc. In some aspects of the present disclosure, an AAV particle is wholly comprised of AAV2 components (e.g., capsid and ITRs are AAV2 serotype). In some aspects, an AAV particle is an AAV2/6, AAV2/8 or AAV2/9 particle (e.g., an AAV6, AAV8 or AAV9 capsid with an AAV construct having AAV2 ITRs). In some aspects of the present disclosure, an AAV particle is an AAV2/Anc80 particle that comprises an Anc80 capsid (e.g., comprising a polypeptide of SEQ ID NO: 44) that encapsidates an AAV construct with AAV2 ITRs (e.g., SEQ ID NOs: 8 and 9) flanking a portion of a coding sequence, for example, a nucleic acid encoding a polypeptide (e.g., a therapeutic polypeptide). Other AAV particles are known in the art and are described in, e.g., Sharma et al., Brain Res Bull. 2010 Feb. 15; 81(2-3): 273, which is incorporated in its entirety herein by reference. In some aspects, a capsid sequence is at least 85%, at least 90%, at least 95%, at least 96%, at least 97%, at least 98% at least 99%, or 100% identical to a capsid nucleotide or amino acid sequence represented by SEQ ID NO: 43 or 44, respectively.

Exemplary AAV Anc80 Capsid DNA Sequence
(SEQ ID NO: 43)
ATGGCTGCCGATGGTTATCTTCCAGATTGGCTCGAGGACAACCTC
TCTGAGGGCATTCGCGAGTGGTGGGACTTGAAACCTGGAGCCCCG
AAACCCAAAGCCAACCAGCAAAAGCAGGACGACGGCCGGGGTCTG
GTGCTTCCTGGCTACAAGTACCTCGGACCCTTCAACGGACTCGAC
AAGGGGGAGCCCGTCAACGCGGCGGACGCAGCGGCCCTCGAGCAC
GACAAGGCCTACGACCAGCAGCTCAAAGCGGGTGACAATCCGTAC
CTGCGGTATAACCACGCCGACGCCGAGTTTCAGGAGCGTCTGCAA
GAAGATACGTCTTTTGGGGGCAACCTCGGGCGAGCAGTCTTCCAG
GCCAAGAAGCGGGTTCTCGAACCTCTCGGTCTGGTTGAGGAAGGC
GCTAAGACGGCTCCTGGAAAGAAGAGACCGGTAGAGCAATCACCC
CAGGAACCAGACTCCTCTTCGGGCATCGGCAAGAAAGGCCAGCAG
CCCGCGAAGAAGAGACTCAACTTTGGGCAGACAGGCGACTCAGAG
TCAGTGCCCGACCCTCAACCACTCGGAGAACCCCCCGCAGCCCCC
TCTGGTGTGGGATCTAATACAATGGCAGCAGGCGGTGGCGCTCCA
ATGGCAGACAATAACGAAGGCGCCGACGGAGTGGGTAACGCCTCA
GGAAATTGGCATTGCGATTCCACATGGCTGGGCGACAGAGTCATC
ACCACCAGCACCCGAACCTGGGCCCTCCCCACCTACAACAACCAC
CTCTACAAGCAAATCTCCAGCCAATCGGGAGCAAGCACCAACGAC
AACACCTACTTCGGCTACAGCACCCCCTGGGGGTATTTTGACTTT
AACAGATTCCACTGCCACTTCTCACCACGTGACTGGCAGCGACTC
ATCAACAACAACTGGGGATTCCGGCCCAAGAGACTCAACTTCAAG
CTCTTCAACATCCAGGTCAAGGAGGTCACGACGAATGATGGCACC
ACGACCATCGCCAATAACCTTACCAGCACGGTTCAGGTCTTTACG
GACTCGGAATACCAGCTCCCGTACGTCCTCGGCTCTGCGCACCAG
GGCTGCCTGCCTCCGTTCCCGGCGGACGTCTTCATGATTCCTCAG
TACGGGTACCTGACTCTGAACAATGGCAGTCAGGCCGTGGGCCGT
TCCTCCTTCTACTGCCTGGAGTACTTTCCTTCTCAAATGCTGAGA
ACGGGCAACAACTTTGAGTTCAGCTACACGTTTGAGGACGTGCCT
TTTCACAGCAGCTACGCGCACAGCCAAAGCCTGGACCGGCTGATG
AACCCCCTCATCGACCAGTACCTGTACTACCTGTCTCGGACTCAG
ACCACGAGTGGTACCGCAGGAAATCGGACGTTGCAATTTTCTCAG
GCCGGGCCTAGTAGCATGGCGAATCAGGCCAAAAACTGGCTACCC
GGGCCCTGCTACCGGCAGCAACGCGTCTCCAAGACAGCGAATCAA
AATAACAACAGCAACTTTGCCTGGACCGGTGCCACCAAGTATCAT
CTGAATGGCAGAGACTCTCTGGTAAATCCCGGTCCCGCTATGGCA
ACCCACAAGGACGACGAAGACAAATTTTTTCCGATGAGCGGAGTC
TTAATATTTGGGAAACAGGGAGCTGGAAATAGCAACGTGGACCTT
GACAACGTTATGATAACCAGTGAGGAAGAAATTAAAACCACCAAC
CCAGTGGCCACAGAACAGTACGGCACGGTGGCCACTAACCTGCAA
TCGTCAAACACCGCTCCTGCTACAGGGACCGTCAACAGTCAAGGA
GCCTTACCTGGCATGGTCTGGCAGAACCGGGACGTGTACCTGCAG
GGTCCTATCTGGGCCAAGATTCCTCACACGGACGGACACTTTCAT
CCCTCGCCGCTGATGGGAGGCTTTGGACTGAAACACCCGCCTCCT
CAGATCCTGATTAAGAATACACCTGTTCCCGCGAATCCTCCAACT
ACCTTCAGTCCAGCTAAGTTTGCGTCGTTCATCACGCAGTACAGC
ACCGGACAGGTCAGCGTGGAAATTGAATGGGAGCTGCAGAAAGAA
AACAGCAAACGCTGGAACCCAGAGATTCAATACACTTCCAACTAC
AACAAATCTACAAATGTGGACTTTGCTGTTGACACAAATGGCGTT
TATTCTGAGCCTCGCCCCATCGGCACCCGTTACCTCACCCGTAAT
CTG
Exemplary AAV Anc80 Capsid Amino Acid Sequence
(SEQ ID NO: 44)
MAADGYLPDWLEDNLSEGIREWWDLKPGAPKPKANQQKQDDGRGL
VLPGYKYLGPFNGLDKGEPVNAADAAALEHDKAYDQQLKAGDNPY
LRYNHADAEFQERLQEDTSFGGNLGRAVFQAKKRVLEPLGLVEEG
AKTAPGKKRPVEQSPQEPDSSSGIGKKGQQPAKKRLNFGQTGDSE
SVPDPQPLGEPPAAPSGVGSNTMAAGGGAPMADNNEGADGVGNAS
GNWHCDSTWLGDRVITTSTRTWALPTYNNHLYKQISSQSGASTND
NTYFGYSTPWGYFDFNRFHCHFSPRDWQRLINNNWGFRPKRLNFK
LFNIQVKEVTTNDGTTTIANNLTSTVQVFTDSEYQLPYVLGSAHQ
GCLPPFPADVFMIPQYGYLTLNNGSQAVGRSSFYCLEYFPSQMLR
TGNNFEFSYTFEDVPFHSSYAHSQSLDRLMNPLIDQYLYYLSRTQ
TTSGTAGNRTLQFSQAGPSSMANQAKNWLPGPCYRQQRVSKTANQ
NNNSNFAWTGATKYHLNGRDSLVNPGPAMATHKDDEDKFFPMSGV
LIFGKQGAGNSNVDLDNVMITSEEEIKTTNPVATEQYGTVATNLQ
SSNTAPATGTVNSQGALPGMVWQNRDVYLQGPIWAKIPHTDGHFH
PSPLMGGFGLKHPPPQILIKNTPVPANPPTTFSPAKFASFITQYS
TGQVSVEIEWELQKENSKRWNPEIQYTSNYNKSTNVDFAVDTNGV
YSEPRPIGTRYLTRNL

### Compositions

Among other things, the present disclosure provides compositions. In some aspects, a composition comprises a construct as described herein. In some aspects, a composition comprises one or more constructs as described herein. In some aspects, a composition comprises a plurality of constructs as described herein. In some aspects, when more than one construct is included in the composition, the constructs are each different.

In some aspects, a composition comprises an AAV particle as described herein. In some aspects, a composition comprises one or more AAV particles as described herein. In some aspects, a composition comprises a plurality of AAV particles. In come aspects, when more than one AAV particle is included in the composition, the AAV particles are each different.

In some aspects, a composition comprises a vector as described herein.

In some aspects, a composition comprises a cell.

In some aspects, a composition is or comprises a pharmaceutical composition. In some aspects, the pharmaceutic composition comprises a pharmaceutically acceptable carrier. In some aspects, a composition is or comprises a synthetic perilymph solution. In some aspects, a synthetic perilymph solution comprises 20-200 mM NaCl; 1-5 mM KCl; 0.1-10 mM CaCl2; 1-10 mM glucose; and 2-50 mM HEPES, with a pH between about 6 and about 9.

### Dosing and Volume of Administration

In some aspects, a composition disclosed herein, e.g., one or a plurality of AAV vectors disclosed herein, is administered as a single dose or as a plurality of doses.

In some aspects, a composition disclosed herein is administered as a single dose. In some aspects, a composition disclosed herein is administered as a plurality of doses, e.g., 2, 3, 4, 5, 6, 7, 8, 9 or 10 doses.

In some aspects, a composition disclosed herein (e.g., a composition comprising one or a plurality of rAAV constructs disclosed herein) is administered at a volume of about 0.01 mL, about 0.02 mL, about 0.03 mL, about 0.04 mL, about 0.05 mL, about 0.06 mL, about 0.07 mL, about 0.08 mL, about 0.09 mL, about 1.00 mL, about 1.10 mL, about 1.20 mL, about 1.30 mL, about 1.40 mL, about 1.50 mL, about 1.60 mL, about 1.70 mL, about 1.80 mL, about 1.90 mL, or about 2.00 mL. In some aspects, a composition disclosed herein is administered at a volume of about 0.01 mL. In some aspects, a composition disclosed herein is administered at a volume of about 0.02 mL. In some aspects, a composition disclosed herein is administered at a volume of about 0.03 mL. In some aspects, a composition disclosed herein is administered at a volume of about 0.04 mL. In some aspects, a composition disclosed herein is administered at a volume of about 0.05 mL. In some aspects, a composition disclosed herein is administered at a volume of about 0.06 mL. In some aspects, a composition disclosed herein is administered at a volume of about 0.07 mL. In some aspects, a composition disclosed herein is administered at a volume of about 0.08 mL. In some aspects, a composition disclosed herein is administered at a volume of about 0.09 mL. In some aspects, a composition disclosed herein is administered at a volume of about 1.00 mL. In some aspects, a composition disclosed herein is administered at a volume of about 1.10 mL. In some aspects, a composition disclosed herein is administered at a volume of about 1.20 mL. In some aspects, a composition disclosed herein is administered at a volume of about 1.30 mL. In some aspects, a composition disclosed herein is administered at a volume of about 1.40 mL. In some aspects, a composition disclosed herein is administered at a volume of about 1.50 mL. In some aspects, a composition disclosed herein is administered at a volume of about 1.60 mL. In some aspects, a composition disclosed herein is administered at a volume of about 1.70 mL. In some aspects, a composition disclosed herein is administered at a volume of about 1.80 mL. In some aspects, a composition disclosed herein is administered at a volume of about 1.90 mL. In some aspects, a composition disclosed herein is administered at a volume of about 2.00 mL.

In some aspects, a composition disclosed herein (e.g., a composition comprising one or a plurality of rAAV constructs disclosed herein) is administered at a volume of about 0.01 to 2.00 mL, about 0.02 to 1.90 mL, about 0.03 to 1.8 mL, about 0.04 to 1.70 mL, about 0.05 to 1.60 mL, about 0.06 to 1.50 mL, about 0.06 to 1.40 mL, about 0.07 to 1.30 mL, about 0.08 to 1.20 mL, or about 0.09 to 1.10 mL. In some aspects a composition disclosed herein (e.g., a composition comprising one or a plurality of rAAV constructs disclosed herein) is administered at a volume of about 0.01 to 2.00 mL, about 0.02 to 2.00 mL, about 0.03 to 2.00 mL, about 0.04 to 2.00 mL, about 0.05 to 2.00 mL, about 0.06 to 2.00 mL, about 0.07 to 2.00 mL, about 0.08 to 2.00 mL, about 0.09 to 2.00 mL, about 0.01 to 1.90 mL, about 0.01 to 1.80 mL, about 0.01 to 1.70 mL, about 0.01 to 1.60 mL, about 0.01 to 1.50 mL, about 0.01 to 1.40 mL, about 0.01 to 1.30 mL, about 0.01 to 1.20 mL, about 0.01 to 1.10 mL, about 0.01 to 1.00 mL, about 0.01 to 0.09 mL.

In some aspects, a dosing regimen comprises delivery in a volume of at least 0.01 mL, at least 0.02 mL, at least 0.03 mL, at least 0.04 mL, at least 0.05 mL, at least 0.06 mL, at least 0.07 mL, at least 0.08 mL, at least 0.09 mL, at least 0.10 mL, at least 0.11 mL, at least 0.12 mL, at least 0.13 mL, at least 0.14 mL, at least 0.15 mL, at least 0.16 mL, at least 0.17 mL, at least 0.18 mL, at least 0.19 mL, or at least 0.20 mL per cochlea. In some aspects, a dosing regimen comprises delivery in a volume of at most 0.30 mL, at most 0.25 mL, at most 0.20 mL, at most 0.15 mL, at most 0.14 mL, at most 0.13 mL, at most 0.12 mL, at most 0.11 mL, at most 0.10 mL, at most 0.09 mL, at most 0.08 mL, at most 0.07 mL, at most 0.06 mL, or at most 0.05 mL per cochlea. In some aspects, the dosing regimen comprises delivery in a volume of about 0.05 mL, about 0.06 mL, about 0.07 mL, about 0.08 mL, about 0.09 mL, about 0.10 mL, about 0.11 mL, about 0.12 mL, about 0.13 mL, about 0.14 mL, or about 0.15 mL per cochlea, depending on the population.

### Single AAV Construct Compositions

In some aspects, the present disclosure provides compositions or systems comprising AAV particles comprised of a single construct. In some such aspects, a single construct may deliver a polynucleotide that encodes a functional (e.g., wild-type or otherwise functional, e.g., codon optimized) polypeptide (e.g., a therapeutic polypeptide). In some aspects, a construct is or comprises an rAAV construct. In some aspects described herein, a single rAAV construct is capable of expressing a polypeptide (e.g., a therapeutic polypeptide) thereof in a target cell (e.g., an inner ear supporting cell).

In some aspects, a single construct composition or system may comprise any or all of the exemplary construct components described herein. In some aspects, the, the construct comprises a nucleic acid sequence with at least 85%, at least 90%, at least 95%, at least 96%, at least 97%, at least 98% at least 99%, or 100% identity to SEQ ID NOs: 45-51, 82-84, 88, or 100-107. In some aspects, the, the construct comprises a nucleic acid sequence with at least 85%, at least 90%, at least 95%, at least 96%, at least 97%, at least 98% at least 99%, or 100% identity to SEQ ID NO: 82. In some aspects, an exemplary single construct is represented by SEQ ID NO: 82. In some aspects, the, the construct comprises a nucleic acid sequence with at least 85%, at least 90%, at least 95%, at least 96%, at least 97%, at least 98% at least 99%, or 100% identity to SEQ ID NO: 83. In some aspects, an exemplary single construct is represented by SEQ ID NO: 83. In some aspects, the, the construct comprises a nucleic acid sequence with at least 85%, at least 90%, at least 95%, at least 96%, at least 97%, at least 98% at least 99%, or 100% identity to SEQ ID NO: 84. In some aspects, an exemplary single construct is represented by SEQ ID NO: 84. In some aspects, the, the construct comprises a nucleic acid sequence with at least 85%, at least 90%, at least 95%, at least 96%, at least 97%, at least 98% at least 99%, or 100% identity to SEQ ID NO: 87. In some aspects, an exemplary single construct is represented by SEQ ID NO: 87.

In some aspects, the construct comprises the nucleic acid sequence of SEQ ID NO: 54. In some aspects, the, the construct comprises a nucleic acid sequence with at least 85%, at least 90%, at least 95%, at least 96%, at least 97%, at least 98% at least 99%, or 100% identity to SEQ ID NO: 54. In some aspects, the construct comprises the nucleic acid sequence of nucleotides 12-4754 of SEQ ID NO: 54. In some aspects, the, the construct comprises a nucleic acid sequence with at least 85%, at least 90%, at least 95%, at least 96%, at least 97%, at least 98% at least 99%, or 100% identity to 12-4754 of SEQ ID NO: 54.

In some aspects, the construct comprises the nucleic acid sequence of SEQ ID NO: 17. In some aspects, the, the construct comprises a nucleic acid sequence with at least 85%, at least 90%, at least 95%, at least 96%, at least 97%, at least 98% at least 99%, or 100% identity to SEQ ID NO: 17. In some aspects, the construct comprises nucleotides 12-4338 of SEQ ID NO: 17. In some aspects, the, the construct comprises a nucleic acid sequence with at least 85%, at least 90%, at least 95%, at least 96%, at least 97%, at least 98% at least 99%, or 100% identity to nucleotides 12-4338 of SEQ ID NO: 17.

In some aspects, the construct comprises the nucleic acid sequence of SEQ ID NO: 7. In some aspects, the, the construct comprises a nucleic acid sequence with at least 85%, at least 90%, at least 95%, at least 96%, at least 97%, at least 98% at least 99%, or 100% identity to SEQ ID NO: 7.

. In some aspects, the construct comprises nucleotides 12-4557 of SEQ ID NO: 7. In some aspects, the, the construct comprises a nucleic acid sequence with at least 85%, at least 90%, at least 95%, at least 96%, at least 97%, at least 98% at least 99%, or 100% identity nucleotides 12-4557 of SEQ ID NO: 7.

In some aspects, the construct comprises a nucleic acid sequence with at least 85%, at least 90%, at least 95%, at least 96%, at least 97%, at least 98% at least 99%, or 100% identity to SEQ ID NO: 61. In some aspects, the construct comprises the nucleic acid sequence of SEQ ID NO: 61. In some aspects, the construct comprises a nucleic acid sequence with at least 85%, at least 90%, at least 95%, at least 96%, at least 97%, at least 98% at least 99%, or 100% identity to nucleotides 12-4429 of SEQ ID NO: 61. In some aspects, the construct comprises the nucleic acid sequence of nucleotides 12-4429 of SEQ ID NO: 61.

In some aspects, the construct comprises a nucleic acid sequence with at least 85%, at least 90%, at least 95%, at least 96%, at least 97%, at least 98% at least 99%, or 100% identity to SEQ ID NO: 38. In some aspects, the construct comprises the nucleic acid sequence of SEQ ID NO: 38. In some aspects, the construct comprises a nucleic acid sequence with at least 85%, at least 90%, at least 95%, at least 96%, at least 97%, at least 98% at least 99%, or 100% identity to nucleotides 12-3976 of SEQ ID NO: 38. In some aspects, the construct comprises the nucleic acid sequence of nucleotides 12-3976 of SEQ ID NO: 38.

In some aspects, the construct comprises a nucleic acid sequence with at least 85%, at least 90%, at least 95%, at least 96%, at least 97%, at least 98% at least 99%, or 100% identity to SEQ ID NO: 100. In some aspects, the construct comprises SEQ ID NO: 100. In some aspects, the construct comprises a nucleic acid sequence with at least 85%, at least 90%, at least 95%, at least 96%, at least 97%, at least 98% at least 99%, or 100% identity to nucleotides 12-4645 of SEQ ID NO: 100. In some aspects, the construct comprises nucleotides 12-4645 of SEQ ID NO: 100.

In some aspects, the construct comprises a nucleic acid sequence with at least 85%, at least 90%, at least 95%, at least 96%, at least 97%, at least 98% at least 99%, or 100% identity to SEQ ID NO: 101. In some aspects, the construct comprises SEQ ID NO: 101. In some aspects, the construct comprises a nucleic acid sequence with at least 85%, at least 90%, at least 95%, at least 96%, at least 97%, at least 98% at least 99%, or 100% identity to nucleotides 12-4708 of SEQ ID NO: 101. In some aspects, the construct comprises nucleotides 12-4708 SEQ ID NO: 101.

In some aspects, the construct comprises a nucleic acid sequence with at least 85%, at least 90%, at least 95%, at least 96%, at least 97%, at least 98% at least 99%, or 100% identity to SEQ ID NO: 102. In some aspects, the construct comprises SEQ ID NO: 102. In some aspects, the construct comprises a nucleic acid sequence with at least 85%, at least 90%, at least 95%, at least 96%, at least 97%, at least 98% at least 99%, or 100% identity to nucleotides 12-4993 of SEQ ID NO: 102. In some aspects, the construct comprises nucleotides 12-4993 of SEQ ID NO: 102.

In some aspects, the construct comprises a nucleic acid sequence with at least 85%, at least 90%, at least 95%, at least 96%, at least 97%, at least 98% at least 99%, or 100% identity to SEQ ID NO: 103. In some aspects, the construct comprises SEQ ID NO: 103. In some aspects, the construct comprises a nucleic acid sequence with at least 85%, at least 90%, at least 95%, at least 96%, at least 97%, at least 98% at least 99%, or 100% identity to nucleotides 12-4496 of SEQ ID NO: 103. In some aspects, the construct comprises nucleotides 12-4496 of SEQ ID NO: 103.

In some aspects, the construct comprises a nucleic acid sequence with at least 85%, at least 90%, at least 95%, at least 96%, at least 97%, at least 98% at least 99%, or 100% identity to SEQ ID NO: 104 In some aspects, the construct comprises SEQ ID NO: 104. In some aspects, the construct comprises a nucleic acid sequence with at least 85%, at least 90%, at least 95%, at least 96%, at least 97%, at least 98% at least 99%, or 100% identity to nucleotides 12-4253 of SEQ ID NO: 104 In some aspects, the construct comprises nucleotides 12-4253 of SEQ ID NO: 104.

In some aspects, the construct comprises a nucleic acid sequence with at least 85%, at least 90%, at least 95%, at least 96%, at least 97%, at least 98% at least 99%, or 100% identity to SEQ ID NO: 105. In some aspects, the construct comprises SEQ ID NO: 105. In some aspects, the construct comprises a nucleic acid sequence with at least 85%, at least 90%, at least 95%, at least 96%, at least 97%, at least 98% at least 99%, or 100% identity to nucleotides 12-4320 of SEQ ID NO: 105. In some aspects, the construct comprises nucleotides 12-4320 SEQ ID NO: 105.

In some aspects, the construct comprises a nucleic acid sequence with at least 85%, at least 90%, at least 95%, at least 96%, at least 97%, at least 98% at least 99%, or 100% identity to SEQ ID NO: 106. In some aspects, the construct comprises SEQ ID NO: 106. In some aspects, the construct comprises a nucleic acid sequence with at least 85%, at least 90%, at least 95%, at least 96%, at least 97%, at least 98% at least 99%, or 100% identity to nucleotides 12-4464 of SEQ ID NO: 106. In some aspects, the construct comprises nucleotides 12-4464 of SEQ ID NO: 106.

In some aspects, the construct comprises a nucleic acid sequence with at least 85%, at least 90%, at least 95%, at least 96%, at least 97%, at least 98% at least 99%, or 100% identity to SEQ ID NO: 107 In some aspects, the construct comprises SEQ ID NO: 107. In some aspects, the construct comprises a nucleic acid sequence with at least 85%, at least 90%, at least 95%, at least 96%, at least 97%, at least 98% at least 99%, or 100% identity to nucleotides 12-4328 of SEQ ID NO: 107 In some aspects, the construct comprises nucleotides 12-4328 of SEQ ID NO: 107.

One skilled in the art would recognize that constructs may undergo additional modifications including codon-optimization, introduction of novel but functionally equivalent (e.g., silent mutations), addition of reporter sequences, and/or other routine modification.

In some aspects, an exemplary rAAVAnc80 particle comprises a construct represented by SEQ ID NO: 82.

In one aspect, an exemplary construct comprises: a 5′ ITR exemplified by SEQ ID NO: 52, optionally a cloning site exemplified by SEQ ID NO: 70, a CAG enhancer/promoter exemplified by SEQ ID NO: 14, optionally a cloning site exemplified by SEQ ID NO: 72, a GJB2 5′UTR sequence exemplified by SEQ ID NO: 66, optionally a cloning site exemplified by SEQ ID NO: 73, a GJB2 coding region, a linker sequence exemplified by SEQ ID NO: 77, a FLAG sequence with stop codon exemplified by SEQ ID NO: 81, a 3′ UTR exemplified by SEQ ID NO: 67, optionally a cloning site exemplified by SEQ ID NO: 75, a poly(A) site exemplified by SEQ ID NO: 25, optionally a cloning site exemplified by SEQ ID NO: 76, and a 3′ ITR exemplified by SEQ ID NO: 53.

In some aspects, an exemplary rAAVAnc80 particle comprises a construct represented by SEQ ID NO: 83.

In one aspect, an exemplary construct comprises: a 5′ ITR exemplified by SEQ ID NO: 52, optionally a cloning site exemplified by SEQ ID NO: 70, a CMV/CBA enhancer/promoter exemplified by SEQ ID NO: 12, a chimeric intron exemplified by SEQ ID NO: 64, optionally a cloning site exemplified by SEQ ID NO: 72, a GJB2 5′UTR sequence exemplified by SEQ ID NO: 66, optionally a cloning site exemplified by SEQ ID NO: 73, a GJB2 coding region, a linker sequence exemplified by SEQ ID NO: 77, optionally a FLAG sequence with stop codon exemplified by SEQ ID NO: 81, a 3′ UTR exemplified by SEQ ID NO: 67, optionally a cloning site exemplified by SEQ ID NO: 75, a poly(A) site exemplified by SEQ ID NO: 25, optionally a cloning site exemplified by SEQ ID NO: 76, and a 3′ ITR exemplified by SEQ ID NO: 53.

In some aspects, an exemplary rAAVAnc80 particle comprises a construct represented by SEQ ID NO: 84.

In one aspect, an exemplary construct comprises: a 5′ ITR exemplified by SEQ ID NO: 52, optionally a cloning site exemplified by SEQ ID NO: 70, a CMV enhancer exemplified by SEQ ID NO: 63, a human GJB2 promoter exemplified by SEQ ID NO: 61, optionally a cloning site exemplified by SEQ ID NO: 72, a GJB2 5′UTR sequence exemplified by SEQ ID NO: 66, optionally a cloning site exemplified by SEQ ID NO: 73, a GJB2 coding region, a linker sequence exemplified by SEQ ID NO: 77, optionally a FLAG sequence with stop codon exemplified by SEQ ID NO: 81, a 3′ UTR exemplified by SEQ ID NO: 67, optionally a cloning site exemplified by SEQ ID NO: 75, a poly(A) site exemplified by SEQ ID NO: 25, optionally a cloning site exemplified by SEQ ID NO: 76, and a 3′ ITR exemplified by SEQ ID NO: 53.

In some aspects, an exemplary rAAVAnc80 particle comprises a construct represented by SEQ ID NO: 87.

In one aspect, an exemplary construct comprises: a 5′ ITR exemplified by SEQ ID NO: 52, optionally a cloning site exemplified by SEQ ID NO: 70, a human GFAP enhancer-promoter exemplified by SEQ ID NO: 62, optionally a cloning site exemplified by SEQ ID NO: 72, a GJB2 5′UTR sequence exemplified by SEQ ID NO: 66, optionally a cloning site exemplified by SEQ ID NO: 73, a GJB2 coding region, a linker sequence exemplified by SEQ ID NO: 77, optionally a FLAG sequence with stop codon exemplified by SEQ ID NO: 81, a destabilization domain exemplified by SEQ ID NO: 80, a 3′ UTR exemplified by SEQ ID NO: 68, optionally a cloning site exemplified by SEQ ID NO: 34, a poly(A) site exemplified by SEQ ID NO: 25, optionally a cloning site exemplified by SEQ ID NO: 76, and a 3′ ITR exemplified by SEQ ID NO: 53.

In some aspects, the rAAVAnc80 particle comprises a construct comprising the nucleic acid sequence of SEQ ID NO: 61.

In one aspect, the construct comprises a 5′ ITR comprising the nucleic acid sequence of SEQ ID NO: 52, optionally a cloning site comprising the nucleic acid sequence of SEQ ID NO: 71, a GDF6 promoter sequence comprising the nucleic acid sequence of SEQ ID NO: 90; a hGJB2 minimal promoter comprising the nucleic acid sequence of SEQ ID NO: 86, optionally a cloning site comprising the nucleic acid sequence of SEQ ID NO: 85; optionally a synthetic barcode comprising the nucleic acid sequence of SEQ ID NO: 62; a 5′UTR sequence comprising the nucleic acid sequence of SEQ ID NO: 66, a GJB2 coding region, a linker sequence exemplified by SEQ ID NO: 77, optionally a FLAG sequence with stop codon comprising the nucleic acid sequence of SEQ ID NO: 81, a 3′ UTR comprising the nucleic acid sequence of SEQ ID NO: 67, a poly(A) comprising the nucleic acid sequence of SEQ ID NO: 25, optionally a cloning site comprising the nucleic acid sequence of SEQ ID NO: 76, and a 3′ ITR comprising the nucleic acid sequence of SEQ ID NO: 53.

In some aspects, the rAAVAnc80 particle comprises a construct comprising the nucleic acid sequence of SEQ ID NO: 54.

In one aspect, the construct comprises a 5′ ITR comprising the nucleic acid sequence of SEQ ID NO: 52, optionally a cloning site comprising the nucleic acid sequence of SEQ ID NO: 71, a IGFBP2 promoter sequence comprising the nucleic acid sequence of SEQ ID NO: 57; a hGJB2 minimal promoter comprising the nucleic acid sequence of SEQ ID NO: 86, optionally a cloning site comprising the nucleic acid sequence of SEQ ID NO: 85; optionally a synthetic barcode comprising the nucleic acid sequence of SEQ ID NO: 55; a 5′UTR sequence comprising the nucleic acid sequence of SEQ ID NO: 66, a GJB2 coding region comp, a linker sequence exemplified by SEQ ID NO: 77, optionally a FLAG sequence with stop codon comprising the nucleic acid sequence of SEQ ID NO: 81, a 3′ UTR comprising the nucleic acid sequence of SEQ ID NO: 67, a poly(A) comprising the nucleic acid sequence of SEQ ID NO: 25, optionally a cloning site comprising the nucleic acid sequence of SEQ ID NO: 76, and a 3′ ITR comprising the nucleic acid sequence of SEQ ID NO: 53.

In some aspects, the rAAVAnc80 particle comprises a construct comprising the nucleic acid sequence of SEQ ID NO: 17.

In one aspect, the construct comprises a 5′ ITR comprising the nucleic acid sequence of SEQ ID NO: 52, optionally a cloning site comprising the nucleic acid sequence of SEQ ID NO: 71, a RBP7 promoter sequence comprising the nucleic acid sequence of SEQ ID NO: 28; a hGJB2 minimal promoter comprising the nucleic acid sequence of SEQ ID NO: 86, optionally a cloning site comprising the nucleic acid sequence of SEQ ID NO: 85; optionally a synthetic barcode comprising the nucleic acid sequence of SEQ ID NO: 27; a 5′UTR sequence comprising the nucleic acid sequence of SEQ ID NO: 66, a GJB2 coding region, a linker sequence exemplified by SEQ ID NO: 77, optionally a FLAG sequence with stop codon comprising the nucleic acid sequence of SEQ ID NO: 81, a 3′ UTR comprising the nucleic acid sequence of SEQ ID NO: 67, a poly(A) comprising the nucleic acid sequence of SEQ ID NO: 25, optionally a cloning site comprising the nucleic acid sequence of SEQ ID NO: 76, and a 3′ ITR comprising the nucleic acid sequence of SEQ ID NO: 53.

In some aspects, the rAAVAnc80 particle comprises a construct comprising the nucleic acid sequence of SEQ ID NO: 17.

In one aspect, the construct comprises a 5′ ITR comprising the nucleic acid sequence of SEQ ID NO: 52, optionally a cloning site comprising the nucleic acid sequence of SEQ ID NO: 71, a GJB6 promoter sequence comprising the nucleic acid sequence of SEQ ID NO: 16; a hGJB2 minimal promoter comprising the nucleic acid sequence of SEQ ID NO: 86, optionally a cloning site comprising the nucleic acid sequence of SEQ ID NO: 85; optionally a synthetic barcode comprising the nucleic acid sequence of SEQ ID NO: 41; a 5′UTR sequence comprising the nucleic acid sequence of SEQ ID NO: 66, a GJB2 coding region, a linker sequence exemplified by SEQ ID NO: 77, optionally a FLAG sequence with stop codon comprising the nucleic acid sequence of SEQ ID NO: 81, a 3′ UTR comprising the nucleic acid sequence of SEQ ID NO: 67, a poly(A) comprising the nucleic acid sequence of SEQ ID NO: 25, optionally a cloning site comprising the nucleic acid sequence of SEQ ID NO: 76, and a 3′ ITR comprising the nucleic acid sequence of SEQ ID NO: 53.

In some aspects, the rAAVAnc80 particle comprises a construct comprising the nucleic acid sequence of SEQ ID NO: 7.

In one aspect, the construct comprises a 5′ ITR comprising the nucleic acid sequence of SEQ ID NO: 52, optionally a cloning site comprising the nucleic acid sequence of SEQ ID NO: 71, a PARM1 promoter sequence comprising the nucleic acid sequence of SEQ ID NO: 40; a hGJB2 minimal promoter comprising the nucleic acid sequence of SEQ ID NO: 86, optionally a cloning site comprising the nucleic acid sequence of SEQ ID NO: 85; optionally a synthetic barcode comprising the nucleic acid sequence of SEQ ID NO: 39; a 5′UTR sequence comprising the nucleic acid sequence of SEQ ID NO: 66, a GJB2 coding region, a linker sequence exemplified by SEQ ID NO: 77, optionally a FLAG sequence with stop codon comprising the nucleic acid sequence of SEQ ID NO: 81, a 3′ UTR comprising the nucleic acid sequence of SEQ ID NO: 67, a poly(A) comprising the nucleic acid sequence of SEQ ID NO: 25, optionally a cloning site comprising the nucleic acid sequence of SEQ ID NO: 76, and a 3′ ITR comprising the nucleic acid sequence of SEQ ID NO: 53.

In some aspects, the rAAVAnc80 particle comprises a construct comprising the nucleic acid sequence of SEQ ID NO: 100.

In one aspect, the construct comprises a 5′ ITR comprising the nucleic acid sequence of SEQ ID NO: 52, optionally a cloning site comprising the nucleic acid sequence of SEQ ID NO: 71, a BACE2 promoter sequence comprising the nucleic acid sequence of SEQ ID NO: 92; a hGJB2 minimal promoter comprising the nucleic acid sequence of SEQ ID NO: 86, optionally a cloning site comprising the nucleic acid sequence of SEQ ID NO: 85, optionally a synthetic barcode comprising the nucleic acid sequence of SEQ ID NO: 108; a 5′UTR sequence comprising the nucleic acid sequence of SEQ ID NO: 66, a GJB2 coding region, optionally a FLAG sequence with stop codon comprising the nucleic acid sequence of SEQ ID NO: 81, a 3′ UTR comprising the nucleic acid sequence of SEQ ID NO: 67, a poly(A) comprising the nucleic acid sequence of SEQ ID NO: 25, optionally a cloning site comprising the nucleic acid sequence of SEQ ID NO: 76, and a 3′ ITR comprising the nucleic acid sequence of SEQ ID NO: 53.

In some aspects, the rAAVAnc80 particle comprises a construct comprising the nucleic acid sequence of SEQ ID NO: 101.

In one aspect, the construct comprises a 5′ ITR comprising the nucleic acid sequence of SEQ ID NO: 52, optionally a cloning site comprising the nucleic acid sequence of SEQ ID NO: 71, a DBI2 promoter sequence comprising the nucleic acid sequence of SEQ ID NO: 93; a hGJB2 minimal promoter comprising the nucleic acid sequence of SEQ ID NO: 86, optionally a cloning site comprising the nucleic acid sequence of SEQ ID NO: 85, optionally a synthetic barcode comprising the nucleic acid sequence of SEQ ID NO: 109; a 5′UTR sequence comprising the nucleic acid sequence of SEQ ID NO: 66, a GJB2 coding region, optionally a FLAG sequence with stop codon comprising the nucleic acid sequence of SEQ ID NO: 81, a 3′ UTR comprising the nucleic acid sequence of SEQ ID NO: 67, a poly(A) comprising the nucleic acid sequence of SEQ ID NO: 25, optionally a cloning site comprising the nucleic acid sequence of SEQ ID NO: 76, and a 3′ ITR comprising the nucleic acid sequence of SEQ ID NO: 53.

In some aspects, the rAAVAnc80 particle comprises a construct comprising the nucleic acid sequence of SEQ ID NO: 102.

In one aspect, the construct comprises a 5′ ITR comprising the nucleic acid sequence of SEQ ID NO: 52, optionally a cloning site comprising the nucleic acid sequence of SEQ ID NO: 71, a FABP3 promoter sequence comprising the nucleic acid sequence of SEQ ID NO: 94; a hGJB2 minimal promoter comprising the nucleic acid sequence of SEQ ID NO: 86, optionally a cloning site comprising the nucleic acid sequence of SEQ ID NO: 85, optionally a synthetic barcode comprising the nucleic acid sequence of SEQ ID NO: 110; a 5′UTR sequence comprising the nucleic acid sequence of SEQ ID NO: 66, a GJB2 coding region, optionally a FLAG sequence with stop codon comprising the nucleic acid sequence of SEQ ID NO: 81, a 3′ UTR comprising the nucleic acid sequence of SEQ ID NO: 67, a poly(A) comprising the nucleic acid sequence of SEQ ID NO: 25, optionally a cloning site comprising the nucleic acid sequence of SEQ ID NO: 76, and a 3′ ITR comprising the nucleic acid sequence of SEQ ID NO: 53.

In some aspects, the rAAVAnc80 particle comprises a construct comprising the nucleic acid sequence of SEQ ID NO: 103.

In one aspect, the construct comprises a 5′ ITR comprising the nucleic acid sequence of SEQ ID NO: 52, optionally a cloning site comprising the nucleic acid sequence of SEQ ID NO: 71, a KLHL14 promoter sequence comprising the nucleic acid sequence of SEQ ID NO: 95; a hGJB2 minimal promoter comprising the nucleic acid sequence of SEQ ID NO: 86, optionally a cloning site comprising the nucleic acid sequence of SEQ ID NO: 85, optionally a synthetic barcode comprising the nucleic acid sequence of SEQ ID NO: 111; a 5′UTR sequence comprising the nucleic acid sequence of SEQ ID NO: 66, a GJB2 coding region, optionally a FLAG sequence with stop codon comprising the nucleic acid sequence of SEQ ID NO: 81, a 3′ UTR comprising the nucleic acid sequence of SEQ ID NO: 67, a poly(A) comprising the nucleic acid sequence of SEQ ID NO: 25, optionally a cloning site comprising the nucleic acid sequence of SEQ ID NO: 76, and a 3′ ITR comprising the nucleic acid sequence of SEQ ID NO: 53.

In some aspects, the rAAVAnc80 particle comprises a construct comprising the nucleic acid sequence of SEQ ID NO: 104.

In one aspect, the construct comprises a 5′ ITR comprising the nucleic acid sequence of SEQ ID NO: 52, optionally a cloning site comprising the nucleic acid sequence of SEQ ID NO: 71, a MMP15 promoter sequence comprising the nucleic acid sequence of SEQ ID NO: 96; a hGJB2 minimal promoter comprising the nucleic acid sequence of SEQ ID NO: 86, optionally a cloning site comprising the nucleic acid sequence of SEQ ID NO: 85, optionally a synthetic barcode comprising the nucleic acid sequence of SEQ ID NO: 112; a 5′UTR sequence comprising the nucleic acid sequence of SEQ ID NO: 66, a GJB2 coding region, optionally a FLAG sequence with stop codon comprising the nucleic acid sequence of SEQ ID NO: 81, a 3′ UTR comprising the nucleic acid sequence of SEQ ID NO: 67, a poly(A) comprising the nucleic acid sequence of SEQ ID NO: 25, optionally a cloning site comprising the nucleic acid sequence of SEQ ID NO: 76, and a 3′ ITR comprising the nucleic acid sequence of SEQ ID NO: 53.

In some aspects, the rAAVAnc80 particle comprises a construct comprising the nucleic acid sequence of SEQ ID NO: 105.

In one aspect, the construct comprises a 5′ ITR comprising the nucleic acid sequence of SEQ ID NO: 52, optionally a cloning site comprising the nucleic acid sequence of SEQ ID NO: 71, a SPARC promoter sequence comprising the nucleic acid sequence of SEQ ID NO: 97; a hGJB2 minimal promoter comprising the nucleic acid sequence of SEQ ID NO: 86, optionally a cloning site comprising the nucleic acid sequence of SEQ ID NO: 85, optionally a synthetic barcode comprising the nucleic acid sequence of SEQ ID NO: 113; a 5′UTR sequence comprising the nucleic acid sequence of SEQ ID NO: 66, a GJB2 coding region, optionally a FLAG sequence with stop codon comprising the nucleic acid sequence of SEQ ID NO: 81, a 3′ UTR comprising the nucleic acid sequence of SEQ ID NO: 67, a poly(A) comprising the nucleic acid sequence of SEQ ID NO: 25, optionally a cloning site comprising the nucleic acid sequence of SEQ ID NO: 76, and a 3′ ITR comprising the nucleic acid sequence of SEQ ID NO: 53.

In some aspects, the rAAVAnc80 particle comprises a construct comprising the nucleic acid sequence of SEQ ID NO: 106.

In one aspect, the construct comprises a 5′ ITR comprising the nucleic acid sequence of SEQ ID NO: 52, optionally a cloning site comprising the nucleic acid sequence of SEQ ID NO: 71, a TSPAN8 promoter sequence comprising the nucleic acid sequence of SEQ ID NO: 98; a hGJB2 minimal promoter comprising the nucleic acid sequence of SEQ ID NO: 86, optionally a cloning site comprising the nucleic acid sequence of SEQ ID NO: 85, optionally a synthetic barcode comprising the nucleic acid sequence of SEQ ID NO: 124; a 5′UTR sequence comprising the nucleic acid sequence of SEQ ID NO: 66, a GJB2 coding region, optionally a FLAG sequence with stop codon comprising the nucleic acid sequence of SEQ ID NO: 81, a 3′ UTR comprising the nucleic acid sequence of SEQ ID NO: 67, a poly(A) comprising the nucleic acid sequence of SEQ ID NO: 25, optionally a cloning site comprising the nucleic acid sequence of SEQ ID NO: 76, and a 3′ ITR comprising the nucleic acid sequence of SEQ ID NO: 53.

In some aspects, the rAAVAnc80 particle comprises a construct comprising the nucleic acid sequence of SEQ ID NO: 107.

In one aspect, the construct comprises a 5′ ITR comprising the nucleic acid sequence of SEQ ID NO: 52, optionally a cloning site comprising the nucleic acid sequence of SEQ ID NO: 71, a VIM promoter sequence comprising the nucleic acid sequence of SEQ ID NO: 99; a hGJB2 minimal promoter comprising the nucleic acid sequence of SEQ ID NO: 86, optionally a cloning site comprising the nucleic acid sequence of SEQ ID NO: 85, optionally a synthetic barcode comprising the nucleic acid sequence of SEQ ID NO: 115; a 5′UTR sequence comprising the nucleic acid sequence of SEQ ID NO: 66, a GJB2 coding region, optionally a FLAG sequence with stop codon comprising the nucleic acid sequence of SEQ ID NO: 81, a 3′ UTR comprising the nucleic acid sequence of SEQ ID NO: 67, a poly(A) comprising the nucleic acid sequence of SEQ ID NO: 25, optionally a cloning site comprising the nucleic acid sequence of SEQ ID NO: 76, and a 3′ ITR comprising the nucleic acid sequence of SEQ ID NO: 53.

Exemplary Construct sequence
(SEQ ID NO: 82)
CTGCGCGCTCGCTCGCTCACTGAGGCCGCCCGGGCGTCGGGCGAC
CTTTGGTCGCCCGGCCTCAGTGAGCGAGCGAGCGCGCAGAGAGGG
AGTGGCCAACTCCATCACTAGGGGTTCCTGCGGCCGCACGCGTGA
CATTGATTATTGACTAGTTATTAATAGTAATCAATTACGGGGTCA
TTAGTTCATAGCCCATATATGGAGTTCCGCGTTACATAACTTACG
GTAAATGGCCCGCCTGGCTGACCGCCCAACGACCCCCGCCCATTG
ACGTCAATAATGACGTATGTTCCCATAGTAACGCCAATAGGGACT
TTCCATTGACGTCAATGGGTGGACTATTTACGGTAAACTGCCCAC
TTGGCAGTACATCAAGTGTATCATATGCCAAGTACGCCCCCTATT
GACGTCAATGACGGTAAATGGCCCGCCTGGCATTATGCCCAGTAC
ATGACCTTATGGGACTTTCCTACTTGGCAGTACATCTACGTATTA
GTCATCGCTATTACCATGGGTCGAGGTGAGCCCCACGTTCTGCTT
CACTCTCCCCATCTCCCCCCCCTCCCCACCCCCAATTTTGTATTT
ATTTATTTTTTAATTATTTTGTGCAGCGATGGGGGCGGGGGGGGG
GGGGGCGCGCGCCAGGCGGGGCGGGGCGGGGCGAGGGGCGGGGCG
GGGCGAGGCGGAGAGGTGCGGCGGCAGCCAATCAGAGCGGCGCGC
TCCGAAAGTTTCCTTTTATGGCGAGGCGGCGGCGGCGGCGGCCCT
ATAAAAAGCGAAGCGCGCGGCGGGCGGGAGTCGCTGCGTTGCCTT
CGCCCCGTGCCCCGCTCCGCGCCGCCTCGCGCCGCCCGCCCCGGC
TCTGACTGACCGCGTTACTCCCACAGGTGAGCGGGCGGGACGGCC
CTTCTCCTCCGGGCTGTAATTAGCGCTTGGTTTAATGACGGCTCG
TTTCTTTTCTGTGGCTGCGTGAAAGCCTTAAAGGGCTCCGGGAGG
GCCCTTTGTGCGGGGGGGAGCGGCTCGGGGGGTGCGTGCGTGTGT
GTGTGCGTGGGGAGCGCCGCGTGCGGCCCGCGCTGCCCGGCGGCT
GTGAGCGCTGCGGGCGCGGCGCGGGGCTTTGTGCGCTCCGCGTGT
GCGCGAGGGGAGCGCGGCCGGGGGCGGTGCCCCGCGGTGCGGGGG
GGCTGCGAGGGGAACAAAGGCTGCGTGCGGGGTGTGTGCGTGGGG
GGGTGAGCAGGGGGTGTGGGCGCGGCGGTCGGGCTGTAACCCCCC
CCTGCACCCCCCTCCCCGAGTTGCTGAGCACGGCCCGGCTTCGGG
TGCGGGGCTCCGTGCGGGGCGTGGCGCGGGGCTCGCCGTGCCGGG
CGGGGGGTGGCGGCAGGTGGGGGTGCCGGGCGGGGGGGGGCCGCC
TCGGGCCGGGGAGGGCTCGGGGGAGGGGCGCGGCGGCCCCCGGAG
CGCCGGCGGCTGTCGAGGCGCGGCGAGCCGCAGCCATTGCCTTTT
ATGGTAATCGTGCGAGAGGGCGCAGGGACTTCCTTTGTCCCAAAT
CTGTGCGGAGCCGAAATCTGGGAGGCGCCGCCGCACCCCCTCTAG
CGGGCGCGGGGCGAAGCGGTGCGGCGCCGGCAGGAAGGAAATGGG
GGGGGAGGGCCTTCGTGCGTCGCCGCGCCGCCGTCCCCTTCTCCC
TCTCCAGCCTCGGGGCTGTCCGCGGGGGGACGGCTGCCTTCGGGG
GGGACGGGGCAGGGCGGGGTTCGGCTTCTGGCGTGTGACCGGCGG
CTCTAGAGCCTCTGCTAACCATGTTCATGCCTTCTTCTTTTTCCT
ACAGCTCCTGGGCAACGTGCTGGTTATTGTGACCGGTGTTGCGGC
CCCGCAGCGCCCGCGCGCTCCTCTCCCCGACTCGGAGCCCCTCGG
CGGCGCCCGGCCCAGGACCCGCCTAGGAGCGCAGGAGCCCCAGCG
CAGAGACCCCAACGCCGAGACCCCCGCCCCGGCCCCGCCGCGCTT
CCTCCCGACGCAGTTTAGGACCCTTGTTCGCGAAGAGGTGGTGTG
CGGCTGAGACCCGCGTCCTCAGGACGGTTCCATCAGTGCCTCGAT
CCTGCCCCACTGGAGGAGGAAGGCAGCCCGAACAGCGCTCACCTA
ACTAACAGCTGCTGAGAGCTGGGTTCCGTGGCCATGCACCTGGGA
CTGCCTTGAGAAGCGTGAGCAAACCGCCCAGAGTAGAAGCGCTAG
CCACCATGGATTGGGGCACGCTGCAGACGATCCTGGGGGGTGTGA
ACAAACACTCCACCAGCATTGGAAAGATCTGGCTCACCGTCCTCT
TCATTTTTCGCATTATGATCCTCGTTGTGGCTGCAAAGGAGGTGT
GGGGAGATGAGCAGGCCGACTTTGTCTGCAACACCCTGCAGCCAG
GCTGCAAGAACGTGTGCTACGATCACTACTTCCCCATCTCCCACA
TCCGGCTATGGGCCCTGCAGCTGATCTTCGTGTCCACGCCAGCGC
TCCTAGTGGCCATGCACGTGGCCTACCGGAGACATGAGAAGAAGA
GGAAGTTCATCAAGGGGGAGATAAAGAGTGAATTTAAGGACATCG
AGGAGATCAAAACCCAGAAGGTCCGCATCGAAGGCTCCCTGTGGT
GGACCTACACAAGCAGCATCTTCTTCCGGGTCATCTTCGAAGCCG
CCTTCATGTACGTCTTCTATGTCATGTACGACGGCTTCTCCATGC
AGCGGCTGGTGAAGTGCAACGCCTGGCCTTGTCCCAACACTGTGG
ACTGCTTTGTGTCCCGGCCCACGGAGAAGACTGTCTTCACAGTGT
TCATGATTGCAGTGTCTGGAATTTGCATCCTGCTGAATGTCACTG
AATTGTGTTATTTGCTAATTAGATATTGTTCTGGGAAGTCAAAAA
AGCCAGTTGGATCCCGGGCTGACTACAAAGACCATGACGGTGATT
ATAAAGATCATGACATCGACTACAAGGATGACGATGACAAGTAAG
AAATAGACAGCATGAGAGGGATGAGGCAACCCGTGCTCAGCTGTC
AAGGCTCAGTCGCTAGCATTTCCCAACACAAAGATTCTGACCTTA
AATGCAACCATTTGAAACCCCTGTAGGCCTCAGGTGAAACTCCAG
ATGCCACAATGGAGCTCTGCTCCCCTAAAGCCTCAAAACAAAGGC
CTAATTCTATGCCTGTCTTAATTTTCTTTCACTTAAGTTAGTTCC
ACTGAGACCCCAGGCTGTTAGGGGTTATTGGTGTAAGGTACTTTC
ATATTTTAAACAGAGGATATCGGCATTTGTTTCTTTCTCTGAGGA
CAAGAGAAAAAAGCCAGGTTCCACAGAGGACACAGAGAAGGTTTG
GGTGTCCTCCTGGGGTTCTTTTTGCCAACTTTCCCCACGTTAAAG
GTGAACATTGGTTCTTTCATTTGCTTTGGAAGTTTTAATCTCTAA
CAGTGGACAAAGTTACCAGTGCCTTAAACTCTGTTACACTTTTTG
GAAGTGAAAACTTTGTAGTATGATAGGTTATTTTGATGTAAAGAT
GTTCTGGATACCATTATATGTTCCCCCTGTTTCAGAGGCTCAGAT
TGTAATATGTAAATGGTATGTCATTCGCTACTATGATTTAATTTG
AAATATGGTCTTTTGGTTATGAATACTTTGCAGCACAGCTGAGAG
GCTGTCTGTTGTATTCATTGTGGTCATAGCACCTAACAACATTGT
AGCCTCAATCGAGTGAGACAGACTAGAAGTTCCTAGTGATGGCTT
ATGATAGCAAATGGCCTCATGTCAAATATTTAGATGTAATTTTGT
GTAAGAAATACAGACTGGATGTACCACCAACTACTACCTGTAATG
ACAGGCCTGTCCAACACATCTCCCTTTTCCATGACTGTGGTAGCC
AGCATCGGAAAGAACGCTGATTTAAAGAGGTCGCTTGGGAATTTT
ATTGACACAGTACCATTTAATGGGGAGGACAAAATGGGGCAGGGG
AGGGAGAAGTTTCTGTCGTTAAAAACAGATTTGGAAAGACTGGAC
TCTAAAGTCTGTTGATTAAAGATGAGCTTTGTCTACTTCAAAAGT
TTGTTTGCTTACCCCTTCAGCCTCCAATTTTTTAAGTGAAAATAT
AGCTAATAACATGTGAAAAGAATAGAAGCTAAGGTTTAGATAAAT
ATTGAGCAGATCTATAGGAAGATTGAACCTGAATATTGCCATTAT
GCTTGACATGGTTTCCAAAAAATGGTACTCCACATATTTCAGTGA
GGGTAAGTATTTTCCTGTTGTCAAGAATAGCATTGTAAAAGCATT
TTGTAATAATAAAGAATAGCTTTAATGATATGCTTGTAACTAAAA
TAATTTTGTAATGTATCAAATACATTTAAAACATTAAAATATAAT
CTCTATAATAAGAGCTCGCTGATCAGCCTCGACTGTGCCTTCTAG
TTGCCAGCCATCTGTTGTTTGCCCCTCCCCCGTGCCTTCCTTGAC
CCTGGAAGGTGCCACTCCCACTGTCCTTTCCTAATAAAATGAGGA
AATTGCATCGCATTGTCTGAGTAGGTGTCATTCTATTCTGGGGGG
TGGGGTGGGGCAGGACAGCAAGGGGGAGGATTGGGAAGACAATAG
CAGGCATGCTGGGGATGCGGTGGGCTCTATGGAAGCTTGAATTCA
GCTGACGTGCCTCGGACCGCTAGGAACCCCTAGTGATGGAGTTGG
CCACTCCCTCTCTGCGCGCTCGCTCGCTCACTGAGGCCGGGCGAC
CAAAGGTCGCCCGACGCCCGGGCTTTGCCCGGGCGGCCTCAGTGA
GCGAGCGAGCGCGCAG
Exemplary Construct sequence
(SEQ ID NO: 83)
CTGCGCGCTCGCTCGCTCACTGAGGCCGCCCGGGCGTCGGGCGAC
CTTTGGTCGCCCGGCCTCAGTGAGCGAGCGAGCGCGCAGAGAGGG
AGTGGCCAACTCCATCACTAGGGGTTCCTGCGGCCGCACGCGTGA
CATTGATTATTGACTAGTTATTAATAGTAATCAATTACGGGGTCA
TTAGTTCATAGCCCATATATGGAGTTCCGCGTTACATAACTTACG
GTAAATGGCCCGCCTGGCTGACCGCCCAACGACCCCCGCCCATTG
ACGTCAATAATGACGTATGTTCCCATAGTAACGCCAATAGGGACT
TTCCATTGACGTCAATGGGTGGACTATTTACGGTAAACTGCCCAC
TTGGCAGTACATCAAGTGTATCATATGCCAAGTACGCCCCCTATT
GACGTCAATGACGGTAAATGGCCCGCCTGGCATTATGCCCAGTAC
ATGACCTTATGGGACTTTCCTACTTGGCAGTACATCTACGTATTA
GTCATCGCTATTACCATGGGTCGAGGTGAGCCCCACGTTCTGCTT
CACTCTCCCCATCTCCCCCCCCTCCCCACCCCCAATTTTGTATTT
ATTTATTTTTTAATTATTTTGTGCAGCGATGGGGGCGGGGGGGGG
GGGGGCGCGCGCCAGGCGGGGCGGGGCGGGGCGAGGGGCGGGGCG
GGGCGAGGCGGAGAGGTGCGGCGGCAGCCAATCAGAGCGGCGCGC
TCCGAAAGTTTCCTTTTATGGCGAGGCGGCGGCGGCGGCGGCCCT
ATAAAAAGCGAAGCGCGCGGCGGGCGGGAGTCGCTGCGTTGCCTT
CGCCCCGTGCCCCGCTCCGCGCCGCCTCGCGCCGCCCGCCCCGGC
TCTGACTGACCGCGTTACTCCCACAGGTGAGCGGGCGGGACGGCC
CTTCTCCTCCGGGCTGTAATTAGCGCTTGGTTTAATGACGGCTTG
TTTCTTTTCTGTGGCTGCGTGAAAGCCTTGAGGGGCTCCGGGAGC
TAGAGCCTCTGCTAACCATGTTCATGCCTTCTTCTTTTTCCTACA
GCTCCTGGGCAACGTGCTGGTTATTGTGACCGGTGTTGCGGCCCC
GCAGCGCCCGCGCGCTCCTCTCCCCGACTCGGAGCCCCTCGGCGG
CGCCCGGCCCAGGACCCGCCTAGGAGCGCAGGAGCCCCAGCGCAG
AGACCCCAACGCCGAGACCCCCGCCCCGGCCCCGCCGCGCTTCCT
CCCGACGCAGTTTAGGACCCTTGTTCGCGAAGAGGTGGTGTGCGG
CTGAGACCCGCGTCCTCAGGACGGTTCCATCAGTGCCTCGATCCT
GCCCCACTGGAGGAGGAAGGCAGCCCGAACAGCGCTCACCTAACT
AACAGCTGCTGAGAGCTGGGTTCCGTGGCCATGCACCTGGGACTG
CCTTGAGAAGCGTGAGCAAACCGCCCAGAGTAGAAGCGCTAGCCA
CCATGGATTGGGGCACGCTGCAGACGATCCTGGGGGGTGTGAACA
AACACTCCACCAGCATTGGAAAGATCTGGCTCACCGTCCTCTTCA
TTTTTCGCATTATGATCCTCGTTGTGGCTGCAAAGGAGGTGTGGG
GAGATGAGCAGGCCGACTTTGTCTGCAACACCCTGCAGCCAGGCT
GCAAGAACGTGTGCTACGATCACTACTTCCCCATCTCCCACATCC
GGCTATGGGCCCTGCAGCTGATCTTCGTGTCCACGCCAGCGCTCC
TAGTGGCCATGCACGTGGCCTACCGGAGACATGAGAAGAAGAGGA
AGTTCATCAAGGGGGAGATAAAGAGTGAATTTAAGGACATCGAGG
AGATCAAAACCCAGAAGGTCCGCATCGAAGGCTCCCTGTGGTGGA
CCTACACAAGCAGCATCTTCTTCCGGGTCATCTTCGAAGCCGCCT
TCATGTACGTCTTCTATGTCATGTACGACGGCTTCTCCATGCAGC
GGCTGGTGAAGTGCAACGCCTGGCCTTGTCCCAACACTGTGGACT
GCTTTGTGTCCCGGCCCACGGAGAAGACTGTCTTCACAGTGTTCA
TGATTGCAGTGTCTGGAATTTGCATCCTGCTGAATGTCACTGAAT
TGTGTTATTTGCTAATTAGATATTGTTCTGGGAAGTCAAAAAAGC
CAGTTGGATCCCGGGCTGACTACAAAGACCATGACGGTGATTATA
AAGATCATGACATCGACTACAAGGATGACGATGACAAGTAAGAAA
TAGACAGCATGAGAGGGATGAGGCAACCCGTGCTCAGCTGTCAAG
GCTCAGTCGCTAGCATTTCCCAACACAAAGATTCTGACCTTAAAT
GCAACCATTTGAAACCCCTGTAGGCCTCAGGTGAAACTCCAGATG
CCACAATGGAGCTCTGCTCCCCTAAAGCCTCAAAACAAAGGCCTA
ATTCTATGCCTGTCTTAATTTTCTTTCACTTAAGTTAGTTCCACT
GAGACCCCAGGCTGTTAGGGGTTATTGGTGTAAGGTACTTTCATA
TTTTAAACAGAGGATATCGGCATTTGTTTCTTTCTCTGAGGACAA
GAGAAAAAAGCCAGGTTCCACAGAGGACACAGAGAAGGTTTGGGT
GTCCTCCTGGGGTTCTTTTTGCCAACTTTCCCCACGTTAAAGGTG
AACATTGGTTCTTTCATTTGCTTTGGAAGTTTTAATCTCTAACAG
TGGACAAAGTTACCAGTGCCTTAAACTCTGTTACACTTTTTGGAA
GTGAAAACTTTGTAGTATGATAGGTTATTTTGATGTAAAGATGTT
CTGGATACCATTATATGTTCCCCCTGTTTCAGAGGCTCAGATTGT
AATATGTAAATGGTATGTCATTCGCTACTATGATTTAATTTGAAA
TATGGTCTTTTGGTTATGAATACTTTGCAGCACAGCTGAGAGGCT
GTCTGTTGTATTCATTGTGGTCATAGCACCTAACAACATTGTAGC
CTCAATCGAGTGAGACAGACTAGAAGTTCCTAGTGATGGCTTATG
ATAGCAAATGGCCTCATGTCAAATATTTAGATGTAATTTTGTGTA
AGAAATACAGACTGGATGTACCACCAACTACTACCTGTAATGACA
GGCCTGTCCAACACATCTCCCTTTTCCATGACTGTGGTAGCCAGC
ATCGGAAAGAACGCTGATTTAAAGAGGTCGCTTGGGAATTTTATT
GACACAGTACCATTTAATGGGGAGGACAAAATGGGGCAGGGGAGG
GAGAAGTTTCTGTCGTTAAAAACAGATTTGGAAAGACTGGACTCT
AAAGTCTGTTGATTAAAGATGAGCTTTGTCTACTTCAAAAGTTTG
TTTGCTTACCCCTTCAGCCTCCAATTTTTTAAGTGAAAATATAGC
TAATAACATGTGAAAAGAATAGAAGCTAAGGTTTAGATAAATATT
GAGCAGATCTATAGGAAGATTGAACCTGAATATTGCCATTATGCT
TGACATGGTTTCCAAAAAATGGTACTCCACATATTTCAGTGAGGG
TAAGTATTTTCCTGTTGTCAAGAATAGCATTGTAAAAGCATTTTG
TAATAATAAAGAATAGCTTTAATGATATGCTTGTAACTAAAATAA
TTTTGTAATGTATCAAATACATTTAAAACATTAAAATATAATCTC
TATAATAAGAGCTCGCTGATCAGCCTCGACTGTGCCTTCTAGTTG
CCAGCCATCTGTTGTTTGCCCCTCCCCCGTGCCTTCCTTGACCCT
GGAAGGTGCCACTCCCACTGTCCTTTCCTAATAAAATGAGGAAAT
TGCATCGCATTGTCTGAGTAGGTGTCATTCTATTCTGGGGGGTGG
GGTGGGGCAGGACAGCAAGGGGGAGGATTGGGAAGACAATAGCAG
GCATGCTGGGGATGCGGTGGGCTCTATGGAAGCTTGAATTCAGCT
GACGTGCCTCGGACCGCTAGGAACCCCTAGTGATGGAGTTGGCCA
CTCCCTCTCTGCGCGCTCGCTCGCTCACTGAGGCCGGGCGACCAA
AGGTCGCCCGACGCCCGGGCTTTGCCCGGGCGGCCTCAGTGAGCG
AGCGAGCGCGCAG
Exemplary Construct sequence
(SEQ ID NO: 84)
CTGCGCGCTCGCTCGCTCACTGAGGCCGCCCGGGCGTCGGGCGAC
CTTTGGTCGCCCGGCCTCAGTGAGCGAGCGAGCGCGCAGAGAGGG
AGTGGCCAACTCCATCACTAGGGGTTCCTGCGGCCGCACGCGTGA
CATTGATTATTGACTAGTTATTAATAGTAATCAATTACGGGGTCA
TTAGTTCATAGCCCATATATGGAGTTCCGCGTTACATAACTTACG
GTAAATGGCCCGCCTGGCTGACCGCCCAACGACCCCCGCCCATTG
ACGTCAATAATGACGTATGTTCCCATAGTAACGCCAATAGGGACT
TTCCATTGACGTCAATGGGTGGACTATTTACGGTAAACTGCCCAC
TTGGCAGTACATCAAGTGTATCATATGCCAAGTACGCCCCCTATT
GACGTCAATGACGGTAAATGGCCCGCCTGGCATTATGCCCAGTAC
ATGACCTTATGGGACTTTCCTACTTGGCAGTACATCTACGTATTA
GTCATCGCTATTACCATGGGTAAGCTTCCGCAGAATCCTATCAGT
TTCCCCCTTTCGTGCTGTGTGCATCGAGCAGGAAGGGGCTTGGCA
GGTTTTACCTGCCCTCTTTCCTTTCTGAAAAGTCTGGGCCTCCTC
ACCCCGAAAGGAGTCACCTCCTTGCAGTTCCCCAGTTGCGAAAAG
AGGAGGAAGTTGGCTGGGCCGGGGGCCGCGGGGGGCACCCTCCGC
AGATGGCGGGACCCCCCTGCCGGCCATGGCAAAAACGAGGCTTGT
CTCTCCCACCGCCCCCAACCTTAGTCCTTGGCACATTGTTGAAAG
TAATTGAATAAAATCGGAAATTCGAGAAGGCGTTCGTTCGGATTG
GTGAGATTTTGAGGGGAGAAAGAAGCGGGGACTTCGCCGGCACCA
GCGGCGCCCCCTCCTCGGCCACCGTTAACCCCCATTCCAGAGGGC
ACTGCCCCGCCACCCAGCCTAGGTCCCCCTGCGAGAGCCTCGCGG
GCCCGCGCAGCCTCCGCGACTCGAACAGATCTTCAGTCCTTGGAG
GAATGCCTGTTTCTCTAACAATAAAAAATTAAAGAAGCGCTCATA
AATGCCAAGTCCTCTCGCACTATGCGGAGTACAGAGGACAACGAC
CACAGCCATCCCTGAACCCCGCCCACGGCACAGCGCCGGAGCCGG
GGTCTGGGGCGCCGCTTCCTGGGGGGTCCCGACTCTCAGCCGCCC
CCGCTTCACCCGGGCCGCCAAGGGGCTGGGGGAGGCGGCGCTCGG
GGTAACCGGGGGAGACTCAGGGCGCTGGGGGCACTTGGGGAACTC
ATGGGGGCTCAAAGGAACTAGGAGATCGGGACCTCGAAGGGGACT
TGGGGGGTTCGGGGCTTTCGGGGGCGGTCGGGGGTTCGCGGACCC
GGGAAGCTCTGAGGACCCAGAGGCCGGGCGCGCTCCGCCCGCGGC
GCCGCCCCCTCCGTAACTTTCCCAGTCTCCGAGGGAAGAGGCGGG
GTGTGGGGTGCGGTTAAAAGGCGCCACGGCGGGAGACAGGTCTCC
TGGGCAACGTGCTGGTTATTGTGACCGGTGTTGCGGCCCCGCAGC
GCCCGCGCGCTCCTCTCCCCGACTCGGAGCCCCTCGGCGGCGCCC
GGCCCAGGACCCGCCTAGGAGCGCAGGAGCCCCAGCGCAGAGACC
CCAACGCCGAGACCCCCGCCCCGGCCCCGCCGCGCTTCCTCCCGA
CGCAGTTTAGGACCCTTGTTCGCGAAGAGGTGGTGTGCGGCTGAG
ACCCGCGTCCTCAGGACGGTTCCATCAGTGCCTCGATCCTGCCCC
ACTGGAGGAGGAAGGCAGCCCGAACAGCGCTCACCTAACTAACAG
CTGCTGAGAGCTGGGTTCCGTGGCCATGCACCTGGGACTGCCTTG
AGAAGCGTGAGCAAACCGCCCAGAGTAGAAGCGCTAGCCACCATG
GATTGGGGCACGCTGCAGACGATCCTGGGGGGTGTGAACAAACAC
TCCACCAGCATTGGAAAGATCTGGCTCACCGTCCTCTTCATTTTT
CGCATTATGATCCTCGTTGTGGCTGCAAAGGAGGTGTGGGGAGAT
GAGCAGGCCGACTTTGTCTGCAACACCCTGCAGCCAGGCTGCAAG
AACGTGTGCTACGATCACTACTTCCCCATCTCCCACATCCGGCTA
TGGGCCCTGCAGCTGATCTTCGTGTCCACGCCAGCGCTCCTAGTG
GCCATGCACGTGGCCTACCGGAGACATGAGAAGAAGAGGAAGTTC
ATCAAGGGGGAGATAAAGAGTGAATTTAAGGACATCGAGGAGATC
AAAACCCAGAAGGTCCGCATCGAAGGCTCCCTGTGGTGGACCTAC
ACAAGCAGCATCTTCTTCCGGGTCATCTTCGAAGCCGCCTTCATG
TACGTCTTCTATGTCATGTACGACGGCTTCTCCATGCAGCGGCTG
GTGAAGTGCAACGCCTGGCCTTGTCCCAACACTGTGGACTGCTTT
GTGTCCCGGCCCACGGAGAAGACTGTCTTCACAGTGTTCATGATT
GCAGTGTCTGGAATTTGCATCCTGCTGAATGTCACTGAATTGTGT
TATTTGCTAATTAGATATTGTTCTGGGAAGTCAAAAAAGCCAGTT
GGATCCCGGGCTGACTACAAAGACCATGACGGTGATTATAAAGAT
CATGACATCGACTACAAGGATGACGATGACAAGTAAGAAATAGAC
AGCATGAGAGGGATGAGGCAACCCGTGCTCAGCTGTCAAGGCTCA
GTCGCTAGCATTTCCCAACACAAAGATTCTGACCTTAAATGCAAC
CATTTGAAACCCCTGTAGGCCTCAGGTGAAACTCCAGATGCCACA
ATGGAGCTCTGCTCCCCTAAAGCCTCAAAACAAAGGCCTAATTCT
ATGCCTGTCTTAATTTTCTTTCACTTAAGTTAGTTCCACTGAGAC
CCCAGGCTGTTAGGGGTTATTGGTGTAAGGTACTTTCATATTTTA
AACAGAGGATATCGGCATTTGTTTCTTTCTCTGAGGACAAGAGAA
AAAAGCCAGGTTCCACAGAGGACACAGAGAAGGTTTGGGTGTCCT
CCTGGGGTTCTTTTTGCCAACTTTCCCCACGTTAAAGGTGAACAT
TGGTTCTTTCATTTGCTTTGGAAGTTTTAATCTCTAACAGTGGAC
AAAGTTACCAGTGCCTTAAACTCTGTTACACTTTTTGGAAGTGAA
AACTTTGTAGTATGATAGGTTATTTTGATGTAAAGATGTTCTGGA
TACCATTATATGTTCCCCCTGTTTCAGAGGCTCAGATTGTAATAT
GTAAATGGTATGTCATTCGCTACTATGATTTAATTTGAAATATGG
TCTTTTGGTTATGAATACTTTGCAGCACAGCTGAGAGGCTGTCTG
TTGTATTCATTGTGGTCATAGCACCTAACAACATTGTAGCCTCAA
TCGAGTGAGACAGACTAGAAGTTCCTAGTGATGGCTTATGATAGC
AAATGGCCTCATGTCAAATATTTAGATGTAATTTTGTGTAAGAAA
TACAGACTGGATGTACCACCAACTACTACCTGTAATGACAGGCCT
GTCCAACACATCTCCCTTTTCCATGACTGTGGTAGCCAGCATCGG
AAAGAACGCTGATTTAAAGAGGTCGCTTGGGAATTTTATTGACAC
AGTACCATTTAATGGGGAGGACAAAATGGGGCAGGGGAGGGAGAA
GTTTCTGTCGTTAAAAACAGATTTGGAAAGACTGGACTCTAAAGT
CTGTTGATTAAAGATGAGCTTTGTCTACTTCAAAAGTTTGTTTGC
TTACCCCTTCAGCCTCCAATTTTTTAAGTGAAAATATAGCTAATA
ACATGTGAAAAGAATAGAAGCTAAGGTTTAGATAAATATTGAGCA
GATCTATAGGAAGATTGAACCTGAATATTGCCATTATGCTTGACA
TGGTTTCCAAAAAATGGTACTCCACATATTTCAGTGAGGGTAAGT
ATTTTCCTGTTGTCAAGAATAGCATTGTAAAAGCATTTTGTAATA
ATAAAGAATAGCTTTAATGATATGCTTGTAACTAAAATAATTTTG
TAATGTATCAAATACATTTAAAACATTAAAATATAATCTCTATAA
TAAGAGCTCGCTGATCAGCCTCGACTGTGCCTTCTAGTTGCCAGC
CATCTGTTGTTTGCCCCTCCCCCGTGCCTTCCTTGACCCTGGAAG
GTGCCACTCCCACTGTCCTTTCCTAATAAAATGAGGAAATTGCAT
CGCATTGTCTGAGTAGGTGTCATTCTATTCTGGGGGGTGGGGTGG
GGCAGGACAGCAAGGGGGAGGATTGGGAAGACAATAGCAGGCATG
CTGGGGATGCGGTGGGCTCTATGGAAGCTTGAATTCAGCTGACGT
GCCTCGGACCGCTAGGAACCCCTAGTGATGGAGTTGGCCACTCCC
TCTCTGCGCGCTCGCTCGCTCACTGAGGCCGGGCGACCAAAGGTC
GCCCGACGCCCGGGCTTTGCCCGGGCGGCCTCAGTGAGCGAGCGA
GCGCGCAG
Exemplary Construct sequence
(SEQ ID NO: 87)
CTGCGCGCTCGCTCGCTCACTGAGGCCGCCCGGGCGTCGGGCGAC
CTTTGGTCGCCCGGCCTCAGTGAGCGAGCGAGCGCGCAGAGAGGG
AGTGGCCAACTCCATCACTAGGGGTTCCTGCGGCCGCACGCGTGA
CATTGATTATTGACTAGTTATTAATAGTAATCAATTACGGGGTCA
TTAGTTCATAGCCCATATATGGAGTTCCGCGTTACATAACTTACG
GTAAATGGCCCGCCTGGCTGACCGCCCAACGACCCCCGCCCATTG
ACGTCAATAATGACGTATGTTCCCATAGTAACGCCAATAGGGACT
TTCCATTGACGTCAATGGGTGGACTATTTACGGTAAACTGCCCAC
TTGGCAGTACATCAAGTGTATCATATGCCAAGTACGCCCCCTATT
GACGTCAATGACGGTAAATGGCCCGCCTGGCATTATGCCCAGTAC
ATGACCTTATGGGACTTTCCTACTTGGCAGTACATCTACGTATTA
GTCATCGCTATTACCATGGGTCGAGGTGAGCCCCACGTTCTGCTT
CACTCTCCCCATCTCCCCCCCCTCCCCACCCCCAATTTTGTATTT
ATTTATTTTTTAATTATTTTGTGCAGCGATGGGGGCGGGGGGGGG
GGGGGCGCGCGCCAGGCGGGGCGGGGCGGGGCGAGGGGCGGGGCG
GGGCGAGGCGGAGAGGTGCGGCGGCAGCCAATCAGAGCGGCGCGC
TCCGAAAGTTTCCTTTTATGGCGAGGCGGCGGCGGCGGCGGCCCT
ATAAAAAGCGAAGCGCGCGGCGGGCGGGAGTCGCTGCGTTGCCTT
CGCCCCGTGCCCCGCTCCGCGCCGCCTCGCGCCGCCCGCCCCGGC
TCTGACTGACCGCGTTACTCCCACAGGTGAGCGGGCGGGACGGCC
CTTCTCCTCCGGGCTGTAATTAGCGCTTGGTTTAATGACGGCTCG
TTTCTTTTCTGTGGCTGCGTGAAAGCCTTAAAGGGCTCCGGGAGG
GCCCTTTGTGCGGGGGGGAGCGGCTCGGGGGGTGCGTGCGTGTGT
GTGTGCGTGGGGAGCGCCGCGTGCGGCCCGCGCTGCCCGGCGGCT
GTGAGCGCTGCGGGCGCGGCGCGGGGCTTTGTGCGCTCCGCGTGT
GCGCGAGGGGAGCGCGGCCGGGGGCGGTGCCCCGCGGTGCGGGGG
GGCTGCGAGGGGAACAAAGGCTGCGTGCGGGGTGTGTGCGTGGGG
GGGTGAGCAGGGGGTGTGGGCGCGGCGGTCGGGCTGTAACCCCCC
CCTGCACCCCCCTCCCCGAGTTGCTGAGCACGGCCCGGCTTCGGG
TGCGGGGCTCCGTGCGGGGCGTGGCGCGGGGCTCGCCGTGCCGGG
CGGGGGGTGGCGGCAGGTGGGGGTGCCGGGCGGGGGGGGGCCGCC
TCGGGCCGGGGAGGGCTCGGGGGAGGGGCGCGGCGGCCCCCGGAG
CGCCGGCGGCTGTCGAGGCGCGGCGAGCCGCAGCCATTGCCTTTT
ATGGTAATCGTGCGAGAGGGCGCAGGGACTTCCTTTGTCCCAAAT
CTGTGCGGAGCCGAAATCTGGGAGGCGCCGCCGCACCCCCTCTAG
CGGGCGCGGGGCGAAGCGGTGCGGCGCCGGCAGGAAGGAAATGGG
GGGGGAGGGCCTTCGTGCGTCGCCGCGCCGCCGTCCCCTTCTCCC
TCTCCAGCCTCGGGGCTGTCCGCGGGGGGACGGCTGCCTTCGGGG
GGGACGGGGCAGGGGGGGGTTCGGCTTCTGGCGTGTGACCGGCGG
CTCTAGAGCCTCTGCTAACCATGTTCATGCCTTCTTCTTTTTCCT
ACAGCTCCTGGGCAACGTGCTGGTTATTGTGACCGGTGTTGCGGC
CCCGCAGCGCCCGCGCGCTCCTCTCCCCGACTCGGAGCCCCTCGG
CGGCGCCCGGCCCAGGACCCGCCTAGGAGCGCAGGAGCCCCAGCG
CAGAGACCCCAACGCCGAGACCCCCGCCCCGGCCCCGCCGCGCTT
CCTCCCGACGCAGTTTAGGACCCTTGTTCGCGAAGAGGTGGTGTG
CGGCTGAGACCCGCGTCCTCAGGACGGTTCCATCAGTGCCTCGAT
CCTGCCCCACTGGAGGAGGAAGGCAGCCCGAACAGCGCTCACCTA
ACTAACAGCTGCTGAGAGCTGGGTTCCGTGGCCATGCACCTGGGA
CTGCCTTGAGAAGCGTGAGCAAACCGCCCAGAGTAGAAGCGCTAG
CCACCATGGATTGGGGCACGCTGCAGACGATCCTGGGGGGTGTGA
ACAAACACTCCACCAGCATTGGAAAGATCTGGCTCACCGTCCTCT
TCATTTTTCGCATTATGATCCTCGTTGTGGCTGCAAAGGAGGTGT
GGGGAGATGAGCAGGCCGACTTTGTCTGCAACACCCTGCAGCCAG
GCTGCAAGAACGTGTGCTACGATCACTACTTCCCCATCTCCCACA
TCCGGCTATGGGCCCTGCAGCTGATCTTCGTGTCCACGCCAGCGC
TCCTAGTGGCCATGCACGTGGCCTACCGGAGACATGAGAAGAAGA
GGAAGTTCATCAAGGGGGAGATAAAGAGTGAATTTAAGGACATCG
AGGAGATCAAAACCCAGAAGGTCCGCATCGAAGGCTCCCTGTGGT
GGACCTACACAAGCAGCATCTTCTTCCGGGTCATCTTCGAAGCCG
CCTTCATGTACGTCTTCTATGTCATGTACGACGGCTTCTCCATGC
AGCGGCTGGTGAAGTGCAACGCCTGGCCTTGTCCCAACACTGTGG
ACTGCTTTGTGTCCCGGCCCACGGAGAAGACTGTCTTCACAGTGT
TCATGATTGCAGTGTCTGGAATTTGCATCCTGCTGAATGTCACTG
AATTGTGTTATTTGCTAATTAGATATTGTTCTGGGAAGTCAAAAA
AGCCAGTTGGATCCCGGGCTGACTACAAAGACCATGACGGTGATT
ATAAAGATCATGACATCGACTACAAGGATGACGATGACAAGTAAG
AGCTCAGTGTGAGTTCTACCATTGCCAAACTCGAGCAGTGAATTC
TACCAGTGCCATAGGATCCAGTGTGAGTTCTACCATTGCCAAAGG
TACCCAGTGAATTCTACCAGTGCCATAGTTAACCGCATTGCCCAG
TTGTTAGATTAAGAAATAGACAGCATGAGAGGGATGAGGCAACCC
GTGCTCAGCTGTCAAGGCTCAGTCGCTAGCATTTCCCAACACAAA
GATTCTGACCTTAAATGCAACCATTTGAAACCCCTGTAGGCCTCA
GGTGAAACTCCAGATGCCACAATGGAGCCTCTGCTCCCCTAAAGC
CTCAAAACAAAGGCCTAATTCTATGCCTGTCTTAATTTTCTTTCA
CTTAAGTTAGTTCCACTGAGACCCCAGGCTGTTAGGGGTTATTGG
TGTAAGGTACTTTCATATTTTAAACAGAGGATATCGGCATTTGTT
TCTTTCTCTGAGGACAAGAGAAAAAAGCCAGGTTCCACAGAGGAC
ACAGAGAAGGTTTGGGTGTCCTCCTGGGGTTCTTTTTGCCAACTT
TCCCCACGTTAAAGGTGAACATTGGTTCTTTCATTTGCTTTGGAA
GTTTTAATCTCTAACAGTGGACAAAGTTACCAGTGCCTTAAACTC
TGTTACACTTTTTGGAAGTGAAAACTTTGTAGTATGATAGGTTAT
TTTGATGTAAAGATGTTCTGGATACCATTATATGTTCCCCCTGTT
TCAGAGGCTCAGATTGTAATATGTAAATGGTATGTCATTCGCTAC
TATGATTTAATTTGAAATATGGTCTTTTGGTTATGAATACTTTGC
AGCACAGCTGAGAGGCTGTCTGTTGTATTCATTGTGGTCATAGCA
CCTAACAACATTGTAGCCTCAATCGAGTGAGACAGACTAGAAGTT
CCTAGTGATGGCTTATGATAGCAAATGGCCTCATGTCAAATATTT
AGATGTAATTTTGTGTAAGAAATACAGACTGGATGTACCACCAAC
TACTACCTGTAATGACAGGCCTGTCCAACACATCTCCCTTTTCCA
TGACTGTGGTAGCCAGCATCGGAAAGAACGCTGATTTAAAGAGGT
CGCTTGGGAATTTTATTGACACAGTACCATTTAATGGGGAGGACA
AAATGGGGCAGGGGAGGGAGAAGTTTCTGTCGTTAAAAACAGATT
TGGAAAGACTGGACTCTAAAGTCTGTTGATTAAAGATGAGCTTTG
TCTACTTCAAAAGTTTGTTTGCTTACCCCTTCAGCCTCCAATTTT
TTAAGTGAAAATATAGCTAATAACATGTGAAAAGAATAGAAGCTA
AGGTTTAGATAAATATTGAGCAGATCTATAGGAAGATTGAACCTG
AATATTGCCATTATGCTTGACGCTGATCAGCCTCGACTGTGCCTT
CTAGTTGCCAGCCATCTGTTGTTTGCCCCTCCCCCGTGCCTTCCT
TGACCCTGGAAGGTGCCACTCCCACTGTCCTTTCCTAATAAAATG
AGGAAATTGCATCGCATTGTCTGAGTAGGTGTCATTCTATTCTGG
GGGGTGGGGTGGGGCAGGACAGCAAGGGGGAGGATTGGGAAGACA
ATAGCAGGCATGCTGGGGATGCGGTGGGCTCTATGGAAGCTTGAA
TTCAGCTGACGTGCCTCGGACCGCTAGGAACCCCTAGTGATGGAG
TTGGCCACTCCCTCTCTGCGCGCTCGCTCGCTCACTGAGGCCGGG
CGACCAAAGGTCGCCCGACGCCCGGGCTTTGCCCGGGCGGCCTCA
GTGAGCGAGCGAGCGCGCAG
Exemplary Construct sequence
(SEQ ID NO: 61)
CCTGCAGGCAGCTGCGCGCTCGCTCGCTCACTGAGGCCGCCCGGG
CCTGCAGGCAGCTGCGCGCTCGCTCGCTCACTGAGGCCGCCCGGG
CGTCGGGCGACCTTTGGTCGCCCGGCCTCAGTGAGCGAGCGAGCG
CGTCGGGCGACCTTTGGTCGCCCGGCCTCAGTGAGCGAGCGAGCG
CGCAGAGAGGGAGTGGCCAACTCCATCACTAGGGGTTCCTGCGGC
CGCAGAGAGGGAGTGGCCAACTCCATCACTAGGGGTTCCTGCGGC
CGCACGCGTGGTCCACAGGTAACTCCGTCGGCGTCCACAGGGGGG
CGCACGCGTGGTCCACAGGTAACTCCGTCGGCGTCCACAGGGGGG
CAGGAGATACCATACTGCACAGTTGTACGTCTTCCATCTGTTTGG
CAGGAGATACCATACTGCACAGTTGTACGTCTTCCATCTGTTTGG
TGTAGAAAAATCTAACCACTACAAGAATGCCACGGGCACTGTGGC
TGTAGAAAAATCTAACCACTACAAGAATGCCACGGGCACTGTGGC
AGACAGAAGCAGCGCTACGCCGCATCGCCTTTCAGCGTGCAGGCC
AGACAGAAGCAGCGCTACGCCGCATCGCCTTTCAGCGTGCAGGCC
CAGGAATGAGCGAGGCAGTGGGCGGGGAAGACAGGCACGGGGAAT
CAGGAATGAGCGAGGCAGTGGGCGGGGAAGACAGGCACGGGGAAT
CTGGGGACAGATAAAGGAAACTCGTGATGGGGCGAGGCTGGGCTG
CTGGGGACAGATAAAGGAAACTCGTGATGGGGCGAGGCTGGGCTG
AAGAGAAACAGATTGGGGTAGAGCTGCAAAGGGAGGGGTCCACTG
AAGAGAAACAGATTGGGGTAGAGCTGCAAAGGGAGGGGTCCACTG
GAAGGCGAGGGGGGAGGCCGGGAAGAGAGAGGGTGGGAAGGCAGT
GAAGGCGAGGGGGGAGGCCGGGAAGAGAGAGGGTGGGAAGGCAGT
GTGAGATGGGAGGGCAGTGTGAGAAGAAAAGCAGGCTGGGGAAGA
GTGAGATGGGAGGGCAGTGTGAGAAGAAAAGCAGGCTGGGGAAGA
GGGATTGGAATGCAGAAGGAACTTGGGGAAGGAGGAAGTCCTGCA
GGGATTGGAATGCAGAAGGAACTTGGGGAAGGAGGAAGTCCTGCA
GGCGGGAGGGAAAGAAGAGAGGGGGAGCAGCTAAAGTCTGCGTCA
GGCGGGAGGGAAAGAAGAGAGGGGGAGCAGCTAAAGTCTGCGTCA
GAAGAGGTTGGGGACTGCGAGAGGAGAGGCTGGGGCCTGCAGGGG
GAAGAGGTTGGGGACTGCGAGAGGAGAGGCTGGGGCCTGCAGGGG
AGCGCAGCAGCTTTTAGCATCGATCCAAACTCTAAAGACTCGTGG
AGCGCAGCAGCTTTTAGCATCGATCCAAACTCTAAAGACTCGTGG
CCTTTGCCTGACCTCGAGGGTCGGGAATAGACGCCTGTCTTTGTG
CCTTTGCCTGACCTCGAGGGTCGGGAATAGACGCCTGTCTTTGTG
GAGAGCGATACCCAACCGAGAAAATGGGGCTGTTCCGAGCTGGGC
GAGAGCGATACCCAACCGAGAAAATGGGGCTGTTCCGAGCTGGGC
CCTGCGCCTGGCCCAGGGCGAGGCTTCTCTGGCTCCGGGCTGGCC
CCTGCGCCTGGCCCAGGGCGAGGCTTCTCTGGCTCCGGGCTGGCC
CCTGAGGGGCAGCACGCAGCCTGCAGCAGAGGCGCCTGCTCCAAG
CCTGAGGGGCAGCACGCAGCCTGCAGCAGAGGCGCCTGCTCCAAG
CTGTCTCTTGGGGGCGCCGCCGCCGCTTCCCTCCTCCGGGGCCGC
CTGTCTCTTGGGGGCGCCGCCGCCGCTTCCCTCCTCCGGGGCCGC
TCGCTCCCAGGAAAGTGGAGGCGGCTGGCGAGGACCGAGAGCCGG
TCGCTCCCAGGAAAGTGGAGGCGGCTGGCGAGGACCGAGAGCCGG
GGCCGCGCTGCGGAGGGACCACACCTCCGGGAGTTCGAGGGGGAC
GGCCGCGCTGCGGAGGGACCACACCTCCGGGAGTTCGAGGGGGAC
CCTGGCGCGGCGGGCCAGCCTTTCGGGCCGGCAGCGCCCGCCTTC
CCTGGCGCGGCGGGCCAGCCTTTCGGGCCGGCAGCGCCCGCCTTC
CCCCGGTCAGCGCTTGCGGCCCGCGCCGCGCGCACCGCCCGGCAA
CCCCGGTCAGCGCTTGCGGCCCGCGCCGCGCGCACCGCCCGGCAA
CCCCGCGCGCGTCCCGCGGGGGCGCTGCGTCTTCCTGCCACACCG
CCCCGCGCGCGTCCCGCGGGGGCGCTGCGTCTTCCTGCCACACCG
GCGCACCGCGGCCCCTCTCCCCCACACCTCCGGCCCGCACCACCC
GCGCACCGCGGCCCCTCTCCCCCACACCTCCGGCCCGCACCACCC
GGCTCTCCTCCCACCCTCCCCACCCCTCCTCTGCCCTCCCTCCCC
GGCTCTCCTCCCACCCTCCCCACCCCTCCTCTGCCCTCCCTCCCC
ATTCCTCCCCTCCCGGCGAGGGGCGGGAGGGGGCGTGGCGGGGCC
ATTCCTCCCCTCCCGGCGAGGGGCGGGAGGGGGCGTGGCGGGGCC
GGGGTTTGTGTGGCTGGGACCCGGCTCCTCAAGCTCTGAGGACCC
GGGGTTTGTGTGGCTGGGACCCGGCTCCTCAAGCTCTGAGGACCC
AGAGGCCGGGCGCGCTCCGCCCGCGGCGCCGCCCCCTCCGTAACT
AGAGGCCGGGCGCGCTCCGCCCGCGGCGCCGCCCCCTCCGTAACT
TTCCCAGTCTCCGAGGGAAGAGGCGGGGTGTGGGGTGCGGTTAAA
TTCCCAGTCTCCGAGGGAAGAGGCGGGGTGTGGGGTGCGGTTAAA
AGGCGCCACGGCGGGAGACAGGTCTCACCGGTGTGTCACCGTTGC
AGGCGCCACGGCGGGAGACAGGTCTCACCGGTGTGTCACCGTTGC
GGCCCCGCAGCGCCCGCGCGCTCCTCTCCCCGACTCGGAGCCCCT
GGCCCCGCAGCGCCCGCGCGCTCCTCTCCCCGACTCGGAGCCCCT
CGGCGGCGCCCGGCCCAGGACCCGCCTAGGAGCGCAGGAGCCCCA
CGGCGGCGCCCGGCCCAGGACCCGCCTAGGAGCGCAGGAGCCCCA
GCGCAGAGACCCCAACGCCGAGACCCCCGCCCCGGCCCCGCCGCG
GCGCAGAGACCCCAACGCCGAGACCCCCGCCCCGGCCCCGCCGCG
CTTCCTCCCGACGCAGTTTAGGACCCTTGTTCGCGAAGAGGTGGT
CTTCCTCCCGACGCAGTTTAGGACCCTTGTTCGCGAAGAGGTGGT
GTGCGGCTGAGACCCGCGTCCTCAGGACGGTTCCATCAGTGCCTC
GTGCGGCTGAGACCCGCGTCCTCAGGACGGTTCCATCAGTGCCTC
GATCCTGCCCCACTGGAGGAGGAAGGCAGCCCGAACAGCGCTCAC
GATCCTGCCCCACTGGAGGAGGAAGGCAGCCCGAACAGCGCTCAC
CTAACTAACAGCTGCTGAGAGCTGGGTTCCGTGGCCATGCACCTG
CTAACTAACAGCTGCTGAGAGCTGGGTTCCGTGGCCATGCACCTG
GGACTGCCTTGAGAAGCGTGAGCAAACCGCCCAGAGTAGAAGCGC
GGACTGCCTTGAGAAGCGTGAGCAAACCGCCCAGAGTAGAAGCGC
TAGCCACCATGGATTGGGGCACGCTGCAGACGATCCTGGGGGGTG
TAGCCACCATGGATTGGGGCACGCTGCAGACGATCCTGGGGGGTG
TGAACAAACACTCCACCAGCATTGGAAAGATCTGGCTCACCGTCC
TGAACAAACACTCCACCAGCATTGGAAAGATCTGGCTCACCGTCC
TCTTCATTTTTCGCATTATGATCCTCGTTGTGGCTGCAAAGGAGG
TCTTCATTTTTCGCATTATGATCCTCGTTGTGGCTGCAAAGGAGG
TGTGGGGAGATGAGCAGGCCGACTTTGTCTGCAACACCCTGCAGC
TGTGGGGAGATGAGCAGGCCGACTTTGTCTGCAACACCCTGCAGC
CAGGCTGCAAGAACGTGTGCTACGATCACTACTTCCCCATCTCCC
CAGGCTGCAAGAACGTGTGCTACGATCACTACTTCCCCATCTCCC
ACATCCGGCTATGGGCCCTGCAGCTGATCTTCGTGTCCACGCCAG
ACATCCGGCTATGGGCCCTGCAGCTGATCTTCGTGTCCACGCCAG
CGCTCCTAGTGGCCATGCACGTGGCCTACCGGAGACATGAGAAGA
CGCTCCTAGTGGCCATGCACGTGGCCTACCGGAGACATGAGAAGA
AGAGGAAGTTCATCAAGGGGGAGATAAAGAGTGAATTTAAGGACA
AGAGGAAGTTCATCAAGGGGGAGATAAAGAGTGAATTTAAGGACA
TCGAGGAGATCAAAACCCAGAAGGTCCGCATCGAAGGCTCCCTGT
TCGAGGAGATCAAAACCCAGAAGGTCCGCATCGAAGGCTCCCTGT
GGTGGACCTACACAAGCAGCATCTTCTTCCGGGTCATCTTCGAAG
GGTGGACCTACACAAGCAGCATCTTCTTCCGGGTCATCTTCGAAG
CCGCCTTCATGTACGTCTTCTATGTCATGTACGACGGCTTCTCCA
CCGCCTTCATGTACGTCTTCTATGTCATGTACGACGGCTTCTCCA
TGCAGCGGCTGGTGAAGTGCAACGCCTGGCCTTGTCCCAACACTG
TGCAGCGGCTGGTGAAGTGCAACGCCTGGCCTTGTCCCAACACTG
TGGACTGCTTTGTGTCCCGGCCCACGGAGAAGACTGTCTTCACAG
TGGACTGCTTTGTGTCCCGGCCCACGGAGAAGACTGTCTTCACAG
TGTTCATGATTGCAGTGTCTGGAATTTGCATCCTGCTGAATGTCA
TGTTCATGATTGCAGTGTCTGGAATTTGCATCCTGCTGAATGTCA
CTGAATTGTGTTATTTGCTAATTAGATATTGTTCTGGGAAGTCAA
CTGAATTGTGTTATTTGCTAATTAGATATTGTTCTGGGAAGTCAA
AAAAGCCAGTTGGATCCCGGGCTGACTACAAAGACCATGACGGTG
AAAAGCCAGTTGGATCCCGGGCTGACTACAAAGACCATGACGGTG
ATTATAAAGATCATGACATCGACTACAAGGATGACGATGACAAGT
ATTATAAAGATCATGACATCGACTACAAGGATGACGATGACAAGT
AAGAAATAGACAGCATGAGAGGGATGAGGCAACCCGTGCTCAGCT
AAGAAATAGACAGCATGAGAGGGATGAGGCAACCCGTGCTCAGCT
GTCAAGGCTCAGTCGCTAGCATTTCCCAACACAAAGATTCTGACC
GTCAAGGCTCAGTCGCTAGCATTTCCCAACACAAAGATTCTGACC
TTAAATGCAACCATTTGAAACCCCTGTAGGCCTCAGGTGAAACTC
TTAAATGCAACCATTTGAAACCCCTGTAGGCCTCAGGTGAAACTC
CAGATGCCACAATGGAGCTCTGCTCCCCTAAAGCCTCAAAACAAA
CAGATGCCACAATGGAGCTCTGCTCCCCTAAAGCCTCAAAACAAA
GGCCTAATTCTATGCCTGTCTTAATTTTCTTTCACTTAAGTTAGT
GGCCTAATTCTATGCCTGTCTTAATTTTCTTTCACTTAAGTTAGT
TCCACTGAGACCCCAGGCTGTTAGGGGTTATTGGTGTAAGGTACT
TCCACTGAGACCCCAGGCTGTTAGGGGTTATTGGTGTAAGGTACT
TTCATATTTTAAACAGAGGATATCGGCATTTGTTTCTTTCTCTGA
TTCATATTTTAAACAGAGGATATCGGCATTTGTTTCTTTCTCTGA
GGACAAGAGAAAAAAGCCAGGTTCCACAGAGGACACAGAGAAGGT
GGACAAGAGAAAAAAGCCAGGTTCCACAGAGGACACAGAGAAGGT
TTGGGTGTCCTCCTGGGGTTCTTTTTGCCAACTTTCCCCACGTTA
TTGGGTGTCCTCCTGGGGTTCTTTTTGCCAACTTTCCCCACGTTA
AAGGTGAACATTGGTTCTTTCATTTGCTTTGGAAGTTTTAATCTC
AAGGTGAACATTGGTTCTTTCATTTGCTTTGGAAGTTTTAATCTC
TAACAGTGGACAAAGTTACCAGTGCCTTAAACTCTGTTACACTTT
TAACAGTGGACAAAGTTACCAGTGCCTTAAACTCTGTTACACTTT
TTGGAAGTGAAAACTTTGTAGTATGATAGGTTATTTTGATGTAAA
TTGGAAGTGAAAACTTTGTAGTATGATAGGTTATTTTGATGTAAA
GATGTTCTGGATACCATTATATGTTCCCCCTGTTTCAGAGGCTCA
GATGTTCTGGATACCATTATATGTTCCCCCTGTTTCAGAGGCTCA
GATTGTAATATGTAAATGGTATGTCATTCGCTACTATGATTTAAT
GATTGTAATATGTAAATGGTATGTCATTCGCTACTATGATTTAAT
TTGAAATATGGTCTTTTGGTTATGAATACTTTGCAGCACAGCTGA
TTGAAATATGGTCTTTTGGTTATGAATACTTTGCAGCACAGCTGA
GAGGCTGTCTGTTGTATTCATTGTGGTCATAGCACCTAACAACAT
GAGGCTGTCTGTTGTATTCATTGTGGTCATAGCACCTAACAACAT
TGTAGCCTCAATCGAGTGAGACAGACTAGAAGTTCCTAGTGATGG
TGTAGCCTCAATCGAGTGAGACAGACTAGAAGTTCCTAGTGATGG
CTTATGATAGCAAATGGCCTCATGTCAAATATTTAGATGTAATTT
CTTATGATAGCAAATGGCCTCATGTCAAATATTTAGATGTAATTT
TGTGTAAGAAATACAGACTGGATGTACCACCAACTACTACCTGTA
TGTGTAAGAAATACAGACTGGATGTACCACCAACTACTACCTGTA
ATGACAGGCCTGTCCAACACATCTCCCTTTTCCATGACTGTGGTA
ATGACAGGCCTGTCCAACACATCTCCCTTTTCCATGACTGTGGTA
GCCAGCATCGGAAAGAACGCTGATTTAAAGAGGTCGCTTGGGAAT
GCCAGCATCGGAAAGAACGCTGATTTAAAGAGGTCGCTTGGGAAT
TTTATTGACACAGTACCATTTAATGGGGAGGACAAAATGGGGCAG
TTTATTGACACAGTACCATTTAATGGGGAGGACAAAATGGGGCAG
GGGAGGGAGAAGTTTCTGTCGTTAAAAACAGATTTGGAAAGACTG
GGGAGGGAGAAGTTTCTGTCGTTAAAAACAGATTTGGAAAGACTG
GACTCTAAAGTCTGTTGATTAAAGATGAGCTTTGTCTACTTCAAA
GACTCTAAAGTCTGTTGATTAAAGATGAGCTTTGTCTACTTCAAA
AGTTTGTTTGCTTACCCCTTCAGCCTCCAATTTTTTAAGTGAAAA
AGTTTGTTTGCTTACCCCTTCAGCCTCCAATTTTTTAAGTGAAAA
TATAGCTAATAACATGTGAAAAGAATAGAAGCTAAGGTTTAGATA
TATAGCTAATAACATGTGAAAAGAATAGAAGCTAAGGTTTAGATA
AATATTGAGCAGATCTATAGGAAGATTGAACCTGAATATTGCCAT
AATATTGAGCAGATCTATAGGAAGATTGAACCTGAATATTGCCAT
TATGCTTGACATGGTTTCCAAAAAATGGTACTCCACATATTTCAG
TATGCTTGACATGGTTTCCAAAAAATGGTACTCCACATATTTCAG
TGAGGGTAAGTATTTTCCTGTTGTCAAGAATAGCATTGTAAAAGC
TGAGGGTAAGTATTTTCCTGTTGTCAAGAATAGCATTGTAAAAGC
ATTTTGTAATAATAAAGAATAGCTTTAATGATATGCTTGTAACTA
ATTTTGTAATAATAAAGAATAGCTTTAATGATATGCTTGTAACTA
AAATAATTTTGTAATGTATCAAATACATTTAAAACATTAAAATAT
AAATAATTTTGTAATGTATCAAATACATTTAAAACATTAAAATAT
AATCTCTATAATAAGAGCTCGCTGATCAGCCTCGACTGTGCCTTC
AATCTCTATAATAAGAGCTCGCTGATCAGCCTCGACTGTGCCTTC
TAGTTGCCAGCCATCTGTTGTTTGCCCCTCCCCCGTGCCTTCCTT
TAGTTGCCAGCCATCTGTTGTTTGCCCCTCCCCCGTGCCTTCCTT
GACCCTGGAAGGTGCCACTCCCACTGTCCTTTCCTAATAAAATGA
GACCCTGGAAGGTGCCACTCCCACTGTCCTTTCCTAATAAAATGA
GGAAATTGCATCGCATTGTCTGAGTAGGTGTCATTCTATTCTGGG
GGAAATTGCATCGCATTGTCTGAGTAGGTGTCATTCTATTCTGGG
GGGTGGGGTGGGGCAGGACAGCAAGGGGGAGGATTGGGAAGACAA
GGGTGGGGTGGGGCAGGACAGCAAGGGGGAGGATTGGGAAGACAA
TAGCAGGCATGCTGGGGATGCGGTGGGCTCTATGGAAGCTTGAAT
TAGCAGGCATGCTGGGGATGCGGTGGGCTCTATGGAAGCTTGAAT
TCAGCTGACGTGCCTCGGACCGCTAGGAACCCCTAGTGATGGAGT
TCAGCTGACGTGCCTCGGACCGCTAGGAACCCCTAGTGATGGAGT
TGGCCACTCCCTCTCTGCGCGCTCGCTCGCTCACTGAGGCCGGGC
TGGCCACTCCCTCTCTGCGCGCTCGCTCGCTCACTGAGGCCGGGC
GACCAAAGGTCGCCCGACGCCCGGGCTTTGCCCGGGCGGCCTCAG
GACCAAAGGTCGCCCGACGCCCGGGCTTTGCCCGGGCGGCCTCAG
TGAGCGAGCGAGCGCGCAGCTGCCTGCAGGGGCGCCTGATGCGGT
TGAGCGAGCGAGCGCGCAGCTGCCTGCAGGGGCGCCTGATGCGGT
ATTTTCTCCTTACGCATCTGTGCGGTATTTCACACCGCATACGTC
ATTTTCTCCTTACGCATCTGTGCGGTATTTCACACCGCATACGTC
AAAGCAACCATAGTACGCGCCCTGTAGCGGCGCATTAAGCGCGGC
AAAGCAACCATAGTACGCGCCCTGTAGCGGCGCATTAAGCGCGGC
GGGTGTGGTGGTTACGCGCAGCGTGACCGCTACACTTGCCAGCGC
GGGTGTGGTGGTTACGCGCAGCGTGACCGCTACACTTGCCAGCGC
CCTAGCGCCCGCTCCTTTCGCTTTCTTCCCTTCCTTTCTCGCCAC
CCTAGCGCCCGCTCCTTTCGCTTTCTTCCCTTCCTTTCTCGCCAC
GTTCGCCGGCTTTCCCCGTCAAGCTCTAAATCGGGGGCTCCCTTT
GTTCGCCGGCTTTCCCCGTCAAGCTCTAAATCGGGGGCTCCCTTT
AGGGTTCCGATTTAGTGCTTTACGGCACCTCGACCCCAAAAAACT
AGGGTTCCGATTTAGTGCTTTACGGCACCTCGACCCCAAAAAACT
TGATTTGGGTGATGGTTCACGTAGTGGGCCATCGCCCTGATAGAC
TGATTTGGGTGATGGTTCACGTAGTGGGCCATCGCCCTGATAGAC
GGTTTTTCGCCCTTTGACGTTGGAGTCCACGTTCTTTAATAGTGG
GGTTTTTCGCCCTTTGACGTTGGAGTCCACGTTCTTTAATAGTGG
ACTCTTGTTCCAAACTGGAACAACACTCAACCCTATCTCGGGCTA
ACTCTTGTTCCAAACTGGAACAACACTCAACCCTATCTCGGGCTA
TTCTTTTGATTTATAAGGGATTTTGCCGATTTCGGCCTATTGGTT
TTCTTTTGATTTATAAGGGATTTTGCCGATTTCGGCCTATTGGTT
AAAAAATGAGCTGATTTAACAAAAATTTAACGCGAATTTTAACAA
AAAAAATGAGCTGATTTAACAAAAATTTAACGCGAATTTTAACAA
AATATTAACGTTTACAATTTTATGGTGCACTCTCAGTACAATCTG
AATATTAACGTTTACAATTTTATGGTGCACTCTCAGTACAATCTG
CTCTGATGCCGCATAGTTAAGCCAGCCCCGACACCCGCCAACACC
CTCTGATGCCGCATAGTTAAGCCAGCCCCGACACCCGCCAACACC
CGCTGACGCGCCCTGACGGGCTTGTCTGCTCCCGGCATCCGCTTA
CGCTGACGCGCCCTGACGGGCTTGTCTGCTCCCGGCATCCGCTTA
CAGACAAGCTGTGACCGTCTCCGGGAGCTGCATGTGTCAGAGGTT
CAGACAAGCTGTGACCGTCTCCGGGAGCTGCATGTGTCAGAGGTT
TTCACCGTCATCACCGAAACGCGCGAGACGAAAGGGCCTCGTGAT
TTCACCGTCATCACCGAAACGCGCGAGACGAAAGGGCCTCGTGAT
ACGCCTATTTTTATAGGTTAATGTCATGAACAATAAAACTGTCTG
ACGCCTATTTTTATAGGTTAATGTCATGAACAATAAAACTGTCTG
CTTACATAAACAGTAATACAAGGGGTGTTATGAGCCATATTCAAC
CTTACATAAACAGTAATACAAGGGGTGTTATGAGCCATATTCAAC
GGGAAACGTCGAGGCCGCGATTAAATTCCAACATGGATGCTGATT
GGGAAACGTCGAGGCCGCGATTAAATTCCAACATGGATGCTGATT
TATATGGGTATAAATGGGCTCGCGATAATGTCGGGCAATCAGGTG
TATATGGGTATAAATGGGCTCGCGATAATGTCGGGCAATCAGGTG
CGACAATCTATCGCTTGTATGGGAAGCCCGATGCGCCAGAGTTGT
CGACAATCTATCGCTTGTATGGGAAGCCCGATGCGCCAGAGTTGT
TTCTGAAACATGGCAAAGGTAGCGTTGCCAATGATGTTACAGATG
TTCTGAAACATGGCAAAGGTAGCGTTGCCAATGATGTTACAGATG
AGATGGTCAGACTAAACTGGCTGACGGAATTTATGCCTCTTCCGA
AGATGGTCAGACTAAACTGGCTGACGGAATTTATGCCTCTTCCGA
CCATCAAGCATTTTATCCGTACTCCTGATGATGCATGGTTACTCA
CCATCAAGCATTTTATCCGTACTCCTGATGATGCATGGTTACTCA
CCACTGCGATCCCCGGAAAAACAGCATTCCAGGTATTAGAAGAAT
CCACTGCGATCCCCGGAAAAACAGCATTCCAGGTATTAGAAGAAT
ATCCTGATTCAGGTGAAAATATTGTTGATGCGCTGGCAGTGTTCC
ATCCTGATTCAGGTGAAAATATTGTTGATGCGCTGGCAGTGTTCC
TGCGCCGGTTGCATTCGATTCCTGTTTGTAATTGTCCTTTTAACA
TGCGCCGGTTGCATTCGATTCCTGTTTGTAATTGTCCTTTTAACA
GCGATCGCGTATTTCGTCTCGCTCAGGCGCAATCACGAATGAATA
GCGATCGCGTATTTCGTCTCGCTCAGGCGCAATCACGAATGAATA
ACGGTTTGGTTGATGCGAGTGATTTTGATGACGAGCGTAATGGCT
ACGGTTTGGTTGATGCGAGTGATTTTGATGACGAGCGTAATGGCT
GGCCTGTTGAACAAGTCTGGAAAGAAATGCATAAACTTTTGCCAT
GGCCTGTTGAACAAGTCTGGAAAGAAATGCATAAACTTTTGCCAT
TCTCACCGGATTCAGTCGTCACTCATGGTGATTTCTCACTTGATA
TCTCACCGGATTCAGTCGTCACTCATGGTGATTTCTCACTTGATA
ACCTTATTTTTGACGAGGGGAAATTAATAGGTTGTATTGATGTTG
ACCTTATTTTTGACGAGGGGAAATTAATAGGTTGTATTGATGTTG
GACGAGTCGGAATCGCAGACCGATACCAGGATCTTGCCATCCTAT
GACGAGTCGGAATCGCAGACCGATACCAGGATCTTGCCATCCTAT
GGAACTGCCTCGGTGAGTTTTCTCCTTCATTACAGAAACGGCTTT
GGAACTGCCTCGGTGAGTTTTCTCCTTCATTACAGAAACGGCTTT
TTCAAAAATATGGTATTGATAATCCTGATATGAATAAATTGCAGT
TTCAAAAATATGGTATTGATAATCCTGATATGAATAAATTGCAGT
TTCATTTGATGCTCGATGAGTTTTTCTAATCTCATGACCAAAATC
TTCATTTGATGCTCGATGAGTTTTTCTAATCTCATGACCAAAATC
CCTTAACGTGAGTTTTCGTTCCACTGAGCGTCAGACCCCGTAGAA
CCTTAACGTGAGTTTTCGTTCCACTGAGCGTCAGACCCCGTAGAA
AAGATCAAAGGATCTTCTTGAGATCCTTTTTTTCTGCGCGTAATC
AAGATCAAAGGATCTTCTTGAGATCCTTTTTTTCTGCGCGTAATC
TGCTGCTTGCAAACAAAAAAACCACCGCTACCAGCGGTGGTTTGT
TGCTGCTTGCAAACAAAAAAACCACCGCTACCAGCGGTGGTTTGT
TTGCCGGATCAAGAGCTACCAACTCTTTTTCCGAAGGTAACTGGC
TTGCCGGATCAAGAGCTACCAACTCTTTTTCCGAAGGTAACTGGC
TTCAGCAGAGCGCAGATACCAAATACTGTCCTTCTAGTGTAGCCG
TTCAGCAGAGCGCAGATACCAAATACTGTCCTTCTAGTGTAGCCG
TAGTTAGGCCACCACTTCAAGAACTCTGTAGCACCGCCTACATAC
TAGTTAGGCCACCACTTCAAGAACTCTGTAGCACCGCCTACATAC
CTCGCTCTGCTAATCCTGTTACCAGTGGCTGCTGCCAGTGGCGAT
CTCGCTCTGCTAATCCTGTTACCAGTGGCTGCTGCCAGTGGCGAT
AAGTCGTGTCTTACCGGGTTGGACTCAAGACGATAGTTACCGGAT
AAGTCGTGTCTTACCGGGTTGGACTCAAGACGATAGTTACCGGAT
AAGGCGCAGCGGTCGGGCTGAACGGGGGGTTCGTGCACACAGCCC
AAGGCGCAGCGGTCGGGCTGAACGGGGGGTTCGTGCACACAGCCC
AGCTTGGAGCGAACGACCTACACCGAACTGAGATACCTACAGCGT
AGCTTGGAGCGAACGACCTACACCGAACTGAGATACCTACAGCGT
GAGCTATGAGAAAGCGCCACGCTTCCCGAAGGGAGAAAGGCGGAC
GAGCTATGAGAAAGCGCCACGCTTCCCGAAGGGAGAAAGGCGGAC
AGGTATCCGGTAAGCGGCAGGGTCGGAACAGGAGAGCGCACGAGG
AGGTATCCGGTAAGCGGCAGGGTCGGAACAGGAGAGCGCACGAGG
GAGCTTCCAGGGGGAAACGCCTGGTATCTTTATAGTCCTGTCGGG
GAGCTTCCAGGGGGAAACGCCTGGTATCTTTATAGTCCTGTCGGG
TTTCGCCACCTCTGACTTGAGCGTCGATTTTTGTGATGCTCGTCA
TTTCGCCACCTCTGACTTGAGCGTCGATTTTTGTGATGCTCGTCA
GGGGGGCGGAGCCTATGGAAAAACGCCAGCAACGCGGCCTTTTTA
GGGGGGCGGAGCCTATGGAAAAACGCCAGCAACGCGGCCTTTTTA
CGGTTCCTGGCCTTTTGCTGGCCTTTTGCTCACATGT

TABLE 4
Components of Construct Sequence (SEQ ID NO: 61)

| Components | Position in construct |
|---|---|
| 5′ITR | 12-130 |
| Cloning site | 131-147 |
| GDF6 promoter | 148-1335 |
| hGJB2 minimal promoter | 1336-1463 |
| Cloning site | 1464-1472 |
| Synthetic barcode | 1473-1480 |
| 5′UTR | 1481-1842 |
| GJB2 (exon2) | 1854-2531 |
| 3xFLAG | 2544-2609 |
| 3′UTR (exon2) | 2613-4019 |
| bGHpA | 4041-4265 |
| Cloning site | 4266-4299 |
| 3′ITR | 4300-4429 |

Exemplary Construct sequence
(SEQ ID NO: 54)
CCTGCAGGCAGCTGCGCGCTCGCTCGCTCACTGAGGCCGCCCGGG
CCTGCAGGCAGCTGCGCGCTCGCTCGCTCACTGAGGCCGCCCGGG
CGTCGGGCGACCTTTGGTCGCCCGGCCTCAGTGAGCGAGCGAGCG
CGTCGGGCGACCTTTGGTCGCCCGGCCTCAGTGAGCGAGCGAGCG
CGCAGAGAGGGAGTGGCCAACTCCATCACTAGGGGTTCCTGCGGC
CGCAGAGAGGGAGTGGCCAACTCCATCACTAGGGGTTCCTGCGGC
CGCACGCGTGGTAAGAAACTTGCCCGAGTTTACACAGCTAGTAAA
CGCACGCGTGGTAAGAAACTTGCCCGAGTTTACACAGCTAGTAAA
TGGTTGCATTAGTCAGGACAGCTAGCCTATATTACAATAACAACC
TGGTTGCATTAGTCAGGACAGCTAGCCTATATTACAATAACAACC
CTCTCAAATCCTAATGGCTTAAAACAACAGAGGTTTAATTTATAC
CTCTCAAATCCTAATGGCTTAAAACAACAGAGGTTTAATTTATAC
TCATTAGCTGTTCAAGGCAGGAGGCTCTATTCTCTAATCCATACA
TCATTAGCTGTTCAAGGCAGGAGGCTCTATTCTCTAATCCATACA
GTCACTCAGGATCCAGGCTGGTGGAGACCCTGCCATATTGTAGCC
GTCACTCAGGATCCAGGCTGGTGGAGACCCTGCCATATTGTAGCC
TCACCATTTAAAACATGAAGAAGATAGAAAGTGAGGAGTCATGTA
TCACCATTTAAAACATGAAGAAGATAGAAAGTGAGGAGTCATGTA
GGTTTTGTTCCGTTGCCTCAGGCTAGGAGTGACAGGTCACTTCAT
GGTTTTGTTCCGTTGCCTCAGGCTAGGAGTGACAGGTCACTTCAT
CTCACTCACAGCTCACTGCCCACAACTAGTCACTTGTGACTGTGC
CTCACTCACAGCTCACTGCCCACAACTAGTCACTTGTGACTGTGC
GAGTTAAGCTTCTGTGTGTGAAGGAAGGAAAAGAGAATGGGATAA
GAGTTAAGCTTCTGTGTGTGAAGGAAGGAAAAGAGAATGGGATAA
AGGTGAACATCAGCAGGCTCTACCACAGTAGTTTGAACCAAGACT
AGGTGAACATCAGCAGGCTCTACCACAGTAGTTTGAACCAAGACT
TGAGCCTAGGTCATGTGGCTTCAGAATCTTTGCTCTTAATCACAC
TGAGCCTAGGTCATGTGGCTTCAGAATCTTTGCTCTTAATCACAC
TAAACAGCCTCTGTAAGTCATCTTTCCTTCATCCAGTGCCTAAGA
TAAACAGCCTCTGTAAGTCATCTTTCCTTCATCCAGTGCCTAAGA
ACATGCAGTCCAATGCCCTCATCCTTCAGAAGAACTTGAGTGAAC
ACATGCAGTCCAATGCCCTCATCCTTCAGAAGAACTTGAGTGAAC
TCAGAGAAATTGAGTAGAGTGCCACAGCATGCCCAAGGCCACACA
TCAGAGAAATTGAGTAGAGTGCCACAGCATGCCCAAGGCCACACA
CCCTGAGGTTGGCAGTAGGTCCTGAGTTAGAGTTGTCATTTCTTG
CCCTGAGGTTGGCAGTAGGTCCTGAGTTAGAGTTGTCATTTCTTG
GCTCCCCTGGTAGTAGTGGAAAGGTAAGGTTTTGACATACTAGTT
GCTCCCCTGGTAGTAGTGGAAAGGTAAGGTTTTGACATACTAGTT
GGATGACCACGGGCAGGTCACTTAAATTGTCTAAGCATCGTTTGA
GGATGACCACGGGCAGGTCACTTAAATTGTCTAAGCATCGTTTGA
CCCTTGTAAGAATTAAATGAAATAGCACCTGTAAAAGTGTCTGCA
CCCTTGTAAGAATTAAATGAAATAGCACCTGTAAAAGTGTCTGCA
CGGACTTACTGCTGTTAGTTTTGTTCCTTTCTTCCTGTTGTCACT
CGGACTTACTGCTGTTAGTTTTGTTCCTTTCTTCCTGTTGTCACT
GCACTTCCCTGCCTGTTACCCAGGCCATGCAGACCAGCCAGGCCT
GCACTTCCCTGCCTGTTACCCAGGCCATGCAGACCAGCCAGGCCT
TCGACTTACAGTGCGGATAAGATTCCAAATCTCCACGGCTGGTTT
TCGACTTACAGTGCGGATAAGATTCCAAATCTCCACGGCTGGTTT
CCATGCTTTCTTCCAGGCTTCTGAGGACCCTGTGCTCTGGTTTCT
CCATGCTTTCTTCCAGGCTTCTGAGGACCCTGTGCTCTGGTTTCT
TCTATTTCTTTTCTATTACTTTTCTGTTACTCTTGAGCACACTTG
TCTATTTCTTTTCTATTACTTTTCTGTTACTCTTGAGCACACTTG
CTGGAAGCAATATGCATCCAGTTCTCCCTCTCTTGCCTCATTACA
CTGGAAGCAATATGCATCCAGTTCTCCCTCTCTTGCCTCATTACA
CTTTGCAGAACAACTCCAATCCCTTCCAACCAAGTAGTCCCTTTG
CTTTGCAGAACAACTCCAATCCCTTCCAACCAAGTAGTCCCTTTG
AATTTCTTGTCACCCAAGGAATCTCTCTGACAGGGGTCTTTGTTA
AATTTCTTGTCACCCAAGGAATCTCTCTGACAGGGGTCTTTGTTA
GGGTCACACCCCAGGAGATGGTTGATTATGGCTGAGTCCAGCCTG
GGGTCACACCCCAGGAGATGGTTGATTATGGCTGAGTCCAGCCTG
GAATGATGGGGGTTGGGGGCAGCTTGGGTAGATGACTCAGTAAAT
GAATGATGGGGGTTGGGGGCAGCTTGGGTAGATGACTCAGTAAAT
CAAACAGAACAATGAAAGGAGGTCATGCTTGTCCATCTGCATTAT
CAAACAGAACAATGAAAGGAGGTCATGCTTGTCCATCTGCATTAT
TGAAGACAGCCATAAATGGCCTTACCCCAGAGCGGGTCTGTCACA
TGAAGACAGCCATAAATGGCCTTACCCCAGAGCGGGTCTGTCACA
CCTGGAGAGCTGATCTGACCTCTCCAAGACCCCTGCAACTGAGTG
CCTGGAGAGCTGATCTGACCTCTCCAAGACCCCTGCAACTGAGTG
TTCTGGGATCTGTCCTGCAACAAGTGCCTCGAGATTTGTAGGTGG
TTCTGGGATCTGTCCTGCAACAAGTGCCTCGAGATTTGTAGGTGG
GGGCCCAGAGGGAGGGGGTCTGCAGACGAAGGGGGCAGGTTTTGC
GGGCCCAGAGGGAGGGGGTCTGCAGACGAAGGGGGCAGGTTTTGC
GGGGCACTTAGGGTTCTCATAGGTTGTAGTCACGAGCTCCAAGCT
GGGGCACTTAGGGTTCTCATAGGTTGTAGTCACGAGCTCCAAGCT
CTGAGGACCCAGAGGCCGGGCGCGCTCCGCCCGCGGCGCCGCCCC
CTGAGGACCCAGAGGCCGGGCGCGCTCCGCCCGCGGCGCCGCCCC
CTCCGTAACTTTCCCAGTCTCCGAGGGAAGAGGCGGGGTGTGGGG
CTCCGTAACTTTCCCAGTCTCCGAGGGAAGAGGCGGGGTGTGGGG
TGCGGTTAAAAGGCGCCACGGCGGGAGACAGGTCTCACCGGTCAC
TGCGGTTAAAAGGCGCCACGGCGGGAGACAGGTCTCACCGGTCAC
AACCTGTTGCGGCCCCGCAGCGCCCGCGCGCTCCTCTCCCCGACT
AACCTGTTGCGGCCCCGCAGCGCCCGCGCGCTCCTCTCCCCGACT
CGGAGCCCCTCGGCGGCGCCCGGCCCAGGACCCGCCTAGGAGCGC
CGGAGCCCCTCGGCGGCGCCCGGCCCAGGACCCGCCTAGGAGCGC
AGGAGCCCCAGCGCAGAGACCCCAACGCCGAGACCCCCGCCCCGG
AGGAGCCCCAGCGCAGAGACCCCAACGCCGAGACCCCCGCCCCGG
CCCCGCCGCGCTTCCTCCCGACGCAGTTTAGGACCCTTGTTCGCG
CCCCGCCGCGCTTCCTCCCGACGCAGTTTAGGACCCTTGTTCGCG
AAGAGGTGGTGTGCGGCTGAGACCCGCGTCCTCAGGACGGTTCCA
AAGAGGTGGTGTGCGGCTGAGACCCGCGTCCTCAGGACGGTTCCA
TCAGTGCCTCGATCCTGCCCCACTGGAGGAGGAAGGCAGCCCGAA
TCAGTGCCTCGATCCTGCCCCACTGGAGGAGGAAGGCAGCCCGAA
CAGCGCTCACCTAACTAACAGCTGCTGAGAGCTGGGTTCCGTGGC
CAGCGCTCACCTAACTAACAGCTGCTGAGAGCTGGGTTCCGTGGC
CATGCACCTGGGACTGCCTTGAGAAGCGTGAGCAAACCGCCCAGA
CATGCACCTGGGACTGCCTTGAGAAGCGTGAGCAAACCGCCCAGA
GTAGAAGCGCTAGCCACCATGGATTGGGGCACGCTGCAGACGATC
GTAGAAGCGCTAGCCACCATGGATTGGGGCACGCTGCAGACGATC
CTGGGGGGTGTGAACAAACACTCCACCAGCATTGGAAAGATCTGG
CTGGGGGGTGTGAACAAACACTCCACCAGCATTGGAAAGATCTGG
CTCACCGTCCTCTTCATTTTTCGCATTATGATCCTCGTTGTGGCT
CTCACCGTCCTCTTCATTTTTCGCATTATGATCCTCGTTGTGGCT
GCAAAGGAGGTGTGGGGAGATGAGCAGGCCGACTTTGTCTGCAAC
GCAAAGGAGGTGTGGGGAGATGAGCAGGCCGACTTTGTCTGCAAC
ACCCTGCAGCCAGGCTGCAAGAACGTGTGCTACGATCACTACTTC
ACCCTGCAGCCAGGCTGCAAGAACGTGTGCTACGATCACTACTTC
CCCATCTCCCACATCCGGCTATGGGCCCTGCAGCTGATCTTCGTG
CCCATCTCCCACATCCGGCTATGGGCCCTGCAGCTGATCTTCGTG
TCCACGCCAGCGCTCCTAGTGGCCATGCACGTGGCCTACCGGAGA
TCCACGCCAGCGCTCCTAGTGGCCATGCACGTGGCCTACCGGAGA
CATGAGAAGAAGAGGAAGTTCATCAAGGGGGAGATAAAGAGTGAA
CATGAGAAGAAGAGGAAGTTCATCAAGGGGGAGATAAAGAGTGAA
TTTAAGGACATCGAGGAGATCAAAACCCAGAAGGTCCGCATCGAA
TTTAAGGACATCGAGGAGATCAAAACCCAGAAGGTCCGCATCGAA
GGCTCCCTGTGGTGGACCTACACAAGCAGCATCTTCTTCCGGGTC
GGCTCCCTGTGGTGGACCTACACAAGCAGCATCTTCTTCCGGGTC
ATCTTCGAAGCCGCCTTCATGTACGTCTTCTATGTCATGTACGAC
ATCTTCGAAGCCGCCTTCATGTACGTCTTCTATGTCATGTACGAC
GGCTTCTCCATGCAGCGGCTGGTGAAGTGCAACGCCTGGCCTTGT
GGCTTCTCCATGCAGCGGCTGGTGAAGTGCAACGCCTGGCCTTGT
CCCAACACTGTGGACTGCTTTGTGTCCCGGCCCACGGAGAAGACT
CCCAACACTGTGGACTGCTTTGTGTCCCGGCCCACGGAGAAGACT
GTCTTCACAGTGTTCATGATTGCAGTGTCTGGAATTTGCATCCTG
GTCTTCACAGTGTTCATGATTGCAGTGTCTGGAATTTGCATCCTG
CTGAATGTCACTGAATTGTGTTATTTGCTAATTAGATATTGTTCT
CTGAATGTCACTGAATTGTGTTATTTGCTAATTAGATATTGTTCT
GGGAAGTCAAAAAAGCCAGTTGGATCCCGGGCTGACTACAAAGAC
GGGAAGTCAAAAAAGCCAGTTGGATCCCGGGCTGACTACAAAGAC
CATGACGGTGATTATAAAGATCATGACATCGACTACAAGGATGAC
CATGACGGTGATTATAAAGATCATGACATCGACTACAAGGATGAC
GATGACAAGTAAGAAATAGACAGCATGAGAGGGATGAGGCAACCC
GATGACAAGTAAGAAATAGACAGCATGAGAGGGATGAGGCAACCC
GTGCTCAGCTGTCAAGGCTCAGTCGCTAGCATTTCCCAACACAAA
GTGCTCAGCTGTCAAGGCTCAGTCGCTAGCATTTCCCAACACAAA
GATTCTGACCTTAAATGCAACCATTTGAAACCCCTGTAGGCCTCA
GATTCTGACCTTAAATGCAACCATTTGAAACCCCTGTAGGCCTCA
GGTGAAACTCCAGATGCCACAATGGAGCTCTGCTCCCCTAAAGCC
GGTGAAACTCCAGATGCCACAATGGAGCTCTGCTCCCCTAAAGCC
TCAAAACAAAGGCCTAATTCTATGCCTGTCTTAATTTTCTTTCAC
TCAAAACAAAGGCCTAATTCTATGCCTGTCTTAATTTTCTTTCAC
TTAAGTTAGTTCCACTGAGACCCCAGGCTGTTAGGGGTTATTGGT
TTAAGTTAGTTCCACTGAGACCCCAGGCTGTTAGGGGTTATTGGT
GTAAGGTACTTTCATATTTTAAACAGAGGATATCGGCATTTGTTT
GTAAGGTACTTTCATATTTTAAACAGAGGATATCGGCATTTGTTT
CTTTCTCTGAGGACAAGAGAAAAAAGCCAGGTTCCACAGAGGACA
CTTTCTCTGAGGACAAGAGAAAAAAGCCAGGTTCCACAGAGGACA
CAGAGAAGGTTTGGGTGTCCTCCTGGGGTTCTTTTTGCCAACTTT
CAGAGAAGGTTTGGGTGTCCTCCTGGGGTTCTTTTTGCCAACTTT
CCCCACGTTAAAGGTGAACATTGGTTCTTTCATTTGCTTTGGAAG
CCCCACGTTAAAGGTGAACATTGGTTCTTTCATTTGCTTTGGAAG
TTTTAATCTCTAACAGTGGACAAAGTTACCAGTGCCTTAAACTCT
TTTTAATCTCTAACAGTGGACAAAGTTACCAGTGCCTTAAACTCT
GTTACACTTTTTGGAAGTGAAAACTTTGTAGTATGATAGGTTATT
GTTACACTTTTTGGAAGTGAAAACTTTGTAGTATGATAGGTTATT
TTGATGTAAAGATGTTCTGGATACCATTATATGTTCCCCCTGTTT
TTGATGTAAAGATGTTCTGGATACCATTATATGTTCCCCCTGTTT
CAGAGGCTCAGATTGTAATATGTAAATGGTATGTCATTCGCTACT
CAGAGGCTCAGATTGTAATATGTAAATGGTATGTCATTCGCTACT
ATGATTTAATTTGAAATATGGTCTTTTGGTTATGAATACTTTGCA
ATGATTTAATTTGAAATATGGTCTTTTGGTTATGAATACTTTGCA
GCACAGCTGAGAGGCTGTCTGTTGTATTCATTGTGGTCATAGCAC
GCACAGCTGAGAGGCTGTCTGTTGTATTCATTGTGGTCATAGCAC
CTAACAACATTGTAGCCTCAATCGAGTGAGACAGACTAGAAGTTC
CTAACAACATTGTAGCCTCAATCGAGTGAGACAGACTAGAAGTTC
CTAGTGATGGCTTATGATAGCAAATGGCCTCATGTCAAATATTTA
CTAGTGATGGCTTATGATAGCAAATGGCCTCATGTCAAATATTTA
GATGTAATTTTGTGTAAGAAATACAGACTGGATGTACCACCAACT
GATGTAATTTTGTGTAAGAAATACAGACTGGATGTACCACCAACT
ACTACCTGTAATGACAGGCCTGTCCAACACATCTCCCTTTTCCAT
ACTACCTGTAATGACAGGCCTGTCCAACACATCTCCCTTTTCCAT
GACTGTGGTAGCCAGCATCGGAAAGAACGCTGATTTAAAGAGGTC
GACTGTGGTAGCCAGCATCGGAAAGAACGCTGATTTAAAGAGGTC
GCTTGGGAATTTTATTGACACAGTACCATTTAATGGGGAGGACAA
GCTTGGGAATTTTATTGACACAGTACCATTTAATGGGGAGGACAA
AATGGGGCAGGGGAGGGAGAAGTTTCTGTCGTTAAAAACAGATTT
AATGGGGCAGGGGAGGGAGAAGTTTCTGTCGTTAAAAACAGATTT
GGAAAGACTGGACTCTAAAGTCTGTTGATTAAAGATGAGCTTTGT
GGAAAGACTGGACTCTAAAGTCTGTTGATTAAAGATGAGCTTTGT
CTACTTCAAAAGTTTGTTTGCTTACCCCTTCAGCCTCCAATTTTT
CTACTTCAAAAGTTTGTTTGCTTACCCCTTCAGCCTCCAATTTTT
TAAGTGAAAATATAGCTAATAACATGTGAAAAGAATAGAAGCTAA
TAAGTGAAAATATAGCTAATAACATGTGAAAAGAATAGAAGCTAA
GGTTTAGATAAATATTGAGCAGATCTATAGGAAGATTGAACCTGA
GGTTTAGATAAATATTGAGCAGATCTATAGGAAGATTGAACCTGA
ATATTGCCATTATGCTTGACATGGTTTCCAAAAAATGGTACTCCA
ATATTGCCATTATGCTTGACATGGTTTCCAAAAAATGGTACTCCA
CATATTTCAGTGAGGGTAAGTATTTTCCTGTTGTCAAGAATAGCA
CATATTTCAGTGAGGGTAAGTATTTTCCTGTTGTCAAGAATAGCA
TTGTAAAAGCATTTTGTAATAATAAAGAATAGCTTTAATGATATG
TTGTAAAAGCATTTTGTAATAATAAAGAATAGCTTTAATGATATG
CTTGTAACTAAAATAATTTTGTAATGTATCAAATACATTTAAAAC
CTTGTAACTAAAATAATTTTGTAATGTATCAAATACATTTAAAAC
ATTAAAATATAATCTCTATAATAAGAGCTCGCTGATCAGCCTCGA
ATTAAAATATAATCTCTATAATAAGAGCTCGCTGATCAGCCTCGA
CTGTGCCTTCTAGTTGCCAGCCATCTGTTGTTTGCCCCTCCCCCG
CTGTGCCTTCTAGTTGCCAGCCATCTGTTGTTTGCCCCTCCCCCG
TGCCTTCCTTGACCCTGGAAGGTGCCACTCCCACTGTCCTTTCCT
TGCCTTCCTTGACCCTGGAAGGTGCCACTCCCACTGTCCTTTCCT
AATAAAATGAGGAAATTGCATCGCATTGTCTGAGTAGGTGTCATT
AATAAAATGAGGAAATTGCATCGCATTGTCTGAGTAGGTGTCATT
CTATTCTGGGGGGTGGGGTGGGGCAGGACAGCAAGGGGGAGGATT
CTATTCTGGGGGGTGGGGTGGGGCAGGACAGCAAGGGGGAGGATT
GGGAAGACAATAGCAGGCATGCTGGGGATGCGGTGGGCTCTATGG
GGGAAGACAATAGCAGGCATGCTGGGGATGCGGTGGGCTCTATGG
AAGCTTGAATTCAGCTGACGTGCCTCGGACCGCTAGGAACCCCTA
AAGCTTGAATTCAGCTGACGTGCCTCGGACCGCTAGGAACCCCTA
GTGATGGAGTTGGCCACTCCCTCTCTGCGCGCTCGCTCGCTCACT
GTGATGGAGTTGGCCACTCCCTCTCTGCGCGCTCGCTCGCTCACT
GAGGCCGGGCGACCAAAGGTCGCCCGACGCCCGGGCTTTGCCCGG
GAGGCCGGGCGACCAAAGGTCGCCCGACGCCCGGGCTTTGCCCGG
GCGGCCTCAGTGAGCGAGCGAGCGCGCAGCTGCCTGCAGGGGCGC
GCGGCCTCAGTGAGCGAGCGAGCGCGCAGCTGCCTGCAGGGGCGC
CTGATGCGGTATTTTCTCCTTACGCATCTGTGCGGTATTTCACAC
CTGATGCGGTATTTTCTCCTTACGCATCTGTGCGGTATTTCACAC
CGCATACGTCAAAGCAACCATAGTACGCGCCCTGTAGCGGCGCAT
CGCATACGTCAAAGCAACCATAGTACGCGCCCTGTAGCGGCGCAT
TAAGCGCGGCGGGTGTGGTGGTTACGCGCAGCGTGACCGCTACAC
TAAGCGCGGCGGGTGTGGTGGTTACGCGCAGCGTGACCGCTACAC
TTGCCAGCGCCCTAGCGCCCGCTCCTTTCGCTTTCTTCCCTTCCT
TTGCCAGCGCCCTAGCGCCCGCTCCTTTCGCTTTCTTCCCTTCCT
TTCTCGCCACGTTCGCCGGCTTTCCCCGTCAAGCTCTAAATCGGG
TTCTCGCCACGTTCGCCGGCTTTCCCCGTCAAGCTCTAAATCGGG
GGCTCCCTTTAGGGTTCCGATTTAGTGCTTTACGGCACCTCGACC
GGCTCCCTTTAGGGTTCCGATTTAGTGCTTTACGGCACCTCGACC
CCAAAAAACTTGATTTGGGTGATGGTTCACGTAGTGGGCCATCGC
CCAAAAAACTTGATTTGGGTGATGGTTCACGTAGTGGGCCATCGC
CCTGATAGACGGTTTTTCGCCCTTTGACGTTGGAGTCCACGTTCT
CCTGATAGACGGTTTTTCGCCCTTTGACGTTGGAGTCCACGTTCT
TTAATAGTGGACTCTTGTTCCAAACTGGAACAACACTCAACCCTA
TTAATAGTGGACTCTTGTTCCAAACTGGAACAACACTCAACCCTA
TCTCGGGCTATTCTTTTGATTTATAAGGGATTTTGCCGATTTCGG
TCTCGGGCTATTCTTTTGATTTATAAGGGATTTTGCCGATTTCGG
CCTATTGGTTAAAAAATGAGCTGATTTAACAAAAATTTAACGCGA
CCTATTGGTTAAAAAATGAGCTGATTTAACAAAAATTTAACGCGA
ATTTTAACAAAATATTAACGTTTACAATTTTATGGTGCACTCTCA
ATTTTAACAAAATATTAACGTTTACAATTTTATGGTGCACTCTCA
GTACAATCTGCTCTGATGCCGCATAGTTAAGCCAGCCCCGACACC
GTACAATCTGCTCTGATGCCGCATAGTTAAGCCAGCCCCGACACC
CGCCAACACCCGCTGACGCGCCCTGACGGGCTTGTCTGCTCCCGG
CGCCAACACCCGCTGACGCGCCCTGACGGGCTTGTCTGCTCCCGG
CATCCGCTTACAGACAAGCTGTGACCGTCTCCGGGAGCTGCATGT
CATCCGCTTACAGACAAGCTGTGACCGTCTCCGGGAGCTGCATGT
GTCAGAGGTTTTCACCGTCATCACCGAAACGCGCGAGACGAAAGG
GTCAGAGGTTTTCACCGTCATCACCGAAACGCGCGAGACGAAAGG
GCCTCGTGATACGCCTATTTTTATAGGTTAATGTCATGAACAATA
GCCTCGTGATACGCCTATTTTTATAGGTTAATGTCATGAACAATA
AAACTGTCTGCTTACATAAACAGTAATACAAGGGGTGTTATGAGC
AAACTGTCTGCTTACATAAACAGTAATACAAGGGGTGTTATGAGC
CATATTCAACGGGAAACGTCGAGGCCGCGATTAAATTCCAACATG
CATATTCAACGGGAAACGTCGAGGCCGCGATTAAATTCCAACATG
GATGCTGATTTATATGGGTATAAATGGGCTCGCGATAATGTCGGG
GATGCTGATTTATATGGGTATAAATGGGCTCGCGATAATGTCGGG
CAATCAGGTGCGACAATCTATCGCTTGTATGGGAAGCCCGATGCG
CAATCAGGTGCGACAATCTATCGCTTGTATGGGAAGCCCGATGCG
CCAGAGTTGTTTCTGAAACATGGCAAAGGTAGCGTTGCCAATGAT
CCAGAGTTGTTTCTGAAACATGGCAAAGGTAGCGTTGCCAATGAT
GTTACAGATGAGATGGTCAGACTAAACTGGCTGACGGAATTTATG
GTTACAGATGAGATGGTCAGACTAAACTGGCTGACGGAATTTATG
CCTCTTCCGACCATCAAGCATTTTATCCGTACTCCTGATGATGCA
CCTCTTCCGACCATCAAGCATTTTATCCGTACTCCTGATGATGCA
TGGTTACTCACCACTGCGATCCCCGGAAAAACAGCATTCCAGGTA
TGGTTACTCACCACTGCGATCCCCGGAAAAACAGCATTCCAGGTA
TTAGAAGAATATCCTGATTCAGGTGAAAATATTGTTGATGCGCTG
TTAGAAGAATATCCTGATTCAGGTGAAAATATTGTTGATGCGCTG
GCAGTGTTCCTGCGCCGGTTGCATTCGATTCCTGTTTGTAATTGT
GCAGTGTTCCTGCGCCGGTTGCATTCGATTCCTGTTTGTAATTGT
CCTTTTAACAGCGATCGCGTATTTCGTCTCGCTCAGGCGCAATCA
CCTTTTAACAGCGATCGCGTATTTCGTCTCGCTCAGGCGCAATCA
CGAATGAATAACGGTTTGGTTGATGCGAGTGATTTTGATGACGAG
CGAATGAATAACGGTTTGGTTGATGCGAGTGATTTTGATGACGAG
CGTAATGGCTGGCCTGTTGAACAAGTCTGGAAAGAAATGCATAAA
CGTAATGGCTGGCCTGTTGAACAAGTCTGGAAAGAAATGCATAAA
CTTTTGCCATTCTCACCGGATTCAGTCGTCACTCATGGTGATTTC
CTTTTGCCATTCTCACCGGATTCAGTCGTCACTCATGGTGATTTC
TCACTTGATAACCTTATTTTTGACGAGGGGAAATTAATAGGTTGT
TCACTTGATAACCTTATTTTTGACGAGGGGAAATTAATAGGTTGT
ATTGATGTTGGACGAGTCGGAATCGCAGACCGATACCAGGATCTT
ATTGATGTTGGACGAGTCGGAATCGCAGACCGATACCAGGATCTT
GCCATCCTATGGAACTGCCTCGGTGAGTTTTCTCCTTCATTACAG
GCCATCCTATGGAACTGCCTCGGTGAGTTTTCTCCTTCATTACAG
AAACGGCTTTTTCAAAAATATGGTATTGATAATCCTGATATGAAT
AAACGGCTTTTTCAAAAATATGGTATTGATAATCCTGATATGAAT
AAATTGCAGTTTCATTTGATGCTCGATGAGTTTTTCTAATCTCAT
AAATTGCAGTTTCATTTGATGCTCGATGAGTTTTTCTAATCTCAT
GACCAAAATCCCTTAACGTGAGTTTTCGTTCCACTGAGCGTCAGA
GACCAAAATCCCTTAACGTGAGTTTTCGTTCCACTGAGCGTCAGA
CCCCGTAGAAAAGATCAAAGGATCTTCTTGAGATCCTTTTTTTCT
CCCCGTAGAAAAGATCAAAGGATCTTCTTGAGATCCTTTTTTTCT
GCGCGTAATCTGCTGCTTGCAAACAAAAAAACCACCGCTACCAGC
GCGCGTAATCTGCTGCTTGCAAACAAAAAAACCACCGCTACCAGC
GGTGGTTTGTTTGCCGGATCAAGAGCTACCAACTCTTTTTCCGAA
GGTGGTTTGTTTGCCGGATCAAGAGCTACCAACTCTTTTTCCGAA
GGTAACTGGCTTCAGCAGAGCGCAGATACCAAATACTGTCCTTCT
GGTAACTGGCTTCAGCAGAGCGCAGATACCAAATACTGTCCTTCT
AGTGTAGCCGTAGTTAGGCCACCACTTCAAGAACTCTGTAGCACC
AGTGTAGCCGTAGTTAGGCCACCACTTCAAGAACTCTGTAGCACC
GCCTACATACCTCGCTCTGCTAATCCTGTTACCAGTGGCTGCTGC
GCCTACATACCTCGCTCTGCTAATCCTGTTACCAGTGGCTGCTGC
CAGTGGCGATAAGTCGTGTCTTACCGGGTTGGACTCAAGACGATA
CAGTGGCGATAAGTCGTGTCTTACCGGGTTGGACTCAAGACGATA
GTTACCGGATAAGGCGCAGCGGTCGGGCTGAACGGGGGGTTCGTG
GTTACCGGATAAGGCGCAGCGGTCGGGCTGAACGGGGGGTTCGTG
CACACAGCCCAGCTTGGAGCGAACGACCTACACCGAACTGAGATA
CACACAGCCCAGCTTGGAGCGAACGACCTACACCGAACTGAGATA
CCTACAGCGTGAGCTATGAGAAAGCGCCACGCTTCCCGAAGGGAG
CCTACAGCGTGAGCTATGAGAAAGCGCCACGCTTCCCGAAGGGAG
AAAGGCGGACAGGTATCCGGTAAGCGGCAGGGTCGGAACAGGAGA
AAAGGCGGACAGGTATCCGGTAAGCGGCAGGGTCGGAACAGGAGA
GCGCACGAGGGAGCTTCCAGGGGGAAACGCCTGGTATCTTTATAG
GCGCACGAGGGAGCTTCCAGGGGGAAACGCCTGGTATCTTTATAG
TCCTGTCGGGTTTCGCCACCTCTGACTTGAGCGTCGATTTTTGTG
TCCTGTCGGGTTTCGCCACCTCTGACTTGAGCGTCGATTTTTGTG
ATGCTCGTCAGGGGGGCGGAGCCTATGGAAAAACGCCAGCAACGC
ATGCTCGTCAGGGGGGCGGAGCCTATGGAAAAACGCCAGCAACGC
GGCCTTTTTACGGTTCCTGGCCTTTTGCTGGCCTTTTGCTCACAT
GGCCTTTTTACGGTTCCTGGCCTTTTGCTGGCCTTTTGCTCACAT
GT

TABLE 5
Components of Construct Sequence (SEQ ID NO: 54)

| Components | Position in construct |
|---|---|
| 5′ITR | 12-130 |
| Cloning site | 131-147 |
| IGFBP2 promoter | 148-1660 |
| hGJB2 minimal promoter | 1661-1788 |
| Cloning site | 1789-1797 |
| Synthetic barcode | 1798-1805 |
| 5′UTR | 1806-2167 |
| GJB2 (exon2) | 2179-2856 |
| 3xFLAG | 2869-2934 |
| 3′UTR (exon2) | 2938-4344 |
| bGHpA | 4366-4590 |
| Cloning site | 4591-4624 |
| 3′ITR | 4625-4754 |

Exemplary Construct sequence
(SEQ ID NO: 17)
CCTGCAGGCAGCTGCGCGCTCGCTCGCTCACTGAGGCCGCCCGGG
CCTGCAGGCAGCTGCGCGCTCGCTCGCTCACTGAGGCCGCCCGGG
CGTCGGGCGACCTTTGGTCGCCCGGCCTCAGTGAGCGAGCGAGCG
CGTCGGGCGACCTTTGGTCGCCCGGCCTCAGTGAGCGAGCGAGCG
CGCAGAGAGGGAGTGGCCAACTCCATCACTAGGGGTTCCTGCGGC
CGCAGAGAGGGAGTGGCCAACTCCATCACTAGGGGTTCCTGCGGC
CGCACGCGTGGTCCCATGGCTCTGTTAAAATCAAAGAAACATCTT
CGCACGCGTGGTCCCATGGCTCTGTTAAAATCAAAGAAACATCTT
TTCCAACAGCCCTTTCAAACTCCTCATCGCATCTCACTGGCTGAT
TTCCAACAGCCCTTTCAAACTCCTCATCGCATCTCACTGGCTGAT
TCAGTCATTTAAACCTGCTTCTCCCTAAAGCTGATCACTGGCTAA
TCAGTCATTTAAACCTGCTTCTCCCTAAAGCTGATCACTGGCTAA
GCTAATAGGGTTTCCGGGATTGGTTTAGCCTGATACTAATCCAGG
GCTAATAGGGTTTCCGGGATTGGTTTAGCCTGATACTAATCCAGG
TCTACCTTCAGGAGCCAGACCAAACTGCCTATTGGCATTGCATTC
TCTACCTTCAGGAGCCAGACCAAACTGCCTATTGGCATTGCATTC
TTGCAGTAGGGAGGGGAGGTATGGATGGTGTGGAGTCCACCACAA
TTGCAGTAGGGAGGGGAGGTATGGATGGTGTGGAGTCCACCACAA
GGTCCATGCCAGTCTTTGCTGAACCAGCATCAGACTCCATCAAGC
GGTCCATGCCAGTCTTTGCTGAACCAGCATCAGACTCCATCAAGC
AACAGATGAGAGGTTCCATGATAAAGTGGCCCTCAGCAATCCCCA
AACAGATGAGAGGTTCCATGATAAAGTGGCCCTCAGCAATCCCCA
TCCATTGCTGTCTAGGAAGAACAGTGCTTGTACACAGGTTTAGGA
TCCATTGCTGTCTAGGAAGAACAGTGCTTGTACACAGGTTTAGGA
CCTCAGTCTTGGCTGTAATCTTCTGGTTTACTTTGCCAGCACCAA
CCTCAGTCTTGGCTGTAATCTTCTGGTTTACTTTGCCAGCACCAA
ACAGAAGGAAAGAAAGGGCTCAAATTTGACCAAATAAATTATGCT
ACAGAAGGAAAGAAAGGGCTCAAATTTGACCAAATAAATTATGCT
TCTCCTTCCAGAGATAACCTTGAGTCCTGTCTAGGAAGATATTAG
TCTCCTTCCAGAGATAACCTTGAGTCCTGTCTAGGAAGATATTAG
AATTGTAAAGAAAAAAAAAATTACTCCTTATCCTATGGCAAGTGG
AATTGTAAAGAAAAAAAAAATTACTCCTTATCCTATGGCAAGTGG
AGTCTATGTCTACTTCAGCTGAAATTAAATCCTGTCCATAATAGA
AGTCTATGTCTACTTCAGCTGAAATTAAATCCTGTCCATAATAGA
TGACCCTTGCTCAAGCTGGCCAGAAGCCATACCAACCAGCACGAA
TGACCCTTGCTCAAGCTGGCCAGAAGCCATACCAACCAGCACGAA
GGTTAAAACTATTATTAGTTTTTTCTGTGATTTTCATTTTCAGGC
GGTTAAAACTATTATTAGTTTTTTCTGTGATTTTCATTTTCAGGC
CAAGTTTTAGAACAATAAGATTTTAAGAATAGGAAGTAAGTAAGA
CAAGTTTTAGAACAATAAGATTTTAAGAATAGGAAGTAAGTAAGA
TTTCTGCATATCCTGTTCTCTTAGTCAGCTGAATTTTTTTTTTTT
TTTCTGCATATCCTGTTCTCTTAGTCAGCTGAATTTTTTTTTTTT
TTTTTTTAGTCCTAACTCAGCCTCCCAAAGTGCTGGGATTACAGG
TTTTTTTAGTCCTAACTCAGCCTCCCAAAGTGCTGGGATTACAGG
CGTGAGCCACCGCACCAAGCCTGGAATCTATGTCTTACAGTTATG
CGTGAGCCACCGCACCAAGCCTGGAATCTATGTCTTACAGTTATG
AGAATCAACAGCTAGCTCATTATGGGCAAGGTGATGTCACTCTGG
AGAATCAACAGCTAGCTCATTATGGGCAAGGTGATGTCACTCTGG
CTTCTCAATGAAAATGGCATTTCTCCCTTGGAAAAGGTCATAGCC
CTTCTCAATGAAAATGGCATTTCTCCCTTGGAAAAGGTCATAGCC
AGTCAGTCAGTCAGTCACGGGAGCGCAGCGGCTTCTAGGGGTGAG
AGTCAGTCAGTCAGTCACGGGAGCGCAGCGGCTTCTAGGGGTGAG
TGGGACCCACGCGGCCCCACCTGCTCCTCCCGCGCGCGGCCCCAC
TGGGACCCACGCGGCCCCACCTGCTCCTCCCGCGCGCGGCCCCAC
CCCCCTGCCCCGCCCCGCCTGGTTTATAGAAGCTCTGAGGACCCA
CCCCCTGCCCCGCCCCGCCTGGTTTATAGAAGCTCTGAGGACCCA
GAGGCCGGGCGCGCTCCGCCCGCGGCGCCGCCCCCTCCGTAACTT
GAGGCCGGGCGCGCTCCGCCCGCGGCGCCGCCCCCTCCGTAACTT
TCCCAGTCTCCGAGGGAAGAGGCGGGGTGTGGGGTGCGGTTAAAA
TCCCAGTCTCCGAGGGAAGAGGCGGGGTGTGGGGTGCGGTTAAAA
GGCGCCACGGCGGGAGACAGGTCTCACCGGTCGTGTGTTGTTGCG
GGCGCCACGGCGGGAGACAGGTCTCACCGGTCGTGTGTTGTTGCG
GCCCCGCAGCGCCCGCGCGCTCCTCTCCCCGACTCGGAGCCCCTC
GCCCCGCAGCGCCCGCGCGCTCCTCTCCCCGACTCGGAGCCCCTC
GGCGGCGCCCGGCCCAGGACCCGCCTAGGAGCGCAGGAGCCCCAG
GGCGGCGCCCGGCCCAGGACCCGCCTAGGAGCGCAGGAGCCCCAG
CGCAGAGACCCCAACGCCGAGACCCCCGCCCCGGCCCCGCCGCGC
CGCAGAGACCCCAACGCCGAGACCCCCGCCCCGGCCCCGCCGCGC
TTCCTCCCGACGCAGTTTAGGACCCTTGTTCGCGAAGAGGTGGTG
TTCCTCCCGACGCAGTTTAGGACCCTTGTTCGCGAAGAGGTGGTG
TGCGGCTGAGACCCGCGTCCTCAGGACGGTTCCATCAGTGCCTCG
TGCGGCTGAGACCCGCGTCCTCAGGACGGTTCCATCAGTGCCTCG
ATCCTGCCCCACTGGAGGAGGAAGGCAGCCCGAACAGCGCTCACC
ATCCTGCCCCACTGGAGGAGGAAGGCAGCCCGAACAGCGCTCACC
TAACTAACAGCTGCTGAGAGCTGGGTTCCGTGGCCATGCACCTGG
TAACTAACAGCTGCTGAGAGCTGGGTTCCGTGGCCATGCACCTGG
GACTGCCTTGAGAAGCGTGAGCAAACCGCCCAGAGTAGAAGCGCT
GACTGCCTTGAGAAGCGTGAGCAAACCGCCCAGAGTAGAAGCGCT
AGCCACCATGGATTGGGGCACGCTGCAGACGATCCTGGGGGGTGT
AGCCACCATGGATTGGGGCACGCTGCAGACGATCCTGGGGGGTGT
GAACAAACACTCCACCAGCATTGGAAAGATCTGGCTCACCGTCCT
GAACAAACACTCCACCAGCATTGGAAAGATCTGGCTCACCGTCCT
CTTCATTTTTCGCATTATGATCCTCGTTGTGGCTGCAAAGGAGGT
CTTCATTTTTCGCATTATGATCCTCGTTGTGGCTGCAAAGGAGGT
GTGGGGAGATGAGCAGGCCGACTTTGTCTGCAACACCCTGCAGCC
GTGGGGAGATGAGCAGGCCGACTTTGTCTGCAACACCCTGCAGCC
AGGCTGCAAGAACGTGTGCTACGATCACTACTTCCCCATCTCCCA
AGGCTGCAAGAACGTGTGCTACGATCACTACTTCCCCATCTCCCA
CATCCGGCTATGGGCCCTGCAGCTGATCTTCGTGTCCACGCCAGC
CATCCGGCTATGGGCCCTGCAGCTGATCTTCGTGTCCACGCCAGC
GCTCCTAGTGGCCATGCACGTGGCCTACCGGAGACATGAGAAGAA
GCTCCTAGTGGCCATGCACGTGGCCTACCGGAGACATGAGAAGAA
GAGGAAGTTCATCAAGGGGGAGATAAAGAGTGAATTTAAGGACAT
GAGGAAGTTCATCAAGGGGGAGATAAAGAGTGAATTTAAGGACAT
CGAGGAGATCAAAACCCAGAAGGTCCGCATCGAAGGCTCCCTGTG
CGAGGAGATCAAAACCCAGAAGGTCCGCATCGAAGGCTCCCTGTG
GTGGACCTACACAAGCAGCATCTTCTTCCGGGTCATCTTCGAAGC
GTGGACCTACACAAGCAGCATCTTCTTCCGGGTCATCTTCGAAGC
CGCCTTCATGTACGTCTTCTATGTCATGTACGACGGCTTCTCCAT
CGCCTTCATGTACGTCTTCTATGTCATGTACGACGGCTTCTCCAT
GCAGCGGCTGGTGAAGTGCAACGCCTGGCCTTGTCCCAACACTGT
GCAGCGGCTGGTGAAGTGCAACGCCTGGCCTTGTCCCAACACTGT
GGACTGCTTTGTGTCCCGGCCCACGGAGAAGACTGTCTTCACAGT
GGACTGCTTTGTGTCCCGGCCCACGGAGAAGACTGTCTTCACAGT
GTTCATGATTGCAGTGTCTGGAATTTGCATCCTGCTGAATGTCAC
GTTCATGATTGCAGTGTCTGGAATTTGCATCCTGCTGAATGTCAC
TGAATTGTGTTATTTGCTAATTAGATATTGTTCTGGGAAGTCAAA
TGAATTGTGTTATTTGCTAATTAGATATTGTTCTGGGAAGTCAAA
AAAGCCAGTTGGATCCCGGGCTGACTACAAAGACCATGACGGTGA
AAAGCCAGTTGGATCCCGGGCTGACTACAAAGACCATGACGGTGA
TTATAAAGATCATGACATCGACTACAAGGATGACGATGACAAGTA
TTATAAAGATCATGACATCGACTACAAGGATGACGATGACAAGTA
AGAAATAGACAGCATGAGAGGGATGAGGCAACCCGTGCTCAGCTG
AGAAATAGACAGCATGAGAGGGATGAGGCAACCCGTGCTCAGCTG
TCAAGGCTCAGTCGCTAGCATTTCCCAACACAAAGATTCTGACCT
TCAAGGCTCAGTCGCTAGCATTTCCCAACACAAAGATTCTGACCT
TAAATGCAACCATTTGAAACCCCTGTAGGCCTCAGGTGAAACTCC
TAAATGCAACCATTTGAAACCCCTGTAGGCCTCAGGTGAAACTCC
AGATGCCACAATGGAGCTCTGCTCCCCTAAAGCCTCAAAACAAAG
AGATGCCACAATGGAGCTCTGCTCCCCTAAAGCCTCAAAACAAAG
GCCTAATTCTATGCCTGTCTTAATTTTCTTTCACTTAAGTTAGTT
GCCTAATTCTATGCCTGTCTTAATTTTCTTTCACTTAAGTTAGTT
CCACTGAGACCCCAGGCTGTTAGGGGTTATTGGTGTAAGGTACTT
CCACTGAGACCCCAGGCTGTTAGGGGTTATTGGTGTAAGGTACTT
TCATATTTTAAACAGAGGATATCGGCATTTGTTTCTTTCTCTGAG
TCATATTTTAAACAGAGGATATCGGCATTTGTTTCTTTCTCTGAG
GACAAGAGAAAAAAGCCAGGTTCCACAGAGGACACAGAGAAGGTT
GACAAGAGAAAAAAGCCAGGTTCCACAGAGGACACAGAGAAGGTT
TGGGTGTCCTCCTGGGGTTCTTTTTGCCAACTTTCCCCACGTTAA
TGGGTGTCCTCCTGGGGTTCTTTTTGCCAACTTTCCCCACGTTAA
AGGTGAACATTGGTTCTTTCATTTGCTTTGGAAGTTTTAATCTCT
AGGTGAACATTGGTTCTTTCATTTGCTTTGGAAGTTTTAATCTCT
AACAGTGGACAAAGTTACCAGTGCCTTAAACTCTGTTACACTTTT
AACAGTGGACAAAGTTACCAGTGCCTTAAACTCTGTTACACTTTT
TGGAAGTGAAAACTTTGTAGTATGATAGGTTATTTTGATGTAAAG
TGGAAGTGAAAACTTTGTAGTATGATAGGTTATTTTGATGTAAAG
ATGTTCTGGATACCATTATATGTTCCCCCTGTTTCAGAGGCTCAG
ATGTTCTGGATACCATTATATGTTCCCCCTGTTTCAGAGGCTCAG
ATTGTAATATGTAAATGGTATGTCATTCGCTACTATGATTTAATT
ATTGTAATATGTAAATGGTATGTCATTCGCTACTATGATTTAATT
TGAAATATGGTCTTTTGGTTATGAATACTTTGCAGCACAGCTGAG
TGAAATATGGTCTTTTGGTTATGAATACTTTGCAGCACAGCTGAG
AGGCTGTCTGTTGTATTCATTGTGGTCATAGCACCTAACAACATT
AGGCTGTCTGTTGTATTCATTGTGGTCATAGCACCTAACAACATT
GTAGCCTCAATCGAGTGAGACAGACTAGAAGTTCCTAGTGATGGC
GTAGCCTCAATCGAGTGAGACAGACTAGAAGTTCCTAGTGATGGC
TTATGATAGCAAATGGCCTCATGTCAAATATTTAGATGTAATTTT
TTATGATAGCAAATGGCCTCATGTCAAATATTTAGATGTAATTTT
GTGTAAGAAATACAGACTGGATGTACCACCAACTACTACCTGTAA
GTGTAAGAAATACAGACTGGATGTACCACCAACTACTACCTGTAA
TGACAGGCCTGTCCAACACATCTCCCTTTTCCATGACTGTGGTAG
TGACAGGCCTGTCCAACACATCTCCCTTTTCCATGACTGTGGTAG
CCAGCATCGGAAAGAACGCTGATTTAAAGAGGTCGCTTGGGAATT
CCAGCATCGGAAAGAACGCTGATTTAAAGAGGTCGCTTGGGAATT
TTATTGACACAGTACCATTTAATGGGGAGGACAAAATGGGGCAGG
TTATTGACACAGTACCATTTAATGGGGAGGACAAAATGGGGCAGG
GGAGGGAGAAGTTTCTGTCGTTAAAAACAGATTTGGAAAGACTGG
GGAGGGAGAAGTTTCTGTCGTTAAAAACAGATTTGGAAAGACTGG
ACTCTAAAGTCTGTTGATTAAAGATGAGCTTTGTCTACTTCAAAA
ACTCTAAAGTCTGTTGATTAAAGATGAGCTTTGTCTACTTCAAAA
GTTTGTTTGCTTACCCCTTCAGCCTCCAATTTTTTAAGTGAAAAT
GTTTGTTTGCTTACCCCTTCAGCCTCCAATTTTTTAAGTGAAAAT
ATAGCTAATAACATGTGAAAAGAATAGAAGCTAAGGTTTAGATAA
ATAGCTAATAACATGTGAAAAGAATAGAAGCTAAGGTTTAGATAA
ATATTGAGCAGATCTATAGGAAGATTGAACCTGAATATTGCCATT
ATATTGAGCAGATCTATAGGAAGATTGAACCTGAATATTGCCATT
ATGCTTGACATGGTTTCCAAAAAATGGTACTCCACATATTTCAGT
ATGCTTGACATGGTTTCCAAAAAATGGTACTCCACATATTTCAGT
GAGGGTAAGTATTTTCCTGTTGTCAAGAATAGCATTGTAAAAGCA
GAGGGTAAGTATTTTCCTGTTGTCAAGAATAGCATTGTAAAAGCA
TTTTGTAATAATAAAGAATAGCTTTAATGATATGCTTGTAACTAA
TTTTGTAATAATAAAGAATAGCTTTAATGATATGCTTGTAACTAA
AATAATTTTGTAATGTATCAAATACATTTAAAACATTAAAATATA
AATAATTTTGTAATGTATCAAATACATTTAAAACATTAAAATATA
ATCTCTATAATAAGAGCTCGCTGATCAGCCTCGACTGTGCCTTCT
ATCTCTATAATAAGAGCTCGCTGATCAGCCTCGACTGTGCCTTCT
AGTTGCCAGCCATCTGTTGTTTGCCCCTCCCCCGTGCCTTCCTTG
AGTTGCCAGCCATCTGTTGTTTGCCCCTCCCCCGTGCCTTCCTTG
ACCCTGGAAGGTGCCACTCCCACTGTCCTTTCCTAATAAAATGAG
ACCCTGGAAGGTGCCACTCCCACTGTCCTTTCCTAATAAAATGAG
GAAATTGCATCGCATTGTCTGAGTAGGTGTCATTCTATTCTGGGG
GAAATTGCATCGCATTGTCTGAGTAGGTGTCATTCTATTCTGGGG
GGTGGGGTGGGGCAGGACAGCAAGGGGGAGGATTGGGAAGACAAT
GGTGGGGTGGGGCAGGACAGCAAGGGGGAGGATTGGGAAGACAAT
AGCAGGCATGCTGGGGATGCGGTGGGCTCTATGGAAGCTTGAATT
AGCAGGCATGCTGGGGATGCGGTGGGCTCTATGGAAGCTTGAATT
CAGCTGACGTGCCTCGGACCGCTAGGAACCCCTAGTGATGGAGTT
CAGCTGACGTGCCTCGGACCGCTAGGAACCCCTAGTGATGGAGTT
GGCCACTCCCTCTCTGCGCGCTCGCTCGCTCACTGAGGCCGGGCG
GGCCACTCCCTCTCTGCGCGCTCGCTCGCTCACTGAGGCCGGGCG
ACCAAAGGTCGCCCGACGCCCGGGCTTTGCCCGGGCGGCCTCAGT
ACCAAAGGTCGCCCGACGCCCGGGCTTTGCCCGGGCGGCCTCAGT
GAGCGAGCGAGCGCGCAGCTGCCTGCAGGGGCGCCTGATGCGGTA
GAGCGAGCGAGCGCGCAGCTGCCTGCAGGGGCGCCTGATGCGGTA
TTTTCTCCTTACGCATCTGTGCGGTATTTCACACCGCATACGTCA
TTTTCTCCTTACGCATCTGTGCGGTATTTCACACCGCATACGTCA
AAGCAACCATAGTACGCGCCCTGTAGCGGCGCATTAAGCGCGGCG
AAGCAACCATAGTACGCGCCCTGTAGCGGCGCATTAAGCGCGGCG
GGTGTGGTGGTTACGCGCAGCGTGACCGCTACACTTGCCAGCGCC
GGTGTGGTGGTTACGCGCAGCGTGACCGCTACACTTGCCAGCGCC
CTAGCGCCCGCTCCTTTCGCTTTCTTCCCTTCCTTTCTCGCCACG
CTAGCGCCCGCTCCTTTCGCTTTCTTCCCTTCCTTTCTCGCCACG
TTCGCCGGCTTTCCCCGTCAAGCTCTAAATCGGGGGCTCCCTTTA
TTCGCCGGCTTTCCCCGTCAAGCTCTAAATCGGGGGCTCCCTTTA
GGGTTCCGATTTAGTGCTTTACGGCACCTCGACCCCAAAAAACTT
GGGTTCCGATTTAGTGCTTTACGGCACCTCGACCCCAAAAAACTT
GATTTGGGTGATGGTTCACGTAGTGGGCCATCGCCCTGATAGACG
GATTTGGGTGATGGTTCACGTAGTGGGCCATCGCCCTGATAGACG
GTTTTTCGCCCTTTGACGTTGGAGTCCACGTTCTTTAATAGTGGA
GTTTTTCGCCCTTTGACGTTGGAGTCCACGTTCTTTAATAGTGGA
CTCTTGTTCCAAACTGGAACAACACTCAACCCTATCTCGGGCTAT
CTCTTGTTCCAAACTGGAACAACACTCAACCCTATCTCGGGCTAT
TCTTTTGATTTATAAGGGATTTTGCCGATTTCGGCCTATTGGTTA
TCTTTTGATTTATAAGGGATTTTGCCGATTTCGGCCTATTGGTTA
AAAAATGAGCTGATTTAACAAAAATTTAACGCGAATTTTAACAAA
AAAAATGAGCTGATTTAACAAAAATTTAACGCGAATTTTAACAAA
ATATTAACGTTTACAATTTTATGGTGCACTCTCAGTACAATCTGC
ATATTAACGTTTACAATTTTATGGTGCACTCTCAGTACAATCTGC
TCTGATGCCGCATAGTTAAGCCAGCCCCGACACCCGCCAACACCC
TCTGATGCCGCATAGTTAAGCCAGCCCCGACACCCGCCAACACCC
GCTGACGCGCCCTGACGGGCTTGTCTGCTCCCGGCATCCGCTTAC
GCTGACGCGCCCTGACGGGCTTGTCTGCTCCCGGCATCCGCTTAC
AGACAAGCTGTGACCGTCTCCGGGAGCTGCATGTGTCAGAGGTTT
AGACAAGCTGTGACCGTCTCCGGGAGCTGCATGTGTCAGAGGTTT
TCACCGTCATCACCGAAACGCGCGAGACGAAAGGGCCTCGTGATA
TCACCGTCATCACCGAAACGCGCGAGACGAAAGGGCCTCGTGATA
CGCCTATTTTTATAGGTTAATGTCATGAACAATAAAACTGTCTGC
CGCCTATTTTTATAGGTTAATGTCATGAACAATAAAACTGTCTGC
TTACATAAACAGTAATACAAGGGGTGTTATGAGCCATATTCAACG
TTACATAAACAGTAATACAAGGGGTGTTATGAGCCATATTCAACG
GGAAACGTCGAGGCCGCGATTAAATTCCAACATGGATGCTGATTT
GGAAACGTCGAGGCCGCGATTAAATTCCAACATGGATGCTGATTT
ATATGGGTATAAATGGGCTCGCGATAATGTCGGGCAATCAGGTGC
ATATGGGTATAAATGGGCTCGCGATAATGTCGGGCAATCAGGTGC
GACAATCTATCGCTTGTATGGGAAGCCCGATGCGCCAGAGTTGTT
GACAATCTATCGCTTGTATGGGAAGCCCGATGCGCCAGAGTTGTT
TCTGAAACATGGCAAAGGTAGCGTTGCCAATGATGTTACAGATGA
TCTGAAACATGGCAAAGGTAGCGTTGCCAATGATGTTACAGATGA
GATGGTCAGACTAAACTGGCTGACGGAATTTATGCCTCTTCCGAC
GATGGTCAGACTAAACTGGCTGACGGAATTTATGCCTCTTCCGAC
CATCAAGCATTTTATCCGTACTCCTGATGATGCATGGTTACTCAC
CATCAAGCATTTTATCCGTACTCCTGATGATGCATGGTTACTCAC
CACTGCGATCCCCGGAAAAACAGCATTCCAGGTATTAGAAGAATA
CACTGCGATCCCCGGAAAAACAGCATTCCAGGTATTAGAAGAATA
TCCTGATTCAGGTGAAAATATTGTTGATGCGCTGGCAGTGTTCCT
TCCTGATTCAGGTGAAAATATTGTTGATGCGCTGGCAGTGTTCCT
GCGCCGGTTGCATTCGATTCCTGTTTGTAATTGTCCTTTTAACAG
GCGCCGGTTGCATTCGATTCCTGTTTGTAATTGTCCTTTTAACAG
CGATCGCGTATTTCGTCTCGCTCAGGCGCAATCACGAATGAATAA
CGATCGCGTATTTCGTCTCGCTCAGGCGCAATCACGAATGAATAA
CGGTTTGGTTGATGCGAGTGATTTTGATGACGAGCGTAATGGCTG
CGGTTTGGTTGATGCGAGTGATTTTGATGACGAGCGTAATGGCTG
GCCTGTTGAACAAGTCTGGAAAGAAATGCATAAACTTTTGCCATT
GCCTGTTGAACAAGTCTGGAAAGAAATGCATAAACTTTTGCCATT
CTCACCGGATTCAGTCGTCACTCATGGTGATTTCTCACTTGATAA
CTCACCGGATTCAGTCGTCACTCATGGTGATTTCTCACTTGATAA
CCTTATTTTTGACGAGGGGAAATTAATAGGTTGTATTGATGTTGG
CCTTATTTTTGACGAGGGGAAATTAATAGGTTGTATTGATGTTGG
ACGAGTCGGAATCGCAGACCGATACCAGGATCTTGCCATCCTATG
ACGAGTCGGAATCGCAGACCGATACCAGGATCTTGCCATCCTATG
GAACTGCCTCGGTGAGTTTTCTCCTTCATTACAGAAACGGCTTTT
GAACTGCCTCGGTGAGTTTTCTCCTTCATTACAGAAACGGCTTTT
TCAAAAATATGGTATTGATAATCCTGATATGAATAAATTGCAGTT
TCAAAAATATGGTATTGATAATCCTGATATGAATAAATTGCAGTT
TCATTTGATGCTCGATGAGTTTTTCTAATCTCATGACCAAAATCC
TCATTTGATGCTCGATGAGTTTTTCTAATCTCATGACCAAAATCC
CTTAACGTGAGTTTTCGTTCCACTGAGCGTCAGACCCCGTAGAAA
CTTAACGTGAGTTTTCGTTCCACTGAGCGTCAGACCCCGTAGAAA
AGATCAAAGGATCTTCTTGAGATCCTTTTTTTCTGCGCGTAATCT
AGATCAAAGGATCTTCTTGAGATCCTTTTTTTCTGCGCGTAATCT
GCTGCTTGCAAACAAAAAAACCACCGCTACCAGCGGTGGTTTGTT
GCTGCTTGCAAACAAAAAAACCACCGCTACCAGCGGTGGTTTGTT
TGCCGGATCAAGAGCTACCAACTCTTTTTCCGAAGGTAACTGGCT
TGCCGGATCAAGAGCTACCAACTCTTTTTCCGAAGGTAACTGGCT
TCAGCAGAGCGCAGATACCAAATACTGTCCTTCTAGTGTAGCCGT
TCAGCAGAGCGCAGATACCAAATACTGTCCTTCTAGTGTAGCCGT
AGTTAGGCCACCACTTCAAGAACTCTGTAGCACCGCCTACATACC
AGTTAGGCCACCACTTCAAGAACTCTGTAGCACCGCCTACATACC
TCGCTCTGCTAATCCTGTTACCAGTGGCTGCTGCCAGTGGCGATA
TCGCTCTGCTAATCCTGTTACCAGTGGCTGCTGCCAGTGGCGATA
AGTCGTGTCTTACCGGGTTGGACTCAAGACGATAGTTACCGGATA
AGTCGTGTCTTACCGGGTTGGACTCAAGACGATAGTTACCGGATA
AGGCGCAGCGGTCGGGCTGAACGGGGGGTTCGTGCACACAGCCCA
AGGCGCAGCGGTCGGGCTGAACGGGGGGTTCGTGCACACAGCCCA
GCTTGGAGCGAACGACCTACACCGAACTGAGATACCTACAGCGTG
GCTTGGAGCGAACGACCTACACCGAACTGAGATACCTACAGCGTG
AGCTATGAGAAAGCGCCACGCTTCCCGAAGGGAGAAAGGCGGACA
AGCTATGAGAAAGCGCCACGCTTCCCGAAGGGAGAAAGGCGGACA
GGTATCCGGTAAGCGGCAGGGTCGGAACAGGAGAGCGCACGAGGG
GGTATCCGGTAAGCGGCAGGGTCGGAACAGGAGAGCGCACGAGGG
AGCTTCCAGGGGGAAACGCCTGGTATCTTTATAGTCCTGTCGGGT
AGCTTCCAGGGGGAAACGCCTGGTATCTTTATAGTCCTGTCGGGT
TTCGCCACCTCTGACTTGAGCGTCGATTTTTGTGATGCTCGTCAG
TTCGCCACCTCTGACTTGAGCGTCGATTTTTGTGATGCTCGTCAG
GGGGGCGGAGCCTATGGAAAAACGCCAGCAACGCGGCCTTTTTAC
GGGGGCGGAGCCTATGGAAAAACGCCAGCAACGCGGCCTTTTTAC
GGTTCCTGGCCTTTTGCTGGCCTTTTGCTCACATGT

TABLE 6
Components of Construct Sequence (SEQ ID NO: 17)

| Components | Position in construct |
|---|---|
| 5′ITR | 12-130 |
| Cloning site | 131-147 |
| RBP7 promoter | 148-1244 |
| hGJB2 minimal promoter | 1245-1372 |
| Cloning site | 1373-1381 |
| Synthetic barcode | 1382-1389 |
| 5′UTR | 1390-1751 |
| GJB2 (exon2) | 1763-2440 |
| 3xFLAG | 2453-2518 |
| 3′UTR (exon2) | 2522-3928 |
| bGHpA | 3950-4174 |
| Cloning site | 4175-4208 |
| 3′ITR | 4209-4338 |

Exemplary Construct sequence
(SEQ ID NO: 38)
CCTGCAGGCAGCTGCGCGCTCGCTCGCTCACTGAGGCCGCCCGGG
CGTCGGGCGACCTTTGGTCGCCCGGCCTCAGTGAGCGAGCGAGCG
CGCAGAGAGGGAGTGGCCAACTCCATCACTAGGGGTTCCTGCGGC
CGCACGCGTGGTAAATAGCTTCCAACGTTTCCACCCCACCAGCCC
TTGCACCACTCCCTGTACTGGCCCTGAGCTTTCTAGTCTTGACTG
AAAAGCGGGGAGGCAATGTGGTCTCTCCTGGTGCACTGTCCCGAG
GAAGGCCTGCTCCGCTTCCCCGGAGGAGTCTTCAAAGGATGGAGG
TAATTAATAAAAACAACCCCTGTACCTCCTCTAAGTGGTCATTAA
TTAATAAAGAACCTCCAGGCTCCTATAGGAGAGGTCTGTGCACCC
CGCGGGCTATGAGAAGGCTGGATCACCCAGAAAGACTGAGGATGT
GTCCTGGCAAAAACACAGCCTGCCCCTCACACTGCTCCCCACGGG
TGCACTAGGGAGGAAGAGTTCCCTCGAGGGCCTGAGCAGGCGCCC
CACACCTGCACCCGTGCAGAGGGGGCTGGGCCCGCCCTCTGCGCT
CCCGAGGGAGAGCCCTACCCCCTGCATCCCCGGTACCCCGTTCCC
TCCAAGGGCCGGAAAGAGGGCCCCGCGCACTGTGCACTTCTTAGG
GGTCCCCCACCCTGCGCCCCCGCCACGGGAAAAAGGTCCCCGCTC
TGCGCATCCGGCCCCGGAGGGACAGCCCCGGTCCTGCACTCCTTG
CTCCTCAGGGGGACGGTCCGCGCCCAGCGGCTAGTGCGCCCCGGG
TAGGTGGGGGCGGGGGGCTCGTCGAGTGACAGCGCTCGCCTCCCG
CAGCCCGCCCGAGCCGCGTCAGGGCAGAAGCTCTGAGGACCCAGA
GGCCGGGCGCGCTCCGCCCGCGGCGCCGCCCCCTCCGTAACTTTC
CCAGTCTCCGAGGGAAGAGGCGGGGTGTGGGGTGCGGTTAAAAGG
CGCCACGGCGGGAGACAGGTCTCACCGGTTCGTGGGTGTTGCGGC
CCCGCAGCGCCCGCGCGCTCCTCTCCCCGACTCGGAGCCCCTCGG
CGGCGCCCGGCCCAGGACCCGCCTAGGAGCGCAGGAGCCCCAGCG
CAGAGACCCCAACGCCGAGACCCCCGCCCCGGCCCCGCCGCGCTT
CCTCCCGACGCAGTTTAGGACCCTTGTTCGCGAAGAGGTGGTGTG
CGGCTGAGACCCGCGTCCTCAGGACGGTTCCATCAGTGCCTCGAT
CCTGCCCCACTGGAGGAGGAAGGCAGCCCGAACAGCGCTCACCTA
ACTAACAGCTGCTGAGAGCTGGGTTCCGTGGCCATGCACCTGGGA
CTGCCTTGAGAAGCGTGAGCAAACCGCCCAGAGTAGAAGCGCTAG
CCACCATGGATTGGGGCACGCTGCAGACGATCCTGGGGGGTGTGA
ACAAACACTCCACCAGCATTGGAAAGATCTGGCTCACCGTCCTCT
TCATTTTTCGCATTATGATCCTCGTTGTGGCTGCAAAGGAGGTGT
GGGGAGATGAGCAGGCCGACTTTGTCTGCAACACCCTGCAGCCAG
GCTGCAAGAACGTGTGCTACGATCACTACTTCCCCATCTCCCACA
TCCGGCTATGGGCCCTGCAGCTGATCTTCGTGTCCACGCCAGCGC
TCCTAGTGGCCATGCACGTGGCCTACCGGAGACATGAGAAGAAGA
GGAAGTTCATCAAGGGGGAGATAAAGAGTGAATTTAAGGACATCG
AGGAGATCAAAACCCAGAAGGTCCGCATCGAAGGCTCCCTGTGGT
GGACCTACACAAGCAGCATCTTCTTCCGGGTCATCTTCGAAGCCG
CCTTCATGTACGTCTTCTATGTCATGTACGACGGCTTCTCCATGC
AGCGGCTGGTGAAGTGCAACGCCTGGCCTTGTCCCAACACTGTGG
ACTGCTTTGTGTCCCGGCCCACGGAGAAGACTGTCTTCACAGTGT
TCATGATTGCAGTGTCTGGAATTTGCATCCTGCTGAATGTCACTG
AATTGTGTTATTTGCTAATTAGATATTGTTCTGGGAAGTCAAAAA
AGCCAGTTGGATCCCGGGCTGACTACAAAGACCATGACGGTGATT
ATAAAGATCATGACATCGACTACAAGGATGACGATGACAAGTAAG
AAATAGACAGCATGAGAGGGATGAGGCAACCCGTGCTCAGCTGTC
AAGGCTCAGTCGCTAGCATTTCCCAACACAAAGATTCTGACCTTA
AATGCAACCATTTGAAACCCCTGTAGGCCTCAGGTGAAACTCCAG
ATGCCACAATGGAGCTCTGCTCCCCTAAAGCCTCAAAACAAAGGC
CTAATTCTATGCCTGTCTTAATTTTCTTTCACTTAAGTTAGTTCC
ACTGAGACCCCAGGCTGTTAGGGGTTATTGGTGTAAGGTACTTTC
ATATTTTAAACAGAGGATATCGGCATTTGTTTCTTTCTCTGAGGA
CAAGAGAAAAAAGCCAGGTTCCACAGAGGACACAGAGAAGGTTTG
GGTGTCCTCCTGGGGTTCTTTTTGCCAACTTTCCCCACGTTAAAG
GTGAACATTGGTTCTTTCATTTGCTTTGGAAGTTTTAATCTCTAA
CAGTGGACAAAGTTACCAGTGCCTTAAACTCTGTTACACTTTTTG
GAAGTGAAAACTTTGTAGTATGATAGGTTATTTTGATGTAAAGAT
GTTCTGGATACCATTATATGTTCCCCCTGTTTCAGAGGCTCAGAT
TGTAATATGTAAATGGTATGTCATTCGCTACTATGATTTAATTTG
AAATATGGTCTTTTGGTTATGAATACTTTGCAGCACAGCTGAGAG
GCTGTCTGTTGTATTCATTGTGGTCATAGCACCTAACAACATTGT
AGCCTCAATCGAGTGAGACAGACTAGAAGTTCCTAGTGATGGCTT
ATGATAGCAAATGGCCTCATGTCAAATATTTAGATGTAATTTTGT
GTAAGAAATACAGACTGGATGTACCACCAACTACTACCTGTAATG
ACAGGCCTGTCCAACACATCTCCCTTTTCCATGACTGTGGTAGCC
AGCATCGGAAAGAACGCTGATTTAAAGAGGTCGCTTGGGAATTTT
ATTGACACAGTACCATTTAATGGGGAGGACAAAATGGGGCAGGGG
AGGGAGAAGTTTCTGTCGTTAAAAACAGATTTGGAAAGACTGGAC
TCTAAAGTCTGTTGATTAAAGATGAGCTTTGTCTACTTCAAAAGT
TTGTTTGCTTACCCCTTCAGCCTCCAATTTTTTAAGTGAAAATAT
AGCTAATAACATGTGAAAAGAATAGAAGCTAAGGTTTAGATAAAT
ATTGAGCAGATCTATAGGAAGATTGAACCTGAATATTGCCATTAT
GCTTGACATGGTTTCCAAAAAATGGTACTCCACATATTTCAGTGA
GGGTAAGTATTTTCCTGTTGTCAAGAATAGCATTGTAAAAGCATT
TTGTAATAATAAAGAATAGCTTTAATGATATGCTTGTAACTAAAA
TAATTTTGTAATGTATCAAATACATTTAAAACATTAAAATATAAT
CTCTATAATAAGAGCTCGCTGATCAGCCTCGACTGTGCCTTCTAG
TTGCCAGCCATCTGTTGTTTGCCCCTCCCCCGTGCCTTCCTTGAC
CCTGGAAGGTGCCACTCCCACTGTCCTTTCCTAATAAAATGAGGA
AATTGCATCGCATTGTCTGAGTAGGTGTCATTCTATTCTGGGGGG
GGGGTGGGGCAGGACAGCAAGGGGGAGGATTGGGAAGACAATAGC
AGGCATGCTGGGGATGCGGTGGGCTCTATGGAAGCTTGAATTCAG
CTGACGTGCCTCGGACCGCTAGGAACCCCTAGTGATGGAGTTGGC
CACTCCCTCTCTGCGCGCTCGCTCGCTCACTGAGGCCGGGCGACC
AAAGGTCGCCCGACGCCCGGGCTTTGCCCGGGCGGCCTCAGTGAG
CGAGCGAGCGCGCAGCTGCCTGCAGGGGCGCCTGATGCGGTATTT
TCTCCTTACGCATCTGTGCGGTATTTCACACCGCATACGTCAAAG
CAACCATAGTACGCGCCCTGTAGCGGCGCATTAAGCGCGGCGGGT
GTGGTGGTTACGCGCAGCGTGACCGCTACACTTGCCAGCGCCCTA
GCGCCCGCTCCTTTCGCTTTCTTCCCTTCCTTTCTCGCCACGTTC
GCCGGCTTTCCCCGTCAAGCTCTAAATCGGGGGCTCCCTTTAGGG
TTCCGATTTAGTGCTTTACGGCACCTCGACCCCAAAAAACTTGAT
TTGGGTGATGGTTCACGTAGTGGGCCATCGCCCTGATAGACGGTT
TTTCGCCCTTTGACGTTGGAGTCCACGTTCTTTAATAGTGGACTC
TTGTTCCAAACTGGAACAACACTCAACCCTATCTCGGGCTATTCT
TTTGATTTATAAGGGATTTTGCCGATTTCGGCCTATTGGTTAAAA
AATGAGCTGATTTAACAAAAATTTAACGCGAATTTTAACAAAATA
TTAACGTTTACAATTTTATGGTGCACTCTCAGTACAATCTGCTCT
GATGCCGCATAGTTAAGCCAGCCCCGACACCCGCCAACACCCGCT
GACGCGCCCTGACGGGCTTGTCTGCTCCCGGCATCCGCTTACAGA
CAAGCTGTGACCGTCTCCGGGAGCTGCATGTGTCAGAGGTTTTCA
CCGTCATCACCGAAACGCGCGAGACGAAAGGGCCTCGTGATACGC
CTATTTTTATAGGTTAATGTCATGAACAATAAAACTGTCTGCTTA
CATAAACAGTAATACAAGGGGTGTTATGAGCCATATTCAACGGGA
AACGTCGAGGCCGCGATTAAATTCCAACATGGATGCTGATTTATA
TGGGTATAAATGGGCTCGCGATAATGTCGGGCAATCAGGTGCGAC
AATCTATCGCTTGTATGGGAAGCCCGATGCGCCAGAGTTGTTTCT
GAAACATGGCAAAGGTAGCGTTGCCAATGATGTTACAGATGAGAT
GGTCAGACTAAACTGGCTGACGGAATTTATGCCTCTTCCGACCAT
CAAGCATTTTATCCGTACTCCTGATGATGCATGGTTACTCACCAC
TGCGATCCCCGGAAAAACAGCATTCCAGGTATTAGAAGAATATCC
TGATTCAGGTGAAAATATTGTTGATGCGCTGGCAGTGTTCCTGCG
CCGGTTGCATTCGATTCCTGTTTGTAATTGTCCTTTTAACAGCGA
TCGCGTATTTCGTCTCGCTCAGGCGCAATCACGAATGAATAACGG
TTTGGTTGATGCGAGTGATTTTGATGACGAGCGTAATGGCTGGCC
TGTTGAACAAGTCTGGAAAGAAATGCATAAACTTTTGCCATTCTC
ACCGGATTCAGTCGTCACTCATGGTGATTTCTCACTTGATAACCT
TATTTTTGACGAGGGGAAATTAATAGGTTGTATTGATGTTGGACG
AGTCGGAATCGCAGACCGATACCAGGATCTTGCCATCCTATGGAA
CTGCCTCGGTGAGTTTTCTCCTTCATTACAGAAACGGCTTTTTCA
AAAATATGGTATTGATAATCCTGATATGAATAAATTGCAGTTTCA
TTTGATGCTCGATGAGTTTTTCTAATCTCATGACCAAAATCCCTT
AACGTGAGTTTTCGTTCCACTGAGCGTCAGACCCCGTAGAAAAGA
TCAAAGGATCTTCTTGAGATCCTTTTTTTCTGCGCGTAATCTGCT
GCTTGCAAACAAAAAAACCACCGCTACCAGCGGTGGTTTGTTTGC
CGGATCAAGAGCTACCAACTCTTTTTCCGAAGGTAACTGGCTTCA
GCAGAGCGCAGATACCAAATACTGTCCTTCTAGTGTAGCCGTAGT
TAGGCCACCACTTCAAGAACTCTGTAGCACCGCCTACATACCTCG
CTCTGCTAATCCTGTTACCAGTGGCTGCTGCCAGTGGCGATAAGT
CGTGTCTTACCGGGTTGGACTCAAGACGATAGTTACCGGATAAGG
CGCAGCGGTCGGGCTGAACGGGGGGTTCGTGCACACAGCCCAGCT
TGGAGCGAACGACCTACACCGAACTGAGATACCTACAGCGTGAGC
TATGAGAAAGCGCCACGCTTCCCGAAGGGAGAAAGGCGGACAGGT
ATCCGGTAAGCGGCAGGGTCGGAACAGGAGAGCGCACGAGGGAGC
TTCCAGGGGGAAACGCCTGGTATCTTTATAGTCCTGTCGGGTTTC
GCCACCTCTGACTTGAGCGTCGATTTTTGTGATGCTCGTCAGGGG
GGCGGAGCCTATGGAAAAACGCCAGCAACGCGGCCTTTTTACGGT
TCCTGGCCTTTTGCTGGCCTTTTGCTCACATGT

TABLE 7
Components of Construct Sequence (SEQ ID NO: 38)

| Components | Position in construct |
|---|---|
| 5′ITR | 12-130 |
| Cloning site | 131-147 |
| GJB6 promoter | 148-882 |
| hGJB2 minimal promoter | 883-1010 |
| Cloning site | 1011-1019 |
| Synthetic barcode | 1020-1027 |
| 5′UTR | 1028-1389 |
| GJB2 (exon2) | 1401-2078 |
| 3xFLAG | 2091-2156 |
| 3′UTR (exon2) | 2160-3566 |
| bGHpA | 3588-3812 |
| Cloning site | 3813-3846 |
| 3′ITR | 3847-3976 |

Exemplary Construct sequence
(SEQ ID NO: 7)
CCTGCAGGCAGCTGCGCGCTCGCTCGCTCACTGAGGCCGCCCGGG
CGTCGGGCGACCTTTGGTCGCCCGGCCTCAGTGAGCGAGCGAGCG
CGCAGAGAGGGAGTGGCCAACTCCATCACTAGGGGTTCCTGCGGC
CGCACGCGTGGTTGTACAGGAGATAGTCAGGGAATTAGTAATTTT
CAAAGAGGTGACTTTGAATTCAAACTTAAATATCATCTTCAGCTG
AAACAAAGAAGGGGTGCAGTTATGAGGAAGTGACCAGGTAAAGCA
TGGCAAACAAAGGTAAAGTTTGTTATGCGTATTTAAGTCAGAGCC
CTCTCCATTGATAAGAGTTTCCAGTAATTTAGTGCCATCCTTTTC
TTGCTATAGAGTTCTCGTCTCTATCTGAGCACGCAAAAATAACAT
GCTTTCTTGCTTTCTTGAAGTTGGGCATGGCCATTGACTTGCCTT
AGCCCATATTTTTCTGTGAAGTGGTCTTCAAAAACCTATATTTCT
GCCATAGAGTCACTTACTTAACCTGCCCTATTTAAAGGGGCTAAT
GCCTGATAGAATGTCGCTGCATAACTCCATCTGTGTGTGGTCCCT
GCATCCATGACAACCAAAACCCAGATGCAGAAATTGTTCCTAATC
ACATAGATTACCCTAGAAACCGGAAGGGCCTTGAAGTCAAAAGCA
TTCAGAGAACATGCTGAACAAATTGAATTTGCAGTTTATCTGGCC
AGGGAGGATGGAGAGGGGATGGGCACTTGGTCTGAGTATTTTTTG
TTTCTCATTCCAACAGAAATTACTAGATTTACCAAAAAATCTACA
AGTGGTAGTGTGATAGAGTCAGGCAGAGGAATTGACCATAGATAA
GGTGCTCAGGACTCCTAGAGTCAGCTTCTGGTATGTGAGAAAGAA
GTGAGAACAGAGCCCATGGCATATGAAGAAGATATTACAGAAAAA
AGAAAGCTGCCTTCCACGCAAATCATTTCTTTACAAAGGCTTGTT
AACTCCTGCAGTGCCAAGAAGCTGAATGCAGCGGCAGACATCCTG
GTTCGGGCCCCAGGAAGCTCAGCCGGGTTTAATGTGGATGAGGGT
TTAATGATGTACACGCAGAAGTGTTTTGACAAATGAAGAAGGTCC
TCATTCTTGGAACATGTGCCGGTTCTCCGAGGGAACTCCTAAAAG
GCTGTAAGCTCATGTAGGAAAAGCTGAGCTAGATTCCTAAGGGCA
GAGATGTGCTCACATTTCTTTGCATCCCTAGTTCCCAGCACAGTG
CAAGGCGCTGCAAACATTTGCTGAACCCAGGGTCTCGTGTCTT7G
ACTGTCCAGCAGAGGCCGCTCTGGGCCGGGGCTCTCGGGACCTGA
GGGCTGAGAGAAGGAAGGCCAGGGGGTGGCCCAGTCATCGCCGCG
GGGCCCGGGTGGGAGGGGTTTGGCAGCGGCAGGCGCGGCGGCGGC
GGCGGAGGCGGAGGCGGCCCCGGGAAGCTCTGAGGACCCAGAGGC
CGGGCGCGCTCCGCCCGCGGCGCCGCCCCCTCCGTAACTTTCCCA
GTCTCCGAGGGAAGAGGCGGGGTGTGGGGTGCGGTTAAAAGGCGC
CACGGCGGGAGACAGGTCTCACCGGTGCAAACTGGTTGCGGCCCC
GCAGCGCCCGCGCGCTCCTCTCCCCGACTCGGAGCCCCTCGGCGG
CGCCCGGCCCAGGACCCGCCTAGGAGCGCAGGAGCCCCAGCGCAG
AGACCCCAACGCCGAGACCCCCGCCCCGGCCCCGCCGCGCTTCCT
CCCGACGCAGTTTAGGACCCTTGTTCGCGAAGAGGTGGTGTGCGG
CTGAGACCCGCGTCCTCAGGACGGTTCCATCAGTGCCTCGATCCT
GCCCCACTGGAGGAGGAAGGCAGCCCGAACAGCGCTCACCTAACT
AACAGCTGCTGAGAGCTGGGTTCCGTGGCCATGCACCTGGGACTG
CCTTGAGAAGCGTGAGCAAACCGCCCAGAGTAGAAGCGCTAGCCA
CCATGGATTGGGGCACGCTGCAGACGATCCTGGGGGGTGTGAACA
AACACTCCACCAGCATTGGAAAGATCTGGCTCACCGTCCTCTTCA
TTTTTCGCATTATGATCCTCGTTGTGGCTGCAAAGGAGGTGTGGG
GAGATGAGCAGGCCGACTTTGTCTGCAACACCCTGCAGCCAGGCT
GCAAGAACGTGTGCTACGATCACTACTTCCCCATCTCCCACATCC
GGCTATGGGCCCTGCAGCTGATCTTCGTGTCCACGCCAGCGCTCC
TAGTGGCCATGCACGTGGCCTACCGGAGACATGAGAAGAAGAGGA
AGTTCATCAAGGGGGAGATAAAGAGTGAATTTAAGGACATCGAGG
AGATCAAAACCCAGAAGGTCCGCATCGAAGGCTCCCTGTGGTGGA
CCTACACAAGCAGCATCTTCTTCCGGGTCATCTTCGAAGCCGCCT
TCATGTACGTCTTCTATGTCATGTACGACGGCTTCTCCATGCAGC
GGCTGGTGAAGTGCAACGCCTGGCCTTGTCCCAACACTGTGGACT
GCTTTGTGTCCCGGCCCACGGAGAAGACTGTCTTCACAGTGTTCA
TGATTGCAGTGTCTGGAATTTGCATCCTGCTGAATGTCACTGAAT
TGTGTTATTTGCTAATTAGATATTGTTCTGGGAAGTCAAAAAAGC
CAGTTGGATCCCGGGCTGACTACAAAGACCATGACGGTGATTATA
AAGATCATGACATCGACTACAAGGATGACGATGACAAGTAAGAAA
TAGACAGCATGAGAGGGATGAGGCAACCCGTGCTCAGCTGTCAAG
GCTCAGTCGCTAGCATTTCCCAACACAAAGATTCTGACCTTAAAT
GCAACCATTTGAAACCCCTGTAGGCCTCAGGTGAAACTCCAGATG
CCACAATGGAGCTCTGCTCCCCTAAAGCCTCAAAACAAAGGCCTA
ATTCTATGCCTGTCTTAATTTTCTTTCACTTAAGTTAGTTCCACT
GAGACCCCAGGCTGTTAGGGGTTATTGGTGTAAGGTACTTTCATA
TTTTAAACAGAGGATATCGGCATTTGTTTCTTTCTCTGAGGACAA
GAGAAAAAAGCCAGGTTCCACAGAGGACACAGAGAAGGTTTGGGT
GTCCTCCTGGGGTTCTTTTTGCCAACTTTCCCCACGTTAAAGGTG
AACATTGGTTCTTTCATTTGCTTTGGAAGTTTTAATCTCTAACAG
TGGACAAAGTTACCAGTGCCTTAAACTCTGTTACACTTTTTGGAA
GTGAAAACTTTGTAGTATGATAGGTTATTTTGATGTAAAGATGTT
CTGGATACCATTATATGTTCCCCCTGTTTCAGAGGCTCAGATTGT
AATATGTAAATGGTATGTCATTCGCTACTATGATTTAATTTGAAA
TATGGTCTTTTGGTTATGAATACTTTGCAGCACAGCTGAGAGGCT
GTCTGTTGTATTCATTGTGGTCATAGCACCTAACAACATTGTAGC
CTCAATCGAGTGAGACAGACTAGAAGTTCCTAGTGATGGCTTATG
ATAGCAAATGGCCTCATGTCAAATATTTAGATGTAATTTTGTGTA
AGAAATACAGACTGGATGTACCACCAACTACTACCTGTAATGACA
GGCCTGTCCAACACATCTCCCTTTTCCATGACTGTGGTAGCCAGC
ATCGGAAAGAACGCTGATTTAAAGAGGTCGCTTGGGAATTTTATT
GACACAGTACCATTTAATGGGGAGGACAAAATGGGGCAGGGGAGG
GAGAAGTTTCTGTCGTTAAAAACAGATTTGGAAAGACTGGACTCT
AAAGTCTGTTGATTAAAGATGAGCTTTGTCTACTTCAAAAGTTTG
TTTGCTTACCCCTTCAGCCTCCAATTTTTTAAGTGAAAATATAGC
TAATAACATGTGAAAAGAATAGAAGCTAAGGTTTAGATAAATATT
GAGCAGATCTATAGGAAGATTGAACCTGAATATTGCCATTATGCT
TGACATGGTTTCCAAAAAATGGTACTCCACATATTTCAGTGAGGG
TAAGTATTTTCCTGTTGTCAAGAATAGCATTGTAAAAGCATTTTG
TAATAATAAAGAATAGCTTTAATGATATGCTTGTAACTAAAATAA
TTTTGTAATGTATCAAATACATTTAAAACATTAAAATATAATCTC
TATAATAAGAGCTCGCTGATCAGCCTCGACTGTGCCTTCTAGTTG
CCAGCCATCTGTTGTTTGCCCCTCCCCCGTGCCTTCCTTGACCCT
GGAAGGTGCCACTCCCACTGTCCTTTCCTAATAAAATGAGGAAAT
TGCATCGCATTGTCTGAGTAGGTGTCATTCTATTCTGGGGGGGGG
GTGGGGCAGGACAGCAAGGGGGAGGATTGGGAAGACAATAGCAGG
CATGCTGGGGATGCGGTGGGCTCTATGGAAGCTTGAATTCAGCTG
ACGTGCCTCGGACCGCTAGGAACCCCTAGTGATGGAGTTGGCCAC
TCCCTCTCTGCGCGCTCGCTCGCTCACTGAGGCCGGGCGACCAAA
GGTCGCCCGACGCCCGGGCTTTGCCCGGGCGGCCTCAGTGAGCGA
GCGAGCGCGCAGCTGCCTGCAGGGGCGCCTGATGCGGTATTTTCT
CCTTACGCATCTGTGCGGTATTTCACACCGCATACGTCAAAGCAA
CCATAGTACGCGCCCTGTAGCGGCGCATTAAGCGCGGCGGGTGTG
GTGGTTACGCGCAGCGTGACCGCTACACTTGCCAGCGCCCTAGCG
CCCGCTCCTTTCGCTTTCTTCCCTTCCTTTCTCGCCACGTTCGCC
GGCTTTCCCCGTCAAGCTCTAAATCGGGGGCTCCCTTTAGGGTTC
CGATTTAGTGCTTTACGGCACCTCGACCCCAAAAAACTTGATTTG
GGTGATGGTTCACGTAGTGGGCCATCGCCCTGATAGACGGTTTTT
CGCCCTTTGACGTTGGAGTCCACGTTCTTTAATAGTGGACTCTTG
TTCCAAACTGGAACAACACTCAACCCTATCTCGGGCTATTCTTTT
GATTTATAAGGGATTTTGCCGATTTCGGCCTATTGGTTAAAAAAT
GAGCTGATTTAACAAAAATTTAACGCGAATTTTAACAAAATATTA
ACGTTTACAATTTTATGGTGCACTCTCAGTACAATCTGCTCTGAT
GCCGCATAGTTAAGCCAGCCCCGACACCCGCCAACACCCGCTGAC
GCGCCCTGACGGGCTTGTCTGCTCCCGGCATCCGCTTACAGACAA
GCTGTGACCGTCTCCGGGAGCTGCATGTGTCAGAGGTTTTCACCG
TCATCACCGAAACGCGCGAGACGAAAGGGCCTCGTGATACGCCTA
TTTTTATAGGTTAATGTCATGAACAATAAAACTGTCTGCTTACAT
AAACAGTAATACAAGGGGTGTTATGAGCCATATTCAACGGGAAAC
GTCGAGGCCGCGATTAAATTCCAACATGGATGCTGATTTATATGG
GTATAAATGGGCTCGCGATAATGTCGGGCAATCAGGTGCGACAAT
CTATCGCTTGTATGGGAAGCCCGATGCGCCAGAGTTGTTTCTGAA
ACATGGCAAAGGTAGCGTTGCCAATGATGTTACAGATGAGATGGT
CAGACTAAACTGGCTGACGGAATTTATGCCTCTTCCGACCATCAA
GCATTTTATCCGTACTCCTGATGATGCATGGTTACTCACCACTGC
GATCCCCGGAAAAACAGCATTCCAGGTATTAGAAGAATATCCTGA
TTCAGGTGAAAATATTGTTGATGCGCTGGCAGTGTTCCTGCGCCG
GTTGCATTCGATTCCTGTTTGTAATTGTCCTTTTAACAGCGATCG
CGTATTTCGTCTCGCTCAGGCGCAATCACGAATGAATAACGGTTT
GGTTGATGCGAGTGATTTTGATGACGAGCGTAATGGCTGGCCTGT
TGAACAAGTCTGGAAAGAAATGCATAAACTTTTGCCATTCTCACC
GGATTCAGTCGTCACTCATGGTGATTTCTCACTTGATAACCTTAT
TTTTGACGAGGGGAAATTAATAGGTTGTATTGATGTTGGACGAGT
CGGAATCGCAGACCGATACCAGGATCTTGCCATCCTATGGAACTG
CCTCGGTGAGTTTTCTCCTTCATTACAGAAACGGCTTTTTCAAAA
ATATGGTATTGATAATCCTGATATGAATAAATTGCAGTTTCATTT
GATGCTCGATGAGTTTTTCTAATCTCATGACCAAAATCCCTTAAC
GTGAGTTTTCGTTCCACTGAGCGTCAGACCCCGTAGAAAAGATCA
AAGGATCTTCTTGAGATCCTTTTTTTCTGCGCGTAATCTGCTGCT
TGCAAACAAAAAAACCACCGCTACCAGCGGTGGTTTGTTTGCCGG
ATCAAGAGCTACCAACTCTTTTTCCGAAGGTAACTGGCTTCAGCA
GAGCGCAGATACCAAATACTGTCCTTCTAGTGTAGCCGTAGTTAG
GCCACCACTTCAAGAACTCTGTAGCACCGCCTACATACCTCGCTC
TGCTAATCCTGTTACCAGTGGCTGCTGCCAGTGGCGATAAGTCGT
GTCTTACCGGGTTGGACTCAAGACGATAGTTACCGGATAAGGCGC
AGCGGTCGGGCTGAACGGGGGGTTCGTGCACACAGCCCAGCTTGG
AGCGAACGACCTACACCGAACTGAGATACCTACAGCGTGAGCTAT
GAGAAAGCGCCACGCTTCCCGAAGGGAGAAAGGCGGACAGGTATC
CGGTAAGCGGCAGGGTCGGAACAGGAGAGCGCACGAGGGAGCTTC
CAGGGGGAAACGCCTGGTATCTTTATAGTCCTGTCGGGTTTCGCC
ACCTCTGACTTGAGCGTCGATTTTTGTGATGCTCGTCAGGGGGGC
GGAGCCTATGGAAAAACGCCAGCAACGCGGCCTTTTTACGGTTCC
TGGCCTTTTGCTGGCCTTTTGCTCACATGT

TABLE 8
Components of Construct Sequence (SEQ ID NO: 7)

| Components | Position in construct |
|---|---|
| 5′ITR | 12-130 |
| Cloning site | 131-147 |
| PARM1 promoter | 148-1463 |
| hGJB2 minimal promoter | 1464-1591 |
| Cloning site | 1592-1600 |
| Synthetic barcode | 1601-1608 |
| 5′UTR | 1609-1970 |
| GJB2 (exon2) | 1982-2659 |
| 3xFLAG | 2672-2737 |
| 3′UTR (exon2) | 2741-4147 |
| bGHpA | 4169-4393 |
| Cloning site | 4394-4427 |
| 3′ITR | 4428-4557 |

Exemplary Construct sequence
(SEQ ID NO: 100)
CCTGCAGGCAGCTGCGCGCTCGCTCGCTCACTGAGGCCGCCCGGG
CGTCGGGCGACCTTTGGTCGCCCGGCCTCAGTGAGCGAGCGAGCG
CGCAGAGAGGGAGTGGCCAACTCCATCACTAGGGGTTCCTGCGGC
CGCACGCGTGGTTGTGCTGCGAGGGCTTCATCTCCTAAGCACTAA
ATGCTAAATTCCCCCTCCCACGCCCATCGCCACTGTCCTCACGGA
TCCTCGCAGCAGCTTCCCAATCGGTCTCCCTGTCTCCAGCCTCAC
CACCCCCAACTAAGACCATTCATGAAAACAGAGACAACCAAGGAG
ACAGTCACCCAATGCTGTCCCTTCAGCTTGCATTATTTTCTGACA
AGACAGCTCTGCCATCCATGGAAGCCTGTGTTTGAAGATCTCTGA
CATAAAGGTCCCTTGCAGAGCTAGACGTGATTCTAAAATTGGGAA
CACAGGAATAAAAATCAAATCTTGAGTAGAAGTAGCTGAAAATTG
CAGTGATTCGGGGAAGCTTGGCTTCTAACTCCCCACTGTTTGAAG
ATGGGCTTGTTTGTTTTTTAAAACAGCCAACATAATTCAGCTGGA
GGAGGTACAAAGAATTTTCTATTCCTTGTTTCTGTAGAAATCGAT
GGACTTTAGCTTGTCTAATTGTCCCCCCTGCCTTTAGTATCTAAA
ATAAAATAACCCTCGTTGCTTGCATTACTCAACGCATTTCTGCGT
CTTGGCGTCTATGGCTAAACGAGTATTAATTAGACAGTCCGCAGA
GAGCTGGCTGGGGATAGAAGGGGAGGTGGGGGAGAAGGGCAGGGA
TCACAGCAGGGTGGACTCGTGGCCCTGATTTGGGATCCTGACAGC
AACTTACTAGGTGGCCTGAGGGCTGGGTGCCAGGGGAGGCAGCGG
GTTCCAGTAGCATCTGACCTGCATTAGGGACAGGGGCGCGGCGGA
GGGGGCGAAGGGGGCGGGGGTGGGGGGAAGGTGGCTGGGGTGAAG
CCCAGCTTCGCAGCTAGCTGTGGGCAACAGAGGGAGTAAGGGGGG
GCAATGAGGCTGGGGCCAGGCGCCAGCAGCAGCCACGCCCCCCAC
CTCCCCCGATTTTTAGGGAAAATTCTCCAAAGCTCTCGCATCCTC
CTCTGCCTCCTTCCACCCTCCACCCTCCCAGCCTCCACTGAGACC
TCTTTAAAACCACCCAGGGGCCGCCGGGGGATGAGGCCGGGGAAC
GGGCTGGACTGAGGGCGGGGGCTCGGGGGCAGCGGACGGGAAACG
CCTCGAAAGCAGCCAGACCCGGCGACTGAAATGAGGCGGAGGAGC
TTGGCGAGGGGAGGCGCAGGCTCGGAAAGGCGCGCGAGGCTCCAG
GCTCCTTCCCGATCCACCGCTCTCCTCGCTGACCTCCGAGTCACC
CCCGGAAGCTCCCGCCACTGCCGGGCGAATAGACCCCCGCGGACC
CCCAAGCGCGCGGGGCCGGGGCCCTAGTTCAGGCCCTCGCTGCCC
CTTTAAGGGTTCTCGAAACTTTCCCCCCGGTATCAGATGAGCCTC
GTCACATCCGTTGGCCGTGGCAAGCTCTGAGGACCCAGAGGCCGG
GCGCGCTCCGCCCGCGGCGCCGCCCCCTCCGTAACTTTCCCAGTC
TCCGAGGGAAGAGGCGGGGTGTGGGGTGCGGTTAAAAGGCGCCAC
GGCGGGAGACAGGTCTCACCGGTCCTACGCTGTTGCGGCCCCGCA
GCGCCCGCGCGCTCCTCTCCCCGACTCGGAGCCCCTCGGCGGCGC
CCGGCCCAGGACCCGCCTAGGAGCGCAGGAGCCCCAGCGCAGAGA
CCCCAACGCCGAGACCCCCGCCCCGGCCCCGCCGCGCTTCCTCCC
GACGCAGTTTAGGACCCTTGTTCGCGAAGAGGTGGTGTGCGGCTG
AGACCCGCGTCCTCAGGACGGTTCCATCAGTGCCTCGATCCTGCC
CCACTGGAGGAGGAAGGCAGCCCGAACAGCGCTCACCTAACTAAC
AGCTGCTGAGAGCTGGGTTCCGTGGCCATGCACCTGGGACTGCCT
TGAGAAGCGTGAGCAAACCGCCCAGAGTAGAAGCGCTAGCCACCA
TGGATTGGGGCACGCTGCAGACGATCCTGGGGGGTGTGAACAAAC
ACTCCACCAGCATTGGAAAGATCTGGCTCACCGTCCTCTTCATTT
TTCGCATTATGATCCTCGTTGTGGCTGCAAAGGAGGTGTGGGGAG
ATGAGCAGGCCGACTTTGTCTGCAACACCCTGCAGCCAGGCTGCA
AGAACGTGTGCTACGATCACTACTTCCCCATCTCCCACATCCGGC
TATGGGCCCTGCAGCTGATCTTCGTGTCCACGCCAGCGCTCCTAG
TGGCCATGCACGTGGCCTACCGGAGACATGAGAAGAAGAGGAAGT
TCATCAAGGGGGAGATAAAGAGTGAATTTAAGGACATCGAGGAGA
TCAAAACCCAGAAGGTCCGCATCGAAGGCTCCCTGTGGTGGACCT
ACACAAGCAGCATCTTCTTCCGGGTCATCTTCGAAGCCGCCTTCA
TGTACGTCTTCTATGTCATGTACGACGGCTTCTCCATGCAGCGGC
TGGTGAAGTGCAACGCCTGGCCTTGTCCCAACACTGTGGACTGCT
TTGTGTCCCGGCCCACGGAGAAGACTGTCTTCACAGTGTTCATGA
TTGCAGTGTCTGGAATTTGCATCCTGCTGAATGTCACTGAATTGT
GTTATTTGCTAATTAGATATTGTTCTGGGAAGTCAAAAAAGCCAG
TTGGATCCCGGGCTGACTACAAAGACCATGACGGTGATTATAAAG
ATCATGACATCGACTACAAGGATGACGATGACAAGTAAGAAATAG
ACAGCATGAGAGGGATGAGGCAACCCGTGCTCAGCTGTCAAGGCT
CAGTCGCTAGCATTTCCCAACACAAAGATTCTGACCTTAAATGCA
ACCATTTGAAACCCCTGTAGGCCTCAGGTGAAACTCCAGATGCCA
CAATGGAGCTCTGCTCCCCTAAAGCCTCAAAACAAAGGCCTAATT
CTATGCCTGTCTTAATTTTCTTTCACTTAAGTTAGTTCCACTGAG
ACCCCAGGCTGTTAGGGGTTATTGGTGTAAGGTACTTTCATATTT
TAAACAGAGGATATCGGCATTTGTTTCTTTCTCTGAGGACAAGAG
AAAAAAGCCAGGTTCCACAGAGGACACAGAGAAGGTTTGGGTGTC
CTCCTGGGGTTCTTTTTGCCAACTTTCCCCACGTTAAAGGTGAAC
ATTGGTTCTTTCATTTGCTTTGGAAGTTTTAATCTCTAACAGTGG
ACAAAGTTACCAGTGCCTTAAACTCTGTTACACTTTTTGGAAGTG
AAAACTTTGTAGTATGATAGGTTATTTTGATGTAAAGATGTTCTG
GATACCATTATATGTTCCCCCTGTTTCAGAGGCTCAGATTGTAAT
ATGTAAATGGTATGTCATTCGCTACTATGATTTAATTTGAAATAT
GGTCTTTTGGTTATGAATACTTTGCAGCACAGCTGAGAGGCTGTC
TGTTGTATTCATTGTGGTCATAGCACCTAACAACATTGTAGCCTC
AATCGAGTGAGACAGACTAGAAGTTCCTAGTGATGGCTTATGATA
GCAAATGGCCTCATGTCAAATATTTAGATGTAATTTTGTGTAAGA
AATACAGACTGGATGTACCACCAACTACTACCTGTAATGACAGGC
CTGTCCAACACATCTCCCTTTTCCATGACTGTGGTAGCCAGCATC
GGAAAGAACGCTGATTTAAAGAGGTCGCTTGGGAATTTTATTGAC
ACAGTACCATTTAATGGGGAGGACAAAATGGGGCAGGGGAGGGAG
AAGTTTCTGTCGTTAAAAACAGATTTGGAAAGACTGGACTCTAAA
GTCTGTTGATTAAAGATGAGCTTTGTCTACTTCAAAAGTTTGTTT
GCTTACCCCTTCAGCCTCCAATTTTTTAAGTGAAAATATAGCTAA
TAACATGTGAAAAGAATAGAAGCTAAGGTTTAGATAAATATTGAG
CAGATCTATAGGAAGATTGAACCTGAATATTGCCATTATGCTTGA
CATGGTTTCCAAAAAATGGTACTCCACATATTTCAGTGAGGGTAA
GTATTTTCCTGTTGTCAAGAATAGCATTGTAAAAGCATTTTGTAA
TAATAAAGAATAGCTTTAATGATATGCTTGTAACTAAAATAATTT
TGTAATGTATCAAATACATTTAAAACATTAAAATATAATCTCTAT
AATAAGAGCTCGCTGATCAGCCTCGACTGTGCCTTCTAGTTGCCA
GCCATCTGTTGTTTGCCCCTCCCCCGTGCCTTCCTTGACCCTGGA
AGGTGCCACTCCCACTGTCCTTTCCTAATAAAATGAGGAAATTGC
ATCGCATTGTCTGAGTAGGTGTCATTCTATTCTGGGGGGTGGGGT
GGGGCAGGACAGCAAGGGGGAGGATTGGGAAGACAATAGCAGGCA
TGCTGGGGATGCGGTGGGCTCTATGGAAGCTTGAATTCAGCTGAC
GTGCCTCGGACCGCTAGGAACCCCTAGTGATGGAGTTGGCCACTC
CCTCTCTGCGCGCTCGCTCGCTCACTGAGGCCGGGCGACCAAAGG
TCGCCCGACGCCCGGGCTTTGCCCGGGCGGCCTCAGTGAGCGAGC
GAGCGCGCAGCTGCCTGCAGGGGCGCCTGATGCGGTATTTTCTCC
TTACGCATCTGTGCGGTATTTCACACCGCATACGTCAAAGCAACC
ATAGTACGCGCCCTGTAGCGGCGCATTAAGCGCGGCGGGTGTGGT
GGTTACGCGCAGCGTGACCGCTACACTTGCCAGCGCCCTAGCGCC
CGCTCCTTTCGCTTTCTTCCCTTCCTTTCTCGCCACGTTCGCCGG
CTTTCCCCGTCAAGCTCTAAATCGGGGGCTCCCTTTAGGGTTCCG
ATTTAGTGCTTTACGGCACCTCGACCCCAAAAAACTTGATTTGGG
TGATGGTTCACGTAGTGGGCCATCGCCCTGATAGACGGTTTTTCG
CCCTTTGACGTTGGAGTCCACGTTCTTTAATAGTGGACTCTTGTT
CCAAACTGGAACAACACTCAACCCTATCTCGGGCTATTCTTTTGA
TTTATAAGGGATTTTGCCGATTTCGGCCTATTGGTTAAAAAATGA
GCTGATTTAACAAAAATTTAACGCGAATTTTAACAAAATATTAAC
GTTTACAATTTTATGGTGCACTCTCAGTACAATCTGCTCTGATGC
CGCATAGTTAAGCCAGCCCCGACACCCGCCAACACCCGCTGACGC
GCCCTGACGGGCTTGTCTGCTCCCGGCATCCGCTTACAGACAAGC
TGTGACCGTCTCCGGGAGCTGCATGTGTCAGAGGTTTTCACCGTC
ATCACCGAAACGCGCGAGACGAAAGGGCCTCGTGATACGCCTATT
TTTATAGGTTAATGTCATGAACAATAAAACTGTCTGCTTACATAA
ACAGTAATACAAGGGGTGTTATGAGCCATATTCAACGGGAAACGT
CGAGGCCGCGATTAAATTCCAACATGGATGCTGATTTATATGGGT
ATAAATGGGCTCGCGATAATGTCGGGCAATCAGGTGCGACAATCT
ATCGCTTGTATGGGAAGCCCGATGCGCCAGAGTTGTTTCTGAAAC
ATGGCAAAGGTAGCGTTGCCAATGATGTTACAGATGAGATGGTCA
GACTAAACTGGCTGACGGAATTTATGCCTCTTCCGACCATCAAGC
ATTTTATCCGTACTCCTGATGATGCATGGTTACTCACCACTGCGA
TCCCCGGAAAAACAGCATTCCAGGTATTAGAAGAATATCCTGATT
CAGGTGAAAATATTGTTGATGCGCTGGCAGTGTTCCTGCGCCGGT
TGCATTCGATTCCTGTTTGTAATTGTCCTTTTAACAGCGATCGCG
TATTTCGTCTCGCTCAGGCGCAATCACGAATGAATAACGGTTTGG
TTGATGCGAGTGATTTTGATGACGAGCGTAATGGCTGGCCTGTTG
AACAAGTCTGGAAAGAAATGCATAAACTTTTGCCATTCTCACCGG
ATTCAGTCGTCACTCATGGTGATTTCTCACTTGATAACCTTATTT
TTGACGAGGGGAAATTAATAGGTTGTATTGATGTTGGACGAGTCG
GAATCGCAGACCGATACCAGGATCTTGCCATCCTATGGAACTGCC
TCGGTGAGTTTTCTCCTTCATTACAGAAACGGCTTTTTCAAAAAT
ATGGTATTGATAATCCTGATATGAATAAATTGCAGTTTCATTTGA
TGCTCGATGAGTTTTTCTAATCTCATGACCAAAATCCCTTAACGT
GAGTTTTCGTTCCACTGAGCGTCAGACCCCGTAGAAAAGATCAAA
GGATCTTCTTGAGATCCTTTTTTTCTGCGCGTAATCTGCTGCTTG
CAAACAAAAAAACCACCGCTACCAGCGGTGGTTTGTTTGCCGGAT
CAAGAGCTACCAACTCTTTTTCCGAAGGTAACTGGCTTCAGCAGA
GCGCAGATACCAAATACTGTCCTTCTAGTGTAGCCGTAGTTAGGC
CACCACTTCAAGAACTCTGTAGCACCGCCTACATACCTCGCTCTG
CTAATCCTGTTACCAGTGGCTGCTGCCAGTGGCGATAAGTCGTGT
CTTACCGGGTTGGACTCAAGACGATAGTTACCGGATAAGGCGCAG
CGGTCGGGCTGAACGGGGGGTTCGTGCACACAGCCCAGCTTGGAG
CGAACGACCTACACCGAACTGAGATACCTACAGCGTGAGCTATGA
GAAAGCGCCACGCTTCCCGAAGGGAGAAAGGCGGACAGGTATCCG
GTAAGCGGCAGGGTCGGAACAGGAGAGCGCACGAGGGAGCTTCCA
GGGGGAAACGCCTGGTATCTTTATAGTCCTGTCGGGTTTCGCCAC
CTCTGACTTGAGCGTCGATTTTTGTGATGCTCGTCAGGGGGGCGG
AGCCTATGGAAAAACGCCAGCAACGCGGCCTTTTTACGGTTCCTG
GCCTTTTGCTGGCCTTTTGCTCACATGT

TABLE 9
Components of Construct Sequence (SEQ ID NO: 100)

| Components | Position in construct |
|---|---|
| 5′ITR | 12-130 |
| Cloning site | 121-147 |
| BACE2 promoter | 148-1551 |
| hGJB2 minimal promoter | 1552-1679 |
| Cloning site | 1680-1688 |
| Synthetic barcode | 1689-1696 |
| 5′UTR | 1697-2058 |
| GJB2 (exon2) | 2070-2747 |
| 3xFLAG | 2760-2825 |
| 3′UTR (exon2) | 2829-4235 |
| bGHpA | 4255-4481 |
| Cloning site | 4482-4515 |
| 3′ITR | 4516-4645 |

Exemplary Construct sequence
(SEQ ID NO: 101)
CCTGCAGGCAGCTGCGCGCTCGCTCGCTCACTGAGGCCGCCCGGG
CGTCGGGCGACCTTTGGTCGCCCGGCCTCAGTGAGCGAGCGAGCG
CGCAGAGAGGGAGTGGCCAACTCCATCACTAGGGGTTCCTGCGGC
CGCACGCGTGGTGAAGAAACCTGCATTTCTTACACTTCAGTGTAC
TTTCCCCATATTTAACTCCAAGATTTTTGTTAATTTGTTTGGTTT
TCCTTTCTCAAACAAAATTATGCTCAGACTGAAAACCCTAGATTT
GTTCCCTATTGCATCTTCATTTCTTCCCAAACATTCCATAAAACG
TGACCTACATTAAGTTAGCAAGTTAAGTCTGAAAGCGTCTACCTT
CCCTGGGGAGGGGGAAGGTGTAGGCAGGGCAGAGATTTGTAGTCC
AGCCCTCTTGCCACAAATTATGAATTAGAGAGGAATGACTTTGCT
TTTTTAATGATCTCCAGAGAATTTTCCATCATTTCCCTCTCTTCA
CCCAGCTCCTTTGCAACCACTGCCAGAGAAGTCTTCCTTTAGCTT
CTTAAACATCGATCCTAAAACACTTCCAGACACCTGTGCTGCTCC
TTTCAGTTCCCATGGAGATTAGGCTGTGTAACAATCTCGCAAAGA
CGTTCCCCTCCGTCTCCTCATCCTCTTTTCAAACCCTTTTACGAT
TTCCCATCTCACTCAGCATGACAGTCAAAGTCCCTGTGATGGCCA
ACTTCTGCATCACCTAGCCAGTCTGCCACCGCCAAAACTCTCCAG
CCTCATCTTACACTTGTTCTCTGCTTGGAATCTTCCCTCCCCTCC
TTGAGGAACTTTCTCAAATGTCACCTTCCCTCAATACTCCCCCTC
CTCCATTTAAAACTATAAACTTCCAACTCTCTAAGCCCCTAAAGT
ACTCTATATTTAACTTATTGTATAAACTACTGTCCCTACTTGTAA
GTTCCAAGATTGCAGGGATTCACCCGCTTTGTTCACTGCTGTCTG
CCAAGGTCTAGAACAGTGCAAGTTACCCAACAGGAGTTCAATAAA
CAGCCATTCATTTAACAAATATTTGCTGAGCACTTCGTCCCGTCC
AAGTTTGTTAAATCAAGACAAATAAGACACCGTCCCTGCCTTTAA
CGCACCAGATGGAGAAATGCACCACAGACATAAATGTGCAATACA
GGCCTGACACTACGGCCACAAGCAAGTCAAAGAACGTGCCAAAAG
TTCAGAGGAAGAAGCCTCGGCTTCGCCTTTCGGGAGACCAGTCCA
GCTTTCCACCATCACGCTGCTCATCAGGGACCATCTCCGGGGGTC
TCCTCTAGACCCCAAGGGAGGAGCGGGTCCCGCCCGCCATTCCCA
GGTCTCAGAGTTTACTTGTCCAGAGATGCAACTTCCGGCCTCTTC
AGGCCGGGCAAGATTTAAGGAAAGAAAAGAAACATAAGGACCTCC
GTTCTTCGGTCTCCGTCCCCTCCCCTTCCCCCGCGTGCCCCACCT
GTTCCCGGCGTCCCCTTCGGCTACTCCCGGCGTTTGCGCAAGCGG
TCCCACGTGGGCTCGGGCGGGGCTAGCGCCGCGGCGGGGGCTGGG
CACGCCCCTAGCGCATAGCTGGCTTCTGATTGGCTTTCCAAGCTC
TGAGGACCCAGAGGCCGGGCGCGCTCCGCCCGCGGCGCCGCCCCC
TCCGTAACTTTCCCAGTCTCCGAGGGAAGAGGCGGGGTGTGGGGT
GCGGTTAAAAGGCGCCACGGCGGGAGACAGGTCTCACCGGTGCCA
AAGCGTTGCGGCCCCGCAGCGCCCGCGCGCTCCTCTCCCCGACTC
GGAGCCCCTCGGCGGCGCCCGGCCCAGGACCCGCCTAGGAGCGCA
GGAGCCCCAGCGCAGAGACCCCAACGCCGAGACCCCCGCCCCGGC
CCCGCCGCGCTTCCTCCCGACGCAGTTTAGGACCCTTGTTCGCGA
AGAGGTGGTGTGCGGCTGAGACCCGCGTCCTCAGGACGGTTCCAT
CAGTGCCTCGATCCTGCCCCACTGGAGGAGGAAGGCAGCCCGAAC
AGCGCTCACCTAACTAACAGCTGCTGAGAGCTGGGTTCCGTGGCC
ATGCACCTGGGACTGCCTTGAGAAGCGTGAGCAAACCGCCCAGAG
TAGAAGCGCTAGCCACCATGGATTGGGGCACGCTGCAGACGATCC
TGGGGGGTGTGAACAAACACTCCACCAGCATTGGAAAGATCTGGC
TCACCGTCCTCTTCATTTTTCGCATTATGATCCTCGTTGTGGCTG
CAAAGGAGGTGTGGGGAGATGAGCAGGCCGACTTTGTCTGCAACA
CCCTGCAGCCAGGCTGCAAGAACGTGTGCTACGATCACTACTTCC
CCATCTCCCACATCCGGCTATGGGCCCTGCAGCTGATCTTCGTGT
CCACGCCAGCGCTCCTAGTGGCCATGCACGTGGCCTACCGGAGAC
ATGAGAAGAAGAGGAAGTTCATCAAGGGGGAGATAAAGAGTGAAT
TTAAGGACATCGAGGAGATCAAAACCCAGAAGGTCCGCATCGAAG
GCTCCCTGTGGTGGACCTACACAAGCAGCATCTTCTTCCGGGTCA
TCTTCGAAGCCGCCTTCATGTACGTCTTCTATGTCATGTACGACG
GCTTCTCCATGCAGCGGCTGGTGAAGTGCAACGCCTGGCCTTGTC
CCAACACTGTGGACTGCTTTGTGTCCCGGCCCACGGAGAAGACTG
TCTTCACAGTGTTCATGATTGCAGTGTCTGGAATTTGCATCCTGC
TGAATGTCACTGAATTGTGTTATTTGCTAATTAGATATTGTTCTG
GGAAGTCAAAAAAGCCAGTTGGATCCCGGGCTGACTACAAAGACC
ATGACGGTGATTATAAAGATCATGACATCGACTACAAGGATGACG
ATGACAAGTAAGAAATAGACAGCATGAGAGGGATGAGGCAACCCG
TGCTCAGCTGTCAAGGCTCAGTCGCTAGCATTTCCCAACACAAAG
ATTCTGACCTTAAATGCAACCATTTGAAACCCCTGTAGGCCTCAG
GTGAAACTCCAGATGCCACAATGGAGCTCTGCTCCCCTAAAGCCT
CAAAACAAAGGCCTAATTCTATGCCTGTCTTAATTTTCTTTCACT
TAAGTTAGTTCCACTGAGACCCCAGGCTGTTAGGGGTTATTGGTG
TAAGGTACTTTCATATTTTAAACAGAGGATATCGGCATTTGTTTC
TTTCTCTGAGGACAAGAGAAAAAAGCCAGGTTCCACAGAGGACAC
AGAGAAGGTTTGGGTGTCCTCCTGGGGTTCTTTTTGCCAACTTTC
CCCACGTTAAAGGTGAACATTGGTTCTTTCATTTGCTTTGGAAGT
TTTAATCTCTAACAGTGGACAAAGTTACCAGTGCCTTAAACTCTG
TTACACTTTTTGGAAGTGAAAACTTTGTAGTATGATAGGTTATTT
TGATGTAAAGATGTTCTGGATACCATTATATGTTCCCCCTGTTTC
AGAGGCTCAGATTGTAATATGTAAATGGTATGTCATTCGCTACTA
TGATTTAATTTGAAATATGGTCTTTTGGTTATGAATACTTTGCAG
CACAGCTGAGAGGCTGTCTGTTGTATTCATTGTGGTCATAGCACC
TAACAACATTGTAGCCTCAATCGAGTGAGACAGACTAGAAGTTCC
TAGTGATGGCTTATGATAGCAAATGGCCTCATGTCAAATATTTAG
ATGTAATTTTGTGTAAGAAATACAGACTGGATGTACCACCAACTA
CTACCTGTAATGACAGGCCTGTCCAACACATCTCCCTTTTCCATG
ACTGTGGTAGCCAGCATCGGAAAGAACGCTGATTTAAAGAGGTCG
CTTGGGAATTTTATTGACACAGTACCATTTAATGGGGAGGACAAA
ATGGGGCAGGGGAGGGAGAAGTTTCTGTCGTTAAAAACAGATTTG
GAAAGACTGGACTCTAAAGTCTGTTGATTAAAGATGAGCTTTGTC
TACTTCAAAAGTTTGTTTGCTTACCCCTTCAGCCTCCAATTTTTT
AAGTGAAAATATAGCTAATAACATGTGAAAAGAATAGAAGCTAAG
GTTTAGATAAATATTGAGCAGATCTATAGGAAGATTGAACCTGAA
TATTGCCATTATGCTTGACATGGTTTCCAAAAAATGGTACTCCAC
ATATTTCAGTGAGGGTAAGTATTTTCCTGTTGTCAAGAATAGCAT
TGTAAAAGCATTTTGTAATAATAAAGAATAGCTTTAATGATATGC
TTGTAACTAAAATAATTTTGTAATGTATCAAATACATTTAAAACA
TTAAAATATAATCTCTATAATAAGAGCTCGCTGATCAGCCTCGAC
TGTGCCTTCTAGTTGCCAGCCATCTGTTGTTTGCCCCTCCCCCGT
GCCTTCCTTGACCCTGGAAGGTGCCACTCCCACTGTCCTTTCCTA
ATAAAATGAGGAAATTGCATCGCATTGTCTGAGTAGGTGTCATTC
TATTCTGGGGGGTGGGGTGGGGCAGGACAGCAAGGGGGAGGATTG
GGAAGACAATAGCAGGCATGCTGGGGATGCGGTGGGCTCTATGGA
AGCTTGAATTCAGCTGACGTGCCTCGGACCGCTAGGAACCCCTAG
TGATGGAGTTGGCCACTCCCTCTCTGCGCGCTCGCTCGCTCACTG
AGGCCGGGCGACCAAAGGTCGCCCGACGCCCGGGCTTTGCCCGGG
CGGCCTCAGTGAGCGAGCGAGCGCGCAGCTGCCTGCAGGGGCGCC
TGATGCGGTATTTTCTCCTTACGCATCTGTGCGGTATTTCACACC
GCATACGTCAAAGCAACCATAGTACGCGCCCTGTAGCGGCGCATT
AAGCGCGGCGGGTGTGGTGGTTACGCGCAGCGTGACCGCTACACT
TGCCAGCGCCCTAGCGCCCGCTCCTTTCGCTTTCTTCCCTTCCTT
TCTCGCCACGTTCGCCGGCTTTCCCCGTCAAGCTCTAAATCGGGG
GCTCCCTTTAGGGTTCCGATTTAGTGCTTTACGGCACCTCGACCC
CAAAAAACTTGATTTGGGTGATGGTTCACGTAGTGGGCCATCGCC
CTGATAGACGGTTTTTCGCCCTTTGACGTTGGAGTCCACGTTCTT
TAATAGTGGACTCTTGTTCCAAACTGGAACAACACTCAACCCTAT
CTCGGGCTATTCTTTTGATTTATAAGGGATTTTGCCGATTTCGGC
CTATTGGTTAAAAAATGAGCTGATTTAACAAAAATTTAACGCGAA
TTTTAACAAAATATTAACGTTTACAATTTTATGGTGCACTCTCAG
TACAATCTGCTCTGATGCCGCATAGTTAAGCCAGCCCCGACACCC
GCCAACACCCGCTGACGCGCCCTGACGGGCTTGTCTGCTCCCGGC
ATCCGCTTACAGACAAGCTGTGACCGTCTCCGGGAGCTGCATGTG
TCAGAGGTTTTCACCGTCATCACCGAAACGCGCGAGACGAAAGGG
CCTCGTGATACGCCTATTTTTATAGGTTAATGTCATGAACAATAA
AACTGTCTGCTTACATAAACAGTAATACAAGGGGTGTTATGAGCC
ATATTCAACGGGAAACGTCGAGGCCGCGATTAAATTCCAACATGG
ATGCTGATTTATATGGGTATAAATGGGCTCGCGATAATGTCGGGC
AATCAGGTGCGACAATCTATCGCTTGTATGGGAAGCCCGATGCGC
CAGAGTTGTTTCTGAAACATGGCAAAGGTAGCGTTGCCAATGATG
TTACAGATGAGATGGTCAGACTAAACTGGCTGACGGAATTTATGC
CTCTTCCGACCATCAAGCATTTTATCCGTACTCCTGATGATGCAT
GGTTACTCACCACTGCGATCCCCGGAAAAACAGCATTCCAGGTAT
TAGAAGAATATCCTGATTCAGGTGAAAATATTGTTGATGCGCTGG
CAGTGTTCCTGCGCCGGTTGCATTCGATTCCTGTTTGTAATTGTC
CTTTTAACAGCGATCGCGTATTTCGTCTCGCTCAGGCGCAATCAC
GAATGAATAACGGTTTGGTTGATGCGAGTGATTTTGATGACGAGC
GTAATGGCTGGCCTGTTGAACAAGTCTGGAAAGAAATGCATAAAC
TTTTGCCATTCTCACCGGATTCAGTCGTCACTCATGGTGATTTCT
CACTTGATAACCTTATTTTTGACGAGGGGAAATTAATAGGTTGTA
TTGATGTTGGACGAGTCGGAATCGCAGACCGATACCAGGATCTTG
CCATCCTATGGAACTGCCTCGGTGAGTTTTCTCCTTCATTACAGA
AACGGCTTTTTCAAAAATATGGTATTGATAATCCTGATATGAATA
AATTGCAGTTTCATTTGATGCTCGATGAGTTTTTCTAATCTCATG
ACCAAAATCCCTTAACGTGAGTTTTCGTTCCACTGAGCGTCAGAC
CCCGTAGAAAAGATCAAAGGATCTTCTTGAGATCCTTTTTTTCTG
CGCGTAATCTGCTGCTTGCAAACAAAAAAACCACCGCTACCAGCG
GTGGTTTGTTTGCCGGATCAAGAGCTACCAACTCTTTTTCCGAAG
GTAACTGGCTTCAGCAGAGCGCAGATACCAAATACTGTCCTTCTA
GTGTAGCCGTAGTTAGGCCACCACTTCAAGAACTCTGTAGCACCG
CCTACATACCTCGCTCTGCTAATCCTGTTACCAGTGGCTGCTGCC
AGTGGCGATAAGTCGTGTCTTACCGGGTTGGACTCAAGACGATAG
TTACCGGATAAGGCGCAGCGGTCGGGCTGAACGGGGGGTTCGTGC
ACACAGCCCAGCTTGGAGCGAACGACCTACACCGAACTGAGATAC
CTACAGCGTGAGCTATGAGAAAGCGCCACGCTTCCCGAAGGGAGA
AAGGCGGACAGGTATCCGGTAAGCGGCAGGGTCGGAACAGGAGAG
CGCACGAGGGAGCTTCCAGGGGGAAACGCCTGGTATCTTTATAGT
CCTGTCGGGTTTCGCCACCTCTGACTTGAGCGTCGATTTTTGTGA
TGCTCGTCAGGGGGGCGGAGCCTATGGAAAAACGCCAGCAACGCG
GCCTTTTTACGGTTCCTGGCCTTTTGCTGGCCTTTTGCTCACATG
T

TABLE 10
Components of Construct Sequence (SEQ ID NO: 101)

| Components | Position in construct |
|---|---|
| 5′ITR | 12-130 |
| Cloning site | 131-147 |
| DBI2 promoter | 148-1614 |
| hGJB2 minimal promoter | 1615-1742 |
| Cloning site | 1743-1751 |
| Synthetic barcode | 1752-1759 |
| 5′UTR | 1760-2121 |
| GJB2 (exon2) | 2133-2810 |
| 3xFLAG | 2823-2888 |
| 3′UTR (exon2) | 2892-4298 |
| bGHpA | 4320-4544 |
| Cloning site | 4545-4578 |
| 3′ITR | 4579-4708 |

Exemplary Construct sequence
(SEQ ID NO: 102)
CCTGCAGGCAGCTGCGCGCTCGCTCGCTCACTGAGGCCGCCCGGG
CGTCGGGCGACCTTTGGTCGCCCGGCCTCAGTGAGCGAGCGAGCG
CGCAGAGAGGGAGTGGCCAACTCCATCACTAGGGGTTCCTGCGGC
CGCACGCGTGGTTACCATTCTGCCTTTCACCTGATGTTGCTATCC
TCCTCCCTCTTGTTTCCTTCCACCCATCCTTTCCCTCCCACATTA
CTCTCTTATCCCACCCTATTTTACAACCAGTAGCCTAGGGAAAAG
AGCATAGCTCAAATGAGGAAGAAGGCAGGACAGGCAGTCATGGCT
TAGCTGGACTGAGCTGCAGTGCTTCTCCTTCTGGGGAAGGGGGTG
CACTGTCATCTGCTACTGACACATCCCTCCAAGGCACTCAGCCCT
GCAGGGAGCAACCTGATTCTATGACTGACATCTAATCTTCACATT
CACCTTGCAGGAAGGCAAGAAGTGATCCCAGCCTCCAGATGGAAA
GATCAAGGCCCAGAGAAGGTCAGTGGTGGTTGGAGGCCTGAGGTC
ACACAGCAGCCAAGTCTGGAGTCACTAGTCAAGGTGACCTTGACT
AGCCACCCCACCTCCCCTTCCCTGCCCCACCATGGCCCTGGGAGA
TCTGTTGTCCTGTGAGGGAAAGGGGCTCCAGGCTGGGCTGCATCT
GAAGCCCCTAGATCCAGAGACTTCATTTCTTAGGCTATCTATAAA
ATCCACCTTCCTTTCTTTTCCCAGGACCCCCATACCCTGCTCCCA
GCATCGTCTGCCTCAGCTAAGCCATGGGGATTGAGAGACCAGGCC
TGGTGCCCAGATAAACTGACCCTGGGTGAGGGGACAGGGGCCCAG
AATGGGCAGGTAGAGACTGAATACTGAAGAAGAATCCTCTGGAGT
CTGTTAGCAGAAGCAGATGGGCCTTGCCTGACTATTGGCAGGCGG
ACCTGGTGGTCAGACCTCAGTGATCCTCAGGGACCAGTGAATATT
TCAGGCTGGGGCTGAGCATCACCTGCTCCCTTGGCCCCACTTATA
GGGCAAAGGGGAGTCTACCAGCCTACTCACTGATGACAAACTGGA
AAAGTTTGTCCTGTCTCTGCTCTGGCCCCACCTCGCCCTCTCCCC
TACTTGGAAGTTCCTTTCCTGAACCACTGACTGCCAAAGCTTGAG
GGATTAAATAAATCATCTGGCCCAACCTCCTACCATAGAGTTGGG
AACACTGAAGAAAAGAGACTGGCCCAAGGTCACAGAGAAGGCAGG
GTGAACACTGTCACAGGGAGAGCCAGTGTAGAATAATGGTTAAGC
CACGCAAGCTCTAGAACCACTCTATCTGAGTGCAAATCCTGGCTG
TCATCTGGTACTTGCTTCCTGGAACACATCTGGCCTCAGACTCCT
GAGGCCAAGACACACTCCCTGCCCTAAGACTTGCTGGTTCTATGG
CAGGCAGAGGCAGAAAGAGCCCCACCATCATTCCCAGCAAATGGG
AAAAGTTCCCAGTTGCAGATATTAGGGGTGGGATGGGGCGGGGGT
AGTCAGCAACCATAGACTTAGACCCTGAAGAGGCAAAAAAGGAGG
GCCATGTTCTTGGGTCAGCAGAGCTTCTACTCAGCTTCTTCAGCC
TCTAGCTCTTTCCTGGTGCTAGTAGCACATTCTCTAGTGGAGGCA
TCCAGATGGCAGGGAGGGTCCAGGAAACAGCTGAACATGCTGAGC
AGGCCTCCCTTGTCCCCGCTCCCCATGGCCCCATGGATCATCCGG
TGCTGCAGCTCATCTCATTGGCTGGCTTCTGGTTACTCATCTCTC
CTCTTCTCCATCTTCCCAGCCTGTGGTTGCCGTGGAAACATAGAA
CAGTGACCTCACCATAGGATGAGGGCTGGGGAGATGCTGTTCTTG
GCAGGCGCTAAGCTCTGAGGACCCAGAGGCCGGGCGCGCTCCGCC
CGCGGCGCCGCCCCCTCCGTAACTTTCCCAGTCTCCGAGGGAAGA
GGCGGGGTGTGGGGTGCGGTTAAAAGGCGCCACGGCGGGAGACAG
GTCTCACCGGTCCATCCACGTTGCGGCCCCGCAGCGCCCGCGCGC
TCCTCTCCCCGACTCGGAGCCCCTCGGCGGCGCCCGGCCCAGGAC
CCGCCTAGGAGCGCAGGAGCCCCAGCGCAGAGACCCCAACGCCGA
GACCCCCGCCCCGGCCCCGCCGCGCTTCCTCCCGACGCAGTTTAG
GACCCTTGTTCGCGAAGAGGTGGTGTGCGGCTGAGACCCGCGTCC
TCAGGACGGTTCCATCAGTGCCTCGATCCTGCCCCACTGGAGGAG
GAAGGCAGCCCGAACAGCGCTCACCTAACTAACAGCTGCTGAGAG
CTGGGTTCCGTGGCCATGCACCTGGGACTGCCTTGAGAAGCGTGA
GCAAACCGCCCAGAGTAGAAGCGCTAGCCACCATGGATTGGGGCA
CGCTGCAGACGATCCTGGGGGGTGTGAACAAACACTCCACCAGCA
TTGGAAAGATCTGGCTCACCGTCCTCTTCATTTTTCGCATTATGA
TCCTCGTTGTGGCTGCAAAGGAGGTGTGGGGAGATGAGCAGGCCG
ACTTTGTCTGCAACACCCTGCAGCCAGGCTGCAAGAACGTGTGCT
ACGATCACTACTTCCCCATCTCCCACATCCGGCTATGGGCCCTGC
AGCTGATCTTCGTGTCCACGCCAGCGCTCCTAGTGGCCATGCACG
TGGCCTACCGGAGACATGAGAAGAAGAGGAAGTTCATCAAGGGGG
AGATAAAGAGTGAATTTAAGGACATCGAGGAGATCAAAACCCAGA
AGGTCCGCATCGAAGGCTCCCTGTGGTGGACCTACACAAGCAGCA
TCTTCTTCCGGGTCATCTTCGAAGCCGCCTTCATGTACGTCTTCT
ATGTCATGTACGACGGCTTCTCCATGCAGCGGCTGGTGAAGTGCA
ACGCCTGGCCTTGTCCCAACACTGTGGACTGCTTTGTGTCCCGGC
CCACGGAGAAGACTGTCTTCACAGTGTTCATGATTGCAGTGTCTG
GAATTTGCATCCTGCTGAATGTCACTGAATTGTGTTATTTGCTAA
TTAGATATTGTTCTGGGAAGTCAAAAAAGCCAGTTGGATCCCGGG
CTGACTACAAAGACCATGACGGTGATTATAAAGATCATGACATCG
ACTACAAGGATGACGATGACAAGTAAGAAATAGACAGCATGAGAG
GGATGAGGCAACCCGTGCTCAGCTGTCAAGGCTCAGTCGCTAGCA
TTTCCCAACACAAAGATTCTGACCTTAAATGCAACCATTTGAAAC
CCCTGTAGGCCTCAGGTGAAACTCCAGATGCCACAATGGAGCTCT
GCTCCCCTAAAGCCTCAAAACAAAGGCCTAATTCTATGCCTGTCT
TAATTTTCTTTCACTTAAGTTAGTTCCACTGAGACCCCAGGCTGT
TAGGGGTTATTGGTGTAAGGTACTTTCATATTTTAAACAGAGGAT
ATCGGCATTTGTTTCTTTCTCTGAGGACAAGAGAAAAAAGCCAGG
TTCCACAGAGGACACAGAGAAGGTTTGGGTGTCCTCCTGGGGTTC
TTTTTGCCAACTTTCCCCACGTTAAAGGTGAACATTGGTTCTTTC
ATTTGCTTTGGAAGTTTTAATCTCTAACAGTGGACAAAGTTACCA
GTGCCTTAAACTCTGTTACACTTTTTGGAAGTGAAAACTTTGTAG
TATGATAGGTTATTTTGATGTAAAGATGTTCTGGATACCATTATA
TGTTCCCCCTGTTTCAGAGGCTCAGATTGTAATATGTAAATGGTA
TGTCATTCGCTACTATGATTTAATTTGAAATATGGTCTTTTGGTT
ATGAATACTTTGCAGCACAGCTGAGAGGCTGTCTGTTGTATTCAT
TGTGGTCATAGCACCTAACAACATTGTAGCCTCAATCGAGTGAGA
CAGACTAGAAGTTCCTAGTGATGGCTTATGATAGCAAATGGCCTC
ATGTCAAATATTTAGATGTAATTTTGTGTAAGAAATACAGACTGG
ATGTACCACCAACTACTACCTGTAATGACAGGCCTGTCCAACACA
TCTCCCTTTTCCATGACTGTGGTAGCCAGCATCGGAAAGAACGCT
GATTTAAAGAGGTCGCTTGGGAATTTTATTGACACAGTACCATTT
AATGGGGAGGACAAAATGGGGCAGGGGAGGGAGAAGTTTCTGTCG
TTAAAAACAGATTTGGAAAGACTGGACTCTAAAGTCTGTTGATTA
AAGATGAGCTTTGTCTACTTCAAAAGTTTGTTTGCTTACCCCTTC
AGCCTCCAATTTTTTAAGTGAAAATATAGCTAATAACATGTGAAA
AGAATAGAAGCTAAGGTTTAGATAAATATTGAGCAGATCTATAGG
AAGATTGAACCTGAATATTGCCATTATGCTTGACATGGTTTCCAA
AAAATGGTACTCCACATATTTCAGTGAGGGTAAGTATTTTCCTGT
TGTCAAGAATAGCATTGTAAAAGCATTTTGTAATAATAAAGAATA
GCTTTAATGATATGCTTGTAACTAAAATAATTTTGTAATGTATCA
AATACATTTAAAACATTAAAATATAATCTCTATAATAAGAGCTCG
CTGATCAGCCTCGACTGTGCCTTCTAGTTGCCAGCCATCTGTTGT
TTGCCCCTCCCCCGTGCCTTCCTTGACCCTGGAAGGTGCCACTCC
CACTGTCCTTTCCTAATAAAATGAGGAAATTGCATCGCATTGTCT
GAGTAGGTGTCATTCTATTCTGGGGGGTGGGGTGGGGCAGGACAG
CAAGGGGGAGGATTGGGAAGACAATAGCAGGCATGCTGGGGATGC
GGTGGGCTCTATGGAAGCTTGAATTCAGCTGACGTGCCTCGGACC
GCTAGGAACCCCTAGTGATGGAGTTGGCCACTCCCTCTCTGCGCG
CTCGCTCGCTCACTGAGGCCGGGCGACCAAAGGTCGCCCGACGCC
CGGGCTTTGCCCGGGCGGCCTCAGTGAGCGAGCGAGCGCGCAGCT
GCCTGCAGGGGCGCCTGATGCGGTATTTTCTCCTTACGCATCTGT
GCGGTATTTCACACCGCATACGTCAAAGCAACCATAGTACGCGCC
CTGTAGCGGCGCATTAAGCGCGGCGGGTGTGGTGGTTACGCGCAG
CGTGACCGCTACACTTGCCAGCGCCCTAGCGCCCGCTCCTTTCGC
TTTCTTCCCTTCCTTTCTCGCCACGTTCGCCGGCTTTCCCCGTCA
AGCTCTAAATCGGGGGCTCCCTTTAGGGTTCCGATTTAGTGCTTT
ACGGCACCTCGACCCCAAAAAACTTGATTTGGGTGATGGTTCACG
TAGTGGGCCATCGCCCTGATAGACGGTTTTTCGCCCTTTGACGTT
GGAGTCCACGTTCTTTAATAGTGGACTCTTGTTCCAAACTGGAAC
AACACTCAACCCTATCTCGGGCTATTCTTTTGATTTATAAGGGAT
TTTGCCGATTTCGGCCTATTGGTTAAAAAATGAGCTGATTTAACA
AAAATTTAACGCGAATTTTAACAAAATATTAACGTTTACAATTTT
ATGGTGCACTCTCAGTACAATCTGCTCTGATGCCGCATAGTTAAG
CCAGCCCCGACACCCGCCAACACCCGCTGACGCGCCCTGACGGGC
TTGTCTGCTCCCGGCATCCGCTTACAGACAAGCTGTGACCGTCTC
CGGGAGCTGCATGTGTCAGAGGTTTTCACCGTCATCACCGAAACG
CGCGAGACGAAAGGGCCTCGTGATACGCCTATTTTTATAGGTTAA
TGTCATGAACAATAAAACTGTCTGCTTACATAAACAGTAATACAA
GGGGTGTTATGAGCCATATTCAACGGGAAACGTCGAGGCCGCGAT
TAAATTCCAACATGGATGCTGATTTATATGGGTATAAATGGGCTC
GCGATAATGTCGGGCAATCAGGTGCGACAATCTATCGCTTGTATG
GGAAGCCCGATGCGCCAGAGTTGTTTCTGAAACATGGCAAAGGTA
GCGTTGCCAATGATGTTACAGATGAGATGGTCAGACTAAACTGGC
TGACGGAATTTATGCCTCTTCCGACCATCAAGCATTTTATCCGTA
CTCCTGATGATGCATGGTTACTCACCACTGCGATCCCCGGAAAAA
CAGCATTCCAGGTATTAGAAGAATATCCTGATTCAGGTGAAAATA
TTGTTGATGCGCTGGCAGTGTTCCTGCGCCGGTTGCATTCGATTC
CTGTTTGTAATTGTCCTTTTAACAGCGATCGCGTATTTCGTCTCG
CTCAGGCGCAATCACGAATGAATAACGGTTTGGTTGATGCGAGTG
ATTTTGATGACGAGCGTAATGGCTGGCCTGTTGAACAAGTCTGGA
AAGAAATGCATAAACTTTTGCCATTCTCACCGGATTCAGTCGTCA
CTCATGGTGATTTCTCACTTGATAACCTTATTTTTGACGAGGGGA
AATTAATAGGTTGTATTGATGTTGGACGAGTCGGAATCGCAGACC
GATACCAGGATCTTGCCATCCTATGGAACTGCCTCGGTGAGTTTT
CTCCTTCATTACAGAAACGGCTTTTTCAAAAATATGGTATTGATA
ATCCTGATATGAATAAATTGCAGTTTCATTTGATGCTCGATGAGT
TTTTCTAATCTCATGACCAAAATCCCTTAACGTGAGTTTTCGTTC
CACTGAGCGTCAGACCCCGTAGAAAAGATCAAAGGATCTTCTTGA
GATCCTTTTTTTCTGCGCGTAATCTGCTGCTTGCAAACAAAAAAA
CCACCGCTACCAGCGGTGGTTTGTTTGCCGGATCAAGAGCTACCA
ACTCTTTTTCCGAAGGTAACTGGCTTCAGCAGAGCGCAGATACCA
AATACTGTCCTTCTAGTGTAGCCGTAGTTAGGCCACCACTTCAAG
AACTCTGTAGCACCGCCTACATACCTCGCTCTGCTAATCCTGTTA
CCAGTGGCTGCTGCCAGTGGCGATAAGTCGTGTCTTACCGGGTTG
GACTCAAGACGATAGTTACCGGATAAGGCGCAGCGGTCGGGCTGA
ACGGGGGGTTCGTGCACACAGCCCAGCTTGGAGCGAACGACCTAC
ACCGAACTGAGATACCTACAGCGTGAGCTATGAGAAAGCGCCACG
CTTCCCGAAGGGAGAAAGGCGGACAGGTATCCGGTAAGCGGCAGG
GTCGGAACAGGAGAGCGCACGAGGGAGCTTCCAGGGGGAAACGCC
TGGTATCTTTATAGTCCTGTCGGGTTTCGCCACCTCTGACTTGAG
CGTCGATTTTTGTGATGCTCGTCAGGGGGGCGGAGCCTATGGAAA
AACGCCAGCAACGCGGCCTTTTTACGGTTCCTGGCCTTTTGCTGG
CCTTTTGCTCACATGT

TABLE 11
Components of Construct Sequence (SEQ ID NO: 102)

| Components | Position in construct |
|---|---|
| 5′ITR | 12-130 |
| Cloning site | 131-147 |
| FABP3 promoter | 148-1899 |
| hGJB2 minimal promoter | 1900-2027 |
| Cloning site | 2028-2036 |
| Synthetic barcode | 2037-2044 |
| 5′UTR | 2045-2406 |
| GJB2 (exon2) | 2418-3095 |
| 3xFLAG | 3108-3173 |
| 3′UTR (exon2) | 3177-4583 |
| bGHpA | 4605-4829 |
| Cloning site | 4830-4863 |
| 3′ITR | 4864-4993 |

Exemplary Construct sequence
(SEQ ID NO: 103)
CCTGCAGGCAGCTGCGCGCTCGCTCGCTCACTGAGGCCGCCCGGG
CGTCGGGCGACCTTTGGTCGCCCGGCCTCAGTGAGCGAGCGAGCG
CGCAGAGAGGGAGTGGCCAACTCCATCACTAGGGGTTCCTGCGGC
CGCACGCGTGGTGAAACAGCAGCCATTGATGTAGCTCAGGGTTCT
GTGGATCTGTCATTTGGAGCATGTTGGTTCTCCTGTCTCAGCTGG
GCTCATTCATGCATCTGAGTTCAGCTATTGGGCAATCTGGGGAAT
GTTTTGTCCATGTGATGTGTCATCTTCTACCAGGCTAGCCTGGGC
TTCATCACATGGTATCTGGCAGGGCTCTAAGAGGGAGAGTTGAAA
CACACAAGGCCTCTTGAAGCTTAGACTCAGAATTGGCACAAGGTC
GCTTCTGGCACATTCCATTGGTCAAAGCAAGTTACAAGGCCAGCT
CACATTCAAGGATTAGGTAAGTCGATTCCACTCTTGATGAGAAGT
CTGAAGGATTTGGAACAGTGTCCACCATGCAGTAATAAACTCAAT
AAGTAGTAGCCATTATTATTCTGTTAGAGGTTGCCAGGAAAAGTT
TTATAGTGGAAAGAAATCTGAGTTTACTCTTGAGAGGTAAGTGGA
ATTTCTATTTGTAGAGAATGAAGGCCTCTCAAAAAGACACAGCCT
AACAATAGGTGCTGCAGTTTAACAGTGGAGCGTGTCCAGAACAGG
CTGCCCTTTTAGGCAAGGGCTAGTGTCTTTCAGGACAGACCCAAA
CCCCAAATACCAAAACAGAATAAAGTAGTGTCTTAGCATACTTTG
AGATCAGACTGTTTCTGCATTTCACAGTGCTGGGGGTGGGGGGGA
GGTGTGGGGGGAAGGGAAAAGCAGCATACCAATGTAGTGAAATCT
GGAAACAACAGCCAAAAAAAGTTTGCATATTGCACAGAGCACTTG
AAGATCATAAATCTATGCATGAGAAAGATGTAGTGGAAATTTTGG
GGGGGATTAGAGTTTATTTTTGTCATCTCTGTGAGACAGCTACTC
ATTCATCCAGATCACAGCTAAGAAAAAAGCTGGTCACAGAAATTA
GCAGTTTCAGCTCAGCAGCGAAGTCGCCAGCCTGTGAAGGCAGAG
AGAAATTGACTAATTAGCAATGCGCACTAAAACTTGACGGTTCTT
TATAGAGAGAGAGAAGAGAGAGGGAGAGAGAGGGAGAGGGAGGGA
GGGGGGGCTCGCTTTTTCCCCTTCTTTCTTCCAAAGATGTTTGAA
ATCGCAGTCATTTACGCTCGACAATTTTTACAATAGCCTTGAGCC
ATAATTTTGCGAGTCTCTCCAGCATCCATCCCCCTGTATGGTCTC
TCTCTACTGGCCAAGCACGACCGTTTCTCTCCCCAACCGTGGATT
TCCTATTAAGCTCTGAGGACCCAGAGGCCGGGCGCGCTCCGCCCG
CGGCGCCGCCCCCTCCGTAACTTTCCCAGTCTCCGAGGGAAGAGG
CGGGGTGTGGGGTGCGGTTAAAAGGCGCCACGGCGGGAGACAGGT
CTCACCGGTCCCGTTCTGTTGCGGCCCCGCAGCGCCCGCGCGCTC
CTCTCCCCGACTCGGAGCCCCTCGGCGGCGCCCGGCCCAGGACCC
GCCTAGGAGCGCAGGAGCCCCAGCGCAGAGACCCCAACGCCGAGA
CCCCCGCCCCGGCCCCGCCGCGCTTCCTCCCGACGCAGTTTAGGA
CCCTTGTTCGCGAAGAGGTGGTGTGCGGCTGAGACCCGCGTCCTC
AGGACGGTTCCATCAGTGCCTCGATCCTGCCCCACTGGAGGAGGA
AGGCAGCCCGAACAGCGCTCACCTAACTAACAGCTGCTGAGAGCT
GGGTTCCGTGGCCATGCACCTGGGACTGCCTTGAGAAGCGTGAGC
AAACCGCCCAGAGTAGAAGCGCTAGCCACCATGGATTGGGGCACG
CTGCAGACGATCCTGGGGGGTGTGAACAAACACTCCACCAGCATT
GGAAAGATCTGGCTCACCGTCCTCTTCATTTTTCGCATTATGATC
CTCGTTGTGGCTGCAAAGGAGGTGTGGGGAGATGAGCAGGCCGAC
TTTGTCTGCAACACCCTGCAGCCAGGCTGCAAGAACGTGTGCTAC
GATCACTACTTCCCCATCTCCCACATCCGGCTATGGGCCCTGCAG
CTGATCTTCGTGTCCACGCCAGCGCTCCTAGTGGCCATGCACGTG
GCCTACCGGAGACATGAGAAGAAGAGGAAGTTCATCAAGGGGGAG
ATAAAGAGTGAATTTAAGGACATCGAGGAGATCAAAACCCAGAAG
GTCCGCATCGAAGGCTCCCTGTGGTGGACCTACACAAGCAGCATC
TTCTTCCGGGTCATCTTCGAAGCCGCCTTCATGTACGTCTTCTAT
GTCATGTACGACGGCTTCTCCATGCAGCGGCTGGTGAAGTGCAAC
GCCTGGCCTTGTCCCAACACTGTGGACTGCTTTGTGTCCCGGCCC
ACGGAGAAGACTGTCTTCACAGTGTTCATGATTGCAGTGTCTGGA
ATTTGCATCCTGCTGAATGTCACTGAATTGTGTTATTTGCTAATT
AGATATTGTTCTGGGAAGTCAAAAAAGCCAGTTGGATCCCGGGCT
GACTACAAAGACCATGACGGTGATTATAAAGATCATGACATCGAC
TACAAGGATGACGATGACAAGTAAGAAATAGACAGCATGAGAGGG
ATGAGGCAACCCGTGCTCAGCTGTCAAGGCTCAGTCGCTAGCATT
TCCCAACACAAAGATTCTGACCTTAAATGCAACCATTTGAAACCC
CTGTAGGCCTCAGGTGAAACTCCAGATGCCACAATGGAGCTCTGC
TCCCCTAAAGCCTCAAAACAAAGGCCTAATTCTATGCCTGTCTTA
ATTTTCTTTCACTTAAGTTAGTTCCACTGAGACCCCAGGCTGTTA
GGGGTTATTGGTGTAAGGTACTTTCATATTTTAAACAGAGGATAT
CGGCATTTGTTTCTTTCTCTGAGGACAAGAGAAAAAAGCCAGGTT
CCACAGAGGACACAGAGAAGGTTTGGGTGTCCTCCTGGGGTTCTT
TTTGCCAACTTTCCCCACGTTAAAGGTGAACATTGGTTCTTTCAT
TTGCTTTGGAAGTTTTAATCTCTAACAGTGGACAAAGTTACCAGT
GCCTTAAACTCTGTTACACTTTTTGGAAGTGAAAACTTTGTAGTA
TGATAGGTTATTTTGATGTAAAGATGTTCTGGATACCATTATATG
TTCCCCCTGTTTCAGAGGCTCAGATTGTAATATGTAAATGGTATG
TCATTCGCTACTATGATTTAATTTGAAATATGGTCTTTTGGTTAT
GAATACTTTGCAGCACAGCTGAGAGGCTGTCTGTTGTATTCATTG
TGGTCATAGCACCTAACAACATTGTAGCCTCAATCGAGTGAGACA
GACTAGAAGTTCCTAGTGATGGCTTATGATAGCAAATGGCCTCAT
GTCAAATATTTAGATGTAATTTTGTGTAAGAAATACAGACTGGAT
GTACCACCAACTACTACCTGTAATGACAGGCCTGTCCAACACATC
TCCCTTTTCCATGACTGTGGTAGCCAGCATCGGAAAGAACGCTGA
TTTAAAGAGGTCGCTTGGGAATTTTATTGACACAGTACCATTTAA
TGGGGAGGACAAAATGGGGCAGGGGAGGGAGAAGTTTCTGTCGTT
AAAAACAGATTTGGAAAGACTGGACTCTAAAGTCTGTTGATTAAA
GATGAGCTTTGTCTACTTCAAAAGTTTGTTTGCTTACCCCTTCAG
CCTCCAATTTTTTAAGTGAAAATATAGCTAATAACATGTGAAAAG
AATAGAAGCTAAGGTTTAGATAAATATTGAGCAGATCTATAGGAA
GATTGAACCTGAATATTGCCATTATGCTTGACATGGTTTCCAAAA
AATGGTACTCCACATATTTCAGTGAGGGTAAGTATTTTCCTGTTG
TCAAGAATAGCATTGTAAAAGCATTTTGTAATAATAAAGAATAGC
TTTAATGATATGCTTGTAACTAAAATAATTTTGTAATGTATCAAA
TACATTTAAAACATTAAAATATAATCTCTATAATAAGAGCTCGCT
GATCAGCCTCGACTGTGCCTTCTAGTTGCCAGCCATCTGTTGTTT
GCCCCTCCCCCGTGCCTTCCTTGACCCTGGAAGGTGCCACTCCCA
CTGTCCTTTCCTAATAAAATGAGGAAATTGCATCGCATTGTCTGA
GTAGGTGTCATTCTATTCTGGGGGGTGGGGTGGGGCAGGACAGCA
AGGGGGAGGATTGGGAAGACAATAGCAGGCATGCTGGGGATGCGG
TGGGCTCTATGGAAGCTTGAATTCAGCTGACGTGCCTCGGACCGC
TAGGAACCCCTAGTGATGGAGTTGGCCACTCCCTCTCTGCGCGCT
CGCTCGCTCACTGAGGCCGGGCGACCAAAGGTCGCCCGACGCCCG
GGCTTTGCCCGGGCGGCCTCAGTGAGCGAGCGAGCGCGCAGCTGC
CTGCAGGGGCGCCTGATGCGGTATTTTCTCCTTACGCATCTGTGC
GGTATTTCACACCGCATACGTCAAAGCAACCATAGTACGCGCCCT
GTAGCGGCGCATTAAGCGCGGCGGGTGTGGTGGTTACGCGCAGCG
TGACCGCTACACTTGCCAGCGCCCTAGCGCCCGCTCCTTTCGCTT
TCTTCCCTTCCTTTCTCGCCACGTTCGCCGGCTTTCCCCGTCAAG
CTCTAAATCGGGGGCTCCCTTTAGGGTTCCGATTTAGTGCTTTAC
GGCACCTCGACCCCAAAAAACTTGATTTGGGTGATGGTTCACGTA
GTGGGCCATCGCCCTGATAGACGGTTTTTCGCCCTTTGACGTTGG
AGTCCACGTTCTTTAATAGTGGACTCTTGTTCCAAACTGGAACAA
CACTCAACCCTATCTCGGGCTATTCTTTTGATTTATAAGGGATTT
TGCCGATTTCGGCCTATTGGTTAAAAAATGAGCTGATTTAACAAA
AATTTAACGCGAATTTTAACAAAATATTAACGTTTACAATTTTAT
GGTGCACTCTCAGTACAATCTGCTCTGATGCCGCATAGTTAAGCC
AGCCCCGACACCCGCCAACACCCGCTGACGCGCCCTGACGGGCTT
GTCTGCTCCCGGCATCCGCTTACAGACAAGCTGTGACCGTCTCCG
GGAGCTGCATGTGTCAGAGGTTTTCACCGTCATCACCGAAACGCG
CGAGACGAAAGGGCCTCGTGATACGCCTATTTTTATAGGTTAATG
TCATGAACAATAAAACTGTCTGCTTACATAAACAGTAATACAAGG
GGTGTTATGAGCCATATTCAACGGGAAACGTCGAGGCCGCGATTA
AATTCCAACATGGATGCTGATTTATATGGGTATAAATGGGCTCGC
GATAATGTCGGGCAATCAGGTGCGACAATCTATCGCTTGTATGGG
AAGCCCGATGCGCCAGAGTTGTTTCTGAAACATGGCAAAGGTAGC
GTTGCCAATGATGTTACAGATGAGATGGTCAGACTAAACTGGCTG
ACGGAATTTATGCCTCTTCCGACCATCAAGCATTTTATCCGTACT
CCTGATGATGCATGGTTACTCACCACTGCGATCCCCGGAAAAACA
GCATTCCAGGTATTAGAAGAATATCCTGATTCAGGTGAAAATATT
GTTGATGCGCTGGCAGTGTTCCTGCGCCGGTTGCATTCGATTCCT
GTTTGTAATTGTCCTTTTAACAGCGATCGCGTATTTCGTCTCGCT
CAGGCGCAATCACGAATGAATAACGGTTTGGTTGATGCGAGTGAT
TTTGATGACGAGCGTAATGGCTGGCCTGTTGAACAAGTCTGGAAA
GAAATGCATAAACTTTTGCCATTCTCACCGGATTCAGTCGTCACT
CATGGTGATTTCTCACTTGATAACCTTATTTTTGACGAGGGGAAA
TTAATAGGTTGTATTGATGTTGGACGAGTCGGAATCGCAGACCGA
TACCAGGATCTTGCCATCCTATGGAACTGCCTCGGTGAGTTTTCT
CCTTCATTACAGAAACGGCTTTTTCAAAAATATGGTATTGATAAT
CCTGATATGAATAAATTGCAGTTTCATTTGATGCTCGATGAGTTT
TTCTAATCTCATGACCAAAATCCCTTAACGTGAGTTTTCGTTCCA
CTGAGCGTCAGACCCCGTAGAAAAGATCAAAGGATCTTCTTGAGA
TCCTTTTTTTCTGCGCGTAATCTGCTGCTTGCAAACAAAAAAACC
ACCGCTACCAGCGGTGGTTTGTTTGCCGGATCAAGAGCTACCAAC
TCTTTTTCCGAAGGTAACTGGCTTCAGCAGAGCGCAGATACCAAA
TACTGTCCTTCTAGTGTAGCCGTAGTTAGGCCACCACTTCAAGAA
CTCTGTAGCACCGCCTACATACCTCGCTCTGCTAATCCTGTTACC
AGTGGCTGCTGCCAGTGGCGATAAGTCGTGTCTTACCGGGTTGGA
CTCAAGACGATAGTTACCGGATAAGGCGCAGCGGTCGGGCTGAAC
GGGGGGTTCGTGCACACAGCCCAGCTTGGAGCGAACGACCTACAC
CGAACTGAGATACCTACAGCGTGAGCTATGAGAAAGCGCCACGCT
TCCCGAAGGGAGAAAGGCGGACAGGTATCCGGTAAGCGGCAGGGT
CGGAACAGGAGAGCGCACGAGGGAGCTTCCAGGGGGAAACGCCTG
GTATCTTTATAGTCCTGTCGGGTTTCGCCACCTCTGACTTGAGCG
TCGATTTTTGTGATGCTCGTCAGGGGGGCGGAGCCTATGGAAAAA
CGCCAGCAACGCGGCCTTTTTACGGTTCCTGGCCTTTTGCTGGCC
TTTTGCTCACATGT

TABLE 12
Components of Construct Sequence (SEQ ID NO: 103)

| Components | Position in construct |
|---|---|
| 5′ITR | 12-130 |
| Cloning site | 131-147 |
| KLHL14 promoter | 148-1402 |
| hGJB2 minimal promoter | 1403-1530 |
| Cloning site | 1531-1539 |
| Synthetic barcode | 1540-1547 |
| 5′UTR | 1548-1909 |
| GJB2 (exon2) | 1921-2598 |
| 3xFLAG | 2611-2676 |
| 3′UTR (exon2) | 2680-4086 |
| bGHpA | 4108-4332 |
| Cloning site | 4333-4366 |
| 3′ITR | 4367-4496 |

Exemplary Construct sequence
(SEQ ID NO: 104)
CCTGCAGGCAGCTGCGCGCTCGCTCGCTCACTGAGGCCGCCCGGG
CGTCGGGCGACCTTTGGTCGCCCGGCCTCAGTGAGCGAGCGAGCG
CGCAGAGAGGGAGTGGCCAACTCCATCACTAGGGGTTCCTGCGGC
CGCACGCGTGGTCCTTCCTCCTCCAGGGCCCTCTGCAGACCAGGC
TGAGATGGAGGAACCTGCTAAAATCGATGGAGATGCTTCTAGCCT
CCCAGTAGGAGGCCCCAGCCATGCCTTCAACCTGGCAGGAGGTGT
AGCCACTCCTCATCCTTGGGTTGCAGGTTGGGTGCTGCTGTTGTG
GTCCTTCCCAGAAACTGCCAGTAGAGGGCAGCCTGGGCATCCTAA
TGCTTACTCTGGTTGTTACACAAAGAAAATATTGGGGTCACTGGC
GAGCCCACCCACACTCACCAGAATCTCCACTGTAGTCCCCCTAAC
AAACAGCCCTTCACTTCCTCTCCCACTTCAGCAATTTGTATTTTG
ATGCCATTGGCCTCAGATCAGAGTGTTTTAAATCATCACGCCCTG
GCTTATCCCTGGTCGAGCCAGGACACGGGGTGCTTCAGTGGGTCT
GTCACCCTCTCTCCTTGAAGCATGTTGCTTTTATTTATTTACTTT
TACTCTCACCCTGCTCCTGTACCAGCAGGGGCCACTTCAAAGCCA
AGGTACAGGGTGATAACTTGTGGTCCAGCATCAGTTTTCTCCACT
TCTTTCTCCCACTCACCCCCAGCAAGGTGCCTGGGGAGACTTGAG
CAGATGTTTCATTTTGGCCTGGCCAGTGGCTGAAAGCCAGGCCTC
CAATGCACTGTGACCTCTGGCTTCCCCAGCAGCTTTCCCAGAGAG
GCAGAGGGAGTCTTCATTCTTCCCAGGCGGGGAGACCACGCCTTC
CCTGCCTCCTCCCTCCGCGGGGGGTCGCGTTGGAGGTCACCCCCG
CCCCCTAGGCGCTGGGTTGGGAGTGACGCGGGGTGGGCTGGAGAG
GTTTCCTGCCGTCTGGGAAGCGTAAACGGACCGCCCACCTGTCGG
GCCTCGGCCGCCCGCACCTGCTTGTGAGAAGCCTGCGGCTGGGGC
ACCGCCCCCGGTCCCCGCCCGGGTCCGCGCATTGGGAGCACACTG
GCCCTTTAAGAGCGCGGCGGCCGCGGCGCGCGGGAAGCTCTGAGG
ACCCAGAGGCCGGGCGCGCTCCGCCCGCGGCGCCGCCCCCTCCGT
AACTTTCCCAGTCTCCGAGGGAAGAGGCGGGGTGTGGGGTGCGGT
TAAAAGGCGCCACGGCGGGAGACAGGTCTCACCGGTTTCACTGGG
TTGCGGCCCCGCAGCGCCCGCGCGCTCCTCTCCCCGACTCGGAGC
CCCTCGGCGGCGCCCGGCCCAGGACCCGCCTAGGAGCGCAGGAGC
CCCAGCGCAGAGACCCCAACGCCGAGACCCCCGCCCCGGCCCCGC
CGCGCTTCCTCCCGACGCAGTTTAGGACCCTTGTTCGCGAAGAGG
TGGTGTGCGGCTGAGACCCGCGTCCTCAGGACGGTTCCATCAGTG
CCTCGATCCTGCCCCACTGGAGGAGGAAGGCAGCCCGAACAGCGC
TCACCTAACTAACAGCTGCTGAGAGCTGGGTTCCGTGGCCATGCA
CCTGGGACTGCCTTGAGAAGCGTGAGCAAACCGCCCAGAGTAGAA
GCGCTAGCCACCATGGATTGGGGCACGCTGCAGACGATCCTGGGG
GGTGTGAACAAACACTCCACCAGCATTGGAAAGATCTGGCTCACC
GTCCTCTTCATTTTTCGCATTATGATCCTCGTTGTGGCTGCAAAG
GAGGTGTGGGGAGATGAGCAGGCCGACTTTGTCTGCAACACCCTG
CAGCCAGGCTGCAAGAACGTGTGCTACGATCACTACTTCCCCATC
TCCCACATCCGGCTATGGGCCCTGCAGCTGATCTTCGTGTCCACG
CCAGCGCTCCTAGTGGCCATGCACGTGGCCTACCGGAGACATGAG
AAGAAGAGGAAGTTCATCAAGGGGGAGATAAAGAGTGAATTTAAG
GACATCGAGGAGATCAAAACCCAGAAGGTCCGCATCGAAGGCTCC
CTGTGGTGGACCTACACAAGCAGCATCTTCTTCCGGGTCATCTTC
GAAGCCGCCTTCATGTACGTCTTCTATGTCATGTACGACGGCTTC
TCCATGCAGCGGCTGGTGAAGTGCAACGCCTGGCCTTGTCCCAAC
ACTGTGGACTGCTTTGTGTCCCGGCCCACGGAGAAGACTGTCTTC
ACAGTGTTCATGATTGCAGTGTCTGGAATTTGCATCCTGCTGAAT
GTCACTGAATTGTGTTATTTGCTAATTAGATATTGTTCTGGGAAG
TCAAAAAAGCCAGTTGGATCCCGGGCTGACTACAAAGACCATGAC
GGTGATTATAAAGATCATGACATCGACTACAAGGATGACGATGAC
AAGTAAGAAATAGACAGCATGAGAGGGATGAGGCAACCCGTGCTC
AGCTGTCAAGGCTCAGTCGCTAGCATTTCCCAACACAAAGATTCT
GACCTTAAATGCAACCATTTGAAACCCCTGTAGGCCTCAGGTGAA
ACTCCAGATGCCACAATGGAGCTCTGCTCCCCTAAAGCCTCAAAA
CAAAGGCCTAATTCTATGCCTGTCTTAATTTTCTTTCACTTAAGT
TAGTTCCACTGAGACCCCAGGCTGTTAGGGGTTATTGGTGTAAGG
TACTTTCATATTTTAAACAGAGGATATCGGCATTTGTTTCTTTCT
CTGAGGACAAGAGAAAAAAGCCAGGTTCCACAGAGGACACAGAGA
AGGTTTGGGTGTCCTCCTGGGGTTCTTTTTGCCAACTTTCCCCAC
GTTAAAGGTGAACATTGGTTCTTTCATTTGCTTTGGAAGTTTTAA
TCTCTAACAGTGGACAAAGTTACCAGTGCCTTAAACTCTGTTACA
CTTTTTGGAAGTGAAAACTTTGTAGTATGATAGGTTATTTTGATG
TAAAGATGTTCTGGATACCATTATATGTTCCCCCTGTTTCAGAGG
CTCAGATTGTAATATGTAAATGGTATGTCATTCGCTACTATGATT
TAATTTGAAATATGGTCTTTTGGTTATGAATACTTTGCAGCACAG
CTGAGAGGCTGTCTGTTGTATTCATTGTGGTCATAGCACCTAACA
ACATTGTAGCCTCAATCGAGTGAGACAGACTAGAAGTTCCTAGTG
ATGGCTTATGATAGCAAATGGCCTCATGTCAAATATTTAGATGTA
ATTTTGTGTAAGAAATACAGACTGGATGTACCACCAACTACTACC
TGTAATGACAGGCCTGTCCAACACATCTCCCTTTTCCATGACTGT
GGTAGCCAGCATCGGAAAGAACGCTGATTTAAAGAGGTCGCTTGG
GAATTTTATTGACACAGTACCATTTAATGGGGAGGACAAAATGGG
GCAGGGGAGGGAGAAGTTTCTGTCGTTAAAAACAGATTTGGAAAG
ACTGGACTCTAAAGTCTGTTGATTAAAGATGAGCTTTGTCTACTT
CAAAAGTTTGTTTGCTTACCCCTTCAGCCTCCAATTTTTTAAGTG
AAAATATAGCTAATAACATGTGAAAAGAATAGAAGCTAAGGTTTA
GATAAATATTGAGCAGATCTATAGGAAGATTGAACCTGAATATTG
CCATTATGCTTGACATGGTTTCCAAAAAATGGTACTCCACATATT
TCAGTGAGGGTAAGTATTTTCCTGTTGTCAAGAATAGCATTGTAA
AAGCATTTTGTAATAATAAAGAATAGCTTTAATGATATGCTTGTA
ACTAAAATAATTTTGTAATGTATCAAATACATTTAAAACATTAAA
ATATAATCTCTATAATAAGAGCTCGCTGATCAGCCTCGACTGTGC
CTTCTAGTTGCCAGCCATCTGTTGTTTGCCCCTCCCCCGTGCCTT
CCTTGACCCTGGAAGGTGCCACTCCCACTGTCCTTTCCTAATAAA
ATGAGGAAATTGCATCGCATTGTCTGAGTAGGTGTCATTCTATTC
TGGGGGGTGGGGTGGGGCAGGACAGCAAGGGGGAGGATTGGGAAG
ACAATAGCAGGCATGCTGGGGATGCGGTGGGCTCTATGGAAGCTT
GAATTCAGCTGACGTGCCTCGGACCGCTAGGAACCCCTAGTGATG
GAGTTGGCCACTCCCTCTCTGCGCGCTCGCTCGCTCACTGAGGCC
GGGCGACCAAAGGTCGCCCGACGCCCGGGCTTTGCCCGGGCGGCC
TCAGTGAGCGAGCGAGCGCGCAGCTGCCTGCAGGGGCGCCTGATG
CGGTATTTTCTCCTTACGCATCTGTGCGGTATTTCACACCGCATA
CGTCAAAGCAACCATAGTACGCGCCCTGTAGCGGCGCATTAAGCG
CGGCGGGTGTGGTGGTTACGCGCAGCGTGACCGCTACACTTGCCA
GCGCCCTAGCGCCCGCTCCTTTCGCTTTCTTCCCTTCCTTTCTCG
CCACGTTCGCCGGCTTTCCCCGTCAAGCTCTAAATCGGGGGCTCC
CTTTAGGGTTCCGATTTAGTGCTTTACGGCACCTCGACCCCAAAA
AACTTGATTTGGGTGATGGTTCACGTAGTGGGCCATCGCCCTGAT
AGACGGTTTTTCGCCCTTTGACGTTGGAGTCCACGTTCTTTAATA
GTGGACTCTTGTTCCAAACTGGAACAACACTCAACCCTATCTCGG
GCTATTCTTTTGATTTATAAGGGATTTTGCCGATTTCGGCCTATT
GGTTAAAAAATGAGCTGATTTAACAAAAATTTAACGCGAATTTTA
ACAAAATATTAACGTTTACAATTTTATGGTGCACTCTCAGTACAA
TCTGCTCTGATGCCGCATAGTTAAGCCAGCCCCGACACCCGCCAA
CACCCGCTGACGCGCCCTGACGGGCTTGTCTGCTCCCGGCATCCG
CTTACAGACAAGCTGTGACCGTCTCCGGGAGCTGCATGTGTCAGA
GGTTTTCACCGTCATCACCGAAACGCGCGAGACGAAAGGGCCTCG
TGATACGCCTATTTTTATAGGTTAATGTCATGAACAATAAAACTG
TCTGCTTACATAAACAGTAATACAAGGGGTGTTATGAGCCATATT
CAACGGGAAACGTCGAGGCCGCGATTAAATTCCAACATGGATGCT
GATTTATATGGGTATAAATGGGCTCGCGATAATGTCGGGCAATCA
GGTGCGACAATCTATCGCTTGTATGGGAAGCCCGATGCGCCAGAG
TTGTTTCTGAAACATGGCAAAGGTAGCGTTGCCAATGATGTTACA
GATGAGATGGTCAGACTAAACTGGCTGACGGAATTTATGCCTCTT
CCGACCATCAAGCATTTTATCCGTACTCCTGATGATGCATGGTTA
CTCACCACTGCGATCCCCGGAAAAACAGCATTCCAGGTATTAGAA
GAATATCCTGATTCAGGTGAAAATATTGTTGATGCGCTGGCAGTG
TTCCTGCGCCGGTTGCATTCGATTCCTGTTTGTAATTGTCCTTTT
AACAGCGATCGCGTATTTCGTCTCGCTCAGGCGCAATCACGAATG
AATAACGGTTTGGTTGATGCGAGTGATTTTGATGACGAGCGTAAT
GGCTGGCCTGTTGAACAAGTCTGGAAAGAAATGCATAAACTTTTG
CCATTCTCACCGGATTCAGTCGTCACTCATGGTGATTTCTCACTT
GATAACCTTATTTTTGACGAGGGGAAATTAATAGGTTGTATTGAT
GTTGGACGAGTCGGAATCGCAGACCGATACCAGGATCTTGCCATC
CTATGGAACTGCCTCGGTGAGTTTTCTCCTTCATTACAGAAACGG
CTTTTTCAAAAATATGGTATTGATAATCCTGATATGAATAAATTG
CAGTTTCATTTGATGCTCGATGAGTTTTTCTAATCTCATGACCAA
AATCCCTTAACGTGAGTTTTCGTTCCACTGAGCGTCAGACCCCGT
AGAAAAGATCAAAGGATCTTCTTGAGATCCTTTTTTTCTGCGCGT
AATCTGCTGCTTGCAAACAAAAAAACCACCGCTACCAGCGGTGGT
TTGTTTGCCGGATCAAGAGCTACCAACTCTTTTTCCGAAGGTAAC
TGGCTTCAGCAGAGCGCAGATACCAAATACTGTCCTTCTAGTGTA
GCCGTAGTTAGGCCACCACTTCAAGAACTCTGTAGCACCGCCTAC
ATACCTCGCTCTGCTAATCCTGTTACCAGTGGCTGCTGCCAGTGG
CGATAAGTCGTGTCTTACCGGGTTGGACTCAAGACGATAGTTACC
GGATAAGGCGCAGCGGTCGGGCTGAACGGGGGGTTCGTGCACACA
GCCCAGCTTGGAGCGAACGACCTACACCGAACTGAGATACCTACA
GCGTGAGCTATGAGAAAGCGCCACGCTTCCCGAAGGGAGAAAGGC
GGACAGGTATCCGGTAAGCGGCAGGGTCGGAACAGGAGAGCGCAC
GAGGGAGCTTCCAGGGGGAAACGCCTGGTATCTTTATAGTCCTGT
CGGGTTTCGCCACCTCTGACTTGAGCGTCGATTTTTGTGATGCTC
GTCAGGGGGGCGGAGCCTATGGAAAAACGCCAGCAACGCGGCCTT
TTTACGGTTCCTGGCCTTTTGCTGGCCTTTTGCTCACATG
T

TABLE 13
Components of Construct Sequence (SEQ ID NO: 104)

| Components | Position in construct |
|---|---|
| 5′ITR | 12-130 |
| Cloning site | 131-147 |
| MMP15 promoter | 148-1159 |
| hGJB2 minimal promoter | 1160-1287 |
| Cloning site | 1288-1296 |
| Synthetic barcode | 1297-1304 |
| 5′UTR | 1305-1666 |
| GJB2 (exon2) | 1678-2355 |
| 3xFLAG | 2368-2433 |
| 3′UTR (exon2) | 2437-3843 |
| bGHpA | 3865-4089 |
| Cloning site | 4090-4123 |
| 3′ITR | 4124-4253 |

Exemplary Construct sequence
(SEQ ID NO: 105)
CCTGCAGGCAGCTGCGCGCTCGCTCGCTCACTGAGGCCGCCCGGG
CGTCGGGCGACCTTTGGTCGCCCGGCCTCAGTGAGCGAGCGAGCG
CGCAGAGAGGGAGTGGCCAACTCCATCACTAGGGGTTCCTGCGGC
CGCACGCGTGGTCAGGCTACCTCTCAGGCTGACTGAGTCATGCAG
CATAGGCTGCCACGTCTCTGGGCTGGCGGGGCCGTCATTATTCCT
GGCCTCACTGCAGCTAAATTGAAGAAACGTTTGGTTTGTGGGCCA
CGTCAAGGAATGTGTAAGAGCTGCCACGTTGTCGGGTCTGGGTTA
TTGGGCTTTTCCCCTCCTTCAGAGAAGATTTCCAGGCGTGTGGGT
GGGGTTTCAGAAGAAAATTGATGCCTGCGTGTGAGTGTTCCCTGG
ACCTGGACCAGCAGCGGCAATATTACAGACCCGGGGGTTGGGGCA
GACTGAGCCAATCTCTGCACCGTCAAAGTTATGGATACAGAGCCC
TGGAAAAAGGCTGAAGGATAAGATAGCTGACATTTATGAAGTGCT
TCATTCATGTAGCAGTGGGCCAAATGCGTACTTTACACTTGAGGA
AGCTGAGGCTGGAGGTTGATAACATGCCTCAAGTCTTCTAGAGTT
AAATAACTTTGACCCAGGACCCAAGCCCAGAGTTCTGACTCAAAA
ACTAGGCCTCCTAAACATCCTCTTATATGAGGTTAAATTTCATCT
TCCTCTGTTTGGCCTTGGCCTGGTTGGTGGATGCTCTGCTTCGGG
GACCCAGGGCCAGATGACAATGGGTTCTTTGTGCCCTTCAGACAA
TGGGAAGGGCTGCCTGGGGAAAGATACAGTAACAAGGCAACAGGC
TGAGTCAGCCTCCAATGTGCTTGAACCTTCTTAGCTTGGCAGCCT
TGACATTCAGCCAGCCACACAAAGGGTATATCAAGGATGATACCA
CTAGTAGCAGCTTGTCTTGTCTGTACCTCTGAACAAGAAAGAGGC
TGTTCTGGGTCATCCCTCCAGGCCTGTCCAGCCCTGGCACTCTGT
GAGTCGGTTTAGGCAGCAGCCCCGGAACAGATGAGGCAGGCAGGG
TTGGGACGTTTGGTCAGGACAGCCCACCAGGAGGAAAGAAATGAA
AGACAGAGACAGCTTTGGCTATGGGAGAAGGAGGAGGCCGGGGGA
AGGAGGAGACAGGAGGAGGAGGGACCACGGGGTGGAGGGGAGATA
GACCCAGCCCAAAGCTCTGAGGACCCAGAGGCCGGGCGCGCTCCG
CCCGCGGCGCCGCCCCCTCCGTAACTTTCCCAGTCTCCGAGGGAA
GAGGCGGGGTGTGGGGTGCGGTTAAAAGGCGCCACGGCGGGAGAC
AGGTCTCACCGGTATACTCTCGTTGCGGCCCCGCAGCGCCCGCGC
GCTCCTCTCCCCGACTCGGAGCCCCTCGGCGGCGCCCGGCCCAGG
ACCCGCCTAGGAGCGCAGGAGCCCCAGCGCAGAGACCCCAACGCC
GAGACCCCCGCCCCGGCCCCGCCGCGCTTCCTCCCGACGCAGTTT
AGGACCCTTGTTCGCGAAGAGGTGGTGTGCGGCTGAGACCCGCGT
CCTCAGGACGGTTCCATCAGTGCCTCGATCCTGCCCCACTGGAGG
AGGAAGGCAGCCCGAACAGCGCTCACCTAACTAACAGCTGCTGAG
AGCTGGGTTCCGTGGCCATGCACCTGGGACTGCCTTGAGAAGCGT
GAGCAAACCGCCCAGAGTAGAAGCGCTAGCCACCATGGATTGGGG
CACGCTGCAGACGATCCTGGGGGGTGTGAACAAACACTCCACCAG
CATTGGAAAGATCTGGCTCACCGTCCTCTTCATTTTTCGCATTAT
GATCCTCGTTGTGGCTGCAAAGGAGGTGTGGGGAGATGAGCAGGC
CGACTTTGTCTGCAACACCCTGCAGCCAGGCTGCAAGAACGTGTG
CTACGATCACTACTTCCCCATCTCCCACATCCGGCTATGGGCCCT
GCAGCTGATCTTCGTGTCCACGCCAGCGCTCCTAGTGGCCATGCA
CGTGGCCTACCGGAGACATGAGAAGAAGAGGAAGTTCATCAAGGG
GGAGATAAAGAGTGAATTTAAGGACATCGAGGAGATCAAAACCCA
GAAGGTCCGCATCGAAGGCTCCCTGTGGTGGACCTACACAAGCAG
CATCTTCTTCCGGGTCATCTTCGAAGCCGCCTTCATGTACGTCTT
CTATGTCATGTACGACGGCTTCTCCATGCAGCGGCTGGTGAAGTG
CAACGCCTGGCCTTGTCCCAACACTGTGGACTGCTTTGTGTCCCG
GCCCACGGAGAAGACTGTCTTCACAGTGTTCATGATTGCAGTGTC
TGGAATTTGCATCCTGCTGAATGTCACTGAATTGTGTTATTTGCT
AATTAGATATTGTTCTGGGAAGTCAAAAAAGCCAGTTGGATCCCG
GGCTGACTACAAAGACCATGACGGTGATTATAAAGATCATGACAT
CGACTACAAGGATGACGATGACAAGTAAGAAATAGACAGCATGAG
AGGGATGAGGCAACCCGTGCTCAGCTGTCAAGGCTCAGTCGCTAG
CATTTCCCAACACAAAGATTCTGACCTTAAATGCAACCATTTGAA
ACCCCTGTAGGCCTCAGGTGAAACTCCAGATGCCACAATGGAGCT
CTGCTCCCCTAAAGCCTCAAAACAAAGGCCTAATTCTATGCCTGT
CTTAATTTTCTTTCACTTAAGTTAGTTCCACTGAGACCCCAGGCT
GTTAGGGGTTATTGGTGTAAGGTACTTTCATATTTTAAACAGAGG
ATATCGGCATTTGTTTCTTTCTCTGAGGACAAGAGAAAAAAGCCA
GGTTCCACAGAGGACACAGAGAAGGTTTGGGTGTCCTCCTGGGGT
TCTTTTTGCCAACTTTCCCCACGTTAAAGGTGAACATTGGTTCTT
TCATTTGCTTTGGAAGTTTTAATCTCTAACAGTGGACAAAGTTAC
CAGTGCCTTAAACTCTGTTACACTTTTTGGAAGTGAAAACTTTGT
AGTATGATAGGTTATTTTGATGTAAAGATGTTCTGGATACCATTA
TATGTTCCCCCTGTTTCAGAGGCTCAGATTGTAATATGTAAATGG
TATGTCATTCGCTACTATGATTTAATTTGAAATATGGTCTTTTGG
TTATGAATACTTTGCAGCACAGCTGAGAGGCTGTCTGTTGTATTC
ATTGTGGTCATAGCACCTAACAACATTGTAGCCTCAATCGAGTGA
GACAGACTAGAAGTTCCTAGTGATGGCTTATGATAGCAAATGGCC
TCATGTCAAATATTTAGATGTAATTTTGTGTAAGAAATACAGACT
GGATGTACCACCAACTACTACCTGTAATGACAGGCCTGTCCAACA
CATCTCCCTTTTCCATGACTGTGGTAGCCAGCATCGGAAAGAACG
CTGATTTAAAGAGGTCGCTTGGGAATTTTATTGACACAGTACCAT
TTAATGGGGAGGACAAAATGGGGCAGGGGAGGGAGAAGTTTCTGT
CGTTAAAAACAGATTTGGAAAGACTGGACTCTAAAGTCTGTTGAT
TAAAGATGAGCTTTGTCTACTTCAAAAGTTTGTTTGCTTACCCCT
TCAGCCTCCAATTTTTTAAGTGAAAATATAGCTAATAACATGTGA
AAAGAATAGAAGCTAAGGTTTAGATAAATATTGAGCAGATCTATA
GGAAGATTGAACCTGAATATTGCCATTATGCTTGACATGGTTTCC
AAAAAATGGTACTCCACATATTTCAGTGAGGGTAAGTATTTTCCT
GTTGTCAAGAATAGCATTGTAAAAGCATTTTGTAATAATAAAGAA
TAGCTTTAATGATATGCTTGTAACTAAAATAATTTTGTAATGTAT
CAAATACATTTAAAACATTAAAATATAATCTCTATAATAAGAGCT
CGCTGATCAGCCTCGACTGTGCCTTCTAGTTGCCAGCCATCTGTT
GTTTGCCCCTCCCCCGTGCCTTCCTTGACCCTGGAAGGTGCCACT
CCCACTGTCCTTTCCTAATAAAATGAGGAAATTGCATCGCATTGT
CTGAGTAGGTGTCATTCTATTCTGGGGGGGGGGTGGGGCAGGACA
GCAAGGGGGAGGATTGGGAAGACAATAGCAGGCATGCTGGGGATG
CGGTGGGCTCTATGGAAGCTTGAATTCAGCTGACGTGCCTCGGAC
CGCTAGGAACCCCTAGTGATGGAGTTGGCCACTCCCTCTCTGCGC
GCTCGCTCGCTCACTGAGGCCGGGCGACCAAAGGTCGCCCGACGC
CCGGGCTTTGCCCGGGCGGCCTCAGTGAGCGAGCGAGCGCGCAGC
TGCCTGCAGGGGCGCCTGATGCGGTATTTTCTCCTTACGCATCTG
TGCGGTATTTCACACCGCATACGTCAAAGCAACCATAGTACGCGC
CCTGTAGCGGCGCATTAAGCGCGGCGGGTGTGGTGGTTACGCGCA
GCGTGACCGCTACACTTGCCAGCGCCCTAGCGCCCGCTCCTTTCG
CTTTCTTCCCTTCCTTTCTCGCCACGTTCGCCGGCTTTCCCCGTC
AAGCTCTAAATCGGGGGCTCCCTTTAGGGTTCCGATTTAGTGCTT
TACGGCACCTCGACCCCAAAAAACTTGATTTGGGTGATGGTTCAC
GTAGTGGGCCATCGCCCTGATAGACGGTTTTTCGCCCTTTGACGT
TGGAGTCCACGTTCTTTAATAGTGGACTCTTGTTCCAAACTGGAA
CAACACTCAACCCTATCTCGGGCTATTCTTTTGATTTATAAGGGA
TTTTGCCGATTTCGGCCTATTGGTTAAAAAATGAGCTGATTTAAC
AAAAATTTAACGCGAATTTTAACAAAATATTAACGTTTACAATTT
TATGGTGCACTCTCAGTACAATCTGCTCTGATGCCGCATAGTTAA
GCCAGCCCCGACACCCGCCAACACCCGCTGACGCGCCCTGACGGG
CTTGTCTGCTCCCGGCATCCGCTTACAGACAAGCTGTGACCGTCT
CCGGGAGCTGCATGTGTCAGAGGTTTTCACCGTCATCACCGAAAC
GCGCGAGACGAAAGGGCCTCGTGATACGCCTATTTTTATAGGTTA
ATGTCATGAACAATAAAACTGTCTGCTTACATAAACAGTAATACA
AGGGGTGTTATGAGCCATATTCAACGGGAAACGTCGAGGCCGCGA
TTAAATTCCAACATGGATGCTGATTTATATGGGTATAAATGGGCT
CGCGATAATGTCGGGCAATCAGGTGCGACAATCTATCGCTTGTAT
GGGAAGCCCGATGCGCCAGAGTTGTTTCTGAAACATGGCAAAGGT
AGCGTTGCCAATGATGTTACAGATGAGATGGTCAGACTAAACTGG
CTGACGGAATTTATGCCTCTTCCGACCATCAAGCATTTTATCCGT
ACTCCTGATGATGCATGGTTACTCACCACTGCGATCCCCGGAAAA
ACAGCATTCCAGGTATTAGAAGAATATCCTGATTCAGGTGAAAAT
ATTGTTGATGCGCTGGCAGTGTTCCTGCGCCGGTTGCATTCGATT
CCTGTTTGTAATTGTCCTTTTAACAGCGATCGCGTATTTCGTCTC
GCTCAGGCGCAATCACGAATGAATAACGGTTTGGTTGATGCGAGT
GATTTTGATGACGAGCGTAATGGCTGGCCTGTTGAACAAGTCTGG
AAAGAAATGCATAAACTTTTGCCATTCTCACCGGATTCAGTCGTC
ACTCATGGTGATTTCTCACTTGATAACCTTATTTTTGACGAGGGG
AAATTAATAGGTTGTATTGATGTTGGACGAGTCGGAATCGCAGAC
CGATACCAGGATCTTGCCATCCTATGGAACTGCCTCGGTGAGTTT
TCTCCTTCATTACAGAAACGGCTTTTTCAAAAATATGGTATTGAT
AATCCTGATATGAATAAATTGCAGTTTCATTTGATGCTCGATGAG
TTTTTCTAATCTCATGACCAAAATCCCTTAACGTGAGTTTTCGTT
CCACTGAGCGTCAGACCCCGTAGAAAAGATCAAAGGATCTTCTTG
AGATCCTTTTTTTCTGCGCGTAATCTGCTGCTTGCAAACAAAAAA
ACCACCGCTACCAGCGGTGGTTTGTTTGCCGGATCAAGAGCTACC
AACTCTTTTTCCGAAGGTAACTGGCTTCAGCAGAGCGCAGATACC
AAATACTGTCCTTCTAGTGTAGCCGTAGTTAGGCCACCACTTCAA
GAACTCTGTAGCACCGCCTACATACCTCGCTCTGCTAATCCTGTT
ACCAGTGGCTGCTGCCAGTGGCGATAAGTCGTGTCTTACCGGGTT
GGACTCAAGACGATAGTTACCGGATAAGGCGCAGCGGTCGGGCTG
AACGGGGGGTTCGTGCACACAGCCCAGCTTGGAGCGAACGACCTA
CACCGAACTGAGATACCTACAGCGTGAGCTATGAGAAAGCGCCAC
GCTTCCCGAAGGGAGAAAGGCGGACAGGTATCCGGTAAGCGGCAG
GGTCGGAACAGGAGAGCGCACGAGGGAGCTTCCAGGGGGAAACGC
CTGGTATCTTTATAGTCCTGTCGGGTTTCGCCACCTCTGACTTGA
GCGTCGATTTTTGTGATGCTCGTCAGGGGGGCGGAGCCTATGGAA
AAACGCCAGCAACGCGGCCTTTTTACGGTTCCTGGCCTTTTGCTG
GCCTTTTGCTCACATGT

TABLE 14
Components of Construct Sequence (SEQ ID NO: 105)

| Components | Position in construct |
|---|---|
| 5′ITR | 12-130 |
| Cloning site | 131-147 |
| SPARC promoter | 148-1226 |
| hGJB2 minimal promoter | 1227-1354 |
| Cloning site | 1355-1363 |
| Synthetic barcode | 1364-1371 |
| 5′UTR | 1372-1733 |
| GJB2 (exon2) | 1745-2422 |
| 3xFLAG | 2435-2500 |
| 3′UTR (exon2) | 2504-3910 |
| bGHpA | 3932-4156 |
| Cloning site | 4157-4190 |
| 3′ITR | 4191-4320 |

Exemplary Construct sequence
(SEQ ID NO: 106)
CCTGCAGGCAGCTGCGCGCTCGCTCGCTCACTGAGGCCGCCCGGG
CGTCGGGCGACCTTTGGTCGCCCGGCCTCAGTGAGCGAGCGAGCG
CGCAGAGAGGGAGTGGCCAACTCCATCACTAGGGGTTCCTGCGGC
CGCACGCGTGGTCCAAGGACTCTTTTTTCTAAACTTCCCTTCATC
TTCTAGTTTGACGCCCTTGGTGGGAAAAGTGTCTGAGATAAGGAA
AAGGCATCCTTTCAGTTCTCTGATACTATCTTGAAGCGAGGGATG
GAGAAAGGCAAAGAGAGACACAGGAGAAGCGTATCCCCTGGGAAC
AGGTGTCTAGTGGAGTCCAGTAACTCACAGTCTCTCAGTTCCGTC
AGCACTGTCCCTTGGGTCGCAAATTTCTTCCATTAGCCCTTCCAC
CAGCTGTATTTCAAATGGGGCTGGACAATAATTGTGGCCAGTGGC
CTTGTGTTGTTTGTACTTGCGGACTAGTAGTTCTCACCTGTCTTT
CTCTGACTCCTATTAGCCACTGGGATTTCAGCAGCTGGTTCAGCC
AATTCTACTCAATTCAACATTAAGTTGCAGTGGGCTAGAACTCAT
GGGCCGATTTAACAAGTGAAATTCTACCAAGATACATCAAAAATA
GCAACAGGACTAGATACTCAGCTCATTTTGTTTTATTTGTAATAT
ACCAGTTGTGGCTTTAGTGCCAGTCTGATTCATCTCTCTACTACA
AAATGAGGCTCTATAAAGGAAAATATTGCAACTGGAGTGAGGAAT
TTGAATCTTATAGGAAGGAATTTGTCTTCTCATGAAGACTTCAGT
TTACCAGAAGTATCTATTGAGGAAGTGTTTACAAGAAAATGTGCC
ATTTAGCTTTATTCTAAATTTGCATAATAACTGAACCAAACAAAA
AAATATAGATAGATAGATTGTTCTATCTATAGATAGATAGGGAAC
ATTGGCAGTAGGTGGCAGTAAGTTCCCCTGAGCACATGGAGGACA
CAGTTAAATGCATTTGAGGTATGTGGGAAATGGTTTAAAGCAGAA
TTTTATGCCAACTTTTAGTAACGGAAGCCTAACAAATGTTTGTTC
TTTCAAGTGAGAGAAGCAAGCAATCTGGAACTATTCATAAGCTTA
TTTTCTGTATCCTTAAACATATTTTATAATGAATGTATGATTTAA
ATAGTAAGTTAAGTGTCTGGGGGTACTGCACACCTCCCTTGCATA
CAGTCAAACTTCTTCAGGGTGATGGGGAAGAGGAGTTATAGGCTG
CCAAGCAAAATTGCCAAACTGGTCTCAGAAATTCACTGCATTGGA
GAGCGCGGGATCCTTGCAACACTGACTTTAGCAGTTAAACTAGAG
TGGTTGGGGATGAGTATTCTAAGCTCTGAGGACCCAGAGGCCGGG
CGCGCTCCGCCCGCGGCGCCGCCCCCTCCGTAACTTTCCCAGTCT
CCGAGGGAAGAGGCGGGGTGTGGGGTGCGGTTAAAAGGCGCCACG
GCGGGAGACAGGTCTCACCGGTGGCACTTCGTTGCGGCCCCGCAG
CGCCCGCGCGCTCCTCTCCCCGACTCGGAGCCCCTCGGCGGCGCC
CGGCCCAGGACCCGCCTAGGAGCGCAGGAGCCCCAGCGCAGAGAC
CCCAACGCCGAGACCCCCGCCCCGGCCCCGCCGCGCTTCCTCCCG
ACGCAGTTTAGGACCCTTGTTCGCGAAGAGGTGGTGTGCGGCTGA
GACCCGCGTCCTCAGGACGGTTCCATCAGTGCCTCGATCCTGCCC
CACTGGAGGAGGAAGGCAGCCCGAACAGCGCTCACCTAACTAACA
GCTGCTGAGAGCTGGGTTCCGTGGCCATGCACCTGGGACTGCCTT
GAGAAGCGTGAGCAAACCGCCCAGAGTAGAAGCGCTAGCCACCAT
GGATTGGGGCACGCTGCAGACGATCCTGGGGGGTGTGAACAAACA
CTCCACCAGCATTGGAAAGATCTGGCTCACCGTCCTCTTCATTTT
TCGCATTATGATCCTCGTTGTGGCTGCAAAGGAGGTGTGGGGAGA
TGAGCAGGCCGACTTTGTCTGCAACACCCTGCAGCCAGGCTGCAA
GAACGTGTGCTACGATCACTACTTCCCCATCTCCCACATCCGGCT
ATGGGCCCTGCAGCTGATCTTCGTGTCCACGCCAGCGCTCCTAGT
GGCCATGCACGTGGCCTACCGGAGACATGAGAAGAAGAGGAAGTT
CATCAAGGGGGAGATAAAGAGTGAATTTAAGGACATCGAGGAGAT
CAAAACCCAGAAGGTCCGCATCGAAGGCTCCCTGTGGTGGACCTA
CACAAGCAGCATCTTCTTCCGGGTCATCTTCGAAGCCGCCTTCAT
GTACGTCTTCTATGTCATGTACGACGGCTTCTCCATGCAGCGGCT
GGTGAAGTGCAACGCCTGGCCTTGTCCCAACACTGTGGACTGCTT
TGTGTCCCGGCCCACGGAGAAGACTGTCTTCACAGTGTTCATGAT
TGCAGTGTCTGGAATTTGCATCCTGCTGAATGTCACTGAATTGTG
TTATTTGCTAATTAGATATTGTTCTGGGAAGTCAAAAAAGCCAGT
TGGATCCCGGGCTGACTACAAAGACCATGACGGTGATTATAAAGA
TCATGACATCGACTACAAGGATGACGATGACAAGTAAGAAATAGA
CAGCATGAGAGGGATGAGGCAACCCGTGCTCAGCTGTCAAGGCTC
AGTCGCTAGCATTTCCCAACACAAAGATTCTGACCTTAAATGCAA
CCATTTGAAACCCCTGTAGGCCTCAGGTGAAACTCCAGATGCCAC
AATGGAGCTCTGCTCCCCTAAAGCCTCAAAACAAAGGCCTAATTC
TATGCCTGTCTTAATTTTCTTTCACTTAAGTTAGTTCCACTGAGA
CCCCAGGCTGTTAGGGGTTATTGGTGTAAGGTACTTTCATATTTT
AAACAGAGGATATCGGCATTTGTTTCTTTCTCTGAGGACAAGAGA
AAAAAGCCAGGTTCCACAGAGGACACAGAGAAGGTTTGGGTGTCC
TCCTGGGGTTCTTTTTGCCAACTTTCCCCACGTTAAAGGTGAACA
TTGGTTCTTTCATTTGCTTTGGAAGTTTTAATCTCTAACAGTGGA
CAAAGTTACCAGTGCCTTAAACTCTGTTACACTTTTTGGAAGTGA
AAACTTTGTAGTATGATAGGTTATTTTGATGTAAAGATGTTCTGG
ATACCATTATATGTTCCCCCTGTTTCAGAGGCTCAGATTGTAATA
TGTAAATGGTATGTCATTCGCTACTATGATTTAATTTGAAATATG
GTCTTTTGGTTATGAATACTTTGCAGCACAGCTGAGAGGCTGTCT
GTTGTATTCATTGTGGTCATAGCACCTAACAACATTGTAGCCTCA
ATCGAGTGAGACAGACTAGAAGTTCCTAGTGATGGCTTATGATAG
CAAATGGCCTCATGTCAAATATTTAGATGTAATTTTGTGTAAGAA
ATACAGACTGGATGTACCACCAACTACTACCTGTAATGACAGGCC
TGTCCAACACATCTCCCTTTTCCATGACTGTGGTAGCCAGCATCG
GAAAGAACGCTGATTTAAAGAGGTCGCTTGGGAATTTTATTGACA
CAGTACCATTTAATGGGGAGGACAAAATGGGGCAGGGGAGGGAGA
AGTTTCTGTCGTTAAAAACAGATTTGGAAAGACTGGACTCTAAAG
TCTGTTGATTAAAGATGAGCTTTGTCTACTTCAAAAGTTTGTTTG
CTTACCCCTTCAGCCTCCAATTTTTTAAGTGAAAATATAGCTAAT
AACATGTGAAAAGAATAGAAGCTAAGGTTTAGATAAATATTGAGC
AGATCTATAGGAAGATTGAACCTGAATATTGCCATTATGCTTGAC
ATGGTTTCCAAAAAATGGTACTCCACATATTTCAGTGAGGGTAAG
TATTTTCCTGTTGTCAAGAATAGCATTGTAAAAGCATTTTGTAAT
AATAAAGAATAGCTTTAATGATATGCTTGTAACTAAAATAATTTT
GTAATGTATCAAATACATTTAAAACATTAAAATATAATCTCTATA
ATAAGAGCTCGCTGATCAGCCTCGACTGTGCCTTCTAGTTGCCAG
CCATCTGTTGTTTGCCCCTCCCCCGTGCCTTCCTTGACCCTGGAA
GGTGCCACTCCCACTGTCCTTTCCTAATAAAATGAGGAAATTGCA
TCGCATTGTCTGAGTAGGTGTCATTCTATTCTGGGGGGGGGGTGG
GGCAGGACAGCAAGGGGGAGGATTGGGAAGACAATAGCAGGCATG
CTGGGGATGCGGTGGGCTCTATGGAAGCTTGAATTCAGCTGACGT
GCCTCGGACCGCTAGGAACCCCTAGTGATGGAGTTGGCCACTCCC
TCTCTGCGCGCTCGCTCGCTCACTGAGGCCGGGCGACCAAAGGTC
GCCCGACGCCCGGGCTTTGCCCGGGCGGCCTCAGTGAGCGAGCGA
GCGCGCAGCTGCCTGCAGGGGCGCCTGATGCGGTATTTTCTCCTT
ACGCATCTGTGCGGTATTTCACACCGCATACGTCAAAGCAACCAT
AGTACGCGCCCTGTAGCGGCGCATTAAGCGCGGCGGGTGTGGTGG
TTACGCGCAGCGTGACCGCTACACTTGCCAGCGCCCTAGCGCCCG
CTCCTTTCGCTTTCTTCCCTTCCTTTCTCGCCACGTTCGCCGGCT
TTCCCCGTCAAGCTCTAAATCGGGGGCTCCCTTTAGGGTTCCGAT
TTAGTGCTTTACGGCACCTCGACCCCAAAAAACTTGATTTGGGTG
ATGGTTCACGTAGTGGGCCATCGCCCTGATAGACGGTTTTTCGCC
CTTTGACGTTGGAGTCCACGTTCTTTAATAGTGGACTCTTGTTCC
AAACTGGAACAACACTCAACCCTATCTCGGGCTATTCTTTTGATT
TATAAGGGATTTTGCCGATTTCGGCCTATTGGTTAAAAAATGAGC
TGATTTAACAAAAATTTAACGCGAATTTTAACAAAATATTAACGT
TTACAATTTTATGGTGCACTCTCAGTACAATCTGCTCTGATGCCG
CATAGTTAAGCCAGCCCCGACACCCGCCAACACCCGCTGACGCGC
CCTGACGGGCTTGTCTGCTCCCGGCATCCGCTTACAGACAAGCTG
TGACCGTCTCCGGGAGCTGCATGTGTCAGAGGTTTTCACCGTCAT
CACCGAAACGCGCGAGACGAAAGGGCCTCGTGATACGCCTATTTT
TATAGGTTAATGTCATGAACAATAAAACTGTCTGCTTACATAAAC
AGTAATACAAGGGGTGTTATGAGCCATATTCAACGGGAAACGTCG
AGGCCGCGATTAAATTCCAACATGGATGCTGATTTATATGGGTAT
AAATGGGCTCGCGATAATGTCGGGCAATCAGGTGCGACAATCTAT
CGCTTGTATGGGAAGCCCGATGCGCCAGAGTTGTTTCTGAAACAT
GGCAAAGGTAGCGTTGCCAATGATGTTACAGATGAGATGGTCAGA
CTAAACTGGCTGACGGAATTTATGCCTCTTCCGACCATCAAGCAT
TTTATCCGTACTCCTGATGATGCATGGTTACTCACCACTGCGATC
CCCGGAAAAACAGCATTCCAGGTATTAGAAGAATATCCTGATTCA
GGTGAAAATATTGTTGATGCGCTGGCAGTGTTCCTGCGCCGGTTG
CATTCGATTCCTGTTTGTAATTGTCCTTTTAACAGCGATCGCGTA
TTTCGTCTCGCTCAGGCGCAATCACGAATGAATAACGGTTTGGTT
GATGCGAGTGATTTTGATGACGAGCGTAATGGCTGGCCTGTTGAA
CAAGTCTGGAAAGAAATGCATAAACTTTTGCCATTCTCACCGGAT
TCAGTCGTCACTCATGGTGATTTCTCACTTGATAACCTTATTTTT
GACGAGGGGAAATTAATAGGTTGTATTGATGTTGGACGAGTCGGA
ATCGCAGACCGATACCAGGATCTTGCCATCCTATGGAACTGCCTC
GGTGAGTTTTCTCCTTCATTACAGAAACGGCTTTTTCAAAAATAT
GGTATTGATAATCCTGATATGAATAAATTGCAGTTTCATTTGATG
CTCGATGAGTTTTTCTAATCTCATGACCAAAATCCCTTAACGTGA
GTTTTCGTTCCACTGAGCGTCAGACCCCGTAGAAAAGATCAAAGG
ATCTTCTTGAGATCCTTTTTTTCTGCGCGTAATCTGCTGCTTGCA
AACAAAAAAACCACCGCTACCAGCGGTGGTTTGTTTGCCGGATCA
AGAGCTACCAACTCTTTTTCCGAAGGTAACTGGCTTCAGCAGAGC
GCAGATACCAAATACTGTCCTTCTAGTGTAGCCGTAGTTAGGCCA
CCACTTCAAGAACTCTGTAGCACCGCCTACATACCTCGCTCTGCT
AATCCTGTTACCAGTGGCTGCTGCCAGTGGCGATAAGTCGTGTCT
TACCGGGTTGGACTCAAGACGATAGTTACCGGATAAGGCGCAGCG
GTCGGGCTGAACGGGGGGTTCGTGCACACAGCCCAGCTTGGAGCG
AACGACCTACACCGAACTGAGATACCTACAGCGTGAGCTATGAGA
AAGCGCCACGCTTCCCGAAGGGAGAAAGGCGGACAGGTATCCGGT
AAGCGGCAGGGTCGGAACAGGAGAGCGCACGAGGGAGCTTCCAGG
GGGAAACGCCTGGTATCTTTATAGTCCTGTCGGGTTTCGCCACCT
CTGACTTGAGCGTCGATTTTTGTGATGCTCGTCAGGGGGGCGGAG
CCTATGGAAAAACGCCAGCAACGCGGCCTTTTTACGGTTCCTGGC
CTTTTGCTGGCCTTTTGCTCACATGT

TABLE 15
Components of Construct Sequence (SEQ ID NO: 106)

| Components | Position in construct |
|---|---|
| 5′ITR | 12-130 |
| Cloning site | 131-147 |
| TSPAN8 promoter | 148-1370 |
| hGJB2 minimal promoter | 1371-1498 |
| Cloning site | 1499-1507 |
| Synthetic barcode | 1508-1515 |
| 5′UTR | 1516-1877 |
| GJB2 (exon2) | 1889-2566 |
| 3xFLAG | 2579-2644 |
| 3′UTR (exon2) | 2648-4054 |
| bGHpA | 4076-4300 |
| Cloning site | 4301-4334 |
| 3′ITR | 4335-4464 |

Exemplary Construct sequence
(SEQ ID NO: 107)
CCTGCAGGCAGCTGCGCGCTCGCTCGCTCACTGAGGCCGCCCGGG
CGTCGGGCGACCTTTGGTCGCCCGGCCTCAGTGAGCGAGCGAGCG
CGCAGAGAGGGAGTGGCCAACTCCATCACTAGGGGTTCCTGCGGC
CGCACGCGTGGTATTCACAATGCATTCCCTCTGCCCACCACATTA
ATTATCAACTCCTTTTCCTGGCATTTACTCATCCAACGCATGGCC
CCACGTTAACTTTCAGTTCCCTTTCTCCCCTACAAATACTCCATA
ATCCAGCAACCCTGGGATCCCTGAGATGATGAAGAGGACCAGTGC
CCATTCCAGGAGACATCACCGCAGCCCTGAGGAATCGGCTATGGG
CACCAGCAGGGCACAGTGCCACACCTCGCCAATGCCTTGTCCTCC
TTTTCCATAGTGAGTCAGTCAGCAAGCGTGTAGAAGTGAGTTCCA
CACTCTCTTCCTCCCATAGGGAGATCACTTTTCTCATTCTAAGGG
TTCCAGGCACACTCACAATGGTGGCATTTGCTGAGCAGTGGCTTG
AATAAAGGGCTCTCAGAAAGCAAGATGTAACTCAGAGCATAGGCT
AGCCCCAGGAATGCTCTTGGGGAATGACCTGCAGCCTCCCAGTGA
AAGAGAGAATAAAAGAAAGCCCCAGCAGGCGAGCTGGGCAGTAGA
GAGTCCTGTAATTCCACCTTGGCAAGCACCATTTGCAAGAACGAA
CTGGGATAAGGTAAACAAAATATTGCCTAAAAGAGGCTTGTCCAA
AGAAGTCAGAATACGCTCTTCATTTACCTCTAAATTTCAGTACAC
CATAAATCTAAATACTCAAAAAAACCTGTGCCTTTTCAATCAAGG
TCAATTGCACGAATTCTTTTGGAAAACAGGACCTATGGCATTTCC
CAGACAAATCACCGTGAACCCTGTACTGTGCATTGCTGTCCTAAA
ATCCAAAGATTCTGTCATTTGTGTTACATAATTGCCTTTCATTTG
AACTCATTAATCAAATTGGGGTTTTTAAGCAACACCTAATTAATT
CTTTAACTGGCTCATCCACTGATCACTGAGTTCTATTTTGAAACT
ACGGACGTCGAGTTTCCTCTTTCACCCAGAATTTTCAGATCTTGT
TTAAAAAGTTGGGTGTGGTTTCATGGGGGGAGGGGGAAGAGCGAG
AGGAGACCAGAGGGACGGGGGCGGGGACTCTGCAAGAAAAACCTT
CCCGGTGCAATCGTGATCTAAGCTCTGAGGACCCAGAGGCCGGGC
GCGCTCCGCCCGCGGCGCCGCCCCCTCCGTAACTTTCCCAGTCTC
CGAGGGAAGAGGCGGGGTGTGGGGTGCGGTTAAAAGGCGCCACGG
CGGGAGACAGGTCTCACCGGTTTTCAGGTGTTGCGGCCCCGCAGC
GCCCGCGCGCTCCTCTCCCCGACTCGGAGCCCCTCGGCGGCGCCC
GGCCCAGGACCCGCCTAGGAGCGCAGGAGCCCCAGCGCAGAGACC
CCAACGCCGAGACCCCCGCCCCGGCCCCGCCGCGCTTCCTCCCGA
CGCAGTTTAGGACCCTTGTTCGCGAAGAGGTGGTGTGCGGCTGAG
ACCCGCGTCCTCAGGACGGTTCCATCAGTGCCTCGATCCTGCCCC
ACTGGAGGAGGAAGGCAGCCCGAACAGCGCTCACCTAACTAACAG
CTGCTGAGAGCTGGGTTCCGTGGCCATGCACCTGGGACTGCCTTG
AGAAGCGTGAGCAAACCGCCCAGAGTAGAAGCGCTAGCCACCATG
GATTGGGGCACGCTGCAGACGATCCTGGGGGGTGTGAACAAACAC
TCCACCAGCATTGGAAAGATCTGGCTCACCGTCCTCTTCATTTTT
CGCATTATGATCCTCGTTGTGGCTGCAAAGGAGGTGTGGGGAGAT
GAGCAGGCCGACTTTGTCTGCAACACCCTGCAGCCAGGCTGCAAG
AACGTGTGCTACGATCACTACTTCCCCATCTCCCACATCCGGCTA
TGGGCCCTGCAGCTGATCTTCGTGTCCACGCCAGCGCTCCTAGTG
GCCATGCACGTGGCCTACCGGAGACATGAGAAGAAGAGGAAGTTC
ATCAAGGGGGAGATAAAGAGTGAATTTAAGGACATCGAGGAGATC
AAAACCCAGAAGGTCCGCATCGAAGGCTCCCTGTGGTGGACCTAC
ACAAGCAGCATCTTCTTCCGGGTCATCTTCGAAGCCGCCTTCATG
TACGTCTTCTATGTCATGTACGACGGCTTCTCCATGCAGCGGCTG
GTGAAGTGCAACGCCTGGCCTTGTCCCAACACTGTGGACTGCTTT
GTGTCCCGGCCCACGGAGAAGACTGTCTTCACAGTGTTCATGATT
GCAGTGTCTGGAATTTGCATCCTGCTGAATGTCACTGAATTGTGT
TATTTGCTAATTAGATATTGTTCTGGGAAGTCAAAAAAGCCAGTT
GGATCCCGGGCTGACTACAAAGACCATGACGGTGATTATAAAGAT
CATGACATCGACTACAAGGATGACGATGACAAGTAAGAAATAGAC
AGCATGAGAGGGATGAGGCAACCCGTGCTCAGCTGTCAAGGCTCA
GTCGCTAGCATTTCCCAACACAAAGATTCTGACCTTAAATGCAAC
CATTTGAAACCCCTGTAGGCCTCAGGTGAAACTCCAGATGCCACA
ATGGAGCTCTGCTCCCCTAAAGCCTCAAAACAAAGGCCTAATTCT
ATGCCTGTCTTAATTTTCTTTCACTTAAGTTAGTTCCACTGAGAC
CCCAGGCTGTTAGGGGTTATTGGTGTAAGGTACTTTCATATTTTA
AACAGAGGATATCGGCATTTGTTTCTTTCTCTGAGGACAAGAGAA
AAAAGCCAGGTTCCACAGAGGACACAGAGAAGGTTTGGGTGTCCT
CCTGGGGTTCTTTTTGCCAACTTTCCCCACGTTAAAGGTGAACAT
TGGTTCTTTCATTTGCTTTGGAAGTTTTAATCTCTAACAGTGGAC
AAAGTTACCAGTGCCTTAAACTCTGTTACACTTTTTGGAAGTGAA
AACTTTGTAGTATGATAGGTTATTTTGATGTAAAGATGTTCTGGA
TACCATTATATGTTCCCCCTGTTTCAGAGGCTCAGATTGTAATAT
GTAAATGGTATGTCATTCGCTACTATGATTTAATTTGAAATATGG
TCTTTTGGTTATGAATACTTTGCAGCACAGCTGAGAGGCTGTCTG
TTGTATTCATTGTGGTCATAGCACCTAACAACATTGTAGCCTCAA
TCGAGTGAGACAGACTAGAAGTTCCTAGTGATGGCTTATGATAGC
AAATGGCCTCATGTCAAATATTTAGATGTAATTTTGTGTAAGAAA
TACAGACTGGATGTACCACCAACTACTACCTGTAATGACAGGCCT
GTCCAACACATCTCCCTTTTCCATGACTGTGGTAGCCAGCATCGG
AAAGAACGCTGATTTAAAGAGGTCGCTTGGGAATTTTATTGACAC
AGTACCATTTAATGGGGAGGACAAAATGGGGCAGGGGAGGGAGAA
GTTTCTGTCGTTAAAAACAGATTTGGAAAGACTGGACTCTAAAGT
CTGTTGATTAAAGATGAGCTTTGTCTACTTCAAAAGTTTGTTTGC
TTACCCCTTCAGCCTCCAATTTTTTAAGTGAAAATATAGCTAATA
ACATGTGAAAAGAATAGAAGCTAAGGTTTAGATAAATATTGAGCA
GATCTATAGGAAGATTGAACCTGAATATTGCCATTATGCTTGACA
TGGTTTCCAAAAAATGGTACTCCACATATTTCAGTGAGGGTAAGT
ATTTTCCTGTTGTCAAGAATAGCATTGTAAAAGCATTTTGTAATA
ATAAAGAATAGCTTTAATGATATGCTTGTAACTAAAATAATTTTG
TAATGTATCAAATACATTTAAAACATTAAAATATAATCTCTATAA
TAAGAGCTCGCTGATCAGCCTCGACTGTGCCTTCTAGTTGCCAGC
CATCTGTTGTTTGCCCCTCCCCCGTGCCTTCCTTGACCCTGGAAG
GTGCCACTCCCACTGTCCTTTCCTAATAAAATGAGGAAATTGCAT
CGCATTGTCTGAGTAGGTGTCATTCTATTCTGGGGGGTGGGGTGG
GGCAGGACAGCAAGGGGGAGGATTGGGAAGACAATAGCAGGCATG
CTGGGGATGCGGTGGGCTCTATGGAAGCTTGAATTCAGCTGACGT
GCCTCGGACCGCTAGGAACCCCTAGTGATGGAGTTGGCCACTCCC
TCTCTGCGCGCTCGCTCGCTCACTGAGGCCGGGCGACCAAAGGTC
GCCCGACGCCCGGGCTTTGCCCGGGCGGCCTCAGTGAGCGAGCGA
GCGCGCAGCTGCCTGCAGGGGCGCCTGATGCGGTATTTTCTCCTT
ACGCATCTGTGCGGTATTTCACACCGCATACGTCAAAGCAACCAT
AGTACGCGCCCTGTAGCGGCGCATTAAGCGCGGCGGGTGTGGTGG
TTACGCGCAGCGTGACCGCTACACTTGCCAGCGCCCTAGCGCCCG
CTCCTTTCGCTTTCTTCCCTTCCTTTCTCGCCACGTTCGCCGGCT
TTCCCCGTCAAGCTCTAAATCGGGGGCTCCCTTTAGGGTTCCGAT
TTAGTGCTTTACGGCACCTCGACCCCAAAAAACTTGATTTGGGTG
ATGGTTCACGTAGTGGGCCATCGCCCTGATAGACGGTTTTTCGCC
CTTTGACGTTGGAGTCCACGTTCTTTAATAGTGGACTCTTGTTCC
AAACTGGAACAACACTCAACCCTATCTCGGGCTATTCTTTTGATT
TATAAGGGATTTTGCCGATTTCGGCCTATTGGTTAAAAAATGAGC
TGATTTAACAAAAATTTAACGCGAATTTTAACAAAATATTAACGT
TTACAATTTTATGGTGCACTCTCAGTACAATCTGCTCTGATGCCG
CATAGTTAAGCCAGCCCCGACACCCGCCAACACCCGCTGACGCGC
CCTGACGGGCTTGTCTGCTCCCGGCATCCGCTTACAGACAAGCTG
TGACCGTCTCCGGGAGCTGCATGTGTCAGAGGTTTTCACCGTCAT
CACCGAAACGCGCGAGACGAAAGGGCCTCGTGATACGCCTATTTT
TATAGGTTAATGTCATGAACAATAAAACTGTCTGCTTACATAAAC
AGTAATACAAGGGGTGTTATGAGCCATATTCAACGGGAAACGTCG
AGGCCGCGATTAAATTCCAACATGGATGCTGATTTATATGGGTAT
AAATGGGCTCGCGATAATGTCGGGCAATCAGGTGCGACAATCTAT
CGCTTGTATGGGAAGCCCGATGCGCCAGAGTTGTTTCTGAAACAT
GGCAAAGGTAGCGTTGCCAATGATGTTACAGATGAGATGGTCAGA
CTAAACTGGCTGACGGAATTTATGCCTCTTCCGACCATCAAGCAT
TTTATCCGTACTCCTGATGATGCATGGTTACTCACCACTGCGATC
CCCGGAAAAACAGCATTCCAGGTATTAGAAGAATATCCTGATTCA
GGTGAAAATATTGTTGATGCGCTGGCAGTGTTCCTGCGCCGGTTG
CATTCGATTCCTGTTTGTAATTGTCCTTTTAACAGCGATCGCGTA
TTTCGTCTCGCTCAGGCGCAATCACGAATGAATAACGGTTTGGTT
GATGCGAGTGATTTTGATGACGAGCGTAATGGCTGGCCTGTTGAA
CAAGTCTGGAAAGAAATGCATAAACTTTTGCCATTCTCACCGGAT
TCAGTCGTCACTCATGGTGATTTCTCACTTGATAACCTTATTTTT
GACGAGGGGAAATTAATAGGTTGTATTGATGTTGGACGAGTCGGA
ATCGCAGACCGATACCAGGATCTTGCCATCCTATGGAACTGCCTC
GGTGAGTTTTCTCCTTCATTACAGAAACGGCTTTTTCAAAAATAT
GGTATTGATAATCCTGATATGAATAAATTGCAGTTTCATTTGATG
CTCGATGAGTTTTTCTAATCTCATGACCAAAATCCCTTAACGTGA
GTTTTCGTTCCACTGAGCGTCAGACCCCGTAGAAAAGATCAAAGG
ATCTTCTTGAGATCCTTTTTTTCTGCGCGTAATCTGCTGCTTGCA
AACAAAAAAACCACCGCTACCAGCGGTGGTTTGTTTGCCGGATCA
AGAGCTACCAACTCTTTTTCCGAAGGTAACTGGCTTCAGCAGAGC
GCAGATACCAAATACTGTCCTTCTAGTGTAGCCGTAGTTAGGCCA
CCACTTCAAGAACTCTGTAGCACCGCCTACATACCTCGCTCTGCT
AATCCTGTTACCAGTGGCTGCTGCCAGTGGCGATAAGTCGTGTCT
TACCGGGTTGGACTCAAGACGATAGTTACCGGATAAGGCGCAGCG
GTCGGGCTGAACGGGGGGTTCGTGCACACAGCCCAGCTTGGAGCG
AACGACCTACACCGAACTGAGATACCTACAGCGTGAGCTATGAGA
AAGCGCCACGCTTCCCGAAGGGAGAAAGGCGGACAGGTATCCGGT
AAGCGGCAGGGTCGGAACAGGAGAGCGCACGAGGGAGCTTCCAGG
GGGAAACGCCTGGTATCTTTATAGTCCTGTCGGGTTTCGCCACCT
CTGACTTGAGCGTCGATTTTTGTGATGCTCGTCAGGGGGGCGGAG
CCTATGGAAAAACGCCAGCAACGCGGCCTTTTTACGGTTCCTGGC
CTTTTGCTGGCCTTTTGCTCACATGT

TABLE 16
Components of Construct Sequence (SEQ ID NO: 107)

| Components | Position in construct |
|---|---|
| 5′ITR | 12-130 |
| Cloning site | 131-147 |
| VIM promoter | 148-1234 |
| hGJB2 minimal promoter | 1235-1362 |
| Cloning site | 1363-1371 |
| Synthetic barcode | 1372-1379 |
| 5′UTR | 1380-1741 |
| GJB2 (exon2) | 1753-2430 |
| 3xFLAG | 2443-2508 |
| 3′UTR (exon2) | 2512-3918 |
| bGHpA | 3940-4164 |
| Cloning site | 4165-4198 |
| 3′ITR | 4199-4328 |

### Pharmaceutical Compositions

Among other things, the present disclosure provides pharmaceutical compositions. In some aspects, compositions provided herein are suitable for administration to an animal for the amelioration of symptoms associated with syndromic and/or nonsyndromic hearing loss.

In some aspects, pharmaceutical compositions of the present disclosure may comprise, e.g., a polynucleotide, e.g., one or more constructs, as described herein. In some aspects, a pharmaceutical composition may comprise one or more AAV particles, e.g., one or more rAAV construct encapsidated by one or more AAV serotype capsids, as described herein.

In some aspects, a pharmaceutical composition comprises one or more pharmaceutically or physiologically acceptable carriers, diluents or excipients. As used herein, the term “pharmaceutically acceptable carrier” includes solvents, dispersion media, coatings, antibacterial agents, antifungal agents, and the like that are compatible with pharmaceutical administration. Supplementary active compounds can also be incorporated into any of the compositions described herein. Such compositions may include one or more buffers, such as neutral-buffered saline, phosphate-buffered saline, and the like; one or more carbohydrates, such as glucose, mannose, sucrose, and dextran; mannitol; one or more proteins, polypeptides, or amino acids, such as glycine; one or more antioxidants; one or more chelating agents, such as EDTA or glutathione; and/or one or more preservatives. In some aspects, formulations are in a dosage forms, such as injectable solutions, injectable gels, drug-release capsules, and the like.

In some aspects, compositions of the present disclosure are formulated for intravenous administration. In some aspects compositions of the present disclosure are formulated for intra-cochlear administration. In some aspects, a composition (e.g., a therapeutic composition) is formulated to comprise a lipid nanoparticle, a polymeric nanoparticle, a mini-circle DNA and/or a CELiD DNA.

In some aspects, a composition disclosed herein is formulated as a sterile suspension for intracochlear administration. In some aspects, a composition comprises constructs in an amount of at least 1E11, at least 5E11, at least 1E12, at least 5E12, at least 1E13, at least 2E13, at least 3E13, at least 4E13, at least 5E13, at least 6E13, at least 7E13, at least 8E13, at least 9E13, or at least 1E14 vector genomes (vg) per milliliter (mL). In some aspects, a composition comprises constructs in an amount of at most 1E15, at most 5E14, at most 1E14, at most 5E13, at most 1E13, at most 9E12, at most 8E12, at most 7E12, at most 6E12, at most 5E12, at most 4E12, at most 3E12, at most 2E12, or at most 1E12 vector genomes (vg) per milliliter (mL). In some aspects, a composition comprises constructs in an amount of 1E12 to 1E13, 5E12 to 5E13, or 1E13 to 2E13 vector genomes (vg) per milliliter (mL).

In some aspects, a therapeutic composition is formulated to comprise a synthetic perilymph solution. For example, in some aspects, a synthetic perilymph solution includes 20-200 mM NaCl; 1-5 mM KCl; 0.1-10 mM CaCl2; 1-10 mM glucose; and 2-50 mM HEPES, with a pH between about 6 and about 9. In some aspects, a therapeutic composition is formulated to comprise a physiologically suitable solution. For example, in some aspects, a physiologically suitable solution comprises commercially available 1xPBS with pluronic acid F68, prepared to a final concentration of: 8.10 mM Sodium Phosphate Dibasic, 1.5 mM Monopotassium Phosphate, 2.7 mM Potassium Chloride, 172 mM Sodium Chloride, and 0.001% Pluronic Acid F68). In some aspects, alternative pluronic acids are utilized. In some aspects, alternative ion concentrations are utilized.

In some aspects, any of the pharmaceutical compositions described herein may further comprise one or more agents that promote the entry of a nucleic acid or any of the constructs described herein into a mammalian cell (e.g., a liposome or cationic lipid). In some aspects, any of the constructs described herein can be formulated using natural and/or synthetic polymers. Non-limiting examples of polymers that may be included in any of the compositions described herein can include, but are not limited to, DYNAMIC POLYCONJUGATE® (Arrowhead Research Corp., Pasadena, Calif.), formulations from Minis Bio (Madison, Wis.) and Roche Madison (Madison, Wis.), PhaseRX polymer formulations such as, without limitation, SMARTT POLYMER TECHNOLOGY® (PhaseRX, Seattle, Wash.), DM:RI/DOPE, poloxamer, VAXFECTIN® adjuvant from Vical (San Diego, Calif.), chitosan, cyclodextrin from Calando Pharmaceuticals (Pasadena, Calif.), dendrimers and poly (lactic-co-glycolic acid) (PLGA) polymers, RONDEL™ (RNAi/Oligonucleotide Nanoparticle Delivery) polymers (Arrowhead Research Corporation, Pasadena, Calif), and pH responsive co-block polymers, such as, but not limited to, those produced by PhaseRX (Seattle, Wash.). Many of these polymers have demonstrated efficacy in delivering oligonucleotides in vivo into a mammalian cell (see, e.g., deFougerolles, Human Gene Ther. 19:125-132, 2008; Rozema et al., Proc. Natl. Acad. Sci. U.S.A. 104:12982-12887, 2007; Rozema et al., Proc. Natl. Acad. Sci. U.S.A. 104:12982-12887, 2007; Hu-Lieskovan et al., Cancer Res. 65:8984-8982, 2005; Heidel et al., Proc. Natl. Acad. Sci. U.S.A. 104:5715-5721, 2007, each of which is incorporated in its entirety herein by reference).

In some aspects, a composition includes a pharmaceutically acceptable carrier (e.g., phosphate buffered saline, saline, or bacteriostatic water). Upon formulation, solutions will be administered in a manner compatible with a dosage formulation and in such amount as is therapeutically effective. Formulations are easily administered in a variety of dosage forms such as injectable solutions, injectable gels, drug-release capsules, and the like.

In some aspects, a composition provided herein can be, e.g., formulated to be compatible with their intended route of administration. A non-limiting example of an intended route of administration is local administration (e.g., intra-cochlear administration). In some aspects, a provided composition comprises one nucleic acid construct. In some aspects, a provided composition comprises two or more different constructs. In some aspects, a composition that include a single nucleic acid construct comprising a coding sequence that encodes a polypeptide (e.g., a therapeutic polypeptide) and/or a functional characteristic portion thereof. In some aspects, compositions comprise a single nucleic acid construct comprising a coding sequence that encodes a polypeptide (e.g., a therapeutic polypeptide) and/or a functional characteristic portion thereof, which, when introduced into a mammalian cell, that coding sequence is integrated into the genome of the mammalian cell.

Also provided are kits including any of the compositions described herein. In some aspects, a kit can include a solid composition (e.g., a lyophilized composition including the at least two different constructs described herein) and a liquid for solubilizing the lyophilized composition. In some aspects, a kit can include a pre-loaded syringe including any of the compositions described herein.

In some aspects, the kit includes a vial comprising any of the compositions described herein (e.g., formulated as an aqueous composition, e.g., an aqueous pharmaceutical composition).

In some aspects, the kits can include instructions for performing any of the methods described herein.

### Genetically Modified Cells

The present disclosure also provides a cell (e.g., an animal cell, e.g., a mammalian cell, e.g., a primate cell, e.g., a human cell) that includes any of the nucleic acids, constructs or compositions described herein. In some aspects, an animal cell is a human cell (e.g., a human supporting cell or a human hair cell). In other aspects, an animal cell is a non-human mammal (e.g., Simian cell, Felidae cell, Canidae cell etc.). A person skilled in the art will appreciate that the nucleic acids and constructs described herein can be introduced into any animal cell (e.g., the supporting or hair cells of any animal suitable for veterinary intervention). Non-limiting examples of constructs and methods for introducing constructs into animal cells are described herein.

In some aspects, an animal cell can be any cell of the inner ear, including hair and/or supporting cells. Non-limiting examples such cells include: Hensen's cells, Deiters' cells, cells of the endolymphatic sac and duct, transitional cells in the saccule, utricle, and ampulla, inner and outer hair cells, spiral ligament cells, spiral ganglion cells, spiral prominence cells, external saccule cells, marginal cells, intermediate cells, basal cells, inner pillar cells, outer pillar cells, Claudius cells, inner border cells, inner phalangeal cells, or cells of the stria vascularis.

In some aspects, an animal cell is a specialized cell of the cochlea. In some aspects, an animal cell is a hair cell. In some aspects, an animal cell is a cochlear inner hair cell or a cochlear outer hair cell. In some aspects, an animal cell is a cochlear inner hair cell. In some aspects, an animal cell is a cochlear outer hair cell.

In some aspects, an animal cell is in vitro. In some aspects, an animal cell is of a cell type which is endogenously present in an animal, e.g., in a primate and/or human. In some aspects, an animal cell is an autologous cell obtained from an animal and cultured ex vivo. In some aspects, the ex vivo cell is an inner ear cell. In some aspects, the ex vivo cell is an inner ear supporting cell. In some aspects, the ex vivo cell is an inner phalangeal cells/border cells (IPhC), inner pillar cells (IPC), outer pillar cells (OPC), Deiters' cells rows 1 and 2 (DC1/2), Deiters' cells row 3 (DC3), Hensen's cells (Hec), Claudius cells/outer sulcus cells (CC/OSC), interdental cells (Idc), inner sulcus cells (ISC), Kölliker's organ cells (KO), Lateral greater epithelial ridge cells (LGER), and OC90+ cells (OC90). In some aspects, the ex vivo cell is an inner phalangeal cells/border cells (IPhC), inner pillar cells (IPC), outer pillar cells (OPC), Deiters' cells rows 1 and 2 (DC1/2), Deiters' cells row 3 (DC3), Hensen's cells (Hec), Claudius cells/outer sulcus cells (CC/OSC), interdental cells (Idc), inner sulcus cells (ISC), Kölliker's organ cells (KO), greater ridge epithelial ridge cells (GER) (including lateral greater epithelial ridge cells (LGER)), and OC90+ cells (OC90), fibroblasts, and other cells of the lateral wall.

### Methods

Among other things, the present disclosure provides methods. In some aspects, a method comprises introducing a composition, construct, or polynucleotide as described herein into the inner ear (e.g., a cochlea) of a subject. For example, provided herein are methods that in some aspects include administering to an inner ear (e.g., cochlea) of a subject (e.g., an animal, e.g., a mammal, e.g., a primate, e.g., a human) a therapeutically effective amount of any composition, construct, or polynucleotide described herein. In some aspects of any of these methods, the subject has been previously identified as having a defective inner ear cell target gene (e.g., a supporting and/or hearing cell target gene having a mutation that results in a decrease in the expression and/or activity of a supporting and/or hearing cell target protein encoded by the gene). Some aspects of any of these methods further include, prior to the introducing or administering step, determining that the subject has a defective inner ear cell target gene. Some aspects of any of these methods can further include detecting a mutation in an inner ear cell target gene in a subject. Some aspects of any of the methods can further include identifying or diagnosing a subject as having nonsyndromic or syndromic sensorineural hearing loss.

In some aspects, provided herein are methods of correcting an inner ear cell target gene defect in an inner ear of a subject, e.g., an animal, e.g., a mammal, e.g., a primate, e.g., a human. In some aspects, methods include administering to the inner ear of a subject a therapeutically effective amount of any of the compositions described herein, where the administering repairs and or ameliorates the inner ear cell target gene defect in any cell subset of the inner ear of a subject. In some aspects, the inner ear target cell may be a sensory cell, e.g., a hair cell, and/or a non-sensory cell, e.g., a supporting cell, and/or all or any subset of inner ear cells.

Also provided herein are methods of promoting expression (e.g., increasing the expression level) of an inner ear cell target protein in any subset of inner ear cells of a subject (e.g., an animal, e.g., a mammal, e.g., a primate, e.g., a human) that include: administering to the inner ear of the subject a therapeutically effective amount of any of the compositions described herein, where the administering results in an increase in the expression level of the inner ear cell target protein (e.g., a polypeptide (e.g., a therapeutic polypeptide)) in any cell subset of the inner ear of a subject. In some aspects, the inner ear target cell may be a sensory cell, e.g., a hair cell, and/or a non-sensory cell, e.g., a supporting cell, and/or all or any subset of inner ear cells.

Also provided herein are methods of treating hearing loss, e.g., nonsyndromic sensorineural hearing loss or syndromic sensorineural hearing loss, in a subject (e.g., an animal, e.g., a mammal, e.g., a primate, e.g., a human) identified as having a defective inner ear cell target gene that include: administering to the inner ear of the subject a therapeutically effective amount of any of the compositions described herein.

Also provided herein are methods of restoring synapses and/or preserving spiral ganglion nerves in a subject identified or diagnosed as having an inner ear disorder that include: administering to the inner ear of the subject a therapeutically effective amount of any of the compositions described herein.

Also provided herein are methods of reducing the size of, and/or restoring the vestibular aqueduct to an appropriate size. Also provided herein are methods of restoring endolymphatic pH to an appropriate and/or acceptable level in a subject identified or diagnosed as having an inner ear disorder that include: administering to the inner ear of the subject a therapeutically effective amount of any of the compositions described herein.

Also provided herein are methods of expressing a polypeptide (e.g., a therapeutic polypeptide) in an inner ear supporting cell of a subject in need thereof. In some aspects, the inner ear supporting cells are selected from one or more of inner phalangeal cells/border cells (IPhC), inner pillar cells (IPC), outer pillar cells (OPC), Deiters' cells rows 1 and 2 (DC1/2), Deiters' cells row 3 (DC3), Hensen's cells (Hec), Claudius cells/outer sulcus cells (CC/OSC), interdental cells (Idc), inner sulcus cells (ISC), Kölliker's organ cells (KO), Lateral greater epithelial ridge cells (LGER), and OC90+ cells (OC90). In some aspects, the inner ear supporting cells are selected from one or more of inner phalangeal cells/border cells (IPhC), inner pillar cells (IPC), outer pillar cells (OPC), Deiters' cells rows 1 and 2 (DC1/2), Deiters' cells row 3 (DC3), Hensen's cells (Hec), Claudius cells/outer sulcus cells (CC/OSC), interdental cells (Idc), inner sulcus cells (ISC), Kölliker's organ cells (KO), greater ridge epithelial ridge cells (GER) (including lateral greater epithelial ridge cells (LGER)), and OC90+ cells (OC90), fibroblasts, and other cells of the lateral wall.

In some aspects, expression of the polypeptide (e.g., therapeutic polypeptide) is reduced, suppressed, or eliminated in inner ear hair cells, spiral ganglion cells, lateral supporting cells, basilar membrane cells, medial supporting cells, spiral limbus cells, or any combination thereof. In some aspects, toxicity due to expression of the therapeutic polypeptide is reduced in inner ear hair cells, spiral ganglion cells, lateral supporting cells, basilar membrane cells, medial supporting cells, spiral limbus cells, or any combination thereof. In some aspects, the therapeutic protein is predominately expressed in inner ear supporting cells. In some aspects, the inner ear supporting cells are selected from one or more of inner phalangeal cells/border cells (IPhC), inner pillar cells (IPC), outer pillar cells (OPC), Deiters' cells rows 1 and 2 (DC1/2), Deiters' cells row 3 (DC3), Hensen's cells (Hec), Claudius cells/outer sulcus cells (CC/OSC), interdental cells (Idc), inner sulcus cells (ISC), Kölliker's organ cells (KO), greater ridge epithelial ridge cells (GER) (including lateral greater epithelial ridge cells (LGER)), and OC90+ cells (OC90), fibroblasts, and other cells of the lateral wall.

In some aspects, expression of the polypeptide is reduced, suppressed, or eliminated in inner ear hair cells, spiral ganglion cells, lateral supporting cells, basilar membrane cells, medial supporting cells, spiral limbus cells, or any combination thereof. In some aspects, toxicity due to expression of the polypeptide is reduced in inner ear hair cells, spiral ganglion cells, lateral supporting cells, basilar membrane cells, medial supporting cells, spiral limbus cells, or any combination thereof. In some aspects, the protein is predominately expressed in inner ear supporting cells. In some aspects, the inner ear supporting cells are selected from one or more of inner phalangeal cells/border cells (IPhC), inner pillar cells (IPC), outer pillar cells (OPC), Deiters' cells rows 1 and 2 (DC1/2), Deiters' cells row 3 (DC3), Hensen's cells (Hec), Claudius cells/outer sulcus cells (CC/OSC), interdental cells (Idc), inner sulcus cells (ISC), Kölliker's organ cells (KO), greater ridge epithelial ridge cells (GER) (including lateral greater epithelial ridge cells (LGER)), and OC90+ cells (OC90), fibroblasts, and other cells of the lateral wall.

In some aspects, administration is to the inner ear of the subject. In some aspects, the administration is to the cochlea of the subject. In some aspects, the administration is via a round window membrane injection.

Also provided herein are methods of increasing expression of a therapeutic polypeptide in an inner ear supporting cell of a subject in need thereof. In some aspects, the expression of the therapeutic polypeptide is reduced, suppressed, or eliminated in non-inner ear supporting cells. In some aspects, the inner ear supporting cells are selected from one or more of inner phalangeal cells/border cells (IPhC), inner pillar cells (IPC), outer pillar cells (OPC), Deiters' cells rows 1 and 2 (DC1/2), Deiters' cells row 3 (DC3), Hensen's cells (Hec), Claudius cells/outer sulcus cells (CC/OSC), interdental cells (Idc), inner sulcus cells (ISC), Kölliker's organ cells (KO), greater ridge epithelial ridge cells (GER) (including lateral greater epithelial ridge cells (LGER)), and OC90+ cells (OC90), fibroblasts, and other cells of the lateral wall. In some aspects, expression of the therapeutic polypeptide is reduced, suppressed, or eliminated in inner ear hair cells, spiral ganglion cells, lateral supporting cells, basilar membrane cells, medial supporting cells, spiral limbus cells, or any combination thereof. In some aspects, toxicity due to expression of the therapeutic polypeptide is reduced in inner ear hair cells, spiral ganglion cells, lateral supporting cells, basilar membrane cells, medial supporting cells, spiral limbus cells, or any combination thereof. In some aspects, the therapeutic protein is predominately expressed in inner ear supporting cells. In some aspects, the inner ear supporting cells are selected from one or more of inner phalangeal cells/border cells (IPhC), inner pillar cells (IPC), outer pillar cells (OPC), Deiters' cells rows 1 and 2 (DC1/2), Deiters' cells row 3 (DC3), Hensen's cells (Hec), Claudius cells/outer sulcus cells (CC/OSC), interdental cells (Idc), inner sulcus cells (ISC), Kölliker's organ cells (KO), greater ridge epithelial ridge cells (GER) (including lateral greater epithelial ridge cells (LGER)), and OC90+ cells (OC90), fibroblasts, and other cells of the lateral wall. Also provided herein are methods of promoting expression (e.g., increasing expression) of a polypeptide in an inner ear supporting cell of a subject in need thereof. In some aspects, the expression of the polypeptide is reduced, suppressed, or eliminated in non-inner ear supporting cells. In some aspects, the inner ear supporting cells are selected from one or more of inner phalangeal cells/border cells (IPhC), inner pillar cells (IPC), outer pillar cells (OPC), Deiters' cells rows 1 and 2 (DC1/2), Deiters' cells row 3 (DC3), Hensen's cells (Hec), Claudius cells/outer sulcus cells (CC/OSC), interdental cells (Idc), inner sulcus cells (ISC), Kölliker's organ cells (KO), greater ridge epithelial ridge cells (GER) (including lateral greater epithelial ridge cells (LGER)), and OC90+ cells (OC90), fibroblasts, and other cells of the lateral wall. In some aspects, expression of the polypeptide is reduced, suppressed, or eliminated in inner ear hair cells, spiral ganglion cells, lateral supporting cells, basilar membrane cells, medial supporting cells, spiral limbus cells, or any combination thereof. In some aspects, toxicity due to expression of the polypeptide is reduced in inner ear hair cells, spiral ganglion cells, lateral supporting cells, basilar membrane cells, medial supporting cells, spiral limbus cells, or any combination thereof. In some aspects, the protein is predominately expressed in inner ear supporting cells. In some aspects, the inner ear supporting cells are selected from one or more of inner phalangeal cells/border cells (IPhC), inner pillar cells (IPC), outer pillar cells (OPC), Deiters' cells rows 1 and 2 (DC1/2), Deiters' cells row 3 (DC3), Hensen's cells (Hec), Claudius cells/outer sulcus cells (CC/OSC), interdental cells (Idc), inner sulcus cells (ISC), Kölliker's organ cells (KO), greater ridge epithelial ridge cells (GER) (including lateral greater epithelial ridge cells (LGER)), and OC90+ cells (OC90), fibroblasts, and other cells of the lateral wall.

In some aspects, administration is to the inner ear of the subject. In some aspects, the administration is to the cochlea of the subject. In some aspects, the administration is via a round window membrane injection.

Also provided herein are methods of decreasing expression of the therapeutic polypeptide in non-inner ear supporting cells. In some aspects, the inner ear supporting cells are selected from one or more of inner phalangeal cells/border cells (IPhC), inner pillar cells (IPC), outer pillar cells (OPC), Deiters' cells rows 1 and 2 (DC1/2), Deiters' cells row 3 (DC3), Hensen's cells (Hec), Claudius cells/outer sulcus cells (CC/OSC), interdental cells (Idc), inner sulcus cells (ISC), Kölliker's organ cells (KO), Lateral greater epithelial ridge cells (LGER), and OC90+ cells (OC90). In some aspects, the inner ear supporting cells are selected from one or more of inner phalangeal cells/border cells (IPhC), inner pillar cells (IPC), outer pillar cells (OPC), Deiters' cells rows 1 and 2 (DC1/2), Deiters' cells row 3 (DC3), Hensen's cells (Hec), Claudius cells/outer sulcus cells (CC/OSC), interdental cells (Idc), inner sulcus cells (ISC), Kölliker's organ cells (KO), greater ridge epithelial ridge cells (GER) (including lateral greater epithelial ridge cells (LGER)), and OC90+ cells (OC90), fibroblasts, and other cells of the lateral wall.

Also provided herein are methods of decreasing expression of the polypeptide in non-inner ear supporting cells. In some aspects, the inner ear supporting cells are selected from one or more of inner phalangeal cells/border cells (IPhC), inner pillar cells (IPC), outer pillar cells (OPC), Deiters' cells rows 1 and 2 (DC1/2), Deiters' cells row 3 (DC3), Hensen's cells (Hec), Claudius cells/outer sulcus cells (CC/OSC), interdental cells (Idc), inner sulcus cells (ISC), Kölliker's organ cells (KO), Lateral greater epithelial ridge cells (LGER), and OC90+ cells (OC90). In some aspects, the inner ear supporting cells are selected from one or more of inner phalangeal cells/border cells (IPhC), inner pillar cells (IPC), outer pillar cells (OPC), Deiters' cells rows 1 and 2 (DC1/2), Deiters' cells row 3 (DC3), Hensen's cells (Hec), Claudius cells/outer sulcus cells (CC/OSC), interdental cells (Idc), inner sulcus cells (ISC), Kölliker's organ cells (KO), greater ridge epithelial ridge cells (GER) (including lateral greater epithelial ridge cells (LGER)), and OC90+ cells (OC90), fibroblasts, and other cells of the lateral wall.

In some aspects, expression of the therapeutic polypeptide is reduced, suppressed, or eliminated in inner ear hair cells, spiral ganglion cells, lateral supporting cells, basilar membrane cells, medial supporting cells, spiral limbus cells, or any combination thereof. In some aspects, toxicity due to expression of the therapeutic polypeptide is reduced in inner ear hair cells, spiral ganglion cells, lateral supporting cells, basilar membrane cells, medial supporting cells, spiral limbus cells, or any combination thereof. In some aspects, the therapeutic protein is predominately expressed in inner ear supporting cells. In some aspects, the inner ear supporting cells are selected from one or more of inner phalangeal cells/border cells (IPhC), inner pillar cells (IPC), outer pillar cells (OPC), Deiters' cells rows 1 and 2 (DC1/2), Deiters' cells row 3 (DC3), Hensen's cells (Hec), Claudius cells/outer sulcus cells (CC/OSC), interdental cells (Idc), inner sulcus cells (ISC), Kölliker's organ cells (KO), greater ridge epithelial ridge cells (GER) (including lateral greater epithelial ridge cells (LGER)), and OC90+ cells (OC90), fibroblasts, and other cells of the lateral wall. In some aspects, administration is to the inner ear of the subject. In some aspects, the administration is to the cochlea of the subject. In some aspects, the administration is via a round window membrane injection.

In some aspects, expression of the polypeptide is reduced, suppressed, or eliminated in inner ear hair cells, spiral ganglion cells, lateral supporting cells, basilar membrane cells, medial supporting cells, spiral limbus cells, or any combination thereof. In some aspects, toxicity due to expression of the polypeptide is reduced in inner ear hair cells, spiral ganglion cells, lateral supporting cells, basilar membrane cells, medial supporting cells, spiral limbus cells, or any combination thereof. In some aspects, the protein is predominately expressed in inner ear supporting cells. In some aspects, the inner ear supporting cells are selected from one or more of inner phalangeal cells/border cells (IPhC), inner pillar cells (IPC), outer pillar cells (OPC), Deiters' cells rows 1 and 2 (DC1/2), Deiters' cells row 3 (DC3), Hensen's cells (Hec), Claudius cells/outer sulcus cells (CC/OSC), interdental cells (Idc), inner sulcus cells (ISC), Kölliker's organ cells (KO), greater ridge epithelial ridge cells (GER) (including lateral greater epithelial ridge cells (LGER)), and OC90+ cells (OC90), fibroblasts, and other cells of the lateral wall. In some aspects, administration is to the inner ear of the subject. In some aspects, the administration is to the cochlea of the subject. In some aspects, the administration is via a round window membrane injection.

Also provided herein are methods of reducing toxicity associated with expression of the therapeutic polypeptide in an inner ear cell. In some aspects, the inner ear cells are inner ear hair cells, spiral ganglion cells, lateral supporting cells, basilar membrane cells, medial supporting cells, spiral limbus cells, or any combination thereof. In some aspects, expression of the therapeutic polypeptide is reduced, suppressed, or eliminated in inner ear hair cells, spiral ganglion cells, lateral supporting cells, basilar membrane cells, medial supporting cells, spiral limbus cells, or any combination thereof. In some aspects, toxicity due to expression of the therapeutic polypeptide is reduced in inner ear hair cells, spiral ganglion cells, lateral supporting cells, basilar membrane cells, medial supporting cells, spiral limbus cells, or any combination thereof. In some aspects, the therapeutic protein is predominately expressed in inner ear supporting cells. In some aspects, the inner ear supporting cells are selected from one or more of inner phalangeal cells/border cells (IPhC), inner pillar cells (IPC), outer pillar cells (OPC), Deiters' cells rows 1 and 2 (DC1/2), Deiters' cells row 3 (DC3), Hensen's cells (Hec), Claudius cells/outer sulcus cells (CC/OSC), interdental cells (Idc), inner sulcus cells (ISC), Kölliker's organ cells (KO), greater ridge epithelial ridge cells (GER) (including lateral greater epithelial ridge cells (LGER)), and OC90+ cells (OC90), fibroblasts, and other cells of the lateral wall. In some aspects, administration is to the inner ear of the subject. In some aspects, the administration is to the cochlea of the subject. In some aspects, the administration is via a round window membrane injection.

Also provided herein are methods of reducing toxicity associated with expression of the polypeptide in an inner ear cell. In some aspects, the inner ear cells are inner ear hair cells, spiral ganglion cells, lateral supporting cells, basilar membrane cells, medial supporting cells, spiral limbus cells, or any combination thereof. In some aspects, expression of the polypeptide is reduced, suppressed, or eliminated in inner ear hair cells, spiral ganglion cells, lateral supporting cells, basilar membrane cells, medial supporting cells, spiral limbus cells, or any combination thereof. In some aspects, toxicity due to expression of the polypeptide is reduced in inner ear hair cells, spiral ganglion cells, lateral supporting cells, basilar membrane cells, medial supporting cells, spiral limbus cells, or any combination thereof. In some aspects, the protein is predominately expressed in inner ear supporting cells. In some aspects, the inner ear supporting cells are selected from one or more of inner phalangeal cells/border cells (IPhC), inner pillar cells (IPC), outer pillar cells (OPC), Deiters' cells rows 1 and 2 (DC1/2), Deiters' cells row 3 (DC3), Hensen's cells (Hec), Claudius cells/outer sulcus cells (CC/OSC), interdental cells (Idc), inner sulcus cells (ISC), Kölliker's organ cells (KO), greater ridge epithelial ridge cells (GER) (including lateral greater epithelial ridge cells (LGER)), and OC90+ cells (OC90), fibroblasts, and other cells of the lateral wall. In some aspects, administration is to the inner ear of the subject. In some aspects, the administration is to the cochlea of the subject. In some aspects, the administration is via a round window membrane injection.

Also provided herein are methods of treating hearing loss in a subject suffering from or at risk of hearing loss. In some aspects, expression of the therapeutic polypeptide is reduced, suppressed, or eliminated in inner ear hair cells, spiral ganglion cells, lateral supporting cells, basilar membrane cells, medial supporting cells, spiral limbus cells, or any combination thereof. In some aspects, toxicity due to expression of the therapeutic polypeptide is reduced in inner ear hair cells, spiral ganglion cells, lateral supporting cells, basilar membrane cells, medial supporting cells, spiral limbus cells, or any combination thereof. In some aspects, the therapeutic protein is predominately expressed in inner ear supporting cells. In some aspects, the inner ear supporting cells are selected from one or more of inner phalangeal cells/border cells (IPhC), inner pillar cells (IPC), outer pillar cells (OPC), Deiters' cells rows 1 and 2 (DC1/2), Deiters' cells row 3 (DC3), Hensen's cells (Hec), Claudius cells/outer sulcus cells (CC/OSC), interdental cells (Idc), inner sulcus cells (ISC), Kölliker's organ cells (KO), greater ridge epithelial ridge cells (GER) (including lateral greater epithelial ridge cells (LGER)), and OC90+ cells (OC90), fibroblasts, and other cells of the lateral wall.

Also provided herein are methods of treating hearing loss in a subject suffering from or at risk of hearing loss. In some aspects, expression of the polypeptide is reduced, suppressed, or eliminated in inner ear hair cells, spiral ganglion cells, lateral supporting cells, basilar membrane cells, medial supporting cells, spiral limbus cells, or any combination thereof. In some aspects, toxicity due to expression of the polypeptide is reduced in inner ear hair cells, spiral ganglion cells, lateral supporting cells, basilar membrane cells, medial supporting cells, spiral limbus cells, or any combination thereof. In some aspects, the protein is predominately expressed in inner ear supporting cells. In some aspects, the inner ear supporting cells are selected from one or more of inner phalangeal cells/border cells (IPhC), inner pillar cells (IPC), outer pillar cells (OPC), Deiters' cells rows 1 and 2 (DC1/2), Deiters' cells row 3 (DC3), Hensen's cells (Hec), Claudius cells/outer sulcus cells (CC/OSC), interdental cells (Idc), inner sulcus cells (ISC), Kölliker's organ cells (KO), greater ridge epithelial ridge cells (GER) (including lateral greater epithelial ridge cells (LGER)), and OC90+ cells (OC90), fibroblasts, and other cells of the lateral wall.

In some aspects, administration is to the inner ear of the subject. In some aspects, the administration is to the cochlea of the subject. In some aspects, the administration is via a round window membrane injection.

Also provided herein are methods that include administering to an inner ear of a subject a therapeutically effective amount of any of the compositions described herein.

Also provided herein are surgical methods for treatment of hearing loss (e.g., nonsyndromic sensorineural hearing loss or syndromic sensorineural hearing loss). In some aspects, the methods include the steps of: introducing into a cochlea of a subject a first incision at a first incision point; and administering intra-cochlearly a therapeutically effective amount of any of the compositions provided herein. In some aspects, the composition is administered to the subject at the first incision point. In some aspects, the composition is administered to the subject into or through the first incision.

In some aspects of any of the methods described herein, any composition described herein is administered to the subject into or through the cochlea oval window membrane. In some aspects of any of the methods described herein, any of the compositions described herein is administered to the subject into or through the cochlea round window membrane. In some aspects of any of the methods described herein, the composition is administered using a medical device capable of creating a plurality of incisions in the round window membrane. In some aspects, the medical device includes a plurality of micro-needles. In some aspects, the medical device includes a plurality of micro-needles including a generally circular first aspect, where each micro-needle has a diameter of at least about 10 microns. In some aspects, the medical device includes a base and/or a reservoir capable of holding the composition. In some aspects, the medical device includes a plurality of hollow micro-needles individually including a lumen capable of transferring the composition. In some aspects, the medical device includes a means for generating at least a partial vacuum.

In some aspects, technologies of the present disclosure are used to treat subjects with or at risk of hearing loss. In some such aspects, a pathogenic variant causes or is at risk of causing hearing loss.

In some aspects, a subject experiencing hearing loss will be evaluated to determine if and where one or more mutations may exist that may cause hearing loss. In some aspects of any of the methods described herein, the subject or animal is a mammal, in some aspects the mammal is a domestic animal, a farm animal, a zoo animal, a non-human primate, or a human. In some aspects of any of the methods described herein, the animal, subject, or mammal is an adult, a teenager, a juvenile, a child, a toddler, an infant, or a newborn. In some aspects of any of the methods described herein, the animal, subject, or mammal is 1-5, 1-10, 1-20, 1-30, 1-40, 1-50, 1-60, 1-70, 1-80, 1-90, 1-100, 1-110, 2-5, 2-10, 10-20, 20-30, 30-40, 40-50, 50-60, 60-70, 70-80, 80-90, 90-100, 100-110, 10-30, 10-40, 10-50, 10-60, 10-70, 10-80, 10-90, 10-100, 10-110, 20-40, 20-50, 20-60, 20-70, 20-80, 20-90, 20-100, 20-110, 30-50, 30-60, 30-70, 30-80, 30-90, 30-100, 40-60, 40-70, 40-80, 40-90, 40-100, 50-70, 50-80, 50-90, 50-100, 60-80, 60-90, 60-100, 70-90, 70-100, 70-110, 80-100, 80-110, or 90-110 years of age. In some aspects of any of the methods described herein, the subject or mammal is 1, 2, 3, 4, 5, 6, 7, 8, 9, 10, or 11 months of age.

In some aspects of any of the methods described herein, the methods result in improvement in hearing (e.g., any of the metrics for determining improvement in hearing described herein) in a subject in need thereof for at least 10 days, at least 15 days, at least 20 days, at least 25 days, at least 30 days, at least 35 days, at least 40 days, at least 45 days, at least 50 days, at least 55 days, at least 60 days, at least 65 days, at least 70 days, at least 75 days, at least 80 days, at least 85 days, at least 100 days, at least 105 days, at least 110 days, at least 115 days, at least 120 days, at least 5 months, at least 6 months, at least 7 months, at least 8 months, at least 9 months, at least 10 months, at least 11 months, or at least 12 months.

In some aspects a subject (e.g., an animal, e.g., a mammal, e.g., a human) has or is at risk of developing syndromic or nonsyndromic sensorineural hearing loss.

In some aspects, a subject (e.g., an animal, e.g., a mammal, e.g., a human) has been identified as having syndromic or nonsyndromic sensorineural hearing loss.

In some aspects, a subject (e.g., an animal, e.g., a mammal, e.g., a human) has been identified as being at risk of hearing loss (e.g., at risk of being a carrier of a gene mutation). In some such aspects, a subject (e.g., an animal, e.g., a mammal, e.g., a human) may have certain risk factors of hearing loss or risk of hearing loss (e.g., known parental carrier, afflicted sibling, or symptoms of hearing loss). In some such aspects, a subject (e.g., an animal, e.g., a mammal, e.g., a human) has been identified as being a carrier of a mutation in a gene (e.g., via genetic testing) that has not previously been identified ( ). In some such aspects, identified mutations may be novel (i.e., not previously described in the literature), and methods of treatment for a subject suffering from or susceptible to hearing loss will be personalized to the mutation(s) of the particular patient.

In some aspects, successful treatment of syndromic or nonsyndromic sensorineural hearing loss can be determined in a subject using any of the conventional functional hearing tests known in the art. Non-limiting examples of functional hearing tests are various types of audiometric assays (e.g., pure-tone testing, speech testing, test of the middle ear, auditory brainstem response, and otoacoustic emissions).

In some aspects of any method provided herein, two or more doses of any composition described herein are introduced or administered into a cochlea of a subject. Some aspects of any of these methods can include introducing or administering a first dose of a composition into a cochlea of a subject, assessing hearing function of the subject following introduction or administration of a first dose, and administering an additional dose of a composition into the cochlea of the subject found not to have a hearing function within a normal range (e.g., as determined using any test for hearing known in the art).

In some aspects of any method provided herein, the composition can be formulated for intra-cochlear administration. In some aspects of any of the methods described herein, the compositions described herein can be administered via intra-cochlear administration or local administration. In some aspects of any of the methods described herein, the compositions are administered through the use of a medical device (e.g., any of the exemplary medical devices described herein).

In some aspects, intra-cochlear administration can be performed using any of the methods described herein or known in the art. For example, in some aspects, a composition can be administered or introduced into the cochlea using the following surgical technique: first using visualization with a 0 degree, 2.5-mm rigid endoscope, the external auditory canal is cleared and a round knife is used to sharply delineate an approximately 5-mm tympanomeatal flap. The tympanomeatal flap is then elevated and the middle ear is entered posteriorly. The chorda tympani nerve is identified and divided, and a curette is used to remove the scutal bone, exposing the round window membrane. To enhance apical distribution of the administered or introduced composition, a surgical laser may be used to make a small 2-mm fenestration in the oval window to allow for perilymph displacement during trans-round window membrane infusion of the composition. The microinfusion device is then primed and brought into the surgical field. The device is maneuvered to the round window, and the tip is seated within the bony round window overhang to allow for penetration of the membrane by the microneedle(s). The footpedal is engaged to allow for a measured, steady infusion of the composition. The device is then withdrawn and the round window and stapes foot plate are sealed with a gelfoam patch.

In some aspects of any method provided herein, a subject has or is at risk of developing syndromic or nonsyndromic sensorineural hearing loss. In some aspects of any method provided herein, a subject has been previously identified as having a mutation in an inner ear cell target gene, a gene which may be expressed in supporting cells and/or hair cells.

In some aspects of any method provided herein, a subject has been identified as being a carrier of a mutation in an inner ear cell target gene (e.g., via genetic testing). In some aspects of any method provided herein, a subject has been identified as having a mutation in an inner ear cell target gene and has been diagnosed with hearing loss (e.g., nonsyndromic sensorineural hearing loss or syndromic sensorineural hearing loss, e.g., DFNB1, DFNA3). Bart-Pumphrey syndrome, hystrix-like ichthyosis with deafness (HID), palmoplantar keratoderma with deafness, keratitis-ichthyosis-deafness (KID) syndrome, or Vohwinkel syndrome, respectively). In some aspects of any of the methods described herein, the subject has been identified as having hearing loss (e.g., nonsyndromic sensorineural hearing loss or syndromic sensorineural hearing loss). In some aspects, successful treatment of hearing loss (e.g., nonsyndromic sensorineural hearing loss or syndromic sensorineural hearing loss) can be determined in a subject using any of the conventional functional hearing tests known in the art. Non-limiting examples of functional hearing tests include various types of audiometric assays (e.g., pure-tone testing, speech testing, test of the middle ear, auditory brainstem response, and otoacoustic emissions).

In some aspects, a subject cell is in vitro. In some aspects, a subject cell is originally obtained from a subject and is cultured ex vivo. In some aspects, the ex vivo cell is an inner ear cell. In some aspects, the ex vivo cell is an inner phalangeal cells/border cells (IPhC), inner pillar cells (IPC), outer pillar cells (OPC), Deiters' cells rows 1 and 2 (DC1/2), Deiters' cells row 3 (DC3), Hensen's cells (Hec), Claudius cells/outer sulcus cells (CC/OSC), interdental cells (Idc), inner sulcus cells (ISC), Kölliker's organ cells (KO), greater ridge epithelial ridge cells (GER) (including lateral greater epithelial ridge cells (LGER)), and OC90+ cells (OC90), fibroblasts, and other cells of the lateral wall.

In some aspects, a subject cell has previously been determined to have a defective inner ear cell target gene. In some aspects, a subject cell has previously been determined to have a defective hair cell target gene. In some aspects, a subject cell has previously been determined to have a defective supporting cell target gene.

In some aspects of these methods, following treatment e.g., one or two or more administrations of compositions described herein, there is an increase in expression of an active inner ear cell target protein (e.g., a polypeptide (e.g., a therapeutic polypeptide)). In some aspects, an increase in expression of an active inner ear target protein as described herein (e.g., a polypeptide (e.g., a therapeutic polypeptide)) is relative to a control level, e.g., as compared to the level of expression of an inner ear cell target protein prior to introduction of the compositions comprising any construct(s) as described herein.

Methods of detecting expression and/or activity of a target protein (e.g., a polypeptide (e.g., a therapeutic polypeptide)) are known in the art. In some aspects, a level of expression of an inner ear cell target protein can be detected directly (e.g., detecting inner ear cell target protein or target mRNA. Non-limiting examples of techniques that can be used to detect expression and/or activity of a target RNA or protein (e.g., a polypeptide (e.g., a therapeutic polypeptide)) directly include: real-time PCR, Western blotting, immunoprecipitation, immunohistochemistry, mass spectrometry, or immunofluorescence. In some aspects, expression of an inner ear cell target protein can be detected indirectly (e.g., through functional hearing tests).

### Devices, Administration, and Surgical Methods

Provided herein are therapeutic delivery systems for treating hearing loss (e.g., nonsyndromic sensorineural hearing loss or syndromic sensorineural hearing loss). In one aspect, a therapeutic delivery system includes: i) a medical device capable of creating one or a plurality of incisions in a round window membrane of an inner ear of a subject in need thereof, and ii) an effective dose of a composition (e.g., any of the compositions described herein). In some aspects, a medical device includes a plurality of micro-needles.

Also provided herein are surgical methods for treatment of hearing loss (e.g., nonsyndromic sensorineural hearing loss or syndromic sensorineural hearing loss). In some aspects, a method the steps of: introducing into a cochlea of a subject a first incision at a first incision point; and administering intra-cochlearly a therapeutically effective amount of any of the compositions provided herein. In some aspects, a composition is administered to a subject at the first incision point. In some aspects, a composition is administered to a subject into or through the first incision.

In some aspects of any method provided herein, any of the compositions described herein is administered to the subject into or through the cochlea oval window membrane. In some aspects of any method provided herein, any of the compositions described herein is administered to the subject into or through the cochlea round window membrane. In some aspects of any method provided herein, the composition is administered using a medical device capable of creating a plurality of incisions in the round window membrane. In some aspects, a medical device includes a plurality of micro-needles. In some aspects, a medical device includes a plurality of micro-needles including a generally circular first aspect, where each micro-needle has a diameter of at least about 10 microns. In some aspects, a medical device includes a base and/or a reservoir capable of holding a composition. In some aspects, a medical device includes a plurality of hollow micro-needles individually including a lumen capable of transferring a composition. In some aspects, a medical device includes a means for generating at least a partial vacuum.

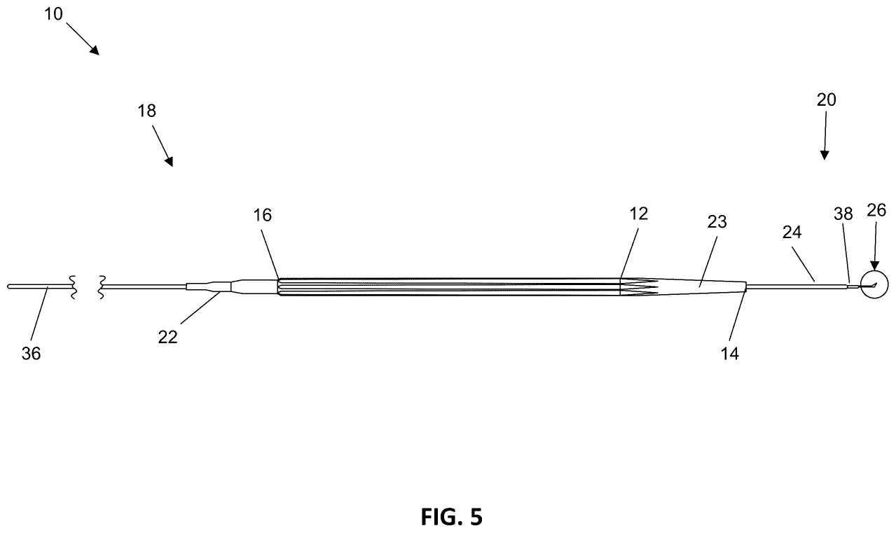

In some aspects, the present disclosure describes a delivery approach that utilizes a minimally invasive, well-accepted surgical technique for accessing the middle ear and/or inner ear through the external auditory canal. The procedure includes opening one of the physical barriers between the middle and inner ear at the oval window, and subsequently using a device disclosed herein, e.g., as shown in FIGS. 5-8 (or microcatheter) to deliver a composition disclosed herein at a controlled flow rate and in a fixed volume, via the round window membrane.

In some aspects, surgical procedures for mammals (e.g., rodents (e.g., mice, rats, hamsters, or rabbits), primates (e.g., NHP (e.g., macaque, chimpanzees, monkeys, or apes) or humans) may include venting to increase AAV vector transduction rates along the length of the cochlea. In some aspects, absence of venting during surgery may result in lower AAV vector cochlear cell transduction rates when compared to AAV vector cochlear cell transduction rates following surgeries performed with venting. In some aspects, venting facilitates transduction rates of about 75-100% of IHCs throughout the cochlea. In some aspects, venting permits IHC transduction rates of about 50-70%, about 60-80%, about 70-90%, or about 80-100% at the base of the cochlea. In some aspects, venting permits IHC transduction rates of about 50-70%, about 60-80%, about 70-90%, or about 80-100% at the apex of the cochlea.

A delivery device described herein may be placed in a sterile field of an operating room and the end of a tubing may be removed from the sterile field and connected to a syringe that has been loaded with a composition disclosed herein (e.g., one or more AAV vectors) and mounted in the pump. After appropriate priming of the system in order to remove any air, a needle may then be passed through the middle ear under visualization (surgical microscope, endoscope, and/or distal tip camera). A needle (or microneedle) may be used to puncture the RWM. The needle may be inserted until a stopper contacts the RWM. The device may then be held in that position while a composition disclosed herein is delivered at a controlled flow rate to the inner ear, for a selected duration of time. In some aspects, the flow rate (or infusion rate) may include a rate of about 30 μL/min, or from about 25 μL/min to about 35 μL/min, or from about 20 μL/min to about 40 μL/min, or from about 20 μL/min to about 70 μL/min, or from about 20 μL/min to about 90 μL/min, or from about 20 μL/min to about 100 μL/min. In some aspects, the flow rate is about 20 μL/min, about 30 μL/min, about 40 μL/min, about 50 μL/min, about 60 μL/min, about 70 μL/min, about 80 μL/min, about 90 μL/min or about 100 μL/min. In some aspects, the selected duration of time (that is, the time during which a composition disclosed herein is flowing) may be about 3 minutes, or from about 2.5 minutes to about 3.5 minutes, or from about 2 minutes to about 4 minutes, or from about 1.5 minutes to about 4.5 minutes, or from about 1 minute to about 5 minutes. In some aspects, the total volume of a composition disclosed herein that flows to the inner ear may be about 0.09 mL, or from about 0.08 mL to about 0.10 mL, or from about 0.07 mL to about 0.11 mL. In some aspects, the total volume of a composition disclosed herein equates to from about 40% to about 50% of the volume of the inner ear.

Once the delivery has been completed, the device may be removed. In some aspects, a device described herein, may be configured as a single-use disposable product. In other aspects, a device described herein may be configured as a multi-use, sterilizable product, for example, with a replaceable and/or sterilizable needle sub-assembly. Single use devices may be appropriately discarded (for example, in a biohazard sharps container) after administration is complete.

In some aspects, a composition disclosed herein comprises one or a plurality of rAAV constructs. In some aspects, when more than one rAAV construct is included in the composition, the rAAV constructs are each different. In some aspects, an rAAV construct comprises an anti-VEGF coding region, e.g., as described herein. In some aspects, a composition comprises an rAAV particle comprising an AAV construct described herein. In some aspects, the rAAV particle is encapsidated by an Anc80 capsid (e.g., an Anc80L65 capsid). In some aspects, the Anc80 capsid comprises a polypeptide of SEQ ID NO: 44.

In some aspects, a composition disclosed herein can be administered to a subject with a surgical procedure. In some aspects, administration, e.g., via a surgical procedure, comprises injecting a composition disclosed herein via a delivery device as described herein into the inner ear. In some aspects, a surgical procedure disclosed herein comprises performing a transcanal tympanotomy; performing a laser-assisted micro-stapedotomy; and injecting a composition disclosed herein via a delivery device as described herein into the inner ear.

In some aspects, a surgical procedure comprises performing a transcanal tympanotomy; performing a laser-assisted micro-stapedotomy; injecting a composition disclosed herein via a delivery device as described herein into the inner ear; applying sealant around the round window and/or an oval window of the subject; and lowering a tympanomeatal flap of the subject to the anatomical position.

In some aspects, a surgical procedure comprises performing a transcanal tympanotomy; preparing a round window of the subject; performing a laser-assisted micro-stapedotomy; preparing both a delivery device as described herein and a composition disclosed herein for delivery to the inner ear; injecting a composition disclosed herein via the delivery device into the inner ear; applying sealant around the round window and/or an oval window of the subject; and lowering a tympanomeatal flap of the subject to the anatomical position.

In some aspects, performing a laser-assisted micro-stapedotomy includes using a KTP otologic laser and/or a CO2 otologic laser.

As another example, a composition disclosed herein is administered using a device and/or system specifically designed for intracochlear route of administration. In some aspects, design elements of a device described herein may include: maintenance of sterility of injected fluid; minimization of air bubbles introduced to the inner ear; ability to precisely deliver small volumes at a controlled rate; delivery through the external auditory canal by the surgeon; minimization of damage to the round window membrane (RWM), or to inner ear, e.g., cochlear structures beyond the RWM; and/or minimization of injected fluid leaking back out through the RWM.

The devices, systems, and methods provided herein also describe the potential for delivering a composition safely and efficiently into the inner ear, in order to treat conditions and disorders that would benefit from delivery of a composition disclosed herein to the inner ear, including, but not limited to, hearing disorders, e.g., as described herein. As another example, by placing a vent in the stapes footplate and injecting through the RWM, a composition disclosed herein is dispersed throughout the cochlea with minimal dilution at the site of action. The development of the described devices allows the surgical administration procedure to be performed through the external auditory canal in humans. The described devices can be removed from the ear following infusion of an amount of fluid into the perilymph of the cochlea. In subjects, the device may be advanced through the external auditory canal, either under surgical microscopic control or along with an endoscope.

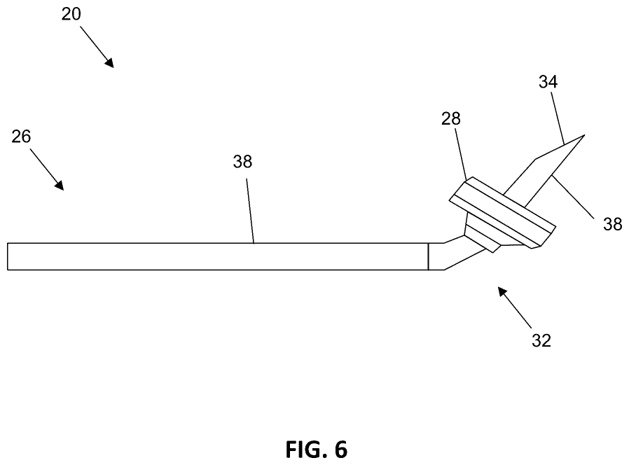

An exemplary device for use in any of the methods disclosed herein is described in FIGS. 5-8. FIG. 5 illustrates an exemplary device 10 for delivering fluid to an inner ear. Device 10 includes a knurled handle 12, and a distal handle adhesive 14 (for example, an epoxy such as Loctite 4014) that couples to a telescoping hypotube needle support 24. The knurled handle 12 (or handle portion) may include kurling features and/or grooves to enhance the grip. The knurled handle 12 (or handle portion) may be from about 5 mm to about 15 mm thick or from about 5 mm to about 12 mm thick, or from about 6 mm to about 10 mm thick, or from about 6 mm to about 9 mm thick, or from about 7 mm to about 8 mm thick. The knurled handle 12 (or handle portion) may be hollow such that fluid may pass through the device 10 during use. The device 10 may also include a proximal handle adhesive 16 at a proximal end 18 of the knurled handle 12, a needle sub-assembly 26 (shown in FIG. 6) with stopper 28 (shown in FIG. 34) at a distal end 20 of the device 10, and a strain relief feature 22. Strain relief feature 22 may be composed of a Santoprene material, a Pebax material, a polyurethane material, a silicone material, a nylon material, and/or a thermoplastic elastomer. The telescoping hypotube needle support 24 surrounds and supports a bent needle 38 (shown in FIG. 6) disposed therewithin.

Referring still to FIG. 5, the stopper 28 may be composed of a thermoplastic material or plastic polymer (such as a UV-cured polymer), as well as other suitable materials, and may be used to prevent the bent needle 38 from being inserted too far into the ear canal (for example, to prevent insertion of bent needle 38 into the lateral wall or other inner ear structure). Device 10 also may include a tapered portion 23 disposed between the knurled handle 12 and the distal handle adhesive 14 that is coupled to the telescoping hypotube needle support 24. The knurled handle 12 (or handle portion) may include the tapered portion 23 at the distal end of the handle portion 12. Device 10 may also include tubing 36 fluidly connected to the proximal end 16 the device 10 and acts as a fluid inlet line connecting the device to upstream components (for example, a pump, a syringe, and/or upstream components which, in some aspects, may be coupled to a control system and/or power supply (not shown)). In some aspects, the bent needle 38 (shown in FIG. 6) extends from the distal end 20, through the telescoping hypotube needle support 24, through the tapered portion 23, through the knurled handle 12, and through the strain relief feature 22 and fluidly connects directly to the tubing 36. In other aspects, the bent needle 38 fluidly connects with the hollow interior of the knurled handle (for example, via the telescoping hypotube needle support 24) which in turn fluidly connects at a proximal end 16 with tubing 36. In aspects where the bent needle 38 does not extend all the way through the interior of the device 10, the contact area (for example, between overlapping nested hypotubes 42), the tolerances, and/or sealants between interfacing components must be sufficient to prevent therapeutic fluid from leaking out of the device 10 (which operates at a relatively low pressure (for example, from about 1 Pascal to about 50 Pa, or from about 2 Pa to about 20 Pa, or from about 3 Pa to about 10 Pa)).

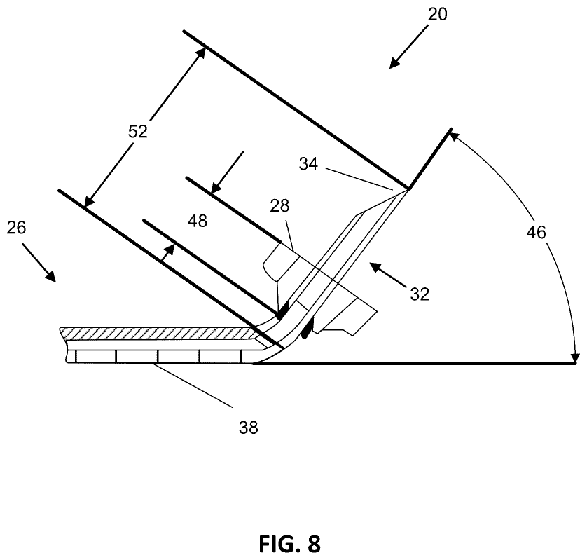

FIG. 6 illustrates a sideview of the bent needle sub-assembly 26, according to aspects of the present disclosed aspects. Bent needle sub-assembly 26 includes a needle 38 that has a bent portion 32. Bent needle sub-assembly 26 may also include a stopper 28 coupled to the bent portion 32. The bent portion 32 includes an angled tip 34 at the distal end 20 of the device 10 for piercing a membrane of the ear (for example, the RWM). The needle 38, bent portion 32, and angled top 34 are hollow such that fluid may flow therethrough. The angle 46 (as shown in FIG. 8) of the bent portion 32 may vary. A stopper 28 geometry may be cylindrical, disk-shaped, annulus-shaped, dome-shaped, and/or other suitable shapes. Stopper 28 may be molded into place onto bent portion 32. For example, stopper 28 may be positioned concentrically around the bent portion 32 using adhesives or compression fitting. Examples of adhesives include an UV cure adhesive (such as Dymax 203A-CTH-F-T), elastomer adhesives, thermoset adhesives (such as epoxy or polyurethane), or emulsion adhesives (such as polyvinyl acetate). Stopper 28 fits concentrically around the bent portion 32 such that angled tip 34 is inserted into the ear at a desired insertion depth. The bent needle 38 may be formed from a straight needle using incremental forming, as well as other suitable techniques.

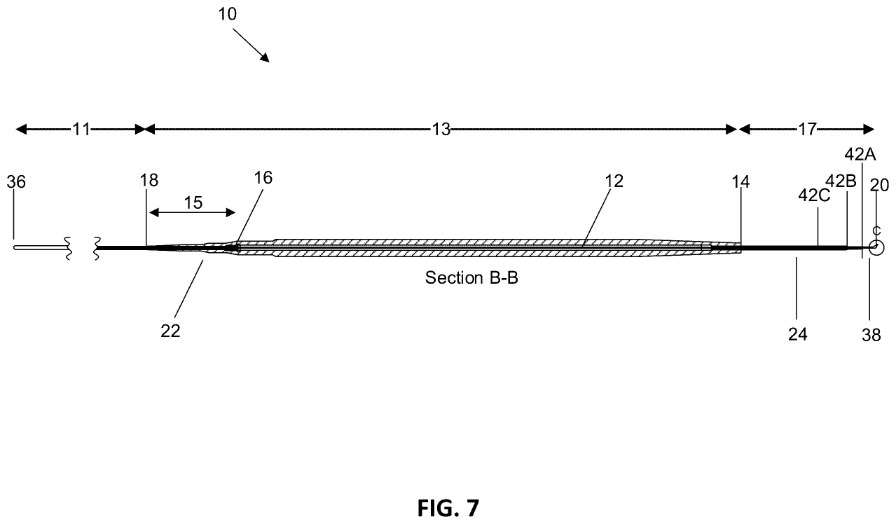

FIG. 7 illustrates a perspective view of exemplary device 10 for delivering fluid to an inner ear. Tubing 36 may be from about 1300 mm in length (dimension 11 in FIG. 7) to about 1600 mm, or from about 1400 mm to about 1500 mm, or from about 1430 mm to about 1450 mm. Strain release feature 22 may be from about 25 mm to about 30 mm in length (dimension 15 in FIG. 7), or from about 20 mm to about 35 mm in length. Handle 12 may be about 155.4 mm in length (dimension 13 in FIG. 7), or from about 150 mm to about 160 mm, or from about 140 mm to about 170 mm. The telescoping hypotube needle support 24 may have two or more nested hypotubes, for example three nested hypotubes 42A, 42B, and 42C, or four nested hypotubes 42A, 42B, 42C, and 42D. The total length of hypotubes 42A, 42B, 42C and tip assembly 26 (dimension 17 in FIG. 7) may be from about 25 mm to about 45 mm, or from about 30 mm to about 40 mm, or about 35 mm. In addition, telescoping hypotube needle support 24 may have a length of about 36 mm, or from about 25 mm to about 45 mm, or form about 30 mm to about 40 mm. The three nested hypotubes 42A, 42B, and 42C each may have a length of 3.5 mm, 8.0 mm, and 19.8 mm, respectively, plus or minus about 20%. The inner-most nested hypotube (or most narrow portion) of the telescoping hypotube needle support 24 may be concentrically disposed around needle 38.

FIG. 8 illustrates a perspective view of bent needle sub-assembly 26 coupled to the distal end 20 of device 10, according to aspects of the present disclosed aspects. As shown in FIG. 8, bent needle sub-assembly 26 may include a needle 38 coupled to a bent portion 32. In other aspects, the bent needle 38 may be a single needle (for example, a straight needle that is then bent such that it includes the desired angle 46). Needle 38 may be a 33-gauge needle, or may include a gauge from about 32 to about 34, or from about 31 to 35. At finer gauges, care must be taken to ensure tubing 36 is not kinked or damaged. Needle 38 may be attached to handle 12 for safe and accurate placement of needle 38 into the inner ear. As shown in FIG. 8, bent needle sub-assembly 26 may also include a stopper 28 disposed around bent portion 32. FIG. 8 also shows that bent portion 32 may include an angled tip 34 for piercing a membrane of the ear (for example, the RWM). Stopper 28 may have a height 48 of about 0.5 mm, or from about 0.4 mm to about 0.6 mm, or from about 0.3 mm to about 0.7 mm. Bent portion 32 may have a length 52 of about 1.45 mm, or from about 1.35 mm to about 1.55 mm, or from about 1.2 mm to about 1.7 mm. In other aspects, the bent portion 32 may have a length greater than 2.0 mm such that the distance between the distal end of the stopper 28 and the distal end of the angled tip 34 is from about 0.5 mm to about 1.7 mm, or from about 0.6 mm to about 1.5 mm, or from about 0.7 mm to about 1.3 mm, or from about 0.8 mm to about 1.2 mm. FIG. 8 shows that stopper 28 may have a geometry that is cylindrical, disk-shaped, and/or dome-shaped. A person of ordinary skill will appreciate that other geometries could be used.

### Evaluating Hearing Loss and Recovery

In some aspects, hearing function is determined using auditory brainstem response measurements (ABR). In some aspects, hearing is tested by measuring distortion product optoacoustic emissions (DPOAEs). In some such aspects, measurements are taken from one or both ears of a subject. In some such aspects, recordings are compared to prior recordings for the same subject and/or known thresholds on such response measurements used to define, e.g., hearing loss versus acceptable hearing ranges to be defined as normal hearing. In some aspects, a subject has ABR and/or DPOAE measurements recorded prior to receiving any treatment. In some aspects, a subject treated with one or more technologies described herein will have improvements on ABR and/or DPOAE measurements after treatment as compared to before treatment. In some aspects, ABR and/or DPOAE measurements are taken after treatment is administered and at regular follow-up intervals post-treatment.

In some aspects, hearing function is determined using speech pattern recognition or is determined by a speech therapist. In some aspects, hearing function is determined by pure tone testing. In some aspects, hearing function is determined by bone conduction testing. In some aspects, hearing function is determined by acoustic reflex testing. In some aspects hearing function is determined by tympanometry. In some aspects, hearing function is determined by any combination of hearing analysis known in the art. In some such aspects, measurements are taken holistically, and/or from one or both ears of a subject. In some such aspects, recordings and/or professional analysis are compared to prior recordings and/or analysis for the same subject and/or known thresholds on such response measurements used to define, e.g., hearing loss versus acceptable hearing ranges to be defined as normal hearing. In some aspects, a subject has speech pattern recognition, pure tone testing, bone conduction testing, acoustic reflex testing and/or tympanometry measurements and/or analysis conducted prior to receiving any treatment. In some aspects a subject treated with one or more technologies described herein will have improvements on speech pattern recognition, pure tone testing, bone conduction testing, acoustic reflex testing and/or tympanometry measurements after treatment as compared to before treatment. In some aspects, speech pattern recognition, pure tone testing, bone conduction testing, acoustic reflex testing and/or tympanometry measurements are taken after treatment is administered and at regular follow-up intervals post-treatment.

### Production Methods

AAV systems are generally well known in the art (see, e.g., Kelleher and Vos, Biotechniques, 17(6):1110-17 (1994); Cotton et al., P.N.A.S. U.S.A., 89(13):6094-98 (1992); Curiel, Nat Immun, 13(2-3):141-64 (1994); Muzyczka, Curr Top Microbiol Immunol, 158:97-129 (1992); and Asokan A, et al., Mol. Ther., 20(4):699-708 (2012), each of which is incorporated in its entirety herein by reference). Methods for generating and using AAV constructs are described, for example, in U.S. Pat. Nos. 5,139,941, 4,797,368 and PCT filing application US2019/060328, each of which is incorporated in its entirety herein by reference.

Methods for obtaining viral constructs are known in the art. For example, to produce AAV constructs, the methods typically involve culturing a host cell which contains a nucleic acid sequence encoding an AAV capsid protein or fragment thereof; a functional rep gene; a recombinant AAV construct composed of AAV inverted terminal repeats (ITRs) and a coding sequence; and/or sufficient helper functions to permit packaging of the recombinant AAV construct into the AAV capsid proteins.

In some aspects, components to be cultured in a host cell to package an AAV construct in an AAV capsid may be provided to the host cell in trans. Alternatively, any one or more components (e.g., recombinant AAV construct, rep sequences, cap sequences, and/or helper functions) may be provided by a stable host cell that has been engineered to contain one or more such components using methods known to those of skill in the art. In some aspects, such a stable host cell contains such component(s) under the control of an inducible promoter. In some aspects, such component(s) may be under the control of a constitutive promoter. In some aspects, a selected stable host cell may contain selected component(s) under the control of a constitutive promoter and other selected component(s) under the control of one or more inducible promoters. For example, a stable host cell may be generated that is derived from HEK293 cells (which contain E1 helper functions under the control of a constitutive promoter), but that contain the rep and/or cap proteins under the control of inducible promoters. Other stable host cells may be generated by one of skill in the art using routine methods.

Recombinant AAV construct, rep sequences, cap sequences, and helper functions required for producing an AAV of the disclosure may be delivered to a packaging host cell using any appropriate genetic element (e.g., construct). A selected genetic element may be delivered by any suitable method known in the art, e.g., to those with skill in nucleic acid manipulation and include genetic engineering, recombinant engineering, and synthetic techniques (see, e.g., Sambrook et al., Molecular Cloning: A Laboratory Manual, Cold Spring Harbor Press, Cold Spring Harbor, N.Y., which is incorporated in its entirety herein by reference). Similarly, methods of generating AAV particles are well known and any suitable method can be used with the present disclosure (see, e.g., K. Fisher et al., J. Virol., 70:520-532 (1993) and U.S. Pat. No. 5,478,745, which are incorporated in their entirety herein by reference).

In some aspects, recombinant AAVs may be produced using a triple transfection method (e.g., as described in U.S. Pat. No. 6,001,650, which is incorporated in its entirety herein by reference). In some aspects, recombinant AAVs are produced by transfecting a host cell with a recombinant AAV construct (comprising a coding sequence) to be packaged into AAV particles, an AAV helper function construct, and an accessory function construct. An AAV helper function construct encodes “AAV helper function” sequences (i.e., rep and cap), which function in trans for productive AAV replication and encapsidation. In some aspects, the AAV helper function construct supports efficient AAV construct production without generating any detectable wild-type AAV particles (i.e., AAV particles containing functional rep and cap genes). Non-limiting examples of constructs suitable for use with the present disclosure include pHLP19 (see, e.g., U.S. Pat. No. 6,001,650, which is incorporated in its entirety herein by reference) and pRep6cap6 construct (see, e.g., U.S. Pat. No. 6,156,303, which is incorporated in its entirety herein by reference). An accessory function construct encodes nucleotide sequences for non-AAV derived viral and/or cellular functions upon which AAV is dependent for replication (i.e., “accessory functions”). Accessory functions may include those functions required for AAV replication, including, without limitation, those moieties involved in activation of AAV gene transcription, stage specific AAV mRNA splicing, AAV DNA replication, synthesis of cap expression products, and AAV capsid assembly. Viral-based accessory functions can be derived from any known helper viruses such as adenovirus, herpesvirus (other than herpes simplex virus type-1), and vaccinia virus.

Additional methods for generating and isolating AAV viral constructs suitable for delivery to a subject are described in, e.g., U.S. Pat. Nos. 7,790,449; 7,282,199; WO 2003/042397; WO 2005/033321, WO 2006/110689; and U.S. Pat. No. 7,588,772, each of which is incorporated in its entirety herein by reference. In one system, a producer cell line is transiently transfected with a construct that encodes a coding sequence flanked by ITRs and a construct(s) that encodes rep and cap. In another system, a packaging cell line that stably supplies rep and cap is transiently transfected with a construct encoding a coding sequence flanked by ITRs. In each of these systems, AAV particles are produced in response to infection with helper adenovirus or herpesvirus, and AAVs are separated from contaminating virus. Other systems do not require infection with helper virus to recover the virus particles—helper functions (i.e., adenovirus E1, E2a, VA, and E4 or herpesvirus UL5, UL8, UL52, and UL29, and herpesvirus polymerase) are also supplied, in trans, by the system. In such systems, helper functions can be supplied by transient transfection of the cells with constructs that encode the helper functions, or the cells can be engineered to stably contain genes encoding the helper functions, the expression of which can be controlled at the transcriptional or posttranscriptional level.

In some aspects, viral construct titers post-purification are determined. In some aspects, titers are determined using quantitative PCR. In certain aspects, a TaqMan probe specific to a construct is utilized to determine construct levels. In certain aspects, the TaqMan probe is represented by SEQ ID NO: 58, while forward and reverse amplifying primers are exemplified by SEQ ID NO: 59 and 60 respectively.
Exemplary Taqman probe for quantification of constructs (SEQ ID NO: 58) /56-FAM/TCTGGCTCA/ZEN/CCGTCCTCTTCATTT/3IABkFQ)/
Exemplary forward qPCR primer for quantification of constructs (SEQ ID NO: 59) CAAACACTCCACCAGCATTG
Exemplary reverse qPCR primer for quantification of constructs (SEQ ID NO: 60) CAGCCACAACGAGGATCATA

As described herein, in some aspects, a viral construct of the present disclosure is an adeno-associated virus (AAV) construct. Several AAV serotypes have been characterized, including AAV1, AAV2, AAV3 (e.g., AAV3B), AAV4, AAV5, AAV6, AAV7, AAV8, AAV9, AAV10, AAV11, and AAV Anc80, as well as variants thereof. In some aspects, an AAV particle is an AAV2/6, AAV2/8, AAV2/9, or AAV2/Anc80 particle (e.g., with AAV6, AAV8, AAV9, or Anc80 capsid (e.g., an Anc80L65 capsid) and construct with AAV2 ITR). Other AAV particles and constructs are described in, e.g., Sharma et al., Brain Res Bull. 2010 Feb. 15; 81(2-3): 273, which is incorporated in its entirety herein by reference. Generally, any AAV serotype may be used to deliver a coding sequence described herein. However, the serotypes have different tropisms, e.g., they preferentially infect different tissues. In some aspects, an AAV construct is a self-complementary AAV construct.

The present disclosure provides, among other things, methods of making AAV-based constructs. In some aspects, such methods include use of host cells. In some aspects, a host cell is a mammalian cell. A host cell may be used as a recipient of an AAV helper construct, an AAV minigene plasmid, an accessory function construct, and/or other transfer DNA associated with the production of recombinant AAVs. The term includes the progeny of an original cell that has been transfected. Thus, a “host cell” as used herein may refer to a cell that has been transfected with an exogenous DNA sequence. It is understood that the progeny of a single parental cell may not necessarily be completely identical in morphology or in genomic or total DNA complement as the original parent, due to natural, accidental, or deliberate mutation.

Additional methods for generating and isolating AAV particles suitable for delivery to a subject are described in, e.g., U.S. Pat. Nos. 7,790,449; 7,282,199; WO 2003/042397; WO 2005/033321, WO 2006/110689; and U.S. Pat. No. 7,588,772, each of which is incorporated in its entirety herein by reference. In one system, a producer cell line is transiently transfected with a construct that encodes a coding sequence flanked by ITRs and a construct(s) that encodes rep and cap. In another system, a packaging cell line that stably supplies rep and cap is transiently transfected with a construct encoding a coding sequence flanked by ITRs. In each of these systems, AAV particles are produced in response to infection with helper adenovirus or herpesvirus, and AAV particles are separated from contaminating virus. Other systems do not require infection with helper virus to recover the AAV particles-—the helper functions (i.e., adenovirus E1, E2a, VA, and E4 or herpesvirus UL5, UL8, UL52, and UL29, and herpesvirus polymerase) are also supplied, in trans, by the system. In such systems, helper functions can be supplied by transient transfection of the cells with constructs that encode the helper functions, or the cells can be engineered to stably contain genes encoding the helper functions, the expression of which can be controlled at the transcriptional or posttranscriptional level.

In yet another system, a coding sequence flanked by ITRs and rep/cap genes are introduced into insect host cells by infection with baculovirus-based constructs. Such production systems are known in the art (see generally, e.g., Zhang et al., 2009, Human Gene Therapy 20:922-929, which is incorporated in its entirety herein by reference). Methods of making and using these and other AAV production systems are also described in U.S. Pat. Nos. 5,139,941; 5,741,683; 6,057,152; 6,204,059; 6,268,213; 6,491,907; 6,660,514; 6,951,753; 7,094,604; 7,172,893; 7,201,898; 7,229,823; and 7,439,065, each of which is incorporated in its entirety herein by reference.

### EXAMPLES

The disclosure is further described in detail by reference to the following experimental examples. These examples are provided for purposes of illustration only, and are not intended to be limiting unless otherwise specified. Thus, the disclosure should in no way be construed as being limited to the following examples, but rather should be construed to encompass any and all variations that become evident as a result of the teaching provided herein.

It is believed that one or ordinary skill in the art can, using the preceding description and following Examples, as well as what is known in the art, make and utilize technologies of the present disclosure.

### Example 1: Construction of Viral Constructs Comprising a Polypeptide or Therapeutic Polypeptide

This example provides a description of generating a viral construct as described herein. A recombinant AAV (rAAV) particle was generated by transfection with an adenovirus-free method as used by Xiao et al., J Virol. 73(5):3994-4003, 1999, which is incorporated in its entirety herein by reference. The cis plasmids with AAV ITRs, the trans plasmid with AAV Rep and Cap genes, and a helper plasmid with an essential region from an adenovirus genome were co-transfected in HEK293 cells. The rAAV construct expressed human connexin 26 under a single construct strategy using the constructs described. AAV Anc80 capsid was prepared to encapsulate a unique rAAV connexin 26 protein encoding construct.

Those of ordinary skill in the art will readily understand that similar constructs can be made in accordance with this example. For instance, rAAV constructs that express mammalian, primate, or human connexin 26 under single, dual, or multi construct strategies can be generated. AAV serotypes 1, 2, 3, 4, 5, 6, 7, 8, 9, rh8, rh10, rh39, rh43, and Anc80 can each be prepared to encapsulate four sets of connexin 26 constructs to test (i) a concatemerization-transplicing strategy, (ii) a hybrid intronic-homologous recombination-transplicing strategy, (iii) an exonic homologous recombination strategy, as summarized by Pryadkina et al., Meth. Clin. Devel. 2:15009, 2015, which is incorporated in its entirety herein by reference, and (iv) a single construct strategy. In some aspects, a recombinant AAV (rAAV) particle is generated by transfection with an adenovirus-free method as used by Xiao et al., J Virol. 73(5):3994-4003, 1999, which is incorporated in its entirety herein by reference.

### Example 2: Generating and Purifying Viral Particles

This example provides a description of purification of a viral construct. A recombinant AAV (rAAV) is produced using a triple transfection protocol and purified. The fractions are analyzed by dot blot to determine those containing rAAV genomes. The viral genome number (vg) of each preparation is determined by a quantitative real-time PCR-based titration method using primers and probe corresponding to the ITR region of the AAV construct genome (Bartoli et al., Gene. Ther. 13:20-28, 2006, which is incorporated in its entirety herein by reference).

In some aspects of this example, a recombinant AAV (rAAV) was produced using a standard triple transfection protocol and purified by two sequential cesium chloride (CsCl) density gradients, as described by Pryadkina et al., Mol. Ther. 2:15009, 2015, which is incorporated in its entirety herein by reference. At the end of second centrifugation, 11 fractions of 500 μl were recovered from the CsCl density gradient tube and purified through dialysis in 1×PBS. The fractions were analyzed by dot blot to determine those containing rAAV genomes. The viral genome number (vg) of each preparation was determined by a quantitative real-time PCR-based titration method using primers and probe corresponding to the ITR region of the AAV construct genome (Bartoli et al., Gene. Ther. 13:20-28, 2006, which is incorporated in its entirety herein by reference).

Those of ordinary skill in the art will readily understand that similar production and purifying processes can be conducted in accordance with this example. For instance, rAAV particles may be purified using various column chromatography methods known in the art, and/or viral genomes may be quantified using alternative primer sets.

### Example 3: Formulation of Viral Particles

This example relates to the preparation of compositions comprising rAAV particles, and a physiologically acceptable solution. An rAAV particle was produced and purified to a titer of 1.2×10¹³ vg/mL and was then prepared at dilutions of 6×10⁴, 1.3×10⁵, 1.8×10⁵, 4.5×10⁹, and 1.3×10¹⁰ vg/mL in a physiologically acceptable solution (e.g., commercially available 1×PBS with pluronic acid F68, prepared to a final concentration of: 8.10 mM Sodium Phosphate Dibasic, 1.5 mM Monopotassium Phosphate, 2.7 mM Potassium Chloride, 172 mM Sodium Chloride, and 0.001% Pluronic Acid F68).

In alternative aspects, an rAAV is produced and purified to a known concentration (e.g., a titer of approximately 1×10¹³ vg/mL) and is then prepared at desired concentrations (e.g., dilutions of 6×10 ⁴, 1.3×10⁵, 1.8×10⁵, 4.5×10⁹, and 1.3×10¹⁰ vg/mL) in a physiologically acceptable buffer (e.g., commercially available 1×PBS with pluronic acid F68, prepared to a final concentration of: 8.10 mM Sodium Phosphate Dibasic, 1.5 mM Monopotassium Phosphate, 2.7 mM Potassium Chloride, 172 mM Sodium Chloride, and 0.001% Pluronic Acid F68; or e.g., artificial perilymph comprising NaCl, 120 mM; KCl, 3.5 mM; CaCl2, 1.5 mM; glucose, 5.5 mM; HEPES, 20 mM. which is titrated with NaOH to adjust its pH to 7.5 (total Na⁺ concentration of 130 mM) as described in Chen et al., J Controlled Rel. 110:1-19, 2005, which is incorporated in its entirety herein by reference). Those of ordinary skill in the art will readily understand that alternative formulations can be prepared in accordance with this example. For instance, rAAV particles may be purified to an alternative titer, prepared at alternative dilutions, and suspended in alternative suitable solutions.

### Example 4: Device Description

This example relates to a device suitable for the delivery of rAAV particles to the inner ear. A composition comprising rAAV particles is delivered to the cochlea of a subject using a specialized microcatheter designed for consistent and safe penetration of the round window membrane (RWM). The microcatheter is shaped such that the surgeon performing the delivery procedure can enter the middle ear cavity via the external auditory canal and contact the end of the microcatheter with the RWM. The distal end of the microcatheter may include at least one microneedle with a diameter from about 10 microns to about 1,000 microns, which produces perforations in the RWM that are sufficient to allow a construct as described (e.g., an rAAV construct) to enter the cochlear perilymph of the scala tympani at a rate which does not damage the inner ear (e.g., a physiologically acceptable rate, e.g., a rate of approximately 30 μL/min to approximately 90 μL/min), but small enough to heal without surgical repair. The remaining portion of the microcatheter, proximal to the microneedle(s), is loaded with the rAAV/artificial perilymph formulation at a defined titer (e.g., approximately 1×10¹² to 5×10¹³ vg/mL). The proximal end of the microcatheter is connected to a micromanipulator that allows for precise, low volume infusions of approximately 30 μL to approximately 100 μL.

### Example 5: In-Vitro Demonstration of GJB2 mRNA and Connexin 26 Protein Production (Anti-FLAG Antibody)

This example relates to the introduction, regulation, and expression analysis of rAAV constructs expressing a supporting cell protein (e.g., a hGJB2 gene) in mammalian cells grown in vitro or ex vivo.

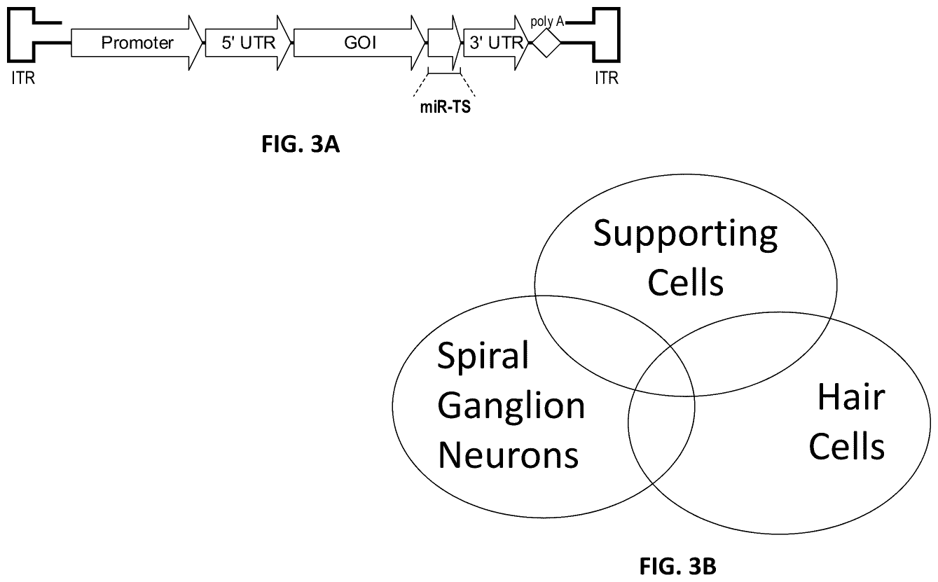

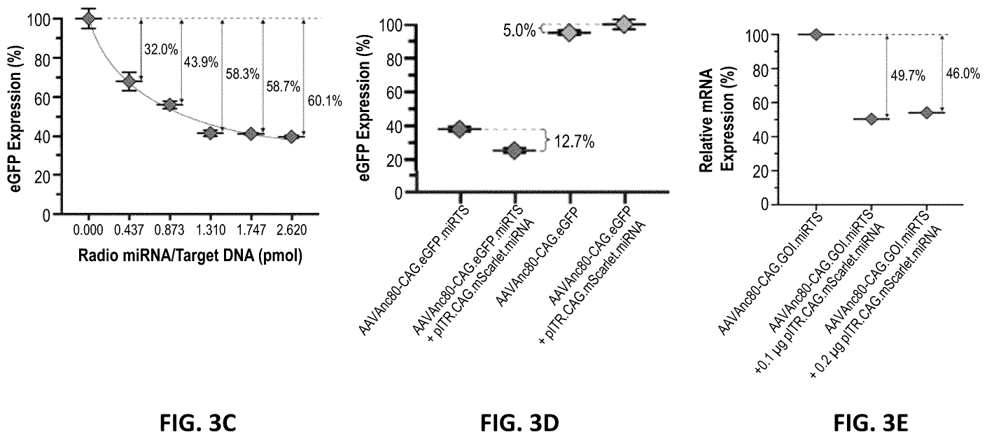

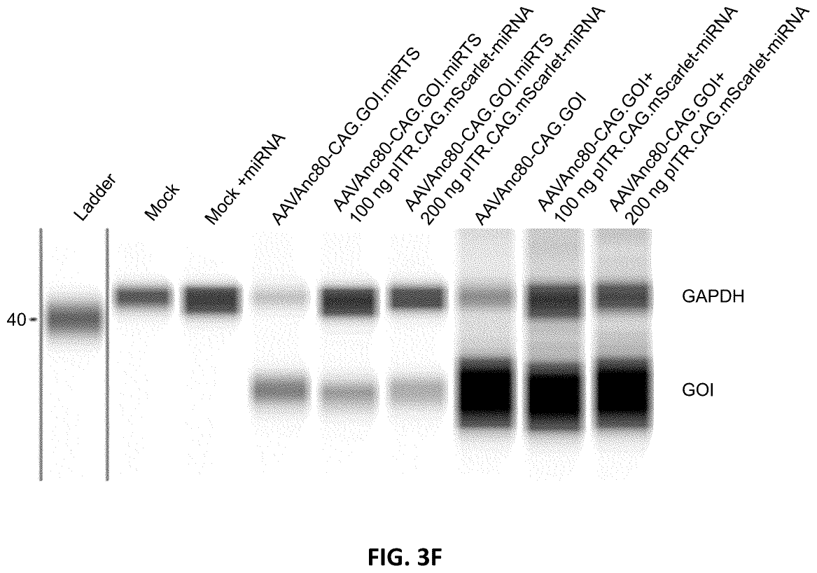

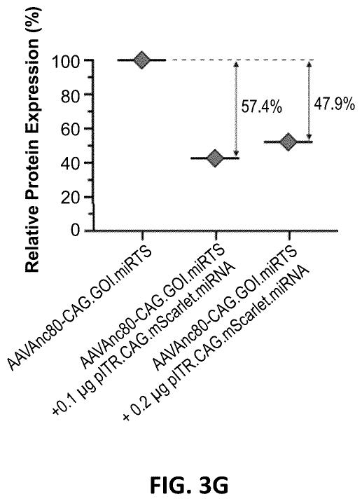

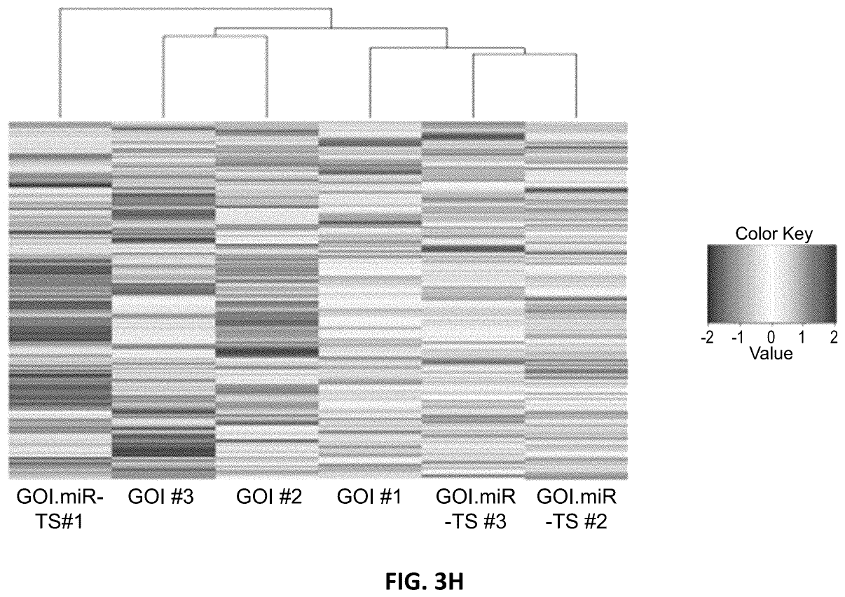

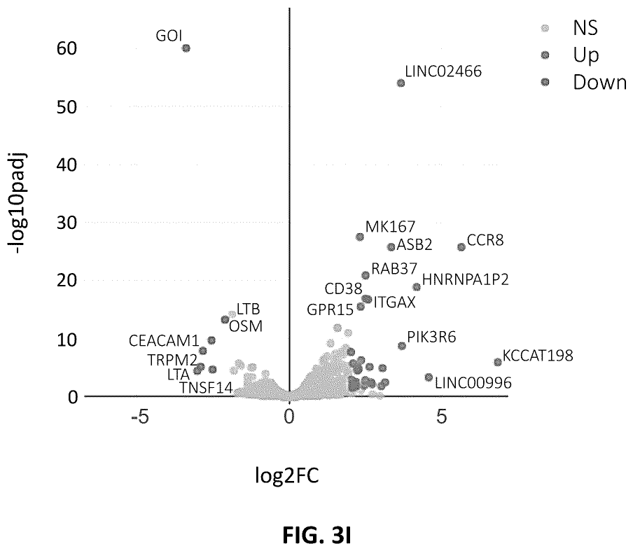

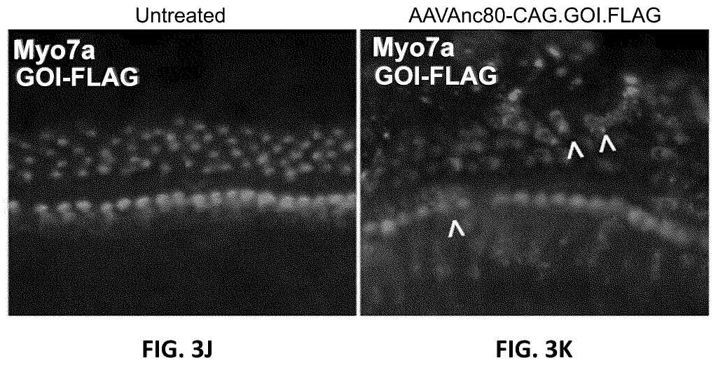

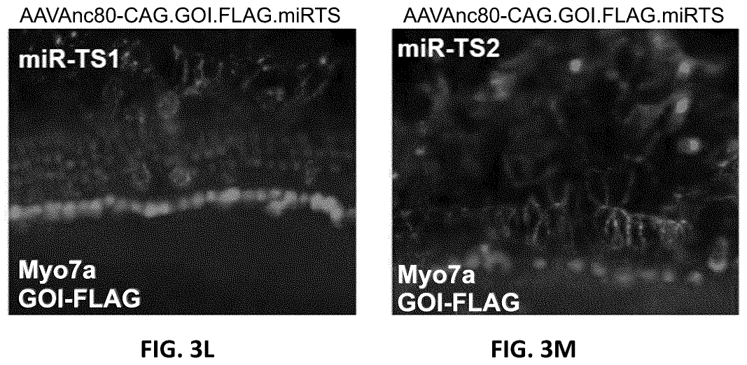

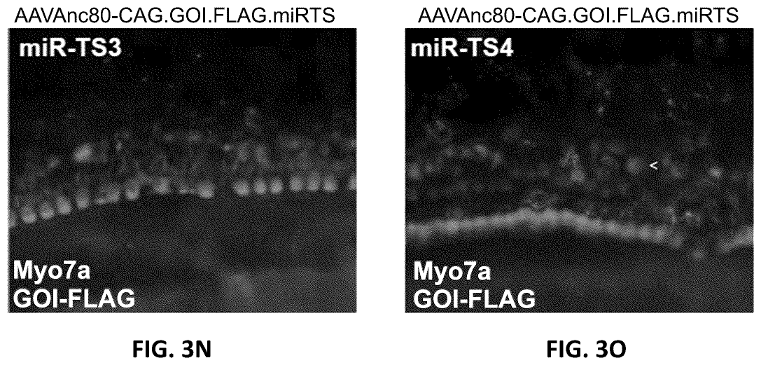

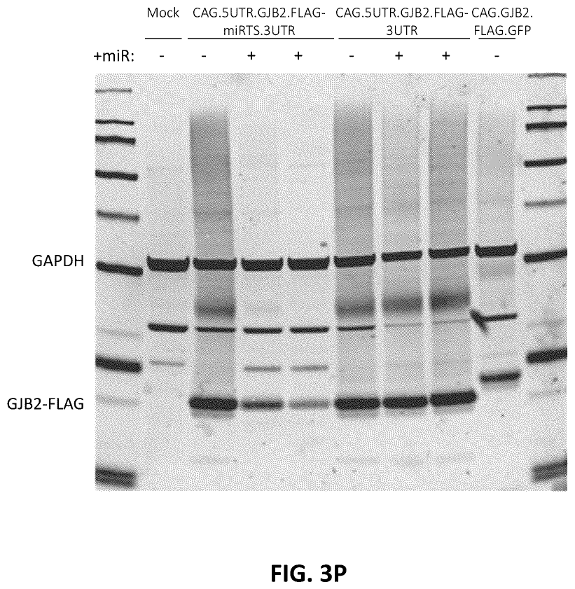

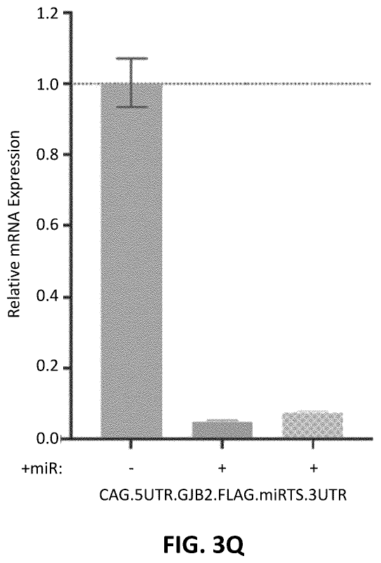

To regulate transgene mRNA expression only in supporting cells, a construct comprising microRNA target site was created. In cell types where the transgene (e.g., a supporting cell gene or a GJB2 gene) may not be well-tolerated (e.g., hair cells), a microRNA recognizes the miRTS and degrades the mRNA, preventing its expression. A schematic of the construct is shown in FIG. 3A. The construct comprises a promoter, 5′ UTR, transgene (GOI), miRTs, 3′ UTR, and polyA, flanked by inverted terminal repeats. To prevent expression of a transgene in hair cells and allow expression in other cochlear cell types, such as supporting cells, microRNAs expressed in hair cells but not in supporting cells can be used (FIG. 3B).

An in vitro experiment was conducted with a plasmid comprising eGFP and a miRTS (eGFP-miRTS) that was co-transfected into HEK293FT cells with a plasmid expressing a microRNA targeting the miRTS (pITR.CMV.mScarlet.miRNA) at range of miRNA to target DNA ratios. eGFP expression was measured by flow cytometry and normalized to eGFP-miRTS expression in the absence of the microRNA. Increasing ratios of pITR.CMV.mScarlet.miRNA to eGFP-miRTS led to a reduction in eGFP expression (FIG. 3C). When cells were transduced with an Anc80 particle comprising a construct expressing eGFP without a miRTS (eGFP) (AAVAnc80-CAG.eGFP) alongside transfection with pITR.CMV.mScarlet.miRNA, a reduction in expression of eGFP was not observed by flow cytometry. Transduction of cells with a contruct comprising eGFP and a miRTS (AAVAnc80-CAG.eGFP.miRTS) alongside transfection of the cells with pITR.CMV.mScarlet.miRNA resulted in decreased eGFP expression (FIG. 3D).

A similar effect on expression of a gene of interest (GOI (GJB2)) was observed when HEK293FT cells were transduced with an AAVAnc80 vector comprising a gene of intrest and miRTS site in its 3′UTR (AAVAnc80-CAG.GOI.miRTS) alongside transfection with pITR.CAG.mScarlet.miRNA, which expresses a microRNA targeting the miRTS. A reduction in the gene of interest mRNA was measured by real-time qPCR and western blotting when cells were transfected with either 100 ng or 200 ng of pITR.CAG.mScarlet.miRNA alongside transduction with AAVAnc80-CAG.GOI.miRTS compared to transduction of AAVAnc80-CAG.GOI.miRTS alone (FIGS. 3E and 3F).

In contrast, transfection of pITR.CAG.mScarlet-miRNA did not reduced the level of the gene of intrest when cells were transduced with a construct comprising the gene of interest without the 3′UTR miRTS (AAVAnc80-CAG.GOI) (FIG. 3G). The reduction in protein level observed by Western blot following AAVAnc80-CAG.GOI.miRTS transduction and pITR.CAG.mScarlet-miRNA was quantified in FIG. 3H.

In order to determine whether there were any substantial off-target effects on gene expression caused by transduction with AAVAnc80-CAG.GOI.miRTS, bulk RNA sequencing was performed. Cluster analysis failed to detect any clear sample clustering based on the transduction with or without the miRTS (FIG. 3I). Differential expression analysis was used to identify genes that were significantly upregulated or downregulated by the expression of AAVAnc80-CAG.GOI.miRTS compared to AAVAnc80-CAG.GOI. The only genes significantly altered in expression were associated with T-cell immune response (FIG. 3J), further indicating that there were not significant off-target effects on gene expression caused by the expression of the miRTS-containing construct.

Cochlear explant cultures were transduced with an AAVAnc80 virus expressing a FLAG-tagged gene of interest (GOI) with a miRTS targeted by miRNAs expressed in hair cells (AAVAnc80-CAG.GOI.FLAG.miRTS1-4) or without a miRTS (AAVAnc80-CAG.GOI.FLAG). FIG. 3K (left) shows an untreated cochlear explant with hair cells labeled with MYO7A in red. Following transduction with AAVAnc80-CAG.GOI.FLAG the gene of interest is expressed in hair cells (indicated by white arrowheads) and in both lateral and medial supporting cells (FIG. 3K, right). In contrast, following transduction of AAVAnc80-CAG.GOI.FLAG.miRTS1, AAVAnc80-CAG.GOI.FLAG.miRTS2, and AAVAnc80-CAG.GOI.FLAG.miRTS3, expression of the gene of interest was limited to supporting cells, as observed by FLAG labeling (green) (FIGS. 3L, 3M, and 3N). Of the constructs expressing the gene of interest with a miRTS in the 3′UTR, only AAVAnc80-CAG.GOI.FLAG.miRTS4 was unsuccessful in limiting expression to supporting cells. FLAG labeling of the gene of interest is colocalized with MYO7A (white arrowhead) in some hair cells (FIG. 3O).

Experiments were conducted to demonstrate mRNA expression regulation from rAAV constructs transfected into HEK293FT cells. rAAV constructs comprising hGJB2.FLAG (CAG.SUTR.hGJB2.FLAG.3UTR; SEQ ID NO: 82) and optional miRNA regulatory target sites (miRTS) located in the 3′UTR (CAG.SUTR.hGJB2.FLAG.miRTS.3UTR; FIG. 2F; SEQ ID NO: 87) were transfected into HEK293FT cells at 300 ng with (+) or without (−) an additional plasmid comprising miRNA coding regions (e.g., miR-182, and miR-183) transfected at 400 ng. At 72 h post transfection the cells were harvested for GJB2 protein and RNA analysis using western blot analysis (see FIG. 3P) and real-time qPCR (see FIG. 3Q). Reduction in GJB2 RNA and protein expression was detected in samples that were co-expressing the target plasmid and miR-182 and miR-183 compared to samples expressing the target plasmid alone. Similar hGJB2.FLAG comprising plasmids that did not include miR-182 and miR-183 target sites were used as control and presented similar hGJB2 protein levels with and without miR-182 and miR-183 co-expression (see FIG. 3P and FIG. 3Q).

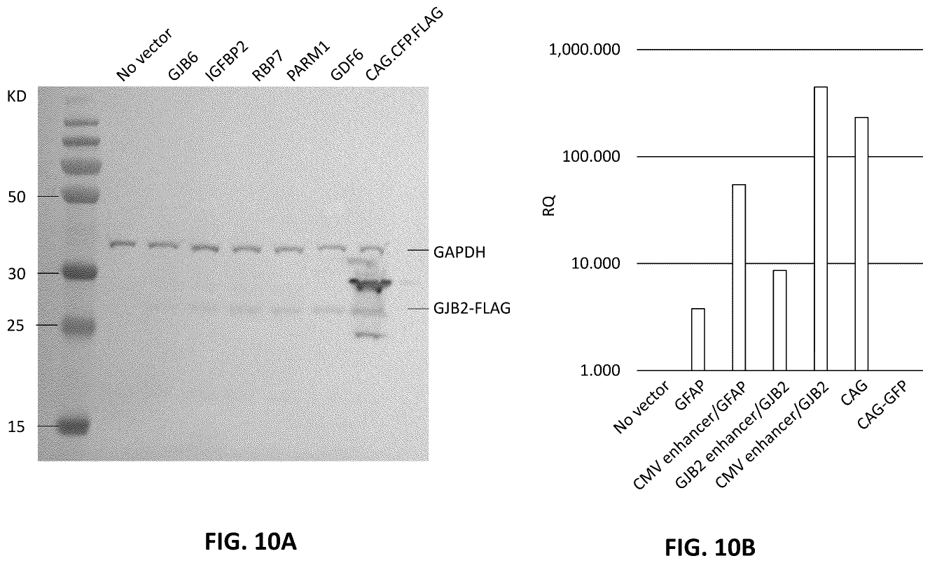

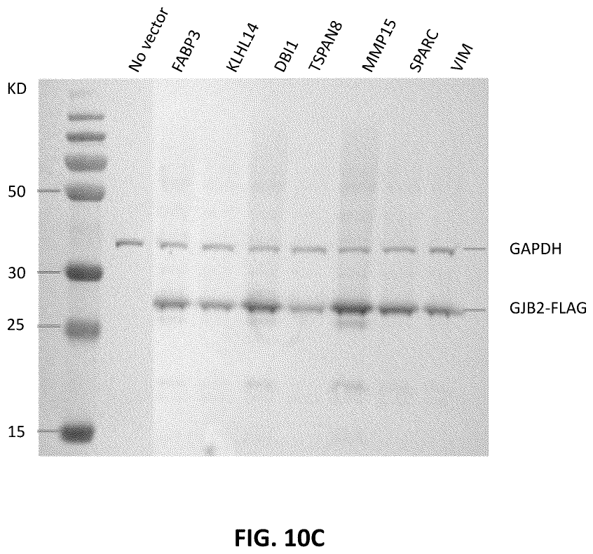

rAAV particles comprising rAAV constructs containing a GJB2 flag tagged polynucleotide operably linked to a supporting cell selective promoter (GFAP, GJB6, IGFBP2, RPB7, PARM1, or GDF6) in combination with a minimal GJB2 promoter were transduced into HEK293FT cells. Expression by a CAG promoter was used as a positive control. Protein and RNA analysis shows that each these constructs were able to express Connexin 26 (FIGS. 10A-10B).

Plasmids comprising a GJB2 flag tagged polynucleotide operably linked to a supporting cell selective promoter (FABP3, KLHL14, DBI2, TSPAN8, MMP15, SPARC, or VIM) in combination with a minimal GJB2 promoter were transfected into HEK293FT cells. Connexin 26 expression was observed from all constructs by western blot (FIG. 10C).

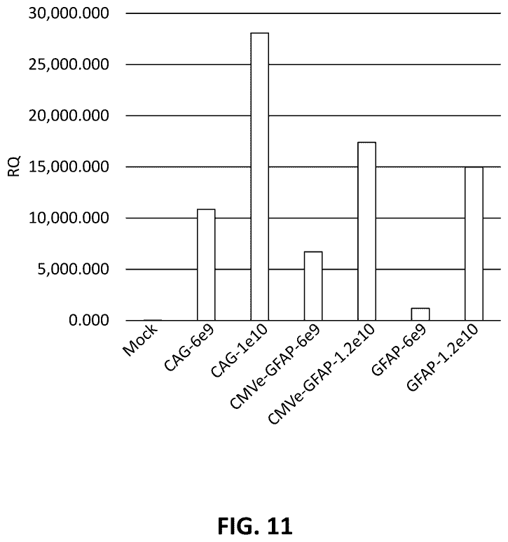

rAAV particles comprising rAAV contructs were encapsidated by Anc80 capisids and transduced into neonate cochlear explants at different doses. RNA analysis shows that GJB2 mRNA expression increased with dosing (FIG. 11).

Those of ordinary skill in the art will readily understand that there are alternative methods of conducting the experiments associated with the current example, for instance, alternative viral titers, MOIs, cell concentrations, time to cellular harvest, reagents utilized for cellular harvesting or mRNA or protein analysis, AAV serotypes, and/or standard modifications to a construct comprising an gene are practical and expected alterations of the current example.

### Example 6: Preliminary Hair Cell Tolerability Assessment of Transgenic GJB2 mRNA Expression and Connexin 26 Protein Production in Neonate Cochlear Explants

This example relates to the introduction, and expression analysis of rAAV constructs overexpressing a GJB2 gene in neonatal cochlear explants. Mock rAAV particles or rAAV particles comprising rAAV constructs (FIG. 2 panels (A)-(H)) encapsidated by Anc80 capsids are prepared and transduced into neonate cochlear explants at a known MOI (e.g., approximately 4.5×10⁹ or 1.3×10¹⁰ vg/per cochlea). Explants are grown to levels appropriate for harvest (e.g., for 72 hours post transduction), and are then prepared for immunofluorescence staining/imaging through fixation using 4% PFA or RNA extraction. RNA samples are prepared and GJB2 gene overexpression is confirmed using quantitative PCR with appropriate reagents in a manner described in a published method (e.g., appropriate according to the RNeasy Micro Kit and quantitative real-time PCR) using construct specific primers and relative to a control. Robust GJB2 mRNA production is observed in explants transduced with test rAAV when compared to mock transduction events. Tolerability and lack of hair cell toxicity is determined using immunofluorescence staining/imaging, antibodies targeting Myo7a (Proteus Biosciences) are utilized to depict inner ear hair cells, while DAPI staining is used to define nuclear positioning. No or low hair cell (Myo7) toxicity is observed after GJB2 overexpression.

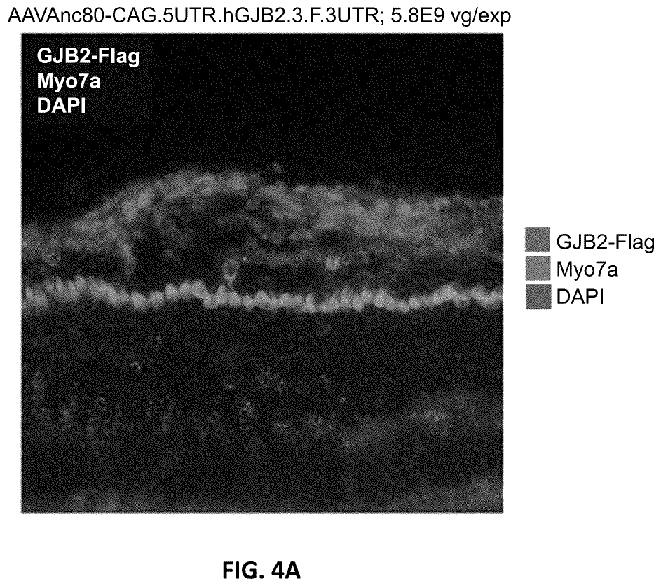

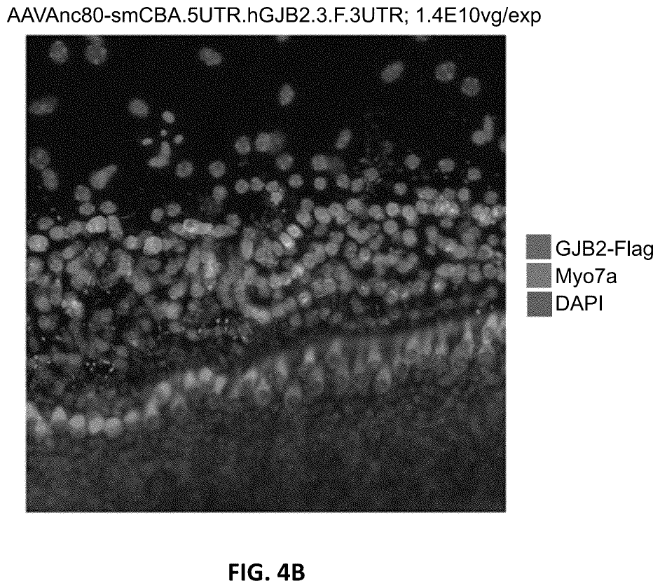

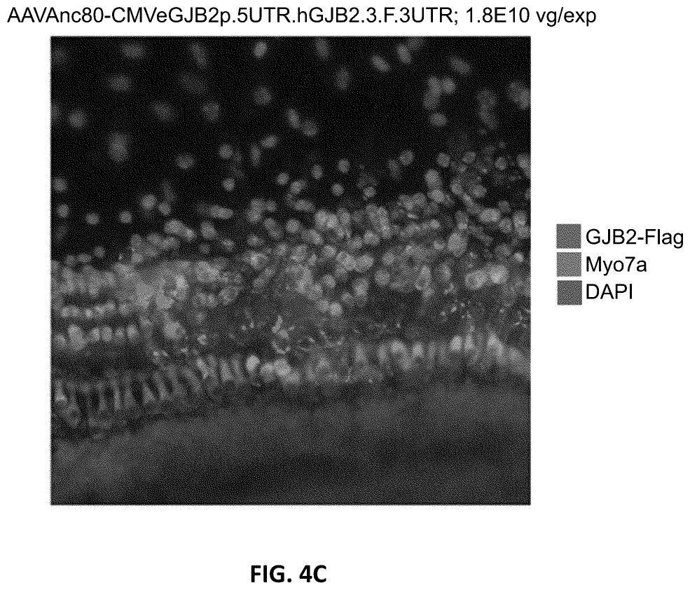

rAAV Anc80 particles comprising rAAV constructs driven by CAG, CMVe-GJB2p, or smCBA promoter/enhancer combinations were prepared and transduced into mouse neonate (P2) cochlear explants at a known MOI (approximately 5.8×10⁹, 1.4×10¹⁰, or 1.8×10¹⁰ vg/per cochlea respectively). Explants were grown to levels appropriate for harvest (e.g., for 72 hours post transduction), and were then prepared for immunofluorescence staining/imaging through fixation using 4% PFA. Explants were then DAPI stained (presented in blue) and immunostained using anti-FLAG antibodies (presented in green), and hair cell specific anti-Myo7a antibodies (presented in red), explants were subsequently imaged (exemplary data presented in FIG. 4). Robust FLAG signal was observed in the supporting cells of the explants transduced with rAAV particles comprising AAVAnc80-CAG.SUTR.hGJB2.3F.3UTR (as depicted in FIG. 2 panel (C), SEQ ID NO: 82) at 5.8E9 vg/explant (see FIG. 4 panel (A)). Robust FLAG signal was observed in the supporting cells in explants transduced with rAAV particles comprising AAVAnc80-smCBA.SUTR.hGJB2.3F.3UTR (as depicted in FIG. 2 panel (D), SEQ ID NO: 83) at 1.4E10 vg/explant (see FIG. 4 panel (B)). Robust FLAG signal was observed in the supporting cells of the explants transduced with rAAV particles comprising AAVAnc80-CMVeGFAPp.5UTR.hGJB2.3F.3UTR (as depicted in FIG. 2 panel (E), SEQ ID NO: 84) at 1.8E10 vg/explant (see FIG. 4 panel (C)). Variation in FLAG expression was detected across samples, likely the results of variability in vector titer.

### Example 7: Surgical Method in Aged Mice

The current example relates to the introduction of constructs described herein to the inner ear of aged mice. rAAV particles comprising an AAV capsid and a construct encoding a connexin 26 protein or characteristic functional portion thereof are prepared in formulation buffer (e.g., artificial perilymph or 1×PBS with pluronic acid F68) and then administered to the scala tympani in mice as described by Shu et al., Human Gene Therapy, 27(9):687-699, 2016, which is incorporated in its entirety herein by reference). Male and female mice older than P15 are anesthetized using an intraperitoneal injection of xylazine (e.g., approximately 5-10 mg/kg) and ketamine (e.g., approximately 90-120 mg/kg). Body temperature is maintained at 37° C. using an electric heating pad. An incision is made from the right post-auricular region and the tympanic bulla and posterior semicircular canal are exposed. The bulla is perforated with a surgical needle and the small hole is expanded to provide access to the cochlea. The bone of the cochlear lateral wall of the scala tympani is thinned with a dental drill so that the membranous lateral wall is left intact. A small hole is then drilled in the posterior semicircular canal (PSCC). Patency of the canalostomy is confirmed by visualization of a slow leak of perilymph. A Nanoliter Microinjection System in conjunction with glass micropipette is used to deliver a total of approximately 1 μL of construct containing buffer (e.g., rAAV constructs described herein at approximately 4.5×10⁹ to 5×10¹⁰ vg/per cochlea in artificial perilymph or 1×PBS with pluronic acid F68) to the scala tympani at a rate of approximately 2 nL/second. The glass micropipette is left in place for 5 minutes post-injection. Following cochleostomy and injection, the opening in the tympanic bulla and the PSCC are sealed with small pieces of fat, and the muscle and skin are sutured. The mice are allowed to awaken from anesthesia and their pain is controlled with 0.15 mg/kg buprenorphine hydrochloride for 3 days.

### Example 8: Transgenic Expression and Imaging of Connexin 26 Protein in Wild-Type Mice

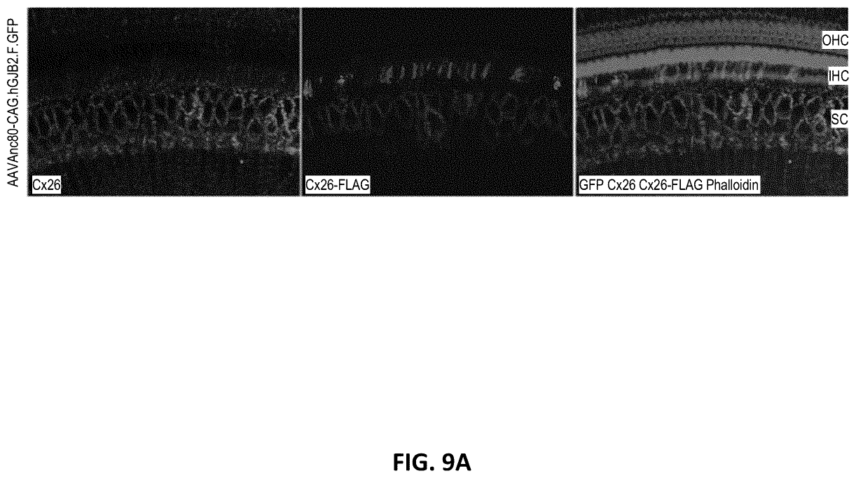

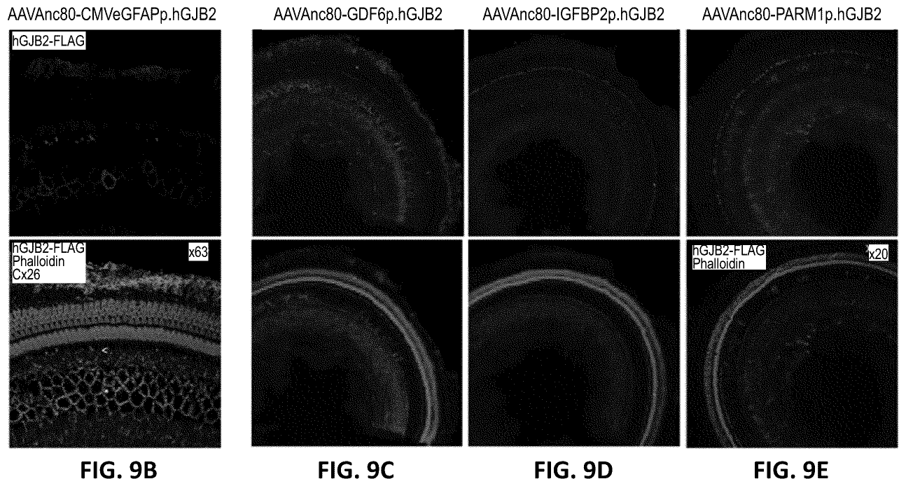

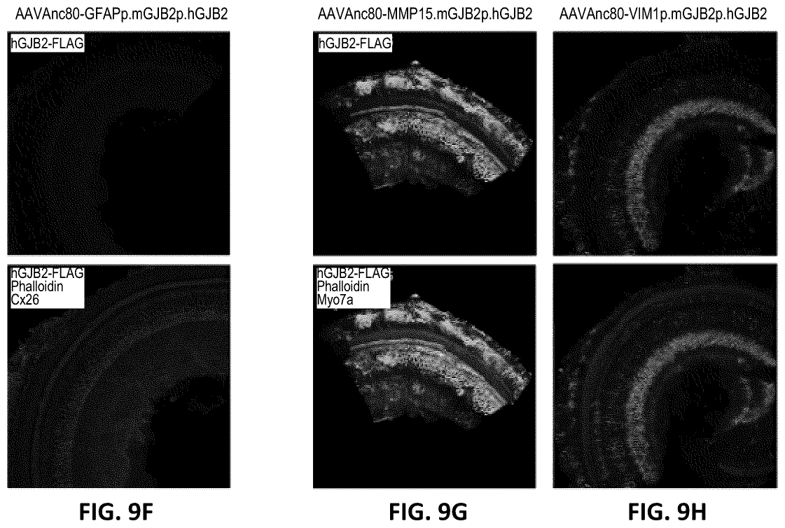

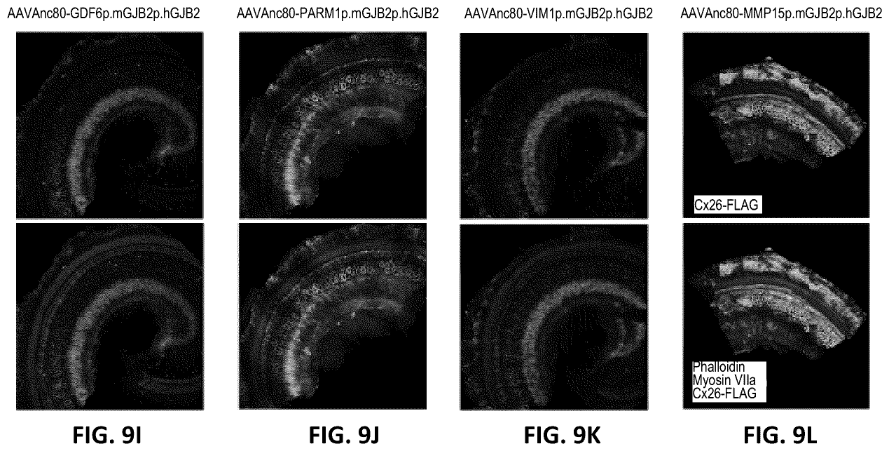

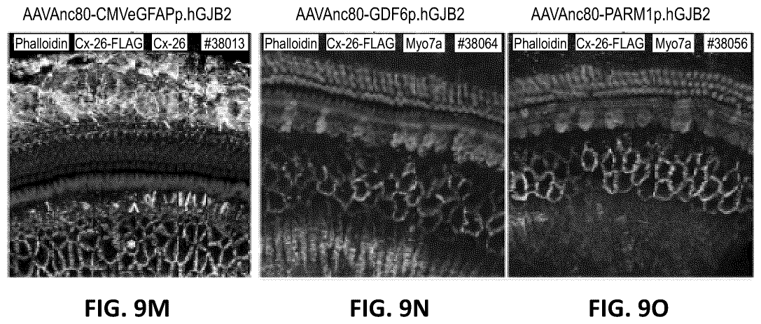

This example relates to the transgenic expression and analysis of transgenic connexin 26 protein in wild-type mice and GJB2 inducible conditional knockout mice. Wild-type mice were administered AAVAnc80 particles (1.2×10¹⁰ vg/cochlea) comprising CAG.hGJB2.FLAG.GFP (schematic provided in FIG. 2H) to the cochlea by the method described in Example 7. 10 days after administration clear and robust of exogenous Connexin 26 (FLAG; purple) was detected in the membrane of the supporting cells of the sensory epithelia (FIG. 9A, middle and right panels). Expression of exogenous Connexin 26 was also detected in the inner hair cells. Endogenous Connexin 26 (red) was detected in all supporting cells (FIG. 9A, left and right panels).

Juvenile wild-type mice were administered 1 μl of AAVAnc80 particles comprising AAVAnc80-CMVeGFAPp.5UTR.hGJB2.FLAG.3UTR (SEQ ID NO: 84), AAVAnc80-GDF6p.mGJB2p.5UTR.hGJB2.FLAG.3UTR (construct comprising SEQ ID NO: 61 and supporting cell selective promoter comprising SEQ ID NO: 90), AAVAnc80-IGFBP2p.mGJB2p.5UTR.hGJB2.FLAG.3UTR (construct comprising SEQ ID NO: 54 and supporting cell selective promoter comrpising SEQ ID NO: 57), AAVAnc80-PARM1p.mGJB2p.5UTR.hGJB2.FLAG.3UTR (construct comprising SEQ ID NO: 7 and supporting cell selective promoter comprising SEQ ID NO: 40), AAVAnc80-GFAPp.mGJB2p.hGJB2, AAVAnc80-MMP15p.mGJB2p.hGJB2, or AAVAnc80-VIMp.mGJB2p.hGJB2. Administration of a hGJB2 construct with a promoter incorporating the CMV-enhancer resulted in supporting cell expression that colocalized with endogenous connexin 26 expression (FIG. 9B; asterisk). However, inner hair cell expression was still detected (arrowhead). In contrast, administration of a hGJB2 construct with promoters derived from supporting cell genes GDF6 (FIG. 9C), IGFBP2 (FIG. 9D), and PARM1 (FIG. 9E) in combination with a minimal GJB2 promoter resulted in supporting cell expression without detection of inner hair cell expression.

Administration of AAVAnc80 particles comprising AAVAnc80-GFAPp.mGJB2p.hGJB2 did not result in supporting cell expression of GJB2 (FIG. 9F). Administration of AAVAnc80 particles comprising AAVAnc80-MMP15p.mGJB2p.hGJB2 or AAVAnc80-VIMp.mGJB2p.hGJB2 resulted in supporting cell expression of flag-tagged hGJB2. Expression of flag-tagged hGJB2 not detected in hair cells as noted by Myo7a staining. Likewise, administration of a hGJB2 construct with promoters derived from supporting cell genes GDF6 (FIG. 9I), PARM1 (FIG. 9J), VIM (FIG. 9K), and MMP15p (FIG. 9L) resulted in supporting cell expression without detection of inner hair cell expression.

Juvenile WT mice were administered with AAVAnc80 particles comprising AAVAnc80.CMVe.GFAP.mGJB2p.hGJB2.FLAG, AAVAnc80.CMVe.GDF6.mGJB2p.hGJB2.FLAG, or AAVAnc80.CMVe.PARM1.mGJB2p.hGJB2.FLAG through the round window membrane with posterior semicircular canal fenestration. 4 weeks post administration, the mice were euthanized, the inner ears were harvested in fixed in PFA, and the injected (left) ear was processed for immunofluorescent staining using phalloidin to label all cells and hair-cells stereocilia bundle, anti-FLAG to label the transgene and the hair cell marker Myo7a. Anti-Cx26 antibody was also used in some of the samples to colocalize the expression of the transgene with endogenous Cx26 expression. Multiple regions from the base, middle and apex of the cochlea were imaged and represented images are presented in FIGS. 9M-9O.

Expression of the Cx26-FLAG transgene (green) was detected in supporting cells, overlapping with endogenous Cx26 expression (asterisk), and in some cases, was also observed in inner hair cells (arrowhead) (FIG. 9M). FIGS. 9N and 9O demonstrate robust FLAG expression in supporting cells (green) with no apparent expression in IHCs or hair cell loss. Futher, differential expression patterns were observed between supporting cell subtypes.

1-7. (canceled)

8. An expression construct comprising a polynucleotide encoding a polypeptide operably linked to a promoter, wherein the promoter comprises a nucleic acid sequence having at least 85%, at least 90%, at least 95%, at least 96%, at least 97%, at least 98%, at least 99%, or 100% identity to any one of SEQ ID NOs: 16, 28, 40, 57, or 90-99.

9. The expression construct of claim 8, wherein the promoter is heterologous to the polynucleotide.

10. The expression construct of claim 8, wherein the promoter is capable of directing transcription of the polynucleotide in an inner ear support cell.

11. (canceled)

12. The expression construct of claim 8, wherein the inner ear support cell is selected from one or more of inner phalangeal cells/border cells (IPhC), inner pillar cells (IPC), outer pillar cells (OPC), Deiters' cells rows 1 and 2 (DC1/2), Deiters' cells row 3 (DC3), Hensen's cells (Hec), Claudius cells/outer sulcus cells (CC/OSC), interdental cells (Idc), inner sulcus cells (ISC), Kölliker's organ cells (KO), greater ridge epithelial ridge cells (GER) (including lateral greater epithelial ridge cells (LGER)), and OC90+ cells (OC90), fibroblasts, and other cells of the lateral wall.

13. The expression construct of claim 8, wherein the promoter comprises a nucleic acid sequence having at least 85%, at least 90%, at least 95%, at least 96%, at least 97%, at least 98%, at least 99%, or 100% identity to any one of SEQ ID NOs: 40, 90, 96, or 99.

14. The expression construct of claim 8 further comprising a miRNA regulatory target site (miRTS) for a microRNA expressed in an inner ear cell.

15. The expression construct of claim 14, wherein the microRNA is expressed in an inner ear hair cell.

16. The expression construct of claim 15, wherein the microRNA is one or more of miR-194, miR-140, miR-18a, miR-99a, miR-30b, miR-15a, miR182, or miR-183.

17. The expression construct of claim 8, further comprising a minimal GJB2 promoter which is operably linked to the nucleic acid sequence encoding the polypeptide.

18-86. (canceled)

87. The expression construct of claim 8, wherein the construct further comprises a 5′ UTR and a 3′ UTR.

88-90. (canceled)

91. The expression construct of claim 87, wherein the 3′ UTR and/or the 5′ UTR comprises a miRTS.

92. The expression construct of claim 8, further comprising a polyA tail.

93-105. (canceled)

106. The expression construct of claim 8, wherein the construct is selectively expressed in an inner ear supporting cell.

107-108. (canceled)

109. A viral vector comprising the expression construct of claim 8.

110-112. (canceled)

113. The viral vector of claim 109, wherein the viral vector is an AAV vector.

114. An AAV particle comprising the expression construct of claim 8.

115-116. (canceled)

117. A composition comprising the expression construct of claim 8.

118. The composition of claim 117, wherein the composition is a pharmaceutical composition further comprising a pharmaceutically acceptable carrier.

119. (canceled)

120. An ex vivo cell comprising the expression construct of claim 8.

121-128. (canceled)

129. A method of expressing the polypeptide in an inner ear supporting cell, comprising administering the expression construct of claim 8 to the inner ear supporting cell.

130. (canceled)

131. A method of increasing expression of the polypeptide in an inner ear supporting cell, comprising administering the expression construct of claim 8 to the inner ear supporting cell.

132. (canceled)

133. The method of claim 129, wherein the expression of the polypeptide is reduced, suppressed, or eliminated in non-inner ear supporting cells compared to endogenous expression of the polypeptide in non-inner ear supporting cells.

134.-141. (canceled)

142. A method of treating hearing loss in a subject suffering from or at risk of hearing loss, comprising administering the expression construct of claim 8 to the subject.

143.-156. (canceled)

157. A kit comprising the expression construct of claim 8.

158.-166. (canceled)

167. The viral vector of claim 109, comprising a 5′ and a 3′ inverted terminal repeat (ITR).


SEQUENCE LISTING (118 sequences)

SEQ ID NO: 1 - DNA, 22 bp, synthetic construct
  source 1..22: mol_type = other DNA; note = Exemplary mRNA destabilizing domain sequence
tccacatggagttgctgttaca

SEQ ID NO: 2 - DNA, 21 bp, synthetic construct
  source 1..21: mol_type = other DNA; note = Exemplary mRNA destabilizing domain sequence
ccgtggttctaccctgtggta

SEQ ID NO: 3 - DNA, 23 bp, synthetic construct
  source 1..23: mol_type = other DNA; note = Exemplary mRNA destabilizing domain sequence
ctatctgcactagatgcacctta

SEQ ID NO: 4 - DNA, 22 bp, synthetic construct
  source 1..22: mol_type = other DNA; note = Exemplary mRNA destabilizing domain sequence
cacaagatcggatctacgggtt

SEQ ID NO: 5 - DNA, 20 bp, synthetic construct
  source 1..20: mol_type = other DNA; note = Exemplary mRNA destabilizing domain sequence
ctgagtgtaggatgtttaca

SEQ ID NO: 6 - DNA, 22 bp, synthetic construct
  source 1..22: mol_type = other DNA; note = Exemplary mRNA destabilizing domain sequence
cacaaaccattatgtgctgcta

SEQ ID NO: 7 - DNA, 6915 bp, synthetic construct
  source 1..6915: mol_type = other DNA; note = Exemplary construct sequence
cctgcaggcagctgcgcgctcgctcgctcactgaggccgcccgggcgtcgggcgacctttggtcgcccggcctcagtgagcgagcgagcgcgcagagagggagtggccaactccatcactaggggttcctgcggccgcacgcgtggttgtacaggagatagtcagggaattagtaattttcaaagaggtgactttgaattcaaacttaaatatcatcttcagctgaaacaaagaaggggtgcagttatgaggaagtgaccaggtaaagcatggcaaacaaaggtaaagtttgttatgcgtatttaagtcagagccctctccattgataagagtttccagtaatttagtgccatccttttcttgctatagagttctcgtctctatctgagcacgcaaaaataacatgctttcttgctttcttgaagttgggcatggccattgacttgccttagcccatatttttctgtgaagtggtcttcaaaaacctatatttctgccatagagtcacttacttaacctgccctatttaaaggggctaatgcctgatagaatgtcgctgcataactccatctgtgtgtggtccctgcatccatgacaaccaaaacccagatgcagaaattgttcctaatcacatagattaccctagaaaccggaagggccttgaagtcaaaagcattcagagaacatgctgaacaaattgaatttgcagtttatctggccagggaggatggagaggggatgggcacttggtctgagtattttttgtttctcattccaacagaaattactagatttaccaaaaaatctacaagtggtagtgtgatagagtcaggcagaggaattgaccatagataaggtgctcaggactcctagagtcagcttctggtatgtgagaaagaagtgagaacagagcccatggcatatgaagaagatattacagaaaaaagaaagctgccttccacgcaaatcatttctttacaaaggcttgttaactcctgcagtgccaagaagctgaatgcagcggcagacatcctggttcgggccccaggaagctcagccgggtttaatgtggatgagggtttaatgatgtacacgcagaagtgttttgacaaatgaagaaggtcctcattcttggaacatgtgccggttctccgagggaactcctaaaaggctgtaagctcatgtaggaaaagctgagctagattcctaagggcagagatgtgctcacatttctttgcatccctagttcccagcacagtgcaaggcgctgcaaacatttgctgaacccagggtctcgtgtcttgactgtccagcagaggccgctctgggccggggctctcgggacctgagggctgagagaaggaaggccagggggtggcccagtcatcgccgcggggcccgggtgggaggggtttggcagcggcaggcgcggcggcggcggcggaggcggaggcggccccgggaagctctgaggacccagaggccgggcgcgctccgcccgcggcgccgccccctccgtaactttcccagtctccgagggaagaggcggggtgtggggtgcggttaaaaggcgccacggcgggagacaggtctcaccggtgcaaactggttgcggccccgcagcgcccgcgcgctcctctccccgactcggagcccctcggcggcgcccggcccaggacccgcctaggagcgcaggagccccagcgcagagaccccaacgccgagacccccgccccggccccgccgcgcttcctcccgacgcagtttaggacccttgttcgcgaagaggtggtgtgcggctgagacccgcgtcctcaggacggttccatcagtgcctcgatcctgccccactggaggaggaaggcagcccgaacagcgctcacctaactaacagctgctgagagctgggttccgtggccatgcacctgggactgccttgagaagcgtgagcaaaccgcccagagtagaagcgctagccaccatggattggggcacgctgcagacgatcctggggggtgtgaacaaacactccaccagcattggaaagatctggctcaccgtcctcttcatttttcgcattatgatcctcgttgtggctgcaaaggaggtgtggggagatgagcaggccgactttgtctgcaacaccctgcagccaggctgcaagaacgtgtgctacgatcactacttccccatctcccacatccggctatgggccctgcagctgatcttcgtgtccacgccagcgctcctagtggccatgcacgtggcctaccggagacatgagaagaagaggaagttcatcaagggggagataaagagtgaatttaaggacatcgaggagatcaaaacccagaaggtccgcatcgaaggctccctgtggtggacctacacaagcagcatcttcttccgggtcatcttcgaagccgccttcatgtacgtcttctatgtcatgtacgacggcttctccatgcagcggctggtgaagtgcaacgcctggccttgtcccaacactgtggactgctttgtgtcccggcccacggagaagactgtcttcacagtgttcatgattgcagtgtctggaatttgcatcctgctgaatgtcactgaattgtgttatttgctaattagatattgttctgggaagtcaaaaaagccagttggatcccgggctgactacaaagaccatgacggtgattataaagatcatgacatcgactacaaggatgacgatgacaagtaagaaatagacagcatgagagggatgaggcaacccgtgctcagctgtcaaggctcagtcgctagcatttcccaacacaaagattctgaccttaaatgcaaccatttgaaacccctgtaggcctcaggtgaaactccagatgccacaatggagctctgctcccctaaagcctcaaaacaaaggcctaattctatgcctgtcttaattttctttcacttaagttagttccactgagaccccaggctgttaggggttattggtgtaaggtactttcatattttaaacagaggatatcggcatttgtttctttctctgaggacaagagaaaaaagccaggttccacagaggacacagagaaggtttgggtgtcctcctggggttctttttgccaactttccccacgttaaaggtgaacattggttctttcatttgctttggaagttttaatctctaacagtggacaaagttaccagtgccttaaactctgttacactttttggaagtgaaaactttgtagtatgataggttattttgatgtaaagatgttctggataccattatatgttccccctgtttcagaggctcagattgtaatatgtaaatggtatgtcattcgctactatgatttaatttgaaatatggtcttttggttatgaatactttgcagcacagctgagaggctgtctgttgtattcattgtggtcatagcacctaacaacattgtagcctcaatcgagtgagacagactagaagttcctagtgatggcttatgatagcaaatggcctcatgtcaaatatttagatgtaattttgtgtaagaaatacagactggatgtaccaccaactactacctgtaatgacaggcctgtccaacacatctcccttttccatgactgtggtagccagcatcggaaagaacgctgatttaaagaggtcgcttgggaattttattgacacagtaccatttaatggggaggacaaaatggggcaggggagggagaagtttctgtcgttaaaaacagatttggaaagactggactctaaagtctgttgattaaagatgagctttgtctacttcaaaagtttgtttgcttaccccttcagcctccaattttttaagtgaaaatatagctaataacatgtgaaaagaatagaagctaaggtttagataaatattgagcagatctataggaagattgaacctgaatattgccattatgcttgacatggtttccaaaaaatggtactccacatatttcagtgagggtaagtattttcctgttgtcaagaatagcattgtaaaagcattttgtaataataaagaatagctttaatgatatgcttgtaactaaaataattttgtaatgtatcaaatacatttaaaacattaaaatataatctctataataagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggaagcttgaattcagctgacgtgcctcggaccgctaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaaccctatctcgggctattcttttgatttataagggattttgccgatttcggcctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgaacaataaaactgtctgcttacataaacagtaatacaaggggtgttatgagccatattcaacgggaaacgtcgaggccgcgattaaattccaacatggatgctgatttatatgggtataaatgggctcgcgataatgtcgggcaatcaggtgcgacaatctatcgcttgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccggaaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtctcgctcaggcgcaatcacgaatgaataacggtttggttgatgcgagtgattttgatgacgagcgtaatggctggcctgttgaacaagtctggaaagaaatgcataaacttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgt

SEQ ID NO: 8 - DNA, 145 bp, synthetic construct
  source 1..145: mol_type = other DNA; note = Exemplary 5' AAV ITR
ttggccactccctctctgcgcgctcgctcgctcactgaggccgcccgggcaaagcccgggcgtcgggcgacctttggtcgcccggcctcagtgagcgagcgagcgcgcagagagggagtggccaactccatcactaggggttcct

SEQ ID NO: 9 - DNA, 145 bp, synthetic construct
  source 1..145: mol_type = other DNA; note = Exemplary 3' AAV ITR
aggaacccctagtgatggagttggccactccctctctgcgcgctcgctcgctcactgaggccgggcgaccaaaggtcgcccgacgcccgggctttgcccgggcggcctcagtgagcgagcgagcgcgcagagagggagtggccaa

SEQ ID NO: 10 - DNA, 277 bp, synthetic construct
  source 1..277: mol_type = other DNA; note = Exemplary CBA promoter
gtcgaggtgagccccacgttctgcttcactctccccatctcccccccctccccacccccaattttgtatttatttattttttaattattttgtgcagcgatgggggcggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcg

SEQ ID NO: 11 - DNA, 279 bp, synthetic construct
  source 1..279: mol_type = other DNA; note = Exemplary CBA promoter
gtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcg

SEQ ID NO: 12 - DNA, 658 bp, synthetic construct
  source 1..658: mol_type = other DNA; note = Exemplary CMV/CBA enhancer/promoter
gacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatgggtcgaggtgagccccacgttctgcttcactctccccatctcccccccctccccacccccaattttgtatttatttattttttaattattttgtgcagcgatgggggcggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcg

SEQ ID NO: 13 - DNA, 660 bp, synthetic construct
  source 1..660: mol_type = other DNA; note = Exemplary CMV/CBA enhancer/promoter
gacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatgggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcg

SEQ ID NO: 14 - DNA, 1671 bp, synthetic construct
  source 1..1671: mol_type = other DNA; note = Exemplary CAG enhancer/promoter
gacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatgggtcgaggtgagccccacgttctgcttcactctccccatctcccccccctccccacccccaattttgtatttatttattttttaattattttgtgcagcgatgggggcggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgttgccttcgccccgtgccccgctccgcgccgcctcgcgccgcccgccccggctctgactgaccgcgttactcccacaggtgagcgggcgggacggcccttctcctccgggctgtaattagcgcttggtttaatgacggctcgtttcttttctgtggctgcgtgaaagccttaaagggctccgggagggccctttgtgcgggggggagcggctcggggggtgcgtgcgtgtgtgtgtgcgtggggagcgccgcgtgcggcccgcgctgcccggcggctgtgagcgctgcgggcgcggcgcggggctttgtgcgctccgcgtgtgcgcgaggggagcgcggccgggggcggtgccccgcggtgcgggggggctgcgaggggaacaaaggctgcgtgcggggtgtgtgcgtgggggggtgagcagggggtgtgggcgcggcggtcgggctgtaacccccccctgcacccccctccccgagttgctgagcacggcccggcttcgggtgcggggctccgtgcggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacag

SEQ ID NO: 15 - DNA, 1673 bp, synthetic construct
  source 1..1673: mol_type = other DNA; note = Exemplary CAG enhancer/promoter
gacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatgggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgttgccttcgccccgtgccccgctccgcgccgcctcgcgccgcccgccccggctctgactgaccgcgttactcccacaggtgagcgggcgggacggcccttctcctccgggctgtaattagcgcttggtttaatgacggctcgtttcttttctgtggctgcgtgaaagccttaaagggctccgggagggccctttgtgcgggggggagcggctcggggggtgcgtgcgtgtgtgtgtgcgtggggagcgccgcgtgcggcccgcgctgcccggcggctgtgagcgctgcgggcgcggcgcggggctttgtgcgctccgcgtgtgcgcgaggggagcgcggccgggggcggtgccccgcggtgcgggggggctgcgaggggaacaaaggctgcgtgcggggtgtgtgcgtgggggggtgagcagggggtgtgggcgcggcggtcgggctgtaacccccccctgcacccccctccccgagttgctgagcacggcccggcttcgggtgcggggctccgtgcggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacag

SEQ ID NO: 16 - DNA, 735 bp, synthetic construct
  source 1..735: mol_type = other DNA; note = GJB6 promoter
aaatagcttccaacgtttccaccccaccagcccttgcaccactccctgtactggccctgagctttctagtcttgactgaaaagcggggaggcaatgtggtctctcctggtgcactgtcccgaggaaggcctgctccgcttccccggaggagtcttcaaaggatggaggtaattaataaaaacaacccctgtacctcctctaagtggtcattaattaataaagaacctccaggctcctataggagaggtctgtgcaccccgcgggctatgagaaggctggatcacccagaaagactgaggatgtgtcctggcaaaaacacagcctgcccctcacactgctccccacgggtgcactagggaggaagagttccctcgagggcctgagcaggcgccccacacctgcacccgtgcagagggggctgggcccgccctctgcgctcccgagggagagccctaccccctgcatccccggtaccccgttccctccaagggccggaaagagggccccgcgcactgtgcacttcttaggggtcccccaccctgcgcccccgccacgggaaaaaggtccccgctctgcgcatccggccccggagggacagccccggtcctgcactccttgctcctcagggggacggtccgcgcccagcggctagtgcgccccgggtaggtgggggcggggggctcgtcgagtgacagcgctcgcctcccgcagcccgcccgagccgcgtcagggcag

SEQ ID NO: 17 - DNA, 6696 bp, synthetic construct
  source 1..6696: mol_type = other DNA; note = Exemplary Construct Sequence
cctgcaggcagctgcgcgctcgctcgctcactgaggccgcccgggcgtcgggcgacctttggtcgcccggcctcagtgagcgagcgagcgcgcagagagggagtggccaactccatcactaggggttcctgcggccgcacgcgtggtcccatggctctgttaaaatcaaagaaacatcttttccaacagccctttcaaactcctcatcgcatctcactggctgattcagtcatttaaacctgcttctccctaaagctgatcactggctaagctaatagggtttccgggattggtttagcctgatactaatccaggtctaccttcaggagccagaccaaactgcctattggcattgcattcttgcagtagggaggggaggtatggatggtgtggagtccaccacaaggtccatgccagtctttgctgaaccagcatcagactccatcaagcaacagatgagaggttccatgataaagtggccctcagcaatccccatccattgctgtctaggaagaacagtgcttgtacacaggtttaggacctcagtcttggctgtaatcttctggtttactttgccagcaccaaacagaaggaaagaaagggctcaaatttgaccaaataaattatgcttctccttccagagataaccttgagtcctgtctaggaagatattagaattgtaaagaaaaaaaaaattactccttatcctatggcaagtggagtctatgtctacttcagctgaaattaaatcctgtccataatagatgacccttgctcaagctggccagaagccataccaaccagcacgaaggttaaaactattattagttttttctgtgattttcattttcaggccaagttttagaacaataagattttaagaataggaagtaagtaagatttctgcatatcctgttctcttagtcagctgaatttttttttttttttttttagtcctaactcagcctcccaaagtgctgggattacaggcgtgagccaccgcaccaagcctggaatctatgtcttacagttatgagaatcaacagctagctcattatgggcaaggtgatgtcactctggcttctcaatgaaaatggcatttctcccttggaaaaggtcatagccagtcagtcagtcagtcacgggagcgcagcggcttctaggggtgagtgggacccacgcggccccacctgctcctcccgcgcgcggccccacccccctgccccgccccgcctggtttatagaagctctgaggacccagaggccgggcgcgctccgcccgcggcgccgccccctccgtaactttcccagtctccgagggaagaggcggggtgtggggtgcggttaaaaggcgccacggcgggagacaggtctcaccggtcgtgtgttgttgcggccccgcagcgcccgcgcgctcctctccccgactcggagcccctcggcggcgcccggcccaggacccgcctaggagcgcaggagccccagcgcagagaccccaacgccgagacccccgccccggccccgccgcgcttcctcccgacgcagtttaggacccttgttcgcgaagaggtggtgtgcggctgagacccgcgtcctcaggacggttccatcagtgcctcgatcctgccccactggaggaggaaggcagcccgaacagcgctcacctaactaacagctgctgagagctgggttccgtggccatgcacctgggactgccttgagaagcgtgagcaaaccgcccagagtagaagcgctagccaccatggattggggcacgctgcagacgatcctggggggtgtgaacaaacactccaccagcattggaaagatctggctcaccgtcctcttcatttttcgcattatgatcctcgttgtggctgcaaaggaggtgtggggagatgagcaggccgactttgtctgcaacaccctgcagccaggctgcaagaacgtgtgctacgatcactacttccccatctcccacatccggctatgggccctgcagctgatcttcgtgtccacgccagcgctcctagtggccatgcacgtggcctaccggagacatgagaagaagaggaagttcatcaagggggagataaagagtgaatttaaggacatcgaggagatcaaaacccagaaggtccgcatcgaaggctccctgtggtggacctacacaagcagcatcttcttccgggtcatcttcgaagccgccttcatgtacgtcttctatgtcatgtacgacggcttctccatgcagcggctggtgaagtgcaacgcctggccttgtcccaacactgtggactgctttgtgtcccggcccacggagaagactgtcttcacagtgttcatgattgcagtgtctggaatttgcatcctgctgaatgtcactgaattgtgttatttgctaattagatattgttctgggaagtcaaaaaagccagttggatcccgggctgactacaaagaccatgacggtgattataaagatcatgacatcgactacaaggatgacgatgacaagtaagaaatagacagcatgagagggatgaggcaacccgtgctcagctgtcaaggctcagtcgctagcatttcccaacacaaagattctgaccttaaatgcaaccatttgaaacccctgtaggcctcaggtgaaactccagatgccacaatggagctctgctcccctaaagcctcaaaacaaaggcctaattctatgcctgtcttaattttctttcacttaagttagttccactgagaccccaggctgttaggggttattggtgtaaggtactttcatattttaaacagaggatatcggcatttgtttctttctctgaggacaagagaaaaaagccaggttccacagaggacacagagaaggtttgggtgtcctcctggggttctttttgccaactttccccacgttaaaggtgaacattggttctttcatttgctttggaagttttaatctctaacagtggacaaagttaccagtgccttaaactctgttacactttttggaagtgaaaactttgtagtatgataggttattttgatgtaaagatgttctggataccattatatgttccccctgtttcagaggctcagattgtaatatgtaaatggtatgtcattcgctactatgatttaatttgaaatatggtcttttggttatgaatactttgcagcacagctgagaggctgtctgttgtattcattgtggtcatagcacctaacaacattgtagcctcaatcgagtgagacagactagaagttcctagtgatggcttatgatagcaaatggcctcatgtcaaatatttagatgtaattttgtgtaagaaatacagactggatgtaccaccaactactacctgtaatgacaggcctgtccaacacatctcccttttccatgactgtggtagccagcatcggaaagaacgctgatttaaagaggtcgcttgggaattttattgacacagtaccatttaatggggaggacaaaatggggcaggggagggagaagtttctgtcgttaaaaacagatttggaaagactggactctaaagtctgttgattaaagatgagctttgtctacttcaaaagtttgtttgcttaccccttcagcctccaattttttaagtgaaaatatagctaataacatgtgaaaagaatagaagctaaggtttagataaatattgagcagatctataggaagattgaacctgaatattgccattatgcttgacatggtttccaaaaaatggtactccacatatttcagtgagggtaagtattttcctgttgtcaagaatagcattgtaaaagcattttgtaataataaagaatagctttaatgatatgcttgtaactaaaataattttgtaatgtatcaaatacatttaaaacattaaaatataatctctataataagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggaagcttgaattcagctgacgtgcctcggaccgctaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaaccctatctcgggctattcttttgatttataagggattttgccgatttcggcctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgaacaataaaactgtctgcttacataaacagtaatacaaggggtgttatgagccatattcaacgggaaacgtcgaggccgcgattaaattccaacatggatgctgatttatatgggtataaatgggctcgcgataatgtcgggcaatcaggtgcgacaatctatcgcttgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccggaaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtctcgctcaggcgcaatcacgaatgaataacggtttggttgatgcgagtgattttgatgacgagcgtaatggctggcctgttgaacaagtctggaaagaaatgcataaacttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgt

SEQ ID NO: 18 - DNA, 381 bp, synthetic construct
  source 1..381: mol_type = other DNA; note = Exemplary CMV enhancer
gacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatgg

SEQ ID NO: 19 - DNA, 1013 bp, synthetic construct
  source 1..1013: mol_type = other DNA; note = Exemplary SV-40 synthetic intron
ggagtcgctgcgttgccttcgccccgtgccccgctccgcgccgcctcgcgccgcccgccccggctctgactgaccgcgttactcccacaggtgagcgggcgggacggcccttctcctccgggctgtaattagcgcttggtttaatgacggctcgtttcttttctgtggctgcgtgaaagccttaaagggctccgggagggccctttgtgcgggggggagcggctcggggggtgcgtgcgtgtgtgtgtgcgtggggagcgccgcgtgcggcccgcgctgcccggcggctgtgagcgctgcgggcgcggcgcggggctttgtgcgctccgcgtgtgcgcgaggggagcgcggccgggggcggtgccccgcggtgcgggggggctgcgaggggaacaaaggctgcgtgcggggtgtgtgcgtgggggggtgagcagggggtgtgggcgcggcggtcgggctgtaacccccccctgcacccccctccccgagttgctgagcacggcccggcttcgggtgcggggctccgtgcggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacag

SEQ ID NO: 20 - DNA, 178 bp, synthetic construct
  source 1..178: mol_type = other DNA; note = Exemplary 5' UTR Sequence
gttgcggccccgcagcgcccgcgcgctcctctccccgactcggagcccctcggcggcgcccggcccaggacccgcctaggagcgcaggagccccagcgcagagaccccaacgccgagacccccgccccggccccgccgcgcttcctcccgacgcagagcaaaccgcccagagtagaag

SEQ ID NO: 21 - DNA, 206 bp, synthetic construct
  source 1..206: mol_type = other DNA; note = Exemplary 5' UTR Sequence
tttaggacccttgttcgcgaagaggtggtgtgcggctgagacccgcgtcctcaggacggttccatcagtgcctcgatcctgccccactggaggaggaaggcagcccgaacagcgctcacctaactaacagctgctgagagctgggttccgtggccatgcacctgggactgccttgagaagcgtgagcaaaccgcccagagtagaag

SEQ ID NO: 22 - DNA, 1432 bp, synthetic construct
  source 1..1432: mol_type = other DNA; note = Exemplary 3' UTR Sequence
cgcattgcccagttgttagattaagaaatagacagcatgagagggatgaggcaacccgtgctcagctgtcaaggctcagtcgctagcatttcccaacacaaagattctgaccttaaatgcaaccatttgaaacccctgtaggcctcaggtgaaactccagatgccacaatggagcctctgctcccctaaagcctcaaaacaaaggcctaattctatgcctgtcttaattttctttcacttaagttagttccactgagaccccaggctgttaggggttattggtgtaaggtactttcatattttaaacagaggatatcggcatttgtttctttctctgaggacaagagaaaaaagccaggttccacagaggacacagagaaggtttgggtgtcctcctggggttctttttgccaactttccccacgttaaaggtgaacattggttctttcatttgctttggaagttttaatctctaacagtggacaaagttaccagtgccttaaactctgttacactttttggaagtgaaaactttgtagtatgataggttattttgatgtaaagatgttctggataccattatatgttccccctgtttcagaggctcagattgtaatatgtaaatggtatgtcattcgctactatgatttaatttgaaatatggtcttttggttatgaatactttgcagcacagctgagaggctgtctgttgtattcattgtggtcatagcacctaacaacattgtagcctcaatcgagtgagacagactagaagttcctagtgatggcttatgatagcaaatggcctcatgtcaaatatttagatgtaattttgtgtaagaaatacagactggatgtaccaccaactactacctgtaatgacaggcctgtccaacacatctcccttttccatgactgtggtagccagcatcggaaagaacgctgatttaaagaggtcgcttgggaattttattgacacagtaccatttaatggggaggacaaaatggggcaggggagggagaagtttctgtcgttaaaaacagatttggaaagactggactctaaagtctgttgattaaagatgagctttgtctacttcaaaagtttgtttgcttaccccttcagcctccaattttttaagtgaaaatatagctaataacatgtgaaaagaatagaagctaaggtttagataaatattgagcagatctataggaagattgaacctgaatattgccattatgcttgacatggtttccaaaaaatggtactccacatatttcagtgagggtaagtattttcctgttgtcaagaatagcattgtaaaagcattttgtaataataaagaatagctttaatgatatgcttgtaactaaaataattttgtaatgtatcaaatacatttaaaacattaaaatataatctctataataa

SEQ ID NO: 23 - DNA, 82 bp, synthetic construct
  source 1..82: mol_type = other DNA; note = Exemplary splice donor intron
gtaagtatcaaggttacaagacaggtttaaggagaccaatagaaactgggcttgtcgagacagagaagactcttgcgtttct

SEQ ID NO: 24 - DNA, 51 bp, synthetic construct
  source 1..51: mol_type = other DNA; note = Exemplary splice acceptor intron
gataggcacctattggtcttactgacatccactttgcctttctctccacag

SEQ ID NO: 25 - DNA, 225 bp, synthetic construct
  source 1..225: mol_type = other DNA; note = Exemplary bGH poly(A) signal sequence
ctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatgg

SEQ ID NO: 26 - DNA, 122 bp, synthetic construct
  source 1..122: mol_type = other DNA; note = Exemplary SV40 poly(A) signal sequence
aacttgtttattgcagcttataatggttacaaataaagcaatagcatcacaaatttcacaaataaagcatttttttcactgcattctagttgtggtttgtccaaactcatcaatgtatctta

SEQ ID NO: 27 - skipped

SEQ ID NO: 28 - DNA, 1097 bp, synthetic construct
  source 1..1097: mol_type = other DNA; note = RBP7 promoter
cccatggctctgttaaaatcaaagaaacatcttttccaacagccctttcaaactcctcatcgcatctcactggctgattcagtcatttaaacctgcttctccctaaagctgatcactggctaagctaatagggtttccgggattggtttagcctgatactaatccaggtctaccttcaggagccagaccaaactgcctattggcattgcattcttgcagtagggaggggaggtatggatggtgtggagtccaccacaaggtccatgccagtctttgctgaaccagcatcagactccatcaagcaacagatgagaggttccatgataaagtggccctcagcaatccccatccattgctgtctaggaagaacagtgcttgtacacaggtttaggacctcagtcttggctgtaatcttctggtttactttgccagcaccaaacagaaggaaagaaagggctcaaatttgaccaaataaattatgcttctccttccagagataaccttgagtcctgtctaggaagatattagaattgtaaagaaaaaaaaaattactccttatcctatggcaagtggagtctatgtctacttcagctgaaattaaatcctgtccataatagatgacccttgctcaagctggccagaagccataccaaccagcacgaaggttaaaactattattagttttttctgtgattttcattttcaggccaagttttagaacaataagattttaagaataggaagtaagtaagatttctgcatatcctgttctcttagtcagctgaatttttttttttttttttttagtcctaactcagcctcccaaagtgctgggattacaggcgtgagccaccgcaccaagcctggaatctatgtcttacagttatgagaatcaacagctagctcattatgggcaaggtgatgtcactctggcttctcaatgaaaatggcatttctcccttggaaaaggtcatagccagtcagtcagtcagtcacgggagcgcagcggcttctaggggtgagtgggacccacgcggccccacctgctcctcccgcgcgcggccccacccccctgccccgccccgcctggtttatag

SEQ ID NO: 29 - DNA, 22 bp, synthetic construct
  source 1..22: mol_type = other DNA; note = Exemplary cloning site A
ttgtcgacgcggccgcacgcgt

SEQ ID NO: 30 - DNA, 39 bp, synthetic construct
  source 1..39: mol_type = other DNA; note = Exemplary cloning site B
ctcctgggcaacgtgctggttattgtgaccggtgccacc

SEQ ID NO: 31 - DNA, 24 bp, synthetic construct
  source 1..24: mol_type = other DNA; note = Exemplary cloning site C
taagagctcgctgatcagcctcga

SEQ ID NO: 32 - DNA, 38 bp, synthetic construct
  source 1..38: mol_type = other DNA; note = Exemplary cloning site D
aagcttgaattcagctgacgtgcctcggaccgcctagg

SEQ ID NO: 33 - skipped

SEQ ID NO: 34 - DNA, 15 bp, synthetic construct
  source 1..15: mol_type = other DNA; note = Exemplary cloning site F
gctgatcagcctcga

SEQ ID NO: 35 - DNA, 64 bp, synthetic construct
  source 1..64: mol_type = other DNA; note = Exemplary cloning site G
ggcattccggtactgttggtaaagccaccagcaaaccgcccagagtagaagaccggtggccacc

SEQ ID NO: 36 - DNA, 12 bp, synthetic construct
  source 1..12: mol_type = other DNA; note = Exemplary cloning site H
aagcttgaattc

SEQ ID NO: 37 - DNA, 26 bp, synthetic construct
  source 1..26: mol_type = other DNA; note = Exemplary cloning site I
agctgacgtgcctcggaccgcctagg

SEQ ID NO: 38 - DNA, 6334 bp, synthetic construct
  source 1..6334: mol_type = other DNA; note = Exemplary construct sequence
cctgcaggcagctgcgcgctcgctcgctcactgaggccgcccgggcgtcgggcgacctttggtcgcccggcctcagtgagcgagcgagcgcgcagagagggagtggccaactccatcactaggggttcctgcggccgcacgcgtggtaaatagcttccaacgtttccaccccaccagcccttgcaccactccctgtactggccctgagctttctagtcttgactgaaaagcggggaggcaatgtggtctctcctggtgcactgtcccgaggaaggcctgctccgcttccccggaggagtcttcaaaggatggaggtaattaataaaaacaacccctgtacctcctctaagtggtcattaattaataaagaacctccaggctcctataggagaggtctgtgcaccccgcgggctatgagaaggctggatcacccagaaagactgaggatgtgtcctggcaaaaacacagcctgcccctcacactgctccccacgggtgcactagggaggaagagttccctcgagggcctgagcaggcgccccacacctgcacccgtgcagagggggctgggcccgccctctgcgctcccgagggagagccctaccccctgcatccccggtaccccgttccctccaagggccggaaagagggccccgcgcactgtgcacttcttaggggtcccccaccctgcgcccccgccacgggaaaaaggtccccgctctgcgcatccggccccggagggacagccccggtcctgcactccttgctcctcagggggacggtccgcgcccagcggctagtgcgccccgggtaggtgggggcggggggctcgtcgagtgacagcgctcgcctcccgcagcccgcccgagccgcgtcagggcagaagctctgaggacccagaggccgggcgcgctccgcccgcggcgccgccccctccgtaactttcccagtctccgagggaagaggcggggtgtggggtgcggttaaaaggcgccacggcgggagacaggtctcaccggttcgtgggtgttgcggccccgcagcgcccgcgcgctcctctccccgactcggagcccctcggcggcgcccggcccaggacccgcctaggagcgcaggagccccagcgcagagaccccaacgccgagacccccgccccggccccgccgcgcttcctcccgacgcagtttaggacccttgttcgcgaagaggtggtgtgcggctgagacccgcgtcctcaggacggttccatcagtgcctcgatcctgccccactggaggaggaaggcagcccgaacagcgctcacctaactaacagctgctgagagctgggttccgtggccatgcacctgggactgccttgagaagcgtgagcaaaccgcccagagtagaagcgctagccaccatggattggggcacgctgcagacgatcctggggggtgtgaacaaacactccaccagcattggaaagatctggctcaccgtcctcttcatttttcgcattatgatcctcgttgtggctgcaaaggaggtgtggggagatgagcaggccgactttgtctgcaacaccctgcagccaggctgcaagaacgtgtgctacgatcactacttccccatctcccacatccggctatgggccctgcagctgatcttcgtgtccacgccagcgctcctagtggccatgcacgtggcctaccggagacatgagaagaagaggaagttcatcaagggggagataaagagtgaatttaaggacatcgaggagatcaaaacccagaaggtccgcatcgaaggctccctgtggtggacctacacaagcagcatcttcttccgggtcatcttcgaagccgccttcatgtacgtcttctatgtcatgtacgacggcttctccatgcagcggctggtgaagtgcaacgcctggccttgtcccaacactgtggactgctttgtgtcccggcccacggagaagactgtcttcacagtgttcatgattgcagtgtctggaatttgcatcctgctgaatgtcactgaattgtgttatttgctaattagatattgttctgggaagtcaaaaaagccagttggatcccgggctgactacaaagaccatgacggtgattataaagatcatgacatcgactacaaggatgacgatgacaagtaagaaatagacagcatgagagggatgaggcaacccgtgctcagctgtcaaggctcagtcgctagcatttcccaacacaaagattctgaccttaaatgcaaccatttgaaacccctgtaggcctcaggtgaaactccagatgccacaatggagctctgctcccctaaagcctcaaaacaaaggcctaattctatgcctgtcttaattttctttcacttaagttagttccactgagaccccaggctgttaggggttattggtgtaaggtactttcatattttaaacagaggatatcggcatttgtttctttctctgaggacaagagaaaaaagccaggttccacagaggacacagagaaggtttgggtgtcctcctggggttctttttgccaactttccccacgttaaaggtgaacattggttctttcatttgctttggaagttttaatctctaacagtggacaaagttaccagtgccttaaactctgttacactttttggaagtgaaaactttgtagtatgataggttattttgatgtaaagatgttctggataccattatatgttccccctgtttcagaggctcagattgtaatatgtaaatggtatgtcattcgctactatgatttaatttgaaatatggtcttttggttatgaatactttgcagcacagctgagaggctgtctgttgtattcattgtggtcatagcacctaacaacattgtagcctcaatcgagtgagacagactagaagttcctagtgatggcttatgatagcaaatggcctcatgtcaaatatttagatgtaattttgtgtaagaaatacagactggatgtaccaccaactactacctgtaatgacaggcctgtccaacacatctcccttttccatgactgtggtagccagcatcggaaagaacgctgatttaaagaggtcgcttgggaattttattgacacagtaccatttaatggggaggacaaaatggggcaggggagggagaagtttctgtcgttaaaaacagatttggaaagactggactctaaagtctgttgattaaagatgagctttgtctacttcaaaagtttgtttgcttaccccttcagcctccaattttttaagtgaaaatatagctaataacatgtgaaaagaatagaagctaaggtttagataaatattgagcagatctataggaagattgaacctgaatattgccattatgcttgacatggtttccaaaaaatggtactccacatatttcagtgagggtaagtattttcctgttgtcaagaatagcattgtaaaagcattttgtaataataaagaatagctttaatgatatgcttgtaactaaaataattttgtaatgtatcaaatacatttaaaacattaaaatataatctctataataagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggaagcttgaattcagctgacgtgcctcggaccgctaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaaccctatctcgggctattcttttgatttataagggattttgccgatttcggcctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgaacaataaaactgtctgcttacataaacagtaatacaaggggtgttatgagccatattcaacgggaaacgtcgaggccgcgattaaattccaacatggatgctgatttatatgggtataaatgggctcgcgataatgtcgggcaatcaggtgcgacaatctatcgcttgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccggaaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtctcgctcaggcgcaatcacgaatgaataacggtttggttgatgcgagtgattttgatgacgagcgtaatggctggcctgttgaacaagtctggaaagaaatgcataaacttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgt

SEQ ID NO: 39 - skipped

SEQ ID NO: 40 - DNA, 1316 bp, synthetic construct
  source 1..1316: mol_type = other DNA; note = PARM1 promoter
tgtacaggagatagtcagggaattagtaattttcaaagaggtgactttgaattcaaacttaaatatcatcttcagctgaaacaaagaaggggtgcagttatgaggaagtgaccaggtaaagcatggcaaacaaaggtaaagtttgttatgcgtatttaagtcagagccctctccattgataagagtttccagtaatttagtgccatccttttcttgctatagagttctcgtctctatctgagcacgcaaaaataacatgctttcttgctttcttgaagttgggcatggccattgacttgccttagcccatatttttctgtgaagtggtcttcaaaaacctatatttctgccatagagtcacttacttaacctgccctatttaaaggggctaatgcctgatagaatgtcgctgcataactccatctgtgtgtggtccctgcatccatgacaaccaaaacccagatgcagaaattgttcctaatcacatagattaccctagaaaccggaagggccttgaagtcaaaagcattcagagaacatgctgaacaaattgaatttgcagtttatctggccagggaggatggagaggggatgggcacttggtctgagtattttttgtttctcattccaacagaaattactagatttaccaaaaaatctacaagtggtagtgtgatagagtcaggcagaggaattgaccatagataaggtgctcaggactcctagagtcagcttctggtatgtgagaaagaagtgagaacagagcccatggcatatgaagaagatattacagaaaaaagaaagctgccttccacgcaaatcatttctttacaaaggcttgttaactcctgcagtgccaagaagctgaatgcagcggcagacatcctggttcgggccccaggaagctcagccgggtttaatgtggatgagggtttaatgatgtacacgcagaagtgttttgacaaatgaagaaggtcctcattcttggaacatgtgccggttctccgagggaactcctaaaaggctgtaagctcatgtaggaaaagctgagctagattcctaagggcagagatgtgctcacatttctttgcatccctagttcccagcacagtgcaaggcgctgcaaacatttgctgaacccagggtctcgtgtcttgactgtccagcagaggccgctctgggccggggctctcgggacctgagggctgagagaaggaaggccagggggtggcccagtcatcgccgcggggcccgggtgggaggggtttggcagcggcaggcgcggcggcggcggcggaggcggaggcggccccggg

SEQ ID NO: 41 - skipped

SEQ ID NO: 42 - DNA, 78 bp, synthetic construct
  source 1..78: mol_type = other DNA; note = Exemplary 3xFLAG tag sequence
ggatcccgggctgactacaaagaccatgacggtgattataaagatcatgacatcgactacaaggatgacgatgacaag

SEQ ID NO: 43 - DNA, 2208 bp, synthetic construct
  source 1..2208: mol_type = other DNA; note = Exemplary AAV Anc80 Capsid DNA Sequence
atggctgccgatggttatcttccagattggctcgaggacaacctctctgagggcattcgcgagtggtgggacttgaaacctggagccccgaaacccaaagccaaccagcaaaagcaggacgacggccggggtctggtgcttcctggctacaagtacctcggacccttcaacggactcgacaagggggagcccgtcaacgcggcggacgcagcggccctcgagcacgacaaggcctacgaccagcagctcaaagcgggtgacaatccgtacctgcggtataaccacgccgacgccgagtttcaggagcgtctgcaagaagatacgtcttttgggggcaacctcgggcgagcagtcttccaggccaagaagcgggttctcgaacctctcggtctggttgaggaaggcgctaagacggctcctggaaagaagagaccggtagagcaatcaccccaggaaccagactcctcttcgggcatcggcaagaaaggccagcagcccgcgaagaagagactcaactttgggcagacaggcgactcagagtcagtgcccgaccctcaaccactcggagaaccccccgcagccccctctggtgtgggatctaatacaatggcagcaggcggtggcgctccaatggcagacaataacgaaggcgccgacggagtgggtaacgcctcaggaaattggcattgcgattccacatggctgggcgacagagtcatcaccaccagcacccgaacctgggccctccccacctacaacaaccacctctacaagcaaatctccagccaatcgggagcaagcaccaacgacaacacctacttcggctacagcaccccctgggggtattttgactttaacagattccactgccacttctcaccacgtgactggcagcgactcatcaacaacaactggggattccggcccaagagactcaacttcaagctcttcaacatccaggtcaaggaggtcacgacgaatgatggcaccacgaccatcgccaataaccttaccagcacggttcaggtctttacggactcggaataccagctcccgtacgtcctcggctctgcgcaccagggctgcctgcctccgttcccggcggacgtcttcatgattcctcagtacgggtacctgactctgaacaatggcagtcaggccgtgggccgttcctccttctactgcctggagtactttccttctcaaatgctgagaacgggcaacaactttgagttcagctacacgtttgaggacgtgccttttcacagcagctacgcgcacagccaaagcctggaccggctgatgaaccccctcatcgaccagtacctgtactacctgtctcggactcagaccacgagtggtaccgcaggaaatcggacgttgcaattttctcaggccgggcctagtagcatggcgaatcaggccaaaaactggctacccgggccctgctaccggcagcaacgcgtctccaagacagcgaatcaaaataacaacagcaactttgcctggaccggtgccaccaagtatcatctgaatggcagagactctctggtaaatcccggtcccgctatggcaacccacaaggacgacgaagacaaattttttccgatgagcggagtcttaatatttgggaaacagggagctggaaatagcaacgtggaccttgacaacgttatgataaccagtgaggaagaaattaaaaccaccaacccagtggccacagaacagtacggcacggtggccactaacctgcaatcgtcaaacaccgctcctgctacagggaccgtcaacagtcaaggagccttacctggcatggtctggcagaaccgggacgtgtacctgcagggtcctatctgggccaagattcctcacacggacggacactttcatccctcgccgctgatgggaggctttggactgaaacacccgcctcctcagatcctgattaagaatacacctgttcccgcgaatcctccaactaccttcagtccagctaagtttgcgtcgttcatcacgcagtacagcaccggacaggtcagcgtggaaattgaatgggagctgcagaaagaaaacagcaaacgctggaacccagagattcaatacacttccaactacaacaaatctacaaatgtggactttgctgttgacacaaatggcgtttattctgagcctcgccccatcggcacccgttacctcacccgtaatctg

SEQ ID NO: 44 - protein, 736 aa, synthetic construct
  source 1..736: note = Exemplary AAV Anc80 Capsid Amino Acid Sequence
MAADGYLPDWLEDNLSEGIREWWDLKPGAPKPKANQQKQDDGRGLVLPGYKYLGPFNGLDKGEPVNAADAAALEHDKAYDQQLKAGDNPYLRYNHADAEFQERLQEDTSFGGNLGRAVFQAKKRVLEPLGLVEEGAKTAPGKKRPVEQSPQEPDSSSGIGKKGQQPAKKRLNFGQTGDSESVPDPQPLGEPPAAPSGVGSNTMAAGGGAPMADNNEGADGVGNASGNWHCDSTWLGDRVITTSTRTWALPTYNNHLYKQISSQSGASTNDNTYFGYSTPWGYFDFNRFHCHFSPRDWQRLINNNWGFRPKRLNFKLFNIQVKEVTTNDGTTTIANNLTSTVQVFTDSEYQLPYVLGSAHQGCLPPFPADVFMIPQYGYLTLNNGSQAVGRSSFYCLEYFPSQMLRTGNNFEFSYTFEDVPFHSSYAHSQSLDRLMNPLIDQYLYYLSRTQTTSGTAGNRTLQFSQAGPSSMANQAKNWLPGPCYRQQRVSKTANQNNNSNFAWTGATKYHLNGRDSLVNPGPAMATHKDDEDKFFPMSGVLIFGKQGAGNSNVDLDNVMITSEEEIKTTNPVATEQYGTVATNLQSSNTAPATGTVNSQGALPGMVWQNRDVYLQGPIWAKIPHTDGHFHPSPLMGGFGLKHPPPQILIKNTPVPANPPTTFSPAKFASFITQYSTGQVSVEIEWELQKENSKRWNPEIQYTSNYNKSTNVDFAVDTNGVYSEPRPIGTRYLTRNL

SEQ ID NO: 45 - DNA, 2989 bp, synthetic construct
  source 1..2989: mol_type = other DNA; note = Exemplary construct sequence
ttggccactccctctctgcgcgctcgctcgctcactgaggccgcccgggcaaagcccgggcgtcgggcgacctttggtcgcccggcctcagtgagcgagcgagcgcgcagagagggagtggccaactccatcactaggggttcctttgtcgacgcggccgcacgcgtgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatgggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgttgccttcgccccgtgccccgctccgcgccgcctcgcgccgcccgccccggctctgactgaccgcgttactcccacaggtgagcgggcgggacggcccttctcctccgggctgtaattagcgcttggtttaatgacggctcgtttcttttctgtggctgcgtgaaagccttaaagggctccgggagggccctttgtgcgggggggagcggctcggggggtgcgtgcgtgtgtgtgtgcgtggggagcgccgcgtgcggcccgcgctgcccggcggctgtgagcgctgcgggcgcggcgcggggctttgtgcgctccgcgtgtgcgcgaggggagcgcggccgggggcggtgccccgcggtgcgggggggctgcgaggggaacaaaggctgcgtgcggggtgtgtgcgtgggggggtgagcagggggtgtgggcgcggcggtcgggctgtaacccccccctgcacccccctccccgagttgctgagcacggcccggcttcgggtgcggggctccgtgcggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgaccggtgccaccatggattggggcacgctgcagacgatcctggggggtgtgaacaaacactccaccagcattggaaagatctggctcaccgtcctcttcatttttcgcattatgatcctcgttgtggctgcaaaggaggtgtggggagatgagcaggccgactttgtctgcaacaccctgcagccaggctgcaagaacgtgtgctacgatcactacttccccatctcccacatccggctatgggccctgcagctgatcttcgtgtccacgccagcgctcctagtggccatgcacgtggcctaccggagacatgagaagaagaggaagttcatcaagggggagataaagagtgaatttaaggacatcgaggagatcaaaacccagaaggtccgcatcgaaggctccctgtggtggacctacacaagcagcatcttcttccgggtcatcttcgaagccgccttcatgtacgtcttctatgtcatgtacgacggcttctccatgcagcggctggtgaagtgcaacgcctggccttgtcccaacactgtggactgctttgtgtcccggcccacggagaagactgtcttcacagtgttcatgattgcagtgtctggaatttgcatcctgctgaatgtcactgaattgtgttatttgctaattagatattgttctgggaagtcaaaaaagccagtttaagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggaagcttgaattcagctgacgtgcctcggaccgcctaggaggaacccctagtgatggagttggccactccctctctgcgcgctcgctcgctcactgaggccgggcgaccaaaggtcgcccgacgcccgggctttgcccgggcggcctcagtgagcgagcgagcgcgcagagagggagtggccaa

SEQ ID NO: 46 - DNA, 4421 bp, synthetic construct
  source 1..4421: mol_type = other DNA; note = Exemplary construct sequence
ttggccactccctctctgcgcgctcgctcgctcactgaggccgcccgggcaaagcccgggcgtcgggcgacctttggtcgcccggcctcagtgagcgagcgagcgcgcagagagggagtggccaactccatcactaggggttcctttgtcgacgcggccgcacgcgtgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatgggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgttgccttcgccccgtgccccgctccgcgccgcctcgcgccgcccgccccggctctgactgaccgcgttactcccacaggtgagcgggcgggacggcccttctcctccgggctgtaattagcgcttggtttaatgacggctcgtttcttttctgtggctgcgtgaaagccttaaagggctccgggagggccctttgtgcgggggggagcggctcggggggtgcgtgcgtgtgtgtgtgcgtggggagcgccgcgtgcggcccgcgctgcccggcggctgtgagcgctgcgggcgcggcgcggggctttgtgcgctccgcgtgtgcgcgaggggagcgcggccgggggcggtgccccgcggtgcgggggggctgcgaggggaacaaaggctgcgtgcggggtgtgtgcgtgggggggtgagcagggggtgtgggcgcggcggtcgggctgtaacccccccctgcacccccctccccgagttgctgagcacggcccggcttcgggtgcggggctccgtgcggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgaccggtgccaccatggattggggcacgctgcagacgatcctggggggtgtgaacaaacactccaccagcattggaaagatctggctcaccgtcctcttcatttttcgcattatgatcctcgttgtggctgcaaaggaggtgtggggagatgagcaggccgactttgtctgcaacaccctgcagccaggctgcaagaacgtgtgctacgatcactacttccccatctcccacatccggctatgggccctgcagctgatcttcgtgtccacgccagcgctcctagtggccatgcacgtggcctaccggagacatgagaagaagaggaagttcatcaagggggagataaagagtgaatttaaggacatcgaggagatcaaaacccagaaggtccgcatcgaaggctccctgtggtggacctacacaagcagcatcttcttccgggtcatcttcgaagccgccttcatgtacgtcttctatgtcatgtacgacggcttctccatgcagcggctggtgaagtgcaacgcctggccttgtcccaacactgtggactgctttgtgtcccggcccacggagaagactgtcttcacagtgttcatgattgcagtgtctggaatttgcatcctgctgaatgtcactgaattgtgttatttgctaattagatattgttctgggaagtcaaaaaagccagtttaagagctccgcattgcccagttgttagattaagaaatagacagcatgagagggatgaggcaacccgtgctcagctgtcaaggctcagtcgctagcatttcccaacacaaagattctgaccttaaatgcaaccatttgaaacccctgtaggcctcaggtgaaactccagatgccacaatggagcctctgctcccctaaagcctcaaaacaaaggcctaattctatgcctgtcttaattttctttcacttaagttagttccactgagaccccaggctgttaggggttattggtgtaaggtactttcatattttaaacagaggatatcggcatttgtttctttctctgaggacaagagaaaaaagccaggttccacagaggacacagagaaggtttgggtgtcctcctggggttctttttgccaactttccccacgttaaaggtgaacattggttctttcatttgctttggaagttttaatctctaacagtggacaaagttaccagtgccttaaactctgttacactttttggaagtgaaaactttgtagtatgataggttattttgatgtaaagatgttctggataccattatatgttccccctgtttcagaggctcagattgtaatatgtaaatggtatgtcattcgctactatgatttaatttgaaatatggtcttttggttatgaatactttgcagcacagctgagaggctgtctgttgtattcattgtggtcatagcacctaacaacattgtagcctcaatcgagtgagacagactagaagttcctagtgatggcttatgatagcaaatggcctcatgtcaaatatttagatgtaattttgtgtaagaaatacagactggatgtaccaccaactactacctgtaatgacaggcctgtccaacacatctcccttttccatgactgtggtagccagcatcggaaagaacgctgatttaaagaggtcgcttgggaattttattgacacagtaccatttaatggggaggacaaaatggggcaggggagggagaagtttctgtcgttaaaaacagatttggaaagactggactctaaagtctgttgattaaagatgagctttgtctacttcaaaagtttgtttgcttaccccttcagcctccaattttttaagtgaaaatatagctaataacatgtgaaaagaatagaagctaaggtttagataaatattgagcagatctataggaagattgaacctgaatattgccattatgcttgacatggtttccaaaaaatggtactccacatatttcagtgagggtaagtattttcctgttgtcaagaatagcattgtaaaagcattttgtaataataaagaatagctttaatgatatgcttgtaactaaaataattttgtaatgtatcaaatacatttaaaacattaaaatataatctctataataagctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggaagcttgaattcagctgacgtgcctcggaccgcctaggaggaacccctagtgatggagttggccactccctctctgcgcgctcgctcgctcactgaggccgggcgaccaaaggtcgcccgacgcccgggctttgcccgggcggcctcagtgagcgagcgagcgcgcagagagggagtggccaa

SEQ ID NO: 47 - DNA, 3938 bp, synthetic construct
  source 1..3938: mol_type = other DNA; note = Exemplary construct sequence
ttggccactccctctctgcgcgctcgctcgctcactgaggccgcccgggcaaagcccgggcgtcgggcgacctttggtcgcccggcctcagtgagcgagcgagcgcgcagagagggagtggccaactccatcactaggggttcctttgtcgacgcggccgcacgcgtaagcttcggtgaatttaaaacgtttggtggcagtgggtcaagtagccaggcggctgcgctagagtaccccgaagggacatcggcgacaccacaaacctcgcgctggcggctcgcccgcgcctttttcccctcccgcgcgcgcccggccccactcgcaccccgggcggtgccatcgcgtccacttccccggccgccccattccagctccggagctcggccgcagaaacgcccgctccagaaggcggcccccgccccccggcccaaggacgtgtgttggtccagccccccggttccccgagacccacgcggccgggcaaccgctctgggtctcgcggtccctccccgcgccaggttcctggccgggcagtccggggccggcgggctcacctgcgtcgggaggaagcgcggcggggccggggcgggggtctcggcgttggggtctctgcgctggggctcctgcgctcctaggcgggtcctgggccgggcgccgccgaggggctccgagtcggggagaggagcgcgcgggcgctgcggggccgcaacacctgtctcccgccgtggcgccttttaaccgcaccccacaccccgcctcttccctcggagactgggaaagttacggagggggcggcgccgcgggcggagcgcgcccggcctctgggtcctcagagcttcccgggtccgcgaacccccgaccgcccccgaaagccccgaaccccccaagtccccttcgaggtcccgatctcctagttcctttgagcccccatgagttccccaagtgcccccagcgccctgagtctcccccggttaccccgagcgccgcctcccccagccccttggcggcccgggtgaagcgggggcggctgagagtcgggaccccccaggaagcggcgccccagaccccggctccggcgctgtgccgtgggcggggttcagggatggctgtggtcgttgtcctctgtactccgcatagtgcgagaggacttggcatttatgagcgcttctttaattttttattgttagagaaacaggcattcctccaaggactgaagatctgttcgagtcgcggaggctgcgcgggcccgcgaggctctcgcagggggacctaggctgggtggcggggcagtgccctctggaatgggggttaacggtggccgaggagggggcgccgctggtgccggcgaagtccccgcttctttctcccctcaaaatctcaccaatccgaacgaacgccttctcgaatttccgattttattcaattactttcaacaatgtgccaaggactaaggttgggggcggtgggagagacaagcctcgtttttgccatggccggcaggggggtcccgccatctgcggagggtgccccccgcggcccccggcccagccaacttcctcctcttttcgcaactggggaactgcaaggaggtgactcctttcggggtgaggaggcccagacttttcagaaaggaaagagggcaggtaaaacctgccaagccccttcctgctcgatgcacacagcacgaaagggggaaactgataggattctgcggaagcttggcattccggtactgttggtaaagccaccagcaaaccgcccagagtagaagaccggtggccaccatggattggggcacgctgcagacgatcctggggggtgtgaacaaacactccaccagcattggaaagatctggctcaccgtcctcttcatttttcgcattatgatcctcgttgtggctgcaaaggaggtgtggggagatgagcaggccgactttgtctgcaacaccctgcagccaggctgcaagaacgtgtgctacgatcactacttccccatctcccacatccggctatgggccctgcagctgatcttcgtgtccacgccagcgctcctagtggccatgcacgtggcctaccggagacatgagaagaagaggaagttcatcaagggggagataaagagtgaatttaaggacatcgaggagatcaaaacccagaaggtccgcatcgaaggctccctgtggtggacctacacaagcagcatcttcttccgggtcatcttcgaagccgccttcatgtacgtcttctatgtcatgtacgacggcttctccatgcagcggctggtgaagtgcaacgcctggccttgtcccaacactgtggactgctttgtgtcccggcccacggagaagactgtcttcacagtgttcatgattgcagtgtctggaatttgcatcctgctgaatgtcactgaattgtgttatttgctaattagatattgttctgggaagtcaaaaaagccagtttaagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggaagcttgaattccttcttctggagtcttttctggaataattctgggagtgggctcagcctgcgggagagtaacatttttataacttgatagatgtagctgagatgcctcccagaggggagacccgcctctcctccggcagctgtgcacgtaggcttgttcccagcagcctggccagggtggtccacctggtgtttctcatcttctttccccggagcgctgactcctgcgcgtcctcttggaagactcttgacaggacgggtgttttatgggtgtgattcagtgtcctcttgcatcagttcaatgtggtggtgttcaatcaacccttgtagcgttagcaaaatttgctcaagtcattccgcaggaatgtctgtgtcttgcttccaagaaagcttgtaagtgccggcaacaggccaagcagctcacaaacctgaccacaagcctgtgagtaattgtggggcagcacttagcagtcttttattttcgacttattaaagtctcatcttggcctcaccttctccctggaaggtggcgtgggtgggaaccactgggtcagatctttttcacccttgccgtggagccagtttcctgttgcatgtgggggaagcaacatgtggtgaagagtatagaaaacgaaaacatgtgggtacagtatgtataagtggagggaacaaactcataattccaactagtttctcatgagagactcatgaatcattgtggtagttctcaatataaacttaatctaggccggatgtggtggctcacacctgtaatctcagcactctgggtggatcacttgaggtcaggagtttgagaccagtctgaccaacatggagaaaccccatcgctactaaaaatacaaaattatccagatgtggtggctcacacctgtaatcccagcactttgggaggctgaggcgggtggatcacttgaggtcaggagtttgagaccagcctgaccaacatggagaaactgtgtctctactaaaaatacaaaattagctgggcgtggtgacgcatgcctgtaatcccagctatttggaggccgaagcaggagctgacgtgcctcggaccgcctaggaggaacccctagtgatggagttggccactccctctctgcgcgctcgctcgctcactgaggccgggcgaccaaaggtcgcccgacgcccgggctttgcccgggcggcctcagtgagcgagcgagcgcgcagagagggagtggccaa

SEQ ID NO: 48 - DNA, 3916 bp, synthetic construct
  source 1..3916: mol_type = other DNA; note = Exemplary construct sequence
ttggccactccctctctgcgcgctcgctcgctcactgaggccgcccgggcaaagcccgggcgtcgggcgacctttggtcgcccggcctcagtgagcgagcgagcgcgcagagagggagtggccaactccatcactaggggttcctttgtcgacgcggccgcacgcgtaagcttcggtgaatttaaaacgtttggtggcagtgggtcaagtagccaggcggctgcgctagagtaccccgaagggacatcggcgacaccacaaacctcgcgctggcggctcgcccgcgcctttttcccctcccgcgcgcgcccggccccactcgcaccccgggcggtgccatcgcgtccacttccccggccgccccattccagctccggagctcggccgcagaaacgcccgctccagaaggcggcccccgccccccggcccaaggacgtgtgttggtccagccccccggttccccgagacccacgcggccgggcaaccgctctgggtctcgcggtccctccccgcgccaggttcctggccgggcagtccggggccggcgggctcacctgcgtcgggaggaagcgcggcggggccggggcgggggtctcggcgttggggtctctgcgctggggctcctgcgctcctaggcgggtcctgggccgggcgccgccgaggggctccgagtcggggagaggagcgcgcgggcgctgcggggccgcaacacctgtctcccgccgtggcgccttttaaccgcaccccacaccccgcctcttccctcggagactgggaaagttacggagggggcggcgccgcgggcggagcgcgcccggcctctgggtcctcagagcttcccgggtccgcgaacccccgaccgcccccgaaagccccgaaccccccaagtccccttcgaggtcccgatctcctagttcctttgagcccccatgagttccccaagtgcccccagcgccctgagtctcccccggttaccccgagcgccgcctcccccagccccttggcggcccgggtgaagcgggggcggctgagagtcgggaccccccaggaagcggcgccccagaccccggctccggcgctgtgccgtgggcggggttcagggatggctgtggtcgttgtcctctgtactccgcatagtgcgagaggacttggcatttatgagcgcttctttaattttttattgttagagaaacaggcattcctccaaggactgaagatctgttcgagtcgcggaggctgcgcgggcccgcgaggctctcgcagggggacctaggctgggtggcggggcagtgccctctggaatgggggttaacggtggccgaggagggggcgccgctggtgccggcgaagtccccgcttctttctcccctcaaaatctcaccaatccgaacgaacgccttctcgaatttccgattttattcaattactttcaacaatgtgccaaggactaaggttgggggcggtgggagagacaagcctcgtttttgccatggccggcaggggggtcccgccatctgcggagggtgccccccgcggcccccggcccagccaacttcctcctcttttcgcaactggggaactgcaaggaggtgactcctttcggggtgaggaggcccagacttttcagaaaggaaagagggcaggtaaaacctgccaagccccttcctgctcgatgcacacagcacgaaagggggaaactgataggattctgcggaagcttggcattccggtactgttggtaaagccaccagcaaaccgcccagagtagaagaccggtggccaccatggattggggcacgctgcagacgatcctggggggtgtgaacaaacactccaccagcattggaaagatctggctcaccgtcctcttcatttttcgcattatgatcctcgttgtggctgcaaaggaggtgtggggagatgagcaggccgactttgtctgcaacaccctgcagccaggctgcaagaacgtgtgctacgatcactacttccccatctcccacatccggctatgggccctgcagctgatcttcgtgtccacgccagcgctcctagtggccatgcacgtggcctaccggagacatgagaagaagaggaagttcatcaagggggagataaagagtgaatttaaggacatcgaggagatcaaaacccagaaggtccgcatcgaaggctccctgtggtggacctacacaagcagcatcttcttccgggtcatcttcgaagccgccttcatgtacgtcttctatgtcatgtacgacggcttctccatgcagcggctggtgaagtgcaacgcctggccttgtcccaacactgtggactgctttgtgtcccggcccacggagaagactgtcttcacagtgttcatgattgcagtgtctggaatttgcatcctgctgaatgtcactgaattgtgttatttgctaattagatattgttctgggaagtcaaaaaagccagtttaagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggaagcttgaattccctgtaaagccaattccaacccacttgtaattaagagaaaatcccacggttcctaattgaaagtcctttgttctatttcttgggtatttgtgttttaggccttatttttagatgcatcattaaagatttttaaagtcctttcaggcatcaggactgatgatgctgaatgatggagggttgtggataagtttttttgttttttttttaaccaggttaaaggctttcctgttatcctactatgcttaattaagagctgtatttcttaatatcattggtgcctgattagatttaacttttagatacagtctgtaagatttttgaaccagaaaaacctaaataacttatgactgttagcagtcatattctagaagaagcaaatgtactgaattcttatgtacctaggattttaagggagtacatacaaatctttcctcagtagcaggtactttatttttataacacacacatttaagctgagttaaatatgcagaactggttgtacttctttggcaggaaaagggaagcttaggatatcttgtgaccaactacctcttccttctcaaataactggcaaataacttcaggaaaatccagttatgttgtgtcatattgcaccccctaggaagtactggattcttagtcttgagtgacttttaaataaagctacctttttctctttcttacatcgcaagatcttcaaatgtaccattcccgcacagagagtccaaggtaaaaggactgaaaccaaactttgtttttgtaagtattttggtcagtgcaatgagttcagagaccaggaggttaatgattgtgaagtcttgtcaacagcaacaccgtgtatgacctgtggtgcttagatgttcagaaaccccaaggttaaaatgtccctgaccacatatcaggcaaaaggaatgtaaggaaaaccaacttaatccttttgtcaagaagtataaatgatgtatctttccaatcgggttgcattgacttttgggtccaaatagcttgtgtccacaggcatcttcagctgacgtgcctcggaccgcctaggaggaacccctagtgatggagttggccactccctctctgcgcgctcgctcgctcactgaggccgggcgaccaaaggtcgcccgacgcccgggctttgcccgggcggcctcagtgagcgagcgagcgcgcagagagggagtggccaa

SEQ ID NO: 49 - DNA, 2894 bp, synthetic construct
  source 1..2894: mol_type = other DNA; note = Exemplary construct sequence
ttggccactccctctctgcgcgctcgctcgctcactgaggccgcccgggcaaagcccgggcgtcgggcgacctttggtcgcccggcctcagtgagcgagcgagcgcgcagagagggagtggccaactccatcactaggggttcctttgtcgacgcggccgcacgcgtaagcttcggtgaatttaaaacgtttggtggcagtgggtcaagtagccaggcggctgcgctagagtaccccgaagggacatcggcgacaccacaaacctcgcgctggcggctcgcccgcgcctttttcccctcccgcgcgcgcccggccccactcgcaccccgggcggtgccatcgcgtccacttccccggccgccccattccagctccggagctcggccgcagaaacgcccgctccagaaggcggcccccgccccccggcccaaggacgtgtgttggtccagccccccggttccccgagacccacgcggccgggcaaccgctctgggtctcgcggtccctccccgcgccaggttcctggccgggcagtccggggccggcgggctcacctgcgtcgggaggaagcgcggcggggccggggcgggggtctcggcgttggggtctctgcgctggggctcctgcgctcctaggcgggtcctgggccgggcgccgccgaggggctccgagtcggggagaggagcgcgcgggcgctgcggggccgcaacacctgtctcccgccgtggcgccttttaaccgcaccccacaccccgcctcttccctcggagactgggaaagttacggagggggcggcgccgcgggcggagcgcgcccggcctctgggtcctcagagcttcccgggtccgcgaacccccgaccgcccccgaaagccccgaaccccccaagtccccttcgaggtcccgatctcctagttcctttgagcccccatgagttccccaagtgcccccagcgccctgagtctcccccggttaccccgagcgccgcctcccccagccccttggcggcccgggtgaagcgggggcggctgagagtcgggaccccccaggaagcggcgccccagaccccggctccggcgctgtgccgtgggcggggttcagggatggctgtggtcgttgtcctctgtactccgcatagtgcgagaggacttggcatttatgagcgcttctttaattttttattgttagagaaacaggcattcctccaaggactgaagatctgttcgagtcgcggaggctgcgcgggcccgcgaggctctcgcagggggacctaggctgggtggcggggcagtgccctctggaatgggggttaacggtggccgaggagggggcgccgctggtgccggcgaagtccccgcttctttctcccctcaaaatctcaccaatccgaacgaacgccttctcgaatttccgattttattcaattactttcaacaatgtgccaaggactaaggttgggggcggtgggagagacaagcctcgtttttgccatggccggcaggggggtcccgccatctgcggagggtgccccccgcggcccccggcccagccaacttcctcctcttttcgcaactggggaactgcaaggaggtgactcctttcggggtgaggaggcccagacttttcagaaaggaaagagggcaggtaaaacctgccaagccccttcctgctcgatgcacacagcacgaaagggggaaactgataggattctgcggaagcttggcattccggtactgttggtaaagccaccagcaaaccgcccagagtagaagaccggtggccaccatggattggggcacgctgcagacgatcctggggggtgtgaacaaacactccaccagcattggaaagatctggctcaccgtcctcttcatttttcgcattatgatcctcgttgtggctgcaaaggaggtgtggggagatgagcaggccgactttgtctgcaacaccctgcagccaggctgcaagaacgtgtgctacgatcactacttccccatctcccacatccggctatgggccctgcagctgatcttcgtgtccacgccagcgctcctagtggccatgcacgtggcctaccggagacatgagaagaagaggaagttcatcaagggggagataaagagtgaatttaaggacatcgaggagatcaaaacccagaaggtccgcatcgaaggctccctgtggtggacctacacaagcagcatcttcttccgggtcatcttcgaagccgccttcatgtacgtcttctatgtcatgtacgacggcttctccatgcagcggctggtgaagtgcaacgcctggccttgtcccaacactgtggactgctttgtgtcccggcccacggagaagactgtcttcacagtgttcatgattgcagtgtctggaatttgcatcctgctgaatgtcactgaattgtgttatttgctaattagatattgttctgggaagtcaaaaaagccagtttaagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggaagcttgaattcagctgacgtgcctcggaccgcctaggaggaacccctagtgatggagttggccactccctctctgcgcgctcgctcgctcactgaggccgggcgaccaaaggtcgcccgacgcccgggctttgcccgggcggcctcagtgagcgagcgagcgcgcagagagggagtggccaa

SEQ ID NO: 50 - DNA, 2987 bp, synthetic construct
  source 1..2987: mol_type = other DNA; note = Exemplary construct sequence
ttggccactccctctctgcgcgctcgctcgctcactgaggccgcccgggcaaagcccgggcgtcgggcgacctttggtcgcccggcctcagtgagcgagcgagcgcgcagagagggagtggccaactccatcactaggggttcctttgtcgacgcggccgcacgcgtgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatgggtcgaggtgagccccacgttctgcttcactctccccatctcccccccctccccacccccaattttgtatttatttattttttaattattttgtgcagcgatgggggcggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgttgccttcgccccgtgccccgctccgcgccgcctcgcgccgcccgccccggctctgactgaccgcgttactcccacaggtgagcgggcgggacggcccttctcctccgggctgtaattagcgcttggtttaatgacggctcgtttcttttctgtggctgcgtgaaagccttaaagggctccgggagggccctttgtgcgggggggagcggctcggggggtgcgtgcgtgtgtgtgtgcgtggggagcgccgcgtgcggcccgcgctgcccggcggctgtgagcgctgcgggcgcggcgcggggctttgtgcgctccgcgtgtgcgcgaggggagcgcggccgggggcggtgccccgcggtgcgggggggctgcgaggggaacaaaggctgcgtgcggggtgtgtgcgtgggggggtgagcagggggtgtgggcgcggcggtcgggctgtaacccccccctgcacccccctccccgagttgctgagcacggcccggcttcgggtgcggggctccgtgcggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgaccggtgccaccatggattggggcacgctgcagacgatcctggggggtgtgaacaaacactccaccagcattggaaagatctggctcaccgtcctcttcatttttcgcattatgatcctcgttgtggctgcaaaggaggtgtggggagatgagcaggccgactttgtctgcaacaccctgcagccaggctgcaagaacgtgtgctacgatcactacttccccatctcccacatccggctatgggccctgcagctgatcttcgtgtccacgccagcgctcctagtggccatgcacgtggcctaccggagacatgagaagaagaggaagttcatcaagggggagataaagagtgaatttaaggacatcgaggagatcaaaacccagaaggtccgcatcgaaggctccctgtggtggacctacacaagcagcatcttcttccgggtcatcttcgaagccgccttcatgtacgtcttctatgtcatgtacgacggcttctccatgcagcggctggtgaagtgcaacgcctggccttgtcccaacactgtggactgctttgtgtcccggcccacggagaagactgtcttcacagtgttcatgattgcagtgtctggaatttgcatcctgctgaatgtcactgaattgtgttatttgctaattagatattgttctgggaagtcaaaaaagccagtttaagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggaagcttgaattcagctgacgtgcctcggaccgcctaggaggaacccctagtgatggagttggccactccctctctgcgcgctcgctcgctcactgaggccgggcgaccaaaggtcgcccgacgcccgggctttgcccgggcggcctcagtgagcgagcgagcgcgcagagagggagtggccaa

SEQ ID NO: 51 - DNA, 4419 bp, synthetic construct
  source 1..4419: mol_type = other DNA; note = Exemplary construct sequence
ttggccactccctctctgcgcgctcgctcgctcactgaggccgcccgggcaaagcccgggcgtcgggcgacctttggtcgcccggcctcagtgagcgagcgagcgcgcagagagggagtggccaactccatcactaggggttcctttgtcgacgcggccgcacgcgtgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatgggtcgaggtgagccccacgttctgcttcactctccccatctcccccccctccccacccccaattttgtatttatttattttttaattattttgtgcagcgatgggggcggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgttgccttcgccccgtgccccgctccgcgccgcctcgcgccgcccgccccggctctgactgaccgcgttactcccacaggtgagcgggcgggacggcccttctcctccgggctgtaattagcgcttggtttaatgacggctcgtttcttttctgtggctgcgtgaaagccttaaagggctccgggagggccctttgtgcgggggggagcggctcggggggtgcgtgcgtgtgtgtgtgcgtggggagcgccgcgtgcggcccgcgctgcccggcggctgtgagcgctgcgggcgcggcgcggggctttgtgcgctccgcgtgtgcgcgaggggagcgcggccgggggcggtgccccgcggtgcgggggggctgcgaggggaacaaaggctgcgtgcggggtgtgtgcgtgggggggtgagcagggggtgtgggcgcggcggtcgggctgtaacccccccctgcacccccctccccgagttgctgagcacggcccggcttcgggtgcggggctccgtgcggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgaccggtgccaccatggattggggcacgctgcagacgatcctggggggtgtgaacaaacactccaccagcattggaaagatctggctcaccgtcctcttcatttttcgcattatgatcctcgttgtggctgcaaaggaggtgtggggagatgagcaggccgactttgtctgcaacaccctgcagccaggctgcaagaacgtgtgctacgatcactacttccccatctcccacatccggctatgggccctgcagctgatcttcgtgtccacgccagcgctcctagtggccatgcacgtggcctaccggagacatgagaagaagaggaagttcatcaagggggagataaagagtgaatttaaggacatcgaggagatcaaaacccagaaggtccgcatcgaaggctccctgtggtggacctacacaagcagcatcttcttccgggtcatcttcgaagccgccttcatgtacgtcttctatgtcatgtacgacggcttctccatgcagcggctggtgaagtgcaacgcctggccttgtcccaacactgtggactgctttgtgtcccggcccacggagaagactgtcttcacagtgttcatgattgcagtgtctggaatttgcatcctgctgaatgtcactgaattgtgttatttgctaattagatattgttctgggaagtcaaaaaagccagtttaagagctccgcattgcccagttgttagattaagaaatagacagcatgagagggatgaggcaacccgtgctcagctgtcaaggctcagtcgctagcatttcccaacacaaagattctgaccttaaatgcaaccatttgaaacccctgtaggcctcaggtgaaactccagatgccacaatggagcctctgctcccctaaagcctcaaaacaaaggcctaattctatgcctgtcttaattttctttcacttaagttagttccactgagaccccaggctgttaggggttattggtgtaaggtactttcatattttaaacagaggatatcggcatttgtttctttctctgaggacaagagaaaaaagccaggttccacagaggacacagagaaggtttgggtgtcctcctggggttctttttgccaactttccccacgttaaaggtgaacattggttctttcatttgctttggaagttttaatctctaacagtggacaaagttaccagtgccttaaactctgttacactttttggaagtgaaaactttgtagtatgataggttattttgatgtaaagatgttctggataccattatatgttccccctgtttcagaggctcagattgtaatatgtaaatggtatgtcattcgctactatgatttaatttgaaatatggtcttttggttatgaatactttgcagcacagctgagaggctgtctgttgtattcattgtggtcatagcacctaacaacattgtagcctcaatcgagtgagacagactagaagttcctagtgatggcttatgatagcaaatggcctcatgtcaaatatttagatgtaattttgtgtaagaaatacagactggatgtaccaccaactactacctgtaatgacaggcctgtccaacacatctcccttttccatgactgtggtagccagcatcggaaagaacgctgatttaaagaggtcgcttgggaattttattgacacagtaccatttaatggggaggacaaaatggggcaggggagggagaagtttctgtcgttaaaaacagatttggaaagactggactctaaagtctgttgattaaagatgagctttgtctacttcaaaagtttgtttgcttaccccttcagcctccaattttttaagtgaaaatatagctaataacatgtgaaaagaatagaagctaaggtttagataaatattgagcagatctataggaagattgaacctgaatattgccattatgcttgacatggtttccaaaaaatggtactccacatatttcagtgagggtaagtattttcctgttgtcaagaatagcattgtaaaagcattttgtaataataaagaatagctttaatgatatgcttgtaactaaaataattttgtaatgtatcaaatacatttaaaacattaaaatataatctctataataagctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggaagcttgaattcagctgacgtgcctcggaccgcctaggaggaacccctagtgatggagttggccactccctctctgcgcgctcgctcgctcactgaggccgggcgaccaaaggtcgcccgacgcccgggctttgcccgggcggcctcagtgagcgagcgagcgcgcagagagggagtggccaa

SEQ ID NO: 52 - DNA, 119 bp, synthetic construct
  source 1..119: mol_type = other DNA; note = Exemplary 5' AAV ITR
ctgcgcgctcgctcgctcactgaggccgcccgggcgtcgggcgacctttggtcgcccggcctcagtgagcgagcgagcgcgcagagagggagtggccaactccatcactaggggttcct

SEQ ID NO: 53 - DNA, 130 bp, synthetic construct
  source 1..130: mol_type = other DNA; note = Exemplary 3' AAV ITR
aggaacccctagtgatggagttggccactccctctctgcgcgctcgctcgctcactgaggccgggcgaccaaaggtcgcccgacgcccgggctttgcccgggcggcctcagtgagcgagcgagcgcgcag

SEQ ID NO: 54 - DNA, 7112 bp, synthetic construct
  source 1..7112: mol_type = other DNA; note = Exemplary Construct Sequence
cctgcaggcagctgcgcgctcgctcgctcactgaggccgcccgggcgtcgggcgacctttggtcgcccggcctcagtgagcgagcgagcgcgcagagagggagtggccaactccatcactaggggttcctgcggccgcacgcgtggtaagaaacttgcccgagtttacacagctagtaaatggttgcattagtcaggacagctagcctatattacaataacaaccctctcaaatcctaatggcttaaaacaacagaggtttaatttatactcattagctgttcaaggcaggaggctctattctctaatccatacagtcactcaggatccaggctggtggagaccctgccatattgtagcctcaccatttaaaacatgaagaagatagaaagtgaggagtcatgtaggttttgttccgttgcctcaggctaggagtgacaggtcacttcatctcactcacagctcactgcccacaactagtcacttgtgactgtgcgagttaagcttctgtgtgtgaaggaaggaaaagagaatgggataaaggtgaacatcagcaggctctaccacagtagtttgaaccaagacttgagcctaggtcatgtggcttcagaatctttgctcttaatcacactaaacagcctctgtaagtcatctttccttcatccagtgcctaagaacatgcagtccaatgccctcatccttcagaagaacttgagtgaactcagagaaattgagtagagtgccacagcatgcccaaggccacacaccctgaggttggcagtaggtcctgagttagagttgtcatttcttggctcccctggtagtagtggaaaggtaaggttttgacatactagttggatgaccacgggcaggtcacttaaattgtctaagcatcgtttgacccttgtaagaattaaatgaaatagcacctgtaaaagtgtctgcacggacttactgctgttagttttgttcctttcttcctgttgtcactgcacttccctgcctgttacccaggccatgcagaccagccaggccttcgacttacagtgcggataagattccaaatctccacggctggtttccatgctttcttccaggcttctgaggaccctgtgctctggtttcttctatttcttttctattacttttctgttactcttgagcacacttgctggaagcaatatgcatccagttctccctctcttgcctcattacactttgcagaacaactccaatcccttccaaccaagtagtccctttgaatttcttgtcacccaaggaatctctctgacaggggtctttgttagggtcacaccccaggagatggttgattatggctgagtccagcctggaatgatgggggttgggggcagcttgggtagatgactcagtaaatcaaacagaacaatgaaaggaggtcatgcttgtccatctgcattattgaagacagccataaatggccttaccccagagcgggtctgtcacacctggagagctgatctgacctctccaagacccctgcaactgagtgttctgggatctgtcctgcaacaagtgcctcgagatttgtaggtgggggcccagagggagggggtctgcagacgaagggggcaggttttgcggggcacttagggttctcataggttgtagtcacgagctccaagctctgaggacccagaggccgggcgcgctccgcccgcggcgccgccccctccgtaactttcccagtctccgagggaagaggcggggtgtggggtgcggttaaaaggcgccacggcgggagacaggtctcaccggtcacaacctgttgcggccccgcagcgcccgcgcgctcctctccccgactcggagcccctcggcggcgcccggcccaggacccgcctaggagcgcaggagccccagcgcagagaccccaacgccgagacccccgccccggccccgccgcgcttcctcccgacgcagtttaggacccttgttcgcgaagaggtggtgtgcggctgagacccgcgtcctcaggacggttccatcagtgcctcgatcctgccccactggaggaggaaggcagcccgaacagcgctcacctaactaacagctgctgagagctgggttccgtggccatgcacctgggactgccttgagaagcgtgagcaaaccgcccagagtagaagcgctagccaccatggattggggcacgctgcagacgatcctggggggtgtgaacaaacactccaccagcattggaaagatctggctcaccgtcctcttcatttttcgcattatgatcctcgttgtggctgcaaaggaggtgtggggagatgagcaggccgactttgtctgcaacaccctgcagccaggctgcaagaacgtgtgctacgatcactacttccccatctcccacatccggctatgggccctgcagctgatcttcgtgtccacgccagcgctcctagtggccatgcacgtggcctaccggagacatgagaagaagaggaagttcatcaagggggagataaagagtgaatttaaggacatcgaggagatcaaaacccagaaggtccgcatcgaaggctccctgtggtggacctacacaagcagcatcttcttccgggtcatcttcgaagccgccttcatgtacgtcttctatgtcatgtacgacggcttctccatgcagcggctggtgaagtgcaacgcctggccttgtcccaacactgtggactgctttgtgtcccggcccacggagaagactgtcttcacagtgttcatgattgcagtgtctggaatttgcatcctgctgaatgtcactgaattgtgttatttgctaattagatattgttctgggaagtcaaaaaagccagttggatcccgggctgactacaaagaccatgacggtgattataaagatcatgacatcgactacaaggatgacgatgacaagtaagaaatagacagcatgagagggatgaggcaacccgtgctcagctgtcaaggctcagtcgctagcatttcccaacacaaagattctgaccttaaatgcaaccatttgaaacccctgtaggcctcaggtgaaactccagatgccacaatggagctctgctcccctaaagcctcaaaacaaaggcctaattctatgcctgtcttaattttctttcacttaagttagttccactgagaccccaggctgttaggggttattggtgtaaggtactttcatattttaaacagaggatatcggcatttgtttctttctctgaggacaagagaaaaaagccaggttccacagaggacacagagaaggtttgggtgtcctcctggggttctttttgccaactttccccacgttaaaggtgaacattggttctttcatttgctttggaagttttaatctctaacagtggacaaagttaccagtgccttaaactctgttacactttttggaagtgaaaactttgtagtatgataggttattttgatgtaaagatgttctggataccattatatgttccccctgtttcagaggctcagattgtaatatgtaaatggtatgtcattcgctactatgatttaatttgaaatatggtcttttggttatgaatactttgcagcacagctgagaggctgtctgttgtattcattgtggtcatagcacctaacaacattgtagcctcaatcgagtgagacagactagaagttcctagtgatggcttatgatagcaaatggcctcatgtcaaatatttagatgtaattttgtgtaagaaatacagactggatgtaccaccaactactacctgtaatgacaggcctgtccaacacatctcccttttccatgactgtggtagccagcatcggaaagaacgctgatttaaagaggtcgcttgggaattttattgacacagtaccatttaatggggaggacaaaatggggcaggggagggagaagtttctgtcgttaaaaacagatttggaaagactggactctaaagtctgttgattaaagatgagctttgtctacttcaaaagtttgtttgcttaccccttcagcctccaattttttaagtgaaaatatagctaataacatgtgaaaagaatagaagctaaggtttagataaatattgagcagatctataggaagattgaacctgaatattgccattatgcttgacatggtttccaaaaaatggtactccacatatttcagtgagggtaagtattttcctgttgtcaagaatagcattgtaaaagcattttgtaataataaagaatagctttaatgatatgcttgtaactaaaataattttgtaatgtatcaaatacatttaaaacattaaaatataatctctataataagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggaagcttgaattcagctgacgtgcctcggaccgctaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaaccctatctcgggctattcttttgatttataagggattttgccgatttcggcctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgaacaataaaactgtctgcttacataaacagtaatacaaggggtgttatgagccatattcaacgggaaacgtcgaggccgcgattaaattccaacatggatgctgatttatatgggtataaatgggctcgcgataatgtcgggcaatcaggtgcgacaatctatcgcttgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccggaaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtctcgctcaggcgcaatcacgaatgaataacggtttggttgatgcgagtgattttgatgacgagcgtaatggctggcctgttgaacaagtctggaaagaaatgcataaacttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgt

SEQ ID NO: 55-56 - skipped (2 entries)

SEQ ID NO: 57 - DNA, 1513 bp, synthetic construct
  source 1..1513: mol_type = other DNA; note = Exemplary IGFBP2 promoter
aagaaacttgcccgagtttacacagctagtaaatggttgcattagtcaggacagctagcctatattacaataacaaccctctcaaatcctaatggcttaaaacaacagaggtttaatttatactcattagctgttcaaggcaggaggctctattctctaatccatacagtcactcaggatccaggctggtggagaccctgccatattgtagcctcaccatttaaaacatgaagaagatagaaagtgaggagtcatgtaggttttgttccgttgcctcaggctaggagtgacaggtcacttcatctcactcacagctcactgcccacaactagtcacttgtgactgtgcgagttaagcttctgtgtgtgaaggaaggaaaagagaatgggataaaggtgaacatcagcaggctctaccacagtagtttgaaccaagacttgagcctaggtcatgtggcttcagaatctttgctcttaatcacactaaacagcctctgtaagtcatctttccttcatccagtgcctaagaacatgcagtccaatgccctcatccttcagaagaacttgagtgaactcagagaaattgagtagagtgccacagcatgcccaaggccacacaccctgaggttggcagtaggtcctgagttagagttgtcatttcttggctcccctggtagtagtggaaaggtaaggttttgacatactagttggatgaccacgggcaggtcacttaaattgtctaagcatcgtttgacccttgtaagaattaaatgaaatagcacctgtaaaagtgtctgcacggacttactgctgttagttttgttcctttcttcctgttgtcactgcacttccctgcctgttacccaggccatgcagaccagccaggccttcgacttacagtgcggataagattccaaatctccacggctggtttccatgctttcttccaggcttctgaggaccctgtgctctggtttcttctatttcttttctattacttttctgttactcttgagcacacttgctggaagcaatatgcatccagttctccctctcttgcctcattacactttgcagaacaactccaatcccttccaaccaagtagtccctttgaatttcttgtcacccaaggaatctctctgacaggggtctttgttagggtcacaccccaggagatggttgattatggctgagtccagcctggaatgatgggggttgggggcagcttgggtagatgactcagtaaatcaaacagaacaatgaaaggaggtcatgcttgtccatctgcattattgaagacagccataaatggccttaccccagagcgggtctgtcacacctggagagctgatctgacctctccaagacccctgcaactgagtgttctgggatctgtcctgcaacaagtgcctcgagatttgtaggtgggggcccagagggagggggtctgcagacgaagggggcaggttttgcggggcacttagggttctcataggttgtagtcacgagctcc

SEQ ID NO: 58 - DNA, 24 bp, synthetic construct
  source 1..24: mol_type = other DNA; note = Exemplary Taqman probe for quantification of constructs
tctggctcaccgtcctcttcattt

SEQ ID NO: 59 - DNA, 20 bp, synthetic construct
  source 1..20: mol_type = other DNA; note = Exemplary forward qPCR primer for quantification of constructs
caaacactccaccagcattg

SEQ ID NO: 60 - DNA, 20 bp, synthetic construct
  source 1..20: mol_type = other DNA; note = Exemplary reverse qPCR primer for quantification of constructs
cagccacaacgaggatcata

SEQ ID NO: 61 - DNA, 6787 bp, synthetic construct
  source 1..6787: mol_type = other DNA; note = Exemplary Construct Sequence
cctgcaggcagctgcgcgctcgctcgctcactgaggccgcccgggcgtcgggcgacctttggtcgcccggcctcagtgagcgagcgagcgcgcagagagggagtggccaactccatcactaggggttcctgcggccgcacgcgtggtccacaggtaactccgtcggcgtccacaggggggcaggagataccatactgcacagttgtacgtcttccatctgtttggtgtagaaaaatctaaccactacaagaatgccacgggcactgtggcagacagaagcagcgctacgccgcatcgcctttcagcgtgcaggcccaggaatgagcgaggcagtgggcggggaagacaggcacggggaatctggggacagataaaggaaactcgtgatggggcgaggctgggctgaagagaaacagattggggtagagctgcaaagggaggggtccactggaaggcgaggggggaggccgggaagagagagggtgggaaggcagtgtgagatgggagggcagtgtgagaagaaaagcaggctggggaagagggattggaatgcagaaggaacttggggaaggaggaagtcctgcaggcgggagggaaagaagagagggggagcagctaaagtctgcgtcagaagaggttggggactgcgagaggagaggctggggcctgcaggggagcgcagcagcttttagcatcgatccaaactctaaagactcgtggcctttgcctgacctcgagggtcgggaatagacgcctgtctttgtggagagcgatacccaaccgagaaaatggggctgttccgagctgggccctgcgcctggcccagggcgaggcttctctggctccgggctggcccctgaggggcagcacgcagcctgcagcagaggcgcctgctccaagctgtctcttgggggcgccgccgccgcttccctcctccggggccgctcgctcccaggaaagtggaggcggctggcgaggaccgagagccggggccgcgctgcggagggaccacacctccgggagttcgagggggaccctggcgcggcgggccagcctttcgggccggcagcgcccgccttcccccggtcagcgcttgcggcccgcgccgcgcgcaccgcccggcaaccccgcgcgcgtcccgcgggggcgctgcgtcttcctgccacaccggcgcaccgcggcccctctcccccacacctccggcccgcaccacccggctctcctcccaccctccccacccctcctctgccctccctccccattcctcccctcccggcgaggggcgggagggggcgtggcggggccggggtttgtgtggctgggacccggctcctcaagctctgaggacccagaggccgggcgcgctccgcccgcggcgccgccccctccgtaactttcccagtctccgagggaagaggcggggtgtggggtgcggttaaaaggcgccacggcgggagacaggtctcaccggtgtgtcaccgttgcggccccgcagcgcccgcgcgctcctctccccgactcggagcccctcggcggcgcccggcccaggacccgcctaggagcgcaggagccccagcgcagagaccccaacgccgagacccccgccccggccccgccgcgcttcctcccgacgcagtttaggacccttgttcgcgaagaggtggtgtgcggctgagacccgcgtcctcaggacggttccatcagtgcctcgatcctgccccactggaggaggaaggcagcccgaacagcgctcacctaactaacagctgctgagagctgggttccgtggccatgcacctgggactgccttgagaagcgtgagcaaaccgcccagagtagaagcgctagccaccatggattggggcacgctgcagacgatcctggggggtgtgaacaaacactccaccagcattggaaagatctggctcaccgtcctcttcatttttcgcattatgatcctcgttgtggctgcaaaggaggtgtggggagatgagcaggccgactttgtctgcaacaccctgcagccaggctgcaagaacgtgtgctacgatcactacttccccatctcccacatccggctatgggccctgcagctgatcttcgtgtccacgccagcgctcctagtggccatgcacgtggcctaccggagacatgagaagaagaggaagttcatcaagggggagataaagagtgaatttaaggacatcgaggagatcaaaacccagaaggtccgcatcgaaggctccctgtggtggacctacacaagcagcatcttcttccgggtcatcttcgaagccgccttcatgtacgtcttctatgtcatgtacgacggcttctccatgcagcggctggtgaagtgcaacgcctggccttgtcccaacactgtggactgctttgtgtcccggcccacggagaagactgtcttcacagtgttcatgattgcagtgtctggaatttgcatcctgctgaatgtcactgaattgtgttatttgctaattagatattgttctgggaagtcaaaaaagccagttggatcccgggctgactacaaagaccatgacggtgattataaagatcatgacatcgactacaaggatgacgatgacaagtaagaaatagacagcatgagagggatgaggcaacccgtgctcagctgtcaaggctcagtcgctagcatttcccaacacaaagattctgaccttaaatgcaaccatttgaaacccctgtaggcctcaggtgaaactccagatgccacaatggagctctgctcccctaaagcctcaaaacaaaggcctaattctatgcctgtcttaattttctttcacttaagttagttccactgagaccccaggctgttaggggttattggtgtaaggtactttcatattttaaacagaggatatcggcatttgtttctttctctgaggacaagagaaaaaagccaggttccacagaggacacagagaaggtttgggtgtcctcctggggttctttttgccaactttccccacgttaaaggtgaacattggttctttcatttgctttggaagttttaatctctaacagtggacaaagttaccagtgccttaaactctgttacactttttggaagtgaaaactttgtagtatgataggttattttgatgtaaagatgttctggataccattatatgttccccctgtttcagaggctcagattgtaatatgtaaatggtatgtcattcgctactatgatttaatttgaaatatggtcttttggttatgaatactttgcagcacagctgagaggctgtctgttgtattcattgtggtcatagcacctaacaacattgtagcctcaatcgagtgagacagactagaagttcctagtgatggcttatgatagcaaatggcctcatgtcaaatatttagatgtaattttgtgtaagaaatacagactggatgtaccaccaactactacctgtaatgacaggcctgtccaacacatctcccttttccatgactgtggtagccagcatcggaaagaacgctgatttaaagaggtcgcttgggaattttattgacacagtaccatttaatggggaggacaaaatggggcaggggagggagaagtttctgtcgttaaaaacagatttggaaagactggactctaaagtctgttgattaaagatgagctttgtctacttcaaaagtttgtttgcttaccccttcagcctccaattttttaagtgaaaatatagctaataacatgtgaaaagaatagaagctaaggtttagataaatattgagcagatctataggaagattgaacctgaatattgccattatgcttgacatggtttccaaaaaatggtactccacatatttcagtgagggtaagtattttcctgttgtcaagaatagcattgtaaaagcattttgtaataataaagaatagctttaatgatatgcttgtaactaaaataattttgtaatgtatcaaatacatttaaaacattaaaatataatctctataataagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggaagcttgaattcagctgacgtgcctcggaccgctaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaaccctatctcgggctattcttttgatttataagggattttgccgatttcggcctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgaacaataaaactgtctgcttacataaacagtaatacaaggggtgttatgagccatattcaacgggaaacgtcgaggccgcgattaaattccaacatggatgctgatttatatgggtataaatgggctcgcgataatgtcgggcaatcaggtgcgacaatctatcgcttgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccggaaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtctcgctcaggcgcaatcacgaatgaataacggtttggttgatgcgagtgattttgatgacgagcgtaatggctggcctgttgaacaagtctggaaagaaatgcataaacttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgt

SEQ ID NO: 62 - skipped

SEQ ID NO: 63 - DNA, 382 bp, synthetic construct
  source 1..382: mol_type = other DNA; note = Exemplary CMV enhancer
gacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggt

SEQ ID NO: 64 - DNA, 245 bp, synthetic construct
  source 1..245: mol_type = other DNA; note = Exemplary chimeric intron
ggagtcgctgcgttgccttcgccccgtgccccgctccgcgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagctagagcctctgctaaccatgttcatgccttcttctttttcctacag

SEQ ID NO: 65 - DNA, 1044 bp, synthetic construct
  source 1..1044: mol_type = other DNA; note = Exemplary GJB2 enhancer
cttcttctggagtcttttctggaataattctgggagtgggctcagcctgcgggagagtaacatttttataacttgatagatgtagctgagatgcctcccagaggggagacccgcctctcctccggcagctgtgcacgtaggcttgttcccagcagcctggccagggtggtccacctggtgtttctcatcttctttccccggagcgctgactcctgcgcgtcctcttggaagactcttgacaggacgggtgttttatgggtgtgattcagtgtcctcttgcatcagttcaatgtggtggtgttcaatcaacccttgtagcgttagcaaaatttgctcaagtcattccgcaggaatgtctgtgtcttgcttccaagaaagcttgtaagtgccggcaacaggccaagcagctcacaaacctgaccacaagcctgtgagtaattgtggggcagcacttagcagtcttttattttcgacttattaaagtctcatcttggcctcaccttctccctggaaggtggcgtgggtgggaaccactgggtcagatctttttcacccttgccgtggagccagtttcctgttgcatgtgggggaagcaacatgtggtgaagagtatagaaaacgaaaacatgtgggtacagtatgtataagtggagggaacaaactcataattccaactagtttctcatgagagactcatgaatcattgtggtagttctcaatataaacttaatctaggccggatgtggtggctcacacctgtaatctcagcactctgggtggatcacttgaggtcaggagtttgagaccagtctgaccaacatggagaaaccccatcgctactaaaaatacaaaattatccagatgtggtggctcacacctgtaatcccagcactttgggaggctgaggcgggtggatcacttgaggtcaggagtttgagaccagcctgaccaacatggagaaactgtgtctctactaaaaatacaaaattagctgggcgtggtgacgcatgcctgtaatcccagctatttggaggccgaagcagg

SEQ ID NO: 66 - DNA, 362 bp, synthetic construct
  source 1..362: mol_type = other DNA; note = Exemplary 5' UTR Sequence
gttgcggccccgcagcgcccgcgcgctcctctccccgactcggagcccctcggcggcgcccggcccaggacccgcctaggagcgcaggagccccagcgcagagaccccaacgccgagacccccgccccggccccgccgcgcttcctcccgacgcagtttaggacccttgttcgcgaagaggtggtgtgcggctgagacccgcgtcctcaggacggttccatcagtgcctcgatcctgccccactggaggaggaaggcagcccgaacagcgctcacctaactaacagctgctgagagctgggttccgtggccatgcacctgggactgccttgagaagcgtgagcaaaccgcccagagtagaag

SEQ ID NO: 67 - DNA, 1407 bp, synthetic construct
  source 1..1407: mol_type = other DNA; note = Exemplary 3' UTR Sequence
gaaatagacagcatgagagggatgaggcaacccgtgctcagctgtcaaggctcagtcgctagcatttcccaacacaaagattctgaccttaaatgcaaccatttgaaacccctgtaggcctcaggtgaaactccagatgccacaatggagctctgctcccctaaagcctcaaaacaaaggcctaattctatgcctgtcttaattttctttcacttaagttagttccactgagaccccaggctgttaggggttattggtgtaaggtactttcatattttaaacagaggatatcggcatttgtttctttctctgaggacaagagaaaaaagccaggttccacagaggacacagagaaggtttgggtgtcctcctggggttctttttgccaactttccccacgttaaaggtgaacattggttctttcatttgctttggaagttttaatctctaacagtggacaaagttaccagtgccttaaactctgttacactttttggaagtgaaaactttgtagtatgataggttattttgatgtaaagatgttctggataccattatatgttccccctgtttcagaggctcagattgtaatatgtaaatggtatgtcattcgctactatgatttaatttgaaatatggtcttttggttatgaatactttgcagcacagctgagaggctgtctgttgtattcattgtggtcatagcacctaacaacattgtagcctcaatcgagtgagacagactagaagttcctagtgatggcttatgatagcaaatggcctcatgtcaaatatttagatgtaattttgtgtaagaaatacagactggatgtaccaccaactactacctgtaatgacaggcctgtccaacacatctcccttttccatgactgtggtagccagcatcggaaagaacgctgatttaaagaggtcgcttgggaattttattgacacagtaccatttaatggggaggacaaaatggggcaggggagggagaagtttctgtcgttaaaaacagatttggaaagactggactctaaagtctgttgattaaagatgagctttgtctacttcaaaagtttgtttgcttaccccttcagcctccaattttttaagtgaaaatatagctaataacatgtgaaaagaatagaagctaaggtttagataaatattgagcagatctataggaagattgaacctgaatattgccattatgcttgacatggtttccaaaaaatggtactccacatatttcagtgagggtaagtattttcctgttgtcaagaatagcattgtaaaagcattttgtaataataaagaatagctttaatgatatgcttgtaactaaaataattttgtaatgtatcaaatacatttaaaacattaaaatataatctctataataa

SEQ ID NO: 68 - DNA, 1248 bp, synthetic construct
  source 1..1248: mol_type = other DNA; note = Exemplary 3' UTR Sequence
cgcattgcccagttgttagattaagaaatagacagcatgagagggatgaggcaacccgtgctcagctgtcaaggctcagtcgctagcatttcccaacacaaagattctgaccttaaatgcaaccatttgaaacccctgtaggcctcaggtgaaactccagatgccacaatggagcctctgctcccctaaagcctcaaaacaaaggcctaattctatgcctgtcttaattttctttcacttaagttagttccactgagaccccaggctgttaggggttattggtgtaaggtactttcatattttaaacagaggatatcggcatttgtttctttctctgaggacaagagaaaaaagccaggttccacagaggacacagagaaggtttgggtgtcctcctggggttctttttgccaactttccccacgttaaaggtgaacattggttctttcatttgctttggaagttttaatctctaacagtggacaaagttaccagtgccttaaactctgttacactttttggaagtgaaaactttgtagtatgataggttattttgatgtaaagatgttctggataccattatatgttccccctgtttcagaggctcagattgtaatatgtaaatggtatgtcattcgctactatgatttaatttgaaatatggtcttttggttatgaatactttgcagcacagctgagaggctgtctgttgtattcattgtggtcatagcacctaacaacattgtagcctcaatcgagtgagacagactagaagttcctagtgatggcttatgatagcaaatggcctcatgtcaaatatttagatgtaattttgtgtaagaaatacagactggatgtaccaccaactactacctgtaatgacaggcctgtccaacacatctcccttttccatgactgtggtagccagcatcggaaagaacgctgatttaaagaggtcgcttgggaattttattgacacagtaccatttaatggggaggacaaaatggggcaggggagggagaagtttctgtcgttaaaaacagatttggaaagactggactctaaagtctgttgattaaagatgagctttgtctacttcaaaagtttgtttgcttaccccttcagcctccaattttttaagtgaaaatatagctaataacatgtgaaaagaatagaagctaaggtttagataaatattgagcagatctataggaagattgaacctgaatattgccattatgcttgac

SEQ ID NO: 69 - DNA, 1372 bp, synthetic construct
  source 1..1372: mol_type = other DNA; note = Exemplary 3' UTR Sequence
gagctcagtgtgagttctaccattgccaaactcgagcagtgaattctaccagtgccataggatccagtgtgagttctaccattgccaaaggtacccagtgaattctaccagtgccatagttaaccgcattgcccagttgttagattaagaaatagacagcatgagagggatgaggcaacccgtgctcagctgtcaaggctcagtcgctagcatttcccaacacaaagattctgaccttaaatgcaaccatttgaaacccctgtaggcctcaggtgaaactccagatgccacaatggagcctctgctcccctaaagcctcaaaacaaaggcctaattctatgcctgtcttaattttctttcacttaagttagttccactgagaccccaggctgttaggggttattggtgtaaggtactttcatattttaaacagaggatatcggcatttgtttctttctctgaggacaagagaaaaaagccaggttccacagaggacacagagaaggtttgggtgtcctcctggggttctttttgccaactttccccacgttaaaggtgaacattggttctttcatttgctttggaagttttaatctctaacagtggacaaagttaccagtgccttaaactctgttacactttttggaagtgaaaactttgtagtatgataggttattttgatgtaaagatgttctggataccattatatgttccccctgtttcagaggctcagattgtaatatgtaaatggtatgtcattcgctactatgatttaatttgaaatatggtcttttggttatgaatactttgcagcacagctgagaggctgtctgttgtattcattgtggtcatagcacctaacaacattgtagcctcaatcgagtgagacagactagaagttcctagtgatggcttatgatagcaaatggcctcatgtcaaatatttagatgtaattttgtgtaagaaatacagactggatgtaccaccaactactacctgtaatgacaggcctgtccaacacatctcccttttccatgactgtggtagccagcatcggaaagaacgctgatttaaagaggtcgcttgggaattttattgacacagtaccatttaatggggaggacaaaatggggcaggggagggagaagtttctgtcgttaaaaacagatttggaaagactggactctaaagtctgttgattaaagatgagctttgtctacttcaaaagtttgtttgcttaccccttcagcctccaattttttaagtgaaaatatagctaataacatgtgaaaagaatagaagctaaggtttagataaatattgagcagatctataggaagattgaacctgaatattgccattatgcttgac

SEQ ID NO: 70 - DNA, 14 bp, synthetic construct
  source 1..14: mol_type = other DNA; note = Exemplary cloning site J
gcggccgcacgcgt

SEQ ID NO: 71 - DNA, 17 bp, synthetic construct
  source 1..17: mol_type = other DNA; note = Exemplary cloning site K
gcggccgcacgcgtggt

SEQ ID NO: 72 - DNA, 33 bp, synthetic construct
  source 1..33: mol_type = other DNA; note = Exemplary cloning site L
ctcctgggcaacgtgctggttattgtgaccggt

SEQ ID NO: 73 - DNA, 11 bp, synthetic construct
  source 1..11: mol_type = other DNA; note = Exemplary cloning site M
cgctagccacc

SEQ ID NO: 74 - DNA, 17 bp, synthetic construct
  source 1..17: mol_type = other DNA; note = Exemplary cloning site N
accggtcgctagccacc

SEQ ID NO: 75 - DNA, 21 bp, synthetic construct
  source 1..21: mol_type = other DNA; note = Exemplary cloning site O
gagctcgctgatcagcctcga

SEQ ID NO: 76 - DNA, 34 bp, synthetic construct
  source 1..34: mol_type = other DNA; note = Exemplary cloning site P
aagcttgaattcagctgacgtgcctcggaccgct

SEQ ID NO: 77 - DNA, 12 bp, synthetic construct
  source 1..12: mol_type = other DNA; note = Exemplary linker sequence
ggatcccgggct

SEQ ID NO: 78 - DNA, 24 bp, synthetic construct
  source 1..24: mol_type = other DNA; note = Exemplary mRNA destabilizing domain sequence
agtgtgagttctaccattgccaaa

SEQ ID NO: 79 - DNA, 22 bp, synthetic construct
  source 1..22: mol_type = other DNA; note = Exemplary mRNA destabilizing domain sequence
agtgaattctaccagtgccata

SEQ ID NO: 80 - DNA, 124 bp, synthetic construct
  source 1..124: mol_type = other DNA; note = Exemplary mRNA destabilizing domain sequence
gagctcagtgtgagttctaccattgccaaactcgagcagtgaattctaccagtgccataggatccagtgtgagttctaccattgccaaaggtacccagtgaattctaccagtgccatagttaac

SEQ ID NO: 81 - DNA, 69 bp, synthetic construct
  source 1..69: mol_type = other DNA; note = Exemplary 3xFLAG tag sequence with stop codon
gactacaaagaccatgacggtgattataaagatcatgacatcgactacaaggatgacgatgacaagtaa

SEQ ID NO: 82 - DNA, 4786 bp, synthetic construct
  source 1..4786: mol_type = other DNA; note = Exemplary construct sequence
ctgcgcgctcgctcgctcactgaggccgcccgggcgtcgggcgacctttggtcgcccggcctcagtgagcgagcgagcgcgcagagagggagtggccaactccatcactaggggttcctgcggccgcacgcgtgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatgggtcgaggtgagccccacgttctgcttcactctccccatctcccccccctccccacccccaattttgtatttatttattttttaattattttgtgcagcgatgggggcggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgttgccttcgccccgtgccccgctccgcgccgcctcgcgccgcccgccccggctctgactgaccgcgttactcccacaggtgagcgggcgggacggcccttctcctccgggctgtaattagcgcttggtttaatgacggctcgtttcttttctgtggctgcgtgaaagccttaaagggctccgggagggccctttgtgcgggggggagcggctcggggggtgcgtgcgtgtgtgtgtgcgtggggagcgccgcgtgcggcccgcgctgcccggcggctgtgagcgctgcgggcgcggcgcggggctttgtgcgctccgcgtgtgcgcgaggggagcgcggccgggggcggtgccccgcggtgcgggggggctgcgaggggaacaaaggctgcgtgcggggtgtgtgcgtgggggggtgagcagggggtgtgggcgcggcggtcgggctgtaacccccccctgcacccccctccccgagttgctgagcacggcccggcttcgggtgcggggctccgtgcggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgaccggtgttgcggccccgcagcgcccgcgcgctcctctccccgactcggagcccctcggcggcgcccggcccaggacccgcctaggagcgcaggagccccagcgcagagaccccaacgccgagacccccgccccggccccgccgcgcttcctcccgacgcagtttaggacccttgttcgcgaagaggtggtgtgcggctgagacccgcgtcctcaggacggttccatcagtgcctcgatcctgccccactggaggaggaaggcagcccgaacagcgctcacctaactaacagctgctgagagctgggttccgtggccatgcacctgggactgccttgagaagcgtgagcaaaccgcccagagtagaagcgctagccaccatggattggggcacgctgcagacgatcctggggggtgtgaacaaacactccaccagcattggaaagatctggctcaccgtcctcttcatttttcgcattatgatcctcgttgtggctgcaaaggaggtgtggggagatgagcaggccgactttgtctgcaacaccctgcagccaggctgcaagaacgtgtgctacgatcactacttccccatctcccacatccggctatgggccctgcagctgatcttcgtgtccacgccagcgctcctagtggccatgcacgtggcctaccggagacatgagaagaagaggaagttcatcaagggggagataaagagtgaatttaaggacatcgaggagatcaaaacccagaaggtccgcatcgaaggctccctgtggtggacctacacaagcagcatcttcttccgggtcatcttcgaagccgccttcatgtacgtcttctatgtcatgtacgacggcttctccatgcagcggctggtgaagtgcaacgcctggccttgtcccaacactgtggactgctttgtgtcccggcccacggagaagactgtcttcacagtgttcatgattgcagtgtctggaatttgcatcctgctgaatgtcactgaattgtgttatttgctaattagatattgttctgggaagtcaaaaaagccagttggatcccgggctgactacaaagaccatgacggtgattataaagatcatgacatcgactacaaggatgacgatgacaagtaagaaatagacagcatgagagggatgaggcaacccgtgctcagctgtcaaggctcagtcgctagcatttcccaacacaaagattctgaccttaaatgcaaccatttgaaacccctgtaggcctcaggtgaaactccagatgccacaatggagctctgctcccctaaagcctcaaaacaaaggcctaattctatgcctgtcttaattttctttcacttaagttagttccactgagaccccaggctgttaggggttattggtgtaaggtactttcatattttaaacagaggatatcggcatttgtttctttctctgaggacaagagaaaaaagccaggttccacagaggacacagagaaggtttgggtgtcctcctggggttctttttgccaactttccccacgttaaaggtgaacattggttctttcatttgctttggaagttttaatctctaacagtggacaaagttaccagtgccttaaactctgttacactttttggaagtgaaaactttgtagtatgataggttattttgatgtaaagatgttctggataccattatatgttccccctgtttcagaggctcagattgtaatatgtaaatggtatgtcattcgctactatgatttaatttgaaatatggtcttttggttatgaatactttgcagcacagctgagaggctgtctgttgtattcattgtggtcatagcacctaacaacattgtagcctcaatcgagtgagacagactagaagttcctagtgatggcttatgatagcaaatggcctcatgtcaaatatttagatgtaattttgtgtaagaaatacagactggatgtaccaccaactactacctgtaatgacaggcctgtccaacacatctcccttttccatgactgtggtagccagcatcggaaagaacgctgatttaaagaggtcgcttgggaattttattgacacagtaccatttaatggggaggacaaaatggggcaggggagggagaagtttctgtcgttaaaaacagatttggaaagactggactctaaagtctgttgattaaagatgagctttgtctacttcaaaagtttgtttgcttaccccttcagcctccaattttttaagtgaaaatatagctaataacatgtgaaaagaatagaagctaaggtttagataaatattgagcagatctataggaagattgaacctgaatattgccattatgcttgacatggtttccaaaaaatggtactccacatatttcagtgagggtaagtattttcctgttgtcaagaatagcattgtaaaagcattttgtaataataaagaatagctttaatgatatgcttgtaactaaaataattttgtaatgtatcaaatacatttaaaacattaaaatataatctctataataagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggaagcttgaattcagctgacgtgcctcggaccgctaggaacccctagtgatggagttggccactccctctctgcgcgctcgctcgctcactgaggccgggcgaccaaaggtcgcccgacgcccgggctttgcccgggcggcctcagtgagcgagcgagcgcgcag

SEQ ID NO: 83 - DNA, 4018 bp, synthetic construct
  source 1..4018: mol_type = other DNA; note = Exemplary construct sequence
ctgcgcgctcgctcgctcactgaggccgcccgggcgtcgggcgacctttggtcgcccggcctcagtgagcgagcgagcgcgcagagagggagtggccaactccatcactaggggttcctgcggccgcacgcgtgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatgggtcgaggtgagccccacgttctgcttcactctccccatctcccccccctccccacccccaattttgtatttatttattttttaattattttgtgcagcgatgggggcggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgttgccttcgccccgtgccccgctccgcgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagctagagcctctgctaaccatgttcatgccttcttctttttcctacagctcctgggcaacgtgctggttattgtgaccggtgttgcggccccgcagcgcccgcgcgctcctctccccgactcggagcccctcggcggcgcccggcccaggacccgcctaggagcgcaggagccccagcgcagagaccccaacgccgagacccccgccccggccccgccgcgcttcctcccgacgcagtttaggacccttgttcgcgaagaggtggtgtgcggctgagacccgcgtcctcaggacggttccatcagtgcctcgatcctgccccactggaggaggaaggcagcccgaacagcgctcacctaactaacagctgctgagagctgggttccgtggccatgcacctgggactgccttgagaagcgtgagcaaaccgcccagagtagaagcgctagccaccatggattggggcacgctgcagacgatcctggggggtgtgaacaaacactccaccagcattggaaagatctggctcaccgtcctcttcatttttcgcattatgatcctcgttgtggctgcaaaggaggtgtggggagatgagcaggccgactttgtctgcaacaccctgcagccaggctgcaagaacgtgtgctacgatcactacttccccatctcccacatccggctatgggccctgcagctgatcttcgtgtccacgccagcgctcctagtggccatgcacgtggcctaccggagacatgagaagaagaggaagttcatcaagggggagataaagagtgaatttaaggacatcgaggagatcaaaacccagaaggtccgcatcgaaggctccctgtggtggacctacacaagcagcatcttcttccgggtcatcttcgaagccgccttcatgtacgtcttctatgtcatgtacgacggcttctccatgcagcggctggtgaagtgcaacgcctggccttgtcccaacactgtggactgctttgtgtcccggcccacggagaagactgtcttcacagtgttcatgattgcagtgtctggaatttgcatcctgctgaatgtcactgaattgtgttatttgctaattagatattgttctgggaagtcaaaaaagccagttggatcccgggctgactacaaagaccatgacggtgattataaagatcatgacatcgactacaaggatgacgatgacaagtaagaaatagacagcatgagagggatgaggcaacccgtgctcagctgtcaaggctcagtcgctagcatttcccaacacaaagattctgaccttaaatgcaaccatttgaaacccctgtaggcctcaggtgaaactccagatgccacaatggagctctgctcccctaaagcctcaaaacaaaggcctaattctatgcctgtcttaattttctttcacttaagttagttccactgagaccccaggctgttaggggttattggtgtaaggtactttcatattttaaacagaggatatcggcatttgtttctttctctgaggacaagagaaaaaagccaggttccacagaggacacagagaaggtttgggtgtcctcctggggttctttttgccaactttccccacgttaaaggtgaacattggttctttcatttgctttggaagttttaatctctaacagtggacaaagttaccagtgccttaaactctgttacactttttggaagtgaaaactttgtagtatgataggttattttgatgtaaagatgttctggataccattatatgttccccctgtttcagaggctcagattgtaatatgtaaatggtatgtcattcgctactatgatttaatttgaaatatggtcttttggttatgaatactttgcagcacagctgagaggctgtctgttgtattcattgtggtcatagcacctaacaacattgtagcctcaatcgagtgagacagactagaagttcctagtgatggcttatgatagcaaatggcctcatgtcaaatatttagatgtaattttgtgtaagaaatacagactggatgtaccaccaactactacctgtaatgacaggcctgtccaacacatctcccttttccatgactgtggtagccagcatcggaaagaacgctgatttaaagaggtcgcttgggaattttattgacacagtaccatttaatggggaggacaaaatggggcaggggagggagaagtttctgtcgttaaaaacagatttggaaagactggactctaaagtctgttgattaaagatgagctttgtctacttcaaaagtttgtttgcttaccccttcagcctccaattttttaagtgaaaatatagctaataacatgtgaaaagaatagaagctaaggtttagataaatattgagcagatctataggaagattgaacctgaatattgccattatgcttgacatggtttccaaaaaatggtactccacatatttcagtgagggtaagtattttcctgttgtcaagaatagcattgtaaaagcattttgtaataataaagaatagctttaatgatatgcttgtaactaaaataattttgtaatgtatcaaatacatttaaaacattaaaatataatctctataataagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggaagcttgaattcagctgacgtgcctcggaccgctaggaacccctagtgatggagttggccactccctctctgcgcgctcgctcgctcactgaggccgggcgaccaaaggtcgcccgacgcccgggctttgcccgggcggcctcagtgagcgagcgagcgcgcag

SEQ ID NO: 84 - DNA, 4508 bp, synthetic construct
  source 1..4508: mol_type = other DNA; note = Exemplary construct sequence
ctgcgcgctcgctcgctcactgaggccgcccgggcgtcgggcgacctttggtcgcccggcctcagtgagcgagcgagcgcgcagagagggagtggccaactccatcactaggggttcctgcggccgcacgcgtgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatgggtaagcttccgcagaatcctatcagtttccccctttcgtgctgtgtgcatcgagcaggaaggggcttggcaggttttacctgccctctttcctttctgaaaagtctgggcctcctcaccccgaaaggagtcacctccttgcagttccccagttgcgaaaagaggaggaagttggctgggccgggggccgcggggggcaccctccgcagatggcgggacccccctgccggccatggcaaaaacgaggcttgtctctcccaccgcccccaaccttagtccttggcacattgttgaaagtaattgaataaaatcggaaattcgagaaggcgttcgttcggattggtgagattttgaggggagaaagaagcggggacttcgccggcaccagcggcgccccctcctcggccaccgttaacccccattccagagggcactgccccgccacccagcctaggtccccctgcgagagcctcgcgggcccgcgcagcctccgcgactcgaacagatcttcagtccttggaggaatgcctgtttctctaacaataaaaaattaaagaagcgctcataaatgccaagtcctctcgcactatgcggagtacagaggacaacgaccacagccatccctgaaccccgcccacggcacagcgccggagccggggtctggggcgccgcttcctggggggtcccgactctcagccgcccccgcttcacccgggccgccaaggggctgggggaggcggcgctcggggtaaccgggggagactcagggcgctgggggcacttggggaactcatgggggctcaaaggaactaggagatcgggacctcgaaggggacttggggggttcggggctttcgggggcggtcgggggttcgcggacccgggaagctctgaggacccagaggccgggcgcgctccgcccgcggcgccgccccctccgtaactttcccagtctccgagggaagaggcggggtgtggggtgcggttaaaaggcgccacggcgggagacaggtctcctgggcaacgtgctggttattgtgaccggtgttgcggccccgcagcgcccgcgcgctcctctccccgactcggagcccctcggcggcgcccggcccaggacccgcctaggagcgcaggagccccagcgcagagaccccaacgccgagacccccgccccggccccgccgcgcttcctcccgacgcagtttaggacccttgttcgcgaagaggtggtgtgcggctgagacccgcgtcctcaggacggttccatcagtgcctcgatcctgccccactggaggaggaaggcagcccgaacagcgctcacctaactaacagctgctgagagctgggttccgtggccatgcacctgggactgccttgagaagcgtgagcaaaccgcccagagtagaagcgctagccaccatggattggggcacgctgcagacgatcctggggggtgtgaacaaacactccaccagcattggaaagatctggctcaccgtcctcttcatttttcgcattatgatcctcgttgtggctgcaaaggaggtgtggggagatgagcaggccgactttgtctgcaacaccctgcagccaggctgcaagaacgtgtgctacgatcactacttccccatctcccacatccggctatgggccctgcagctgatcttcgtgtccacgccagcgctcctagtggccatgcacgtggcctaccggagacatgagaagaagaggaagttcatcaagggggagataaagagtgaatttaaggacatcgaggagatcaaaacccagaaggtccgcatcgaaggctccctgtggtggacctacacaagcagcatcttcttccgggtcatcttcgaagccgccttcatgtacgtcttctatgtcatgtacgacggcttctccatgcagcggctggtgaagtgcaacgcctggccttgtcccaacactgtggactgctttgtgtcccggcccacggagaagactgtcttcacagtgttcatgattgcagtgtctggaatttgcatcctgctgaatgtcactgaattgtgttatttgctaattagatattgttctgggaagtcaaaaaagccagttggatcccgggctgactacaaagaccatgacggtgattataaagatcatgacatcgactacaaggatgacgatgacaagtaagaaatagacagcatgagagggatgaggcaacccgtgctcagctgtcaaggctcagtcgctagcatttcccaacacaaagattctgaccttaaatgcaaccatttgaaacccctgtaggcctcaggtgaaactccagatgccacaatggagctctgctcccctaaagcctcaaaacaaaggcctaattctatgcctgtcttaattttctttcacttaagttagttccactgagaccccaggctgttaggggttattggtgtaaggtactttcatattttaaacagaggatatcggcatttgtttctttctctgaggacaagagaaaaaagccaggttccacagaggacacagagaaggtttgggtgtcctcctggggttctttttgccaactttccccacgttaaaggtgaacattggttctttcatttgctttggaagttttaatctctaacagtggacaaagttaccagtgccttaaactctgttacactttttggaagtgaaaactttgtagtatgataggttattttgatgtaaagatgttctggataccattatatgttccccctgtttcagaggctcagattgtaatatgtaaatggtatgtcattcgctactatgatttaatttgaaatatggtcttttggttatgaatactttgcagcacagctgagaggctgtctgttgtattcattgtggtcatagcacctaacaacattgtagcctcaatcgagtgagacagactagaagttcctagtgatggcttatgatagcaaatggcctcatgtcaaatatttagatgtaattttgtgtaagaaatacagactggatgtaccaccaactactacctgtaatgacaggcctgtccaacacatctcccttttccatgactgtggtagccagcatcggaaagaacgctgatttaaagaggtcgcttgggaattttattgacacagtaccatttaatggggaggacaaaatggggcaggggagggagaagtttctgtcgttaaaaacagatttggaaagactggactctaaagtctgttgattaaagatgagctttgtctacttcaaaagtttgtttgcttaccccttcagcctccaattttttaagtgaaaatatagctaataacatgtgaaaagaatagaagctaaggtttagataaatattgagcagatctataggaagattgaacctgaatattgccattatgcttgacatggtttccaaaaaatggtactccacatatttcagtgagggtaagtattttcctgttgtcaagaatagcattgtaaaagcattttgtaataataaagaatagctttaatgatatgcttgtaactaaaataattttgtaatgtatcaaatacatttaaaacattaaaatataatctctataataagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggaagcttgaattcagctgacgtgcctcggaccgctaggaacccctagtgatggagttggccactccctctctgcgcgctcgctcgctcactgaggccgggcgaccaaaggtcgcccgacgcccgggctttgcccgggcggcctcagtgagcgagcgagcgcgcag

SEQ ID NO: 85 - skipped

SEQ ID NO: 86 - DNA, 128 bp, synthetic construct
  source 1..128: mol_type = other DNA; note = Exemplary hGJB2 basal promoter
aagctctgaggacccagaggccgggcgcgctccgcccgcggcgccgccccctccgtaactttcccagtctccgagggaagaggcggggtgtggggtgcggttaaaaggcgccacggcgggagacaggt

SEQ ID NO: 87 - DNA, 4745 bp, synthetic construct
  source 1..4745: mol_type = other DNA; note = Exemplary construct sequence
ctgcgcgctcgctcgctcactgaggccgcccgggcgtcgggcgacctttggtcgcccggcctcagtgagcgagcgagcgcgcagagagggagtggccaactccatcactaggggttcctgcggccgcacgcgtgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatgggtcgaggtgagccccacgttctgcttcactctccccatctcccccccctccccacccccaattttgtatttatttattttttaattattttgtgcagcgatgggggcggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgttgccttcgccccgtgccccgctccgcgccgcctcgcgccgcccgccccggctctgactgaccgcgttactcccacaggtgagcgggcgggacggcccttctcctccgggctgtaattagcgcttggtttaatgacggctcgtttcttttctgtggctgcgtgaaagccttaaagggctccgggagggccctttgtgcgggggggagcggctcggggggtgcgtgcgtgtgtgtgtgcgtggggagcgccgcgtgcggcccgcgctgcccggcggctgtgagcgctgcgggcgcggcgcggggctttgtgcgctccgcgtgtgcgcgaggggagcgcggccgggggcggtgccccgcggtgcgggggggctgcgaggggaacaaaggctgcgtgcggggtgtgtgcgtgggggggtgagcagggggtgtgggcgcggcggtcgggctgtaacccccccctgcacccccctccccgagttgctgagcacggcccggcttcgggtgcggggctccgtgcggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgaccggtgttgcggccccgcagcgcccgcgcgctcctctccccgactcggagcccctcggcggcgcccggcccaggacccgcctaggagcgcaggagccccagcgcagagaccccaacgccgagacccccgccccggccccgccgcgcttcctcccgacgcagtttaggacccttgttcgcgaagaggtggtgtgcggctgagacccgcgtcctcaggacggttccatcagtgcctcgatcctgccccactggaggaggaaggcagcccgaacagcgctcacctaactaacagctgctgagagctgggttccgtggccatgcacctgggactgccttgagaagcgtgagcaaaccgcccagagtagaagcgctagccaccatggattggggcacgctgcagacgatcctggggggtgtgaacaaacactccaccagcattggaaagatctggctcaccgtcctcttcatttttcgcattatgatcctcgttgtggctgcaaaggaggtgtggggagatgagcaggccgactttgtctgcaacaccctgcagccaggctgcaagaacgtgtgctacgatcactacttccccatctcccacatccggctatgggccctgcagctgatcttcgtgtccacgccagcgctcctagtggccatgcacgtggcctaccggagacatgagaagaagaggaagttcatcaagggggagataaagagtgaatttaaggacatcgaggagatcaaaacccagaaggtccgcatcgaaggctccctgtggtggacctacacaagcagcatcttcttccgggtcatcttcgaagccgccttcatgtacgtcttctatgtcatgtacgacggcttctccatgcagcggctggtgaagtgcaacgcctggccttgtcccaacactgtggactgctttgtgtcccggcccacggagaagactgtcttcacagtgttcatgattgcagtgtctggaatttgcatcctgctgaatgtcactgaattgtgttatttgctaattagatattgttctgggaagtcaaaaaagccagttggatcccgggctgactacaaagaccatgacggtgattataaagatcatgacatcgactacaaggatgacgatgacaagtaagagctcagtgtgagttctaccattgccaaactcgagcagtgaattctaccagtgccataggatccagtgtgagttctaccattgccaaaggtacccagtgaattctaccagtgccatagttaaccgcattgcccagttgttagattaagaaatagacagcatgagagggatgaggcaacccgtgctcagctgtcaaggctcagtcgctagcatttcccaacacaaagattctgaccttaaatgcaaccatttgaaacccctgtaggcctcaggtgaaactccagatgccacaatggagcctctgctcccctaaagcctcaaaacaaaggcctaattctatgcctgtcttaattttctttcacttaagttagttccactgagaccccaggctgttaggggttattggtgtaaggtactttcatattttaaacagaggatatcggcatttgtttctttctctgaggacaagagaaaaaagccaggttccacagaggacacagagaaggtttgggtgtcctcctggggttctttttgccaactttccccacgttaaaggtgaacattggttctttcatttgctttggaagttttaatctctaacagtggacaaagttaccagtgccttaaactctgttacactttttggaagtgaaaactttgtagtatgataggttattttgatgtaaagatgttctggataccattatatgttccccctgtttcagaggctcagattgtaatatgtaaatggtatgtcattcgctactatgatttaatttgaaatatggtcttttggttatgaatactttgcagcacagctgagaggctgtctgttgtattcattgtggtcatagcacctaacaacattgtagcctcaatcgagtgagacagactagaagttcctagtgatggcttatgatagcaaatggcctcatgtcaaatatttagatgtaattttgtgtaagaaatacagactggatgtaccaccaactactacctgtaatgacaggcctgtccaacacatctcccttttccatgactgtggtagccagcatcggaaagaacgctgatttaaagaggtcgcttgggaattttattgacacagtaccatttaatggggaggacaaaatggggcaggggagggagaagtttctgtcgttaaaaacagatttggaaagactggactctaaagtctgttgattaaagatgagctttgtctacttcaaaagtttgtttgcttaccccttcagcctccaattttttaagtgaaaatatagctaataacatgtgaaaagaatagaagctaaggtttagataaatattgagcagatctataggaagattgaacctgaatattgccattatgcttgacgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggaagcttgaattcagctgacgtgcctcggaccgctaggaacccctagtgatggagttggccactccctctctgcgcgctcgctcgctcactgaggccgggcgaccaaaggtcgcccgacgcccgggctttgcccgggcggcctcagtgagcgagcgagcgcgcag

SEQ ID NO: 88 - DNA, 4712 bp, synthetic construct
  source 1..4712: mol_type = other DNA; note = Exemplary construct sequence
ctgcgcgctcgctcgctcactgaggccgcccgggcgtcgggcgacctttggtcgcccggcctcagtgagcgagcgagcgcgcagagagggagtggccaactccatcactaggggttcctgcggccgcacgcgtcttcttctggagtcttttctggaataattctgggagtgggctcagcctgcgggagagtaacatttttataacttgatagatgtagctgagatgcctcccagaggggagacccgcctctcctccggcagctgtgcacgtaggcttgttcccagcagcctggccagggtggtccacctggtgtttctcatcttctttccccggagcgctgactcctgcgcgtcctcttggaagactcttgacaggacgggtgttttatgggtgtgattcagtgtcctcttgcatcagttcaatgtggtggtgttcaatcaacccttgtagcgttagcaaaatttgctcaagtcattccgcaggaatgtctgtgtcttgcttccaagaaagcttgtaagtgccggcaacaggccaagcagctcacaaacctgaccacaagcctgtgagtaattgtggggcagcacttagcagtcttttattttcgacttattaaagtctcatcttggcctcaccttctccctggaaggtggcgtgggtgggaaccactgggtcagatctttttcacccttgccgtggagccagtttcctgttgcatgtgggggaagcaacatgtggtgaagagtatagaaaacgaaaacatgtgggtacagtatgtataagtggagggaacaaactcataattccaactagtttctcatgagagactcatgaatcattgtggtagttctcaatataaacttaatctaggccggatgtggtggctcacacctgtaatctcagcactctgggtggatcacttgaggtcaggagtttgagaccagtctgaccaacatggagaaaccccatcgctactaaaaatacaaaattatccagatgtggtggctcacacctgtaatcccagcactttgggaggctgaggcgggtggatcacttgaggtcaggagtttgagaccagcctgaccaacatggagaaactgtgtctctactaaaaatacaaaattagctgggcgtggtgacgcatgcctgtaatcccagctatttggaggccgaagcaggaagcttccgcagaatcctatcagtttccccctttcgtgctgtgtgcatcgagcaggaaggggcttggcaggttttacctgccctctttcctttctgaaaagtctgggcctcctcaccccgaaaggagtcacctccttgcagttccccagttgcgaaaagaggaggaagttggctgggccgggggccgcggggggcaccctccgcagatggcgggacccccctgccggccatggcaaaaacgaggcttgtctctcccaccgcccccaaccttagtccttggcacattgttgaaagtaattgaataaaatcggaaattcgagaaggcgttcgttcggattggtgagattttgaggggagaaagaagcggggacttcgccggcaccagcggcgccccctcctcggccaccgttaacccccattccagagggcactgccccgccacccagcctaggtccccctgcgagagcctcgcgggcccgcgcagcctccgcgactcgaacagatcttcagtccttggaggaatgcctgtttctctaacaataaaaaattaaagaagcgctcataaatgccaagtcctctcgcactatgcggagtacagaggacaacgaccacagccatccctgaaccccgcccacggcacagcgccggagccggggtctggggcgccgcttcctggggggtcccgactctcagccgcccccgcttcacccgggccgccaaggggctgggggaggcggcgctcggggtaaccgggggagactcagggcgctgggggcacttggggaactcatgggggctcaaaggaactaggagatcgggacctcgaaggggacttggggggttcggggctttcgggggcggtcgggggttcgcggacccgggaagctctgaggacccagaggccgggcgcgctccgcccgcggcgccgccccctccgtaactttcccagtctccgagggaagaggcggggtgtggggtgcggttaaaaggcgccacggcgggagacaggtgttgcggccccgcagcgcccgcgcgctcctctccccgactcggagcccctcggcggcgcccggcccaggacccgcctaggagcgcaggagccccagcgcagagaccccaacgccgagacccccgccccggccccgccgcgcttcctcccgacgcagagcaaaccgcccagagtagaagaccggtcgctagccaccatggattggggcacgctgcagacgatcctggggggtgtgaacaaacactccaccagcattggaaagatctggctcaccgtcctcttcatttttcgcattatgatcctcgttgtggctgcaaaggaggtgtggggagatgagcaggccgactttgtctgcaacaccctgcagccaggctgcaagaacgtgtgctacgatcactacttccccatctcccacatccggctatgggccctgcagctgatcttcgtgtccacgccagcgctcctagtggccatgcacgtggcctaccggagacatgagaagaagaggaagttcatcaagggggagataaagagtgaatttaaggacatcgaggagatcaaaacccagaaggtccgcatcgaaggctccctgtggtggacctacacaagcagcatcttcttccgggtcatcttcgaagccgccttcatgtacgtcttctatgtcatgtacgacggcttctccatgcagcggctggtgaagtgcaacgcctggccttgtcccaacactgtggactgctttgtgtcccggcccacggagaagactgtcttcacagtgttcatgattgcagtgtctggaatttgcatcctgctgaatgtcactgaattgtgttatttgctaattagatattgttctgggaagtcaaaaaagccagttggatcccgggctgactacaaagaccatgacggtgattataaagatcatgacatcgactacaaggatgacgatgacaagtaagaaatagacagcatgagagggatgaggcaacccgtgctcagctgtcaaggctcagtcgctagcatttcccaacacaaagattctgaccttaaatgcaaccatttgaaacccctgtaggcctcaggtgaaactccagatgccacaatggagctctgctcccctaaagcctcaaaacaaaggcctaattctatgcctgtcttaattttctttcacttaagttagttccactgagaccccaggctgttaggggttattggtgtaaggtactttcatattttaaacagaggatatcggcatttgtttctttctctgaggacaagagaaaaaagccaggttccacagaggacacagagaaggtttgggtgtcctcctggggttctttttgccaactttccccacgttaaaggtgaacattggttctttcatttgctttggaagttttaatctctaacagtggacaaagttaccagtgccttaaactctgttacactttttggaagtgaaaactttgtagtatgataggttattttgatgtaaagatgttctggataccattatatgttccccctgtttcagaggctcagattgtaatatgtaaatggtatgtcattcgctactatgatttaatttgaaatatggtcttttggttatgaatactttgcagcacagctgagaggctgtctgttgtattcattgtggtcatagcacctaacaacattgtagcctcaatcgagtgagacagactagaagttcctagtgatggcttatgatagcaaatggcctcatgtcaaatatttagatgtaattttgtgtaagaaatacagactggatgtaccaccaactactacctgtaatgacaggcctgtccaacacatctcccttttccatgactgtggtagccagcatcggaaagaacgctgatttaaagaggtcgcttgggaattttattgacacagtaccatttaatggggaggacaaaatggggcaggggagggagaagtttctgtcgttaaaaacagatttggaaagactggactctaaagtctgttgattaaagatgagctttgtctacttcaaaagtttgtttgcttaccccttcagcctccaattttttaagtgaaaatatagctaataacatgtgaaaagaatagaagctaaggtttagataaatattgagcagatctataggaagattgaacctgaatattgccattatgcttgacatggtttccaaaaaatggtactccacatatttcagtgagggtaagtattttcctgttgtcaagaatagcattgtaaaagcattttgtaataataaagaatagctttaatgatatgcttgtaactaaaataattttgtaatgtatcaaatacatttaaaacattaaaatataatctctataataaaagcttgaattcagctgacgtgcctcggaccgctaggaacccctagtgatggagttggccactccctctctgcgcgctcgctcgctcactgaggccgggcgaccaaaggtcgcccgacgcccgggctttgcccgggcggcctcagtgagcgagcgagcgcgcag

SEQ ID NO: 89 - DNA, 17 bp, synthetic construct
  source 1..17: mol_type = other DNA; note = Exemplary sNRP poly(A) signal sequence
aaataaaatacgaaatg

SEQ ID NO: 90 - DNA, 1188 bp, synthetic construct
  source 1..1188: mol_type = other DNA; note = Exemplary GDF6 promoter
ccacaggtaactccgtcggcgtccacaggggggcaggagataccatactgcacagttgtacgtcttccatctgtttggtgtagaaaaatctaaccactacaagaatgccacgggcactgtggcagacagaagcagcgctacgccgcatcgcctttcagcgtgcaggcccaggaatgagcgaggcagtgggcggggaagacaggcacggggaatctggggacagataaaggaaactcgtgatggggcgaggctgggctgaagagaaacagattggggtagagctgcaaagggaggggtccactggaaggcgaggggggaggccgggaagagagagggtgggaaggcagtgtgagatgggagggcagtgtgagaagaaaagcaggctggggaagagggattggaatgcagaaggaacttggggaaggaggaagtcctgcaggcgggagggaaagaagagagggggagcagctaaagtctgcgtcagaagaggttggggactgcgagaggagaggctggggcctgcaggggagcgcagcagcttttagcatcgatccaaactctaaagactcgtggcctttgcctgacctcgagggtcgggaatagacgcctgtctttgtggagagcgatacccaaccgagaaaatggggctgttccgagctgggccctgcgcctggcccagggcgaggcttctctggctccgggctggcccctgaggggcagcacgcagcctgcagcagaggcgcctgctccaagctgtctcttgggggcgccgccgccgcttccctcctccggggccgctcgctcccaggaaagtggaggcggctggcgaggaccgagagccggggccgcgctgcggagggaccacacctccgggagttcgagggggaccctggcgcggcgggccagcctttcgggccggcagcgcccgccttcccccggtcagcgcttgcggcccgcgccgcgcgcaccgcccggcaaccccgcgcgcgtcccgcgggggcgctgcgtcttcctgccacaccggcgcaccgcggcccctctcccccacacctccggcccgcaccacccggctctcctcccaccctccccacccctcctctgccctccctccccattcctcccctcccggcgaggggcgggagggggcgtggcggggccggggtttgtgtggctgggacccggctcctc

SEQ ID NO: 91 - DNA, 680 bp, synthetic construct
  source 1..680: mol_type = other DNA; note = GFAP promoter
gaacatatcctggtgtggagtaggggacgctgctctgacagaggctcgggggcctgagctggctctgtgagctggggaggaggcagacagccaggccttgtctgcaagcagacctggcagcattgggctggccgccccccagggcctcctcttcatgcccagtgaatgactcaccttggcacagacacaatgttcggggtgggcacagtgcctgcttcccgccgcaccccagcccccctcaaatgccttccgagaagcccattgagcagggggcttgcattgcaccccagcctgacagcctggcatcttgggataaaagcagcacagccccctaggggctgcccttgctgtgtggcgccaccggcggtggagaacaaggctctattcagcctgtgcccaggaaaggggatcaggggatgcccaggcatggacagtgggtggcagggggggagaggagggctgtctgcttcccagaagtccaaggacacaaatgggtgaggggagctctccccatagctgggctgcggcccaaccccaccccctcaggctatgccagggggtgttgccaggggcacccgggcatcgccagtctagcccactccttcataaagccctcgcatcccaggagcgagcagagccagagcaggttggagaggagacgcatcacctccgctgctcgc

SEQ ID NO: 92 - DNA, 1404 bp, synthetic construct
  source 1..1404: mol_type = other DNA; note = BACE Promoter
tgtgctgcgagggcttcatctcctaagcactaaatgctaaattccccctcccacgcccatcgccactgtcctcacggatcctcgcagcagcttcccaatcggtctccctgtctccagcctcaccacccccaactaagaccattcatgaaaacagagacaaccaaggagacagtcacccaatgctgtcccttcagcttgcattattttctgacaagacagctctgccatccatggaagcctgtgtttgaagatctctgacataaaggtcccttgcagagctagacgtgattctaaaattgggaacacaggaataaaaatcaaatcttgagtagaagtagctgaaaattgcagtgattcggggaagcttggcttctaactccccactgtttgaagatgggcttgtttgttttttaaaacagccaacataattcagctggaggaggtacaaagaattttctattccttgtttctgtagaaatcgatggactttagcttgtctaattgtcccccctgcctttagtatctaaaataaaataaccctcgttgcttgcattactcaacgcatttctgcgtcttggcgtctatggctaaacgagtattaattagacagtccgcagagagctggctggggatagaaggggaggtgggggagaagggcagggatcacagcagggtggactcgtggccctgatttgggatcctgacagcaacttactaggtggcctgagggctgggtgccaggggaggcagcgggttccagtagcatctgacctgcattagggacaggggcgcggcggagggggcgaagggggcgggggtggggggaaggtggctggggtgaagcccagcttcgcagctagctgtgggcaacagagggagtaagggggggcaatgaggctggggccaggcgccagcagcagccacgccccccacctcccccgatttttagggaaaattctccaaagctctcgcatcctcctctgcctccttccaccctccaccctcccagcctccactgagacctctttaaaaccacccaggggccgccgggggatgaggccggggaacgggctggactgagggcgggggctcgggggcagcggacgggaaacgcctcgaaagcagccagacccggcgactgaaatgaggcggaggagcttggcgaggggaggcgcaggctcggaaaggcgcgcgaggctccaggctccttcccgatccaccgctctcctcgctgacctccgagtcacccccggaagctcccgccactgccgggcgaatagacccccgcggacccccaagcgcgcggggccggggccctagttcaggccctcgctgcccctttaagggttctcgaaactttccccccggtatcagatgagcctcgtcacatccgttggccgtggc

SEQ ID NO: 93 - DNA, 1467 bp, synthetic construct
  source 1..1467: mol_type = other DNA; note = DBI2 promoter
gaagaaacctgcatttcttacacttcagtgtactttccccatatttaactccaagatttttgttaatttgtttggttttcctttctcaaacaaaattatgctcagactgaaaaccctagatttgttccctattgcatcttcatttcttcccaaacattccataaaacgtgacctacattaagttagcaagttaagtctgaaagcgtctaccttccctggggagggggaaggtgtaggcagggcagagatttgtagtccagccctcttgccacaaattatgaattagagaggaatgactttgcttttttaatgatctccagagaattttccatcatttccctctcttcacccagctcctttgcaaccactgccagagaagtcttcctttagcttcttaaacatcgatcctaaaacacttccagacacctgtgctgctcctttcagttcccatggagattaggctgtgtaacaatctcgcaaagacgttcccctccgtctcctcatcctcttttcaaacccttttacgatttcccatctcactcagcatgacagtcaaagtccctgtgatggccaacttctgcatcacctagccagtctgccaccgccaaaactctccagcctcatcttacacttgttctctgcttggaatcttccctcccctccttgaggaactttctcaaatgtcaccttccctcaatactccccctcctccatttaaaactataaacttccaactctctaagcccctaaagtactctatatttaacttattgtataaactactgtccctacttgtaagttccaagattgcagggattcacccgctttgttcactgctgtctgccaaggtctagaacagtgcaagttacccaacaggagttcaataaacagccattcatttaacaaatatttgctgagcacttcgtcccgtccaagtttgttaaatcaagacaaataagacaccgtccctgcctttaacgcaccagatggagaaatgcaccacagacataaatgtgcaatacaggcctgacactacggccacaagcaagtcaaagaacgtgccaaaagttcagaggaagaagcctcggcttcgcctttcgggagaccagtccagctttccaccatcacgctgctcatcagggaccatctccgggggtctcctctagaccccaagggaggagcgggtcccgcccgccattcccaggtctcagagtttacttgtccagagatgcaacttccggcctcttcaggccgggcaagatttaaggaaagaaaagaaacataaggacctccgttcttcggtctccgtcccctccccttcccccgcgtgccccacctgttcccggcgtccccttcggctactcccggcgtttgcgcaagcggtcccacgtgggctcgggcggggctagcgccgcggcgggggctgggcacgcccctagcgcatagctggcttctgattggctttcc

SEQ ID NO: 94 - DNA, 1752 bp, synthetic construct
  source 1..1752: mol_type = other DNA; note = FABP3 promoter
taccattctgcctttcacctgatgttgctatcctcctccctcttgtttccttccacccatcctttccctcccacattactctcttatcccaccctattttacaaccagtagcctagggaaaagagcatagctcaaatgaggaagaaggcaggacaggcagtcatggcttagctggactgagctgcagtgcttctccttctggggaagggggtgcactgtcatctgctactgacacatccctccaaggcactcagccctgcagggagcaacctgattctatgactgacatctaatcttcacattcaccttgcaggaaggcaagaagtgatcccagcctccagatggaaagatcaaggcccagagaaggtcagtggtggttggaggcctgaggtcacacagcagccaagtctggagtcactagtcaaggtgaccttgactagccaccccacctccccttccctgccccaccatggccctgggagatctgttgtcctgtgagggaaaggggctccaggctgggctgcatctgaagcccctagatccagagacttcatttcttaggctatctataaaatccaccttcctttcttttcccaggacccccataccctgctcccagcatcgtctgcctcagctaagccatggggattgagagaccaggcctggtgcccagataaactgaccctgggtgaggggacaggggcccagaatgggcaggtagagactgaatactgaagaagaatcctctggagtctgttagcagaagcagatgggccttgcctgactattggcaggcggacctggtggtcagacctcagtgatcctcagggaccagtgaatatttcaggctggggctgagcatcacctgctcccttggccccacttatagggcaaaggggagtctaccagcctactcactgatgacaaactggaaaagtttgtcctgtctctgctctggccccacctcgccctctcccctacttggaagttcctttcctgaaccactgactgccaaagcttgagggattaaataaatcatctggcccaacctcctaccatagagttgggaacactgaagaaaagagactggcccaaggtcacagagaaggcagggtgaacactgtcacagggagagccagtgtagaataatggttaagccacgcaagctctagaaccactctatctgagtgcaaatcctggctgtcatctggtacttgcttcctggaacacatctggcctcagactcctgaggccaagacacactccctgccctaagacttgctggttctatggcaggcagaggcagaaagagccccaccatcattcccagcaaatgggaaaagttcccagttgcagatattaggggtgggatggggcgggggtagtcagcaaccatagacttagaccctgaagaggcaaaaaaggagggccatgttcttgggtcagcagagcttctactcagcttcttcagcctctagctctttcctggtgctagtagcacattctctagtggaggcatccagatggcagggagggtccaggaaacagctgaacatgctgagcaggcctcccttgtccccgctccccatggccccatggatcatccggtgctgcagctcatctcattggctggcttctggttactcatctctcctcttctccatcttcccagcctgtggttgccgtggaaacatagaacagtgacctcaccataggatgagggctggggagatgctgttcttggcaggcgct

SEQ ID NO: 95 - DNA, 1255 bp, synthetic construct
  source 1..1255: mol_type = other DNA; note = KLHL14 promoter
gaaacagcagccattgatgtagctcagggttctgtggatctgtcatttggagcatgttggttctcctgtctcagctgggctcattcatgcatctgagttcagctattgggcaatctggggaatgttttgtccatgtgatgtgtcatcttctaccaggctagcctgggcttcatcacatggtatctggcagggctctaagagggagagttgaaacacacaaggcctcttgaagcttagactcagaattggcacaaggtcgcttctggcacattccattggtcaaagcaagttacaaggccagctcacattcaaggattaggtaagtcgattccactcttgatgagaagtctgaaggatttggaacagtgtccaccatgcagtaataaactcaataagtagtagccattattattctgttagaggttgccaggaaaagttttatagtggaaagaaatctgagtttactcttgagaggtaagtggaatttctatttgtagagaatgaaggcctctcaaaaagacacagcctaacaataggtgctgcagtttaacagtggagcgtgtccagaacaggctgcccttttaggcaagggctagtgtctttcaggacagacccaaaccccaaataccaaaacagaataaagtagtgtcttagcatactttgagatcagactgtttctgcatttcacagtgctgggggtgggggggaggtgtggggggaagggaaaagcagcataccaatgtagtgaaatctggaaacaacagccaaaaaaagtttgcatattgcacagagcacttgaagatcataaatctatgcatgagaaagatgtagtggaaattttgggggggattagagtttatttttgtcatctctgtgagacagctactcattcatccagatcacagctaagaaaaaagctggtcacagaaattagcagtttcagctcagcagcgaagtcgccagcctgtgaaggcagagagaaattgactaattagcaatgcgcactaaaacttgacggttctttatagagagagagaagagagagggagagagagggagagggagggagggggggctcgctttttccccttctttcttccaaagatgtttgaaatcgcagtcatttacgctcgacaatttttacaatagccttgagccataattttgcgagtctctccagcatccatccccctgtatggtctctctctactggccaagcacgaccgtttctctccccaaccgtggatttcctatt

SEQ ID NO: 96 - DNA, 1012 bp, synthetic construct
  source 1..1012: mol_type = other DNA; note = MMP15 promoter
ccttcctcctccagggccctctgcagaccaggctgagatggaggaacctgctaaaatcgatggagatgcttctagcctcccagtaggaggccccagccatgccttcaacctggcaggaggtgtagccactcctcatccttgggttgcaggttgggtgctgctgttgtggtccttcccagaaactgccagtagagggcagcctgggcatcctaatgcttactctggttgttacacaaagaaaatattggggtcactggcgagcccacccacactcaccagaatctccactgtagtccccctaacaaacagcccttcacttcctctcccacttcagcaatttgtattttgatgccattggcctcagatcagagtgttttaaatcatcacgccctggcttatccctggtcgagccaggacacggggtgcttcagtgggtctgtcaccctctctccttgaagcatgttgcttttatttatttacttttactctcaccctgctcctgtaccagcaggggccacttcaaagccaaggtacagggtgataacttgtggtccagcatcagttttctccacttctttctcccactcacccccagcaaggtgcctggggagacttgagcagatgtttcattttggcctggccagtggctgaaagccaggcctccaatgcactgtgacctctggcttccccagcagctttcccagagaggcagagggagtcttcattcttcccaggcggggagaccacgccttccctgcctcctccctccgcggggggtcgcgttggaggtcacccccgccccctaggcgctgggttgggagtgacgcggggtgggctggagaggtttcctgccgtctgggaagcgtaaacggaccgcccacctgtcgggcctcggccgcccgcacctgcttgtgagaagcctgcggctggggcaccgcccccggtccccgcccgggtccgcgcattgggagcacactggccctttaagagcgcggcggccgcggcgcgcggg

SEQ ID NO: 97 - DNA, 1079 bp, synthetic construct
  source 1..1079: mol_type = other DNA; note = SPARC promoter
caggctacctctcaggctgactgagtcatgcagcataggctgccacgtctctgggctggcggggccgtcattattcctggcctcactgcagctaaattgaagaaacgtttggtttgtgggccacgtcaaggaatgtgtaagagctgccacgttgtcgggtctgggttattgggcttttcccctccttcagagaagatttccaggcgtgtgggtggggtttcagaagaaaattgatgcctgcgtgtgagtgttccctggacctggaccagcagcggcaatattacagacccgggggttggggcagactgagccaatctctgcaccgtcaaagttatggatacagagccctggaaaaaggctgaaggataagatagctgacatttatgaagtgcttcattcatgtagcagtgggccaaatgcgtactttacacttgaggaagctgaggctggaggttgataacatgcctcaagtcttctagagttaaataactttgacccaggacccaagcccagagttctgactcaaaaactaggcctcctaaacatcctcttatatgaggttaaatttcatcttcctctgtttggccttggcctggttggtggatgctctgcttcggggacccagggccagatgacaatgggttctttgtgcccttcagacaatgggaagggctgcctggggaaagatacagtaacaaggcaacaggctgagtcagcctccaatgtgcttgaaccttcttagcttggcagccttgacattcagccagccacacaaagggtatatcaaggatgataccactagtagcagcttgtcttgtctgtacctctgaacaagaaagaggctgttctgggtcatccctccaggcctgtccagccctggcactctgtgagtcggtttaggcagcagccccggaacagatgaggcaggcagggttgggacgtttggtcaggacagcccaccaggaggaaagaaatgaaagacagagacagctttggctatgggagaaggaggaggccgggggaaggaggagacaggaggaggagggaccacggggtggaggggagatagacccagccca

SEQ ID NO: 98 - DNA, 1223 bp, synthetic construct
  source 1..1223: mol_type = other DNA; note = TSPAN8 promoter
ccaaggactcttttttctaaacttcccttcatcttctagtttgacgcccttggtgggaaaagtgtctgagataaggaaaaggcatcctttcagttctctgatactatcttgaagcgagggatggagaaaggcaaagagagacacaggagaagcgtatcccctgggaacaggtgtctagtggagtccagtaactcacagtctctcagttccgtcagcactgtcccttgggtcgcaaatttcttccattagcccttccaccagctgtatttcaaatggggctggacaataattgtggccagtggccttgtgttgtttgtacttgcggactagtagttctcacctgtctttctctgactcctattagccactgggatttcagcagctggttcagccaattctactcaattcaacattaagttgcagtgggctagaactcatgggccgatttaacaagtgaaattctaccaagatacatcaaaaatagcaacaggactagatactcagctcattttgttttatttgtaatataccagttgtggctttagtgccagtctgattcatctctctactacaaaatgaggctctataaaggaaaatattgcaactggagtgaggaatttgaatcttataggaaggaatttgtcttctcatgaagacttcagtttaccagaagtatctattgaggaagtgtttacaagaaaatgtgccatttagctttattctaaatttgcataataactgaaccaaacaaaaaaatatagatagatagattgttctatctatagatagatagggaacattggcagtaggtggcagtaagttcccctgagcacatggaggacacagttaaatgcatttgaggtatgtgggaaatggtttaaagcagaattttatgccaacttttagtaacggaagcctaacaaatgtttgttctttcaagtgagagaagcaagcaatctggaactattcataagcttattttctgtatccttaaacatattttataatgaatgtatgatttaaatagtaagttaagtgtctgggggtactgcacacctcccttgcatacagtcaaacttcttcagggtgatggggaagaggagttataggctgccaagcaaaattgccaaactggtctcagaaattcactgcattggagagcgcgggatccttgcaacactgactttagcagttaaactagagtggttggggatgagtattct

SEQ ID NO: 99 - DNA, 1087 bp, synthetic construct
  source 1..1087: mol_type = other DNA; note = VIM promoter
attcacaatgcattccctctgcccaccacattaattatcaactccttttcctggcatttactcatccaacgcatggccccacgttaactttcagttccctttctcccctacaaatactccataatccagcaaccctgggatccctgagatgatgaagaggaccagtgcccattccaggagacatcaccgcagccctgaggaatcggctatgggcaccagcagggcacagtgccacacctcgccaatgccttgtcctccttttccatagtgagtcagtcagcaagcgtgtagaagtgagttccacactctcttcctcccatagggagatcacttttctcattctaagggttccaggcacactcacaatggtggcatttgctgagcagtggcttgaataaagggctctcagaaagcaagatgtaactcagagcataggctagccccaggaatgctcttggggaatgacctgcagcctcccagtgaaagagagaataaaagaaagccccagcaggcgagctgggcagtagagagtcctgtaattccaccttggcaagcaccatttgcaagaacgaactgggataaggtaaacaaaatattgcctaaaagaggcttgtccaaagaagtcagaatacgctcttcatttacctctaaatttcagtacaccataaatctaaatactcaaaaaaacctgtgccttttcaatcaaggtcaattgcacgaattcttttggaaaacaggacctatggcatttcccagacaaatcaccgtgaaccctgtactgtgcattgctgtcctaaaatccaaagattctgtcatttgtgttacataattgcctttcatttgaactcattaatcaaattggggtttttaagcaacacctaattaattctttaactggctcatccactgatcactgagttctattttgaaactacggacgtcgagtttcctctttcacccagaattttcagatcttgtttaaaaagttgggtgtggtttcatggggggagggggaagagcgagaggagaccagagggacgggggcggggactctgcaagaaaaaccttcccggtgcaatcgtgatct

SEQ ID NO: 100 - DNA, 4634 bp, synthetic construct
  source 1..4634: mol_type = other DNA; note = BACE2 construct
ctgcgcgctcgctcgctcactgaggccgcccgggcgtcgggcgacctttggtcgcccggcctcagtgagcgagcgagcgcgcagagagggagtggccaactccatcactaggggttcctgcggccgcacgcgtggttgtgctgcgagggcttcatctcctaagcactaaatgctaaattccccctcccacgcccatcgccactgtcctcacggatcctcgcagcagcttcccaatcggtctccctgtctccagcctcaccacccccaactaagaccattcatgaaaacagagacaaccaaggagacagtcacccaatgctgtcccttcagcttgcattattttctgacaagacagctctgccatccatggaagcctgtgtttgaagatctctgacataaaggtcccttgcagagctagacgtgattctaaaattgggaacacaggaataaaaatcaaatcttgagtagaagtagctgaaaattgcagtgattcggggaagcttggcttctaactccccactgtttgaagatgggcttgtttgttttttaaaacagccaacataattcagctggaggaggtacaaagaattttctattccttgtttctgtagaaatcgatggactttagcttgtctaattgtcccccctgcctttagtatctaaaataaaataaccctcgttgcttgcattactcaacgcatttctgcgtcttggcgtctatggctaaacgagtattaattagacagtccgcagagagctggctggggatagaaggggaggtgggggagaagggcagggatcacagcagggtggactcgtggccctgatttgggatcctgacagcaacttactaggtggcctgagggctgggtgccaggggaggcagcgggttccagtagcatctgacctgcattagggacaggggcgcggcggagggggcgaagggggcgggggtggggggaaggtggctggggtgaagcccagcttcgcagctagctgtgggcaacagagggagtaagggggggcaatgaggctggggccaggcgccagcagcagccacgccccccacctcccccgatttttagggaaaattctccaaagctctcgcatcctcctctgcctccttccaccctccaccctcccagcctccactgagacctctttaaaaccacccaggggccgccgggggatgaggccggggaacgggctggactgagggcgggggctcgggggcagcggacgggaaacgcctcgaaagcagccagacccggcgactgaaatgaggcggaggagcttggcgaggggaggcgcaggctcggaaaggcgcgcgaggctccaggctccttcccgatccaccgctctcctcgctgacctccgagtcacccccggaagctcccgccactgccgggcgaatagacccccgcggacccccaagcgcgcggggccggggccctagttcaggccctcgctgcccctttaagggttctcgaaactttccccccggtatcagatgagcctcgtcacatccgttggccgtggcaagctctgaggacccagaggccgggcgcgctccgcccgcggcgccgccccctccgtaactttcccagtctccgagggaagaggcggggtgtggggtgcggttaaaaggcgccacggcgggagacaggtctcaccggtcctacgctgttgcggccccgcagcgcccgcgcgctcctctccccgactcggagcccctcggcggcgcccggcccaggacccgcctaggagcgcaggagccccagcgcagagaccccaacgccgagacccccgccccggccccgccgcgcttcctcccgacgcagtttaggacccttgttcgcgaagaggtggtgtgcggctgagacccgcgtcctcaggacggttccatcagtgcctcgatcctgccccactggaggaggaaggcagcccgaacagcgctcacctaactaacagctgctgagagctgggttccgtggccatgcacctgggactgccttgagaagcgtgagcaaaccgcccagagtagaagcgctagccaccatggattggggcacgctgcagacgatcctggggggtgtgaacaaacactccaccagcattggaaagatctggctcaccgtcctcttcatttttcgcattatgatcctcgttgtggctgcaaaggaggtgtggggagatgagcaggccgactttgtctgcaacaccctgcagccaggctgcaagaacgtgtgctacgatcactacttccccatctcccacatccggctatgggccctgcagctgatcttcgtgtccacgccagcgctcctagtggccatgcacgtggcctaccggagacatgagaagaagaggaagttcatcaagggggagataaagagtgaatttaaggacatcgaggagatcaaaacccagaaggtccgcatcgaaggctccctgtggtggacctacacaagcagcatcttcttccgggtcatcttcgaagccgccttcatgtacgtcttctatgtcatgtacgacggcttctccatgcagcggctggtgaagtgcaacgcctggccttgtcccaacactgtggactgctttgtgtcccggcccacggagaagactgtcttcacagtgttcatgattgcagtgtctggaatttgcatcctgctgaatgtcactgaattgtgttatttgctaattagatattgttctgggaagtcaaaaaagccagttggatcccgggctgactacaaagaccatgacggtgattataaagatcatgacatcgactacaaggatgacgatgacaagtaagaaatagacagcatgagagggatgaggcaacccgtgctcagctgtcaaggctcagtcgctagcatttcccaacacaaagattctgaccttaaatgcaaccatttgaaacccctgtaggcctcaggtgaaactccagatgccacaatggagctctgctcccctaaagcctcaaaacaaaggcctaattctatgcctgtcttaattttctttcacttaagttagttccactgagaccccaggctgttaggggttattggtgtaaggtactttcatattttaaacagaggatatcggcatttgtttctttctctgaggacaagagaaaaaagccaggttccacagaggacacagagaaggtttgggtgtcctcctggggttctttttgccaactttccccacgttaaaggtgaacattggttctttcatttgctttggaagttttaatctctaacagtggacaaagttaccagtgccttaaactctgttacactttttggaagtgaaaactttgtagtatgataggttattttgatgtaaagatgttctggataccattatatgttccccctgtttcagaggctcagattgtaatatgtaaatggtatgtcattcgctactatgatttaatttgaaatatggtcttttggttatgaatactttgcagcacagctgagaggctgtctgttgtattcattgtggtcatagcacctaacaacattgtagcctcaatcgagtgagacagactagaagttcctagtgatggcttatgatagcaaatggcctcatgtcaaatatttagatgtaattttgtgtaagaaatacagactggatgtaccaccaactactacctgtaatgacaggcctgtccaacacatctcccttttccatgactgtggtagccagcatcggaaagaacgctgatttaaagaggtcgcttgggaattttattgacacagtaccatttaatggggaggacaaaatggggcaggggagggagaagtttctgtcgttaaaaacagatttggaaagactggactctaaagtctgttgattaaagatgagctttgtctacttcaaaagtttgtttgcttaccccttcagcctccaattttttaagtgaaaatatagctaataacatgtgaaaagaatagaagctaaggtttagataaatattgagcagatctataggaagattgaacctgaatattgccattatgcttgacatggtttccaaaaaatggtactccacatatttcagtgagggtaagtattttcctgttgtcaagaatagcattgtaaaagcattttgtaataataaagaatagctttaatgatatgcttgtaactaaaataattttgtaatgtatcaaatacatttaaaacattaaaatataatctctataataagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggaagcttgaattcagctgacgtgcctcggaccgctaggaacccctagtgatggagttggccactccctctctgcgcgctcgctcgctcactgaggccgggcgaccaaaggtcgcccgacgcccgggctttgcccgggcggcctcagtgagcgagcgagcgcgcag

SEQ ID NO: 101 - DNA, 4697 bp, synthetic construct
  source 1..4697: mol_type = other DNA; note = DBI2 construct
ctgcgcgctcgctcgctcactgaggccgcccgggcgtcgggcgacctttggtcgcccggcctcagtgagcgagcgagcgcgcagagagggagtggccaactccatcactaggggttcctgcggccgcacgcgtggtgaagaaacctgcatttcttacacttcagtgtactttccccatatttaactccaagatttttgttaatttgtttggttttcctttctcaaacaaaattatgctcagactgaaaaccctagatttgttccctattgcatcttcatttcttcccaaacattccataaaacgtgacctacattaagttagcaagttaagtctgaaagcgtctaccttccctggggagggggaaggtgtaggcagggcagagatttgtagtccagccctcttgccacaaattatgaattagagaggaatgactttgcttttttaatgatctccagagaattttccatcatttccctctcttcacccagctcctttgcaaccactgccagagaagtcttcctttagcttcttaaacatcgatcctaaaacacttccagacacctgtgctgctcctttcagttcccatggagattaggctgtgtaacaatctcgcaaagacgttcccctccgtctcctcatcctcttttcaaacccttttacgatttcccatctcactcagcatgacagtcaaagtccctgtgatggccaacttctgcatcacctagccagtctgccaccgccaaaactctccagcctcatcttacacttgttctctgcttggaatcttccctcccctccttgaggaactttctcaaatgtcaccttccctcaatactccccctcctccatttaaaactataaacttccaactctctaagcccctaaagtactctatatttaacttattgtataaactactgtccctacttgtaagttccaagattgcagggattcacccgctttgttcactgctgtctgccaaggtctagaacagtgcaagttacccaacaggagttcaataaacagccattcatttaacaaatatttgctgagcacttcgtcccgtccaagtttgttaaatcaagacaaataagacaccgtccctgcctttaacgcaccagatggagaaatgcaccacagacataaatgtgcaatacaggcctgacactacggccacaagcaagtcaaagaacgtgccaaaagttcagaggaagaagcctcggcttcgcctttcgggagaccagtccagctttccaccatcacgctgctcatcagggaccatctccgggggtctcctctagaccccaagggaggagcgggtcccgcccgccattcccaggtctcagagtttacttgtccagagatgcaacttccggcctcttcaggccgggcaagatttaaggaaagaaaagaaacataaggacctccgttcttcggtctccgtcccctccccttcccccgcgtgccccacctgttcccggcgtccccttcggctactcccggcgtttgcgcaagcggtcccacgtgggctcgggcggggctagcgccgcggcgggggctgggcacgcccctagcgcatagctggcttctgattggctttccaagctctgaggacccagaggccgggcgcgctccgcccgcggcgccgccccctccgtaactttcccagtctccgagggaagaggcggggtgtggggtgcggttaaaaggcgccacggcgggagacaggtctcaccggtgccaaagcgttgcggccccgcagcgcccgcgcgctcctctccccgactcggagcccctcggcggcgcccggcccaggacccgcctaggagcgcaggagccccagcgcagagaccccaacgccgagacccccgccccggccccgccgcgcttcctcccgacgcagtttaggacccttgttcgcgaagaggtggtgtgcggctgagacccgcgtcctcaggacggttccatcagtgcctcgatcctgccccactggaggaggaaggcagcccgaacagcgctcacctaactaacagctgctgagagctgggttccgtggccatgcacctgggactgccttgagaagcgtgagcaaaccgcccagagtagaagcgctagccaccatggattggggcacgctgcagacgatcctggggggtgtgaacaaacactccaccagcattggaaagatctggctcaccgtcctcttcatttttcgcattatgatcctcgttgtggctgcaaaggaggtgtggggagatgagcaggccgactttgtctgcaacaccctgcagccaggctgcaagaacgtgtgctacgatcactacttccccatctcccacatccggctatgggccctgcagctgatcttcgtgtccacgccagcgctcctagtggccatgcacgtggcctaccggagacatgagaagaagaggaagttcatcaagggggagataaagagtgaatttaaggacatcgaggagatcaaaacccagaaggtccgcatcgaaggctccctgtggtggacctacacaagcagcatcttcttccgggtcatcttcgaagccgccttcatgtacgtcttctatgtcatgtacgacggcttctccatgcagcggctggtgaagtgcaacgcctggccttgtcccaacactgtggactgctttgtgtcccggcccacggagaagactgtcttcacagtgttcatgattgcagtgtctggaatttgcatcctgctgaatgtcactgaattgtgttatttgctaattagatattgttctgggaagtcaaaaaagccagttggatcccgggctgactacaaagaccatgacggtgattataaagatcatgacatcgactacaaggatgacgatgacaagtaagaaatagacagcatgagagggatgaggcaacccgtgctcagctgtcaaggctcagtcgctagcatttcccaacacaaagattctgaccttaaatgcaaccatttgaaacccctgtaggcctcaggtgaaactccagatgccacaatggagctctgctcccctaaagcctcaaaacaaaggcctaattctatgcctgtcttaattttctttcacttaagttagttccactgagaccccaggctgttaggggttattggtgtaaggtactttcatattttaaacagaggatatcggcatttgtttctttctctgaggacaagagaaaaaagccaggttccacagaggacacagagaaggtttgggtgtcctcctggggttctttttgccaactttccccacgttaaaggtgaacattggttctttcatttgctttggaagttttaatctctaacagtggacaaagttaccagtgccttaaactctgttacactttttggaagtgaaaactttgtagtatgataggttattttgatgtaaagatgttctggataccattatatgttccccctgtttcagaggctcagattgtaatatgtaaatggtatgtcattcgctactatgatttaatttgaaatatggtcttttggttatgaatactttgcagcacagctgagaggctgtctgttgtattcattgtggtcatagcacctaacaacattgtagcctcaatcgagtgagacagactagaagttcctagtgatggcttatgatagcaaatggcctcatgtcaaatatttagatgtaattttgtgtaagaaatacagactggatgtaccaccaactactacctgtaatgacaggcctgtccaacacatctcccttttccatgactgtggtagccagcatcggaaagaacgctgatttaaagaggtcgcttgggaattttattgacacagtaccatttaatggggaggacaaaatggggcaggggagggagaagtttctgtcgttaaaaacagatttggaaagactggactctaaagtctgttgattaaagatgagctttgtctacttcaaaagtttgtttgcttaccccttcagcctccaattttttaagtgaaaatatagctaataacatgtgaaaagaatagaagctaaggtttagataaatattgagcagatctataggaagattgaacctgaatattgccattatgcttgacatggtttccaaaaaatggtactccacatatttcagtgagggtaagtattttcctgttgtcaagaatagcattgtaaaagcattttgtaataataaagaatagctttaatgatatgcttgtaactaaaataattttgtaatgtatcaaatacatttaaaacattaaaatataatctctataataagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggaagcttgaattcagctgacgtgcctcggaccgctaggaacccctagtgatggagttggccactccctctctgcgcgctcgctcgctcactgaggccgggcgaccaaaggtcgcccgacgcccgggctttgcccgggcggcctcagtgagcgagcgagcgcgcag

SEQ ID NO: 102 - DNA, 4982 bp, synthetic construct
  source 1..4982: mol_type = other DNA; note = FABP3 construct
ctgcgcgctcgctcgctcactgaggccgcccgggcgtcgggcgacctttggtcgcccggcctcagtgagcgagcgagcgcgcagagagggagtggccaactccatcactaggggttcctgcggccgcacgcgtggttaccattctgcctttcacctgatgttgctatcctcctccctcttgtttccttccacccatcctttccctcccacattactctcttatcccaccctattttacaaccagtagcctagggaaaagagcatagctcaaatgaggaagaaggcaggacaggcagtcatggcttagctggactgagctgcagtgcttctccttctggggaagggggtgcactgtcatctgctactgacacatccctccaaggcactcagccctgcagggagcaacctgattctatgactgacatctaatcttcacattcaccttgcaggaaggcaagaagtgatcccagcctccagatggaaagatcaaggcccagagaaggtcagtggtggttggaggcctgaggtcacacagcagccaagtctggagtcactagtcaaggtgaccttgactagccaccccacctccccttccctgccccaccatggccctgggagatctgttgtcctgtgagggaaaggggctccaggctgggctgcatctgaagcccctagatccagagacttcatttcttaggctatctataaaatccaccttcctttcttttcccaggacccccataccctgctcccagcatcgtctgcctcagctaagccatggggattgagagaccaggcctggtgcccagataaactgaccctgggtgaggggacaggggcccagaatgggcaggtagagactgaatactgaagaagaatcctctggagtctgttagcagaagcagatgggccttgcctgactattggcaggcggacctggtggtcagacctcagtgatcctcagggaccagtgaatatttcaggctggggctgagcatcacctgctcccttggccccacttatagggcaaaggggagtctaccagcctactcactgatgacaaactggaaaagtttgtcctgtctctgctctggccccacctcgccctctcccctacttggaagttcctttcctgaaccactgactgccaaagcttgagggattaaataaatcatctggcccaacctcctaccatagagttgggaacactgaagaaaagagactggcccaaggtcacagagaaggcagggtgaacactgtcacagggagagccagtgtagaataatggttaagccacgcaagctctagaaccactctatctgagtgcaaatcctggctgtcatctggtacttgcttcctggaacacatctggcctcagactcctgaggccaagacacactccctgccctaagacttgctggttctatggcaggcagaggcagaaagagccccaccatcattcccagcaaatgggaaaagttcccagttgcagatattaggggtgggatggggcgggggtagtcagcaaccatagacttagaccctgaagaggcaaaaaaggagggccatgttcttgggtcagcagagcttctactcagcttcttcagcctctagctctttcctggtgctagtagcacattctctagtggaggcatccagatggcagggagggtccaggaaacagctgaacatgctgagcaggcctcccttgtccccgctccccatggccccatggatcatccggtgctgcagctcatctcattggctggcttctggttactcatctctcctcttctccatcttcccagcctgtggttgccgtggaaacatagaacagtgacctcaccataggatgagggctggggagatgctgttcttggcaggcgctaagctctgaggacccagaggccgggcgcgctccgcccgcggcgccgccccctccgtaactttcccagtctccgagggaagaggcggggtgtggggtgcggttaaaaggcgccacggcgggagacaggtctcaccggtccatccacgttgcggccccgcagcgcccgcgcgctcctctccccgactcggagcccctcggcggcgcccggcccaggacccgcctaggagcgcaggagccccagcgcagagaccccaacgccgagacccccgccccggccccgccgcgcttcctcccgacgcagtttaggacccttgttcgcgaagaggtggtgtgcggctgagacccgcgtcctcaggacggttccatcagtgcctcgatcctgccccactggaggaggaaggcagcccgaacagcgctcacctaactaacagctgctgagagctgggttccgtggccatgcacctgggactgccttgagaagcgtgagcaaaccgcccagagtagaagcgctagccaccatggattggggcacgctgcagacgatcctggggggtgtgaacaaacactccaccagcattggaaagatctggctcaccgtcctcttcatttttcgcattatgatcctcgttgtggctgcaaaggaggtgtggggagatgagcaggccgactttgtctgcaacaccctgcagccaggctgcaagaacgtgtgctacgatcactacttccccatctcccacatccggctatgggccctgcagctgatcttcgtgtccacgccagcgctcctagtggccatgcacgtggcctaccggagacatgagaagaagaggaagttcatcaagggggagataaagagtgaatttaaggacatcgaggagatcaaaacccagaaggtccgcatcgaaggctccctgtggtggacctacacaagcagcatcttcttccgggtcatcttcgaagccgccttcatgtacgtcttctatgtcatgtacgacggcttctccatgcagcggctggtgaagtgcaacgcctggccttgtcccaacactgtggactgctttgtgtcccggcccacggagaagactgtcttcacagtgttcatgattgcagtgtctggaatttgcatcctgctgaatgtcactgaattgtgttatttgctaattagatattgttctgggaagtcaaaaaagccagttggatcccgggctgactacaaagaccatgacggtgattataaagatcatgacatcgactacaaggatgacgatgacaagtaagaaatagacagcatgagagggatgaggcaacccgtgctcagctgtcaaggctcagtcgctagcatttcccaacacaaagattctgaccttaaatgcaaccatttgaaacccctgtaggcctcaggtgaaactccagatgccacaatggagctctgctcccctaaagcctcaaaacaaaggcctaattctatgcctgtcttaattttctttcacttaagttagttccactgagaccccaggctgttaggggttattggtgtaaggtactttcatattttaaacagaggatatcggcatttgtttctttctctgaggacaagagaaaaaagccaggttccacagaggacacagagaaggtttgggtgtcctcctggggttctttttgccaactttccccacgttaaaggtgaacattggttctttcatttgctttggaagttttaatctctaacagtggacaaagttaccagtgccttaaactctgttacactttttggaagtgaaaactttgtagtatgataggttattttgatgtaaagatgttctggataccattatatgttccccctgtttcagaggctcagattgtaatatgtaaatggtatgtcattcgctactatgatttaatttgaaatatggtcttttggttatgaatactttgcagcacagctgagaggctgtctgttgtattcattgtggtcatagcacctaacaacattgtagcctcaatcgagtgagacagactagaagttcctagtgatggcttatgatagcaaatggcctcatgtcaaatatttagatgtaattttgtgtaagaaatacagactggatgtaccaccaactactacctgtaatgacaggcctgtccaacacatctcccttttccatgactgtggtagccagcatcggaaagaacgctgatttaaagaggtcgcttgggaattttattgacacagtaccatttaatggggaggacaaaatggggcaggggagggagaagtttctgtcgttaaaaacagatttggaaagactggactctaaagtctgttgattaaagatgagctttgtctacttcaaaagtttgtttgcttaccccttcagcctccaattttttaagtgaaaatatagctaataacatgtgaaaagaatagaagctaaggtttagataaatattgagcagatctataggaagattgaacctgaatattgccattatgcttgacatggtttccaaaaaatggtactccacatatttcagtgagggtaagtattttcctgttgtcaagaatagcattgtaaaagcattttgtaataataaagaatagctttaatgatatgcttgtaactaaaataattttgtaatgtatcaaatacatttaaaacattaaaatataatctctataataagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggaagcttgaattcagctgacgtgcctcggaccgctaggaacccctagtgatggagttggccactccctctctgcgcgctcgctcgctcactgaggccgggcgaccaaaggtcgcccgacgcccgggctttgcccgggcggcctcagtgagcgagcgagcgcgcag

SEQ ID NO: 103 - DNA, 4485 bp, synthetic construct
  source 1..4485: mol_type = other DNA; note = KLHL14 construct
ctgcgcgctcgctcgctcactgaggccgcccgggcgtcgggcgacctttggtcgcccggcctcagtgagcgagcgagcgcgcagagagggagtggccaactccatcactaggggttcctgcggccgcacgcgtggtgaaacagcagccattgatgtagctcagggttctgtggatctgtcatttggagcatgttggttctcctgtctcagctgggctcattcatgcatctgagttcagctattgggcaatctggggaatgttttgtccatgtgatgtgtcatcttctaccaggctagcctgggcttcatcacatggtatctggcagggctctaagagggagagttgaaacacacaaggcctcttgaagcttagactcagaattggcacaaggtcgcttctggcacattccattggtcaaagcaagttacaaggccagctcacattcaaggattaggtaagtcgattccactcttgatgagaagtctgaaggatttggaacagtgtccaccatgcagtaataaactcaataagtagtagccattattattctgttagaggttgccaggaaaagttttatagtggaaagaaatctgagtttactcttgagaggtaagtggaatttctatttgtagagaatgaaggcctctcaaaaagacacagcctaacaataggtgctgcagtttaacagtggagcgtgtccagaacaggctgcccttttaggcaagggctagtgtctttcaggacagacccaaaccccaaataccaaaacagaataaagtagtgtcttagcatactttgagatcagactgtttctgcatttcacagtgctgggggtgggggggaggtgtggggggaagggaaaagcagcataccaatgtagtgaaatctggaaacaacagccaaaaaaagtttgcatattgcacagagcacttgaagatcataaatctatgcatgagaaagatgtagtggaaattttgggggggattagagtttatttttgtcatctctgtgagacagctactcattcatccagatcacagctaagaaaaaagctggtcacagaaattagcagtttcagctcagcagcgaagtcgccagcctgtgaaggcagagagaaattgactaattagcaatgcgcactaaaacttgacggttctttatagagagagagaagagagagggagagagagggagagggagggagggggggctcgctttttccccttctttcttccaaagatgtttgaaatcgcagtcatttacgctcgacaatttttacaatagccttgagccataattttgcgagtctctccagcatccatccccctgtatggtctctctctactggccaagcacgaccgtttctctccccaaccgtggatttcctattaagctctgaggacccagaggccgggcgcgctccgcccgcggcgccgccccctccgtaactttcccagtctccgagggaagaggcggggtgtggggtgcggttaaaaggcgccacggcgggagacaggtctcaccggtcccgttctgttgcggccccgcagcgcccgcgcgctcctctccccgactcggagcccctcggcggcgcccggcccaggacccgcctaggagcgcaggagccccagcgcagagaccccaacgccgagacccccgccccggccccgccgcgcttcctcccgacgcagtttaggacccttgttcgcgaagaggtggtgtgcggctgagacccgcgtcctcaggacggttccatcagtgcctcgatcctgccccactggaggaggaaggcagcccgaacagcgctcacctaactaacagctgctgagagctgggttccgtggccatgcacctgggactgccttgagaagcgtgagcaaaccgcccagagtagaagcgctagccaccatggattggggcacgctgcagacgatcctggggggtgtgaacaaacactccaccagcattggaaagatctggctcaccgtcctcttcatttttcgcattatgatcctcgttgtggctgcaaaggaggtgtggggagatgagcaggccgactttgtctgcaacaccctgcagccaggctgcaagaacgtgtgctacgatcactacttccccatctcccacatccggctatgggccctgcagctgatcttcgtgtccacgccagcgctcctagtggccatgcacgtggcctaccggagacatgagaagaagaggaagttcatcaagggggagataaagagtgaatttaaggacatcgaggagatcaaaacccagaaggtccgcatcgaaggctccctgtggtggacctacacaagcagcatcttcttccgggtcatcttcgaagccgccttcatgtacgtcttctatgtcatgtacgacggcttctccatgcagcggctggtgaagtgcaacgcctggccttgtcccaacactgtggactgctttgtgtcccggcccacggagaagactgtcttcacagtgttcatgattgcagtgtctggaatttgcatcctgctgaatgtcactgaattgtgttatttgctaattagatattgttctgggaagtcaaaaaagccagttggatcccgggctgactacaaagaccatgacggtgattataaagatcatgacatcgactacaaggatgacgatgacaagtaagaaatagacagcatgagagggatgaggcaacccgtgctcagctgtcaaggctcagtcgctagcatttcccaacacaaagattctgaccttaaatgcaaccatttgaaacccctgtaggcctcaggtgaaactccagatgccacaatggagctctgctcccctaaagcctcaaaacaaaggcctaattctatgcctgtcttaattttctttcacttaagttagttccactgagaccccaggctgttaggggttattggtgtaaggtactttcatattttaaacagaggatatcggcatttgtttctttctctgaggacaagagaaaaaagccaggttccacagaggacacagagaaggtttgggtgtcctcctggggttctttttgccaactttccccacgttaaaggtgaacattggttctttcatttgctttggaagttttaatctctaacagtggacaaagttaccagtgccttaaactctgttacactttttggaagtgaaaactttgtagtatgataggttattttgatgtaaagatgttctggataccattatatgttccccctgtttcagaggctcagattgtaatatgtaaatggtatgtcattcgctactatgatttaatttgaaatatggtcttttggttatgaatactttgcagcacagctgagaggctgtctgttgtattcattgtggtcatagcacctaacaacattgtagcctcaatcgagtgagacagactagaagttcctagtgatggcttatgatagcaaatggcctcatgtcaaatatttagatgtaattttgtgtaagaaatacagactggatgtaccaccaactactacctgtaatgacaggcctgtccaacacatctcccttttccatgactgtggtagccagcatcggaaagaacgctgatttaaagaggtcgcttgggaattttattgacacagtaccatttaatggggaggacaaaatggggcaggggagggagaagtttctgtcgttaaaaacagatttggaaagactggactctaaagtctgttgattaaagatgagctttgtctacttcaaaagtttgtttgcttaccccttcagcctccaattttttaagtgaaaatatagctaataacatgtgaaaagaatagaagctaaggtttagataaatattgagcagatctataggaagattgaacctgaatattgccattatgcttgacatggtttccaaaaaatggtactccacatatttcagtgagggtaagtattttcctgttgtcaagaatagcattgtaaaagcattttgtaataataaagaatagctttaatgatatgcttgtaactaaaataattttgtaatgtatcaaatacatttaaaacattaaaatataatctctataataagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggaagcttgaattcagctgacgtgcctcggaccgctaggaacccctagtgatggagttggccactccctctctgcgcgctcgctcgctcactgaggccgggcgaccaaaggtcgcccgacgcccgggctttgcccgggcggcctcagtgagcgagcgagcgcgcag

SEQ ID NO: 104 - DNA, 4242 bp, synthetic construct
  source 1..4242: mol_type = other DNA; note = MMP15 construct
ctgcgcgctcgctcgctcactgaggccgcccgggcgtcgggcgacctttggtcgcccggcctcagtgagcgagcgagcgcgcagagagggagtggccaactccatcactaggggttcctgcggccgcacgcgtggtccttcctcctccagggccctctgcagaccaggctgagatggaggaacctgctaaaatcgatggagatgcttctagcctcccagtaggaggccccagccatgccttcaacctggcaggaggtgtagccactcctcatccttgggttgcaggttgggtgctgctgttgtggtccttcccagaaactgccagtagagggcagcctgggcatcctaatgcttactctggttgttacacaaagaaaatattggggtcactggcgagcccacccacactcaccagaatctccactgtagtccccctaacaaacagcccttcacttcctctcccacttcagcaatttgtattttgatgccattggcctcagatcagagtgttttaaatcatcacgccctggcttatccctggtcgagccaggacacggggtgcttcagtgggtctgtcaccctctctccttgaagcatgttgcttttatttatttacttttactctcaccctgctcctgtaccagcaggggccacttcaaagccaaggtacagggtgataacttgtggtccagcatcagttttctccacttctttctcccactcacccccagcaaggtgcctggggagacttgagcagatgtttcattttggcctggccagtggctgaaagccaggcctccaatgcactgtgacctctggcttccccagcagctttcccagagaggcagagggagtcttcattcttcccaggcggggagaccacgccttccctgcctcctccctccgcggggggtcgcgttggaggtcacccccgccccctaggcgctgggttgggagtgacgcggggtgggctggagaggtttcctgccgtctgggaagcgtaaacggaccgcccacctgtcgggcctcggccgcccgcacctgcttgtgagaagcctgcggctggggcaccgcccccggtccccgcccgggtccgcgcattgggagcacactggccctttaagagcgcggcggccgcggcgcgcgggaagctctgaggacccagaggccgggcgcgctccgcccgcggcgccgccccctccgtaactttcccagtctccgagggaagaggcggggtgtggggtgcggttaaaaggcgccacggcgggagacaggtctcaccggtttcactgggttgcggccccgcagcgcccgcgcgctcctctccccgactcggagcccctcggcggcgcccggcccaggacccgcctaggagcgcaggagccccagcgcagagaccccaacgccgagacccccgccccggccccgccgcgcttcctcccgacgcagtttaggacccttgttcgcgaagaggtggtgtgcggctgagacccgcgtcctcaggacggttccatcagtgcctcgatcctgccccactggaggaggaaggcagcccgaacagcgctcacctaactaacagctgctgagagctgggttccgtggccatgcacctgggactgccttgagaagcgtgagcaaaccgcccagagtagaagcgctagccaccatggattggggcacgctgcagacgatcctggggggtgtgaacaaacactccaccagcattggaaagatctggctcaccgtcctcttcatttttcgcattatgatcctcgttgtggctgcaaaggaggtgtggggagatgagcaggccgactttgtctgcaacaccctgcagccaggctgcaagaacgtgtgctacgatcactacttccccatctcccacatccggctatgggccctgcagctgatcttcgtgtccacgccagcgctcctagtggccatgcacgtggcctaccggagacatgagaagaagaggaagttcatcaagggggagataaagagtgaatttaaggacatcgaggagatcaaaacccagaaggtccgcatcgaaggctccctgtggtggacctacacaagcagcatcttcttccgggtcatcttcgaagccgccttcatgtacgtcttctatgtcatgtacgacggcttctccatgcagcggctggtgaagtgcaacgcctggccttgtcccaacactgtggactgctttgtgtcccggcccacggagaagactgtcttcacagtgttcatgattgcagtgtctggaatttgcatcctgctgaatgtcactgaattgtgttatttgctaattagatattgttctgggaagtcaaaaaagccagttggatcccgggctgactacaaagaccatgacggtgattataaagatcatgacatcgactacaaggatgacgatgacaagtaagaaatagacagcatgagagggatgaggcaacccgtgctcagctgtcaaggctcagtcgctagcatttcccaacacaaagattctgaccttaaatgcaaccatttgaaacccctgtaggcctcaggtgaaactccagatgccacaatggagctctgctcccctaaagcctcaaaacaaaggcctaattctatgcctgtcttaattttctttcacttaagttagttccactgagaccccaggctgttaggggttattggtgtaaggtactttcatattttaaacagaggatatcggcatttgtttctttctctgaggacaagagaaaaaagccaggttccacagaggacacagagaaggtttgggtgtcctcctggggttctttttgccaactttccccacgttaaaggtgaacattggttctttcatttgctttggaagttttaatctctaacagtggacaaagttaccagtgccttaaactctgttacactttttggaagtgaaaactttgtagtatgataggttattttgatgtaaagatgttctggataccattatatgttccccctgtttcagaggctcagattgtaatatgtaaatggtatgtcattcgctactatgatttaatttgaaatatggtcttttggttatgaatactttgcagcacagctgagaggctgtctgttgtattcattgtggtcatagcacctaacaacattgtagcctcaatcgagtgagacagactagaagttcctagtgatggcttatgatagcaaatggcctcatgtcaaatatttagatgtaattttgtgtaagaaatacagactggatgtaccaccaactactacctgtaatgacaggcctgtccaacacatctcccttttccatgactgtggtagccagcatcggaaagaacgctgatttaaagaggtcgcttgggaattttattgacacagtaccatttaatggggaggacaaaatggggcaggggagggagaagtttctgtcgttaaaaacagatttggaaagactggactctaaagtctgttgattaaagatgagctttgtctacttcaaaagtttgtttgcttaccccttcagcctccaattttttaagtgaaaatatagctaataacatgtgaaaagaatagaagctaaggtttagataaatattgagcagatctataggaagattgaacctgaatattgccattatgcttgacatggtttccaaaaaatggtactccacatatttcagtgagggtaagtattttcctgttgtcaagaatagcattgtaaaagcattttgtaataataaagaatagctttaatgatatgcttgtaactaaaataattttgtaatgtatcaaatacatttaaaacattaaaatataatctctataataagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggaagcttgaattcagctgacgtgcctcggaccgctaggaacccctagtgatggagttggccactccctctctgcgcgctcgctcgctcactgaggccgggcgaccaaaggtcgcccgacgcccgggctttgcccgggcggcctcagtgagcgagcgagcgcgcag

SEQ ID NO: 105 - DNA, 4309 bp, synthetic construct
  source 1..4309: mol_type = other DNA; note = SPARC construct
ctgcgcgctcgctcgctcactgaggccgcccgggcgtcgggcgacctttggtcgcccggcctcagtgagcgagcgagcgcgcagagagggagtggccaactccatcactaggggttcctgcggccgcacgcgtggtcaggctacctctcaggctgactgagtcatgcagcataggctgccacgtctctgggctggcggggccgtcattattcctggcctcactgcagctaaattgaagaaacgtttggtttgtgggccacgtcaaggaatgtgtaagagctgccacgttgtcgggtctgggttattgggcttttcccctccttcagagaagatttccaggcgtgtgggtggggtttcagaagaaaattgatgcctgcgtgtgagtgttccctggacctggaccagcagcggcaatattacagacccgggggttggggcagactgagccaatctctgcaccgtcaaagttatggatacagagccctggaaaaaggctgaaggataagatagctgacatttatgaagtgcttcattcatgtagcagtgggccaaatgcgtactttacacttgaggaagctgaggctggaggttgataacatgcctcaagtcttctagagttaaataactttgacccaggacccaagcccagagttctgactcaaaaactaggcctcctaaacatcctcttatatgaggttaaatttcatcttcctctgtttggccttggcctggttggtggatgctctgcttcggggacccagggccagatgacaatgggttctttgtgcccttcagacaatgggaagggctgcctggggaaagatacagtaacaaggcaacaggctgagtcagcctccaatgtgcttgaaccttcttagcttggcagccttgacattcagccagccacacaaagggtatatcaaggatgataccactagtagcagcttgtcttgtctgtacctctgaacaagaaagaggctgttctgggtcatccctccaggcctgtccagccctggcactctgtgagtcggtttaggcagcagccccggaacagatgaggcaggcagggttgggacgtttggtcaggacagcccaccaggaggaaagaaatgaaagacagagacagctttggctatgggagaaggaggaggccgggggaaggaggagacaggaggaggagggaccacggggtggaggggagatagacccagcccaaagctctgaggacccagaggccgggcgcgctccgcccgcggcgccgccccctccgtaactttcccagtctccgagggaagaggcggggtgtggggtgcggttaaaaggcgccacggcgggagacaggtctcaccggtatactctcgttgcggccccgcagcgcccgcgcgctcctctccccgactcggagcccctcggcggcgcccggcccaggacccgcctaggagcgcaggagccccagcgcagagaccccaacgccgagacccccgccccggccccgccgcgcttcctcccgacgcagtttaggacccttgttcgcgaagaggtggtgtgcggctgagacccgcgtcctcaggacggttccatcagtgcctcgatcctgccccactggaggaggaaggcagcccgaacagcgctcacctaactaacagctgctgagagctgggttccgtggccatgcacctgggactgccttgagaagcgtgagcaaaccgcccagagtagaagcgctagccaccatggattggggcacgctgcagacgatcctggggggtgtgaacaaacactccaccagcattggaaagatctggctcaccgtcctcttcatttttcgcattatgatcctcgttgtggctgcaaaggaggtgtggggagatgagcaggccgactttgtctgcaacaccctgcagccaggctgcaagaacgtgtgctacgatcactacttccccatctcccacatccggctatgggccctgcagctgatcttcgtgtccacgccagcgctcctagtggccatgcacgtggcctaccggagacatgagaagaagaggaagttcatcaagggggagataaagagtgaatttaaggacatcgaggagatcaaaacccagaaggtccgcatcgaaggctccctgtggtggacctacacaagcagcatcttcttccgggtcatcttcgaagccgccttcatgtacgtcttctatgtcatgtacgacggcttctccatgcagcggctggtgaagtgcaacgcctggccttgtcccaacactgtggactgctttgtgtcccggcccacggagaagactgtcttcacagtgttcatgattgcagtgtctggaatttgcatcctgctgaatgtcactgaattgtgttatttgctaattagatattgttctgggaagtcaaaaaagccagttggatcccgggctgactacaaagaccatgacggtgattataaagatcatgacatcgactacaaggatgacgatgacaagtaagaaatagacagcatgagagggatgaggcaacccgtgctcagctgtcaaggctcagtcgctagcatttcccaacacaaagattctgaccttaaatgcaaccatttgaaacccctgtaggcctcaggtgaaactccagatgccacaatggagctctgctcccctaaagcctcaaaacaaaggcctaattctatgcctgtcttaattttctttcacttaagttagttccactgagaccccaggctgttaggggttattggtgtaaggtactttcatattttaaacagaggatatcggcatttgtttctttctctgaggacaagagaaaaaagccaggttccacagaggacacagagaaggtttgggtgtcctcctggggttctttttgccaactttccccacgttaaaggtgaacattggttctttcatttgctttggaagttttaatctctaacagtggacaaagttaccagtgccttaaactctgttacactttttggaagtgaaaactttgtagtatgataggttattttgatgtaaagatgttctggataccattatatgttccccctgtttcagaggctcagattgtaatatgtaaatggtatgtcattcgctactatgatttaatttgaaatatggtcttttggttatgaatactttgcagcacagctgagaggctgtctgttgtattcattgtggtcatagcacctaacaacattgtagcctcaatcgagtgagacagactagaagttcctagtgatggcttatgatagcaaatggcctcatgtcaaatatttagatgtaattttgtgtaagaaatacagactggatgtaccaccaactactacctgtaatgacaggcctgtccaacacatctcccttttccatgactgtggtagccagcatcggaaagaacgctgatttaaagaggtcgcttgggaattttattgacacagtaccatttaatggggaggacaaaatggggcaggggagggagaagtttctgtcgttaaaaacagatttggaaagactggactctaaagtctgttgattaaagatgagctttgtctacttcaaaagtttgtttgcttaccccttcagcctccaattttttaagtgaaaatatagctaataacatgtgaaaagaatagaagctaaggtttagataaatattgagcagatctataggaagattgaacctgaatattgccattatgcttgacatggtttccaaaaaatggtactccacatatttcagtgagggtaagtattttcctgttgtcaagaatagcattgtaaaagcattttgtaataataaagaatagctttaatgatatgcttgtaactaaaataattttgtaatgtatcaaatacatttaaaacattaaaatataatctctataataagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggaagcttgaattcagctgacgtgcctcggaccgctaggaacccctagtgatggagttggccactccctctctgcgcgctcgctcgctcactgaggccgggcgaccaaaggtcgcccgacgcccgggctttgcccgggcggcctcagtgagcgagcgagcgcgcag

SEQ ID NO: 106 - DNA, 4453 bp, synthetic construct
  source 1..4453: mol_type = other DNA; note = TSPAN8 construct
ctgcgcgctcgctcgctcactgaggccgcccgggcgtcgggcgacctttggtcgcccggcctcagtgagcgagcgagcgcgcagagagggagtggccaactccatcactaggggttcctgcggccgcacgcgtggtccaaggactcttttttctaaacttcccttcatcttctagtttgacgcccttggtgggaaaagtgtctgagataaggaaaaggcatcctttcagttctctgatactatcttgaagcgagggatggagaaaggcaaagagagacacaggagaagcgtatcccctgggaacaggtgtctagtggagtccagtaactcacagtctctcagttccgtcagcactgtcccttgggtcgcaaatttcttccattagcccttccaccagctgtatttcaaatggggctggacaataattgtggccagtggccttgtgttgtttgtacttgcggactagtagttctcacctgtctttctctgactcctattagccactgggatttcagcagctggttcagccaattctactcaattcaacattaagttgcagtgggctagaactcatgggccgatttaacaagtgaaattctaccaagatacatcaaaaatagcaacaggactagatactcagctcattttgttttatttgtaatataccagttgtggctttagtgccagtctgattcatctctctactacaaaatgaggctctataaaggaaaatattgcaactggagtgaggaatttgaatcttataggaaggaatttgtcttctcatgaagacttcagtttaccagaagtatctattgaggaagtgtttacaagaaaatgtgccatttagctttattctaaatttgcataataactgaaccaaacaaaaaaatatagatagatagattgttctatctatagatagatagggaacattggcagtaggtggcagtaagttcccctgagcacatggaggacacagttaaatgcatttgaggtatgtgggaaatggtttaaagcagaattttatgccaacttttagtaacggaagcctaacaaatgtttgttctttcaagtgagagaagcaagcaatctggaactattcataagcttattttctgtatccttaaacatattttataatgaatgtatgatttaaatagtaagttaagtgtctgggggtactgcacacctcccttgcatacagtcaaacttcttcagggtgatggggaagaggagttataggctgccaagcaaaattgccaaactggtctcagaaattcactgcattggagagcgcgggatccttgcaacactgactttagcagttaaactagagtggttggggatgagtattctaagctctgaggacccagaggccgggcgcgctccgcccgcggcgccgccccctccgtaactttcccagtctccgagggaagaggcggggtgtggggtgcggttaaaaggcgccacggcgggagacaggtctcaccggtggcacttcgttgcggccccgcagcgcccgcgcgctcctctccccgactcggagcccctcggcggcgcccggcccaggacccgcctaggagcgcaggagccccagcgcagagaccccaacgccgagacccccgccccggccccgccgcgcttcctcccgacgcagtttaggacccttgttcgcgaagaggtggtgtgcggctgagacccgcgtcctcaggacggttccatcagtgcctcgatcctgccccactggaggaggaaggcagcccgaacagcgctcacctaactaacagctgctgagagctgggttccgtggccatgcacctgggactgccttgagaagcgtgagcaaaccgcccagagtagaagcgctagccaccatggattggggcacgctgcagacgatcctggggggtgtgaacaaacactccaccagcattggaaagatctggctcaccgtcctcttcatttttcgcattatgatcctcgttgtggctgcaaaggaggtgtggggagatgagcaggccgactttgtctgcaacaccctgcagccaggctgcaagaacgtgtgctacgatcactacttccccatctcccacatccggctatgggccctgcagctgatcttcgtgtccacgccagcgctcctagtggccatgcacgtggcctaccggagacatgagaagaagaggaagttcatcaagggggagataaagagtgaatttaaggacatcgaggagatcaaaacccagaaggtccgcatcgaaggctccctgtggtggacctacacaagcagcatcttcttccgggtcatcttcgaagccgccttcatgtacgtcttctatgtcatgtacgacggcttctccatgcagcggctggtgaagtgcaacgcctggccttgtcccaacactgtggactgctttgtgtcccggcccacggagaagactgtcttcacagtgttcatgattgcagtgtctggaatttgcatcctgctgaatgtcactgaattgtgttatttgctaattagatattgttctgggaagtcaaaaaagccagttggatcccgggctgactacaaagaccatgacggtgattataaagatcatgacatcgactacaaggatgacgatgacaagtaagaaatagacagcatgagagggatgaggcaacccgtgctcagctgtcaaggctcagtcgctagcatttcccaacacaaagattctgaccttaaatgcaaccatttgaaacccctgtaggcctcaggtgaaactccagatgccacaatggagctctgctcccctaaagcctcaaaacaaaggcctaattctatgcctgtcttaattttctttcacttaagttagttccactgagaccccaggctgttaggggttattggtgtaaggtactttcatattttaaacagaggatatcggcatttgtttctttctctgaggacaagagaaaaaagccaggttccacagaggacacagagaaggtttgggtgtcctcctggggttctttttgccaactttccccacgttaaaggtgaacattggttctttcatttgctttggaagttttaatctctaacagtggacaaagttaccagtgccttaaactctgttacactttttggaagtgaaaactttgtagtatgataggttattttgatgtaaagatgttctggataccattatatgttccccctgtttcagaggctcagattgtaatatgtaaatggtatgtcattcgctactatgatttaatttgaaatatggtcttttggttatgaatactttgcagcacagctgagaggctgtctgttgtattcattgtggtcatagcacctaacaacattgtagcctcaatcgagtgagacagactagaagttcctagtgatggcttatgatagcaaatggcctcatgtcaaatatttagatgtaattttgtgtaagaaatacagactggatgtaccaccaactactacctgtaatgacaggcctgtccaacacatctcccttttccatgactgtggtagccagcatcggaaagaacgctgatttaaagaggtcgcttgggaattttattgacacagtaccatttaatggggaggacaaaatggggcaggggagggagaagtttctgtcgttaaaaacagatttggaaagactggactctaaagtctgttgattaaagatgagctttgtctacttcaaaagtttgtttgcttaccccttcagcctccaattttttaagtgaaaatatagctaataacatgtgaaaagaatagaagctaaggtttagataaatattgagcagatctataggaagattgaacctgaatattgccattatgcttgacatggtttccaaaaaatggtactccacatatttcagtgagggtaagtattttcctgttgtcaagaatagcattgtaaaagcattttgtaataataaagaatagctttaatgatatgcttgtaactaaaataattttgtaatgtatcaaatacatttaaaacattaaaatataatctctataataagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggaagcttgaattcagctgacgtgcctcggaccgctaggaacccctagtgatggagttggccactccctctctgcgcgctcgctcgctcactgaggccgggcgaccaaaggtcgcccgacgcccgggctttgcccgggcggcctcagtgagcgagcgagcgcgcag

SEQ ID NO: 107 - DNA, 4317 bp, synthetic construct
  source 1..4317: mol_type = other DNA; note = VIM promoter
ctgcgcgctcgctcgctcactgaggccgcccgggcgtcgggcgacctttggtcgcccggcctcagtgagcgagcgagcgcgcagagagggagtggccaactccatcactaggggttcctgcggccgcacgcgtggtattcacaatgcattccctctgcccaccacattaattatcaactccttttcctggcatttactcatccaacgcatggccccacgttaactttcagttccctttctcccctacaaatactccataatccagcaaccctgggatccctgagatgatgaagaggaccagtgcccattccaggagacatcaccgcagccctgaggaatcggctatgggcaccagcagggcacagtgccacacctcgccaatgccttgtcctccttttccatagtgagtcagtcagcaagcgtgtagaagtgagttccacactctcttcctcccatagggagatcacttttctcattctaagggttccaggcacactcacaatggtggcatttgctgagcagtggcttgaataaagggctctcagaaagcaagatgtaactcagagcataggctagccccaggaatgctcttggggaatgacctgcagcctcccagtgaaagagagaataaaagaaagccccagcaggcgagctgggcagtagagagtcctgtaattccaccttggcaagcaccatttgcaagaacgaactgggataaggtaaacaaaatattgcctaaaagaggcttgtccaaagaagtcagaatacgctcttcatttacctctaaatttcagtacaccataaatctaaatactcaaaaaaacctgtgccttttcaatcaaggtcaattgcacgaattcttttggaaaacaggacctatggcatttcccagacaaatcaccgtgaaccctgtactgtgcattgctgtcctaaaatccaaagattctgtcatttgtgttacataattgcctttcatttgaactcattaatcaaattggggtttttaagcaacacctaattaattctttaactggctcatccactgatcactgagttctattttgaaactacggacgtcgagtttcctctttcacccagaattttcagatcttgtttaaaaagttgggtgtggtttcatggggggagggggaagagcgagaggagaccagagggacgggggcggggactctgcaagaaaaaccttcccggtgcaatcgtgatctaagctctgaggacccagaggccgggcgcgctccgcccgcggcgccgccccctccgtaactttcccagtctccgagggaagaggcggggtgtggggtgcggttaaaaggcgccacggcgggagacaggtctcaccggttttcaggtgttgcggccccgcagcgcccgcgcgctcctctccccgactcggagcccctcggcggcgcccggcccaggacccgcctaggagcgcaggagccccagcgcagagaccccaacgccgagacccccgccccggccccgccgcgcttcctcccgacgcagtttaggacccttgttcgcgaagaggtggtgtgcggctgagacccgcgtcctcaggacggttccatcagtgcctcgatcctgccccactggaggaggaaggcagcccgaacagcgctcacctaactaacagctgctgagagctgggttccgtggccatgcacctgggactgccttgagaagcgtgagcaaaccgcccagagtagaagcgctagccaccatggattggggcacgctgcagacgatcctggggggtgtgaacaaacactccaccagcattggaaagatctggctcaccgtcctcttcatttttcgcattatgatcctcgttgtggctgcaaaggaggtgtggggagatgagcaggccgactttgtctgcaacaccctgcagccaggctgcaagaacgtgtgctacgatcactacttccccatctcccacatccggctatgggccctgcagctgatcttcgtgtccacgccagcgctcctagtggccatgcacgtggcctaccggagacatgagaagaagaggaagttcatcaagggggagataaagagtgaatttaaggacatcgaggagatcaaaacccagaaggtccgcatcgaaggctccctgtggtggacctacacaagcagcatcttcttccgggtcatcttcgaagccgccttcatgtacgtcttctatgtcatgtacgacggcttctccatgcagcggctggtgaagtgcaacgcctggccttgtcccaacactgtggactgctttgtgtcccggcccacggagaagactgtcttcacagtgttcatgattgcagtgtctggaatttgcatcctgctgaatgtcactgaattgtgttatttgctaattagatattgttctgggaagtcaaaaaagccagttggatcccgggctgactacaaagaccatgacggtgattataaagatcatgacatcgactacaaggatgacgatgacaagtaagaaatagacagcatgagagggatgaggcaacccgtgctcagctgtcaaggctcagtcgctagcatttcccaacacaaagattctgaccttaaatgcaaccatttgaaacccctgtaggcctcaggtgaaactccagatgccacaatggagctctgctcccctaaagcctcaaaacaaaggcctaattctatgcctgtcttaattttctttcacttaagttagttccactgagaccccaggctgttaggggttattggtgtaaggtactttcatattttaaacagaggatatcggcatttgtttctttctctgaggacaagagaaaaaagccaggttccacagaggacacagagaaggtttgggtgtcctcctggggttctttttgccaactttccccacgttaaaggtgaacattggttctttcatttgctttggaagttttaatctctaacagtggacaaagttaccagtgccttaaactctgttacactttttggaagtgaaaactttgtagtatgataggttattttgatgtaaagatgttctggataccattatatgttccccctgtttcagaggctcagattgtaatatgtaaatggtatgtcattcgctactatgatttaatttgaaatatggtcttttggttatgaatactttgcagcacagctgagaggctgtctgttgtattcattgtggtcatagcacctaacaacattgtagcctcaatcgagtgagacagactagaagttcctagtgatggcttatgatagcaaatggcctcatgtcaaatatttagatgtaattttgtgtaagaaatacagactggatgtaccaccaactactacctgtaatgacaggcctgtccaacacatctcccttttccatgactgtggtagccagcatcggaaagaacgctgatttaaagaggtcgcttgggaattttattgacacagtaccatttaatggggaggacaaaatggggcaggggagggagaagtttctgtcgttaaaaacagatttggaaagactggactctaaagtctgttgattaaagatgagctttgtctacttcaaaagtttgtttgcttaccccttcagcctccaattttttaagtgaaaatatagctaataacatgtgaaaagaatagaagctaaggtttagataaatattgagcagatctataggaagattgaacctgaatattgccattatgcttgacatggtttccaaaaaatggtactccacatatttcagtgagggtaagtattttcctgttgtcaagaatagcattgtaaaagcattttgtaataataaagaatagctttaatgatatgcttgtaactaaaataattttgtaatgtatcaaatacatttaaaacattaaaatataatctctataataagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggaagcttgaattcagctgacgtgcctcggaccgctaggaacccctagtgatggagttggccactccctctctgcgcgctcgctcgctcactgaggccgggcgaccaaaggtcgcccgacgcccgggctttgcccgggcggcctcagtgagcgagcgagcgcgcag

SEQ ID NO: 108-115 - skipped (8 entries)

SEQ ID NO: 116 - DNA, 145 bp, synthetic construct
  source 1..145: mol_type = other DNA; note = Synthetic sequence
aggaacccctagtgatggagttggccactccctctctgcgcgctcgctcgctcactgaggccgcccgggcaaagcccgggcgtcgggcgacctttggtcgcccggcctcagtgagcgagcgagcgcgcagagagggagtggccaa

SEQ ID NO: 117 - DNA, 1044 bp, synthetic construct; identical to SEQ ID NO: 65
  source 1..1044: mol_type = other DNA; note = Exemplary Regulatory sequence C3

SEQ ID NO: 118 - DNA, 1044 bp, synthetic construct; identical to SEQ ID NO: 65
  source 1..1044: mol_type = other DNA; note = Exemplary Regulatory sequence D7